(19) United States
(12) Patent Application Publication
(10) Pub. No.: US 2025/0163006 A1
(43) Pub. Date: May 22, 2025

(54) New Coolants, and Preparations Containing Same

(71) Applicant: Symrise AG, Holzminden (DE)

(72) Inventors: Judith MINAKAR, Höxter (DE); Benoit JOIN, Holzminden (DE); Joachim HANS, Holzminden (DE); Michael BACKES, Holzminden (DE); Rebecca WIEBUSCH, Holzminden (DE); Dominik STUHLMANN, Holzminden (DE); Gabriela MATUSZKO, Braunschweig (DE)

(21) Appl. No.: 18/833,384

(22) PCT Filed: Jan. 27, 2023
(86) PCT No.: PCT/EP2023/052047

§ 371 Date: Jul. 25, 2024

(30) Foreign Application Priority Data
Jan. 28, 2022 (WO) PCT/EP2022/052139

(51) Int. Cl. C07D 253/07; A23G 3/36; A23G 4/06; A23L 9/10; A23L 27/20; A61K 8/49; A61K 31/421; A61K 31/53; A61Q 5/00; A61Q 5/02; A61Q 5/06; A61Q 5/12; A61Q 9/02; A61Q 11/00; A61Q 15/00; A61Q 17/00; A61Q 17/04; A61Q 19/00; A61Q 19/08; A61Q 19/10; C07D 233/84; C07D 241/18; C07D 253/10; C07D 263/46; C07D 403/12; C07D 405/12; C07D 409/04; C07D 409/12; C07D 409/14; C07D 413/04

(52) U.S. Cl. CPC C07D 253/07; A23G 3/36; A23G 4/06; A23L 9/12; A23L 27/2054; A23L 27/2056; A61K 8/49; A61K 8/4966; A61K 31/421; A61K 31/53; A61Q 5/006; A61Q 5/02; A61Q 5/06; A61Q 5/12; A61Q 9/02; A61Q 11/00; A61Q 15/00; A61Q 17/005; A61Q 17/04; A61Q 19/002; A61Q 19/004; A61Q 19/007; A61Q 19/08; A61Q 19/10; C07D 233/84; C07D 241/18; C07D 253/10; C07D 263/46; C07D 403/12; C07D 405/12; C07D 409/04; C07D 409/12; C07D 409/14; C07D 413/04

(57) ABSTRACT

The present invention relates to novel physiological coolants, mixtures of coolants containing these novel agents, mixtures of the coolants with flavors, the use of these coolants, and articles and end preparations for the consumer containing the physiological coolants or mixtures of coolants.

### FIELD OF THE INVENTION

The present invention is in the field of physiological coolants and relates to new representatives of this group, the use of these coolants and articles and preparations comprising these coolants.

### TECHNOLOGICAL BACKGROUND

Physiological coolants are regularly used to create a cool sensory impression on the skin or mucous membrane, for example on the mucous membrane in the mouth, nose and/or throat, although no physical cooling actually takes place, such as with the evaporation of solvents. Both individual components and mixtures can be used as physiological coolants. It should be noted that not all compounds that influence receptors in vitro, which are (also) involved in mediating a physiological cooling effect, actually produce such an effect in vivo on the skin or mucous membranes. In particular, such an effect will not always be identical. This means, for example, that the strength of the mediated physiological cooling effect and the progression of the strength of the cooling effect over time cannot be inferred solely from the fact that a particular compound is an agonist of a receptor involved in mediating a cooling effect.

TRP channels play an important role in the perception of temperature (hot-cold). TRP channels (transient receptor potential channels) are an extensive family of cellular ion channels that can be divided into seven subfamilies.

The cold menthol receptor TRPM8 (also known as the cold membrane receptor (CMR1)) belongs to the family of “transient receptor potential ion channels”, is specifically expressed in a special group of neurons and forms pores in the cell membrane (four units are clustered together to form a tetramer), which selectively allow Ca²⁺ ions to pass through. The protein has six transmembrane domains and a cytoplasmic C- and N-terminus. This receptor is stimulated by low temperatures (preferably 10 to 25° C.), resulting in signal transduction, which is interpreted by the nervous system as a cold sensation.

There is evidence that several TRP channels are important for growth control. Changes in the expression of some of these channels can contribute to the development of cancer. For example, the expression of the TRPM8 gene is upregulated in prostate carcinomas. Accordingly, TRPM8 are also attractive targets for the treatment of prostate or bladder cancer.

### STATE OF THE ART

Cooling compounds, such as menthol, have long played an important role in the flavor and fragrance industry to create an association with freshness and cleanliness.

The best known physiologically effective coolant is L-menthol. The compound menthol has been shown to act as a natural modulator of the TRPM8 receptor. The application of menthol activates TRPM8, causing a Ca²⁺ influx into the cold-sensitive neurons. The resulting electrical signal is then perceived as a cold sensation.

However, menthol has a number of disadvantages, such as a strong odor, high volatility and, in higher concentrations, a bitter and/or pungent taste of its own and a skin-irritating effect. Excessive concentrations of menthol can also cause irritation and an anaesthetic effect on the skin or mucous membranes.

Strong coolants that do not have the disadvantageous properties of L-menthol have been sought before.

For example, lactic acid esters of menthol(s) according to DE 2608226 A1 and mixed carbonates with menthol(s) and polyols according to DE 4226043 A1 and menthone ketals according to EP 0507190 B1 have been described.

In addition, menthol derivatives with a similar effect have been described in various publications.

Although menthyl monoesters of diacids according to U.S. Pat. Nos. 5,725,865 and 5,843,466 are interesting naturally occurring alternatives, they cannot achieve the strength of the previously described coolants in sensory tests.

It was found that the compounds L-menthanecarboxylic acid N-ethylamide (“WS-3”) and in particular Na-(L-menthanecarbonyl)glycine ethyl ester (“WS-5”) are strong coolants. However, the latter has the disadvantage of being sensitive to hydrolysis and forming the corresponding free acid Na-(L-menthanecarbonyl)glycine, which itself only has a very weak cooling effect. Despite the detailed investigations described above, a systematic prediction of the properties of potential coolants, in particular their bitterness and/or their other trigeminal effects, is not possible and has not been described. For example, many molecules belonging to the class of menthanecarboxylic acid amides have a strong cooling effect, but often show pronounced bitter notes at the same time, e.g. menthanecarboxylic acid N-(alkyloxyalkyl)amides according to JP 2004 059474 A2 or are also highly irritating, such as the N-[[5-methyl-2-(1-methylethyl)cyclohexyl]carbonyl]glycine ethyl ester according to US 2005 0222256 A1, so that such compounds are not suitable for use in food preparations or the like.

Na-(menthancarbonyl)alkyloxyalkylamides were described in JP 2004 059474 A2. However, with a strong cooling effect and high hydrolysis stability, these have the disadvantage of being very bitter and therefore cannot be used in foodstuffs or cosmetic products used for facial care.

Furthermore, menthylglyoxylates and their hydrates have been described as cooling substances in JP 2005 343795 A2.

Overviews of the coolants produced and used to date are known to the skilled person.

There are also isolated compounds structurally unrelated to menthol that cause significant TRPM8 modulation, such as the coolant WS-23 or the compounds listed in patent application WO 2007 019719 A1.

However, many of the modulators of TRPM8 found to date have shortcomings in terms of potency, duration of action, skin/mucous membrane irritation, odor, taste, solubility, and volatility.

WO 2010 026094 A1 discloses individual compounds for modulating the TRPM8 receptor.

Further compounds for modulating the TRPM8 receptor are also proposed in WO 2011 061330 A2.

Special coolants with the carboxamide structure (I)

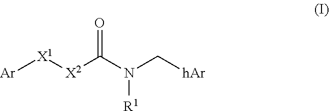

are also known from WO 2012 061698 A1.

On the oral mucosa, many to all of the conventional cooling substances mentioned above and known from the state of the art exhibit more or less identical cooling behavior. The cooling sensation of freshness imparted by them sets in after about 0.5 minutes, but then levels off again relatively quickly after a peak of 3 to 5 minutes, whereby the cooling is clearly perceptible for a maximum of 30 minutes and experience has shown that the intensity and duration can be influenced only slightly by changing the dosage. On the consumer side, however, there is a desire for a particularly long-lasting cooling effect, which is associated with a corresponding feeling of freshness and well-being for the user.

### Task of the Invention

The primary task of the present invention was therefore to identify new substances which have a particular physiological cooling effect, preferably those which lead to a modulation of the TRPM8 receptor (so-called modulators), which can be used as alternatives, preferably as more suitable agents, to the previously known modulators. Such compounds should also be particularly suitable for applications in the fields of cosmetics, nutrition, textiles, OTC products (e.g., burn ointments), pharmaceuticals (e.g., in the field of tumor treatment, bladder weakness) or packaging. The compounds or mixtures of compounds to be disclosed should preferably have as weak an inherent taste as possible, in particular have little or no bitter taste and be as non-irritating as possible.

To solve the problem according to the invention, the search was primarily for active ingredients that can impart a particularly long-lasting cooling sensation. Preferably, these active ingredients should also be able to impart particularly intense and/or rapid cooling sensations. The coolants should be efficient, i.e., they should have a high cooling effect or sensation even at low concentrations.

Another task was to compensate for the off-flavors that many flavors, especially sweeteners such as representatives of the stevioside group, have. This applies in particular to their bitter, astringent and metallic aftertaste.

The problem is solved by the objects of the independent patent claims. Further aspects of the present invention are apparent from the wording of the dependent patent claims, the following description, and the examples.

### DETAILED DESCRIPTION OF THE INVENTION

The primary task of the present invention is solved according to the invention by a physiological coolant selected from the group consisting of compounds represented by the general formulae (I) to (IV)

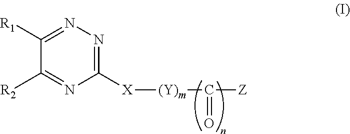

or the general formula (II)

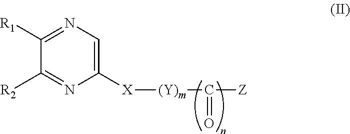

or the general formula (III)

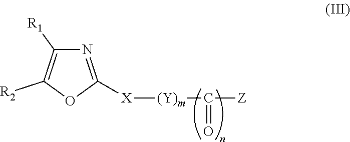

or the general formula (IV)

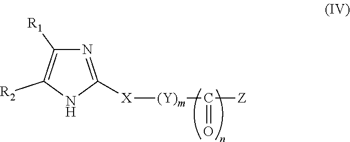

where in each of the formulae (I) to (IV)
the radicals R1 and R2 may be identical or different and have the following meanings independently of one another:

### R1 H; or

a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group —R(C═O)—; or
an optionally substituted cycloalkyl group; or
an optionally substituted aryl group; or
an optionally substituted heterocycloalkyl group; or
is an optionally substituted heteroaryl group;

### R2 H; or

a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is an optionally substituted heteroaryl group; or
or R1 and R2 together with the C atoms to which they are bonded form a conjugated or non-conjugated ring system;

### X S; or

SO; or
SO₂; or
NH; or
O; or
an optionally substituted linear or branched alkyl group
an optionally substituted linear or branched alkenyl group
an optionally substituted linear or branched alkylthio group; or
an optionally substituted cycloalkyl group; or
an optionally substituted heterocycloalkyl group; or
an optionally substituted aryl group; or
is an optionally substituted heteroaryl group;
   Y is an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—; or
an optionally substituted alkylaryl group; or
is an optionally substituted alkylheteroaryl group;

### Z NH₂; or

an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
   m is 0 or 1; and/or
   n is 0or 1;
with the proviso that
if m=0, Y is omitted; or
if m=1, Y occurs once in each case; or
if n=0, —(C═O)— is omitted; or
if n=1, —(C═O)— occurs once; or
if m=0 and n=0, Y is omitted and —(C═O)— is omitted; or
if m=1 and n=1, Y occurs once and —(C═O)— occurs once; or
if m=0 and n=1, Y is omitted and —(C═O) occurs once; or
if m=1 and n=0, Y occurs once and —(C═O)— is omitted;
whereby
the group Q is a radical selected from the group consisting of: halogen, —OA, —SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
wherein
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁- to C₆-alkoxy group, in particular optionally substituted C₁-, C₂-, C₃-or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one,
two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group; the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group, is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
   Ra and/or Rb an optionally substituted linear or branched alkyl group, preferably a C₁ to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
   wherein the substituents are selected independently of one another;
   and their salts, preferably acid addition salts, with inorganic or organic acids.

The coolants according to the invention, represented by the general formulae (I) to (IV), can be present both in stereoisomerically pure form or as mixtures of different stereoisomers.

In a preferred variant according to the first aspect of the present invention, it is a physiological coolant according to one of the general formulae (I) and (II), wherein X is selected from the group consisting of S, SO, NH and O. Even more preferred is a coolant according to the present invention, wherein in the general formulae (I) and (II) X is S.

In an alternative preferred variant according to the first aspect of the present invention, it is a physiological coolant according to one of the general formulae (III) and (IV), wherein X is selected from the group consisting of S, SO₂, an optionally substituted linear or branched alkyl group, an optionally substituted cycloalkyl group and piperidinyl.

In an even more preferred variant according to the first aspect of the present invention, it is a physiological coolant selected from the group consisting of compounds represented by the general formula (V)

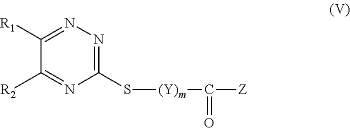

or by the general formula (VI)

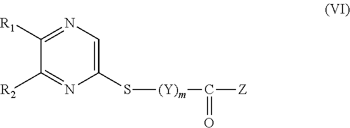

where in formulae (V) and (VI) in each case
the radicals R1 and R2 may be identical or different and have the following meanings independently of one another:
   R1 as defined for formulae (I) to (IV);
   R2 as defined for formulae (I) to (IV);
   Y is an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—; or
an optionally substituted alkylaryl group; or
is an optionally substituted alkylheteroaryl group;

### Z NH₂; or

an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
   m is 0 or 1;
with the proviso that
if m=0, Y is omitted; or
if m=1, Y occurs once in each case;
   whereby
the group Q is a radical selected from the group consisting of: halogen, —OA, —SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁- to C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group, is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
   Ra and/or Rb an optionally substituted linear or branched alkyl group, preferably a C₁ to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
   where the substituents are selected independently of each other;
   and their salts, preferably acid addition salts, with inorganic or organic acids;
   or
   given by the general formula (VII)

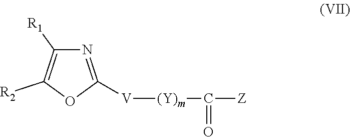

or by the general formula (VIII)

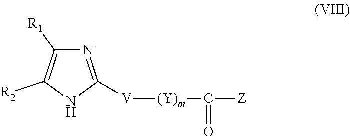

where in each of the formulae (VII) and (VIII)
the radicals R1 and R2 may be identical or different and have the following meanings independently of one another:
   R1 as defined for formulae (I) to (IV);
   R2 as defined for formulae (I) to (IV);

### V S; or

SO₂; or
an optionally substituted linear or branched alkyl group; or
an optionally substituted cycloalkyl group; or
is piperidinyl;
   Y is an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—;
an optionally substituted alkylaryl group; or
is an optionally substituted alkylheteroaryl group;

### Z NH₂; or

an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
   m is 0 or 1;
with the proviso that
if m=0, Y is omitted; or
if m=1, Y occurs once in each case;
   wherein
the group Q is a radical selected from the group consisting of: halogen, —OA, —SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
wherein
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁- to C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁-to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group,
is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group, is in particular an optionally substituted C₁-, C₂-, C₃- or C₄-alkylthio group;
in the acyl group R—(C═O)— the radical R represents hydrogen or an optionally substituted linear or branched C₁- to C₁o-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
   Ra and/or Rb an optionally substituted linear or branched alkyl group, preferably a C₁ to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group;
Ra and Rb, as defined above, are linked together and form a saturated or unsaturated ring;
   where the substituents are selected independently of one another;
   and their salts, preferably acid addition salts, with inorganic or organic acids.

The coolants according to the general formulae (V) to (VIII) of the invention can be present both in stereoisomerically pure form or as mixtures of different stereoisomers.

In an even more preferred variant according to the first aspect of the present invention, it is a physiological coolant selected from the group consisting of compounds represented by the general formula (Va)

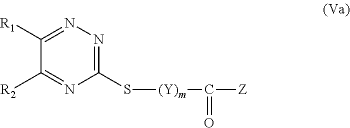

or by the general formula (VIa)

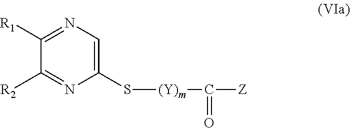

where in formulae (Va) and (VIa) in each case
the radicals R1 and R2 may be identical or different and have the following meanings independently of one another:
   R1 as defined for formulae (I) to (IV);
   R2 as defined for formulae (I) to (IV);
or R1 and R2 together with the C atoms to which they are bonded form a conjugated or non-conjugated ring system;
   Y is an optionally substituted branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—; or
an optionally substituted alkylaryl group; or
is an optionally substituted alkylheteroaryl group;
   Z in the general formula (Va) NH₂; or
an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
   Z in the general formula (VIa) NH₂; or
an NHRa group or an NRaRb group, with the exception of —NH-phenyl and —N(CH3)-phenyl; or
an optionally substituted linear or branched alkyl group; or
an optionally substituted linear or branched alkenyl group; or
an optionally substituted linear or branched alkylthio group; or
an optionally substituted linear or branched alkoxy group, with the exception of —OC2H5 and —C(CH3)3; or
an optionally substituted cycloalkyl group; or
an optionally substituted heterocycloalkyl group; or
an optionally substituted aryl group; or
is an optionally substituted heteroaryl group; or NH and together with the X forms a heterocyclic ring;
   m 1 is;
   whereby
the group Q is a radical selected from the group consisting of: halogen, —OA, -SA, —NBB, —CF₃, —CN, —OCN, —SCN, —NO, —NO₂, —S(O)2a, —S(O)₂OA, —OS(O)2a, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group, optionally substituted linear or branched alkoxy group, in particular optionally substituted C₁- to C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁-alkylthio group, in particular an optionally substituted C₁- to C₆-alkylthio group, is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
   Ra and/or Rb an optionally substituted linear or branched alkyl group, preferably a C1 to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
   wherein the substituents are selected independently of one another;
   and their salts, in particular acid addition salts, with inorganic or organic acids, whereby the coolants may be present in stereoisomerically pure form or as mixtures of different stereoisomers;
   or represented by the general formula (VIIa)

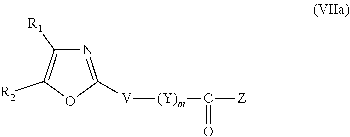

or by the general formula (VIIIa)

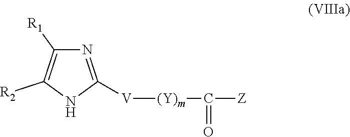

where in each of the formulae (VIIa) and (VIIIa)
the radicals R1 and R2 may be identical or different and have the following meanings independently of one another:
R1 as defined for formulas (I) to (IV);
R2 as defined for formulas (I) to (IV);
or R1 and R2 together with the C atoms to which they are bonded form a conjugated or non-conjugated ring system;

### V S; or

SO₂; or
an optionally substituted linear or branched alkyl group; or
an optionally substituted cycloalkyl group; or
is piperidinyl;
   Y is an optionally substituted branched alkyl group; or
an optionally substituted linear or branched alkenyl group; or
an optionally substituted linear or branched alkynyl group; or
an optionally substituted linear or branched alkoxy group; or
is an optionally substituted acyl group R—(C═O)—;

### Z NH₂; or

an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
   m 1 is;
   wherein
the group Q is a radical selected from the group consisting of: halogen, —OA, -SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group, optionally substituted linear or branched alkoxy group, in particular optionally substituted C₁- to C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to Co-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group, is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
   Ra and/or Rb an optionally substituted linear or branched alkyl group; preferably a C1 to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
   wherein the substituents are selected independently of one another;
   and their salts, in particular acid addition salts, with inorganic or organic acids, whereby the coolants may be present in stereoisomerically pure form or as mixtures of different stereoisomers.

The coolants according to the general formulae (Va) to (VIIIa) of the invention can be present both in stereoisomerically pure form or as mixtures of different stereoisomers.

The present invention also includes physiological coolants in which the oxygen atom in the oxazole ring of the basic structure of the general formulae (III), (VII) or (VIIa) is replaced by a sulphur atom, i.e., the oxazole ring of the basic structure of the general formulae (III), (VII) or (VIIa) is a thiazole ring.

Of the compounds of the general formulae (I) to (VIII) and (Va) to (VIIIa), those compounds in which the heterocyclic ring of the basic structure of the general formulae (I) to (VIII) and (Va) to (VIIIa) has at least two nitrogen atoms, i.e. compounds of the general formulae (I), (V) and (Va) with a triazine ring in the basic structure or compounds of the general formulae (II), (VI) and (VIa) with a pyrazine ring in the basic structure, are particularly preferred due to the free electron pairs which are suitable for forming a covalent bond (so-called Lewis bases). i.e. compounds of the general formulae (I), (V) and (Va) with a triazine ring in the basic structure or compounds of the general formulae (II), (VI) and (VIa) with a pyrazine ring (diazine) in the basic structure or compounds of the general formulae (IV), (VIII) and (Villa) with an imidazole ring. Such compounds exhibit particularly pronounced cooling attributes as described below.

Due to their Lewis base character, compounds of the general formulae (I), (V) and (Va) with a triazine ring in the basic structure and compounds of the general formulae (III), (VII) and (VIIa) with an oxazole ring in the basic structure are most preferred. Such compounds are characterized by a particularly intensive cooling effect.

In the context of the present invention, in particular for the definition of the general formulae (I) to (VIII) and (Va) to (Villa), the following general meanings apply:

The term “or” or “and/or” is used as a function word to indicate that two words or expressions should be taken together or separately.

The terms “comprising”, “with”, “including” and “containing” are to be understood as open terms, i.e., “comprising”, “including” or “containing”, but not “limited to”.

The end points of all areas that are directed to the same component or property can be combined inclusively and independently of each other.

The term “compound(s)” or “compound(s) of the present invention” refers to all compounds encompassed by the structural formula Formula (I) and/or Formula (II) disclosed herein and includes any subgenus and any specific compounds within the formula whose structure is disclosed herein. The compounds can be identified by either their chemical structure and/or their chemical name. When the chemical structure and chemical name are in conflict, the chemical structure determines the identity of the compound. The compounds described herein may include one or more chiral centers and/or double bonds and therefore may exist as stereoisomers, such as double bond isomers, i.e., geometric isomers, enantiomers, or diastereomers. Accordingly, the chemical structures of general formula (I) and/or formula (II) shown herein comprise all possible enantiomers and diastereomers or stereoisomers.

The term “at least one coolant” in the context of the present invention means, for example, that a composition contains at least one coolant, but may also contain two, three, four or even several different coolants.

The term “alkyl” alone or as part of another substituent according to the present invention refers to a saturated or mono- or polyunsaturated linear or branched monovalent hydrocarbon radical obtained by removing a hydrogen atom from a single carbon atom of a corresponding starting alkane.

In a preferred variant, the term “alkyl” also includes all alkyl moieties in radicals derived therefrom, such as alkoxy, alkylthio, alkylsulphonyl saturated linear or branched hydrocarbon radicals having 1 to 10, 1 to 8, 1 to 6 or 1 to 4 carbon atoms.

If the alkyl residue is further bonded to another atom, it becomes an alkylene residue or an alkyl group. In other words, the term “alkylene” also refers to a divalent alkyl. For example, —CH₂CH₃ is an ethyl, while —CH₂CH₂— is an ethylene.

The term “alkylene” alone or as part of another substituent refers to a saturated linear or branched divalent hydrocarbon radical obtained by removing two hydrogen atoms from a single carbon atom or two different carbon atoms of a starting alkane.

In preferred variants according to the present invention, an alkyl group or an alkylene group comprises 1 to 10 carbon atoms. In other still more preferred variants, an alkyl group or alkylene group comprises from 1 to 6 carbon atoms. Most preferred are alkyl groups or alkylene groups with 1 to 4 carbon atoms.

Preferred alkyl radicals or alkyl groups include, but are not limited to: C₁- to C₆-alkyl comprising methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
C₁- to C₆-alkoxy comprising C₁- to C₄-alkoxy, such as methoxy, ethoxy, n-propoxy, 1-methylethoxy, butoxy, 1-methylpropoxy, 2-methylpropoxy or 1,1-dimethylethoxy; as well as pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

Most preferred according to the invention are saturated linear or branched C₁- to C₆-alkyl groups or saturated linear or branched C₁- to C₆-alkylene groups.

The term “alkyl” or “alkylene” further includes radicals or groups with any degree of saturation, i.e., groups with only single carbon-carbon bonds (“alkyl” or “alkylene”), groups with one or more double carbon-carbon bonds (“alkenyl”), radicals with one or more triple carbon-carbon bonds (“alkynyl”) and groups with a mixture of single, double and/or triple carbon-carbon bonds.

The term “alkenyl” alone or as part of another substituent according to the present invention refers to an unsaturated linear or branched monovalent hydrocarbon radical having at least one carbon-carbon double bond (C═C double bond). The remainder can be in either the cis or trans conformation around the double bond(s). So that the term “alkenyl” also includes the corresponding cis/trans isomers.

Typical alkenyl radicals or Alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl and the like.

In preferred variants according to the present invention, an alkenyl group comprises 2 to 10 carbon atoms. In other preferred variants, an alkenyl group comprises 2 to 6 carbon atoms. In still further preferred variants, an alkenyl group comprises 2 to 4 carbon atoms.

Most preferred according to the invention are mono- or di-unsaturated linear or branched C₁- to C₆-alkenyl groups.

The term “alkynyl” alone or as part of another substituent according to the present invention refers to an unsaturated linear or branched monovalent hydrocarbon radical having at least one carbon-carbon triple bond (C═C triple bond).

Typical alkynyl residues or alkynyl groups include, but are not limited to, ethynyl; propynyls such as prop-1-yn-1-yl, prop-2-in-1-yl, etc.; butynyls such as but-1-in-1-yl, but-1-in-3-yl, but-3-in-1-yl, and the like.

In preferred variants according to the present invention, an alkynyl group comprises 2 to 10 carbon atoms. In other preferred variants, an alkynyl group comprises from 2 to 6 carbon atoms. In still further preferred variants, an alkynyl group comprises 2 to 4 carbon atoms.

The term “alkoxy” alone or as part of another substituent according to the present invention refers to a radical of the formula —O—R, wherein R is alkyl or substituted alkyl as defined herein.

The term “alkylthio” or “thioalkoxy” alone or as part of another substituent according to the present invention refers to a radical of the formula —S—R, wherein R is alkyl or substituted alkyl as defined herein.

According to the invention, the term “alkyl” or “alkylene” also includes heteroalkyl radicals or heteroalkyl groups. The term “heteroalkyl” by itself or as part of other substituents refers to alkyl groups in which one or more of the carbon atom(s) is/are independently replaced by the same or a different heteroatom or by the same or a different heteroatomic group(s). Typical heteroatoms or heteroatomic groups that may replace the carbon atoms include, but are not limited to, —O—, —S—, —N—, —Si—, —NH—, —S(O)—, —S(O)₂—, —S(O)NH—, —S(O)₂NH— and the like, and combinations thereof. The heteroatoms or heteroatomic groups may be located at any internal position of the alkyl group. Typical heteroatomic groups that may be included in these groups include, but are not limited to, —O—, —S—, —O—O—, —S—S—, —O—S—, —NRR—, ═NN═, —N═N—, —N═N—NRR, —PR—, —P(O)₂—, —POR—, —O—P(O)₂—, —SO—, —SO₂—, —SR₂OR—and the like, wherein R is independently hydrogen, alkyl, substituted alkyl, aryl, substituted aryl, substituted aryl, substituted aryl and the like, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl as defined herein.

The alkyl group or the alkylene group, as defined above, may also be substituted.

The term “acyl” alone or as part of another substituent according to the present invention refers to a radical —R(C═O)—, wherein R is hydrogen, alkyl, substituted alkyl, cycloalkyl, substituted cycloalkyl, aryl, substituted aryl, arylalkyl, substituted arylalkyl, heteroalkyl, substituted heteroalkyl, heteroarylalkyl or substituted heteroarylalkyl, as defined herein.

Representative examples include, but are not limited to, formyl, acetyl, propionyl, butyryl, valeryl, benzoyl, cyclohexylcarbonyl, cyclohexylmethylcarbonyl, benzylcarbonyl, and the like.

The term “cycloalkyl” alone or as part of another substituent according to the present invention refers to a saturated or mono- or polyunsaturated, non-aromatic, cyclic monovalent hydrocarbon radical in which the carbon atoms are linked together in a ring and which has no heteroatom.

The carbon ring can occur as a monocyclic compound, which has only a single ring, or as a polycyclic compound, which has two or more rings.

In a preferred variant, the term “cycloalkyl” comprises a three- to ten-membered monocyclic cycloalkyl radical or cycloalkyl group or a nine- to twelve-membered polycyclic cycloalkyl radical or cycloalkyl group. In other still more preferred variants, the cycloalkyl moiety comprises a three-, four-, five-, six- or seven-membered monocyclic cycloalkyl moiety or a nine- to twelve-membered bicyclic cycloalkyl moiety.

In a preferred variant according to the present invention, a cycloalkyl radical or cycloalkyl group comprises 3 to 20 carbon atoms. In an even more preferred variant, a cycloalkyl radical comprises 3 to 15 carbon atoms. In a most preferred variant, a cycloalkyl radical comprises 3 to 10 carbon atoms. Most preferred are monocyclic C₃- to C₇-cycloalkyl groups.

Typical cycloalkyl groups include, but are not limited to, saturated carbocyclic radicals having 3 to 20 carbon atoms, such as C₃- to C₁₂-carbocyclyl, comprising cyclopropyl, cyclobutyl, cyclopentyl, cyclohexyl, cycloheptyl, cyclooctyl, cyclononyl, cyclodecyl, cycloundecyl and cyclododecyl; preferred are cyclopentyl, cyclohexyl, cycloheptyl, and cyclopropyl-methyl, cyclopropyl-ethyl, cyclobutyl-methyl, cyclobutyl-ethyl, cyclopentyl-methyl, cyclopentyl-ethyl, cyclohexyl-methyl, or C₃- to C₇-carbocyclyl, comprising cyclopropyl, cyclobutyl, cyclopentyl, cyclohexyl, cycloheptyl, cyclopropyl-methyl, cyclopropyl-ethyl, cyclobutyl-methyl, cyclopentyl-ethyl, cyclohexyl-methyl, cyclobut-1-en-1-yl, cyclobut-1-en-3-yl, cyclobuta-1,3-dien-1-yl and the like.

Preferred saturated polycyclic cycloalkyl radicals or cycloalkyl groups according to the invention include, but are not limited to, for example adamantyl groups and the like.

According to the invention, the term “cycloalkyl” also includes cycloalkenyls, i.e., unsaturated cyclic hydrocarbon radicals containing C═C double bonds between two carbon atoms of the ring molecule. In a broader sense, cycloalkenyls are compounds with one, two or more double bond(s), whereby the number of possible, mostly conjugated double bonds in the molecule depends on the ring size.

Typical cycloalkenyls include, but are not limited to, cyclopropenyl, cyclopentenyl, cyclohexenyl, cyclopentadienyl and the like.

According to the invention, the term “cycloalkyl” also includes cycloalkynyls, i.e., unsaturated cyclic hydrocarbon radicals containing —C═C triple bonds between two carbon atoms of the ring molecule, the triple bond being dependent on the ring size for reasons of ring tension.

Typical cycloalkynes include cyclooctyne.

The cycloalkyl residue or the cycloalkyl group can be attached to the remainder of the molecule of formula (I) and/or formula (II) via any suitable C atom.

The cycloalkyl residue or the cycloalkyl group, as defined above, may also be substituted.

The term “aryl” alone or as part of another substituent according to the present invention refers to a monovalent aromatic hydrocarbon radical derived by removing a hydrogen atom from a single carbon atom of an aromatic ring system.

In a preferred variant, the term “aryl” comprises a three- to ten-membered monocyclic aryl radical or aryl group or a nine- to twelve-membered polycyclic aryl radical or aryl group. In other still more preferred variants, the carboaryl moiety comprises a three-, four-, five-, six- or seven-membered monocyclic carboaryl moiety or a nine- to twelve-membered bicyclic carboaryl moiety.

In a preferred variant according to the present invention, the aryl radical comprises 3 to 20 carbon atoms. In an even more preferred variant, an aryl radical comprises 3 to 15 carbon atoms. In a most preferred variant, an aryl radical comprises 3 to 10 carbon atoms. Most preferred according to the invention are monocyclic C₃- to C₁₂-aryl groups. Most preferred are monocyclic C₃- to C₇-aryl groups.

Typical aryl radicals include, but are not limited to, benzene, phenyl, biphenyl, naphthyl such as 1- or 2-naphthyl, tetrahydronaphthyl, fluorenyl, indenyl and phenanthrenyl. Typical carboaryl radicals further include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, S-indacene, indane, indene, naphthalene, octacene, octaphene, octalene, ovalene, penta-2,4-diene, pentacene, pentalene, pentaphene, perylene, phenalene, phenanthrene, picene, pleiades, pyrene, pyranthrene, rubicene, triphenylene, trinaphthalene and the like.

Preferred aromatic polycyclic aryl radicals or aryl groups according to the invention include, but are not limited to, naphthalene, biphenyl and the like.

The aryl residue or the aryl group can be bonded to the rest of the molecule of formulae (I) to (VIII) and (Va) to (VIIIa) via any suitable C atom.

The aryl residue or the aryl group, as defined above, can also be substituted. For example, the aryl residue forms an anisole group.

The term “arylalkyl” alone or as part of another substituent according to the present invention refers to an acyclic alkyl group in which one of the hydrogen atoms attached to a carbon atom, typically a terminal or sp carbon atom, is replaced by an aryl group as defined herein. In other words, arylalkyl may also be considered as alkyl substituted by aryl. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like.

The term “heteroarylalkyl” alone or as part of another substituent refers to a cyclic alkyl group in which one or more of the hydrogen atom(s) attached to a carbon atom is replaced by a heteroaryl group.

In a preferred embodiment according to the present invention, the heteroarylalkyl group is a 6- to 20-membered heteroarylalkyl, e.g., the alkanyl, alkenyl or alkynyl group of the heteroarylalkyl is a C₁- to C₆-alkyl and the heteroaryl group is a 5- to 15-membered heteroaryl group. In other embodiments, the heteroarylalkyl is a 6- to 13-membered heteroarylalkyl, e.g., the alkanyl, alkenyl or alkynyl group is a C₁- to C₃-alkyl and the heteroaryl group is a 5- to 10-membered heteroaryl.

The term “heterocycloalkyl” alone or as part of another substituent according to the present invention refers to a saturated, non-aromatic, cyclic monovalent hydrocarbon radical in which one or more carbon atom(s) is/are independently replaced by the same or a different heteroatom. Typical heteroatoms for replacing the carbon atom(s) include, but are not limited to, N, P, O, S, Si, etc. Typical heterocycloalkyl groups include, but are not limited to, groups derived from epoxides, azirines, thiiranes, imidazolidine, morpholine, piperazine, piperidine, pyrazolidine, pyrrolidone, quinuclidine and the like.

The heterocycloalkyl residue can occur as a monocyclic compound, which has only a single ring, or as a polycyclic compound, which has two or more rings.

Preferably, the term “heterocycloalkyl” comprises three- to seven-membered, saturated or mono- or polyunsaturated heterocycloalkyl radicals comprising one, two, three or four heteroatoms selected from the group consisting of O, N and S. The heteroatom or heteroatoms may occupy any position in the heterocycloalkyl ring. The heteroatom or heteroatoms may occupy any position in the heterocycloalkyl ring.

In a preferred variant, the term “heterocycloalkyl” comprises a three- to ten-membered monocyclic heterocycloalkyl radical or a nine- to twelve-membered polycyclic heterocycloalkyl radical. In other still more preferred variants, the heterocycloalkyl moiety comprises a three-, four-, five-, six- or seven-membered monocyclic heterocycloalkyl moiety or a nine- to twelve-membered bicyclic heterocycloalkyl moiety.

In a preferred variant according to the present invention, the “heterocycloalkyl” residue or heterocycloalkyl group comprises 3 to 20 ring atoms. In a preferred variant, the heterocycloalkyl moiety comprises 3 to 15 ring atoms. In an even more preferred variant, the heterocycloalkyl moiety comprises 3 to 10 carbon atoms. Most preferred according to the invention are monocyclic heterocycloalkyl radicals comprising 3 to 12 carbon atoms. Most preferred are monocyclic heterocycloalkyl radicals with 5 to 7 ring atoms.

Typical heterocycloalkyl radicals include, but are not limited to: Three to six membered saturated heterocycloalkyl containing one or two nitrogen atoms and/or one oxygen or sulfur atom or one or two oxygen and/or sulfur atoms as ring members comprising aziridinyl, oxiranyl, thiiranyl, azetidinyl, oxetanyl, thietanyl, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-Isoxazolidinyl, 4-Isoxazolidinyl, 5-Isoxazolidinyl, 3-Isothiazolidinyl, 4-Isothiazolidinyl, 5-Isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-Imidazolidinyl, 4-Imidazolidinyl, 2-pyrrolin-2-yl, 2-pyrrolin-3-yl, 3-pyrrolin-2-yl, 3-pyrrolin-3-yl, 1-piperidinyl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl and the like.

The heterocycloalkyl radical or the heterocycloalkyl group, as defined above, may furthermore be substituted.

The heterocycloalkyl radical or the heterocycloalkyl group may be bonded to the remainder of the molecule of formulae (I) to (VIII) and (Va) and (VIII) via a ring carbon atom or a ring heteroatom.

The term “heteroaryl” by itself or as part of another substituent according to the present invention refers to a monovalent heteroaromatic radical obtained by removing a hydrogen atom from a single atom of a heteroaromatic ring system. Typical heteroaryl radicals or Heteroaryl groups include, but are not limited to, those groups derived from acridine, β-carboline, chroman, chromium, cinnoline, furan, imidazole, indazole, indole, indoline, indolizine, isobenzofuran, isochrome, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, thiazole, thiophene, triazole, xanthene and the like.

The heteroaryl residue can occur as a monocyclic compound, which has only a single ring, or as a polycyclic compound, which has two or more rings.

In a preferred variant, the term “heteroaryl” comprises a three- to ten-membered monocyclic heteroaryl radical or a nine- to twelve-membered polycyclic heteroaryl radical. In other still more preferred variants, the heteroaryl moiety comprises a three-, four-, five-, six- or seven-membered monocyclic heteroaryl moiety or a nine- to twelve-membered bicyclic heteroaryl moiety.

Preferably, the term “heteroaryl” comprises three- to seven-membered monocyclic heteroaryl radicals comprising one, two, three or four heteroatoms selected from the group consisting of O, N and S. The heteroatom or heteroatoms may occupy any position in the heteroaryl ring. The heteroatom or heteroatoms may occupy any position in the heteroaryl ring.

In a preferred variant according to the present invention, the heteroaryl moiety or group comprises 3 to 20 ring atoms. In an even more preferred variant, the heteroaryl moiety comprises 6 to 15 ring atoms. In a most preferred variant, the heteroaryl group comprises 6 to 10 ring atoms. Most preferred according to the invention are monocyclic C₃- to C₇-heteroaryl groups.

Particularly preferred heteroaryl radicals or heteroaryl groups include, but are not limited to, those derived from furan, thiophene, pyrrole, benzothiophene, benzofuran, benzimidazole, indole, pyridine, pyrazole, quinoline, imidazole, oxazole, isoxazole and pyrazine.

Three-membered aromatic heteroaryl radicals containing, in addition to carbon atoms, a nitrogen or a sulfur or an oxygen atom as ring atoms include azirinyl, oxirenyl or thiirenyl.

Four-membered aromatic heteroaryl radicals containing, in addition to carbon atoms, a nitrogen or a sulphur or an oxygen atom as ring atoms include acetyl, oxetium ion or thietium ion.

Five-membered aromatic heteroaryl radicals containing, in addition to carbon atoms, one, two or three nitrogen atoms or one or two nitrogen atoms and one sulfur or oxygen atom as ring atoms include 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-Imidazolyl, 4-Imidazolyl, and 1,3,4-triazol-2-yl.

Five-membered aromatic heteroaryl radicals containing one, two, three or four nitrogen atoms as ring atoms include 1-, 2- or 3-pyrrolyl, 1-, 3- or 4-pyrazolyl, 1-, 2- or 4-Imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl, [2H]-tetrazol-5-yl and the like.

Five-membered aromatic heteroaryl radicals containing a heteroatom selected from oxygen or sulfur and optionally one, two or three nitrogen atoms as ring atoms include 2-furyl, 3-furyl, 2-thienyl, 3-thienyl, 3- or 4-isoxazolyl, 3- or 4-isothiazolyl, 2-, 4- or 5-oxazolyl, 2-, 4- or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl.

Six-membered heteroaryl radicals containing, in addition to carbon atoms, one or two or one, two or three nitrogen atoms as ring atoms, and comprising, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.

The heteroaryl residue or the heteroaryl group, as defined above, may also be substituted.

The heteroaryl radical or the heteroaryl group may be bonded to the remainder of the molecule of formula (I) to formula (VIII) and (Va) and (VIII) via a ring carbon atom or a ring heteroatom.

Of the monocyclic heteroaryl radicals mentioned above, particularly preferred in the context of the present invention are those heteroaryl radicals which are derived from the five- or six-membered saturated compounds comprising pyrrolidone, tetrahydrofuran, tetrahydrothiophene, piperidine, tetrahydropyran, tetrahydrothipyran or from the five- or six-membered aromatic compounds comprising pyrrole, furan, thiophene, pyridine, pyrylium ion and thiopyrylium ion, pyrazole, imidazole, imidazoline, pyrimidine, oxazole, thiazole and 1,4-thiazine.

The term “arylalkyl” alone or as part of another substituent according to the present invention refers to an acyclic alkyl group in which one of the hydrogen atoms attached to a carbon atom, typically a terminal or sp carbon atom, is replaced by an aryl group as defined herein. In other words, arylalkyl may also be considered as alkyl substituted by aryl. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like.

The term “heteroarylalkyl” alone or as part of another substituent refers to a cyclic alkyl group in which one of the hydrogen atoms bonded to a carbon atom is replaced by a heteroaryl group. In a preferred embodiment according to the present invention, the heteroarylalkyl group is a 6- to 20-membered heteroarylalkyl, e.g., the alkanyl, alkenyl or alkynyl group of the heteroarylalkyl is a C₁- to C₆-alkyl and the heteroaryl group is a 5- to 15-membered heteroaryl group. In other embodiments, the heteroarylalkyl is a 6- to 13-membered heteroarylalkyl, e.g., the alkanyl, alkenyl or alkynyl group is a C₁- to C₃-alkyl and the heteroaryl group is a 5- to 10-membered heteroaryl.

The term “substituted” in the context of the present invention means that one or more hydrogen atoms of the specified radical or the specified radical are independently replaced by the same or another substituent.

Substituents or substituent groups useful for substituting saturated carbon atoms in the specified group or moiety include, but are not limited to, —X, halo, ═O, —OY, —SiR₃, —SY, ═S, —NZZ, ═NY, ═N—OY, trihalomethyl, —CF₃, —CN, —OCN, —SCN, —NO, —NO₂, ═N₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y or —NYC(NY)NZZ; wherein X is selected from the group consisting of optionally substituted alkyl radical, in particular optionally substituted C₁- to C₁₀-alkyl group, in particular optionally substituted C₁- to C₆-alkyl radical, in particular optionally substituted C₁-, C₂-, C₃- or C₄-alkyl group, optionally substituted alkoxy radical, in particular optionally substituted C₁- to C₆-alkoxy radical, in particular optionally substituted C₁-, C₂-, C₃- or C₄-alkoxy group, optionally substituted alkylthio radical, in particular optionally substituted C₁- to C₆-alkylthio radical, in particular optionally substituted C₁-, C₂-, C₃- or C₄-alkylthio group, optionally substituted cycloalkyl radical, optionally substituted aryl radical, optionally substituted carboaryl radical, optionally substituted carboarylalkyl radical, optionally substituted heteroalkyl radical, optionally substituted heterocycloalkyl radical, optionally substituted heteroaryl radical and optionally substituted heteroarylalkyl radical, and as defined above; and/or
Y means hydrogen or X; and/or
Z is Y or alternatively two Z's together with the nitrogen atom to which they are attached form a four, five, six or seven membered heterocycloalkyl or heteroaryl ring, wherein the heterocycloalkyl or heteroaryl ring may comprise one, two, three or four identical or different heteroatoms selected from the group consisting of nitrogen, oxygen and sulfur.

Specific preferred examples of a substitution are: OH, methyl, ethyl, methoxy, ethoxy, phenyl, which in turn may be substituted with OH, methyl, ethyl, methoxy, ethoxy or CH₃—C(O)— or thiophene.

In a further variant, the one or more substituent group(s), preferably phenyl groups, together with the atoms to which they are attached may form a cyclic ring, including cycloalkyl, cycloalkenyl, heterocycloalkyl or heterocycloalkenyl.

Similarly, substituent groups useful for substituting unsaturated carbon atoms in the specified group or radical include, but are not limited to, —X, halo, ═O, —OY, —SiR₃, —SY, ═S, —NZZ, ═NY, ═N—OY, trihalomethyl, —CF₃, —CN, —OCN, —SCN, —NO, —NO₂, ═N₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ, where X, Y and Z have the same meaning as defined above.

substituents or substituent groups for the substitution of nitrogen atoms in heteroalkyl and heterocycloalkyl radicals, without being limited thereto, —X, —OY, —SiR₃, —SY, —NZZ, trihalomethyl, —CF₃, —CN, —OCN, —SCN, —NO, —NO₂, ═N₂, —N₃, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ, where X, Y and Z also have the same meaning as defined above.

The term “substituted” specifically provides for one or more, i.e., two, three, four, five, six or more, substitutions that are common in the art. However, it is generally recognized by those skilled in the art that the substituents should be selected so as not to adversely affect the useful properties of the compound or its function.

Suitable substituents within the scope of the present invention preferably include halogen groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy groups or heteroaryloxy groups, arylalkyl or heteroarylalkyl groups, arylalkoxy or heteroarylalkoxy groups, amino groups, alkyl and dialkylamino groups, carbamoyl groups, alkylcarbonyl groups, carboxyl groups, alkoxycarbonyl groups, alkylaminocarbonyl groups, dialkylaminocarbonyl groups, arylcarbonyl groups, aryloxycarbonyl groups, alkylsulphonyl groups, arylsulphonyl groups, cycloalkyl groups, cyano groups, C₁- to C₆-alkylthio groups, arylthio groups, nitro groups, keto groups, acyl groups, boronate or boronyl groups, phosphate or phosphonyl groups, sulphamyl groups, sulphonyl groups, sulphinyl groups and combinations thereof. In the case of substituted combinations such as substituted arylalkyl, either the aryl or the alkyl group may be substituted, or both the aryl and the alkyl group may be substituted with one or more substituents.

Preferred substituents for the above-mentioned groups or radicals are selected in particular from COOH, COO-alkyl, NH₂, NO₂, OH, SH, CN, Si, halogens, linear or branched C₁- to C₆-alkyl groups, linear or branched C₁- to C₆-alkoxy groups or linear or branched C₁- to C₆-alkylthio groups, wherein one or more H atoms in the alkyl groups may be replaced by halogen.

In addition, in some cases suitable substituents may be combined to form one or more rings as known to those skilled in the art.

The term “optionally substituted” in the context of the present invention denotes the presence or absence of the substituent group(s), i.e., means “substituted” or “unsubstituted”. For example, the term “optionally substituted alkyl” includes both unsubstituted alkyl and substituted alkyl.

According to the invention, the substituents used to replace a particular radical or radical may in turn be further substituted, typically with one or more identical or different radicals selected from the various groups indicated above and as defined in detail above.

In a particularly preferred variant according to the first aspect of the present invention, the radicals R1 and R2 in the general formulae (I) to (VIII) and (Va) and (VIII) are identical or different.

Preferably, R1 in the general formulae (I) to (VIII) and (Va) to (VIIIa) represents H or an optionally substituted alkyl group or an optionally substituted phenyl group or an optionally substituted thiophene group.

Further preferably R2 in the general formulae (I) to (VIII) and (Va) to (Villa) represents H or an optionally substituted alkyl group or an optionally substituted phenyl group or an optionally substituted thiophene group.

Coolants with particularly advantageous properties, i.e., a particularly intensive and effective and preferably simultaneously long-lasting cooling effect, are regularly found in structures in which, in the general formulae (I), (V) or (Va), which have a triazine ring in their basic structure, at least one of the radicals R1 and R2 stands for an optionally substituted phenyl group or an optionally substituted thiophene group.

Even more preferably, at least one of the radicals R1 and R2 is an optionally substituted phenyl group.

Most preferred are compounds of the general formulae (I), (V) or (Va), in which both R1 and R2 represent an optionally substituted phenyl group.

Specific preferred examples of a substitution of the phenyl group are: OH, methyl, ethyl, methoxy or ethoxy.

Most preferably, R1 and R2 represent a phenyl group.

In an even more preferred variant, the two phenyl groups, together with the atoms to which they are attached, form a cyclic ring, including cycloalkyl or heterocycloalkyl.

The most preferred compounds are those of the general formulae (I), (V) or (Va), in which both R1 and R2 are a phenyl group which is not substituted.

In a further preferred variant, in the compounds of the general formulae (I), (V) or (Va) R1 and R2 are each a substituted or unsubstituted phenyl group which, together with the C atoms of the core structure to which they are bonded, form a conjugated or non-conjugated ring system.

It has been shown that in particular the compounds of the general formulae (I), (V) or (Va) described above, in which both R1 and R2 are a substituted or unsubstituted phenyl group, exhibit excellent TRPM8 activities and are capable of producing extraordinarily intense sensory cooling effects even when used in small quantities.

Coolants with particularly advantageous properties, i.e., a particularly intensive and effective and preferably simultaneously long-lasting cooling effect, are also found in structures in which in the general formulae (II), (VI) or (VIa), which have a pyrazine ring in their basic structure, at least one of the radicals R1 and R2 stands for an optionally substituted phenyl group or an optionally substituted thiophene group.

Even more preferably, at least one of the radicals R1 and R2 is an optionally substituted phenyl group

Most preferred are compounds of the general formulae (II), (VI) or (VIa), in which both R1 and R2 represent an optionally substituted phenyl group.

Specific preferred examples of a substitution are: OH, methyl, ethyl, methoxy or ethoxy.

Most preferably, R1 and R2 represent a phenyl group.

In an even more preferred variant, the two phenyl groups, together with the atoms to which they are attached, form a cyclic ring, including cycloalkyl or heterocycloalkyl.

The most preferred compounds are those of the general formulae (II), (VI) or (VIa) in which both R1 and R2 are a phenyl group which is not substituted.

In a further preferred variant, in the compounds of the general formulae (II), (VI) or (VIa), R1 and R2 are each a substituted or unsubstituted phenyl group which, together with the C atoms of the core structure to which they are bonded, form a conjugated or non-conjugated ring system.

It has been shown that compounds of the general formulae (I), (V) or (Va) in particular, in which both R1 and R2 are a substituted or unsubstituted phenyl group, exhibit excellent TRPM8 activities and are capable of producing extraordinarily intense sensory cooling effects even when used in small quantities.

Coolants with particularly advantageous properties, i.e. a particularly intensive and effective and preferably simultaneously long-lasting cooling effect and/or optionally a particularly efficient masking of undesirable taste impressions, are also found in structures in which, in the general formulae (III), (VII) or (VIIa), which have an oxazole ring in their basic structure, at least one of the radicals R1 and R2 stands for an optionally substituted phenyl group or an optionally substituted thiophene group.

Even more preferably, at least one of the radicals R1 and R2 is an optionally substituted phenyl group

Most preferred are compounds of the general formulae (III), (VII) or (VIIa), in which both R1 and R2 represent an optionally substituted phenyl group.

Specific preferred examples of a substitution are: OH, methyl, ethyl, methoxy or ethoxy.

Most preferably, R1 and R2 represent a phenyl group.

In an even more preferred variant, the two phenyl groups, together with the atoms to which they are attached, form a cyclic ring, including cycloalkyl or heterocycloalkyl.

The most preferred compounds are those of the general formulae (III), (VII) or (VIIa) in which both R1 and R2 are a phenyl group which is not substituted.

In a further preferred variant, in the compounds of the general formulae (III), (VII) or (VIIa), R1 and R2 are each a substituted or unsubstituted phenyl group which, together with the C atoms of the core structure to which they are bonded, form a conjugated or non-conjugated ring system.

It has been shown that in particular these compounds of the general formulae (III), (VII) or (VIIa), in which both R1 and R2 are a substituted or unsubstituted phenyl group, exhibit excellent TRPM8 activities and are capable of producing extraordinarily intense sensory cooling effects even in small quantities.

Coolants with particularly advantageous properties, i.e., a particularly intensive and effective and preferably simultaneously long-lasting cooling effect, are also found in structures in which in the general formulae (IV), (VIII) or (Villa), which have an imidazole ring in their basic structure, at least one of the radicals R1 and R2 stands for an optionally substituted phenyl group or an optionally substituted thiophene group.

Even more preferably, at least one of the radicals R1 and R2 is an optionally substituted phenyl group

Most preferred are compounds of the general formulae (IV), (VIII) and (Villa), in which both R1 and R2 represent an optionally substituted phenyl group.

Specific preferred examples of a substitution are: OH, methyl, ethyl, methoxy or ethoxy.

Most preferably, R1 and R2 represent a phenyl group.

In an even more preferred variant, the two phenyl groups, together with the atoms to which they are attached, form a cyclic ring, including cycloalkyl or heterocycloalkyl.

The most preferred compounds are those of the general formulae (IV), (VIII) or (Villa) in which both R1 and R2 are a phenyl group which is not substituted.

In a further preferred variant, in the compounds of the general formulae (IV), (VIII) or (Villa), R1 and R2 are each a substituted or unsubstituted phenyl group which, together with the C atoms of the core structure to which they are bonded, form a conjugated or non-conjugated ring system.

It has been shown that in particular these compounds of the general formulae (IV), (VIII) or (VIIIa), in which both R1 and R2 are a substituted or unsubstituted phenyl group, exhibit excellent TRPM8 activities and are capable of producing extraordinarily intense sensory cooling effects even in small quantities.

Also preferred according to the invention are coolants of the general formulae (I) to (VIII) in which, in addition, Y represents a linear alkylene group, an optionally branched alkylene group, an alkylaryl group or an alkylheteroaryl group. Preferably, the alkylene group is a methylene group —CH₂—, an ethylene group —CH—CH₂₂— or a propylene group —CH—CH—CH₂₂₂—. Alternatively, Y in the general formulae (I) to (VIII) also represents a branched alkylene group, preferably a methylene group, which is substituted by a methyl group, an ethyl group, a linear or branched propyl group or a linear or branched butyl group.

Even more preferred according to the invention are compounds of the general formulae (I) to (VIII) in which Y represents a methylene group or a methylene group which is substituted by a methyl group, an ethyl group, a linear or branched propyl group or a linear or branched butyl group. Such compounds have particularly pronounced cooling attributes, as described below.

Even more preferred are coolants of the general formulae (I) or (V) which have one of the following structural combinations:
R1 and/or R2=optionally substituted phenyl group and Y=methylene;
R1 and/or R2=optionally substituted phenyl group and Y=linear ethylene; or
R1 and/or R2=optionally substituted phenyl group and Y=linear or branched propylene; or
R1 and/or R2=optionally substituted phenyl group and Y=methylene substituted with a methyl group, an ethyl group, a linear or branched propyl group or a linear or branched butyl group.

Also preferred are coolants of the general formulae (II) or (VI) which have one of the following structural combinations:
R1 and/or R2=optionally substituted phenyl group and Y=methylene;
R1 and/or R2=optionally substituted phenyl group and Y=linear ethylene; or
R1 and/or R2=optionally substituted phenyl group and Y=linear or branched propylene; or
R1 and/or R2=optionally substituted phenyl group and Y=methylene substituted with a methyl group, an ethyl group, a linear or branched propyl group or a linear or branched butyl group.

Further preferred are those coolants of the general formulae (III) or (VII) which have one of the following structural combinations:
R1 and/or R2=optionally substituted phenyl group and Y=methylene;
R1 and/or R2=optionally substituted phenyl group and Y=linear ethylene; or
R1 and/or R2=optionally substituted phenyl group and Y=linear or branched propylene; or
R1 and/or R2=optionally substituted phenyl group and Y=methylene substituted with a methyl group, an ethyl group, a linear or branched propyl group or a linear or branched butyl group.

Further preferred are those coolants of the general formulae (IV) or (VIII) which have one of the following structural combinations:
R1 and/or R2=optionally substituted phenyl group and Y=methylene;
R1 and/or R2=optionally substituted phenyl group and Y=linear ethylene; or
R1 and/or R2=optionally substituted phenyl group and Y=linear or branched propylene; or
R1 and/or R2=optionally substituted phenyl group and Y=methylene substituted with a methyl group, an ethyl group, a linear or branched propyl group or a linear or branched butyl group.

Even more preferred are those coolants of the general formulae (I) or (V) which have one of the following structural combinations:
R1 and/or R2=phenyl group and Y=methylene;
R1 and/or R2=phenyl group and Y=linear ethylene; or
R1 and/or R2=phenyl group and Y=linear or branched propylene; or
R1 and/or R2=phenyl group and Y=methylene substituted with a methyl group, an ethyl group, a linear or branched propyl group or a linear or branched butyl group.

Also preferred are coolants of the general formulae (II) or (VI) which have one of the following structural combinations:
R1 and/or R2=phenyl group and Y=methylene;
R1 and/or R2=phenyl group and Y=linear ethylene; or
R1 and/or R2=phenyl group and Y=linear or branched propylene; or
R1 and/or R2=phenyl group and Y=methylene substituted with a methyl group, an ethyl group, a linear or branched propyl group or a linear or branched butyl group.

Further preferred are coolants of the general formulae (III) or (VII) which have one of the following structures:
R1 and/or R2=phenyl group and Y=methylene;
R1 and/or R2=phenyl group and Y=linear ethylene; or
R1 and/or R2=phenyl group and Y=linear or branched propylene; or
R1 and/or R2=phenyl group and Y=methylene substituted with a methyl group, an ethyl group, a linear or branched propyl group or a linear or branched butyl group.

Further preferred are coolants of the general formulae (IV) or (VIII) which have one of the following structures:
R1 and/or R2=phenyl group and Y=methylene;
R1 and/or R2=phenyl group and Y=linear ethylene; or
R1 and/or R2=phenyl group and Y=linear or branched propylene; or
R1 and/or R2=phenyl group and Y=methylene substituted with a methyl group, an ethyl group, a linear or branched propyl group or a linear or branched butyl group.

Of the last-mentioned coolants according to the general formulae (I) to (VIII), those compounds in which Y represents a methylene group substituted by a methyl group or an ethyl group are most preferred.

Such compounds are characterized by particularly intensive cooling effects.

In the general formulae (I) to (VIII), Z is selected from the group consisting of NH₂, an NHRa group, an NRaRb group, an optionally substituted linear or branched alkyl group, an optionally substituted linear or branched alkenyl group, an optionally substituted linear or branched alkylthio group an optionally substituted linear or branched alkoxy group, OH, an optionally substituted cycloalkyl group, an optionally substituted heterocycloalkyl group, an optionally substituted aryl group or an optionally substituted heteroaryl group, wherein Ra and/or Rb is/are
is an optionally substituted linear or branched alkyl group, or
is an optionally substituted linear or branched alkenyl group, or
is an optionally substituted linear or branched alkenyl group, or
is an optionally substituted linear or branched alkynyl group, or
is an optionally substituted linear or branched alkoxy group, or
an optionally substituted linear or branched alkylthio group, is
or an optionally substituted acyl group is R—(C═O)—, or
is an optionally substituted cycloalkyl group, or
is an optionally substituted aryl group, or
is an optionally substituted heterocycloalkyl group, or
is an optionally substituted heteroaryl group.

In a further alternative variant, the Ra and Rb radicals of the NRaRb group as defined above are linked and form a saturated or unsaturated ring, preferably a saturated or unsaturated three- to eight-membered ring.

Particularly preferred according to the invention are coolants of the general formulae (I) to (VIII) in which Ra and/or Rb in the NHRa group or the NRaRb group represents a C₁ to C₃ alkyl group, preferably a methyl group.

Even more preferred according to the invention are those coolants of the general formulae (I) to (VIII) in which Z is selected from the group consisting of:
—NH₂, —NH—CH₃,
—NH—CH₂—CH₃, —NH—CH₂—CH₂—CH₃,
—NH—CH₂—CH₂—CH₂—CH₃, —NH—CH(CH₃)—CH(CH₃)₂,
—NH—CH(CH₃)—CH₂—CH₂—CH₃, —NH—CH₂—CH(CH₃)₂,
—NH—CH₂—CH₂—O—CH₃, —NH—CH(CH₃)—CH₂—O—CH₃,
—NH—C(═O)—CH₃, —NH—C(═O)—O—CH₃,
—NH—CH(CH₃)—CH₂—OH, —NH—CH₂-furanyl,
—NH—CH₂-tetrahydrofuranyl, —NH—CH₂-thiophenyl,
—NH-toluolyl, —NH—CH—(CH₃)₂,
—NH—C(CH₃)₃, —NH-cyclopropyl,
—NH-cyclobutyl, —NH-cyclopentyl,
N(CH3)₂, —N(CH₃)-cyclohexyl,
—N(CH₂—CH₃)₂, azetidinyl,
pyrrolidinyl, piperidinyl,
azacyclobutadienyl, pyrrolyl,
pyridinyl, —O,
—OH, —O—CH₃,
O—C(═O)—CH₃, oxetanyl,
—CH₃, —CH₂—CH₃,
—CH(CH₃)₂, —C(OH)—CH₂—OH,
cyclopropyl, phenyl, and
—CH₂—S—CH₃.

In a preferred variant according to the first aspect of the present invention, therefore, are coolants of the general formulae (I) to (VIII) which have the following structures:

| R1 and/or R2 | Y | Z |
|---|---|---|
| Phenyl substituted if | Methylene or | —NH₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—CH₂—CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂(CH₃)—CH₂—CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH(CH₃)—CH₂—CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH(CH₃)—CH(CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH(CH₃)—CH₂—CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—CH(CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—CH₂—O—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH(CH₃)—CH₂—O—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—C(═O)—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—C(═O)—O—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH(CH₃)—CH₂—OH |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—Furanyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—tetrahydrofuranyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—thiophenyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—toluolyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH—(CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—C(CH₃)₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—cyclopropyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—cyclobutyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—cyclopentyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —N(CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —N(CH₃)—cyclohexyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —N(CH₂—CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Azetidinyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Pyrrolidinyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Piperidinyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Azacyclobutadienyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Pyrrolyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Pyridinyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —O |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —OH |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —O—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —O—C(═O)—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Oxetanyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —CH₂—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —CH(CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —C(OH)—CH₂—OH |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Cyclopropyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Phenyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —CH₂—S—CH₃ |
| necessary | Methylene substituted with a | |
| | methyl group, an ethyl group, a | |
| | linear or branched propyl | |
| | group or a linear or branched | |
| | butyl group | |

Coolants of the general formulae (I), (II), (V) or (VI) which have the following structures are even more preferred according to the invention:

| R1 and/or R2 | Y | Z |
|---|---|---|
| Phenyl substituted if | Methylene or | —NH—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—cyclopropyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —N(CH₃)₂ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Azetidinyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | Pyrrolidinyl |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—CH₂—CH₂—O—CH₃ |
| necessary | Methylene substituted with a | |
| | methyl group, an ethyl group, a | |
| | linear or branched propyl | |
| | group or a linear or branched | |
| | butyl group | |
| Phenyl substituted if | Methylene or | —NH—cyclopentyl |
| necessary | Methylene substituted with a | |
| | methyl group, an ethyl group, a | |
| | linear or branched propyl | |
| | group or a linear or branched | |
| | butyl group | |
| | Methylene or | |
| | Methylene substituted with a | |
| | methyl group, an ethyl group, a | |
| | linear or branched propyl | |
| | group or a linear or branched | |
| | butyl group | |
| Substituted phenyl, if | Methylene or | —NH—CH₂—Furanyl |
| necessary | Methylene substituted with a | |
| | methyl group, an ethyl group, a | |
| | linear or branched propyl | |
| | group or a linear or branched | |
| | butyl group | |

Even more preferred according to the invention are coolants of the general formulae (III), (IV), (VII) or (VIII) which have the following structures:

| R1 and/or R2 | Y | Z |
|---|---|---|
| Phenyl substituted if | Methylene or | —NH—CH₃ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —NH—cyclopropyl |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —NH—CH₂—CH₃ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —CH₂—S—CH₃ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —NH—CH₂—CH₂—CH₃ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —NH—CH(CH₃)₂ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —NH₂ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —N(CH₃)₂ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —NH—C(═O)—CH₃ |
| necessary | Methylene substituted with a | |
| | methyl group, an ethyl group, a | |
| | linear or branched propyl group | |
| | or a linear or branched butyl | |
| | group | |
| Phenyl substituted if | Methylene or | —N—CH(CH₃)—CH₂—OH |
| necessary | Methylene substituted with a | |
| | methyl group, an ethyl group, a | |
| | linear or branched propyl group | |
| | or a linear or branched butyl | |
| | group | |

Also preferred with regard to the TRPM8 activity determined are compounds of the general formulae (I) to (VIII) in which n and m are each 1.

Of the physiological coolants defined and described above, those in which X stands for a sulfur atom in the general formulae (I) to (IV) are particularly preferred. Such compounds have particularly pronounced cooling attributes, as described below.

Particularly advantageous cooling attributes are surprisingly exhibited by compounds according to the present invention in which, in the general formulae (I) to (VIII), R1 and R2 each represent an unsubstituted phenyl group, X represents a sulfur atom, Y represents a methylene group or a methylene group substituted with a methyl group, an ethyl group, a linear or branched propyl group or a linear or branched butyl group, and Z has the meaning as defined above.

Such compounds are particularly efficient cooling substances, as illustrated below.

Most preferred according to the invention are compounds of the general formulae (Va) to (Villa) in which the radicals R1 and/or R2 represent an unsubstituted phenyl group or a substituted phenyl group, Y represents a branched alkylene group, preferably a methylene group, which is substituted by a methyl group, an ethyl group, a linear or branched propyl group or a linear or branched butyl group, and Z has the meaning as defined above for the compounds of the general formulae (I) to (VIII).

Such compounds exhibit pronounced TRPM8 activities and are extremely intensive sensory coolants even in small quantities.

Coolants of the general formulae (Va) or (VIa) which have one of the following structural combinations are particularly preferred:
R1 and/or R2=optionally substituted phenyl group and Y=methylene substituted with a methyl group; or
R1 and/or R2=optionally substituted phenyl group and Y=methylene substituted with an ethyl group; or
R1 and/or R2=optionally substituted phenyl group and Y=methylene substituted with a linear or branched propyl group; or
R1 and/or R2=optionally substituted phenyl group and Y=methylene substituted with a linear or branched butyl group.

Particularly preferred are also those coolants of the general formulae (VIIa) or (Villa) which have one of the following structural combinations:
R1 and/or R2=optionally substituted phenyl group and Y=methylene substituted with a methyl group; or
R1 and/or R2=optionally substituted phenyl group and Y=methylene substituted with an ethyl group; or
R1 and/or R2=optionally substituted phenyl group and Y=methylene substituted with a linear or branched propyl group; or
R1 and/or R2=optionally substituted phenyl group and Y=methylene substituted with a linear or branched butyl group.

Also preferred are coolants of the general formulae (Va) or (VIa) which have one of the following structural combinations:
R1 and/or R2=phenyl group and Y=methylene substituted with a methyl group; or
R1 and/or R2=phenyl group and Y=methylene substituted with an ethyl group; or
R1 and/or R2=phenyl group and Y=methylene substituted with a linear or branched propyl group; or
R1 and/or R2=phenyl group and Y=methylene substituted with a linear or branched butyl group.

Also preferred are coolants of the general formulae (VIIa) or (Villa) which have one of the following structural combinations:
R1 and/or R2=phenyl group and Y=methylene substituted with a methyl group; or
R1 and/or R2=phenyl group and Y=methylene substituted with an ethyl group; or
R1 and/or R2=phenyl group and Y=methylene substituted with a linear or branched propyl group; or
R1 and/or R2=phenyl group and Y=methylene substituted with a linear or branched butyl group.

Surprisingly, outstanding cooling attributes are exhibited by compounds according to the present invention in which in the general formulae (Va) to (Villa) R1 and R2 each stand for an unsubstituted phenyl group, Y stands for a branched alkylene group, preferably for a methylene group which is substituted by a methyl group, an ethyl group, a linear or branched propyl group or a linear or branched butyl group and Z has the meaning as defined above for the compounds of the general formulae (I) to (VIII).

Most preferred are therefore coolants of the general formulae (Va) or (VIa), which have one of the following structural combinations:
R1 and R2=phenyl group and Y=methylene substituted with a methyl group; or
R1 and R2=phenyl group and Y=methylene substituted with an ethyl group; or
R1 and R2=phenyl group and Y=methylene substituted with a linear or branched propyl group; or
R1 and R2=phenyl group and Y=methylene substituted with a linear or branched butyl group; and Coolants of the general formulae (VIIa) or (Villa) which have one of the following structural combinations:
R1 and R2=phenyl group and Y=methylene substituted with a methyl group; or
R1 and R2=phenyl group and Y=methylene substituted with an ethyl group; or
R1 and R2=phenyl group and Y=methylene substituted with a linear or branched propyl group; or
R1 and R2=phenyl group and Y=methylene substituted with a linear or branched butyl group.

Of the last-mentioned coolants according to the general formulae (Va) to (Villa), those compounds in which Y represents a methylene group substituted by a methyl group or an ethyl group are most preferred.

Such compounds have a particularly high TRPM8 activity and are capable of producing extraordinarily intense sensory cooling effects even when used in small quantities.

In the general formulae (Va), (VIa), (VIIa) and (VIIIa), Z is selected from the group consisting of NH₂, an NHRa group, an NRaRb group, an optionally substituted linear or branched alkyl group, an optionally substituted linear or branched alkenyl group, an optionally substituted linear or branched alkylthio group, an optionally substituted linear or branched alkoxy group, OH, an optionally substituted cycloalkyl group, an optionally substituted heterocycloalkyl group, an optionally substituted aryl group, or an optionally substituted heterocycloalkyl group, an optionally substituted linear or branched alkoxy group, OH, an optionally substituted cycloalkyl group, an optionally substituted heterocycloalkyl group, an optionally substituted aryl group or an optionally substituted heteroaryl group, wherein Ra and/or Rb is/are
is an optionally substituted linear or branched alkyl group, or
is an optionally substituted linear or branched alkenyl group, or
is an optionally substituted linear or branched alkenyl group, or
is an optionally substituted linear or branched alkynyl group, or
is an optionally substituted linear or branched alkoxy group, or
an optionally substituted linear or branched alkylthio group, is
or an optionally substituted acyl group is R—(C═O)—, or
is an optionally substituted cycloalkyl group, or
is an optionally substituted aryl group, or
is an optionally substituted heterocycloalkyl group, or
is an optionally substituted heteroaryl group.

In a further alternative variant, the Ra and Rb radicals of the NRaRb group as defined above are linked and form a saturated or unsaturated ring, preferably a saturated or unsaturated three- to eight-membered ring.

Particularly preferred according to the invention are coolants of the general formulae (Va) to (Villa) in which Ra and/or Rb in the NHRa group or the NRaRb group represents a C₁ to C₃ alkyl group, preferably a methyl group.

Even more preferred according to the invention are those coolants of the general formulae (Va), (VIIa) and (VIIIa) in which Z is selected from the group consisting of:
—NH₂, —NH—CH₃,
—NH—CH₂—CH₃, —NH—CH₂—CH₂—CH₃,
—NH—CH₂—CH₂—CH₂—CH₃, —NH—CH(CH₃)—CH(CH₃)₂,
—NH—CH(CH₃)—CH₂—CH₂—CH₃, —NH—CH₂—CH(CH₃)₂,
—NH—CH₂—CH₂—O—CH₃, —NH—CH(CH₃)—CH₂—O—CH₃,
—NH—C(═O)—CH₃, —NH—C(═O)—O—CH₃,
—NH—CH(CH₃)—CH₂—OH, —NH—CH₂-furanyl,
—NH—CH₂-tetrahydrofuranyl, —NH—CH₂-thiophenyl,
—NH-toluolyl, —NH—CH—(CH₃)₂,
—NH—C(CH₃)₃, —NH-cyclopropyl,
—NH-cyclobutyl, —NH-cyclopentyl,
N(CH₃)₂, —N(CH₃)-cyclohexyl,
—N(CH₂—CH₃)₂, azetidinyl,
pyrrolidinyl, piperidinyl,
azacyclobutadienyl, pyrrolyl,
pyridinyl, —O,
—OH, —O—CH₃,
O—C(═O)—CH₃, oxetanyl,
—CH₃, —CH₂—CH₃,
—CH(CH₃)₂, —C(OH)—CH₂—OH,
cyclopropyl, phenyl, and
—CH₂—S—CH₃.

Even more preferred according to the invention are such coolants of the general formula (VIa) in which Z is selected from the group consisting of —NH₂, —NH—CH₃, —NH—CH₂—CH₃, —NH—CH₂—CH₂—CH₃, —NH—CH₂—CH₂—CH₂—CH₃, —NH—CH(CH₃)—CH(CH₃)₂, —NH—CH(CH₃)—CH₂—CH₂—CH₃, —NH—CH₂—CH(CH₃)₂, —NH—CH₂—CH₂—O—CH₃, —NH—CH(CH₃)—CH₂—O—CH₃, —NH—C(═O)— CH₃, —NH—C(═O)—O—CH₃, —NH—CH(CH₃)—CH₂—OH, —NH—CH₂-furanyl, —NH—CH₂-tetrahydrofuranyl, —NH—CH₂-thiophenyl, —NH-toluolyl, —NH—CH—(CH₃)₂, —NH—C(CH₃)₃, —NH-cyclopropyl, —NH— cyclobutyl, —NH-cyclopentyl, —N(CH₃)₂, —N(CH₃)-cyclohexyl, —N(CH₂—CH₃)₂, azetidinyl, pyrrolidinyl, piperidinyl, azacyclobutadienyl, pyrrolyl, pyridinyl, —O, —O—CH₃, —O—C(═O)—CH₃, oxetanyl, —CH₃, —CH₂—CH₃, —CH(CH₃)₂, —C(OH)—CH₂—OH, cyclopropyl, phenyl, and-CH₂—S—CH₃.

In the general formula (VIa), Z does not stand for —NH-phenyl, —N(CH3)-phenyl, —OH, —OC2H5 or —OC(CH3)3.

In a preferred variant according to the first aspect of the present invention, therefore, are coolants of the general formulae (Va) to (Villa) which have the following structures:

| R1 and/or R2 | Y | Z |
|---|---|---|
| Phenyl substituted if | Methylene substituted with a | —NH₂ |
| necessary | methyl group, an ethyl group, a | |
| | linear or branched propyl | |
| | group or a linear or branched | |
| | butyl group | |
| Phenyl substituted if | Methylene substituted with a | —NH—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH₂—CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH₂—CH₂—CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH₂(CH₃)—CH₂—CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH(CH₃)—CH₂—CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH(CH₃)—CH(CH₃)₂ |
| necessary | methyl group, an ethyl group, a | |
| | linear or branched propyl | |
| | group or a linear or branched | |
| | butyl group | |
| Phenyl substituted if | Methylene substituted with a | —NH—CH(CH₃)—CH₂—CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH₂—CH(CH₃)₂ |
| necessary | methyl group, an ethyl group, a | |
| | linear or branched propyl | |
| | group or a linear or branched | |
| | butyl group | |
| Phenyl substituted if | Methylene substituted with a | —NH—CH₂—CH₂—O—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH(CH₃)—CH₂—O—CH₃ |
| necessary | methyl group, an ethyl group, a | |
| | linear or branched propyl | |
| | group or a linear or branched | |
| | butyl group | |
| Phenyl substituted if | Methylene substituted with a | —NH—C(═O)—CH₃ |
| necessary | methyl group, an ethyl group, a | |
| | linear or branched propyl | |
| | group or a linear or branched | |
| | butyl group | |
| Phenyl substituted if | Methylene substituted with a | —NH—C(═O)—O—CH₃ |
| necessary | methyl group, an ethyl group, a | |
| | linear or branched propyl | |
| | group or a linear or branched | |
| | butyl group | |
| Phenyl substituted if | Methylene substituted with a | —NH—CH(CH₃)—CH₂—OH |
| necessary | methyl group, an ethyl group, a | |
| | linear or branched propyl | |
| | group or a linear or branched | |
| | butyl group | |
| Phenyl substituted if | Methylene substituted with a | —NH—CH₂—furanyl |
| necessary | methyl group, an ethyl group, a | |
| | linear or branched propyl | |
| | group or a linear or branched | |
| | butyl group | |
| Phenyl substituted if | Methylene substituted with a | —NH—CH₂—tetrahydrofuranyl |
| necessary | methyl group, an ethyl group, a | |
| | linear or branched propyl | |
| | group or a linear or branched | |
| | butyl group | |
| Phenyl substituted if | Methylene substituted with a | —NH—CH₂—thiophenyl |
| necessary | methyl group, an ethyl group, a | |
| | linear or branched propyl | |
| | group or a linear or branched | |
| | butyl group | |
| Phenyl substituted if | Methylene substituted with a | —NH—toluolyl |
| necessary | methyl group, an ethyl group, a | |
| | linear or branched propyl | |
| | group or a linear or branched | |
| | butyl group | |
| Phenyl substituted if | Methylene substituted with a | —NH—CH—(CH₃)₂ |
| necessary | methyl group, an ethyl group, a | |
| | linear or branched propyl | |
| | group or a linear or branched | |
| | butyl group | |
| Phenyl substituted if | Methylene substituted with a | —NH—C(CH₃)₃ |
| necessary | methyl group, an ethyl group, a | |
| | linear or branched propyl | |
| | group or a linear or branched | |
| | butyl group | |
| Phenyl substituted if | Methylene substituted with a | —NH—cyclopropyl |
| necessary | methyl group, an ethyl group, a | |
| | linear or branched propyl | |
| | group or a linear or branched | |
| | butyl group | |
| Phenyl substituted if | Methylene substituted with a | —NH—cyclobutyl |
| necessary | methyl group, an ethyl group, a | |
| | linear or branched propyl | |
| | group or a linear or branched | |
| | butyl group | |
| Phenyl substituted if | Methylene substituted with a | —NH—cyclopentyl |
| necessary | methyl group, an ethyl group, a | |
| | linear or branched propyl | |
| | group or a linear or branched | |
| | butyl group | |
| Phenyl substituted if | Methylene substituted with a | —N(CH₃)₂ |
| necessary | methyl group, an ethyl group, a | |
| | linear or branched propyl | |
| | group or a linear or branched | |
| | butyl group | |
| Phenyl substituted if | Methylene substituted with a | —N(CH₃)—cyclohexyl |
| necessary | methyl group, an ethyl group, a | |
| | linear or branched propyl | |
| | group or a linear or branched | |
| | butyl group | |
| Phenyl substituted if | Methylene substituted with a | —N(CH₂—CH₃)₂ |
| necessary | methyl group, an ethyl group, a | |
| | linear or branched propyl | |
| | group or a linear or branched | |
| | butyl group | |
| Phenyl substituted if | Methylene substituted with a | azetidinyl |
| necessary | methyl group, an ethyl group, a | |
| | linear or branched propyl | |
| | group or a linear or branched | |
| | butyl group | |
| Phenyl substituted if | Methylene substituted with a | pyrrolidinyl |
| necessary | methyl group, an ethyl group, a | |
| | linear or branched propyl | |
| | group or a linear or branched | |
| | butyl group | |
| Phenyl substituted if | Methylene substituted with a | piperidinyl |
| necessary | methyl group, an ethyl group, a | |
| | linear or branched propyl | |
| | group or a linear or branched | |
| | butyl group | |
| Phenyl substituted if | Methylene substituted with a | azacyclobutadienyl |
| necessary | methyl group, an ethyl group, a | |
| | linear or branched propyl | |
| | group or a linear or branched | |
| | butyl group | |
| Phenyl substituted if | Methylene substituted with a | pyrrolyl |
| necessary | methyl group, an ethyl group, a | |
| | linear or branched propyl | |
| | group or a linear or branched | |
| | butyl group | |
| Phenyl substituted if | Methylene substituted with a | pyridinyl |
| necessary | methyl group, an ethyl group, a | |
| | linear or branched propyl | |
| | group or a linear or branched | |
| | butyl group | |
| Phenyl substituted if | Methylene substituted with a | —O |
| necessary | methyl group, an ethyl group, a | |
| | linear or branched propyl | |
| | group or a linear or branched | |
| | butyl group | |
| Phenyl substituted if | Methylene substituted with a | —OH |
| necessary | methyl group, an ethyl group, a | (except for the general formula |
| | linear or branched propyl | (VIa)) |
| | group or a linear or branched | |
| | butyl group | |
| Phenyl substituted if | Methylene substituted with a | —O—CH₃ |
| necessary | methyl group, an ethyl group, a | |
| | linear or branched propyl | |
| | group or a linear or branched | |
| | butyl group | |
| Phenyl substituted if | Methylene substituted with a | —O—C(═O)—CH₃ |
| necessary | methyl group, an ethyl group, a | |
| | linear or branched propyl | |
| | group or a linear or branched | |
| | butyl group | |
| Phenyl substituted if | Methylene substituted with a | Oxetanyl |
| necessary | methyl group, an ethyl group, a | |
| | linear or branched propyl | |
| | group or a linear or branched | |
| | butyl group | |
| Phenyl substituted if | Methylene substituted with a | —CH₃ |
| necessary | methyl group, an ethyl group, a | |
| | linear or branched propyl | |
| | group or a linear or branched | |
| | butyl group | |
| Phenyl substituted if | Methylene substituted with a | —CH₂—CH₃ |
| necessary | methyl group, an ethyl group, a | |
| | linear or branched propyl | |
| | group or a linear or branched | |
| | butyl group | |
| Phenyl substituted if | Methylene substituted with a | —CH(CH₃)₂ |
| necessary | methyl group, an ethyl group, a | |
| | linear or branched propyl | |
| | group or a linear or branched | |
| | butyl group | |
| Phenyl substituted if | Methylene substituted with a | —C(OH)—CH₂—OH |
| necessary | methyl group, an ethyl group, a | |
| | linear or branched propyl | |
| | group or a linear or branched | |
| | butyl group | |
| Phenyl substituted if | Methylene substituted with a | cyclopropyl |
| necessary | methyl group, an ethyl group, a | |
| | linear or branched propyl | |
| | group or a linear or branched | |
| | butyl group | |
| Phenyl substituted if | Methylene substituted with a | phenyl |
| necessary | methyl group, an ethyl group, a | |
| | linear or branched propyl | |
| | group or a linear or branched | |
| | butyl group | |
| Phenyl substituted if | Methylene substituted with a | —CH₂—S—CH₃ |
| necessary | methyl group, an ethyl group, a | |
| | linear or branched propyl | |
| | group or a linear or branched | |
| | butyl group | |

Coolants of the general formulae (Va) or (VIa) which have the following structures are even more preferred according to the invention:

| R1 and/or R2 | Y | Z |
|---|---|---|
| Phenyl substituted if | Methylene substituted with a | —NH—CH₃ |
| necessary | methyl group, an ethyl group, a | |
| | linear or branched propyl | |
| | group or a linear or branched | |
| | butyl group | |
| Phenyl substituted if | Methylene substituted with a | —NH—cyclopropyl |
| necessary | methyl group, an ethyl group, a | |
| | linear or branched propyl | |
| | group or a linear or branched | |
| | butyl group | |
| Phenyl substituted if | Methylene substituted with a | —N(CH₃)₂ |
| necessary | methyl group, an ethyl group, a | |
| | linear or branched propyl | |
| | group or a linear or branched | |
| | butyl group | |
| Phenyl substituted if | Methylene substituted with a | pyrrolidinyl |
| necessary | methyl group, an ethyl group, a | |
| | linear or branched propyl | |
| | group or a linear or branched | |
| | butyl group | |

Even more preferred according to the invention are also such coolants of the general formulae (VIIa) or (VIIIa) which have the following structures:

| R1 and/or R2 | Y | Z |
|---|---|---|
| Phenyl substituted if | Methylene substituted with a | —NH—CH₃ |
| necessary | methyl group, an ethyl group, a | |
| | linear or branched propyl group | |
| | or a linear or branched butyl | |
| | group | |
| Phenyl substituted if | Methylene substituted with a | —NH—cyclopropyl |
| necessary | methyl group, an ethyl group, a | |
| | linear or branched propyl group | |
| | or a linear or branched butyl | |
| | group | |
| Phenyl substituted if | Methylene substituted with a | —NH—CH₂—CH₂—CH₃ |
| necessary | methyl group, an ethyl group, a | |
| | linear or branched propyl group | |
| | or a linear or branched butyl | |
| | group | |

In addition, compounds of the general formulae (Va) to (Villa), in which m is 1 in each case, appear to be preferred with regard to the TRPM8 activity determined.

Excluded from the coolants according to the general formula (VIa) are in particular those compounds in which in the general formula (VIa) R1 and R2 stand for phenyl, Y stands for branched alkyl, in particular Y stands for methylene which is substituted with —CH3 or with —CH2CO2C2H5, and Z in the general formula (VIa) stands for —NH-phenyl, —N(CH₃)-phenyl, —OH, —OC2H5 or —OC(CH3)3.

The physiological coolants according to the general formulae (I) to (VIII) and (Va) to (VIII) are present either in neutral, i.e., uncharged form, or in the form of their salts, e.g., as acid addition salts, with inorganic or organic acids.

The term “salt” in the context of the present invention refers to a salt of a compound that has the desired effect or pharmacological activity of the parent compound. Such salts include:
(1) Acid addition salts formed with inorganic acids or formed with organic acids, preferably mono- or polyvalent carboxylic acids; or
(2) Salts formed when an acidic proton present in the starting compound is replaced by a metal ion, e.g., an alkali metal ion, an alkaline earth ion or an aluminum ion; or coordinated with an organic base.

Among the salts, acid addition salts are again particularly preferred since the physiological coolant according to the general formulae (I) to (VIII) comprises a protonatable N atom.

The inorganic acids which form acid addition salts with the physiological coolants of the present invention are preferably selected from the group consisting of hydrochloric acid, hydrobromic acid, sulfuric acid, nitric acid, phosphoric acid and the like. Among the salts most preferred are the hydrochlorides or sulfates. Particularly preferred is the hydrochloride salt or the sulfate salt.

Even more preferred are acid addition salts with organic mono- or polycarboxylic acids. Further preferred are acid addition salts with organic mono- or polycarboxylic acids, wherein the carboxylic acid is selected from saturated or mono- or polyunsaturated C1 to C30 monocarboxylic acids, saturated or mono- or polyunsaturated C3 to 10 di- or tricarboxylic acids. The carboxylic acid can be mono- or polysubstituted with hydroxy groups, preferably α-hydroxycarboxylic acids in which the hydroxy group is located on the carbon atom adjacent to the carboxy group. Many representatives occur naturally as so-called fruit acids. Preferred α-hydroxycarboxylic acids are malic acid, citric acid, 2-hydroxy-4-methylmercaptobutyric acid, glycolic acid, isocitric acid, mandelic acid, lactic acid, tartronic acid or tartaric acid.

The organic acids which form acid addition salts with the physiological coolants according to the present invention are preferably selected from the group consisting of amino acids, acetic acid, trifluoroacetic acid, propionic acid, hexanoic acid, cyclopentanepropionic acid, glycolic acid, pyruvic acid, lactic acid, malonic acid, oxalic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphor sulfonic acid, 4-methylbicyclo[2.2.2]-oct-2-ene-1-carboxylic acid, glucoheptonic acid, 3-phenylpropionic acid, trimethyl acetic acid, tert.butylacetic acid, laurylsulfuric acid, gluconic acid, glutamic acid, hydroxynaphthoic acid, salicylic acid, stearic acid, muconic acid, 4-hydroxybutanoic acid, and the like.

Among the organic acids which form acid addition salts with the physiological coolants of the present invention, the most preferred are acetic acid, lactic acid, malonic acid, succinic acid, malic acid, citric acid or tartaric acid.

The metal ions for salt formation, which replace an acidic proton present in the starting compound, are selected from the group consisting of alkali metal ions, preferably Na+ or K+, alkaline earth metal ions, preferably Ca++, Mg++, and aluminum+++.

The coordinating organic base for salt formation is selected from the group consisting of ethanolamine, diethanolamine, triethanolamine, N-methylglucamine and the like.

In the following description and in the patent claims, the terms “physiological coolant” or “compound” include both the neutral, uncharged form of the coolant/compound and equally the salt form of the coolant/compound.

Due to their better solubility, the salts of the physiological coolants according to the present invention are particularly preferred. The better water solubility also results in better availability of the coolants or compounds during their use.

Surprisingly, it was found that the compounds or coolants according to the invention or their salts have the common property of achieving a particularly long-lasting and intensive cooling effect on the skin or mucous membrane in vivo even at low dosages in the low ppm range of about 5 ppm. This means that in the final preparation, a lower dosage of the coolant according to the invention or its salt is required in order to achieve an intensive cooling effect. Thus, the compounds described herein are particularly efficient cooling substances. This was not foreseeable for the TRPM8 modulators mentioned in this application.

The coolants according to the invention are further characterized by the fact that their cooling effect starts quickly; other coolants according to the invention, on the other hand, have a build-up cooling effect, i.e., a cooling effect that increases over time and produces a longer and more intensive cooling effect.

The coolants according to the invention are also colorless and non-discoloring, which is particularly advantageous for their storage and/or use in the end product. Consequently, the compounds described herein are characterized as particularly suitable additives in various preparations. Furthermore, the compounds according to the invention described herein are largely tasteless and odorless, so that they are also excellently suited for incorporation into neutral and/or flavored preparations without creating a taste impression that is perceived as negative, for example as bitter, or adversely affecting the intended taste or odor impression.

The salts of the coolants according to the invention show better solubility in vitro than their neutral, uncharged equivalents, which is particularly advantageous when they are used in the oral care sector.

Up to now, there has been no evidence in the prior art that the compounds to be used according to the invention or their salts in particular can produce a cooling effect at all, and certainly not a particularly long-lasting cooling effect.

Equally surprising was the fact that the coolants or their salts according to the invention are able to mask the known taste disadvantages of flavors, especially of sweeteners such as steviosides. In particular, the pungent, bitter and metallic aftertaste is effectively masked even when small amounts are added.

Thus, the compounds described herein are suitable as particularly efficient cooling substances, which can be incorporated particularly well into a variety of formulations. Due to their improved solubility, the salts, and even more preferably the acid addition salts, of the compounds according to the invention are particularly advantageous for use in the oral care sector.

Particularly preferred are the physiological coolants of the general formulae (I), (II), (V) or (VI), selected from the group consisting of the compounds shown in Table 1:

TABLE 1
Structures according to the invention with relative TRPM8 activation in %

| Com- | TRPM8 | | |
|---|---|---|---|
| pound | activation | Structure | IUPAC name |
| B-01 | 262 | 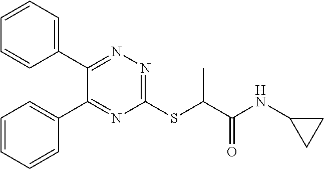 [SMILES: CC(Sc1nnc(-c2ccccc2)c(-c2ccccc2)n1)C(=O)NC1CC1] | N-Cyclopropyl-2-[(5,6- diphenyl-1,2,4-triazin-3- yl)sulfanyl]propanamide |
| B-02 | 227 | 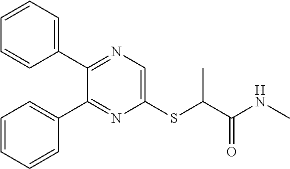 [SMILES: CNC(=O)C(C)Sc1cnc(-c2ccccc2)c(-c2ccccc2)n1] | 2-(5,6-diphenylpyrazin-2- yl)sulfanyl-N-methyl- propanamide |
| B-03 | 221 | 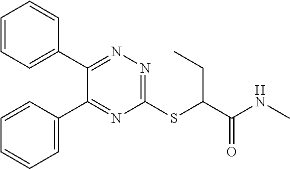 [SMILES: CCC(Sc1nnc(-c2ccccc2)c(-c2ccccc2)n1)C(=O)NC] | 2-[(5,6-diphenyl-1,2,4-triazin- 3-yl)sulfanyl]-N-methyl- butanamide |
| B-04 | 210 | 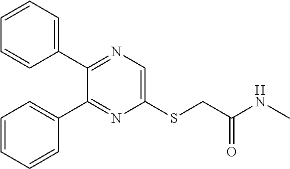 [SMILES: CNC(=O)CSc1cnc(-c2ccccc2)c(-c2ccccc2)n1] | 2-(5,6-diphenylpyrazin-2- yl)sulfanyl-N-methyl- acetamide |
| B-05 | 205 | 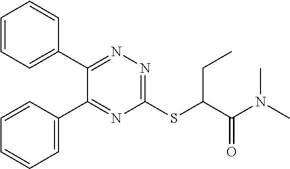 [SMILES: CCC(Sc1nnc(-c2ccccc2)c(-c2ccccc2)n1)C(=O)N(C)C] | 2-[(5,6-diphenyl-1,2,4-triazin- 3-yl)sulfanyl]-N,N-dimethyl- butanamide |
| B-06 | 205 | 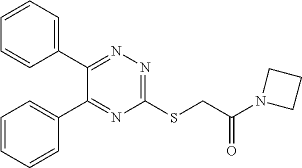 [SMILES: O=C(CSc1nnc(-c2ccccc2)c(-c2ccccc2)n1)N1CCC1] | 1-(azetidin-1-yl)-2-[(5,6- diphenyl-1,2,4-triazin-3- yl)sulfanyl]ethanone |
| B-07 | 203 | 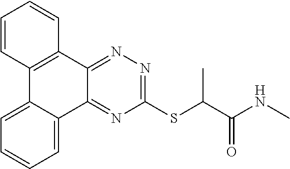 [SMILES: CNC(=O)C(C)Sc1nnc2c3ccccc3c3ccccc3c2n1] | N-methyl-2-phenanthro[9,10- e][1,2,4]triazine-3-ylsulfanyl- propanamide |
| B-11 | 143 | 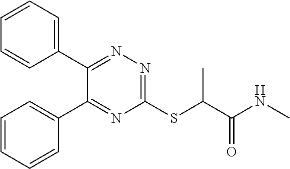 [SMILES: CNC(=O)C(C)Sc1nnc(-c2ccccc2)c(-c2ccccc2)n1] | 2-[(5,6-diphenyl-1,2,4-triazin- 3-yl)sulfanyl]-N-methyl- propanamide |
| B-14 | 122 | 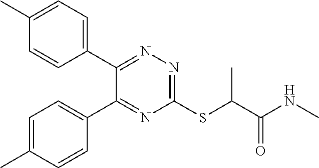 | 2-[[5,6-bis(p-tolyl)-1,2,4- triazin-3-yl]sulfanyl]-N- methyl-propanamide |
| B-15 | 109 | 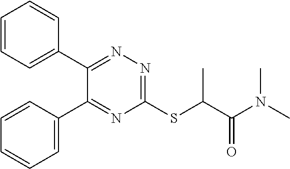 [SMILES: CC(Sc1nnc(-c2ccccc2)c(-c2ccccc2)n1)C(=O)N(C)C] | 2-[(5,6-diphenyl-1,2,4-triazin- 3-yl)sulfanyl]-N,N-dimethyl- propanamide |
| B-17 | 102 | 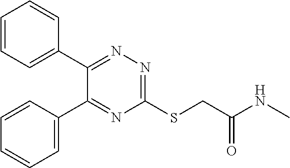 [SMILES: CNC(=O)CSc1nnc(-c2ccccc2)c(-c2ccccc2)n1] | 2-[(5,6-diphenyl-1,2,4-triazin- 3-yl)sulfanyl]-N-methyl- acetamide |
| B-18 | 101 | 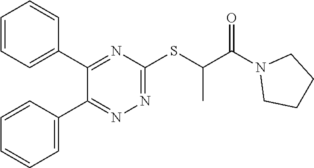 [SMILES: CC(Sc1nnc(-c2ccccc2)c(-c2ccccc2)n1)C(=O)N1CCCC1] | 2-[(5,6-diphenyl-1,2,4-triazin- 3-yl)sulfanyl]-1-pyrrolidin-1- yl-propan-1-one |
| B-19 | 101 | 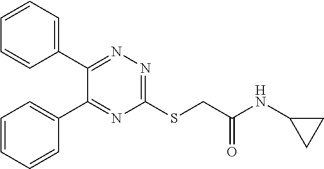 [SMILES: O=C(CSc1nnc(-c2ccccc2)c(-c2ccccc2)n1)NC1CC1] | N-cyclopropyl-2-[(5,6- diphenyl-1,2,4-triazin-3- yl)sulfanyl]acetamide |
| B-21 | 90 | 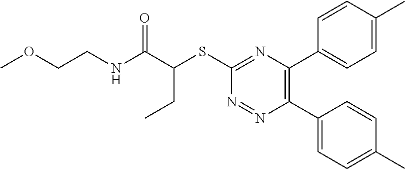 | 2-[[5,6-bis(p-tolyl)-1,2,4- triazin-3-yl]sulfanyl]-N-(2- methoxyethyl)butanamide |
| B-22 | 87 | 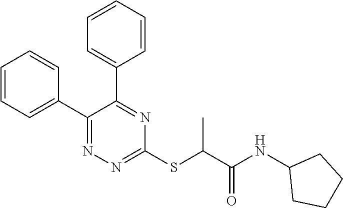 [SMILES: CC(Sc1nnc(-c2ccccc2)c(-c2ccccc2)n1)C(=O)NC1CCCC1] | N-cyclopentyl-2-[(5,6- diphenyl-1,2,4-triazin-3- yl)sulfanyl]propanamide |
| B-23 | 73 | 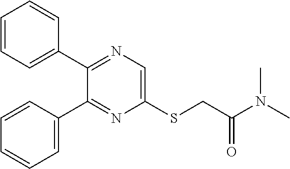 [SMILES: CN(C)C(=O)CSc1cnc(-c2ccccc2)c(-c2ccccc2)n1] | 2-(5,6-diphenylpyrazin-2- yl)sulfanyl-N,N-dimethyl- acetamide |
| B-24 | 68 | 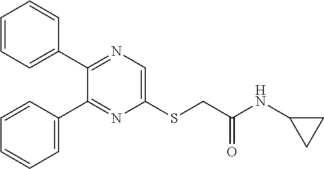 [SMILES: O=C(CSc1cnc(-c2ccccc2)c(-c2ccccc2)n1)NC1CC1] | N-cyclopropyl-2-(5,6- diphenylpyrazin-2-yl)sulfanyl- acetamide |
| B-25 | 57 | 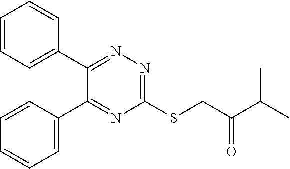 [SMILES: CC(C)C(=O)CSc1nnc(-c2ccccc2)c(-c2ccccc2)n1] | 1-[(5,6-diphenyl-1,2,4-triazin- 3-yl)sulfanyl]-3-methyl-butan- 2-one |
| B-26 | 56 | 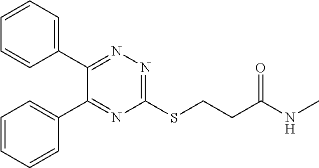 [SMILES: CNC(=O)CCSc1nnc(-c2ccccc2)c(-c2ccccc2)n1] | 3-[(5,6-diphenyl-1,2,4-triazin- 3-yl)sulfanyl]-N-methyl- propanamide |
| B-27 | 56 | 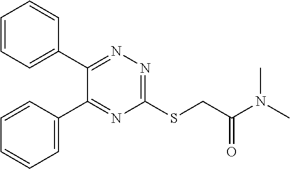 [SMILES: CN(C)C(=O)CSc1nnc(-c2ccccc2)c(-c2ccccc2)n1] | 2-[(5,6-diphenyl-1,2,4-triazin- 3-yl)sulfanyl]-N,N-dimethyl- acetamide |
| B-28 | 53 | 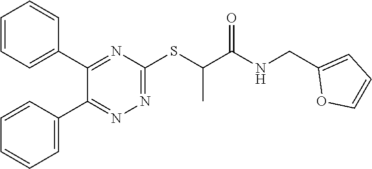 [SMILES: CC(Sc1nnc(-c2ccccc2)c(-c2ccccc2)n1)C(=O)NCc1ccco1] | 2-[(5,6-diphenyl-1,2,4-triazin- 3-yl)sulfanyl]-N-(2- furylmethyl)propanamide |
| B-29 | 42 | 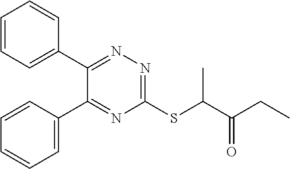 [SMILES: CCC(=O)C(C)Sc1nnc(-c2ccccc2)c(-c2ccccc2)n1] | 2-[(5,6-diphenyl-1,2,4-triazin- 3-yl)sulfanyl]pentan-3-one |
| B-30 | 41 | 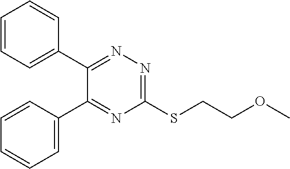 [SMILES: COCCSc1nnc(-c2ccccc2)c(-c2ccccc2)n1] | 3-(2-methoxyethylsulfanyl)- 5,6-diphenyl-1,2,4-triazine |
| B-31 | 40 | 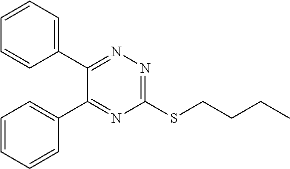 [SMILES: CCCCSc1nnc(-c2ccccc2)c(-c2ccccc2)n1] | 3-butylsulfanyl-5,6-diphenyl- 1,2,4-triazine |
| B-32 | 37 | 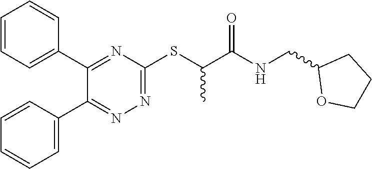 [SMILES: CC(Sc1nnc(-c2ccccc2)c(-c2ccccc2)n1)C(=O)NCC1CCCO1] | 2-[(5,6-diphenyl-1,2,4-triazin- 3-yl)sulfanyl]-N- (tetrahydrofuran-2- ylmethyl)propanamide |
| B-33 | 25 | 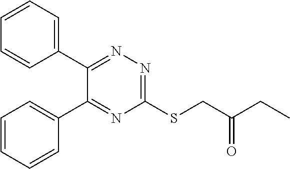 [SMILES: CCC(=O)CSc1nnc(-c2ccccc2)c(-c2ccccc2)n1] | 1-[(5,6-diphenyl-1,2,4-triazin- 3-yl)sulfanyl]butan-2-one |
| B-34 | 25 | 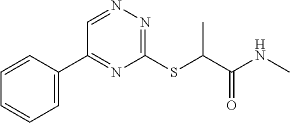 [SMILES: CNC(=O)C(C)Sc1nncc(-c2ccccc2)n1] | N-methyl-2-[(5-phenyl-1,2,4- triazin-3- yl)sulfanyl]propanamide |
| B-36 | 20 | 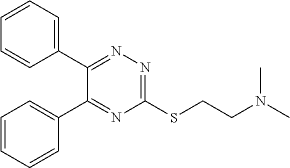 [SMILES: CN(C)CCSc1nnc(-c2ccccc2)c(-c2ccccc2)n1] | 2-[(5,6-diphenyl-1,2,4-triazin- 3-yl)sulfanyl]-N,N-dimethyl- ethanamine |
| B-37 | 13 | 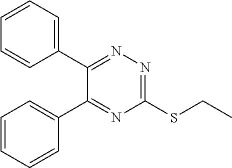 [SMILES: CCSc1nnc(-c2ccccc2)c(-c2ccccc2)n1] | 3-ethylsulfanyl-5,6-diphenyl- 1,2,4-triazine |
| B-38 | 11 | 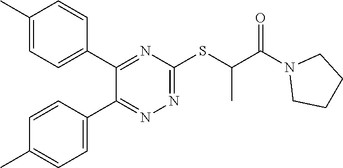 [SMILES: Cc1ccc(-c2nnc(SC(C)C(=O)N3CCCC3)nc2-c2ccc(C)cc2)cc1] | 2-[[5,6-bis(p-tolyl)-1,2,4- triazin-3-yl]sulfanyl]-1- pyrrolidin-1-yl-propan-1-one |
| B-39 | 11 | 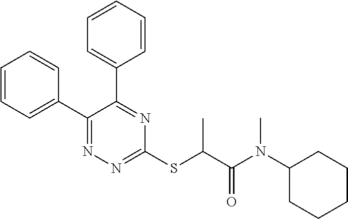 [SMILES: CC(Sc1nnc(-c2ccccc2)c(-c2ccccc2)n1)C(=O)N(C)C1CCCCC1] | N-cyclohexyl-2-[(5,6- diphenyl-1,2,4-triazin-3- yl)sulfanyl]-N-methyl- propanamide |
| B-40 | 9 | 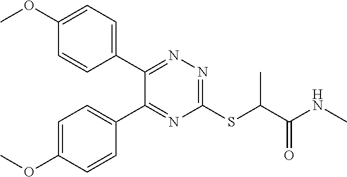 | 2-[[5,6-bis(4-methoxyphenyl)- 1,2,4-triazin-3-yl]sulfanyl]-N- methyl-propanamide |
| B-41 | 8 | 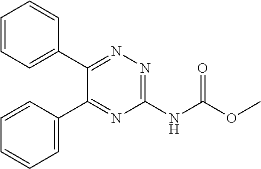 [SMILES: COC(=O)Nc1nnc(-c2ccccc2)c(-c2ccccc2)n1] | methyl N-(5,6-diphenyl-1,2,4- triazin-3-yl)carbamate |
| B-42 | 8 | 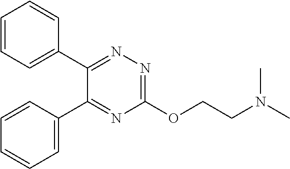 [SMILES: CN(C)CCOc1nnc(-c2ccccc2)c(-c2ccccc2)n1] | 2-[(5,6-diphenyl-1,2,4-triazin- 3-yl)oxy]-N,N-dimethyl- ethanamine |
| B-43 | 6 | 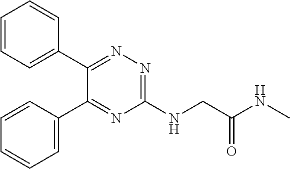 [SMILES: CNC(=O)CNc1nnc(-c2ccccc2)c(-c2ccccc2)n1] | 2-[(5,6-diphenyl-1,2,4-triazin- 3-yl)amino]-N-methyl- acetamide |
| B-44 | 5 | 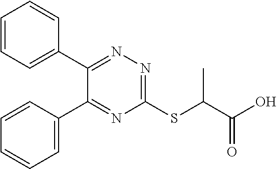 [SMILES: CC(Sc1nnc(-c2ccccc2)c(-c2ccccc2)n1)C(=O)O] | 2-[(5,6-diphenyl-1,2,4-triazin- 3-yl)sulfanyl]propanoic acid |
| B-45 | 5 | 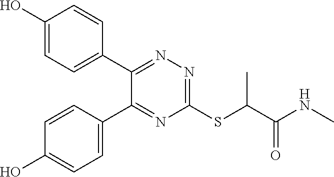 [SMILES: CNC(=O)C(C)Sc1nnc(-c2ccc(O)cc2)c(-c2ccc(O)cc2)n1] | 2-[[5,6-bis(4-hydroxyphenyl)- 1,2,4-triazin-3-yl]sulfanyl]-N- methyl-propanamide |
| B-46 | 5 | 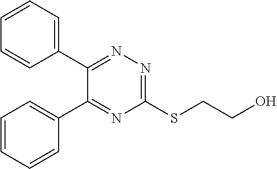 [SMILES: OCCSc1nnc(-c2ccccc2)c(-c2ccccc2)n1] | 2-[(5,6-diphenyl-1,2,4-triazin- 3-yl)sulfanyl]ethanol |
| B-47 | 4 | 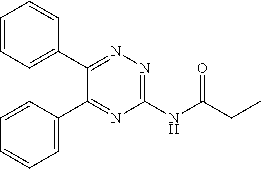 [SMILES: CCC(=O)Nc1nnc(-c2ccccc2)c(-c2ccccc2)n1] | N-(5,6-diphenyl-1,2,4-triazin- 3-yl)propanamide |
| B-48 | 4 | 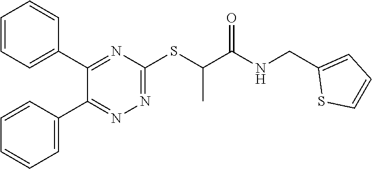 [SMILES: CC(Sc1nnc(-c2ccccc2)c(-c2ccccc2)n1)C(=O)NCc1cccs1] | 2-[(5,6-diphenyl-1,2,4-triazin- 3-yl)sulfanyl]-N-(2- thienylmethyl)propanamide |
| B-49 | 3 | 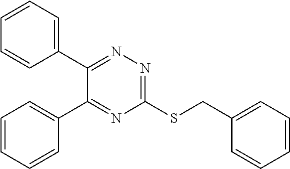 [SMILES: c1ccc(CSc2nnc(-c3ccccc3)c(-c3ccccc3)n2)cc1] | 3-benzylsulfanyl-5,6- diphenyl-1,2,4-triazine |
| B-50 | 3 | 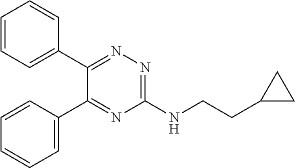 [SMILES: c1ccc(-c2nnc(NCCC3CC3)nc2-c2ccccc2)cc1] | N-(2-cyclopropylethyl)-5,6- diphenyl-1,2,4-triazin-3- amine |
| B-51 | 3 | 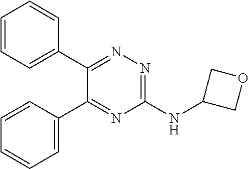 [SMILES: c1ccc(-c2nnc(NC3COC3)nc2-c2ccccc2)cc1] | N-(oxetan-3-yl)-5,6-diphenyl- 1,2,4-triazin-3-amine |
| B-52 | 3 | 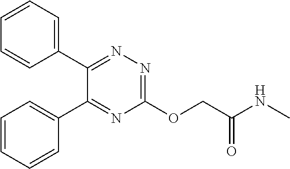 [SMILES: CNC(=O)COc1nnc(-c2ccccc2)c(-c2ccccc2)n1] | 2-[(5,6-diphenyl-1,2,4-triazin- 3-yl)oxy]-N-methyl-acetamide |
| B-53 | 2 | 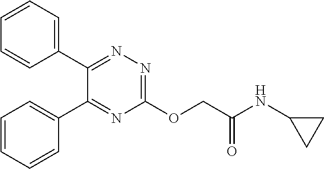 [SMILES: O=C(COc1nnc(-c2ccccc2)c(-c2ccccc2)n1)NC1CC1] | N-cyclopropyl-2-[(5,6- diphenyl-1,2,4-triazin-3- yl)oxy]acetamide |
| B-54 | 2 | 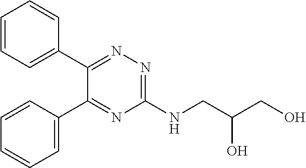 [SMILES: OCC(O)CNc1nnc(-c2ccccc2)c(-c2ccccc2)n1] | 3-[(5,6-diphenyl-1,2,4-triazin- 3-yl)amino]propane-1,2-diol |
| B-55 | 2 | 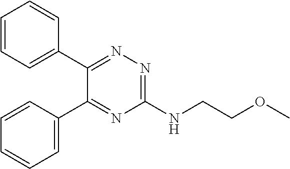 [SMILES: COCCNc1nnc(-c2ccccc2)c(-c2ccccc2)n1] | N-(2-methoxyethyl)-5,6- diphenyl-1,2,4-triazin-3- amine |
| B-56 | 2 | 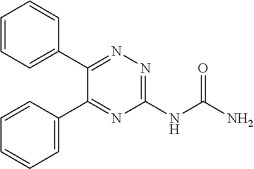 [SMILES: NC(=O)Nc1nnc(-c2ccccc2)c(-c2ccccc2)n1] | (5,6-diphenyl-1,2,4-triazin-3- yl)urea |
| B-57 | 2 | 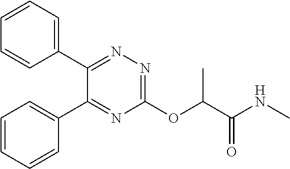 [SMILES: CNC(=O)C(C)Oc1nnc(-c2ccccc2)c(-c2ccccc2)n1] | 2-[(5,6-diphenyl-1,2,4-triazin- 3-yl)oxy]-N-methyl- propanamide |
| B-58 | 1 | 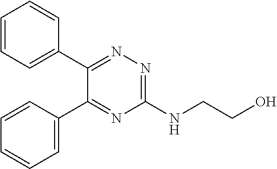 [SMILES: OCCNc1nnc(-c2ccccc2)c(-c2ccccc2)n1] | 2-[(5,6-diphenyl-1,2,4-triazin- 3-yl)amino]ethanol |
| B-59 | 1 | 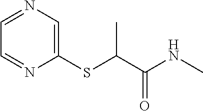 | N-methyl-2-pyrazine-2- ylsulfanyl-propanamide |
| B-60 | 1 | 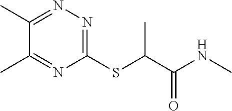 | 2-[(5,6-dimethyl-1,2,4-triazin- 3-yl)sulfanyl]-N-methyl- propanamide |
| B-61 | 1 | 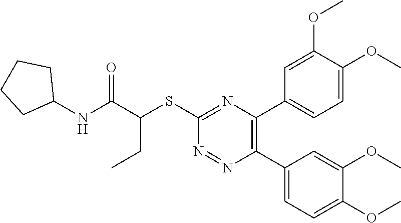 [SMILES: CCC(Sc1nnc(-c2ccc(OC)c(OC)c2)c(-c2ccc(OC)c(OC)c2)n1)C(=O)NC1CCCC1] | 2-[[5,6-bis(3,4- dimethoxyphenyl)-1,2,4- triazin-3-yl]sulfanyl]-N- cyclopentyl-butanamide |
| B-62 | 0 | 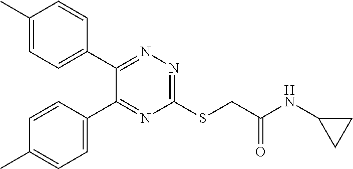 [SMILES: Cc1ccc(-c2nnc(SCC(=O)NC3CC3)nc2-c2ccc(C)cc2)cc1] | 2-[[5,6-bis(p-tolyl)-1,2,4- triazin-3-yl]sulfanyl]-N- cyclopropyl-acetamide |
| B-63 | −2 | 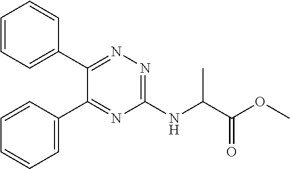 [SMILES: COC(=O)C(C)Nc1nnc(-c2ccccc2)c(-c2ccccc2)n1] | methyl 2-[(5,6-diphenyl-1,2,4- triazin-3-yl)amino]propanoate |
| B-64 | −3 | 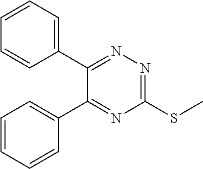 [SMILES: CSc1nnc(-c2ccccc2)c(-c2ccccc2)n1] | 3-methylsulfanyl-5,6- diphenyl-1,2,4-triazine |
| B-65 | | 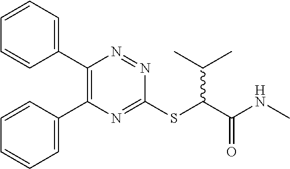 [SMILES: CNC(=O)C(Sc1nnc(-c2ccccc2)c(-c2ccccc2)n1)C(C)C] | 2-[(5,6-diphenyl-1,2,4-triazin- 3-yl)sulfanyl]-N,3-dimethyl- butanamide |
| B-66 | | 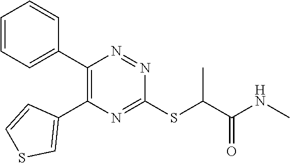 [SMILES: CNC(=O)C(C)Sc1nnc(-c2ccccc2)c(-c2ccsc2)n1] | N-methyl-2-[[6-phenyl-5-(3- thienyl)-1,2,4-triazin-3- yl]sulfanyl]propanamide |
| B-67 | | 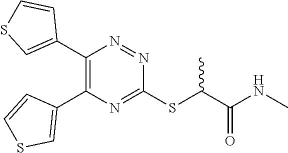 [SMILES: CNC(=O)C(C)Sc1nnc(-c2ccsc2)c(-c2ccsc2)n1] | 2-[[5,6-bis(3-thienyl)-1,2,4- triazin-3-yl]sulfanyl]-N- methyl-propanamide |
| B-68 | | 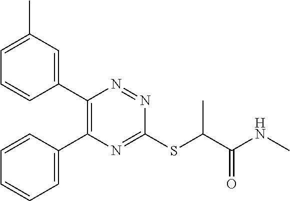 [SMILES: CNC(=O)C(C)Sc1nnc(-c2cccc(C)c2)c(-c2ccccc2)n1] | N-methyl-2-[[6-(m-tolyl)-5- phenyl-1,2,4-triazin-3- yl]sulfanyl]propanamide |
| B-69 | | 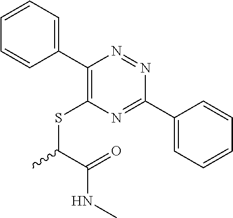 [SMILES: CNC(=O)C(C)Sc1nc(-c2ccccc2)nnc1-c1ccccc1] | 2-[(3,6-diphenyl-1,2,4-triazin- 5-yl)sulfanyl]-N-methyl- propanamide |
| B-70 | | 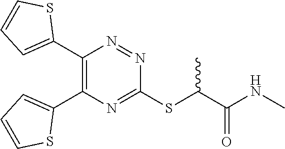 [SMILES: CNC(=O)C(C)Sc1nnc(-c2cccs2)c(-c2cccs2)n1] | 2-[[5,6-bis(2-thienyl)-1,2,4- triazin-3-yl]sulfanyl]-N- methyl-propanamide |
| B-71 | | 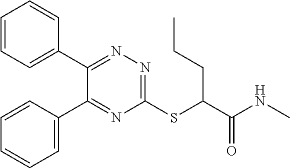 [SMILES: CCCC(Sc1nnc(-c2ccccc2)c(-c2ccccc2)n1)C(=O)NC] | 2-[(5,6-diphenyl-1,2,4-triazin- 3-yl)sulfanyl]-N-methyl- pentanamide |
| B-72 | | 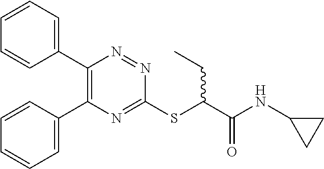 [SMILES: CCC(Sc1nnc(-c2ccccc2)c(-c2ccccc2)n1)C(=O)NC1CC1] | N-cyclopropyl-2-[(5,6- diphenyl-1,2,4-triazin-3- yl)sulfanyl]butanamide |
| B-73 | | 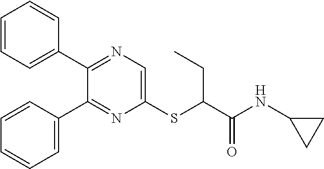 [SMILES: CCC(Sc1cnc(-c2ccccc2)c(-c2ccccc2)n1)C(=O)NC1CC1] | N-cyclopropyl-2-(5,6- diphenylpyrazin-2-yl)sulfanyl- butanamide |
| B-74 | | 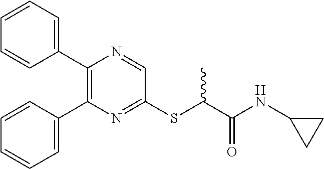 [SMILES: CC(Sc1cnc(-c2ccccc2)c(-c2ccccc2)n1)C(=O)NC1CC1] | N-cyclopropyl-2-(5,6- diphenylpyrazin-2-yl)sulfanyl- propanamide |
| B-75 | | 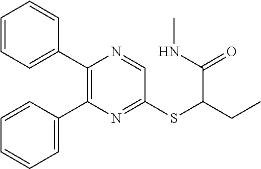 [SMILES: CCC(Sc1cnc(-c2ccccc2)c(-c2ccccc2)n1)C(=O)NC] | 2-[(5,6-Diphenylpyrazin-2- yl)sulfanyl]-N- methylbutanamide |
| B-76 | | 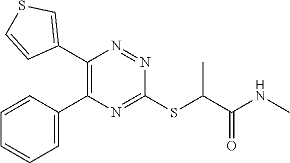 [SMILES: CNC(=O)C(C)Sc1nnc(-c2ccsc2)c(-c2ccccc2)n1] | N-methyl-2-[[5-phenyl-6-(3- thienyl)-1,2,4-triazin-3- yl]sulfanyl]propanamide |
| B-77 | | 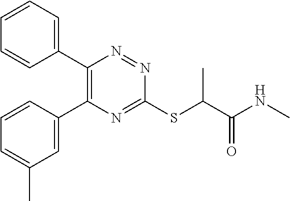 [SMILES: CNC(=O)C(C)Sc1nnc(-c2ccccc2)c(-c2cccc(C)c2)n1] | N-methyl-2-[[5-(m-tolyl)-6- phenyl-1,2,4-triazin-3- yl]sulfanyl]propanamide |

The coolants of the invention listed in Table 1 according to the general formulae (I), (II), (V) or (VI) are either present in neutral, uncharged form or are present in the form of their salts, for example as an acid addition salt, with inorganic or organic, mono- or polyvalent carboxylic acids, as described in detail above. In this respect, the above also applies here.

The coolants according to Table 1 can be present in stereoisomerically pure form or as mixtures of different stereoisomers and can therefore also be used in formulations in this way.

Surprisingly, it has been shown that the compounds according to Table 1 have particularly high TRPM8 activations and are therefore ideally suited as coolants.

The most preferred coolants, i.e. coolants with a particularly efficient and strong TRPM8 activation, i.e. efficient and intensive cooling effect with a low application quantity, are the compounds B-01, B-02, B-03, B-04, B-05, B-06, B-07, B-11, B14, B-15, B-17, B-18, B-19 and B-21 (TRPM8 activation 90%) and in particular the compounds B-01, B-02, B-03, B-04, B-05, B-06, B-07, B-11, B-14, B-15, B-17, B-18 and B-19 (TRPM8 activation 100%). Particularly preferred are the compounds B-01, B-02, B-03, B-04, B-05, B-06, B-07, which show an extraordinarily high TRPM8 activity (TRPM8 activation 150%).

Due to their outstanding relative TRPM8 activation, compounds B-01 (TRPM8 activation of 262%), B-02 (TRPM8 activation of 227%), B-03 (TRPM8 activation of 221%), B-04 (TRPM8 activation of 210%), B-05 (TRPM8 activation of 205%), B-06 (TRPM8 activation of 205%) and B-07 (TRPM8 activation of 203%) are the most preferred.

The compound B-01 (triazine derivative) is characterized in that in the general formula (V) R1 and R2 represent a phenyl group, Y represents a branched methylene group substituted with an ethyl group, and Z represents an —NH-cyclopropyl group.

The compound B-02 (pyrazine derivative) is characterized in that in the general formula (VI) R1 and R2 represent a phenyl group, Y represents a branched methylene group substituted with an ethyl group, and Z represents an —NH—CH₃-group.

The compound B-03 (triazine derivative) is characterized in that in the general formula (V) R1 and R2 represent a phenyl group, Y represents a branched methylene group substituted with an ethyl group, and Z represents an —NH—CH₃— group.

The compound B-04 (pyrazine derivative) is characterized in that in the general formula (VI) R1 and R2 represent a phenyl group, Y represents a methylene group, and Z represents an —NH—CH₃-group.

The compound B-05 (triazine derivative) is characterized in that in the general formula (V) R1 and R2 represent a phenyl group, Y represents a branched methylene group substituted with an ethyl group, and Z represents an —N(CH₃)₂-group.

The compound B-06 (triazine derivative) is characterized in that in the general formula (V) R1 and R2 represent a phenyl group, Y represents a methylene group and Z represents an azetidine group.

The compound B-07 (triazine derivative) is characterized in that in the general formula (V) R1 and R2 represent a phenyl group which, together with the C atoms of the triazine ring to which they are attached, form a fused ring system, i.e. a 1,2,4-triazatriphenylene, Y represents a branched methylene group substituted with a methyl group, and Z represents an NH—CH₃-group.

Even more preferred are the physiological coolants of the general formulae (Va) or (VIa) selected from the group consisting of the compounds shown in Table A:

TABLE A
Structures according to the invention with relative TRPM8 activation in %

| Com- | TRPM8 | | |
|---|---|---|---|
| pound | activation | Structure | IUPAC name |
| B-01 | 262 | 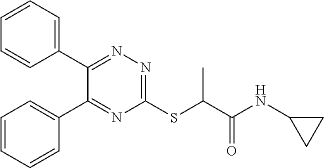 [SMILES: CC(Sc1nnc(-c2ccccc2)c(-c2ccccc2)n1)C(=O)NC1CC1] | N-Cyclopropyl-2-[(5,6- diphenyl-1,2,4-triazin-3- yl)sulfanyl]propanamide |
| B-02 | 227 | 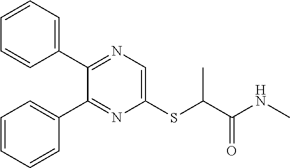 [SMILES: CNC(=O)C(C)Sc1cnc(-c2ccccc2)c(-c2ccccc2)n1] | 2-(5,6-diphenylpyrazin-2- yl)sulfanyl-N-methyl- propanamide |
| B-03 | 221 | 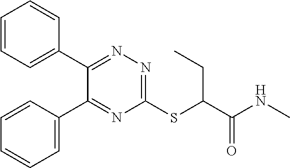 [SMILES: CCC(Sc1nnc(-c2ccccc2)c(-c2ccccc2)n1)C(=O)NC] | 2-[(5,6-diphenyl-1,2,4-triazin- 3-yl)sulfanyl]-N-methyl- butanamide |
| B-05 | 205 | 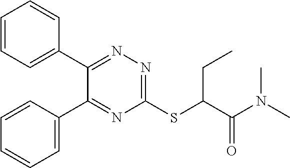 [SMILES: CCC(Sc1nnc(-c2ccccc2)c(-c2ccccc2)n1)C(=O)N(C)C] | 2-[(5,6-diphenyl-1,2,4-triazin- 3-yl)sulfanyl]-N,N-dimethyl- butanamide |
| B-07 | 203 | 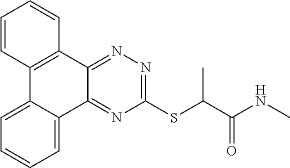 [SMILES: CNC(=O)C(C)Sc1nnc2c3ccccc3c3ccccc3c2n1] | N-methyl-2-phenanthro[9,10- e][1,2,4]triazine-3-ylsulfanyl- propanamide |
| B-11 | 143 | 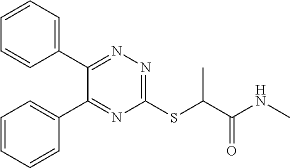 [SMILES: CNC(=O)C(C)Sc1nnc(-c2ccccc2)c(-c2ccccc2)n1] | 2-[(5,6-diphenyl-1,2,4-triazin- 3-yl)sulfanyl]-N-methyl- propanamide |
| B-14 | 122 | 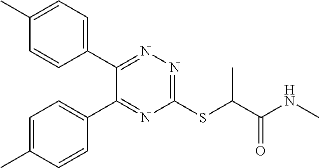 | 2-[[5,6-bis(p-tolyl)-1,2,4- triazin-3-yl]sulfanyl]-N- methyl-propanamide |
| B-15 | 109 | 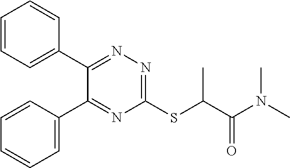 [SMILES: CC(Sc1nnc(-c2ccccc2)c(-c2ccccc2)n1)C(=O)N(C)C] | 2-[(5,6-diphenyl-1,2,4-triazin- 3-yl)sulfanyl]-N,N-dimethyl- propanamide |
| B-18 | 101 | 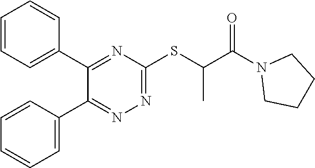 [SMILES: CC(Sc1nnc(-c2ccccc2)c(-c2ccccc2)n1)C(=O)N1CCCC1] | 2-[(5,6-diphenyl-1,2,4-triazin- 3-yl)sulfanyl]-1-pyrrolidin-1- yl-propan-1-one |
| B-21 | | 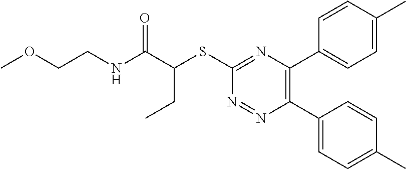 | 2-[[5,6-bis(p-tolyl)-1,2,4- triazin-3-yl]sulfanyl]-N-(2- methoxyethyl)butanamide |
| B-22 | 87 | 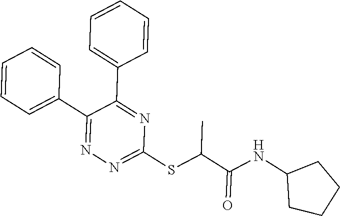 [SMILES: CC(Sc1nnc(-c2ccccc2)c(-c2ccccc2)n1)C(=O)NC1CCCC1] | N-cyclopentyl-2-[(5,6- diphenyl-1,2,4-triazin-3- yl)sulfanyl]propanamide |
| B-28 | 53 | 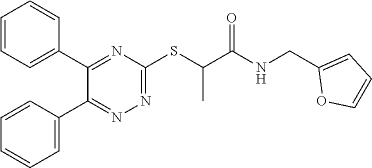 [SMILES: CC(Sc1nnc(-c2ccccc2)c(-c2ccccc2)n1)C(=O)NCc1ccco1] | 2-[(5,6-diphenyl-1,2,4-triazin- 3-yl)sulfanyl]-N-(2- furylmethyl)propanamide |
| B-29 | 42 | 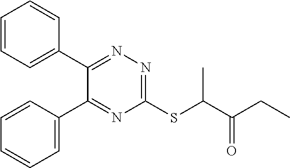 [SMILES: CCC(=O)C(C)Sc1nnc(-c2ccccc2)c(-c2ccccc2)n1] | 2-[(5,6-diphenyl-1,2,4-triazin- 3-yl)sulfanyl]pentan-3-one |
| B-32 | 37 | 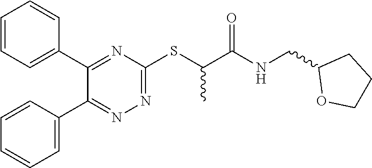 [SMILES: CC(Sc1nnc(-c2ccccc2)c(-c2ccccc2)n1)C(=O)NCC1CCCO1] | 2-[(5,6-diphenyl-1,2,4-triazin- 3-yl)sulfanyl]-N- (tetrahydrofuran-2- ylmethyl)propanamide |
| B-34 | 25 | 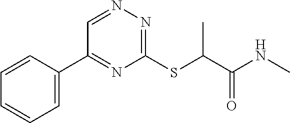 [SMILES: CNC(=O)C(C)Sc1nncc(-c2ccccc2)n1] | N-methyl-2-[(5-phenyl-1,2,4- triazin-3- yl)sulfanyl]propanamide |
| B-38 | 11 | 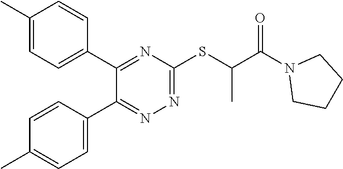 [SMILES: Cc1ccc(-c2nnc(SC(C)C(=O)N3CCCC3)nc2-c2ccc(C)cc2)cc1] | 2-[[5,6-bis(p-tolyl)-1,2,4- triazin-3-yl]sulfanyl]-1- pyrrolidin-1-yl-propan-1-one |
| B-39 | 11 | 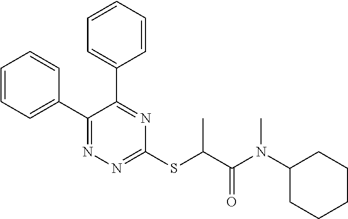 [SMILES: CC(Sc1nnc(-c2ccccc2)c(-c2ccccc2)n1)C(=O)N(C)C1CCCCC1] | N-cyclohexyl-2-[(5,6- diphenyl-1,2,4-triazin-3- yl)sulfanyl]-N-methyl- propanamide |
| B-40 | 9 | 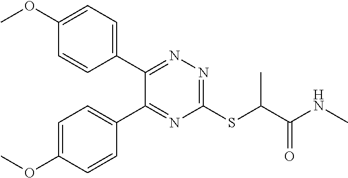 | 2-[[5,6-bis(4-methoxyphenyl)- 1,2,4-triazin-3-yl]sulfanyl]-N- methyl-propanamide |
| B-44 | 5 | 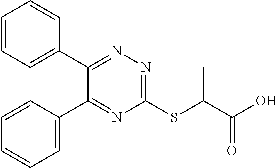 [SMILES: CC(Sc1nnc(-c2ccccc2)c(-c2ccccc2)n1)C(=O)O] | 2-[(5,6-diphenyl-1,2,4-triazin- 3-yl)sulfanyl]propanoic acid |
| B-45 | 5 | 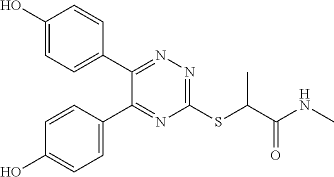 [SMILES: CNC(=O)C(C)Sc1nnc(-c2ccc(O)cc2)c(-c2ccc(O)cc2)n1] | 2-[[5,6-bis(4-hydroxyphenyl)- 1,2,4-triazin-3-yl]sulfanyl]-N- methyl-propanamide |
| B-48 | 4 | 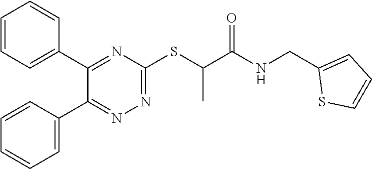 [SMILES: CC(Sc1nnc(-c2ccccc2)c(-c2ccccc2)n1)C(=O)NCc1cccs1] | 2-[(5,6-diphenyl-1,2,4-triazin- 3-yl)sulfanyl]-N-(2- thienylmethyl)propanamide |
| B-59 | 1 | 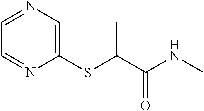 | N-methyl-2-pyrazine-2- ylsulfanyl-propanamide |
| B-60 | 1 | 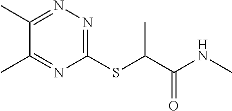 | 2-[(5,6-dimethyl-1,2,4-triazin- 3-yl)sulfanyl]-N-methyl- propanamide |
| B-61 | 1 | 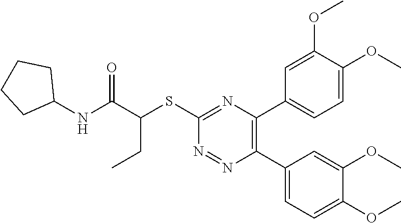 [SMILES: CCC(Sc1nnc(-c2ccc(OC)c(OC)c2)c(-c2ccc(OC)c(OC)c2)n1)C(=O)NC1CCCC1] | 2-[[5,6-bis(3,4- dimethoxyphenyl)-1,2,4- triazin-3-yl]sulfanyl]-N- cyclopentyl-butanamide |
| B-65 | | 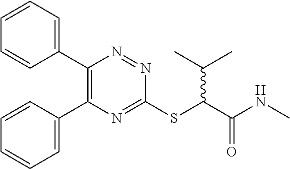 [SMILES: CNC(=O)C(Sc1nnc(-c2ccccc2)c(-c2ccccc2)n1)C(C)C] | 2-[(5,6-diphenyl-1,2,4-triazin- 3-yl)sulfanyl]-N,3-dimethyl- butanamide |
| B-66 | | 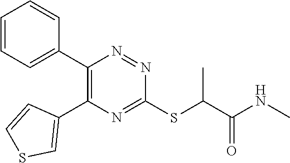 [SMILES: CNC(=O)C(C)Sc1nnc(-c2ccccc2)c(-c2ccsc2)n1] | N-methyl-2-[[6-phenyl-5-(3- thienyl)-1,2,4-triazin-3- yl]sulfanyl]propanamide |
| B-67 | | 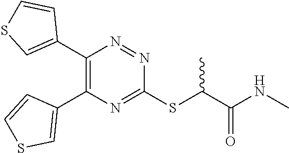 [SMILES: CNC(=O)C(C)Sc1nnc(-c2ccsc2)c(-c2ccsc2)n1] | 2-[[5,6-bis(3-thienyl)-1,2,4- triazin-3-yl]sulfanyl]-N- methyl-propanamide |
| B-68 | | 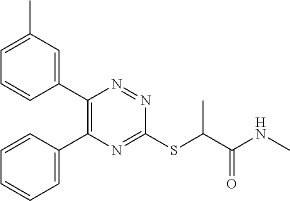 [SMILES: CNC(=O)C(C)Sc1nnc(-c2cccc(C)c2)c(-c2ccccc2)n1] | N-methyl-2-[[6-(m-tolyl)-5- phenyl-1,2,4-triazin-3- yl]sulfanyl]propanamide |
| B-69 | | 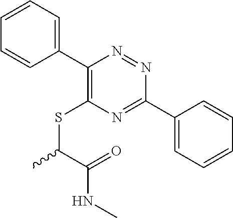 [SMILES: CNC(=O)C(C)Sc1nc(-c2ccccc2)nnc1-c1ccccc1] | 2-[(3,6-diphenyl-1,2,4-triazin- 5-yl)sulfanyl]-N-methyl- propanamide |
| B-70 | | 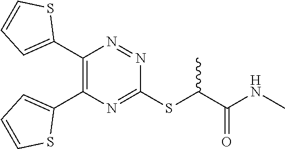 [SMILES: CNC(=O)C(C)Sc1nnc(-c2cccs2)c(-c2cccs2)n1] | 2-[[5,6-bis(2-thienyl)-1,2,4- triazin-3-yl]sulfanyl]-N- methyl-propanamide |
| B-71 | | 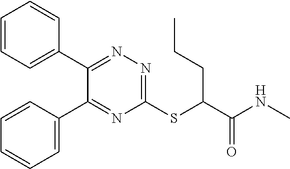 [SMILES: CCCC(Sc1nnc(-c2ccccc2)c(-c2ccccc2)n1)C(=O)NC] | 2-[(5,6-diphenyl-1,2,4-triazin- 3-yl)sulfanyl]-N-methyl- pentanamide |
| B-72 | | 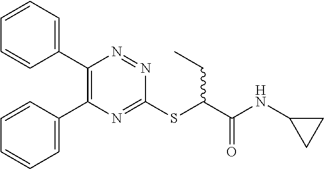 [SMILES: CCC(Sc1nnc(-c2ccccc2)c(-c2ccccc2)n1)C(=O)NC1CC1] | N-cyclopropyl-2-[(5,6- diphenyl-1,2,4-triazin-3- yl)sulfanyl]butanamide |
| B-73 | | 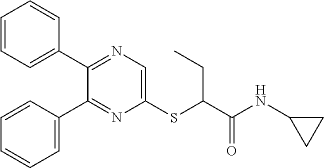 [SMILES: CCC(Sc1cnc(-c2ccccc2)c(-c2ccccc2)n1)C(=O)NC1CC1] | N-cyclopropyl-2-(5,6- diphenylpyrazin-2-yl)sulfanyl- butanamide |
| B-74 | | 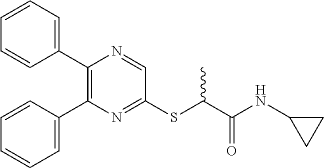 [SMILES: CC(Sc1cnc(-c2ccccc2)c(-c2ccccc2)n1)C(=O)NC1CC1] | N-cyclopropyl-2-(5,6- diphenylpyrazin-2-yl)sulfanyl- propanamide |
| B-75 | | 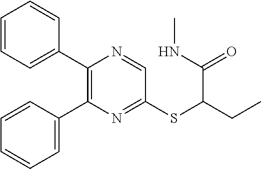 [SMILES: CCC(Sc1cnc(-c2ccccc2)c(-c2ccccc2)n1)C(=O)NC] | 2-[(5,6-Diphenylpyrazin-2- yl)sulfanyl]-N- methylbutanamide |
| B-76 | | 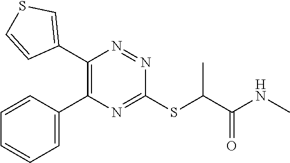 [SMILES: CNC(=O)C(C)Sc1nnc(-c2ccsc2)c(-c2ccccc2)n1] | N-methyl-2-[[5-phenyl-6-(3- thienyl)-1,2,4-triazin-3- yl]sulfanyl]propanamide |
| B-77 | | 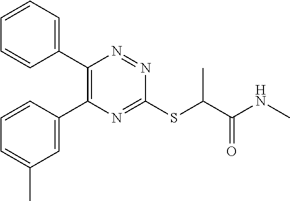 [SMILES: CNC(=O)C(C)Sc1nnc(-c2ccccc2)c(-c2cccc(C)c2)n1] | N-methyl-2-[[5-(m-tolyl)-6- phenyl-1,2,4-triazin-3- yl]sulfanyl]propanamide |

The coolants of the invention listed in Table A according to the general formulae (Va) or (VIa) are either present in neutral, uncharged form or are present in the form of their salts, for example as an acid addition salt, with inorganic or organic, mono- or polyvalent carboxylic acids, as described in detail above. In this respect, the above also applies here.

The coolants according to Table A can be present in stereoisomerically pure form or as mixtures of different stereoisomers and can therefore also be used in formulations in this way.

Surprisingly, it has been shown that the compounds according to Table A have particularly high TRPM8 activations and are therefore ideally suited as coolants.

The most preferred coolants, i.e. coolants with a particularly efficient and strong TRPM8 activation, i.e. efficient and intensive cooling effect with a low application quantity, are the compounds B-01, B-02, B-03, B-05, B-07, B-11, B14, B-15 and B-18 (TRPM8 activation ≥100%). Compounds B-01, B-02, B-03, B-05 and B-07 are particularly preferred, as they exhibit exceptionally high TRPM8 activity (TRPM8 activation ≥150%).

Due to their outstanding relative TRPM8 activation, compounds B-01 (TRPM8 activation of 262%), B-02 (TRPM8 activation of 227%), B-03 (TRPM8 activation of 221%), B-05 (TRPM8 activation of 205%), and B-07 (TRPM8 activation of 203%) are the most preferred.

Equally particularly preferred are the physiological coolants of the general formulae (III), (IV), (VII) or (VIII), selected from the group consisting of the compounds shown in Table 2:

TABLE 2
Structures according to the invention with relative TRPM8 activation in %

| Com- | TRPM8 | | |
|---|---|---|---|
| pound | activation | Structure | IUPAC name |
| A-01 | 278 | 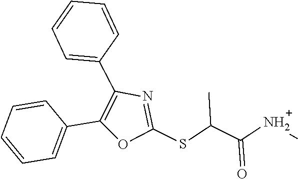 [SMILES: C[NH2+]C(=O)C(C)Sc1nc(-c2ccccc2)c(-c2ccccc2)o1] | 3-carboxy-3,5- dihydroxy-5-oxo- pentanoate;2-(4,5- diphenyloxazol-2- yl)sulfanylpropanoyl- methyl-ammonium |
| | | 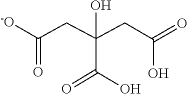 | |
| A-02 | 265 | 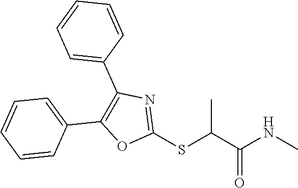 [SMILES: CNC(=O)C(C)Sc1nc(-c2ccccc2)c(-c2ccccc2)o1] | 2-(4,5- diphenyloxazol-2- yl)sulfanyl-N-methyl- propanamide |
| A-03 | 260 | 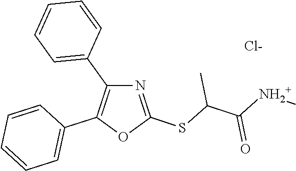 [SMILES: C[NH2+]C(=O)C(C)Sc1nc(-c2ccccc2)c(-c2ccccc2)o1 ; [Cl-]] | 2-(4,5- diphenyloxazol-2- yl)sulfanylpropanoyl- methyl- ammonium; chloride |
| A-04 | 215 | 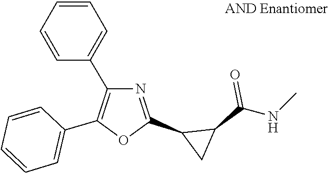 [SMILES: CNC(=O)[C@H]1C[C@H]1c1nc(-c2ccccc2)c(-c2ccccc2)o1] | rac-(1S,2R)-2-(4,5- diphenyloxazol-2-yl)- N-methyl- cyclopropane- carboxamides |
| A-05 | 190 | 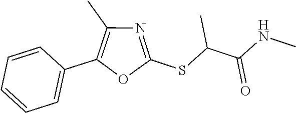 [SMILES: CNC(=O)C(C)Sc1nc(C)c(-c2ccccc2)o1] | N-methyl-2-(4- methyl-5-phenyl- oxazol-2-yl)sulfanyl- propanamide |
| A-06 | 186 | 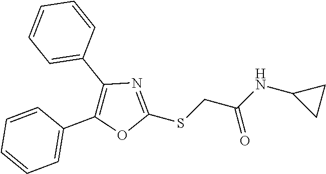 [SMILES: O=C(CSc1nc(-c2ccccc2)c(-c2ccccc2)o1)NC1CC1] | N-cyclopropyl-2- (4,5-diphenyloxazol- 2-yl)sulfanyl- acetamide |
| A-07 | 183 | 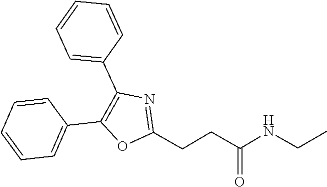 [SMILES: CCNC(=O)CCc1nc(-c2ccccc2)c(-c2ccccc2)o1] | 3-(4,5- diphenyloxazol-2-yl)- N-ethyl- propanamide |
| A-08 | 177 | 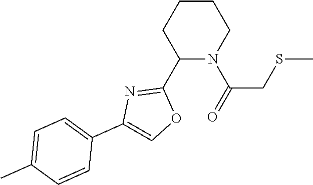 | 2-methylsulfanyl-1- [2-[4-(p-tolyl)oxazol- 2-yl]-1- piperidyllethanone |
| A-09 | 120 | 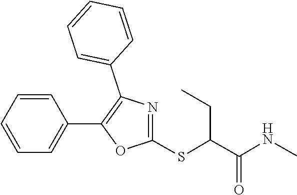 [SMILES: CCC(Sc1nc(-c2ccccc2)c(-c2ccccc2)o1)C(=O)NC] | 2-(4,5- diphenyloxazol-2- yl)sulfanyl-N-methyl- butanamide |
| A-10 | 117 | 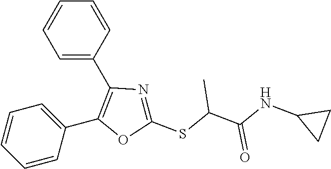 [SMILES: CC(Sc1nc(-c2ccccc2)c(-c2ccccc2)o1)C(=O)NC1CC1] | N-cyclopropyl-2- (4,5-diphenyloxazol- 2-yl)sulfanyl- propanamide |
| A-11 | 5 | 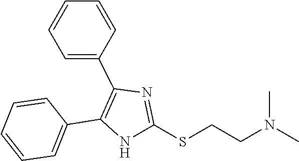 [SMILES: CN(C)CCSc1nc(-c2ccccc2)c(-c2ccccc2)[nH]1] | 2-[(4,5-diphenyl-1H- imidazol-2- yl)sulfanyl]-N,N- dimethyl-ethanamine |
| A-12 | 112 | 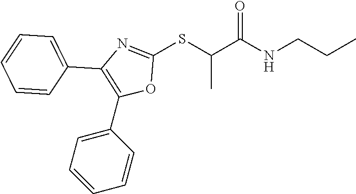 [SMILES: CCCNC(=O)C(C)Sc1nc(-c2ccccc2)c(-c2ccccc2)o1] | 2-(4,5- diphenyloxazol-2- yl)sulfanyl-N-propyl- propanamide |
| A-15 | 98 | 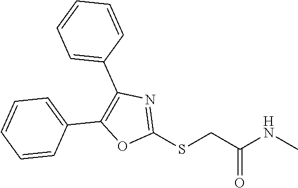 [SMILES: CNC(=O)CSc1nc(-c2ccccc2)c(-c2ccccc2)o1] | 2-(4,5- diphenyloxazol-2- yl)sulfanyl-N-methyl- acetamide |
| A-16 | 117 | 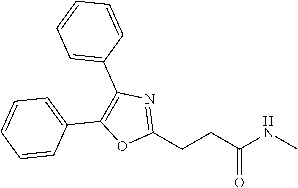 [SMILES: CNC(=O)CCc1nc(-c2ccccc2)c(-c2ccccc2)o1] | 3-(4,5- diphenyloxazol-2-yl)- N-methyl- propanamide |
| A-17 | 91 | 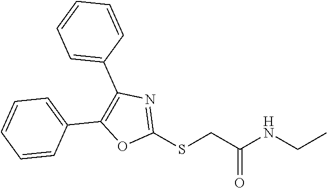 [SMILES: CCNC(=O)CSc1nc(-c2ccccc2)c(-c2ccccc2)o1] | 2-(4,5- diphenyloxazol-2- yl)sulfanyl-N-ethyl- acetamide |
| A-18 | 80 | 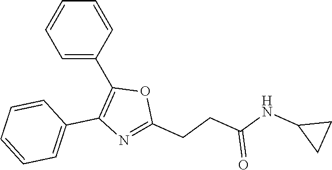 [SMILES: O=C(CCc1nc(-c2ccccc2)c(-c2ccccc2)o1)NC1CC1] | N-cyclopropyl-3- (4,5-diphenyloxazol- 2-yl)propanamide |
| A-19 | 75 | 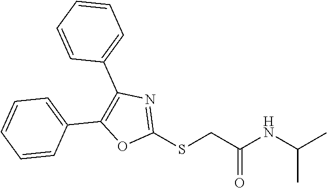 [SMILES: CC(C)NC(=O)CSc1nc(-c2ccccc2)c(-c2ccccc2)o1] | 2-(4,5- diphenyloxazol-2- yl)sulfanyl-N- isopropyl-acetamide |
| A-20 | 73 | 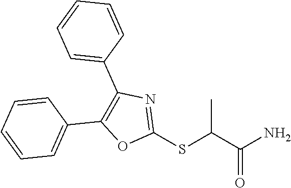 [SMILES: CC(Sc1nc(-c2ccccc2)c(-c2ccccc2)o1)C(N)=O] | 2-(4,5- diphenyloxazol-2- yl)sulfanylpropanamide |
| A-21 | 70 | 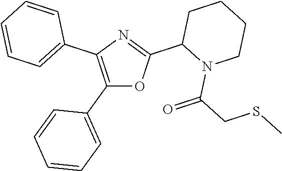 [SMILES: CSCC(=O)N1CCCCC1c1nc(-c2ccccc2)c(-c2ccccc2)o1] | 1-[2-(4,5- diphenyloxazol-2-yl)- 1-piperidyl]-2- methylsulfanyl- ethanone |
| A-22 | 69 | 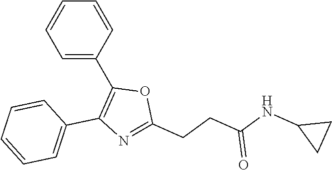 [SMILES: O=C(CCc1nc(-c2ccccc2)c(-c2ccccc2)o1)NC1CC1] | N-cyclopropyl-3- (4,5-diphenyloxazol- 2-yl)propanamide |
| A-23 | 62 | 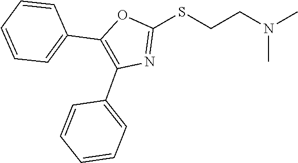 [SMILES: CN(C)CCSc1nc(-c2ccccc2)c(-c2ccccc2)o1] | 2-(4,5- diphenyloxazol-2- yl)sulfanyl-N,N- dimethyl-ethanamine |
| A-24 | 56 | 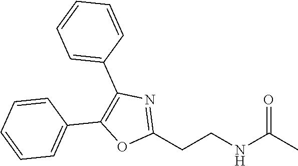 [SMILES: CC(=O)NCCc1nc(-c2ccccc2)c(-c2ccccc2)o1] | N-[2-(4,5- diphenyloxazol-2- yl)ethyl]acetamide |
| A-25 | 55 | 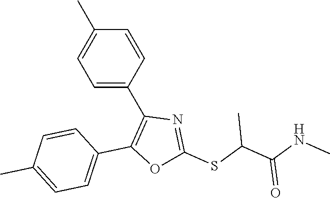 | 2-[4,5-bis(p- tolyl)oxazol-2- yl]sulfanyl-N-methyl- propanamide |
| A-26 | 51 | 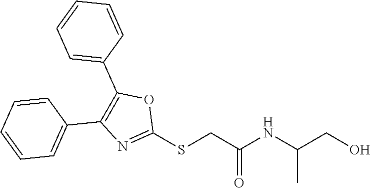 [SMILES: CC(CO)NC(=O)CSc1nc(-c2ccccc2)c(-c2ccccc2)o1] | 2-(4,5- diphenyloxazol-2- yl)sulfanyl-N-(2- hydroxy-1-methyl- ethyl)acetamide |
| A-27 | 42 | 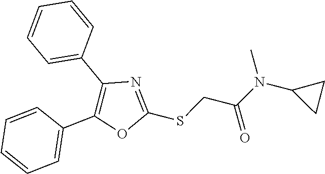 [SMILES: CN(C(=O)CSc1nc(-c2ccccc2)c(-c2ccccc2)o1)C1CC1] | N-cyclopropyl-2- (4,5-diphenyloxazol- 2-yl)sulfanyl-N- methyl-acetamide |
| A-28 | 40 | 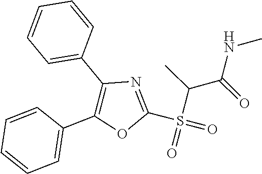 [SMILES: CNC(=O)C(C)S(=O)(=O)c1nc(-c2ccccc2)c(-c2ccccc2)o1] | 2-(4,5- diphenyloxazol-2- yl)sulfonyl-N-methyl- propanamide |
| A-29 | 36 | 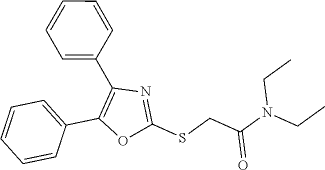 [SMILES: CCN(CC)C(=O)CSc1nc(-c2ccccc2)c(-c2ccccc2)o1] | 2-(4,5- diphenyloxazol-2- yl)sulfanyl-N,N- diethyl-acetamide |
| A-30 | 26 | 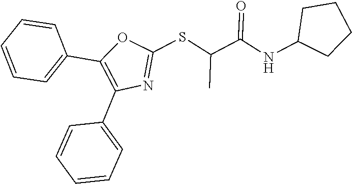 [SMILES: CC(Sc1nc(-c2ccccc2)c(-c2ccccc2)o1)C(=O)NC1CCCC1] | N-cyclopentyl-2-(4,5- diphenyloxazol-2- yl)sulfanyl- propanamide |
| A-31 | 24 | 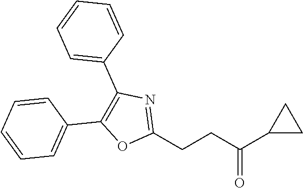 [SMILES: O=C(CCc1nc(-c2ccccc2)c(-c2ccccc2)o1)C1CC1] | 1-cyclopropyl-3-(4,5- diphenyloxazol-2- yl)propan-1-one |
| A-32 | 23 | 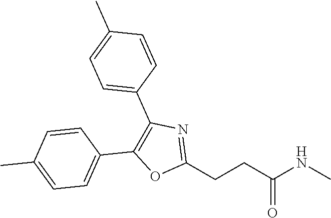 | 3-[4,5-bis(p- tolyl)oxazol-2-yl]-N- methyl-propanamide |
| A-33 | 21 | 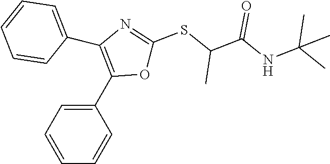 [SMILES: CC(Sc1nc(-c2ccccc2)c(-c2ccccc2)o1)C(=O)NC(C)(C)C] | N-tert-butyl-2-(4,5- diphenyloxazol-2- yl)sulfanyl- propanamide |
| A-34 | 16 | 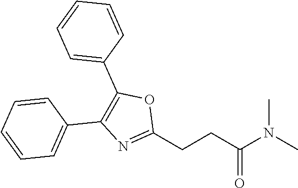 [SMILES: CN(C)C(=O)CCc1nc(-c2ccccc2)c(-c2ccccc2)o1] | 3-(4,5- diphenyloxazol-2-yl)- N,N-dimethyl- propanamide |
| A-35 | 13 | 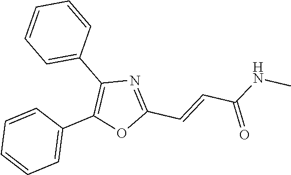 [SMILES: CNC(=O)/C=C/c1nc(-c2ccccc2)c(-c2ccccc2)o1] | (E)-3-(4,5- diphenyloxazol-2-yl)- N-methyl-prop-2- enamide |
| A-36 | 12 | 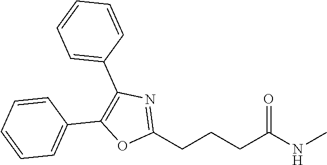 [SMILES: CNC(=O)CCCc1nc(-c2ccccc2)c(-c2ccccc2)o1] | 4-(4,5- diphenyloxazol-2-yl)- N-methyl- butanamide |
| A-37 | 7 | 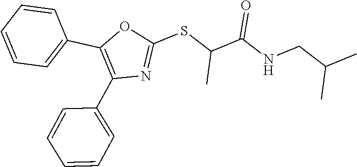 [SMILES: CC(C)CNC(=O)C(C)Sc1nc(-c2ccccc2)c(-c2ccccc2)o1] | 2-(4,5- diphenyloxazol-2- yl)sulfanyl-N- isobutyl- propanamide |
| A-38 | 6 | 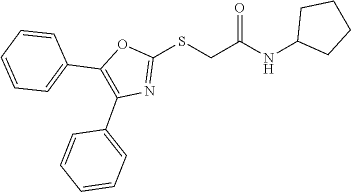 [SMILES: O=C(CSc1nc(-c2ccccc2)c(-c2ccccc2)o1)NC1CCCC1] | N-cyclopentyl-2-(4,5- diphenyloxazol-2- yl)sulfanyl- acetamide |
| A-39 | 5 | 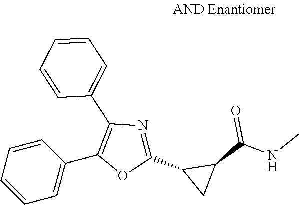 [SMILES: CNC(=O)[C@H]1C[C@@H]1c1nc(-c2ccccc2)c(-c2ccccc2)o1] | rac-(1S,2S)-2-(4,5- diphenyloxazol-2-yl)- N-methyl- cyclopropanecarboxamides |
| A-40 | 5 | 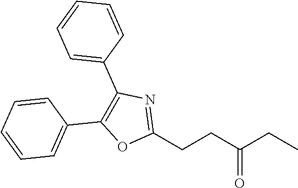 [SMILES: CCC(=O)CCc1nc(-c2ccccc2)c(-c2ccccc2)o1] | 1-(4,5- diphenyloxazol-2- yl)pentan-3-one |
| A-42 | 4 | 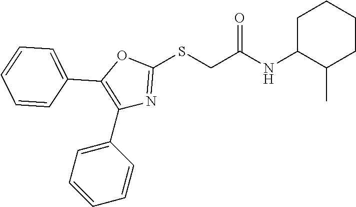 [SMILES: CC1CCCCC1NC(=O)CSc1nc(-c2ccccc2)c(-c2ccccc2)o1] | 2-(4,5- diphenyloxazol-2- yl)sulfanyl-N-(2- methylcyclohexyl) acetamide |
| A-43 | 3 | 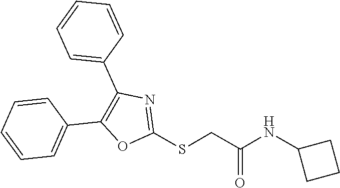 [SMILES: O=C(CSc1nc(-c2ccccc2)c(-c2ccccc2)o1)NC1CCC1] | N-cyclobutyl-2-(4,5- diphenyloxazol-2- yl)sulfanyl- acetamide |
| A-44 | 3 | 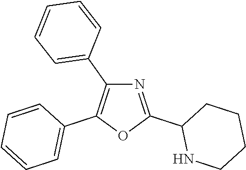 [SMILES: c1ccc(-c2nc(C3CCCCN3)oc2-c2ccccc2)cc1] | 4,5-diphenyl-2-(2- piperidyl)oxazole |
| A-45 | 2 | 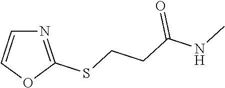 | N-methyl-3-oxazol- 2-ylsulfanyl- propanamide |
| A-46 | 2 | 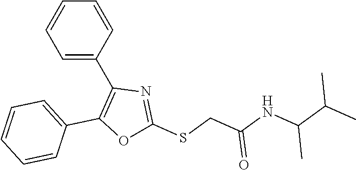 [SMILES: CC(C)C(C)NC(=O)CSc1nc(-c2ccccc2)c(-c2ccccc2)o1] | N-(1,2- dimethylpropyl)-2- (4,5-diphenyloxazol- 2-yl)sulfanyl- acetamide |
| A-47 | 2 | 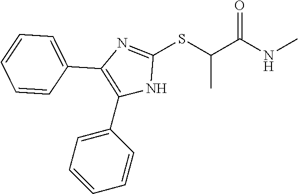 [SMILES: CNC(=O)C(C)Sc1nc(-c2ccccc2)c(-c2ccccc2)[nH]1] | 2-[(4,5-diphenyl-1H- imidazol-2- yl)sulfanyl]-N- methyl-propanamide |
| A-48 | 2 | 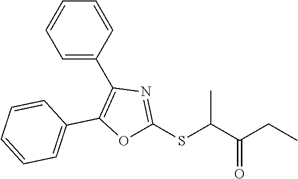 [SMILES: CCC(=O)C(C)Sc1nc(-c2ccccc2)c(-c2ccccc2)o1] | 2-(4,5- diphenyloxazol-2- yl)sulfanylpentan-3- one |
| A-49 | 2 | 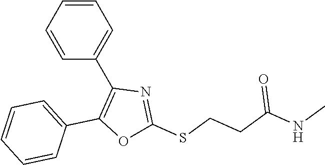 [SMILES: CNC(=O)CCSc1nc(-c2ccccc2)c(-c2ccccc2)o1] | 3-(4,5- diphenyloxazol-2- yl)sulfanyl-N-methyl- propanamide |
| A-50 | 1 | 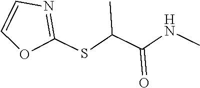 | N-methyl-2-oxazol- 2-ylsulfanyl- propanamide |
| A-51 | 1 | 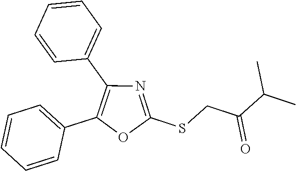 [SMILES: CC(C)C(=O)CSc1nc(-c2ccccc2)c(-c2ccccc2)o1] | 1-(4,5- diphenyloxazol-2- yl)sulfanyl-3-methyl- butan-2-one |
| A-52 | 1 | 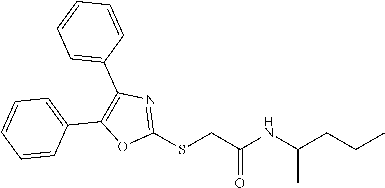 [SMILES: CCCC(C)NC(=O)CSc1nc(-c2ccccc2)c(-c2ccccc2)o1] | 2-(4,5- diphenyloxazol-2- yl)sulfanyl-N-(1- methylbutyl)acetamide |
| A-53 | 1 | 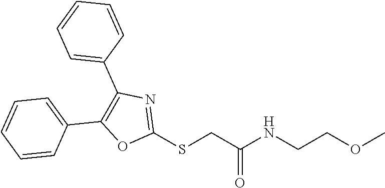 [SMILES: COCCNC(=O)CSc1nc(-c2ccccc2)c(-c2ccccc2)o1] | 2-(4,5- diphenyloxazol-2- yl)sulfanyl-N-(2- methoxyethyl)acetamide |
| A-54 | 1 | 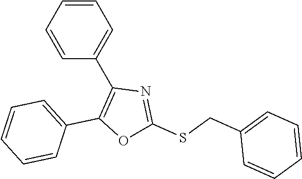 [SMILES: c1ccc(CSc2nc(-c3ccccc3)c(-c3ccccc3)o2)cc1] | 2-benzylsulfanyl-4,5- diphenyl-oxazole |
| A-55 | 1 | 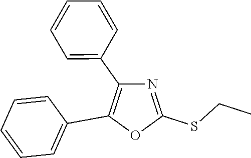 [SMILES: CCSc1nc(-c2ccccc2)c(-c2ccccc2)o1] | 2-ethylsulfanyl-4,5- diphenyl-oxazole |
| A-56 | 1 | 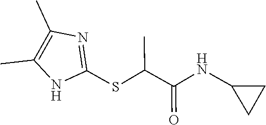 [SMILES: Cc1nc(SC(C)C(=O)NC2CC2)[nH]c1C] | N-cyclopropyl-2- [(4,5-dimethyl-1H- imidazol-2- yl)sulfanyl]propanamide |
| A-57 | 1 | 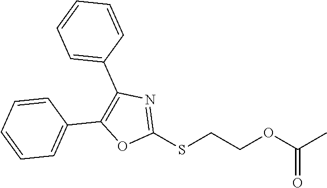 [SMILES: CC(=O)OCCSc1nc(-c2ccccc2)c(-c2ccccc2)o1] | 2-(4,5- diphenyloxazol-2- yl)sulfanylethyl acetate |
| A-58 | 1 | 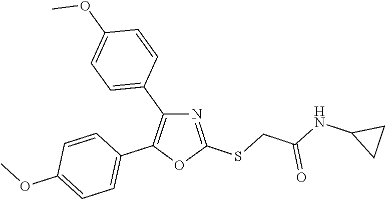 [SMILES: COc1ccc(-c2nc(SCC(=O)NC3CC3)oc2-c2ccc(OC)cc2)cc1] | 2-[4,5-bis(4- methoxyphenyl) oxazol-2-yl]sulfanyl-N- cyclopropyl- acetamide |
| A-59 | 1 | 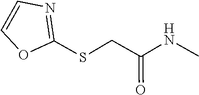 | N-methyl-2-oxazol- 2-ylsulfanyl- acetamide |
| A-60 | 0 | 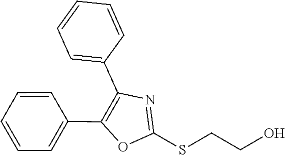 [SMILES: OCCSc1nc(-c2ccccc2)c(-c2ccccc2)o1] | 2-(4,5- diphenyloxazol-2- yl)sulfanylethanol |
| A-61 | 0 | 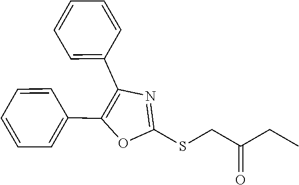 [SMILES: CCC(=O)CSc1nc(-c2ccccc2)c(-c2ccccc2)o1] | 1-(4,5- diphenyloxazol-2- yl)sulfanylbutan-2- one |
| A-62 | 0 | 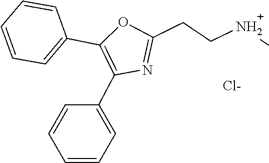 [SMILES: C[NH2+]CCc1nc(-c2ccccc2)c(-c2ccccc2)o1 ; [Cl-]] | 2-(4,5- diphenyloxazol-2- yl)ethyl-methyl- ammonium; chloride |
| A-63 | −1 | 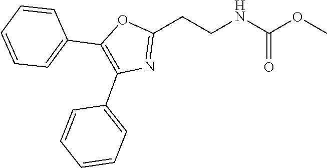 [SMILES: COC(=O)NCCc1nc(-c2ccccc2)c(-c2ccccc2)o1] | methyl N-[2-(4,5- diphenyloxazol-2- yl)ethyl]carbamate |
| A-64 | −1 | 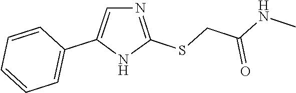 [SMILES: CNC(=O)CSc1ncc(-c2ccccc2)[nH]1] | N-methyl-2-[(5- phenyl-1H-imidazol- 2- yl)sulfanyl]acetamide |
| A-65 | −1 | 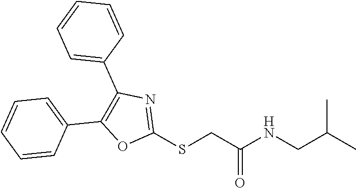 [SMILES: CC(C)CNC(=O)CSc1nc(-c2ccccc2)c(-c2ccccc2)o1] | 2-(4,5- diphenyloxazol-2- yl)sulfanyl-N- isobutyl-acetamide |
| A-66 | −1 | 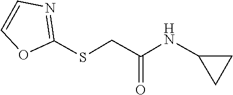 [SMILES: O=C(CSc1ncco1)NC1CC1] | N-cyclopropyl-2- oxazol-2-ylsulfanyl- acetamide |
| A-67 | −2 | 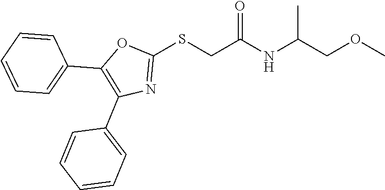 [SMILES: COCC(C)NC(=O)CSc1nc(-c2ccccc2)c(-c2ccccc2)o1] | 2-(4,5- diphenyloxazol-2- yl)sulfanyl-N-(2- methoxy-1-methyl- ethyl)acetamide |
| A-68 | −2 | 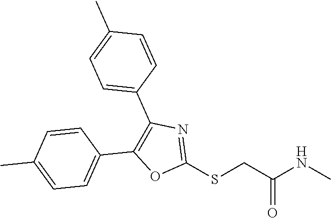 | 2-[4,5-bis(p- tolyl)oxazol-2- yl]sulfanyl-N-methyl- acetamide |
| A-69 | −2 | 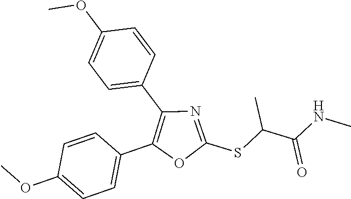 | 2-[4,5-bis(4- methoxyphenyl) oxazol-2-yl]sulfanyl-N- methyl-propanamide |
| A-70 | −2 | 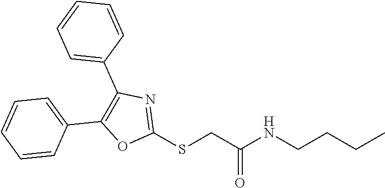 [SMILES: CCCCNC(=O)CSc1nc(-c2ccccc2)c(-c2ccccc2)o1] | N-butyl-2-(4,5- diphenyloxazol-2- yl)sulfanyl- acetamide |
| A-71 | −3 | 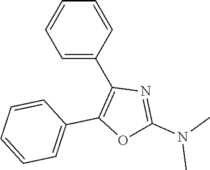 [SMILES: CN(C)c1nc(-c2ccccc2)c(-c2ccccc2)o1] | N,N-dimethyl-4,5- diphenyl-oxazol-2- amine |
| A-72 | −5 | 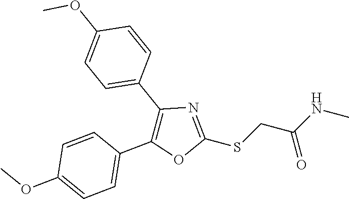 | 2-[4,5-bis(4- methoxyphenyl) oxazol-2-yl]sulfanyl-N- methyl-acetamide |
| A-73 | −5 | 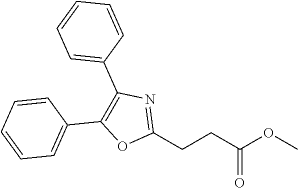 [SMILES: COC(=O)CCc1nc(-c2ccccc2)c(-c2ccccc2)o1] | methyl 3-(4,5- diphenyloxazol-2- yl)propanoate |

The coolants of the invention listed in Table 2 according to the general formulae (III), (IV), (VII) or (VIII) are either present in neutral, uncharged form or are present in the form of their salts, for example as an acid addition salt, with inorganic or organic, mono- or polyvalent carboxylic acids, as described in detail above. In this respect, the above also applies here.

The coolants according to Table 2 can be present in stereoisomerically pure form or as mixtures of different stereoisomers and can therefore also be used in formulations in this way.

Surprisingly, it has been shown that the compounds according to the invention have particularly high TRPM8 activations and are therefore ideally suited as coolants.

The most preferred coolants, i.e. coolants with a particularly efficient and strong TRPM8 activation, i.e. efficient and intensive cooling effect with a low application quantity, are the compounds A-01, A-02, A-03, A-04, A-05, A-06, A-07, A-08, A-09, A-10, A-11, A-12, A-15, A-16 and A-17 (TRPM8 activation 90%) and in particular the compounds A-01, A-02, A-03, A-04, A-05, A-06, A-07, A-08, A-09, A-10, A-11, A-12, A-15 and A-16 (TRPM8 activation 100%). Particularly preferred are compounds A-01, A-02, A-03, A-04, A-05, A-06, A-07 and A-08, which exhibit exceptionally high TRPM8 activity (TRPM8 activation 150%).

Due to their outstanding relative TRPM8 activation, compounds A-01 (TRPM8 activation of 278%), A-02 (TRPM8 activation of 265%), A-03 (TRPM8 activation of 260%), A-04 (TRPM8 activation of 215%), A-05 (TRPM8 activation of 190%), A-06 (TRPM8 activation of 186%) and A-07 (TRPM8 activation of 183%) and A-08 (TRPM8 activation of 177%) are most preferred.

The compound A-01 (oxazole derivative) is characterized in that in the general formula (VII) R1 and R2 represent a phenyl group, X represents an S atom, Y represents a branched methylene group substituted with a methyl group and Z represents an —NH—CH₃-group.

The compound A-02 (oxazole derivative) is characterized in that in the general formula (VII) R1 and R2 represent a phenyl group, X represents an S atom, Y represents a branched methylene group substituted with a methyl group and Z represents an —NH—CH₃-group.

The compound A-03 (oxazole derivative) is characterized in that in the general formula (VII) R1 and R2 represent a phenyl group, X represents an S atom, Y represents a branched methylene group substituted with a methyl group and Z represents an —NH—CH₃-group.

The compound A-04 (oxazole derivative) is characterized in that in the general formula (VII) R1 and R2 represent a phenyl group, X represents a cis-cyclopropyl group, and Z represents an —NH—CH₃-group.

The compound A-05 (oxazole derivative) is characterized in that in the general formula (VII) R1 represents a CH₃ group and R2 represents a phenyl group, X represents an S atom, Y represents a branched methylene group substituted with a methyl group, and Z represents an —NH—CH₃-group.

The compound A-06 (oxazole derivative) is characterized in that in the general formula (VII) R1 and R2 represent a phenyl group, X represents an S atom, Y represents a methylene group, and Z represents an —NH-cyclopropyl group.

The compound A-07 (oxazole derivative) is characterized in that in the general formula (VII) R1 and R2 represent a phenyl group, X and Y represent a methylene group, and Z represents an —NH—CH₂—CH₃-group.

Even more preferred are the physiological coolants of the general formulae (VIIa) or (Villa) selected from the group consisting of the compounds shown in Table B:

Table B: Structures according to the invention with relative TRPM8 activation in %

TABLE B
Structures according to the invention with relative TRPM8 activation in %

| | TRPM8 | | |
|---|---|---|---|
| Compound | activation | Structure | IUPAC name |
| A-01 | 278 | 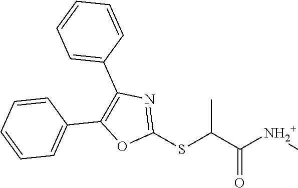 [SMILES: C[NH2+]C(=O)C(C)Sc1nc(-c2ccccc2)c(-c2ccccc2)o1] | 3-carboxy-3,5- dihydroxy-5-oxo- pentanoate;2-(4,5- diphenyloxazol-2- yl)sulfanylpropanoyl- methyl-ammonium |
| | | 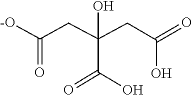 | |
| A-02 | 265 | 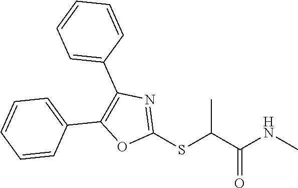 [SMILES: CNC(=O)C(C)Sc1nc(-c2ccccc2)c(-c2ccccc2)o1] | 2-(4,5- diphenyloxazol-2- yl)sulfanyl-N-methyl- propanamide |
| A-03 | 260 | 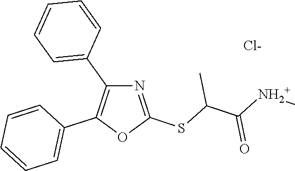 [SMILES: C[NH2+]C(=O)C(C)Sc1nc(-c2ccccc2)c(-c2ccccc2)o1 ; [Cl-]] | 2-(4,5- diphenyloxazol-2- yl)sulfanylpropanoyl- methyl- ammonium; chloride |
| A-05 | 190 | 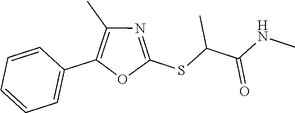 [SMILES: CNC(=O)C(C)Sc1nc(C)c(-c2ccccc2)o1] | N-methyl-2-(4- methyl-5-phenyl- oxazol-2-yl)sulfanyl- propanamide |
| A-09 | 120 | 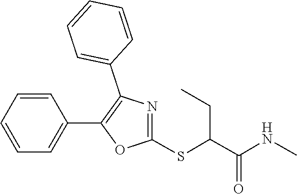 [SMILES: CCC(Sc1nc(-c2ccccc2)c(-c2ccccc2)o1)C(=O)NC] | 2-(4,5- diphenyloxazol-2- yl)sulfanyl-N-methyl- butanamide |
| A-10 | 117 | 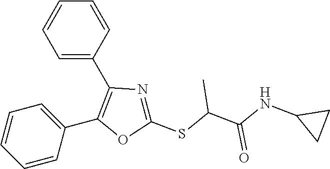 [SMILES: CC(Sc1nc(-c2ccccc2)c(-c2ccccc2)o1)C(=O)NC1CC1] | N-cyclopropyl-2- (4,5-diphenyloxazol- 2-yl)sulfanyl- propanamide |
| A-12 | 112 | 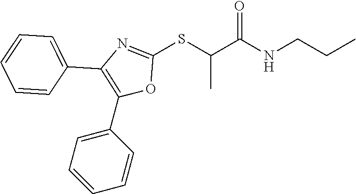 [SMILES: CCCNC(=O)C(C)Sc1nc(-c2ccccc2)c(-c2ccccc2)o1] | 2-(4,5- diphenyloxazol-2- yl)sulfanyl-N-propyl- propanamide |
| A-20 | 73 | 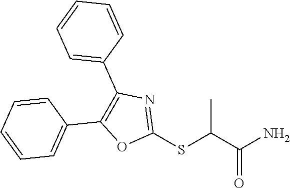 [SMILES: CC(Sc1nc(-c2ccccc2)c(-c2ccccc2)o1)C(N)=O] | 2-(4,5- diphenyloxazol-2- yl) sulfanyl- propanamide |
| A-25 | 55 | 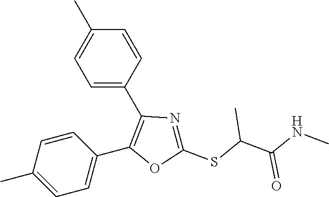 | 2-[4,5-bis(p- tolyl) oxazol-2- yl]sulfanyl-N-methyl- propanamide |
| A-28 | 40 | 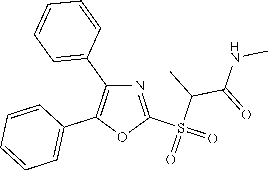 [SMILES: CNC(=O)C(C)S(=O)(=O)c1nc(-c2ccccc2)c(-c2ccccc2)o1] | 2-(4,5- diphenyloxazol-2- yl)sulfonyl-N-methyl- propanamide |
| A-30 | 26 | 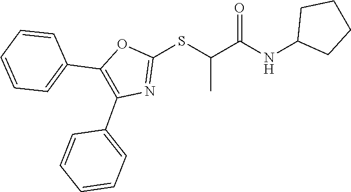 [SMILES: CC(Sc1nc(-c2ccccc2)c(-c2ccccc2)o1)C(=O)NC1CCCC1] | N-cyclopentyl-2-(4,5- diphenyloxazol-2- yl)sulfanyl- propanamide |
| A-33 | 21 | 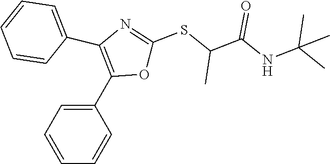 [SMILES: CC(Sc1nc(-c2ccccc2)c(-c2ccccc2)o1)C(=O)NC(C)(C)C] | N-tert-butyl-2-(4,5- diphenyloxazol-2- yl)sulfanyl- propanamide |
| A-37 | 7 | 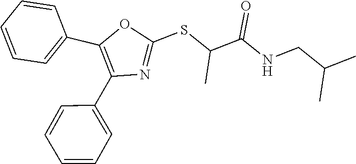 [SMILES: CC(C)CNC(=O)C(C)Sc1nc(-c2ccccc2)c(-c2ccccc2)o1] | 2-(4,5- diphenyloxazol-2- yl)sulfanyl-N- isobutyl- propanamide |
| A-47 | 2 | 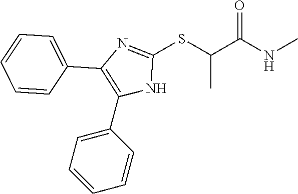 [SMILES: CNC(=O)C(C)Sc1nc(-c2ccccc2)c(-c2ccccc2)[nH]1] | 2-[(4,5-diphenyl-1H- imidazol-2- yl)sulfanyl]-N- methyl- propanamide |
| A-48 | 2 | 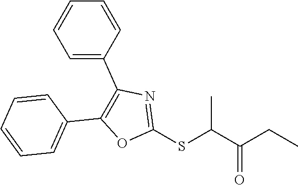 [SMILES: CCC(=O)C(C)Sc1nc(-c2ccccc2)c(-c2ccccc2)o1] | 2-(4,5- diphenyloxazol-2- yl)sulfanylpentan-3- one |
| A-50 | 1 | 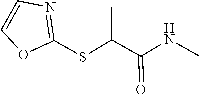 | N-methyl-2-oxazol- 2-ylsulfanyl- propanamide |
| A-56 | 1 | 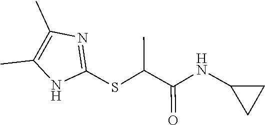 [SMILES: Cc1nc(SC(C)C(=O)NC2CC2)[nH]c1C] | N-cyclopropyl-2- [(4,5-dimethyl-1H- imidazol-2- yl)sulfanyl] propanamide |
| A-69 | -2 | 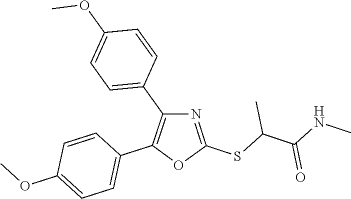 | 2-[4,5-bis(4- methoxyphenyl) oxazol-2- yl]sulfanyl-N- methyl- propanamide |

The coolants according to the invention listed in Table B according to the general formulae (VIIa) or (Villa) are either present in neutral, uncharged form or are present in the form of their salts, for example as an acid addition salt, with inorganic or organic, mono- or polyvalent carboxylic acids, as described in detail above. In this respect, the above also applies here.

The coolants according to Table B can be present in stereoisomerically pure form or as mixtures of different stereoisomers and can therefore also be used in formulations in this way.

Surprisingly, it has been shown that the compounds according to Table B have particularly high TRPM8 activations and are therefore ideally suited as coolants.

The most preferred coolants, i.e. coolants with a particularly efficient and strong TRPM8 activation, i.e. efficient and intensive cooling effect with a low application quantity, are compounds A-01, A-02, A-03, A-05, A-09, A-10 and A-12 (TRPM8 activation ≥100%). Compounds A-01, A-02, A-03 and A-05 are particularly preferred, as they exhibit exceptionally high TRPM8 activity (TRPM8 activation 150%).

Due to their outstanding relative TRPM8 activation, compounds A-01 (TRPM8 activation of 278%), A-02 (TRPM8 activation of 265%), A-03 (TRPM8 activation of 260%) and A-05 (TRPM8 activation of 190%) are the most preferred.

It has been shown that the preferred compounds B-01, B-02, B-03, B-04, B-05, B-06, B-07, B-11, B14, B-15, B-17, B-18, B-19 and B-21 and A-01, A-02, A-03, A-04, A-05, A-06, A-07, A-08, A-09, A-10, A-11, A-12, A-15, A-16 and A-17 described above have particularly high TRPM8 activities and consequently exhibit intensive and simultaneously efficient cooling effects; i.e. in order to produce an intensive cooling effect, only small amounts, in the low ppm range of about 5 ppm, of the substance according to the invention are necessary (low EC50, see experimental data below). i.e. in order to produce an intensive cooling effect, only small amounts in the low ppm range of about 5 ppm of the substance according to the invention are necessary (low EC50 values, see experimental data below). Intensive cooling effects were also demonstrated during the sensory evaluation, i.e. the tasting of the respective samples. For example, the panel lists rated the cooling effect of compound B-11 with a score of 5.3 at an application level of 5 ppm. Accordingly, the sensory evaluation of the cooling intensity, taking into account the amount of the compound used, was comparable to that determined for the cooling substance WS-3 as a reference in a concentration six times higher (amount used: 30 ppm; sensory evaluation of the cooling intensity: score 5.4).

Compounds A-2 and A-10 also show very high TRPM8 activities as well as intensely perceived cooling effects (sensory cooling intensities: score 5.4 and score 5.38 respectively) and are therefore suitable as particularly efficient coolants.

Of the compounds of the general formulae (I) to (IV) defined above, the compounds of the general formulae (V), (VI), (VII) and (VIII) are most preferred. The compounds of the general formulae (Va), (VIa), (VIIa) and (Villa) are most preferred. These coolants are characterized by a high TRPM8 activation and at the same time exhibit very high sensory cooling intensities. They cause intensive cooling effects even at low concentrations and are generally well below the EC50 reference value of 1.72 μM for the substance WS-3, as shown in the following experimental section.

Although the physiological amine coolants according to the invention are not yet known from the prior art, they can be produced according to generally known standard methods of preparative organic chemistry, which are shown in generalized form as examples in the following diagrams.

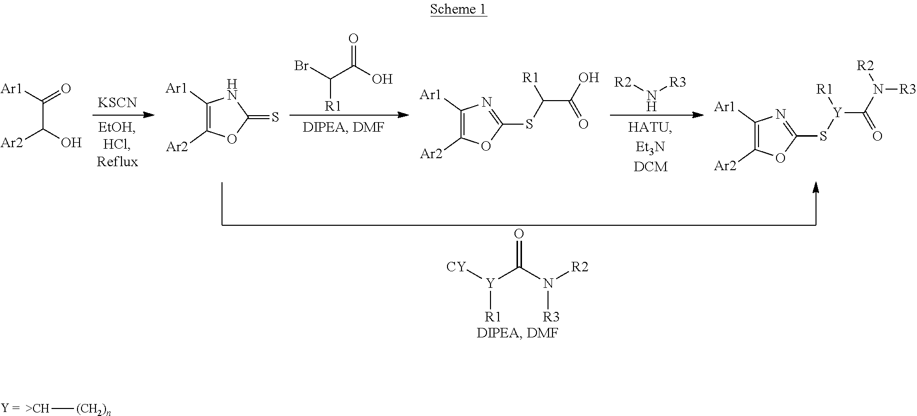

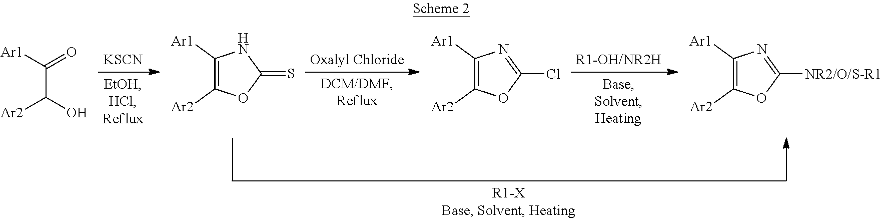

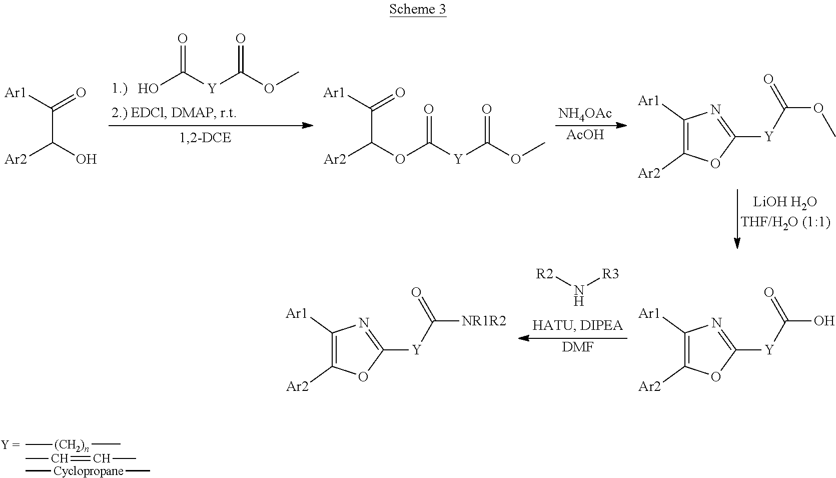

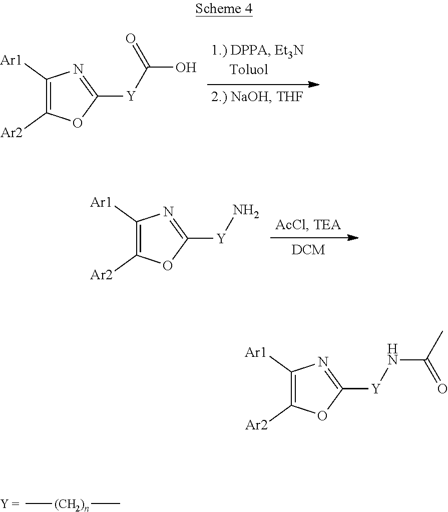

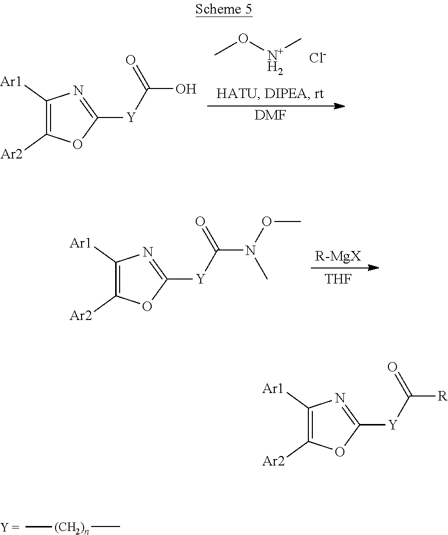

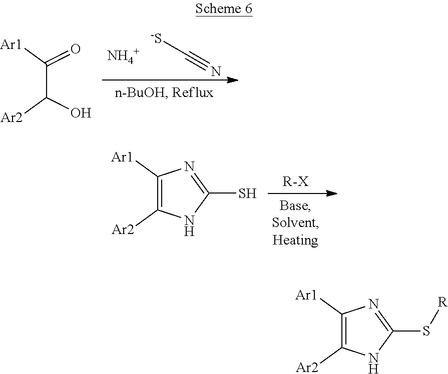

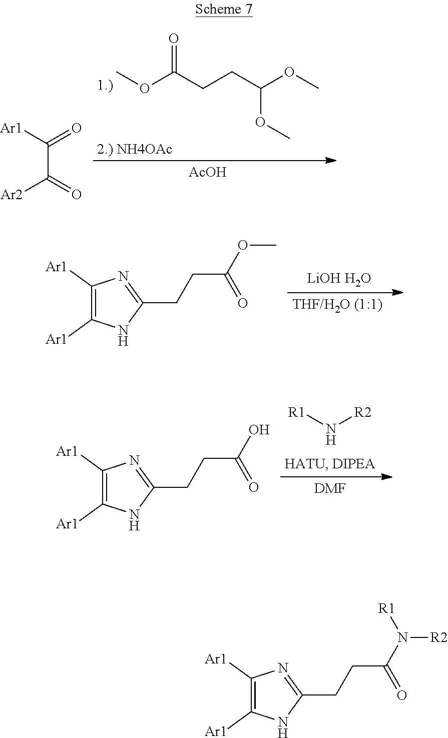

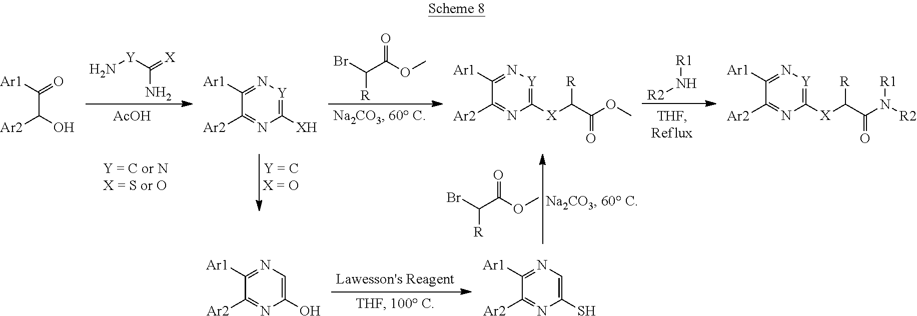

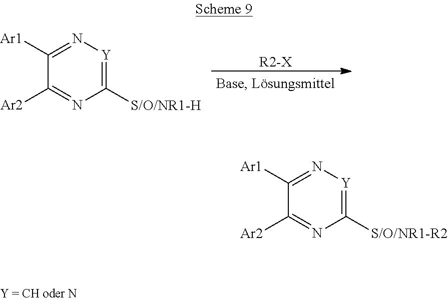

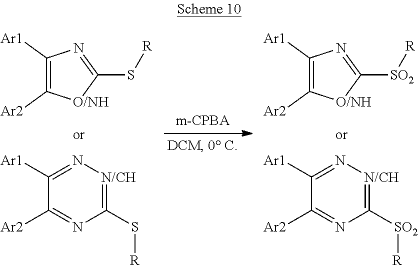

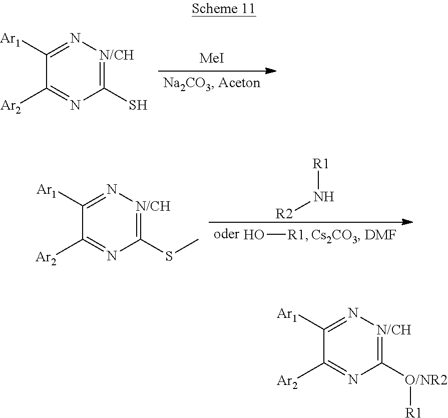

In principle, the present invention comprises all mixtures of the individual compounds of the general formulae (I) to (IV) and consequently also of the general formulae (V) to (VIII)) or (Va) to (VIII) and their use as coolants or coolant mixtures. Nevertheless, the present compounds are also suitable for mixing with other, already known coolants.

Accordingly, a further object of the invention relates to a physiological coolant mixture comprising or consisting of:
(a) one, two, three or more coolant(s) of general formulae (I) to (VIII), (Va), (VIa), (VIIa) or (VIIIa) or as listed in Table 1, Table 2, Table A or Table B and defined above; and optionally
(b) at least one other physiological coolant; and/or optionally
(c) at least one solvent.

In a preferred embodiment, the present invention relates to a coolant mixture comprising or consisting of at least one of the compounds according to the invention of general formulae (I) to (VIII), (Va), (VIa), (VIIa) or as listed in Table 1, Table 2, Table A or Table B and defined above. Optionally, the coolant mixture further comprises a further physiological coolant and optionally at least one suitable solvent.

The particular advantage of such coolant mixtures is that a synergistic enhancement of the cooling effect can be observed.

Suitable coolants forming component (b) and different from the coolant(s) forming component (a) are selected from the group consisting of menthol, menthol methyl ether (FEMA GRAS 4054), monomenthyl glutamate (FEMA GRAS 4006), menthoxy-1,2-propanediol (FEMA GRAS 3784), dimenthyl glutarate (FEMA GRAS 4604), hydroxymethylcyclohexylethanone (FEMA GRAS 4742), 2-(4-ethylphenoxy)-N-(1H-pyrazol-3-yl)-N-(thiophen-2-ylmethyl)acetamide (FEMA GRAS 4880), WS-23 (2-isopropyl-N,2,3-trimethylbutyramide, FEMA GRAS 3804), N-(4-(cyanomethyl)phenyl)-2-isopropyl-5,5-dimethylcyclohexane carboxamide (FEMA GRAS 4882), N-(3-hydroxy-4-methoxyphenyl)-2-isopropyl-5,5-dimethylcyclohexane carboxamide (FEMA GRAS 4881), N-(2-hydroxy-2-phenylethyl)-2-isopropyl-5,5-dimethylcyclohexane-1-carboxamide (FEMA GRAS 4896), 3,4-methylenedioxy cinnamic acid, (E)-3-benzo[1,3]dioxol-5-yl-N,N-diphenyl-2-propenamide (FEMA GRAS 4788), menthol propylene glycol carbonate (FEMA GRAS 3806), menthyl N-ethyloxamate, monomethyl succinate (FEMA GRAS 3810), WS-3 (N-ethyl-p-menthane-3-carboxamide, FEMA GRAS 3455), menthol ethylene glycol carbonate (FEMA GRAS 3805), WS-5 (ethyl 3-(p-menthane-3-carboxamido)acetate, FEMA GRAS 4309), WS-12 (1R,2S,5R)—N-(4-methoxyphenyl)-p-menthanecarboxamide (FEMA GRAS 4681), WS-27 (N-ethyl-2,2-diisopropylbutanamide, FEMA GRAS 4557), N-cyclopropyl-5-methyl-2-isopropylcyclohexanecarboxamide (FEMA GRAS 4693), WS-116 (N-(1,1-dimethyl-2-hydroxyethyl)-2,2-diethylbutanamide, FEMA GRAS 4603), menthoxyethanol (FEMA GRAS 4154), N-(4-cyanomethylphenyl)-p-menthanecarboxamide (FEMA GRAS 4496), N-(2-(pyridin-2-yl)ethyl)-3-p-menthanecarboxamide (FEMA GRAS 4549), N-(2-hydroxyethyl)-2-isopropy-1-2,3-dimethylbutanamide (FEMA GRAS 4602), (2S,5R)—N-[4-(2-amino-2-oxoethyl)phenyl]-p-menthanecarboxamide (FEMA GRAS 4684), N-cyclopropyl-5-methyl-2-isopropylcyclohexanecarboxamide (FEMA GRAS 4693), 2-[(2-p-menthoxy)ethoxy]-ethanol (FEMA GRAS 4718), (2,6-diethyl-5-isopropyl-2-methyltetrahydropyran (FEMA GRAS 4680), trans-4-tert-butylcyclohexanol (FEMA GRAS 4724), 2-(p-Tolyloxy)-N-(1H-pyrazol-5-yl)-N-((thiophen-2-yl)methyl)acetamide (FEMA GRAS 4809), menthone glycerol ketal (FEMA GRAS 3807 and 3808), (−)-menthoxypropane-1,2-diol, 3-(1-menthoxy)-2-methylpropane-1,2-diol (FEMA GRAS 3849), isopulegol, (+)-cis and (−)-trans-p-menthane-3,8-diol (62:38, FEMA GRAS 4053), 2,3-dihydroxy-p-menthane, 3,3,5-trimethylcyclohexanone glycerol ketal, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl-3,6,9-trioxadecanoate, (1R,2S,5R)-3-menthyl-3,6,9-trioxadecanoate, (1R,2S,5R)-3-menthyl-(2-hydroxyethoxy)acetate, (1R,2S,5R)-menthyl-11-hydroxy-3,6,9-trioxaundecanoate, cubebol (FEMA GRAS 4497), 2-isopropyl-5-methylcyclohexyl-4-(dimethylamino)-4-oxobutanoate (FEMA GRAS 4230), menthyl lactate (FEMA GRAS 3748), 6-isopropyl-3,9-dimethyl-1,4-dioxaspiro[4.5]decan-2-one (FEMA GRAS 4285), N-benzo[1,3]-dioxol-5-yl-3-p-menthanecarboxamide, N-(1-isopropyl-1,2-dimethylpropyl)-1,3-benzodioxole-5-carboxamide, N—(R)-2-oxotetrahydrofuran-3-yl-(1R,2S,5R)-p-menthane-3-carboxamide, mixture of 2,2,5,6,6-pentamethyl-2,3,6,6a-tetrahydropentalene-3a(1H)-ol and 5-(2-hydroxy-2-methylpropyl)-3,4,4-trimethylcyclopent-2-en-1-one; (2S,5R)-2-isopropyl-5-methyl-N-(2-(pyridin-4-yl)ethyl)cyclohexanecarboxamides; (1S,2S,5R)—N-(4-(cyanomethyl)phenyl)-2-isopropyl-5-methylcyclohexanecarboxamide, 1,7-isopropyl-4,5-methyl-bicyclo[2.2.2]oct-5-ene derivatives, 4-methoxy-N-phenyl-N-[2-(pyridin-2-yl)ethyl]benzamide, 4-methoxy-N-phenyl-N-[2-(pyridin-2-yl)ethyl]benzenesulfonamide, 4-chloro-N-phenyl-N-[2-(pyridin-2-yl)ethyl]benzenesulfonamide, 4-cyano-N-phenyl-N-[2-(pyridin-2-yl)ethyl]-benzenesulfonamide, 4-((benzhydrylamino)methyl)-2-methoxyphenol, 4-((bis(4-methoxyphenyl)methylamino)methyl)-2-methoxyphenol, 4-((1,2-diphenylethylamino)methyl)-2-methoxyphenol, 4-((benzhydryloxy)methyl)-2-methoxyphenol, 4-((9H-fluoren-9-ylamino)methyl)-2-methoxyphenol, 4-((benzhydrylamino)methyl)-2-ethoxyphenol, 1-(4-methoxyphenyl)-2-(1-methyl-1H-benzo[d]imidazol-2-yl)vinyl-4-methoxybenzoate, 2-(1-isopropyl-6-methyl-1H-enzo[d]imidazol-2-yl)-1-(4-methoxyphenyl)vinyl-4-methoxybenzoate, (Z)-2-(1-isopropyl-5-methyl-1H-benzo[d]imidazol-2-yl)-1-(4-methoxyphenyl)vinyl-4-methoxybenzoate, 3-alkyl-p-methan-3-ol derivatives, derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl, menthyloxamate derivatives, menthyl 3-oxocarboxylic acid esters, N-alpha-(menthanecarbonyl)amino acid amides, p-menthane carboxamide and WS-23 analogs, (−)-(1R,2R,4S)-dihydroumbellulol, p-menthane alkyloxyamide, cyclohexane derivatives, butanone derivatives, mixture of 3-menthoxy-1-propanol and 1-menthoxy-2-propanol, 1-[2-ydroxyphenyl]-4-[2-nitrophenyl]-1,2,3,6-tetrahydropyrimidin-2-one, 4-methyl-3-(1-pyrrolidinyl)-2-[5H]-furanone and mixtures thereof. FEMA stands for “Flavor and Extracts Manufacturers Association” and GRAS is defined as “Generally Regarded As Safe”. A FEMA GRAS designation means that the substance so labeled has been tested according to standard methods and is considered toxicologically safe.

In principle, all known substances with a cooling effect are suitable as component (b). For reasons of food safety, however, those compounds are preferred which have a FEMA GRAS designation or if the cooling mixture in question requires this.

A first important representative of the substances that form component (b) is monomenthyl succinate (FEMA GRAS 3810). Both the succinate and the analogous monomenthyl glutarate (FEMA GRAS 4006) are important representatives of monomenthyl esters based on di- and polycarboxylic acids.

The next important group of menthol compounds preferred in the sense of the invention comprises carbonate esters of menthol and polyols, such as glycols, glycerol or carbohydrates, such as menthol ethylene glycol carbonate (FEMA GRAS 3805=Frescolat® MGC), menthol propylene glycol carbonate (FEMA GRAS 3784=Frescolat® MPC), menthol 2-methyl-1,2-propanediol carbonate (FEMA GRAS 3849) or the corresponding sugar derivatives. Also preferred are N-(4-cyanomethylphenyl)-p-menthanecarboxamide (FEMA GRAS 4496), N-(2-(pyridin-2-yl)ethyl)-3-p-menthanecarboxamide (FEMA GRAS 4549) and (E)-3-benzo[1,3]dioxol-5-yl-N,N-diphenyl-2-propenamide (FEMA GRAS 4788) as component (b).

The menthol compounds menthyl lactate (FEMA GRAS 3748=Frescolat® ML) and in particular menthone glyceryl acetal (FEMA GRAS 3807) or menthone glyceryl ketal (FEMA GRAS 3808), which is marketed under the name Frescolat® MGA, are preferred for the purposes of the invention.

This group of compounds also includes 3-(1-menthoxy)-1,2-propanediol, also known as Coolant 10 (FEMA GRAS 3784), and 3-(1-menthoxy)-2-methyl-1,2-propanediol (FEMA GRAS 3849), which has an additional methyl group.

Menthone glyceryl acetal/ketal, menthyl lactate, menthol ethylene glycol carbonate and menthol propylene glycol carbonate, which the applicant markets under the names Frescolat® MGA, Frescolat® ML, Frecolat® MGC and Frescolat® MPC, have proven to be particularly advantageous among the above-mentioned substances.

Further preferred components (b) are shown in Table 3 below:

TABLE 3
Other suitable substances as component (b)

| Structure | Name |
|---|---|
| 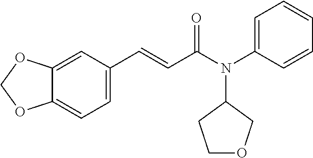 [SMILES: O=C(/C=C/c1ccc2c(c1)OCO2)N(c1ccccc1)C1CCOC1] | (E)-3-(1,3-benzodioxol-5-yl)-N-phenyl-N- tetrahydrofuran-3-yl-prop-2-enamide |
| 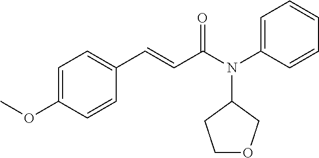 [SMILES: COc1ccc(/C=C/C(=O)N(c2ccccc2)C2CCOC2)cc1] | (E)-3-(4-methoxyphenyl)-N-phenyl-N- tetrahydrofuran-3-yl-prop-2-enamide |
| 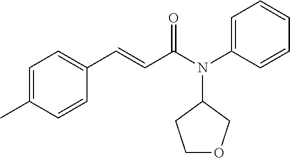 [SMILES: Cc1ccc(/C=C/C(=O)N(c2ccccc2)C2CCOC2)cc1] | (E)-N-phenyl-3-(p-tolyl)-N-tetrahydrofuran-3-yl- prop-2-enamide |
| 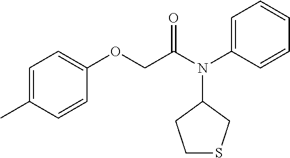 [SMILES: Cc1ccc(OCC(=O)N(c2ccccc2)C2CCSC2)cc1] | 2-(4-Methylphenoxy)-N-phenyl-N- tetrahydrothiophen-3-yl-acetamide |
| 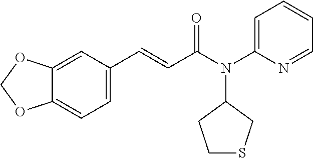 [SMILES: O=C(/C=C/c1ccc2c(c1)OCO2)N(c1ccccn1)C1CCSC1] | (E)-3-(1,3-benzodioxol-5-yl)-N-(2-pyridyl)-N- tetrahydrothiophen-3-yl-prop-2-enamide |
| 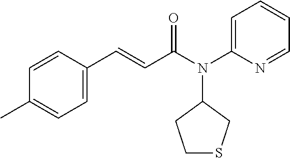 [SMILES: Cc1ccc(/C=C/C(=O)N(c2ccccn2)C2CCSC2)cc1] | (E)-3-(p-tolyl)-N-(2-pyridyl)-N- tetrahydrothiophen-3-yl-prop-2-enamide |
| 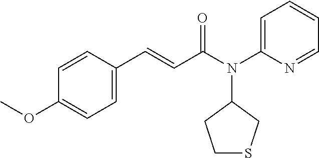 [SMILES: COc1ccc(/C=C/C(=O)N(c2ccccn2)C2CCSC2)cc1] | (E)-3-(4-methoxyphenyl)-N-(2-pyridyl)-N- tetrahydrothiophen-3-yl-prop-2-enamide |
| 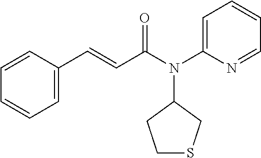 [SMILES: O=C(/C=C/c1ccccc1)N(c1ccccn1)C1CCSC1] | (E)-3-phenyl-N-(2-pyridyl)-N- tetrahydrothiophen-3-yl-prop-2-enamide |
| 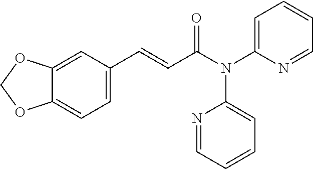 [SMILES: O=C(/C=C/c1ccc2c(c1)OCO2)N(c1ccccn1)c1ccccn1] | (E)-3-(1,3-benzodioxol-5-yl)-N,N-bis(2- pyridyl)prop-2-enamide |
| 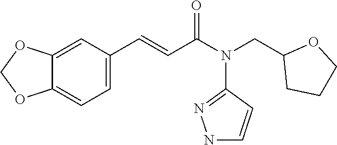 [SMILES: O=C(/C=C/c1ccc2c(c1)OCO2)N(CC1CCCO1)c1cc[nH]n1] | (E)-3-(1,3-benzodioxol-5-yl)-N-(1H-pyrazol-3- yl)-N-(tetrahydrofuran-2-ylmethyl)prop-2- enamide |
| 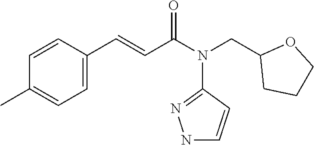 [SMILES: Cc1ccc(/C=C/C(=O)N(CC2CCCO2)c2cc[nH]n2)cc1] | (E)-3-(p-tolyl)-N-(1H-pyrazol-3-yl)-N- (tetrahydrofuran-2-ylmethyl)prop-2-enamide |
| 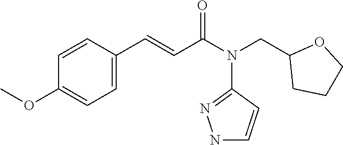 [SMILES: COc1ccc(/C=C/C(=O)N(CC2CCCO2)c2cc[nH]n2)cc1] | (E)-3-(4-methoxyphenyl)-N-(1H-pyrazol-3-yl)- N-(tetrahydrofuran-2-ylmethyl)prop-2-enamide |
| 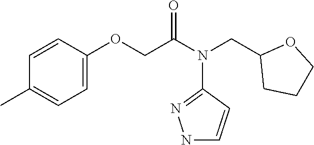 [SMILES: Cc1ccc(OCC(=O)N(CC2CCCO2)c2cc[nH]n2)cc1] | 2-(4-Methylphenoxy)-N-(1H-pyrazol-3-yl)-N- (tetrahydrofuran-2-ylmethyl)acetamide |
| 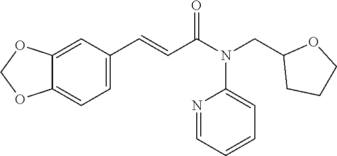 [SMILES: O=C(/C=C/c1ccc2c(c1)OCO2)N(CC1CCCO1)c1ccccn1] | (E)-3-(1,3-benzodioxol-5-yl)-N-(2-pyridyl)-N- (tetrahydrofuran-2-ylmethyl)prop-2-enamide |
| 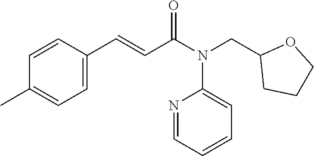 [SMILES: Cc1ccc(/C=C/C(=O)N(CC2CCCO2)c2ccccn2)cc1] | (E)-3-(p-tolyl)-N-(2-pyridyl)-N-(tetrahydrofuran- 2-ylmethyl)prop-2-enamide |
| 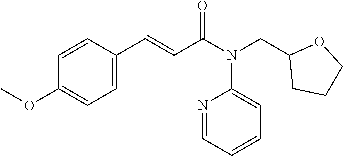 [SMILES: COc1ccc(/C=C/C(=O)N(CC2CCCO2)c2ccccn2)cc1] | (E)-3-(4-methoxyphenyl)-N-(2-pyridyl)-N- (tetrahydrofuran-2-ylmethyl)prop-2-enamide |
| 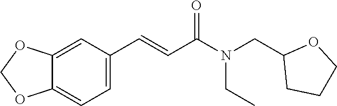 [SMILES: CCN(CC1CCCO1)C(=O)/C=C/c1ccc2c(c1)OCO2] | (E)-3-(1,3-benzodioxol-5-yl)-N-ethyl-N- (tetrahydrofuran-2-ylmethyl)prop-2-enamide |
| 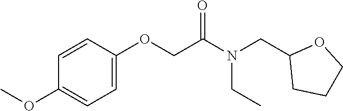 [SMILES: CCN(CC1CCCO1)C(=O)COc1ccc(OC)cc1] | N-ethyl-2-(4-methoxyphenoxy)-N- (tetrahydrofuran-2-ylmethyl)acetamide |
| 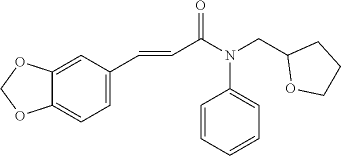 [SMILES: O=C(/C=C/c1ccc2c(c1)OCO2)N(CC1CCCO1)c1ccccc1] | (E)-3-(1,3-benzodioxol-5-yl)-N-phenyl-N- (tetrahydrofuran-2-ylmethyl)prop-2-enamide |
| 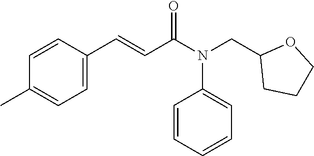 [SMILES: Cc1ccc(/C=C/C(=O)N(CC2CCCO2)c2ccccc2)cc1] | (E)-N-phenyl-3-(p-tolyl)-N-(tetrahydrofuran-2- ylmethyl)prop-2-enamide |
| 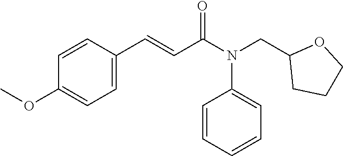 [SMILES: COc1ccc(/C=C/C(=O)N(CC2CCCO2)c2ccccc2)cc1] | (E)-3-(4-methoxyphenyl)-N-phenyl-N- (tetrahydrofuran-2-ylmethyl)prop-2-enamide |
| 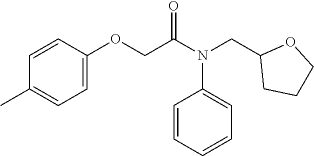 [SMILES: Cc1ccc(OCC(=O)N(CC2CCCO2)c2ccccc2)cc1] | 2-(4-methylphenoxy)-N-phenyl-N- (tetrahydrofuran-2-ylmethyl)acetamide |
| 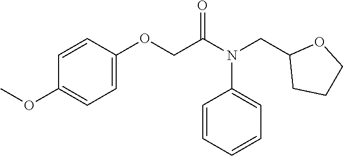 [SMILES: COc1ccc(OCC(=O)N(CC2CCCO2)c2ccccc2)cc1] | 2-(4-methoxyphenoxy)-N-phenyl-N- (tetrahydrofuran-2-ylmethyl)acetamide |
| 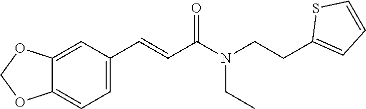 [SMILES: CCN(CCc1cccs1)C(=O)/C=C/c1ccc2c(c1)OCO2] | (E)-3-(1,3-Benzodioxol-5-yl)-N-ethyl-N-[2-(2- thienyl)ethyl]prop-2-enamid |
| 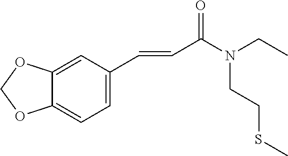 [SMILES: CCN(CCSC)C(=O)/C=C/c1ccc2c(c1)OCO2] | (E)-3-(1,3-benzodioxol-5-yl)-N-ethyl-N-(2- methylsulfanylethyl)prop-2-enamide |
| 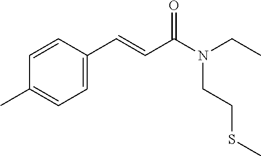 | (E)-N-Ethyl-N-(2-methylsulfanylethyl)-3-(p- tolyl)prop-2-enamide |
| 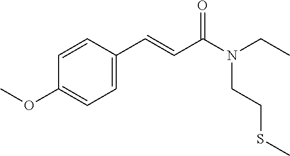 | (E)-N-Ethyl-3-(4-methoxyphenyl)-N-(2- methylsulfanylethyl)prop-2-enamide |
| 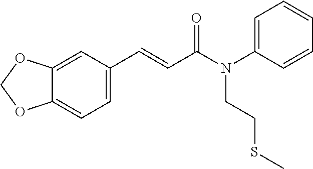 [SMILES: CSCCN(C(=O)/C=C/c1ccc2c(c1)OCO2)c1ccccc1] | (E)-3-(1,3-benzodioxol-5-yl)-N-(2- methylsulfanylethyl)-N-phenyl-prop-2-enamide |
| 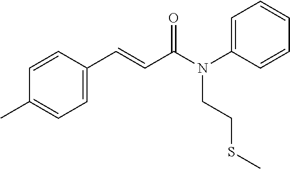 [SMILES: CSCCN(C(=O)/C=C/c1ccc(C)cc1)c1ccccc1] | (E)-N-(2-methylsulfanylethyl)-N-phenyl-3-(p- tolyl)prop-2-enamide |
| 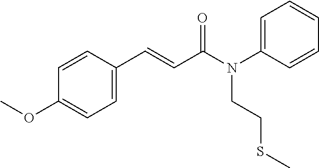 [SMILES: COc1ccc(/C=C/C(=O)N(CCSC)c2ccccc2)cc1] | (E)-3-(4-methoxyphenyl)-N-(2- methylsulfanylethyl)-N-phenyl-prop-2-enamide |
| 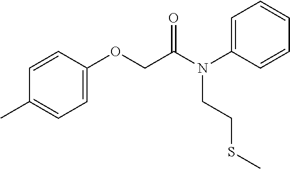 [SMILES: CSCCN(C(=O)COc1ccc(C)cc1)c1ccccc1] | 2-(4-methylphenoxy)-N-(2- methylsulfanylethyl)-N-phenyl-acetamide |
| 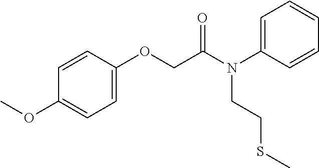 [SMILES: COc1ccc(OCC(=O)N(CCSC)c2ccccc2)cc1] | 2-(4-methoxyphenoxy)-N-(2- methylsulfanylethyl)-N-phenyl-acetamide |
| 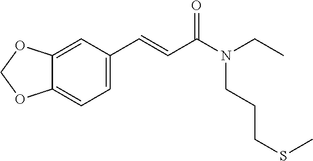 [SMILES: CCN(CCCSC)C(=O)/C=C/c1ccc2c(c1)OCO2] | (E)-3-(1,3-benzodioxol-5-yl)-N-ethyl-N-(3- methylsulfanylpropyl)prop-2-enamide |
| 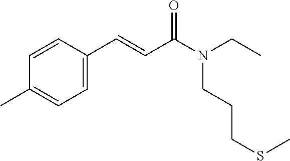 | (E)-N-Ethyl-N-(3-methylsulfanylpropyl)-3-(p- tolyl)prop-2-enamide |
| 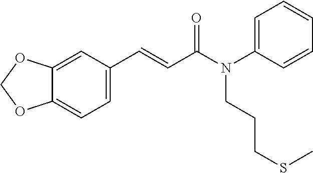 [SMILES: CSCCCN(C(=O)/C=C/c1ccc2c(c1)OCO2)c1ccccc1] | (E)-3-(1,3-benzodioxol-5-yl)-N-(3- methylsulfanylpropyl)-N-phenyl-prop-2- enamide |
| 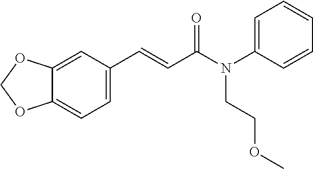 [SMILES: COCCN(C(=O)/C=C/c1ccc2c(c1)OCO2)c1ccccc1] | (E)-3-(1,3-benzodioxol-5-yl)-N-(2- methoxyethyl)-N-phenyl-prop-2-enamide |
| 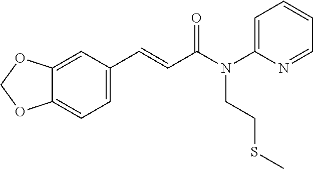 [SMILES: CSCCN(C(=O)/C=C/c1ccc2c(c1)OCO2)c1ccccn1] | (E)-3-(1,3-benzodioxol-5-yl)-N-(2- methylsulfanylethyl)-N-(2-pyridyl)prop-2- enamide |
| 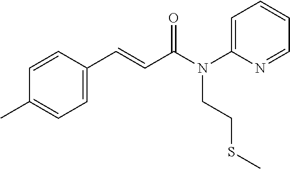 [SMILES: CSCCN(C(=O)/C=C/c1ccc(C)cc1)c1ccccn1] | (E)-N-(2-methylsulfanylethyl)-3-(p-tolyl)-N-(2- pyridyl)prop-2-enamide |
| 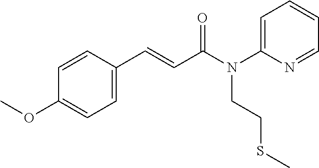 [SMILES: COc1ccc(/C=C/C(=O)N(CCSC)c2ccccn2)cc1] | (E)-3-(4-methoxyphenyl)-N-(2- methylsulfanylethyl)-N-(2-pyridyl)prop-2- enamide |
| 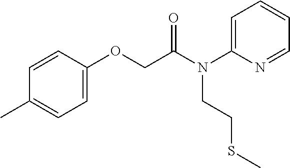 [SMILES: CSCCN(C(=O)COc1ccc(C)cc1)c1ccccn1] | 2-(4-methylphenoxy)-N-(2- methylsulfanylethyl)-N-(2-pyridyl)acetamide |
| 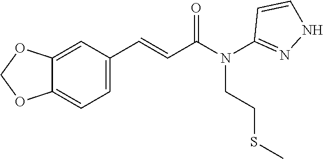 [SMILES: CSCCN(C(=O)/C=C/c1ccc2c(c1)OCO2)c1cc[nH]n1] | (E)-3-(1,3-benzodioxol-5-yl)-N-(2- methylsulfanylethyl)-N-(1H-pyrazol-3-yl)prop- 2-enamide |
| 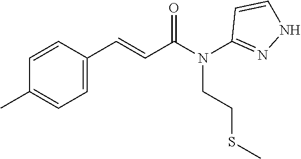 [SMILES: CSCCN(C(=O)/C=C/c1ccc(C)cc1)c1cc[nH]n1] | (E)-N-(2-methylsulfanylethyl)-3-(p-tolyl)-N-(1H- pyrazol-3-yl)prop-2-enamide |
| 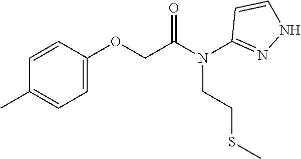 [SMILES: CSCCN(C(=O)COc1ccc(C)cc1)c1cc[nH]n1] | 2-(4-methylphenoxy)-N-(2- methylsulfanylethyl)-N-(1H-pyrazol-3- yl)acetamide |
| 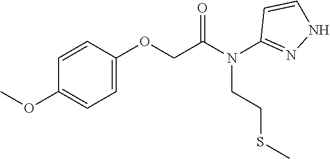 [SMILES: COc1ccc(OCC(=O)N(CCSC)c2cc[nH]n2)cc1] | 2-(4-methoxyphenoxy)-N-(2- methylsulfanylethyl)-N-(1H-pyrazol-3- yl)acetamide |

In the 1970s, menthol compounds were developed for the first time which have a C—C bond in the 3-position and of which a number of representatives can also be used in the sense of the invention. These substances are generally referred to as WS types. The basic body is a menthol derivative in which the hydroxyl group is replaced by a carboxyl group (WS-1). All other WS types are derived from this structure, such as, for example, the species WS-3, WS-4, WS-5, WS-12, WS-14, WS-23, WS-27 and WS-30, which are also preferred in the sense of the invention, or the esters or N-substituted amides of the aforementioned compounds.

Furthermore, the coolant 2-(p-tolyloxy)-N-(1H-pyrazol-5-yl)-N-((thiophen-2-yl)methyl)acetamide (FEMA GRAS 4809) is particularly preferred. Also preferred are 2-(4-ethylphenoxy)-N-(1H-pyrazol-3-yl)-N-(thiophen-2-ylmethyl)acetamide (FEMA GRAS 4880) and/or N-(3-hydroxy-4-methoxyphenyl)-2-isopropyl-5,5-dimethylcyclohexane carboxamide (FEMA GRAS 4881) and/or N-(4-(cyanomethyl)phenyl)-2-isopropyl-5,5-dimethylcyclohexane carboxamide (FEMA GRAS 4882).

Further preferred components (b), which are combined with at least one coolant according to the invention of the general formulae (I) to (VIII) or as listed in Table 1 or Table 2 and defined above, are shown in Table 4 below:

TABLE 4
Other suitable substances as component (b)

| Structure | Compound |
|---|---|
| 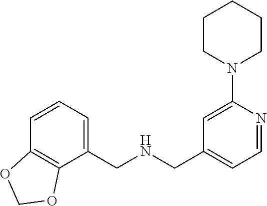 [SMILES: c1cc(CNCc2ccnc(N3CCCCC3)c2)c2c(c1)OCO2] | Compound KS1 |
| 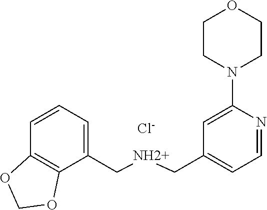 [SMILES: c1cc(C[NH2+]Cc2ccnc(N3CCOCC3)c2)c2c(c1)OCO2 ; [Cl-]] | Compound KS2 |
| 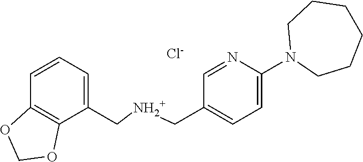 [SMILES: c1cc(C[NH2+]Cc2ccc(N3CCCCCC3)nc2)c2c(c1)OCO2 ; [Cl-]] | Compound KS3 |
| 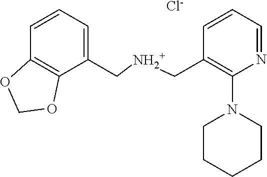 [SMILES: [Cl-] ; c1cnc(N2CCCCC2)c(C[NH2+]Cc2cccc3c2OCO3)c1] | Compound KS4 |
| 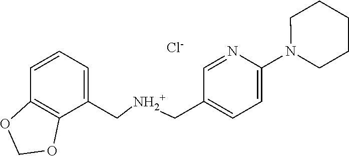 [SMILES: c1cc(C[NH2+]Cc2ccc(N3CCCCC3)nc2)c2c(c1)OCO2 ; [Cl-]] | Compound KS5 |
| 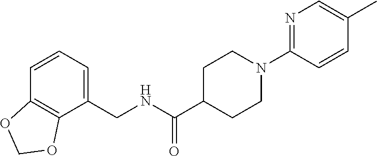 [SMILES: Cc1ccc(N2CCC(C(=O)NCc3cccc4c3OCO4)CC2)nc1] | Compound KS5 a |
| 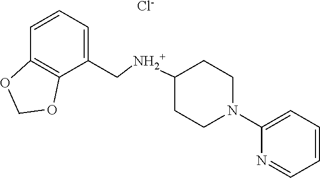 [SMILES: [Cl-] ; c1ccc(N2CCC([NH2+]Cc3cccc4c3OCO4)CC2)nc1] | Compound KS6 |
| 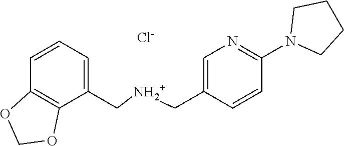 [SMILES: c1cc(C[NH2+]Cc2ccc(N3CCCC3)nc2)c2c(c1)OCO2 ; [Cl-]] | Compound KS7 |
| 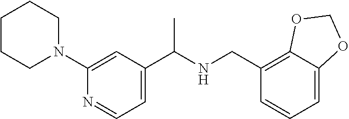 [SMILES: CC(NCc1cccc2c1OCO2)c1ccnc(N2CCCCC2)c1] | Compound KS8 |
| 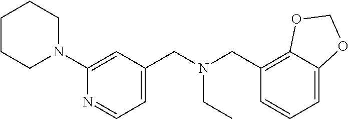 [SMILES: CCN(Cc1ccnc(N2CCCCC2)c1)Cc1cccc2c1OCO2] | Compound KS9 |
| 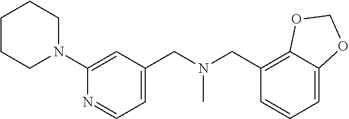 [SMILES: CN(Cc1ccnc(N2CCCCC2)c1)Cc1cccc2c1OCO2] | Compound KS10 |
| 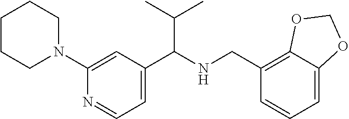 [SMILES: CC(C)C(NCc1cccc2c1OCO2)c1ccnc(N2CCCCC2)c1] | Compound KS11 |
| 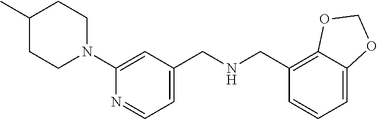 [SMILES: CC1CCN(c2cc(CNCc3cccc4c3OCO4)ccn2)CC1] | Compound KS12 |
| 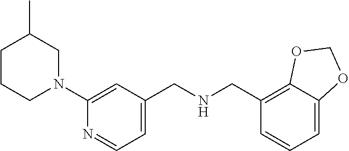 [SMILES: CC1CCCN(c2cc(CNCc3cccc4c3OCO4)ccn2)C1] | Compound KS13 |
| 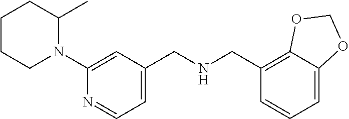 [SMILES: CC1CCCCN1c1cc(CNCc2cccc3c2OCO3)ccn1] | Compound KS14 |
| 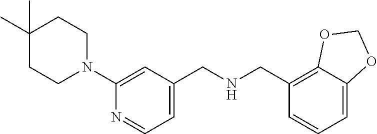 [SMILES: CC1(C)CCN(c2cc(CNCc3cccc4c3OCO4)ccn2)CC1] | Compound KS15 |
| 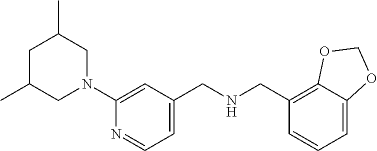 [SMILES: CC1CC(C)CN(c2cc(CNCc3cccc4c3OCO4)ccn2)C1] | Compound KS16 |
| 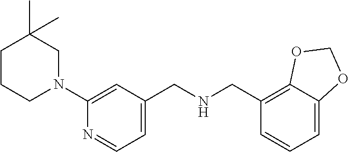 [SMILES: CC1(C)CCCN(c2cc(CNCc3cccc4c3OCO4)ccn2)C1] | Compound KS17 |
| 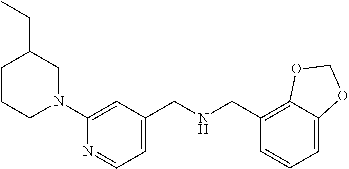 [SMILES: CCC1CCCN(c2cc(CNCc3cccc4c3OCO4)ccn2)C1] | Compound KS18 |
| 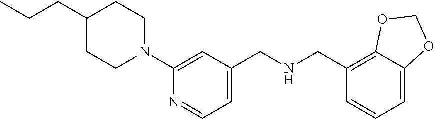 [SMILES: CCCC1CCN(c2cc(CNCc3cccc4c3OCO4)ccn2)CC1] | Compound KS18 a |
| 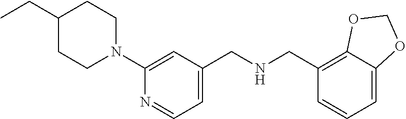 [SMILES: CCC1CCN(c2cc(CNCc3cccc4c3OCO4)ccn2)CC1] | Compound KS19 |
| 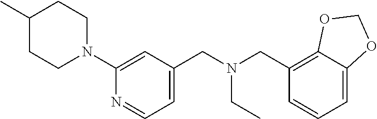 [SMILES: CCN(Cc1ccnc(N2CCC(C)CC2)c1)Cc1cccc2c1OCO2] | Compound KS19 a |
| 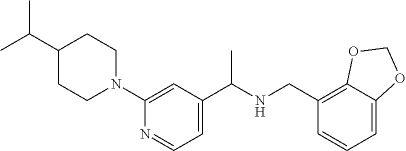 [SMILES: CC(NCc1cccc2c1OCO2)c1ccnc(N2CCC(C(C)C)CC2)c1] | Compound KS20 |
| 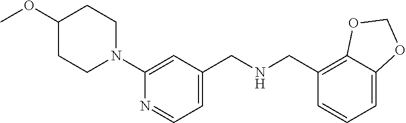 [SMILES: COC1CCN(c2cc(CNCc3cccc4c3OCO4)ccn2)CC1] | Compound KS21 |
| 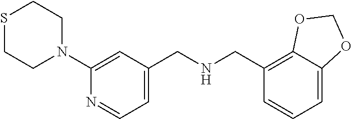 [SMILES: c1cc(CNCc2ccnc(N3CCSCC3)c2)c2c(c1)OCO2] | Compound KS22 |
| 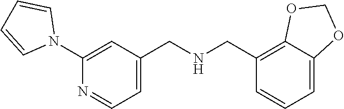 [SMILES: c1cc(CNCc2ccnc(-n3cccc3)c2)c2c(c1)OCO2] | Compound KS22 a |
| 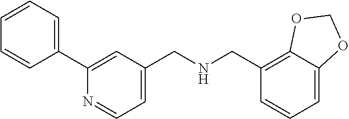 [SMILES: c1ccc(-c2cc(CNCc3cccc4c3OCO4)ccn2)cc1] | Compound KS22 b |
| 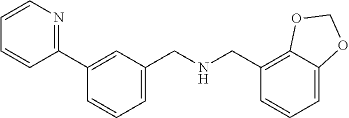 [SMILES: c1ccc(-c2cccc(CNCc3cccc4c3OCO4)c2)nc1] | Compound KS22 c |
| 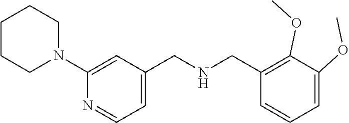 [SMILES: COc1cccc(CNCc2ccnc(N3CCCCC3)c2)c1OC] | Compound KS23 |
| 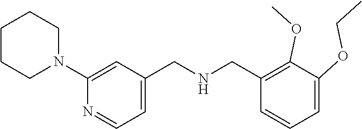 [SMILES: CCOc1cccc(CNCc2ccnc(N3CCCCC3)c2)c1OC] | Compound KS24 |
| 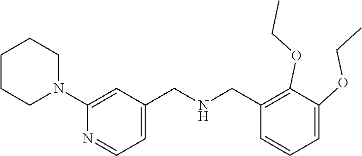 [SMILES: CCOc1cccc(CNCc2ccnc(N3CCCCC3)c2)c1OCC] | Compound KS25 |
| 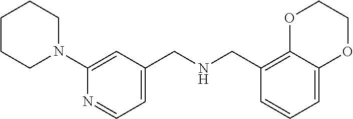 [SMILES: c1cc(CNCc2ccnc(N3CCCCC3)c2)c2c(c1)OCCO2] | Compound KS26 |
| 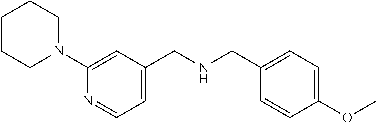 [SMILES: COc1ccc(CNCc2ccnc(N3CCCCC3)c2)cc1] | Compound KS27 |
| 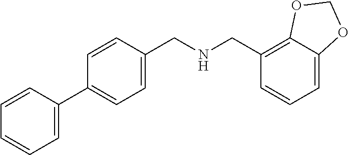 [SMILES: c1ccc(-c2ccc(CNCc3cccc4c3OCO4)cc2)cc1] | Compound KS28 |
| 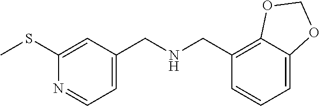 [SMILES: CSc1cc(CNCc2cccc3c2OCO3)ccn1] | Compound KS29 |
| 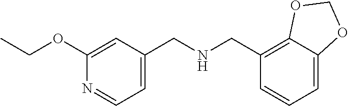 [SMILES: CCOc1cc(CNCc2cccc3c2OCO3)ccn1] | Compound KS30 |
| 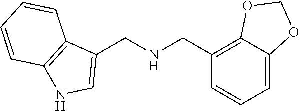 [SMILES: c1cc(CNCc2c[nH]c3ccccc23)c2c(c1)OCO2] | Compound KS31 |
| 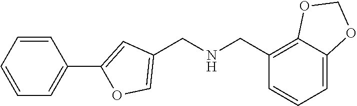 [SMILES: c1ccc(-c2cc(CNCc3cccc4c3OCO4)co2)cc1] | Compound KS31 a |
| 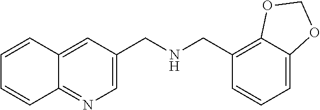 [SMILES: c1cc(CNCc2cnc3ccccc3c2)c2c(c1)OCO2] | Compound KS32 |
| 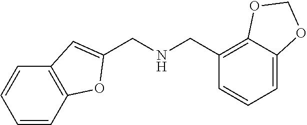 [SMILES: c1cc(CNCc2cc3ccccc3o2)c2c(c1)OCO2] | Compound KS32 a |
| 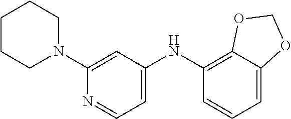 [SMILES: c1cc(Nc2ccnc(N3CCCCC3)c2)c2c(c1)OCO2] | Compound KS33 |
| 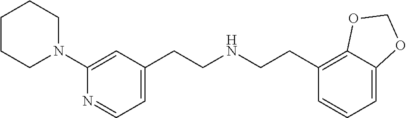 [SMILES: c1cc(CCNCCc2ccnc(N3CCCCC3)c2)c2c(c1)OCO2] | Compound KS34 |
| 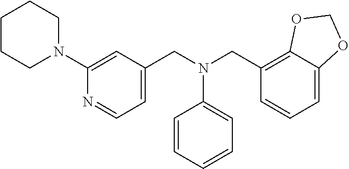 [SMILES: c1ccc(N(Cc2ccnc(N3CCCCC3)c2)Cc2cccc3c2OCO3)cc1] | Compound KS35 |
| 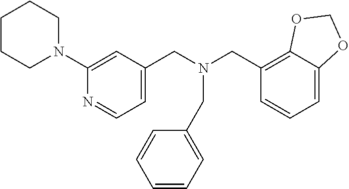 [SMILES: c1ccc(CN(Cc2ccnc(N3CCCCC3)c2)Cc2cccc3c2OCO3)cc1] | Compound KS36 |
| 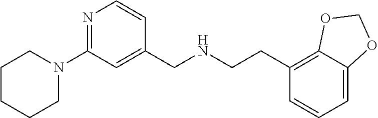 [SMILES: c1cc(CCNCc2ccnc(N3CCCCC3)c2)c2c(c1)OCO2] | Compound KS37 |
| 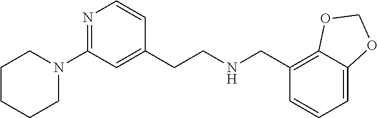 [SMILES: c1cc(CNCCc2ccnc(N3CCCCC3)c2)c2c(c1)OCO2] | Compound KS38 |
| 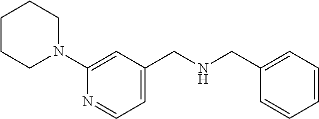 [SMILES: c1ccc(CNCc2ccnc(N3CCCCC3)c2)cc1] | Compound KS39 |
| 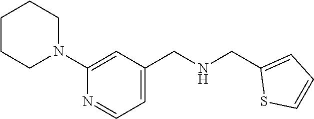 [SMILES: c1csc(CNCc2ccnc(N3CCCCC3)c2)c1] | Compound KS40 |
| 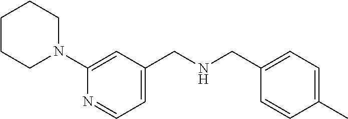 [SMILES: Cc1ccc(CNCc2ccnc(N3CCCCC3)c2)cc1] | Compound KS41 |
| 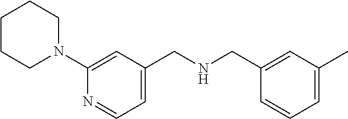 [SMILES: Cc1cccc(CNCc2ccnc(N3CCCCC3)c2)c1] | Compound KS42 |
| 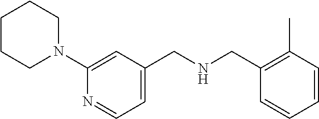 [SMILES: Cc1ccccc1CNCc1ccnc(N2CCCCC2)c1] | Compound KS43 |
| 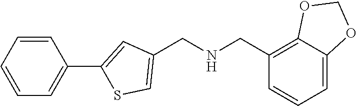 [SMILES: c1ccc(-c2cc(CNCc3cccc4c3OCO4)cs2)cc1] | Compound KS43 a |
| 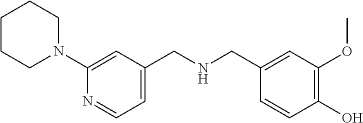 [SMILES: COc1cc(CNCc2ccnc(N3CCCCC3)c2)ccc1O] | Compound KS44 |
| 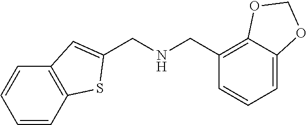 [SMILES: c1cc(CNCc2cc3ccccc3s2)c2c(c1)OCO2] | Compound KS45 |
| 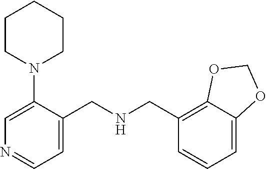 [SMILES: c1cc(CNCc2ccncc2N2CCCCC2)c2c(c1)OCO2] | Compound KS46 |
| 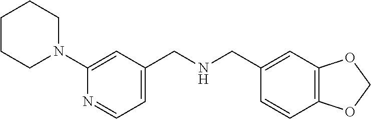 [SMILES: c1cc(CNCc2ccc3c(c2)OCO3)cc(N2CCCCC2)n1] | Compound KS47 |
| 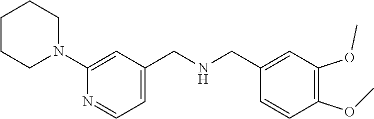 [SMILES: COc1ccc(CNCc2ccnc(N3CCCCC3)c2)cc1OC] | Compound KS48 |
| 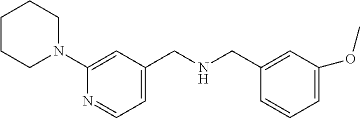 [SMILES: COc1cccc(CNCc2ccnc(N3CCCCC3)c2)c1] | Compound KS49 |
| 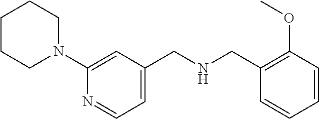 [SMILES: COc1ccccc1CNCc1ccnc(N2CCCCC2)c1] | Compound KS50 |
| 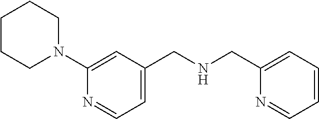 [SMILES: c1ccc(CNCc2ccnc(N3CCCCC3)c2)nc1] | Compound KS51 |
| 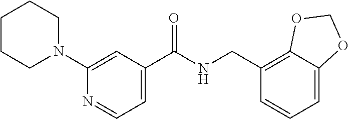 [SMILES: O=C(NCc1cccc2c1OCO2)c1ccnc(N2CCCCC2)c1] | Compound KS51 a |
| 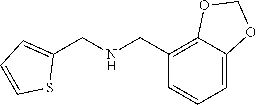 [SMILES: c1csc(CNCc2cccc3c2OCO3)c1] | Compound KS52 |
| 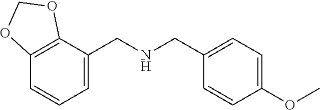 [SMILES: COc1ccc(CNCc2cccc3c2OCO3)cc1] | Compound KS53 |
| 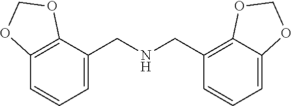 [SMILES: c1cc(CNCc2cccc3c2OCO3)c2c(c1)OCO2] | Compound KS54 |
| 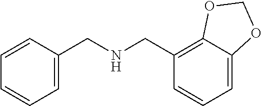 [SMILES: c1ccc(CNCc2cccc3c2OCO3)cc1] | Compound KS55 |
| 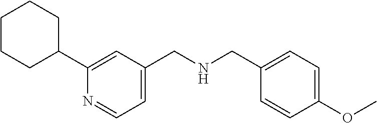 [SMILES: COc1ccc(CNCc2ccnc(C3CCCCC3)c2)cc1] | Compound KS56 |
| 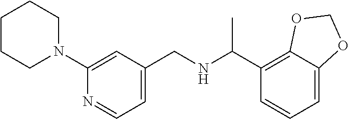 [SMILES: CC(NCc1ccnc(N2CCCCC2)c1)c1cccc2c1OCO2] | Compound KS57 |
| 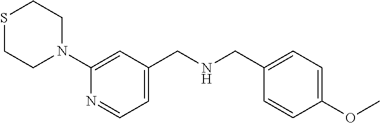 [SMILES: COc1ccc(CNCc2ccnc(N3CCSCC3)c2)cc1] | Compound KS58 |
| 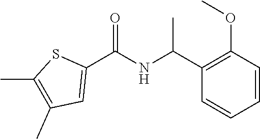 [SMILES: COc1ccccc1C(C)NC(=O)c1cc(C)c(C)s1] | Compound KS59 |
| 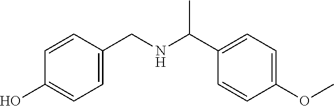 [SMILES: COc1ccc(C(C)NCc2ccc(O)cc2)cc1] | Compound KS60 |
| 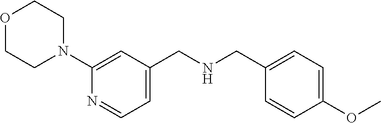 [SMILES: COc1ccc(CNCc2ccnc(N3CCOCC3)c2)cc1] | Compound KS61 |
| 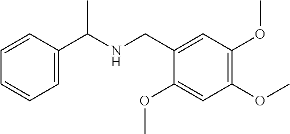 [SMILES: COc1cc(OC)c(OC)cc1CNC(C)c1ccccc1] | Compound KS62 |
| 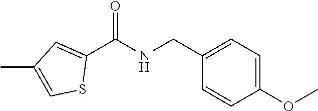 | Compound KS63 |
| 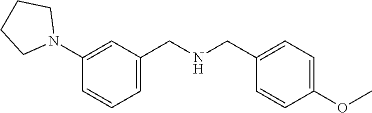 [SMILES: COc1ccc(CNCc2cccc(N3CCCC3)c2)cc1] | Compound KS64 |
| 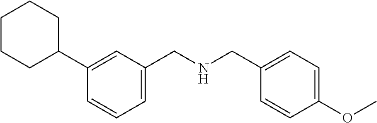 [SMILES: COc1ccc(CNCc2cccc(C3CCCCC3)c2)cc1] | Compound KS65 |
| 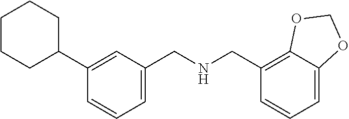 [SMILES: c1cc(CNCc2cccc3c2OCO3)cc(C2CCCCC2)c1] | Compound KS66 |
| 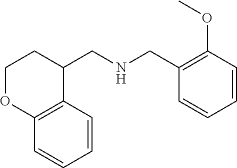 [SMILES: COc1ccccc1CNCC1CCOc2ccccc21] | Compound KS67 |
| 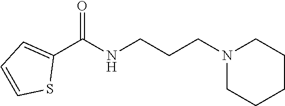 [SMILES: O=C(NCCCN1CCCCC1)c1cccs1] | Compound KS68 |
| 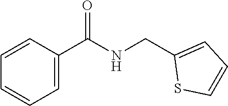 [SMILES: O=C(NCc1cccs1)c1ccccc1] | Compound KS69 |
| 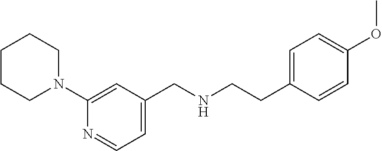 [SMILES: COc1ccc(CCNCc2ccnc(N3CCCCC3)c2)cc1] | Compound KS70 |
| 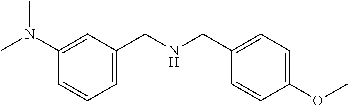 | Compound KS71 |
| 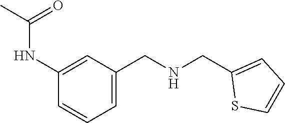 [SMILES: CC(=O)Nc1cccc(CNCc2cccs2)c1] | Compound KS72 |
| 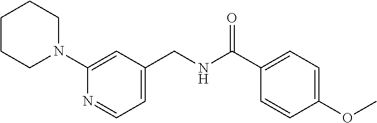 [SMILES: COc1ccc(C(=O)NCc2ccnc(N3CCCCC3)c2)cc1] | Compound KS73 |
| 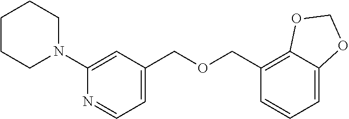 [SMILES: c1cc(COCc2ccnc(N3CCCCC3)c2)c2c(c1)OCO2] | Compound KS74 |
| 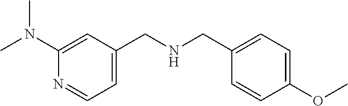 | Compound KS75 |
| 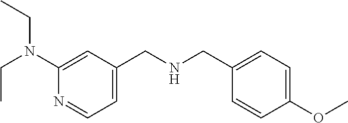 [SMILES: CCN(CC)c1cc(CNCc2ccc(OC)cc2)ccn1] | Compound KS76 |
| 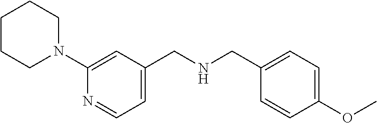 [SMILES: COc1ccc(CNCc2ccnc(N3CCCCC3)c2)cc1] | Compound KS77 |
| 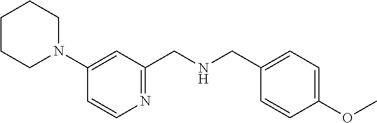 [SMILES: COc1ccc(CNCc2cc(N3CCCCC3)ccn2)cc1] | Compound KS79 |
| 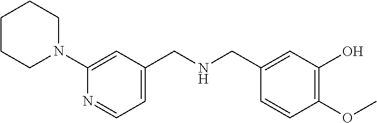 [SMILES: COc1ccc(CNCc2ccnc(N3CCCCC3)c2)cc1O] | Compound KS80 |
| 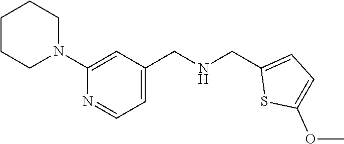 [SMILES: COc1ccc(CNCc2ccnc(N3CCCCC3)c2)s1] | Compound KS81 |
| 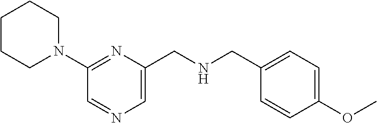 [SMILES: COc1ccc(CNCc2cncc(N3CCCCC3)n2)cc1] | Compound KS82 |
| 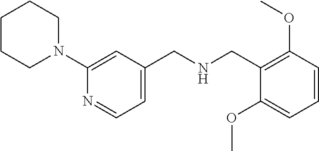 [SMILES: COc1cccc(OC)c1CNCc1ccnc(N2CCCCC2)c1] | Compound KS83 |
| 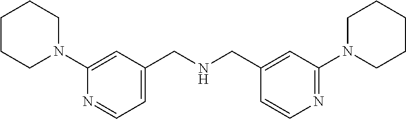 [SMILES: c1cc(CNCc2ccnc(N3CCCCC3)c2)cc(N2CCCCC2)n1] | Compound 84 |
| 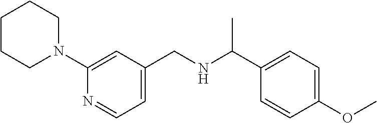 [SMILES: COc1ccc(C(C)NCc2ccnc(N3CCCCC3)c2)cc1] | Compound KS85 |
| 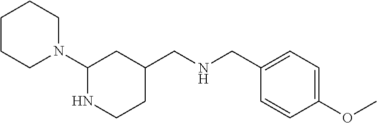 [SMILES: COc1ccc(CNCC2CCNC(N3CCCCC3)C2)cc1] | Compound KS86 |
| 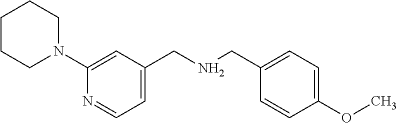 [SMILES: COc1ccc(CNCc2ccnc(N3CCCCC3)c2)cc1] | Compound KS87 |
| 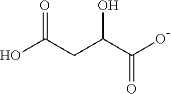 | |
| 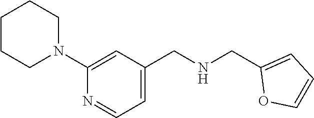 [SMILES: c1coc(CNCc2ccnc(N3CCCCC3)c2)c1] | Compound KS88 |
| 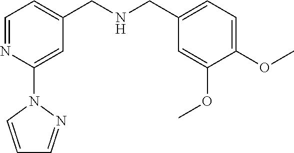 [SMILES: COc1ccc(CNCc2ccnc(-n3cccn3)c2)cc1OC] | Compound KS89 |
| 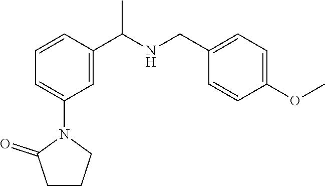 [SMILES: COc1ccc(CNC(C)c2cccc(N3CCCC3=O)c2)cc1] | Compound KS90 |

and their salts, preferably acid addition salts, with inorganic or organic acids.

The coolant mixtures according to the invention may contain components (a) and (b) in a weight ratio of about 0.1:99.9 to about 99.0:0.1, preferably from about 1:99 to about 99:1, even more preferably from about 10:90 to about 90:10, still more preferably from about 25:75 to about 75:25 and in particular from about 40:60 to about 60:40 relative to the total coolant mixture.

In order to be able to exploit and optimize the cooling effect of the coolants and to ensure easier processing in flavors and semi-finished products or other end products, the coolants must be converted into a solution before processing. However, the solubility of the coolants according to the invention is not sufficient in some cases, so that this causes problems during storage, handling or further processing.

On the one hand, the aforementioned coolants that form component (b) of the coolant mixture can act as a solvent for the coolant or coolants that form component (a) of the coolant mixture.

Advantageously, the coolant mixture according to the invention also comprises at least one solvent as a further component (c).

Individual solvents or solvent systems have proven to be advantageous, wherein the solvent is selected from the group consisting of: benzyl alcohol, 2-phenylethanol, benzyl benzoate, diethyl succinate, triethyl citrate, triacetin, ethanol, peppermint oil, anethole, optamint, propylene glycol, phenoxyethanol and mixtures thereof.

Optamint, for example, is a mixture of more than 50 different natural essential oils and natural or nature-identical flavorings. Optamints have variable compositions of different (partially fractionated) oils, which are preferably a mixture of, for example, different peppermint oils and spearmint oils, as well as eucalyptus globulus oil, star anise oil, menthol, menthone, isomenthone, menthyl acetate, anethole, eucalyptol etc. An exact reproduction of the composition of the Optamints is therefore not possible. The Optamint® product series is commercially available from Symrise AG.

For example, benzyl alcohol or 2-phenylethanol or benzyl benzoate can be used as solvents in the coolant mixture according to the invention.

The use of benzyl alcohol or 2-phenyethanol or benzyl benzoate can be used, for example, to bring the coolants according to the invention into solution and also to obtain a stable solution, i.e. coolant mixture, for appropriate storage.

Solvent systems, i.e. solvent combinations of two or more solvents, can also be used to dissolve the coolants according to the invention. Particularly with regard to the subsequent area of application, the use of solvents, which can also have a cooling effect, can —save a further step in the (final-) production step.

In an exemplary embodiment, the solvent in the coolant mixture is therefore a binary system of two solvent substances selected from the group consisting of benzyl alcohol, 2-phenylethanol, benzyl benzoate, diethyl succinate, triethyl citrate, triacetin, ethanol, peppermint oil, anethole, optamint, propylene glycol, phenoxyethanol and further coolants as described above as component (b).

For example, binary solvent systems of benzyl alcohol and a further substance selected from the group consisting of 2-phenylethanol, benzyl benzoate, diethyl succinate, triethyl citrate, triacetin, ethanol, peppermint oil, anethole, optamint, propylene glycol, phenoxyethanol and further coolants as described above as component (b) are suitable according to the present invention.

Binary solvent combinations or mixtures which contain or consist of, for example, benzyl alcohol with a further solvent are also suitable. Binary solvent combinations or mixtures selected from the following are also suitable in the present case: Benzyl alcohol and 2-phenylethanol, benzyl alcohol and benzyl benzoate, benzyl alcohol and diethyl succinate, benzyl alcohol and triethyl citrate, benzyl alcohol and triacetin, benzyl alcohol and ethanol, benzyl alcohol and peppermint oil, benzyl alcohol and anethole, benzyl alcohol and optamint, benzyl alcohol and propylene glycol, benzyl alcohol and menthol, benzyl alcohol and menthyl lactate (Frescolat® ML), benzyl alcohol and menthol propylene glycol carbonate (Frescolat® MPC), benzyl alcohol and menthol ethylene glycol carbonate (Frescolat® MGC), benzyl alcohol and menthone glyceryl acetal (Frescolat® MGA), benzyl alcohol and menthane carboxylic acid esters and amides.

The following binary solvent combinations or mixtures are also suitable-mixtures are also suitable: 2-phenylethanol and menthol propylene glycol carbonate (Frescolat® MPC), diethyl succinate and 2-phenylethanol, triacetin and benzyl benzoate, triethyl citrate and triacetin, 2-phenylethanol and peppermint oil, 2-phenylethanol and optamint, Anethole and triacetin, peppermint oil and menthyl lactate (Frescolat® ML), triacetin and menthone glyceryl acetal (Frescolat® MAG), optamint and menthyl lactate (Frescolat® ML), triethyl citrate and menthol ethylene glycol carbonate (Frescolat® MGC).

Suitable coolant mixtures within the meaning of the present invention therefore contain as solvent (c), for example, a binary solvent combination or mixture as described above.

The binary solvent mixtures according to the present invention have, for example, the following ratios: Solvent (1): solvent (2) in a ratio of from 10:1 to 1:10, preferably in a ratio of from 8:2 to 2:8, even more preferably from 6:4 to 4:6 and most preferably in a ratio of 5:5.

The above-mentioned suitable binary solvent mixtures can dissolve the coolants according to the invention and, depending on the solvent or combination of said solvents, -stably hold the coolants in-solution in an amount of 2 wt. % to 50 wt.-%, preferably 5 wt. % to 40 wt. % and further preferably 5 wt. % to 20 wt. % over a wide range.

In a further exemplary embodiment, the solvent or solvent system for the coolants according to the invention is a ternary system of three solvents selected from the group consisting of benzyl alcohol, 2-phenylethanol, benzyl benzoate, diethyl succinate, triethyl citrate, triacetin, ethanol, peppermint oil, anethole, optamint, propylene glycol, phenoxyethanol and further coolants as described above as component (b).

For example, ternary solvent combinations or mixtures of benzyl alcohol and two other substances selected from the group consisting of 2-phenylethanol, benzyl benzoate, diethyl succinate, triethyl citrate, triacetin, ethanol, peppermint oil, anethole, optamint, propylene glycol, phenoxyethanol and other coolants, as also described above as component (b), are suitable for this purpose.

Ternary solvent combinations or mixtures are suitable in this respect. -suitable are ternary solvent combinations or mixtures which contain or consist of, for example, benzyl alcohol with two further solvents, the two further solvents being selected from the group consisting of: 2-phenylethanol and benzyl benzoate, 2-phenylethanol and diethyl succinate, triethyl citrate and triacetin, triacetin and ethanol, triacetin and peppermint oil, menthol ethylene glycol carbonate (Frescolat® MGC) and anethole, 2-phenylethanol and optamint, Optamint and propylene glycol, diethyl succinate and menthol, triacetin and menthyl lactate (Frescolat® ML), anethole and menthol propylene glycol carbonate (Frescolat® MPC), triacetin and menthol ethylene glycol carbonate (Frescolat® MGC), 2-phenylethanol and menthone glyceryl acetal (Frescolat® MGA), 2-phenylethanol and menthone carboxylic acid esters and amides, 2-phenylethanol and menthol propylene glycol carbonate (Frescolat® MPC), triacetin and benzyl benzoate, 2-phenylethanol and peppermint oil, Anethole and triacetin, peppermint oil and menthyl lactate (Frescolat® ML), triacetin and menthone glyceryl acetal (Frescolat® MGA), optamint and menthyl lactate (Frescolat® ML), triethyl citrate and menthol ethylene glycol carbonate (Frescolat® MGC). Benzyl benzoate and menthol ethylene glycol carbonate (Frescolat® MGC), 2-phenylethanol and triethyl citrate, triethyl citrate and diethyl succinate, peppermint oil and menthyl lactate (Frescolat® ML), and ethanol and menthyl lactate (Frescolat® ML).

The following ternary solvent combinations or mixtures are also suitable, for example:
Triethyl citrate, triacetin, menthyl lactate (Frescolat® ML),
Triacetin, 2-phenylethanol and peppermint oil,
2-phenylethanol, optamint and peppermint oil,
2-phenylethanol, triacetin and Optamint,
Anethole, benzyl alcohol and triacetin,
2—Phenylethanol, benzyl benzoate and optamint
2—Phenylethanol, diethyl succinate and Optamint
Triethyl citrate, triacetin and peppermint oil,
Optamint, triacetin and ethanol,
Triacetin, menthol ethylene glycol carbonate (Frescolat® MGC) and anethole,
2-phenylethanol, Optamint and propylene glycol,
Diethyl succinate, triacetin and menthol,
Triacetin, benzyl benzoate and menthyl lactate (Frescolat® ML),
Anethole, menthol propylene glycol carbonate (Frescolat® MPC) and menthol ethylene glycol carbonate (Frescolat® MGC),
Triacetin, 2-phenylethanol and menthone glyceryl acetal (Frescolat® MGA),
Peppermint oil, 2-phenylethanol and menthanecarboxylic acid esters and amides,
Triacetin, 2-phenylethanol and menthol propylene glycol carbonate (Frescolat® MPC),
Menthyl lactate (Frescolat® ML), 2-phenylethanol and peppermint oil,
Anethole, triacetin and menthone glyceryl acetal (Frescolat® MGA),
Optamint, benzyl benzoate and menthyl lactate (Frescolat® ML), and
benzyl benzoate, triethyl citrate and menthol ethylene glycol carbonate (Frescolat® MGC).

The ternary solvent mixtures according to the present invention have, for example, the following ratios: Solvent (1): solvent (2): solvent (3) in a ratio of in a ratio of from 10:1:15 to 5:1:3, or in a ratio of from 4:1:7 to 7:1:4, or in a ratio of from 2:2:4 to 4:4:2.

The above-mentioned suitable ternary solvent mixtures showed themselves to be particularly good in their ability to dissolve the coolants according to the invention and to keep the coolants stable in solution in an amount of 2 wt. % to 50 wt. %, preferably 5 wt. % to 40 wt. % and further preferably 5 wt. % to 20 wt. % over a wide range, depending on the solvent or combination of said solvents.

This has the advantage that the coolant(s) according to the invention can thus be presented in a variable amount suitable for the final formulation, so that the range of coolant mixtures in which the coolant(s) is/are present in solution is broad.

In a further suitable embodiment, the solvent or solvent system for the coolants according to the invention is a quaternary system of four solvents selected from the group consisting of: Benzyl alcohol, 2-phenylethanol, benzyl benzoate, diethyl succinate, triethyl citrate, triacetin, ethanol, peppermint oil, anethole, optamint, propylene glycol, phenoxyethanol and other coolants as described further above as component (b).

Suitable solvent combinations are, for example, quaternary solvent combinations of benzyl alcohol and three other substances selected from the group consisting of: 2-phenylethanol, benzyl benzoate, diethyl succinate, triethyl citrate, triacetin, ethanol, peppermint oil, anethole, optamint, propylene glycol, phenoxyethanol and other coolants, as described above as component (b).

Suitable quaternary solvent combinations or mixtures are those which contain or consist of, for example, benzyl alcohol with three further solvents, the three further solvents being selected from the group consisting of:
2-phenylethanol, triethyl citrate and triacetin,
Peppermint oil, 2-phenylethanol and triethyl citrate,
Triethyl citrate, menthyl lactate (Frescolat® ML) and diethyl succinate Triethyl citrate, triacetin and anethole,
2—Phenylethanol, Triacetin, and Optamint,
Peppermint oil, benzyl alcohol and menthyl lactate (Frescolat® ML),
Optamint, ethanol and menthyl lactate (Frescolat® ML),
2-phenylethanol, benzyl benzoate and diethyl succinate,
Triethyl citrate, triacetin and ethanol,
Peppermint oil, anethole and optamint,
2-phenylethanol, benzyl benzoate and propylene glycol,
2-phenylethanol, benzyl benzoate and menthol propylene glycol carbonate (Frescolat® MPC),
Triethyl citrate, Optamint and ethanol,
Triacetin, benzyl benzoate and menthoxy-2-methyl-1,2-propanediol,
Menthone glyceryl acetal (Frescolat® MGA), triacetin and anethole.

The following quaternary solvent combinations and solvent mixtures are also suitable: Anethole, triacetin, peppermint oil and menthol ethylene glycol carbonate (Frescolat® MGC), Triacetin, ethanol, 2-phenylethanol and peppermint oil, 2-phenylethanol, optamint, diethyl succinate and peppermint oil, Anethole, 2-phenylethanol, benzyl alcohol and triacetin.

The above-mentioned suitable quaternary solvent mixtures showed themselves to be particularly good in their ability to dissolve the coolants according to the invention and to-keep the coolants-stable in solution in an amount of 2 wt. % to 50 wt.-%, preferably 5 wt. % to 40 wt. % and further preferably 5 wt. % to 20 wt. %, over a wide range, depending on the solvent or combination of said solvents.

This has the advantage that the coolant(s) according to the invention can thus be presented in a variable amount suitable for the final formulation, so that the range of coolant mixtures in which the coolant(s) is/are present in solution is broad.

The coolant mixtures according to the invention preferably contain or consist of component (a) and/or component (b) in an amount of from 2% to 20% by weight-, preferably from 2%-to 10% by weight-, even more preferably from 5% to 10% by weight, most preferably from 5%-to 8% by weight-, and/or component (c) in an amount of from −80% to-98% by weight-, preferably 90% to 98% by weight, even more preferably from 90% to 95% by weight, most preferably from 92% to 95% by weight, based on the total coolant mixture.% to-98% by weight, even more preferably from 90-% to 95% by weight, most preferably from 92% to 95% by weight, based on the total coolant mixture, with the proviso that components (a) and/or (b) and/or (c) together give 100% by weight.

This composition of the coolant mixture according to the invention is particularly advantageous, as it allows the amount of coolant(s) in the final formulation to be controlled.

Preferably, the final product contains the coolant(s) in an amount of about 0.00001 wt.-% to 50 wt.-%, preferably 0.0001 wt.-% to 10 wt.-%, more preferably 0.001 wt.-% to 5 wt.-%, and even more preferably 0.005 wt.-% to 1 wt.-% or 0.1 wt.-% to 20 wt.-%, more preferably 0.5 wt.-% to 15 wt.-% or 1 wt.-% to 5 wt.-% based on the weight of the final product, in particular in the case of oral care compositions.

Suitable coolant mixtures according to the invention have, for example, the following composition or consist, for example, of:
5-10% by weight of coolant(s) in 95-90% by weight of benzyl alcohol, or 8-10% by weight of coolant(s) in 92-90% by weight of benzyl alcohol, or
1-4% by weight coolant(s) in 99-96% by weight triethyl citrate, or
1-3% by weight coolant(s) in 99-97% by weight triacetin, or
3-6% by weight coolant(s) in 97-94% by weight diethyl succinate, or
5-15% by weight of coolant(s) in 95-85% by weight of 2-phenylethanol, or
5-10% by weight of coolant(s) in 95-90% by weight of benzyl benzoate, or
1-3% by weight coolant(s) in 99-97% by weight Optamint, or
1-4% by weight of coolant(s) in 99-96% by weight of other coolants as described above as component (b), or
2-4% by weight coolant(s) in 98-96% by weight propylene glycol, or
0.5-2% by weight coolant(s) in 95.5-98% by weight ethanol, or
0.5-2% by weight coolant(s) in 95.5-98% by weight menthyl acetate, or
1-4% by weight coolant(s) in 99-96% by weight peppermint oil, or
2-5 wt. % coolant(s) in 98-95 wt. % anethole,
   where both components (coolant(s) and solvent) in the coolant mixture always add up to 100% by weight. For example, a suitable coolant mixture according to the invention comprises 5-10 wt. % coolant(s) in 95-90 wt. % benzyl alcohol, particularly preferably 8-10 wt. % coolant(s) in 92-90 wt. % benzyl alcohol.

Another object of the present invention relates to a flavoring preparation comprising or consisting of
(d) one, two, three or more coolant(s) of general formulae (I) to (VIII) or as listed in Table 1 or Table 2 and defined above; and
(e) at least one flavoring agent.

The particular advantage of these mixtures or flavor preparations is that the coolants are able to mask unpleasant, for example bitter or astringent, taste impressions of flavors, especially of sweeteners, even in small concentrations, while at the same time providing an intensive and efficient cooling effect.

The above applies to component (d) in the same way as to component (a) of the physiological coolant mixture according to the invention, comprising or consisting of: (a) one, two, three or more coolant(s) of the general formulae (I) to (VIII), (Va), (VIa), (VIIa) or (VIIIa) or as listed in Table 1, Table 2, Table A or Table B and defined above

Die erfindungsgemiBen Aromazubereitungen umfassen einen oder mehrere Aromastoffe als Komponente (e), die ausgewshlt ist/sind aus der Gruppe, die gebildet wird von Acetophenon, Allylcapronat, alpha-lonon, beta-lonon, Anisaldehyd, Anisylacetat, Anisylformiat, Anethole, benzaldehyde, benzothiazole, benzyl acetate, benzyl alcohol, benzyl benzoate, beta-ionone, butyl butyrate, butyl capronate, butylidene phthalide, carvone, camphene, caryophyllene, cineole, cinnamyl acetate, citral, citronellol, citronellal, citronellyl acetate, cyclohexyl acetate, cymol, damascone, decalactone, dihydrocoumarin, dimethyl anthranilate, dimethyl anthranilate, dodecalactone, ethoxyethyl acetate, ethyl butyric acid, ethyl butyrate, ethyl caprinate, ethyl capronate, ethyl crotonate, ethyl furaneol, ethyl guaiacol, ethyl isobutyrate, ethyl isovalerate, ethyl lactate, ethyl methyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruit aldehyde, methyl dihydrojasmonate (e. g. B. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerate, isobutyl butyrate, isobutyraldehyde, isoeugenol methyl ether, isopropyl methyl thiazole, lauric acid, leavulinic acid, linalool, linalool oxide, linalyl acetate, menthol, menthofuran, methyl anthranilate, methyl butanol, methyl butyric acid, 2-methyl butyl acetate, methyl capronate, methyl cinnamate, 5-methyl furfural, 3,2,2-methyl cyclopentenolone, 6,5,2-methyl heptenone, methyl dihydrojasmonate, methyl jasmonate, 2-methyl methyl butyrate, 2-methyl-2-pentenolic acid, methyl thiobutyrate, 3,1-methyl thiohexanol, 3-methyl thiohexyl acetate, nerol, neryl acetate, trans,trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, piperonal, propionaldehyde, propyl butyrate, pulegone, pulegol, sinensal, sulfurol, terpinene, terpineol, terpinolene, 8,3-thiomenthanone, 4,4,2-thiomethylpentanone, thymol, delta-undecalactone, gamma-undecalactone, valencene, valeric acid, vanillic acid, vanillin, acetoin, ethyl vanillin, ethyl vanillin isobutyrate (=3-ethoxy-4-isobutyryloxybenzaldehyde), 2,5-dimethyl-4-hydroxy-3(2H)-furanone and its derivatives (preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (preferably ethylmaltol), coumarin and coumarin derivatives, gamma-lactones (preferably gamma-undecalactone, gamma-nonalactone, gamma-decalactone), delta-lactones (preferably 4-methyldeltadecalactone, massoilactone, deltadecalactone, tuberolactone), methyl sorbate, divanillin, 4-hydroxy-2(or 5)-ethyl-5(or 2)-methyl-3(2H)-furanone, 2-hydroxy-3-methyl-2-cyclopentenone, 3-hydroxy-4,5-dimethyl-2(5H)-furanone, acetic acid isoamyl ester, butyric acid ethyl ester, butyric acid n-butyl ester, butyric acid isoamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl 3-methyl-3-phenylglycidate, ethyl 2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furan, 2-methyl-3-furanethiol, bis(2-methyl-3-furyl)disulfide, furfuryl mercaptan, methional, 2-acetyl-2-thiazoline, 3-mercapto-2-pentanone, 2,5-dimethyl-3-furanethiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guaiacol, 3-hydroxy-4,5-dimethyl-2(5H)-furanone, 3-hydroxy-4-methyl-5-ethyl-2(5H)-furanone, cinnamaldehyde, cinnamic alcohol, methyl salicylate, isopulegol as well as stereoisomers, enantiomers, positional isomers, diastereomers, cis/trans isomers or epimers of these substances (not explicitly mentioned here). epimers of these substances.

For the purposes of the present invention, artificial as well as natural sweeteners and sweetener enhancers may also be considered as flavoring agents of component (e). These may be selected from the group consisting of
Sugar alcohols (e.g. erythritol, threitol, arabitol, ribotol, xylitol, sorbitol, mannitol, dulcitol, lactitol);
proteins (e.g. miraculin, monellin, thaumatin, curculin, brazzein);
Synthetic sweeteners (e.g. Magap, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatine, phenylodulcin);
Sweet-tasting amino acids (e.g. glycine, D-leucine, D-threonine, D-asparagine, D-phenylalanine, D-tryptophn, L-proline);
Sweet-tasting low molecular weight substances such as hernandulcin, dihydrochalcone glycosides, glycyrrhizin, glycerrhetinic acid, its derivatives and salts, extracts of licorice (Glycyrrhizza glabra ssp.), Lippia dulcis extracts, Momordica ssp. extracts and/or
Plant extracts, such as Momordica grosvenori [Luo Han Guo] and the mogrosides obtained from it, Hydrangea dulcis or Stevia ssp. (e.g. Stevia rebaudiana) extracts or steviosides obtained from it.

Component (e) comprises at least one of the above-mentioned flavoring agents.

The aroma preparations according to the invention may contain the components (d) and (e) in a weight ratio of about 1:99 to about 99:1, preferably about 10:90 to about 90 10, more preferably about 25:75 to about 75:25 and in particular about 40:60 to 60:40.

In a further preferred variant, the one or more coolant(s) or the coolant mixture or the aroma preparation is present in encapsulated form. This is of particular interest, for example, if the capsules loaded with the one or more coolant(s) are applied to textile surfaces, for example as a component of fabric softeners or laundry after-treatment agents, or if finishing is carried out by using capsules loaded with the one or more coolant(s) by forced application, for example on tights.

Capsules are spherical aggregates containing at least one solid or liquid core enclosed in at least one continuous shell. During encapsulation, the one or more coolant(s), or the coolant mixture or the flavor preparation is encapsulated with the aid of a coating material/envelope material, so that these are in the form of macrocapsules with diameters of about 0.1 to about 5 mm or microcapsules with diameters of about 0.0001 to about 0.1 mm.

Consequently, a further embodiment of the present invention also relates to physiological coolants or physiological coolant mixtures or flavoring preparations in encapsulated form.

Suitable coating materials are, for example, starches, including their degradation products and chemically or physically produced derivatives (in particular dextrins and maltodextrins), gelatine, gum arabic, agar-agar, ghatti gum, gellan gum, modified and unmodified celluloses, pullulan, curdlan, carrageenans, alginic acid, alginates, pectin, inulin, xanthan gum and mixtures of two or more of these substances.

Among the above-mentioned coating materials, gelatine (in particular porcine, bovine, poultry and/or fish gelatine) is preferred, preferably having a swelling factor of greater than or equal to 20, preferably greater than or equal to 24. Gelatine is also particularly preferred as it is readily available and can be obtained with different swelling factors.

Also preferred are maltodextrins (in particular based on cereals, especially maize, wheat, tapioca or potatoes), which preferably have DE values in the range from 10 to 20. Celluloses (e.g. cellulose ether), alginates (e.g. sodium alginate), carrageenan (e.g. beta-, iota-, lambda- and/or kappa-carrageenan), gum arabic, curdlan and/or agar agar are also preferred.

Also preferred are alginate capsules, such as those described in detail in the following publications: EP 0389700 A1, U.S. Pat. Nos. 4,251,195, 6,214,376, WO 2003 055587 or WO 2004 050069 A1.

In a further preferred embodiment, the shell of the capsules consists of melamine-formaldehyde resins or coacervation products of cationic monomers or biopolymers (such as chitosan) and anionic monomers, such as (meth)acrylates or alginates.

The capsules are generally finely dispersed liquid or solid phases coated with film-forming polymers, during the production of which the polymers are deposited on the material to be coated after emulsification and coacervation or interfacial polymerization. According to another method, melted waxes are incorporated in a matrix (“microsponge”), which can also be coated as microparticles with film-forming polymers. According to a third method, particles are alternately coated with polyelectrolytes of different charges (“layer-by-layer” method). The microscopically small capsules can be dried and used like powder.

In addition to mononuclear microcapsules, multinuclear aggregates, also known as microspheres, are also known, which contain two or more nuclei distributed in the continuous shell material. Mononuclear or multinuclear microcapsules can also be enclosed by an additional second, third etc. shell. The shell can consist of natural, semi-synthetic or synthetic materials. Natural shell materials include gum arabic, agar-agar, agarose, maltodextrins, alginic acid or its salts, e.g. sodium or calcium alginate, fats and fatty acids, cetyl alcohol, collagen, chitosan, lecithin, gelatine, albumin, shellac, polysaccharides such as starch or dextran, polypeptides, protein hydrolysates, sucrose and waxes. Semi-synthetic coating materials include chemically modified celluloses, in particular cellulose esters and -ethers, e.g. cellulose acetate, ethyl cellulose, hydroxypropyl cellulose, hydroxypropyl methyl cellulose and carboxymethyl cellulose, as well as starch derivatives, in particular starch ethers and -esters. Synthetic coating materials are, for example, polymers such as polyacrylates, polyamides, polyvinyl alcohol or polyvinylpyrrolidone.

Examples of coating materials/shell materials of the prior art for the production of microcapsules are the following commercial products (the shell material is indicated in brackets): Hal/crest Microcapsules (gelatin, gum arabic), Coletica Thalaspheres (marine collagen), Lipotec Millicapsules (alginic acid, agar-agar), Induchem Unispheres (lactose, microcrystalline cellulose, hydroxypropylmethylcellulose), Unicerin C30 (lactose, microcrystalline cellulose, hydroxypropylmethylcellulose), Kobo Glycospheres (modified starch, fatty acid esters, phospholipids), Softspheres (modified agar-agar) and Kuhs Probiol Nanospheres (phospholipids) as well as Primaspheres and Primasponges (chitosan, alginates) and Primasys (phospholipids).

Chitosan microcapsules and methods for their production are well known from the state of the art: WO 01/01926, WO 01/01927, WO 01/01928, WO 01/01929. Microcapsules with mean diameters in the range from 0.0001 mm to 5 mm, preferably 0.001 mm to 0.5 mm and in particular 0.005 mm to 0.1 mm, consisting of a shell membrane and a matrix containing the active ingredients, can be obtained, for example, by
(1) a matrix is prepared from gel formers, cationic polymers and active ingredients,
(2) if necessary, the matrix is dispersed in an oil phase,
(3) the dispersed matrix is treated with aqueous solutions of anionic polymers and, if necessary, the oil phase is removed.

The aforementioned steps (1) and (3) are interchangeable insofar as anionic polymers are used instead of the cationic polymers in step (1) and vice versa.

The capsules can also be produced by alternately coating the active ingredient with layers of differently charged polyelectrolytes (layer-by-layer technology). In this context, reference is made to the European patent EP 1064088 B1 (Max Planck Society).

The two essential properties of the new coolants or new coolant mixtures are, as already mentioned, on the one hand to modulate the TRPM8 receptor as antagonists or agonists and in this way trigger a physiological reaction, namely an intensive and efficient cooling effect on the skin or mucous membrane, and on the other hand to reduce or mask unpleasant flavors. Primarily, however, the ability to produce intensive and efficient cooling effects even in small quantities should be emphasized.

A further aspect of the present invention therefore relates to the use of the physiological coolant according to the invention or the physiological coolant mixture according to the invention as a modulator, preferably for in vivo and/or in vitro modulation, of the cold menthol receptor TRPM8, in particular as a TRPM8 receptor agonist or as a TRPM8 receptor antagonist.

In the use according to the invention, the receptor TRPM8 is brought into contact with at least one coolant according to the invention or a physiological coolant mixture according to the invention, which modulates the permeability of these cells for Ca²⁺ ions in a cellular activity test using cells which recombinantly express the human TRPM8 receptor.

Suitable modulators can act either only as antagonists or agonists, in particular only as agonists, or both as antagonists and agonists. In particular, an agonistic or an antagonistic effect can occur depending on the respective modulator concentration selected.

An “agonist” is a substance that mediates activation of the TRPM8 receptor, i.e. induces an influx of Ca²⁺ ions into the cold-sensitive neurons and thus conveys a sensation of cold.

An “antagonist”, on the other hand, is a compound that can counteract this activation of the TRPM8 receptor.

The modulators according to the invention, i.e. the one physiological coolant or the coolant mixture, can exert their effect by binding reversibly or irreversibly, specifically or non-specifically to a TRPM8 receptor molecule. Binding is usually non-covalent via ionic and/or non-ionic, e.g. hydrophobic, interactions with the receptor molecule. The term “specific” includes both exclusive interaction with one or more different TRPM8 receptor molecules (such as TRPM8 molecules of different origin or different isoforms). The term “non-specific”, on the other hand, is an interaction of the modulator with several different receptor molecules of different function and/or sequence, whereby, however, a desired agonistic and/or antagonistic modulation (as described above) of the TRPM8 receptor can be determined as a result.

In a use according to the invention, preferably in a variant described above as preferred, the modulator has an agonistic or antagonistic effect on the cellular Ca²⁺ ion permeability.

A variant of the use according to the invention in which the modulator is a TRPM8 receptor agonist is particularly preferred.

Due to its physiological property of inducing a cooling effect on the skin or mucous membrane, a further aspect of the present invention relates to the use of the coolant or coolant mixture according to the invention for producing a physiological cooling effect on the skin or mucous membrane of a human or animal.

Alternatively, the coolant according to the invention or the coolant mixture according to the invention is used to induce a cooling effect by means of a package containing the physiological coolant or the physiological coolant mixture or a textile containing the physiological coolant or the physiological coolant mixture.

Due to its additional property(ies) of reducing or masking unpleasant, for example bitter or astringent, flavors, a further aspect of the present invention relates to the use of the physiological coolant or the coolant mixture according to the invention to improve the taste properties of flavorings. This allows known taste disadvantages of flavorings, especially of sweeteners such as steviosides, to be reduced or masked. In particular, the pungent, bitter or metallic aftertaste is effectively reduced or masked even when small quantities are added.

The coolants according to the invention or the physiological coolant mixtures according to the invention or the aroma preparations according to the invention have a broad field of application, in particular in foodstuffs, in food supplements, cosmetic or pharmaceutical preparations, animal feed, textiles, packaging or tobacco products.

In particular, the physiological coolants or the physiological coolant mixtures or the flavoring preparations according to the invention are used for the production of foodstuffs, food supplements, cosmetic or pharmaceutical preparations, animal feed, textiles, packaging or tobacco products due to their cooling properties and/or flavor-enhancing properties.

A further object of the present invention is therefore the use of one or more coolants according to the invention or the coolant mixture according to the invention or the flavoring preparation according to the invention for the manufacture of foodstuffs, food supplements, cosmetic or pharmaceutical preparations, animal feed, textiles, packaging or tobacco products.

Due to the advantageous properties described, the coolants according to the invention, as represented and defined by the general formulae (I) to (VIII), (Va), (VIa), (VIIa) or (Villa) or as shown in Table 1, Table 2, Table A or Table B, are suitable for the uses according to the invention, namely use as a modulator, for producing a physiological cooling effect on the skin or mucous membrane of humans or animals or for inducing a cooling effect, for improving the taste properties of flavoring substances, in particular for reducing or masking an unpleasant taste, for the manufacture of foodstuffs, food supplements, cosmetic or pharmaceutical preparations, animal feedstuffs, textiles, packaging or tobacco products, or for use as a medicament, as described in detail herein, preferably selected from the group consisting of the compounds shown in Table 5 or in Table 6.

TABLE 5
Preferred structures according to the invention

| | TRPM8 | TRPM8 | Sensory | | |
|---|---|---|---|---|---|
| Compound | activation | EC50 [μM] | evaluation | Structure | IUPAC name |
| B-01 | 262 | 0.05 | 4.2 | 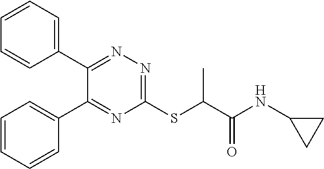 [SMILES: CC(Sc1nnc(-c2ccccc2)c(-c2ccccc2)n1)C(=O)NC1CC1] | N-Cyclopropyl-2- [(5,6-diphenyl- 1,2,4-triazin-3-yl)sulfanyl] propanamide |
| B-02 | 227 | 0.08 | 4.1 | 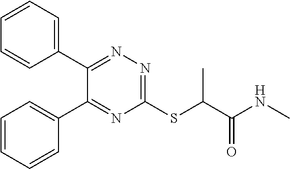 [SMILES: CNC(=O)C(C)Sc1nnc(-c2ccccc2)c(-c2ccccc2)n1] | 2-(5,6-diphenylpyrazin- 2-yl)sulfanyl-N- methyl-propanamide |
| B-03 | 221 | 0.07 | | 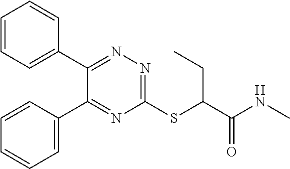 [SMILES: CCC(Sc1nnc(-c2ccccc2)c(-c2ccccc2)n1)C(=O)NC] | 2-[(5,6-diphenyl- 1,2,4-triazin-3- yl)sulfanyl]-N- methyl-butanamide |
| B-04 | 210 | 0.37 | | 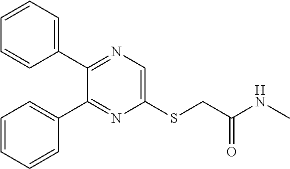 [SMILES: CNC(=O)CSc1cnc(-c2ccccc2)c(-c2ccccc2)n1] | 2-(5,6-diphenylpyrazin- 2-yl)sulfanyl-N- methyl-acetamide |
| B-05 | 205 | 0.1 | | 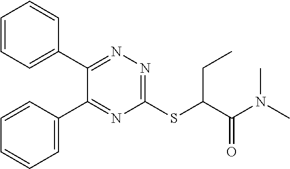 [SMILES: CCC(Sc1nnc(-c2ccccc2)c(-c2ccccc2)n1)C(=O)N(C)C] | 2-[(5,6-diphenyl- 1,2,4-triazin-3- yl)sulfanyl]-N,N- dimethyl-butanamide |
| B-06 | 205 | 0.28 | 3.0 | 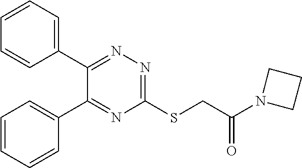 [SMILES: O=C(CSc1nnc(-c2ccccc2)c(-c2ccccc2)n1)N1CCC1] | 1-(azetidin-1-yl)- 2-[(5,6-diphenyl- 1,2,4-triazin-3- yl)sulfanyl]ethanone |
| B-07 | 203 | 0.92 | 0.75 | 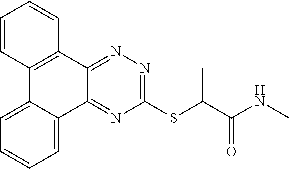 [SMILES: CNC(=O)C(C)Sc1nnc2c3ccccc3c3ccccc3c2n1] | N-methyl-2- phenanthro[9,10- e][1,2,4]triazine- 3-ylsulfanyl-propanamide |
| B-11 | 143 | 1.43 | 5.3 | 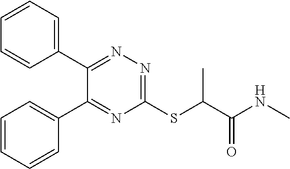 [SMILES: CNC(=O)C(C)Sc1nnc(-c2ccccc2)c(-c2ccccc2)n1] | 2-[(5,6-diphenyl- 1,2,4-triazin-3- yl)sulfanyl]-N- methyl-propanamide |
| B-14 | 122 | | | 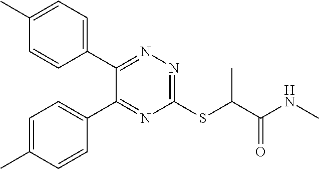 | 2-[[5,6-bis(p- tolyl)-1,2,4-triazin- 3-yl]sulfanyl]-N- methyl-propanamide |
| B-15 | 109 | | | 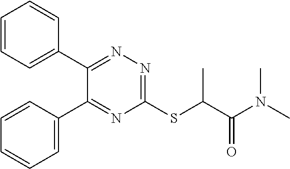 [SMILES: CC(Sc1nnc(-c2ccccc2)c(-c2ccccc2)n1)C(=O)N(C)C] | 2-[(5,6-diphenyl- 1,2,4-triazin-3- yl)sulfanyl]-N,N- dimethyl-propanamide |
| B-17 | 102 | | | 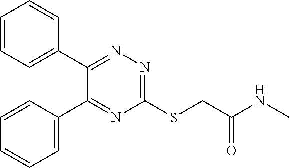 [SMILES: CNC(=O)CSc1nnc(-c2ccccc2)c(-c2ccccc2)n1] | 2-[(5,6-diphenyl- 1,2,4-triazin-3- yl)sulfanyl]-N- methyl-acetamide |
| B-18 | 101 | 5.36 | | 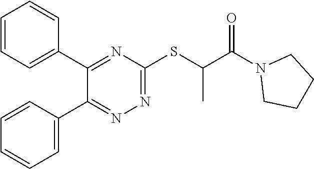 [SMILES: CC(Sc1nnc(-c2ccccc2)c(-c2ccccc2)n1)C(=O)N1CCCC1] | 2-[(5,6-diphenyl- 1,2,4-triazin-3- yl)sulfanyl]-1- pyrrolidin-1-yl- propan-1-one |
| B-19 | 101 | | 1.9 | 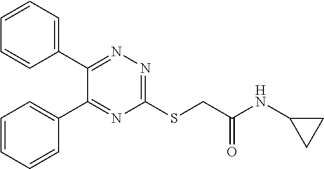 [SMILES: O=C(CSc1nnc(-c2ccccc2)c(-c2ccccc2)n1)NC1CC1] | N-cyclopropyl-2- [(5,6-diphenyl- 1,2,4-triazin-3- yl)sulfanyl]acetamide |
| B-21 | 90 | | | 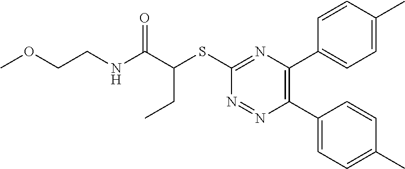 | 2-[[5,6-bis (p-tolyl)-1,2,4-triazin- 3-yl]sulfanyl]-N- (2-methoxyethyl) butanamide |
| B-22 | 87 | | | 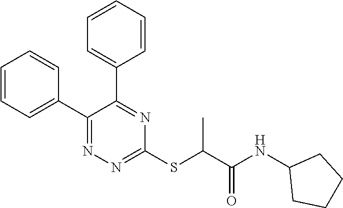 [SMILES: CC(Sc1nnc(-c2ccccc2)c(-c2ccccc2)n1)C(=O)NC1CCCC1] | N-cyclopentyl-2- [(5,6-diphenyl- 1,2,4-triazin-3- yl)sulfanyl]propanamide |
| B-23 | 73 | | | 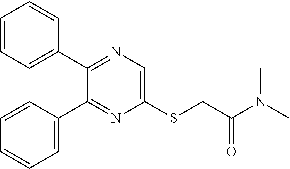 [SMILES: CN(C)C(=O)CSc1cnc(-c2ccccc2)c(-c2ccccc2)n1] | 2-(5,6- diphenylpyrazin- 2-yl)sulfanyl-N,N- dimethyl-acetamide |
| B-24 | 68 | | | 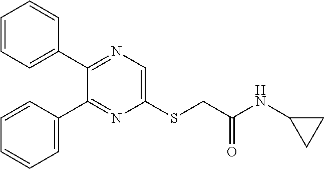 [SMILES: O=C(CSc1cnc(-c2ccccc2)c(-c2ccccc2)n1)NC1CC1] | N-cyclopropyl-2- (5,6- diphenylpyrazin- 2-yl)sulfanyl-acetamide |
| B-25 | 57 | | | 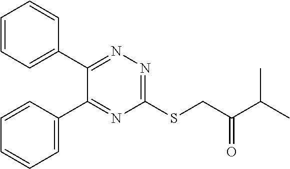 [SMILES: CC(C)C(=O)CSc1nnc(-c2ccccc2)c(-c2ccccc2)n1] | 1-[(5,6-diphenyl- 1,2,4-triazin-3- yl)sulfanyl]-3- methyl-butan-2-one |
| B-26 | 56 | | | 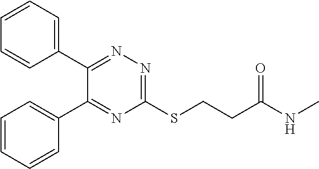 [SMILES: CNC(=O)CCSc1nnc(-c2ccccc2)c(-c2ccccc2)n1] | 3-[(5,6-diphenyl- 1,2,4-triazin-3- yl)sulfanyl]-N- methyl-propanamide |
| B-27 | 56 | | | 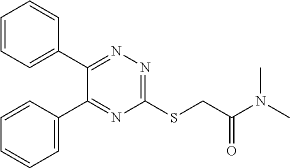 [SMILES: CN(C)C(=O)CSc1nnc(-c2ccccc2)c(-c2ccccc2)n1] | 2-[(5,6-diphenyl- 1,2,4-triazin-3- yl)sulfanyl]-N,N- dimethyl-acetamide |
| B-28 | 53 | | | 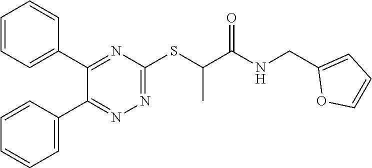 [SMILES: CC(Sc1nnc(-c2ccccc2)c(-c2ccccc2)n1)C(=O)NCc1ccco1] | 2-[(5,6-diphenyl- 1,2,4-triazin-3- yl)sulfanyl]-N-(2- furylmethyl)propanamide |
| B-29 | 42 | | | 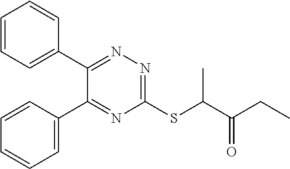 [SMILES: CCC(=O)C(C)Sc1nnc(-c2ccccc2)c(-c2ccccc2)n1] | 2-[(5,6-diphenyl- 1,2,4-triazin-3- yl)sulfanyl]pentan- 3-one |
| B-30 | 41 | | | 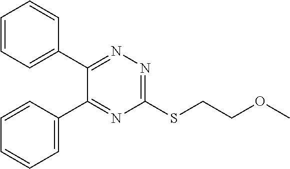 [SMILES: COCCSc1nnc(-c2ccccc2)c(-c2ccccc2)n1] | 3-(2- methoxyethyl- sulfanyl)-5,6-diphenyl- 1,2,4-triazine |
| B-31 | 40 | | | 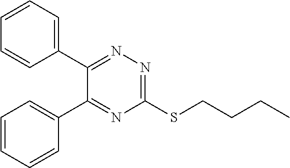 [SMILES: CCCCSc1nnc(-c2ccccc2)c(-c2ccccc2)n1] | 3-butylsulfanyl- 5,6-diphenyl- 1,2,4-triazine |
| B-32 | 37 | | | 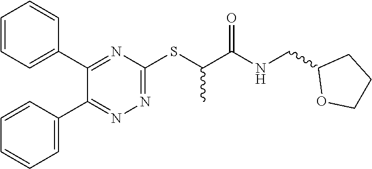 [SMILES: CC(Sc1nnc(-c2ccccc2)c(-c2ccccc2)n1)C(=O)NCC1CCCO1] | 2-[(5,6-diphenyl- 1,2,4-triazin-3- yl)sulfanyl]-N- (tetrahydrofuran- 2-ylmethyl)propanamide |
| B-33 | 25 | | | 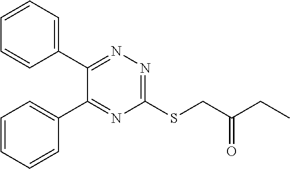 [SMILES: CCC(=O)CSc1nnc(-c2ccccc2)c(-c2ccccc2)n1] | 1-[(5,6-diphenyl- 1,2,4-triazin-3- yl)sulfanyl]butan-2-one |
| B-34 | 25 | | | 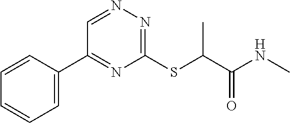 [SMILES: CNC(=O)C(C)Sc1nncc(-c2ccccc2)n1] | N-methyl-2-[(5- phenyl-1,2,4-triazin-3- yl)sulfanyl]propanamide |
| B-36 | 20 | | | 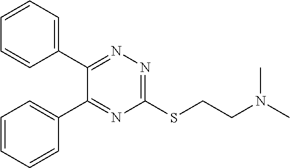 [SMILES: CN(C)CCSc1nnc(-c2ccccc2)c(-c2ccccc2)n1] | 2-[(5,6-diphenyl- 1,2,4-triazin-3- yl)sulfanyl]-N,N- dimethyl-ethanamine |
| B-37 | 13 | | | 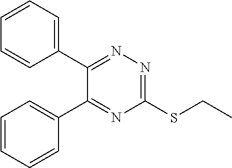 [SMILES: CCSc1nnc(-c2ccccc2)c(-c2ccccc2)n1] | 3-ethylsulfanyl- 5,6-diphenyl- 1,2,4-triazine |
| B-38 | 11 | | | 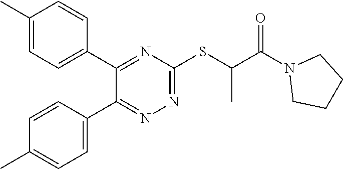 [SMILES: Cc1ccc(-c2nnc(SC(C)C(=O)N3CCCC3)nc2-c2ccc(C)cc2)cc1] | 2-[[5,6-bis(p- tolyl)-1,2,4-triazin- 3-yl]sulfanyl]-1- pyrrolidin-1-yl- propan-1-one |
| B-39 | 11 | | | 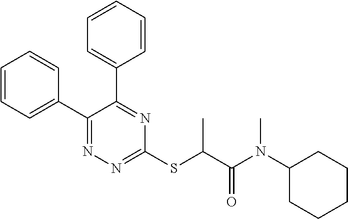 [SMILES: CC(Sc1nnc(-c2ccccc2)c(-c2ccccc2)n1)C(=O)N(C)C1CCCCC1] | N-cyclohexyl-2- [(5,6-diphenyl- 1,2,4-triazin-3- yl)sulfanyl]-N- methyl-propanamide |
| B-40 | 9 | | | 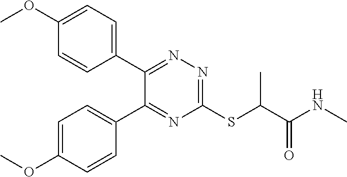 | 2-[[5,6-bis(4- methoxyphenyl)- 1,2,4-triazin-3- yl]sulfanyl]-N- methyl-propanamide |
| B-41 | 8 | | | 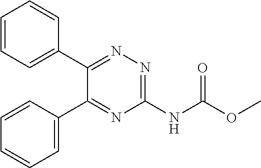 [SMILES: COC(=O)Nc1nnc(-c2ccccc2)c(-c2ccccc2)n1] | methyl N-(5,6- diphenyl-1,2,4- triazin-3-yl)carbamate |
| B-42 | 8 | | | 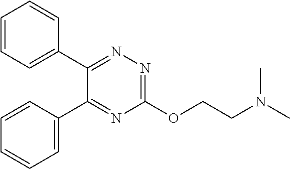 [SMILES: CN(C)CCOc1nnc(-c2ccccc2)c(-c2ccccc2)n1] | 2-[(5,6-diphenyl- 1,2,4-triazin-3- yl)oxy]-N,N- dimethyl-ethanamine |
| B-43 | 6 | | | 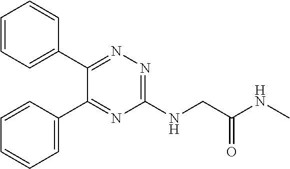 [SMILES: CNC(=O)CNc1nnc(-c2ccccc2)c(-c2ccccc2)n1] | 2-[(5,6-diphenyl- 1,2,4-triazin-3- yl)amino]-N- methyl-acetamide |
| B-44 | 5 | | | 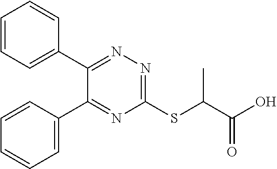 [SMILES: CC(Sc1nnc(-c2ccccc2)c(-c2ccccc2)n1)C(=O)O] | 2-[(5,6-diphenyl- 1,2,4-triazin-3- yl)sulfanyl]propanoic acid |
| B-45 | 5 | | | 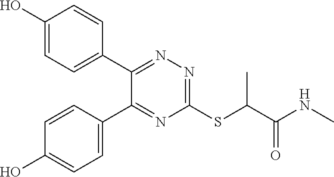 [SMILES: CNC(=O)C(C)Sc1nnc(-c2ccc(O)cc2)c(-c2ccc(O)cc2)n1] | 2-[[5,6-bis(4- hydroxyphenyl)- 1,2,4-triazin-3- yl]sulfanyl]-N- methyl-propanamide |
| B-46 | 5 | | | 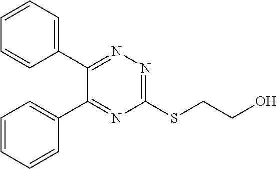 [SMILES: OCCSc1nnc(-c2ccccc2)c(-c2ccccc2)n1] | 2-[(5,6-diphenyl- 1,2,4-triazin-3- yl)sulfanyl]ethanol |
| B-47 | 4 | | | 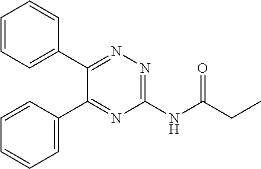 [SMILES: CCC(=O)Nc1nnc(-c2ccccc2)c(-c2ccccc2)n1] | N-(5,6-diphenyl- 1,2,4-triazin-3- yl)propanamide |
| B-48 | 4 | | | 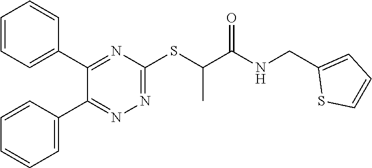 [SMILES: CC(Sc1nnc(-c2ccccc2)c(-c2ccccc2)n1)C(=O)NCc1cccs1] | 2-[(5,6-diphenyl- 1,2,4-triazin-3- yl)sulfanyl]-N-(2- thienylmethyl) propanamide |
| B-49 | 3 | | | 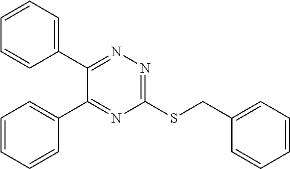 [SMILES: c1ccc(CSc2nnc(-c3ccccc3)c(-c3ccccc3)n2)cc1] | 3-benzylsulfanyl- 5,6-diphenyl- 1,2,4-triazine |
| B-50 | 3 | | | 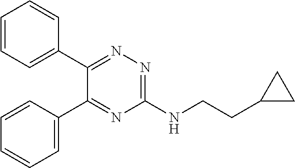 [SMILES: c1ccc(-c2nnc(NCCC3CC3)nc2-c2ccccc2)cc1] | N-(2- cyclopropylethyl)- 5,6-diphenyl- 1,2,4-triazin-3-amine |
| B-51 | 3 | | | 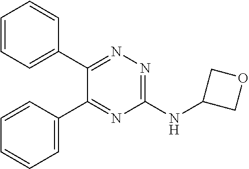 [SMILES: c1ccc(-c2nnc(NC3COC3)nc2-c2ccccc2)cc1] | N-(oxetan-3-yl)- 5,6-diphenyl- 1,2,4-triazin-3-amine |
| B-52 | 3 | | | 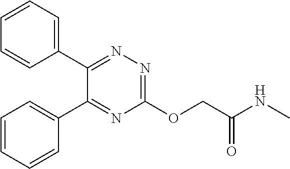 [SMILES: CNC(=O)COc1nnc(-c2ccccc2)c(-c2ccccc2)n1] | 2-[(5,6-diphenyl- 1,2,4-triazin-3- yl)oxy]-N-methyl- acetamide |
| B-53 | 2 | | | 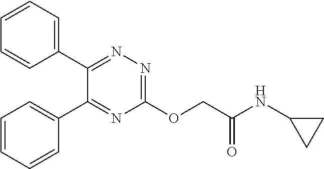 [SMILES: O=C(COc1nnc(-c2ccccc2)c(-c2ccccc2)n1)NC1CC1] | N-cyclopropyl-2- [(5,6-diphenyl- 1,2,4-triazin-3- yl)oxy]acetamide |
| B-54 | 2 | | | 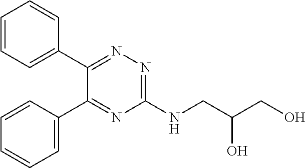 [SMILES: OCC(O)CNc1nnc(-c2ccccc2)c(-c2ccccc2)n1] | 3-[(5,6-diphenyl- 1,2,4-triazin-3- yl)amino]propane-1,2-diol |
| B-55 | 2 | | | 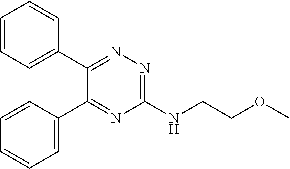 [SMILES: COCCNc1nnc(-c2ccccc2)c(-c2ccccc2)n1] | N-(2- methoxyethyl)- 5,6-diphenyl- 1,2,4-triazin-3-amine |
| B-56 | 2 | | | 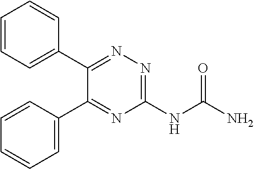 [SMILES: NC(=O)Nc1nnc(-c2ccccc2)c(-c2ccccc2)n1] | (5,6-diphenyl- 1,2,4-triazin-3-yl)urea |
| B-57 | 2 | | | 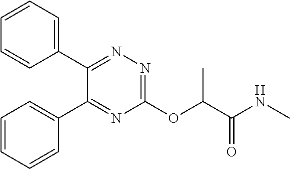 [SMILES: CNC(=O)C(C)Oc1nnc(-c2ccccc2)c(-c2ccccc2)n1] | 2-[(5,6-diphenyl- 1,2,4-triazin-3- yl)oxy]-N-methyl- propanamide |
| B-58 | 1 | | | 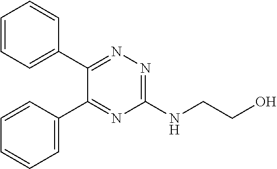 [SMILES: OCCNc1nnc(-c2ccccc2)c(-c2ccccc2)n1] | 2-[(5,6-diphenyl- 1,2,4-triazin-3- yl)amino]ethanol |
| B-59 | 1 | | | 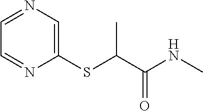 | N-methyl-2- pyrazine-2- ylsulfanyl- propanamide |
| B-60 | 1 | | | 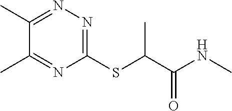 | 2-[(5,6-dimethyl- 1,2,4-triazin-3- yl)sulfanyl]-N- methyl-propanamide |
| B-61 | 1 | | | 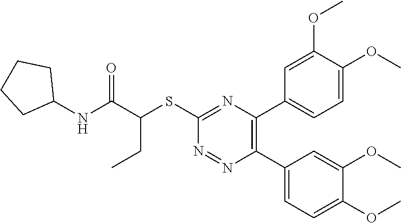 [SMILES: CCC(Sc1nnc(-c2ccc(OC)c(OC)c2)c(-c2ccc(OC)c(OC)c2)n1)C(=O)NC1CCCC1] | 2-[[5,6-bis(3,4- dimethoxyphenyl)- 1,2,4-triazin-3- yl]sulfanyl]-N- cyclopentyl-butanamide |
| B-62 | 0 | | | 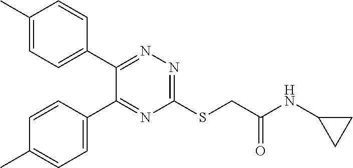 [SMILES: Cc1ccc(-c2nnc(SCC(=O)NC3CC3)nc2-c2ccc(C)cc2)cc1] | 2-[[5,6-bis(p- tolyl)-1,2,4-triazin- 3-yl]sulfanyl]-N- cyclopropyl-acetamide |
| B-63 | −2 | | | 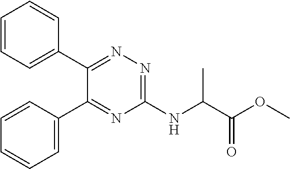 [SMILES: COC(=O)C(C)Nc1nnc(-c2ccccc2)c(-c2ccccc2)n1] | methyl 2-[(5,6- diphenyl-1,2,4-triazin-3- yl)amino]propanoate |
| B-64 | −3 | | | 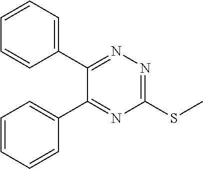 [SMILES: CSc1nnc(-c2ccccc2)c(-c2ccccc2)n1] | 3-methylsulfanyl- 5,6-diphenyl- 1,2,4-triazine |
| B-65 | | | | 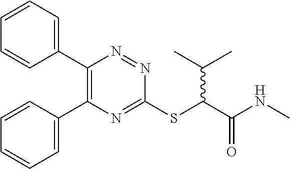 [SMILES: CNC(=O)C(Sc1nnc(-c2ccccc2)c(-c2ccccc2)n1)C(C)C] | 2-[(5,6-diphenyl- 1,2,4-triazin-3- yl)sulfanyl]-N,3-dimethyl- butanamide |
| B-66 | | | | 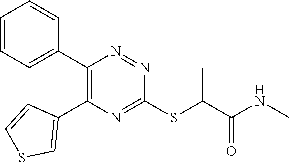 [SMILES: CNC(=O)C(C)Sc1nnc(-c2ccccc2)c(-c2ccsc2)n1] | N-methyl-2-[[6- phenyl-5-(3- thienyl)-1,2,4- triazin-3- yl]sulfanyl]propanamide |
| B-67 | | | | 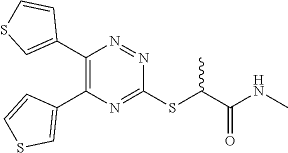 [SMILES: CNC(=O)C(C)Sc1nnc(-c2ccsc2)c(-c2ccsc2)n1] | 2-[[5,6-bis(3- thienyl)-1,2,4-triazin-3- yl]sulfanyl]-N-methyl- propanamide |
| B-68 | | | | 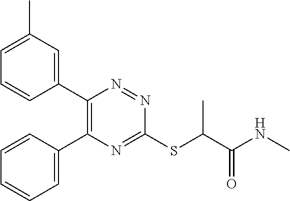 [SMILES: CNC(=O)C(C)Sc1nnc(-c2cccc(C)c2)c(-c2ccccc2)n1] | N-methyl-2-[[6- (m-tolyl)-5- phenyl-1,2,4- triazin-3- yl]sulfanyl]propanamide |
| B-69 | | | | 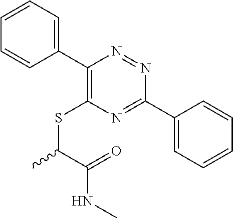 [SMILES: CNC(=O)C(C)Sc1nc(-c2ccccc2)nnc1-c1ccccc1] | 2-[(3,6-diphenyl- 1,2,4-triazin-5- yl)sulfanyl]-N- methyl-propanamide |
| B-70 | | | | 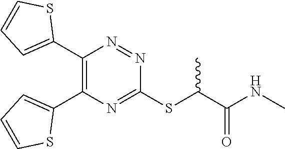 [SMILES: CNC(=O)C(C)Sc1nnc(-c2cccs2)c(-c2cccs2)n1] | 2-[[5,6-bis(2- thienyl)-1,2,4-triazin-3- yl]sulfanyl]-N-methyl- propanamide |
| B-71 | | | | 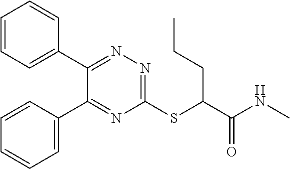 [SMILES: CCCC(Sc1nnc(-c2ccccc2)c(-c2ccccc2)n1)C(=O)NC] | 2-[(5,6-diphenyl- 1,2,4-triazin-3- yl)sulfanyl]-N- methyl- pentanamide |
| B-72 | | | | 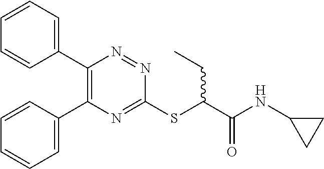 [SMILES: CCC(Sc1nnc(-c2ccccc2)c(-c2ccccc2)n1)C(=O)NC1CC1] | N-cyclopropyl-2- [(5,6-diphenyl- 1,2,4-triazin-3- yl)sulfanyl]butanamide |
| B-73 | | | | 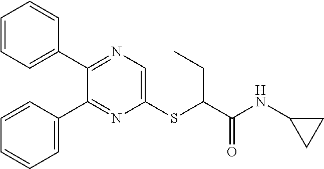 [SMILES: CCC(Sc1cnc(-c2ccccc2)c(-c2ccccc2)n1)C(=O)NC1CC1] | N-cyclopropyl-2-(5,6- diphenylpyrazin- 2-yl)sulfanyl- butanamide |
| B-74 | | | | 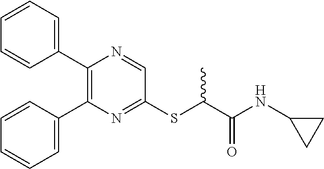 [SMILES: CC(Sc1cnc(-c2ccccc2)c(-c2ccccc2)n1)C(=O)NC1CC1] | N-cyclopropyl-2-(5,6- diphenylpyrazin- 2-yl)sulfanyl- propanamide |
| B-75 | | | | 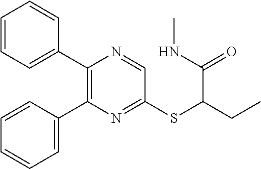 [SMILES: CCC(Sc1cnc(-c2ccccc2)c(-c2ccccc2)n1)C(=O)NC] | 2-[(5,6- Diphenylpyrazin- 2-yl)sulfanyl]-N- methylbutanamide |
| B-76 | | | | 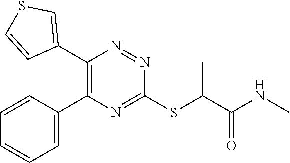 [SMILES: CNC(=O)C(C)Sc1nnc(-c2ccsc2)c(-c2ccccc2)n1] | N-methyl-2-[[5- phenyl-6-(3- thienyl)-1,2,4-triazin-3- yl]sulfanyl]propanamide |
| B-77 | | | | 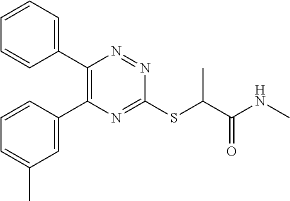 [SMILES: CNC(=O)C(C)Sc1nnc(-c2ccccc2)c(-c2cccc(C)c2)n1] | N-methyl-2-[[5- (m-tolyl)-6- phenyl-1,2,4-triazin-3- yl]sulfanyl]propanamide |

TABLE 6
Preferably used structures according to the invention

| | | TRPM8 | | | |
|---|---|---|---|---|---|
| Com- | TRPM8 | EC50 | Sensory | | |
| pound | activation | [μM] | evaluation | Structure | IUPAC name |
| A-01 | 278 | 0.01 | 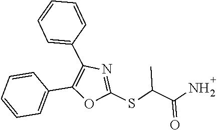 [SMILES: CNC(=O)C(C)Sc1nc(-c2ccccc2)c(-c2ccccc2)o1] | 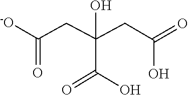 | 3-carboxy-3,5- dihydroxy-5- OXO- pentanoate;2- (4,5- diphenyloxazol- 2- yl)sulfanylpropa noyl-methyl- ammonium |
| A-02 | 265 | 0.01 | 5.4 | 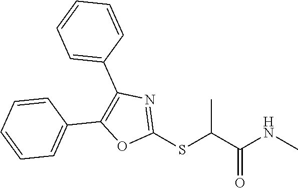 [SMILES: CNC(=O)C(C)Sc1nc(-c2ccccc2)c(-c2ccccc2)o1] | 2-(4,5- diphenyloxazol- 2-yl)sulfanyl-N- methyl- propanamide |
| A-03 | 260 | 0.01 | | 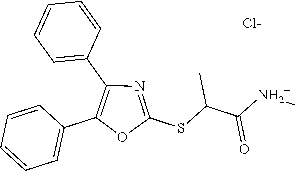 [SMILES: C[NH2+]C(=O)C(C)Sc1nc(-c2ccccc2)c(-c2ccccc2)o1 ; [Cl-]] | 2-(4,5- diphenyloxazol- 2- yl)sulfanylpropa noyl-methyl- ammonium; chloride |
| A-04 | 215 | 0.6 | 0.5 | 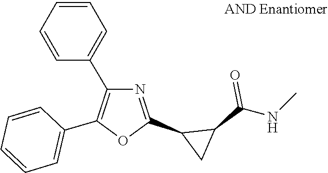 [SMILES: CNC(=O)[C@H]1C[C@H]1c1nc(-c2ccccc2)c(-c2ccccc2)o1] | rac-(1S,2R)-2- (4,5- diphenyloxazol- 2-yl)-N-methyl- cyclopropane- carboxamides |
| A-05 | 190 | 2.31 | 1.1 | 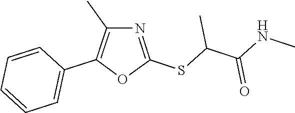 [SMILES: CNC(=O)C(C)Sc1nc(C)c(-c2ccccc2)o1] | N-methyl-2-(4- methyl-5- phenyl-oxazol- 2-yl)sulfanyl- propanamide |
| A-06 | 186 | 0.34 | | 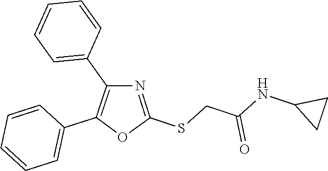 [SMILES: O=C(CSc1nc(-c2ccccc2)c(-c2ccccc2)o1)NC1CC1] | N-cyclopropyl-2- (4,5- diphenyloxazol- 2-yl)sulfanyl- acetamide |
| A-07 | 183 | 0.15 | 2.67 | 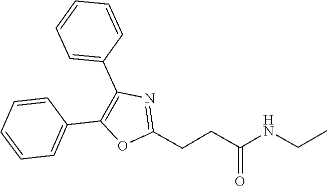 [SMILES: CCNC(=O)CCc1nc(-c2ccccc2)c(-c2ccccc2)o1] | 3-(4,5- diphenyloxazol- 2-yl)-N-ethyl- propanamide |
| A-08 | 177 | 0.11 | | 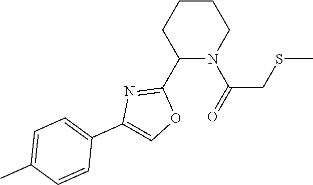 | 2- methylsulfanyl- 1-[2-[4-(p- tolyl)oxazol-2- yl]-1- piperidyl] ethanone |
| A-09 | 120 | 1.54 | 4.66 | 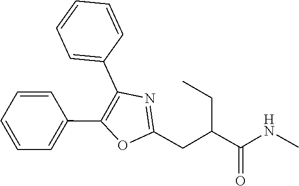 [SMILES: CCC(Cc1nc(-c2ccccc2)c(-c2ccccc2)o1)C(=O)NC] | 2-(4,5- diphenyloxazol- 2-yl)sulfanyl-N- methyl- butanamide |
| A-10 | 117 | 2.13 | 5.38 | 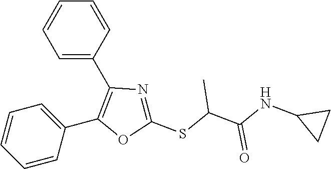 [SMILES: CC(Sc1nc(-c2ccccc2)c(-c2ccccc2)o1)C(=O)NC1CC1] | N-cyclopropyl-2- (4,5- diphenyloxazol- 2-yl) sulfanyl- propanamide |
| A-11 | 5 | | | 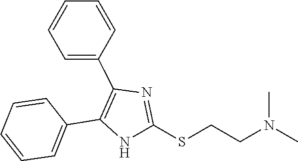 [SMILES: CN(C)CCSc1nc(-c2ccccc2)c(-c2ccccc2)[nH]1] | 2-[(4,5-diphenyl- 1H-imidazol-2- yl)sulfanyl]-N,N- dimethyl- ethanamine |
| A-12 | 112 | 5.09 | 1.26 | 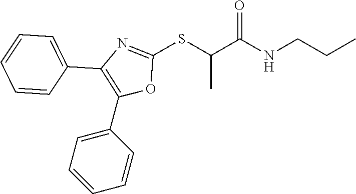 [SMILES: CCCNC(=O)C(C)Sc1nc(-c2ccccc2)c(-c2ccccc2)o1] | 2-(4,5- diphenyloxazol- 2-yl)sulfanyl-N- propyl- propanamide |
| A-15 | 98 | 7.14 | | 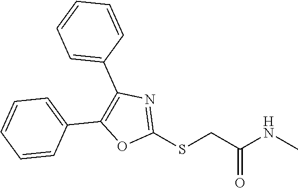 [SMILES: CNC(=O)CSc1nc(-c2ccccc2)c(-c2ccccc2)o1] | 2-(4,5- diphenyloxazol- 2-yl)sulfanyl-N- methyl- acetamide |
| A-16 | 117 | 4.27 | 2.3 | 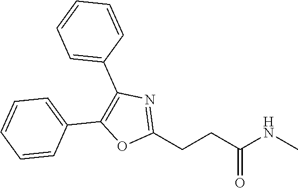 [SMILES: CNC(=O)CCc1nc(-c2ccccc2)c(-c2ccccc2)o1] | 3-(4,5- diphenyloxazol- 2-yl)-N-methyl- propanamide |
| A-17 | 91 | 5.85 | | 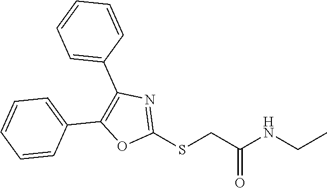 [SMILES: CCNC(=O)CSc1nc(-c2ccccc2)c(-c2ccccc2)o1] | 2-(4,5- diphenyloxazol- 2-yl)sulfanyl-N- ethyl-acetamide |
| A-18 | 80 | | 0.71 | 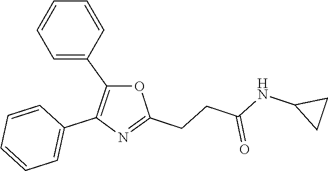 [SMILES: O=C(CCc1nc(-c2ccccc2)c(-c2ccccc2)o1)NC1CC1] | N-cyclopropyl-3- (4,5- diphenyloxazol- 2- yl)propanamide |
| A-19 | 75 | | | 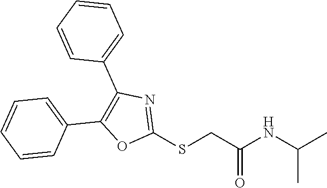 [SMILES: CC(C)NC(=O)CSc1nc(-c2ccccc2)c(-c2ccccc2)o1] | 2-(4,5- diphenyloxazol- 2-yl)sulfanyl-N- isopropyl- acetamide |
| A-20 | 73 | | 0.81 | 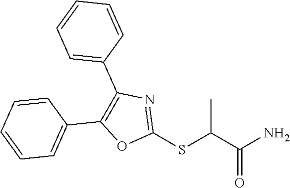 [SMILES: CC(Sc1nc(-c2ccccc2)c(-c2ccccc2)o1)C(N)=O] | 2-(4,5- diphenyloxazol- 2- yl)sulfanyl- propanamide |
| A-21 | 70 | | | 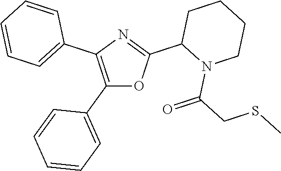 [SMILES: CSCC(=O)N1CCCCC1c1nc(-c2ccccc2)c(-c2ccccc2)o1] | 1-[2-(4,5- diphenyloxazol- 2-yl)-1- piperidyl]-2- methylsulfanyl- ethanone |
| A-22 | 69 | | 0.8 | 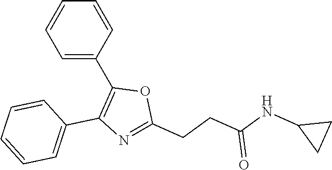 [SMILES: O=C(CCc1nc(-c2ccccc2)c(-c2ccccc2)o1)NC1CC1] | N-cyclopropyl-3- (4,5- diphenyloxazol- 2- yl)propanamide |
| A-23 | 62 | | | 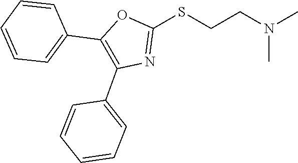 [SMILES: CN(C)CCSc1nc(-c2ccccc2)c(-c2ccccc2)o1] | 2-(4,5- diphenyloxazol- 2-yl)sulfanyl- N,N-dimethyl- ethanamine |
| A-24 | 56 | 11.27 | | 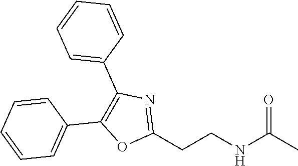 [SMILES: CC(=O)NCCc1nc(-c2ccccc2)c(-c2ccccc2)o1] | N-[2-(4,5- diphenyloxazol- 2- yl)ethyl] acetamide |
| A-25 | 55 | | | 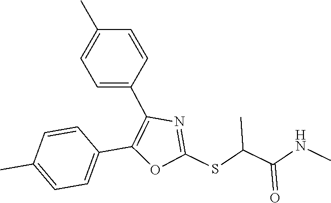 | 2-[4,5-bis (p- tolyl)oxazol-2- yl]sulfanyl-N- methyl- propanamide |
| A-26 | 51 | | | 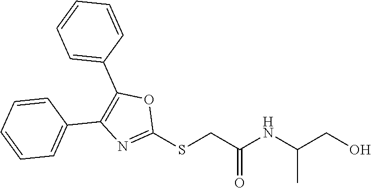 [SMILES: CC(CO)NC(=O)CSc1nc(-c2ccccc2)c(-c2ccccc2)o1] | 2-(4,5- diphenyloxazol- 2-yl)sulfanyl-N- (2-hydroxy-1- methyl- ethyl)acetamide |
| A-27 | 42 | 18.04 | | 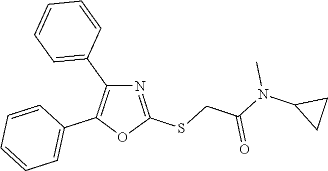 [SMILES: CN(C(=O)CSc1nc(-c2ccccc2)c(-c2ccccc2)o1)C1CC1] | N-cyclopropyl-2- (4,5- diphenyloxazol- 2-yl)sulfanyl-N- methyl- acetamide |
| A-28 | 40 | | | 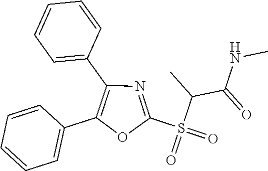 [SMILES: CNC(=O)C(C)S(=O)(=O)c1nc(-c2ccccc2)c(-c2ccccc2)o1] | 2-(4,5- diphenyloxazol- 2-yl)sulfonyl-N- methyl- propanamide |
| A-29 | 36 | | | 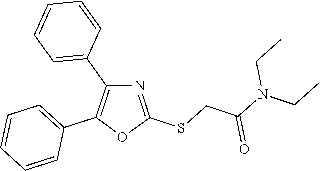 [SMILES: CCN(CC)C(=O)CSc1nc(-c2ccccc2)c(-c2ccccc2)o1] | 2-(4,5- diphenyloxazol- 2-yl)sulfanyl- N,N-diethyl- acetamide |
| A-30 | 26 | | | 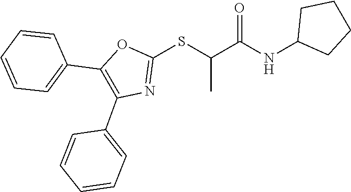 [SMILES: CC(Sc1nc(-c2ccccc2)c(-c2ccccc2)o1)C(=O)NC1CCCC1] | N-cyclopentyl-2- (4,5- diphenyloxazol- 2-yl)sulfanyl- propanamide |
| A-31 | 24 | | | 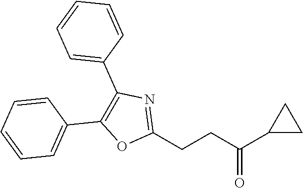 [SMILES: O=C(CCc1nc(-c2ccccc2)c(-c2ccccc2)o1)C1CC1] | 1-cyclopropyl-3- (4,5- diphenyloxazol- 2-yl)propan-1- one |
| A-32 | 23 | | | 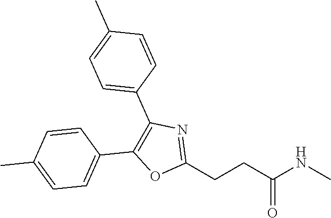 | 3-[4,5-bis (p- tolyl)oxazol-2- yl]-N-methyl- propanamide |
| A-33 | 21 | | | 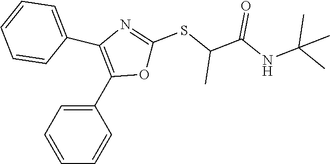 [SMILES: CC(Sc1nc(-c2ccccc2)c(-c2ccccc2)o1)C(=O)NC(C)(C)C] | N-tert-butyl-2- (4,5- diphenyloxazol- 2-yl) sulfanyl- propanamide |
| A-34 | 16 | | | 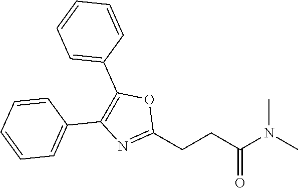 [SMILES: CN(C)C(=O)CCc1nc(-c2ccccc2)c(-c2ccccc2)o1] | 3-(4,5- diphenyloxazol- 2-yl)-N,N- dimethyl- propanamide |
| A-35 | 13 | | | 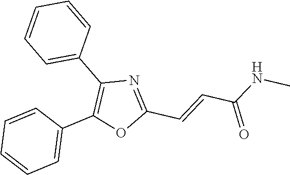 [SMILES: CNC(=O)/C=C/c1nc(-c2ccccc2)c(-c2ccccc2)o1] | (E)-3-(4,5- diphenyloxazol- 2-yl)-N-methyl- prop-2-enamide |
| A-36 | 12 | | | 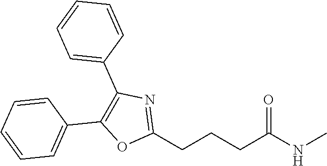 [SMILES: CNC(=O)CCCc1nc(-c2ccccc2)c(-c2ccccc2)o1] | 4-(4,5- diphenyloxazol- 2-yl)-N-methyl- butanamide |
| A-37 | 7 | | | 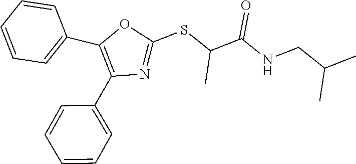 [SMILES: CC(C)CNC(=O)C(C)Sc1nc(-c2ccccc2)c(-c2ccccc2)o1] | 2-(4,5- diphenyloxazol- 2-yl)sulfanyl-N- isobutyl- propanamide |
| A-38 | 6 | | | 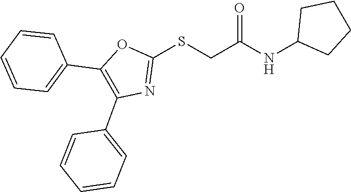 [SMILES: O=C(CSc1nc(-c2ccccc2)c(-c2ccccc2)o1)NC1CCCC1] | N-cyclopentyl-2- (4,5- diphenyloxazol- 2-yl)sulfanyl- acetamide |
| A-39 | 5 | | 0.7 | 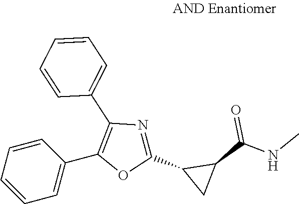 [SMILES: CNC(=O)[C@H]1C[C@@H]1c1nc(-c2ccccc2)c(-c2ccccc2)o1] | rac-(1S,2S)-2- (4,5- diphenyloxazol- 2-yl)-N-methyl- cyclopropane- carboxamides |
| A-40 | 5 | | | 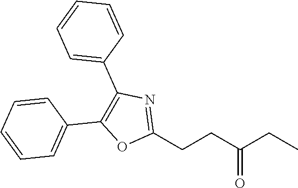 [SMILES: CCC(=O)CCc1nc(-c2ccccc2)c(-c2ccccc2)o1] | 1-(4,5- diphenyloxazol- 2-yl)pentan-3- one |
| A-42 | 4 | | | 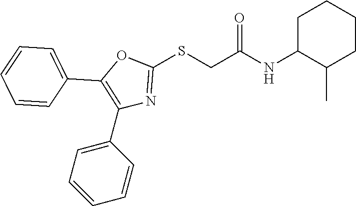 [SMILES: CC1CCCCC1NC(=O)CSc1nc(-c2ccccc2)c(-c2ccccc2)o1] | 2-(4,5- diphenyloxazol- 2-yl)sulfanyl-N- (2- methylcyclohexyl) acetamide |
| A-43 | 3 | | | 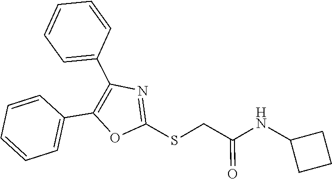 [SMILES: O=C(CSc1nc(-c2ccccc2)c(-c2ccccc2)o1)NC1CCC1] | N-cyclobutyl-2- (4,5- diphenyloxazol- 2-yl)sulfanyl- acetamide |
| A-44 | 3 | | | 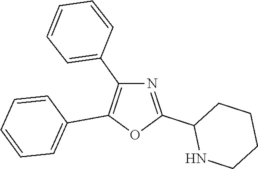 [SMILES: c1ccc(-c2nc(C3CCCCN3)oc2-c2ccccc2)cc1] | 4,5-diphenyl-2- (2- piperidyl)oxazole |
| A-45 | 2 | | | 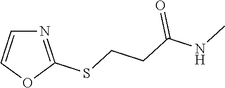 | N-methyl-3- oxazol-2- ylsulfanyl- propanamide |
| A-46 | 2 | | | 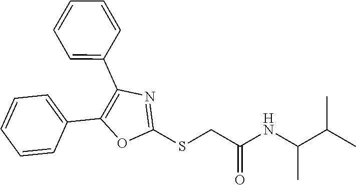 [SMILES: CC(C)C(C)NC(=O)CSc1nc(-c2ccccc2)c(-c2ccccc2)o1] | N-(1,2- dimethylpropyl)- 2-(4,5- diphenyloxazol- 2-yl)sulfanyl- acetamide |
| A-47 | 2 | | | 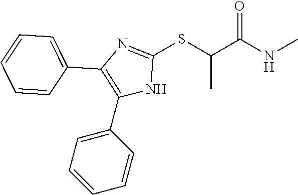 [SMILES: CNC(=O)C(C)Sc1nc(-c2ccccc2)c(-c2ccccc2)[nH]1] | 2-[(4,5-diphenyl- 1H-imidazol-2- yl)sulfanyl]-N- methyl- propanamide |
| A-48 | 2 | | | 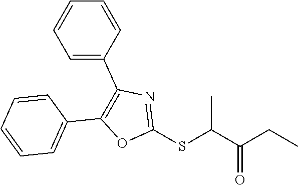 [SMILES: CCC(=O)C(C)Sc1nc(-c2ccccc2)c(-c2ccccc2)o1] | 2-(4,5- diphenyloxazol- 2- yl) sulfanylpentan- 3-one |
| A-49 | 2 | | | 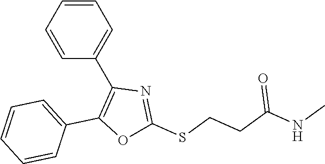 [SMILES: CNC(=O)CCSc1nc(-c2ccccc2)c(-c2ccccc2)o1] | 3-(4,5- diphenyloxazol- 2-yl)sulfanyl-N- methyl- propanamide |
| A-50 | 1 | | | 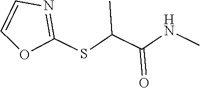 | N-methyl-2- oxazol-2- ylsulfanyl- propanamide |
| A-51 | 1 | | | 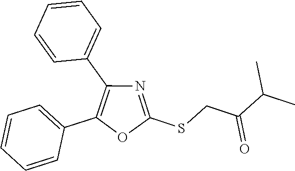 [SMILES: CC(C)C(=O)CSc1nc(-c2ccccc2)c(-c2ccccc2)o1] | 1-(4,5- diphenyloxazol- 2-yl)sulfanyl-3- methyl-butan-2- one |
| A-52 | 1 | | | 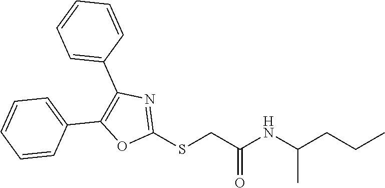 [SMILES: CCCC(C)NC(=O)CSc1nc(-c2ccccc2)c(-c2ccccc2)o1] | 2-(4,5- diphenyloxazol- 2-yl)sulfanyl-N- (1- methylbutyl) acetamide |
| A-53 | 1 | | | 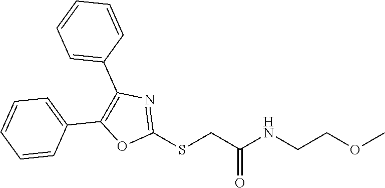 [SMILES: COCCNC(=O)CSc1nc(-c2ccccc2)c(-c2ccccc2)o1] | 2-(4,5- diphenyloxazol- 2-yl)sulfanyl-N- (2- methoxyethyl) acetamide |
| A-54 | 1 | | | 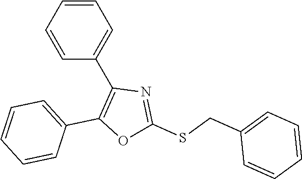 [SMILES: c1ccc(CSc2nc(-c3ccccc3)c(-c3ccccc3)o2)cc1] | 2- benzylsulfanyl- 4,5-diphenyl- oxazole |
| A-55 | 1 | | 0.59 | 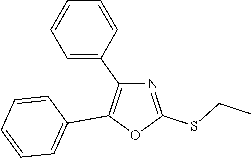 [SMILES: CCSc1nc(-c2ccccc2)c(-c2ccccc2)o1] | 2-ethylsulfanyl- 4,5-diphenyl- oxazole |
| A-56 | 1 | | | 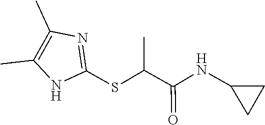 [SMILES: Cc1nc(SC(C)C(=O)NC2CC2)[nH]c1C] | N-cyclopropyl-2- [(4,5-dimethyl- 1H-imidazol-2- yl)sulfanyl] propanamide |
| A-57 | 1 | | | 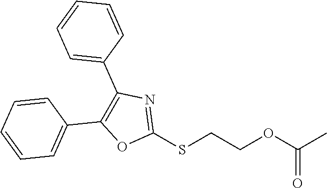 [SMILES: CC(=O)OCCSc1nc(-c2ccccc2)c(-c2ccccc2)o1] | 2-(4,5- diphenyloxazol- 2- yl)sulfanylethyl acetate |
| A-58 | 1 | | | 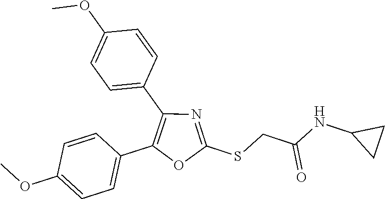 [SMILES: COc1ccc(-c2nc(SCC(=O)NC3CC3)oc2-c2ccc(OC)cc2)cc1] | 2-[4,5-bis(4- methoxyphenyl) oxazol-2- yl]sulfanyl-N- cyclopropyl- acetamide |
| A-59 | 1 | | | 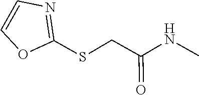 | N-methyl-2- oxazol-2- ylsulfanyl- acetamide |
| A-60 | 0 | | | 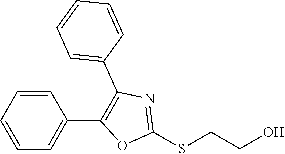 [SMILES: OCCSc1nc(-c2ccccc2)c(-c2ccccc2)o1] | 2-(4,5- diphenyloxazol- 2- yl) sulfanylethanol |
| A-61 | 0 | | | 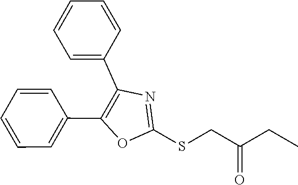 [SMILES: CCC(=O)CSc1nc(-c2ccccc2)c(-c2ccccc2)o1] | 1-(4,5- diphenyloxazol- 2- yl)sulfanylbutan- 2-one |
| A-62 | 0 | | | 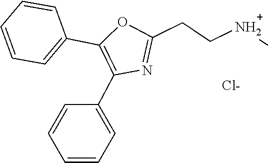 [SMILES: C[NH2+]CCc1nc(-c2ccccc2)c(-c2ccccc2)o1 ; [Cl-]] | 2-(4,5- diphenyloxazol- 2-yl)ethyl- methyl- ammonium; chloride |
| A-63 | −1 | | | 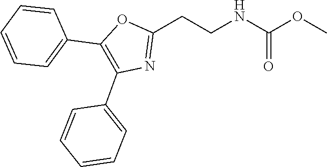 [SMILES: COC(=O)NCCc1nc(-c2ccccc2)c(-c2ccccc2)o1] | methyl N-[2- (4,5- diphenyloxazol- 2- yl)ethyl]carbamate |
| A-64 | −1 | | | 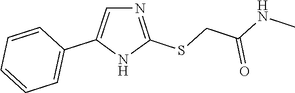 [SMILES: CNC(=O)CSc1ncc(-c2ccccc2)[nH]1] | N-methyl-2-[(5- phenyl-1H- imidazol-2- yl) sulfanyll- acetamide |
| A-65 | −1 | | | 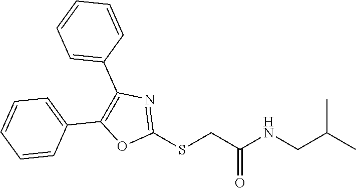 [SMILES: CC(C)CNC(=O)CSc1nc(-c2ccccc2)c(-c2ccccc2)o1] | 2-(4,5- diphenyloxazol- 2-yl)sulfanyl-N- isobutyl- acetamide |
| A-66 | −1 | | | | N-cyclopropyl-2- oxazol-2- ylsulfanyl- acetamide |
| A-67 | −2 | | | 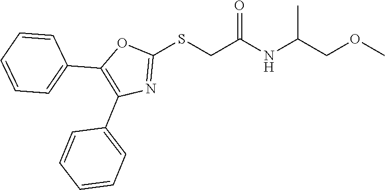 [SMILES: COCC(C)NC(=O)CSc1nc(-c2ccccc2)c(-c2ccccc2)o1] | 2-(4,5- diphenyloxazol- 2-yl)sulfanyl-N- (2-methoxy-1- methyl- ethyl)acetamide |
| A-68 | −2 | | | 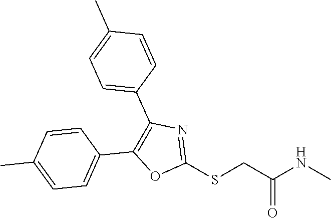 | 2-[4,5-bis(p- tolyl)oxazol-2- yl]sulfanyl-N- methyl- acetamide |
| A-69 | −2 | | | 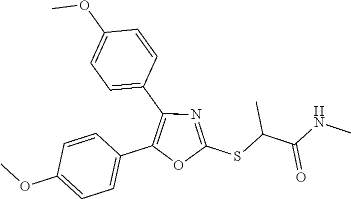 | 2-[4,5-bis(4- methoxyphenyl) oxazol-2- yl]sulfanyl-N- methyl- propanamide |
| A-70 | −2 | | | 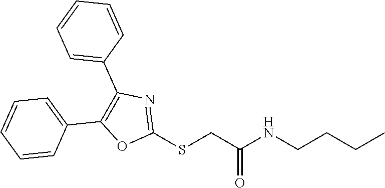 [SMILES: CCCCNC(=O)CSc1nc(-c2ccccc2)c(-c2ccccc2)o1] | N-butyl-2-(4,5- diphenyloxazol- 2-yl)sulfanyl- acetamide |
| A-71 | −3 | | | 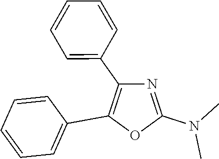 [SMILES: CN(C)c1nc(-c2ccccc2)c(-c2ccccc2)o1] | N,N-dimethyl- 4,5-diphenyl- oxazol-2-amine |
| A-72 | −5 | | | 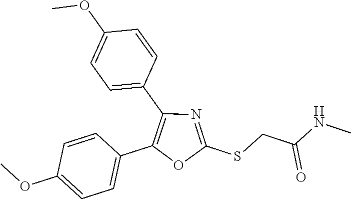 | 2-[4,5-bis(4- methoxyphenyl) oxazol-2- yl]sulfanyl-N- methyl- acetamide |
| A-73 | −5 | | | 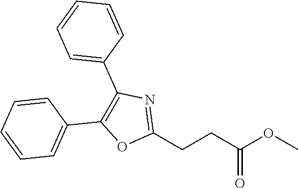 [SMILES: COC(=O)CCc1nc(-c2ccccc2)c(-c2ccccc2)o1] | methyl 3-(4,5- diphenyloxazol- 2-yl)propanoate |

Of the above compounds, the use of compounds B-01, B-02, B-03, B-04, B-05, B-06, B-07, B-11, B14, B-15, B-17, B-18, B-19 and B-21 and the use of compounds A-01, A-02, A-03, A-04, A-05, A-06, A-07, A-08, A-09, A-10, A-11, A-12, A-15, A-16 and A-17 are preferred because of their pronounced TRPM8 activation, their EC50 value and their cooling intensity. Most preferred, however, is the use of one of the compounds B-01, B-02, B-03, B-04, B-05, B-06, B-07 and B-11 and the use of one of the compounds A-01, A-02, A-03, A-04, A-05, A-06, A-07, A-08, A-09, A-10, A-11 and A-12 because of their outstanding TRPM8 activation, their EC50 value and their cooling intensity.

In a further aspect, the present invention therefore also comprises food, nutritional supplements, cosmetic or pharmaceutical preparations, animal feed, textiles, packaging or tobacco products comprising a physiological coolant or a physiological coolant mixture according to the invention or a flavoring preparation according to the invention.

The content of the one or more coolants depends on the type and use of the aforementioned products and is preferably about 0.1 ppm to 10% by weight, preferably 1% to 10% by weight, based on the total weight of the end product. In oral care applications, for example in toothpastes or mouthwashes, the content is 0.1 ppm to 500 ppm of one or more coolants.

A broad concentration range typically used to provide the desired level of sensitivity modulation may be about 0.001 ppm to 1000 ppm, or about 0.01 ppm to about 500 ppm, or about 0.05 ppm to about 300 ppm, or about 0.1 ppm to about 200 ppm, or about 0.5 ppm to about 150 ppm, or about 1 ppm to about 100 ppm.

Preferably, the foodstuffs are bakery products, for example bread, dry cookies, cakes, other baked goods, confectionery (for example chocolates, chocolate bar products, other bar products, fruit gums, hard and soft caramels, chewing gum), alcoholic or non-alcoholic drinks (e.g. coffee, tea, iced tea, wine, wine-based drinks, beer, beer-based drinks, liqueurs, schnapps, brandies, (carbonated) fruit-based soft drinks, (carbonated) isotonic drinks, (carbonated) soft drinks, nectars, spritzers, fruit and vegetable juices, fruit or vegetable juice preparations, instant drinks (e.g. instant cocoa drinks, instant tea drinks, instant coffee drinks, instant fruit drinks), meat products (e.g. ham, fresh sausage or raw sausage preparations, seasoned or marinated fresh or cured meat products), eggs or egg products (dried eggs, egg whites, egg yolks), cereal products (e.g. breakfast cereals, muesli bars, pre-cooked ready-to-eat rice products), dairy products (e.g. milk drinks, buttermilk drinks, milk ice cream, yoghurt, kefir, cream cheese, soft cheese, hard cheese, dried milk powder, whey, whey drinks, butter, buttermilk, partially or fully hydrolyzed milk protein-containing products), products made from soy protein or other soybean fractions (e.g. soy milk and products made from it, fruit drinks with soy protein, preparations containing soy lecithin, fermented products such as tofu or tempeh or products made from them), products made from other vegetable protein sources, for example oat protein drinks, fruit preparations (for example jams, fruit ice cream, fruit sauces, fruit fillings), products made from soy protein or other soybean fractions (for example soy milk and products made from it), fruit preparations (e.g. jams, fruit ice cream, fruit sauces, fruit fillings), vegetable preparations (e.g. ketchup, sauces, dried vegetables, frozen vegetables, pre-cooked vegetables, boiled vegetables), snacks (e.g. baked or deep-fried potato potato chips or potato dough products, corn or peanut-based extrudates), fat- and oil-based products or emulsions thereof (e.g. mayonnaise, remoulade, dressings), other ready meals and soups (e.g. dry soups, instant soups, pre-cooked soups), spices, seasoning mixtures and, in particular, seasonings, which are used, for example, in snack foods: Seasonings), which are used in the snack sector, for example.

In addition to conventional food ingredients, the above-mentioned foods contain at least one effective, i.e. cooling, amount of at least one coolant according to the invention or a coolant mixture according to the invention or a flavoring preparation according to the invention.

The content of coolant or coolant mixture or flavoring preparation in these preparations is preferably about 0.1 wt. % to about 10 wt. % and in particular about 1 wt. % to 2 wt. %, based on the total weight of the finished preparation.

Suitable excipients can be used to manufacture the products according to the invention, such as foodstuffs, food supplements, cosmetic or pharmaceutical preparations, animal feed, textiles, packaging or tobacco products. Suitable excipients include, but are not limited to, emulsifiers, thickeners, food acids, acidity regulators, vitamins, antioxidants, flavor enhancers, active ingredients for masking unpleasant taste impressions, food colorants and the like.

In particular, other conventional additives or auxiliary substances can be added to the above-mentioned products according to the invention, such as flavorings or active ingredients for masking unpleasant taste impressions.

Flavoring agents: Preferred flavoring agents are those which cause a sweet odor impression, wherein the further flavoring agent(s) which cause a sweet odor impression are preferably selected from the group consisting of: Vanillin, ethyl vanillin, ethyl vanillin isobutyrate (=3 ethoxy-4-isobutyryloxybenzaldehyde), furaneol (2,5-dimethyl-4-hydroxy-3(2H)-furanone) and derivatives (e.g. homofuraneol, 2-ethyl-4-hydroxy-5-methyl-3(2H)-furanone), homofuronol (2-ethyl-5-methyl-4-hydroxy-3(2H)-furanone and 5-ethyl-2-methyl-4-hydroxy-3(2H)-furanone), maltol and derivatives (e.g. ethylmaltol), coumarin and derivatives, gamma-lactones (e.g. gamma-undecalactone, gamma-nonalactone), delta-lactones (e.g. 4-methyldeltalactone, massoilactone, deltadecalactone, tuberolactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, fruit esters and fruit lactones (e. g. B. Acetic acid n-butyl ester, acetic acid isoamyl ester, propionic acid ethyl ester, butyric acid ethyl ester, butyric acid n-butyl ester, butyric acid i-soamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl 3-methyl-3-phenyl glycidate, ethyl 2-trans-4-cis-decadienoate), 4-(p-hydroxyphenyl)-2-butanone, 1,1-dimethoxy-2,2,5-trimethyl-4-hexane, 2,6-dimethyl-5-hepten-1-al, 4-hydroxycinnamic acid, 4-methoxy-3-hydroxycinnamic acid, 3-methoxy-4-hydroxycinnamic acid, 2-hydroxycinnamic acid, 2,4-dihydroxybenzoic acid, 3-hydroxybenzoic acid, 3,4-dihydroxybenzoic acid, vanillic acid, homovanillic acid, vanillomandelic acid and phenylacetaldehyde.

Active ingredients for masking unpleasant taste sensations: Furthermore, the oral preparations may also comprise further substances which also serve to mask bitter and/or astringent taste impressions. These further flavor corrigents are selected, for example, from the following list: nucleotides (e.g. adenosine 5′-monophosphate, cytidine 5′-monophosphate) or physiologically acceptable salts thereof, lactisols, sodium salts (e.g. sodium chloride, sodium laurate), sodium salts (e.g. sodium chloride, sodium lactate, sodium citrate, sodium acetate, sodium gluconoate), hydroxyflavanones, preferably eriodictyol, sterubin (eriodictyol-7-methylether), homoeriodictyol, and their sodium, potassium, calcium, magnesium or zinc salts (e.g. sodium chloride, sodium lactate, sodium citrate, sodium acetate, sodium gluconoate), magnesium or zinc salts thereof (in particular those as described in EP 1258200 A2), hydroxybenzoic acid amides, preferably 2,4-dihydroxybenzoic acid vanillylamide, 2,4-dihydroxybenzoic acid N-(4-hydroxy-3-methoxybenzyl)amide, 2,4,6-trihydroxybenzoic acid-N-(4-hydroxy-3-methoxybenzyl)amide, 2-hydroxybenzoic acid-N-4-(hydroxy-3-methoxybenzyl)amide, 4-hydroxybenzoic acid-N-(4-hydroxy-3-methoxybenzyl)amide, 2,4-dihydroxybenzoic acid-N-(4-hydroxy-3-methoxybenzyl)amide mono-sodium salt, 2,4-dihydroxybenzoic acid-N-2-(4-hydroxy-3-methoxy-phenyl)ethylamide, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N-[2-(4-hydroxy-3-methoxyphenyl)ethyl]amide; 4-hydroxybenzoic acid vanillylamides (in particular those as described in WO 2006/024587); hydroxydeoxybenzoines, preferably 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)-ethanone and 1-(2-hydroxy-4-methoxyphenyl)-2-(4-hydroxy-3-methoxyphenyl)ethanone) (in particular those as described in WO 2006/106023); hydroxyphenylalkanediones, such as gingerdione-[2], gingerdione-[3], gingerdione-[4], dehydrogingerdione-[2], dehydrogingerdione-[3], dehydrogingerdione-[4]) (in particular those as described in WO 2007/003527); diacetyltrimers (in particular those as described in WO 2006/058893); gamma-aminobutyric acids (in particular those as described in WO 2005/096841); divanillins (in particular those as described in WO 2004/078302) and 4-hydroxydihydrochalcones (preferably as described in US 2008/0227867 A1), in particular phloretin and Davidigenin, amino acids or mixtures of whey proteins with lecithins, hesperetin as disclosed in WO 2007/014879, 4-hydroxydihydrochalcones as disclosed in WO 2007/107596, or propenylphenylglycosides (chavicolglycosides) as described in EP 1955601 A1, or extracts from Rubus suavissimus, extracts from Hydrangea macrophylla as described in EP 2298084 A1, pellitorin and derived aroma compositions as described in EP 2008530 A1, umami compounds as described in WO 2008/046895 A1 and EP 1989944 A1, umami compounds as described in EP 2064959 A1 and EP 2135516 A1, vanillyllignans, enterodiol, and N-decadienoylamino acids and mixtures thereof.

A further object of the invention relates to cosmetic or pharmaceutical preparations which contain either one or more of the coolant(s) according to the invention or a coolant mixture according to the invention or a flavoring preparation according to the invention.

The agents according to the invention can be, in particular, skin cosmetic, hair cosmetic, dermatological, hygienic or pharmaceutical agents. In particular, the active ingredients according to the invention, which have a cooling effect in particular, are used for skin and/or hair cosmetics or as oral care products.

The hair- or skin-care compositions or preparations according to the invention are preferably in the form of an emulsion, a dispersion, a suspension, in the form of an aqueous surfactant preparation, a milk, a lotion, a cream, a balm, an ointment, a gel, a granulate, a powder, a stick preparation, such as a lipstick, a foam, an aerosol or a spray. Such formulations are well suited for topical preparations. Suitable emulsions are oil-in-water emulsions and water-in-oil emulsions or microemulsions. As a rule, the hair or skin cosmetic preparation is used for application to the skin (topical) or hair. The term “topical preparations” refers to preparations that are suitable for applying the active ingredients to the skin in a finely dispersed form, e.g. in a form that can be absorbed through the skin. Aqueous and aqueous-alcoholic solutions, sprays, foams, foam aerosols, ointments, aqueous gels, emulsions of the O/W or W/O type, microemulsions or cosmetic stick preparations are suitable for this purpose, for example. According to one embodiment of the cosmetic composition according to the invention, this contains a carrier. The preferred carrier is water, a gas, a water-based liquid, an oil, a gel, an emulsion or microemulsion, a dispersion or a mixture thereof. The carriers mentioned show good skin compatibility. Aqueous gels, emulsions or microemulsions are particularly advantageous for topical preparations.

The teaching according to the invention also includes the use of the active ingredients described herein for medical purposes, in particular in pharmaceutical compositions for the treatment of an individual, preferably a mammal, in particular a human, farm animal or domestic animal. For this purpose, the active ingredients are administered in the form of pharmaceutical compositions comprising a pharmaceutically acceptable excipient with at least one active ingredient according to the invention and optionally further active ingredients. These compositions can be administered, for example, by the oral, rectal, transdermal, subcutaneous, intravenous, intramuscular or intranasal route.

Examples of suitable pharmaceutical formulations or Compositions include solid dosage forms such as powders, powders, granules, tablets, pastilles, sachets, cachets, coated tablets, capsules such as hard and soft gelatine capsules, suppositories or vaginal dosage forms, semi-solid dosage forms such as ointments, creams, hydrogels, pastes or patches, and liquid dosage forms such as solutions, emulsions, in particular oil-in-water emulsions, suspensions, creams, hydrogels, pastes or patches, creams, hydrogels, pastes or patches, as well as liquid dosage forms such as solutions, emulsions, in particular oil-in-water emulsions, suspensions, for example lotions, injection and infusion preparations, eye and ear drops. Implanted delivery devices can also be used to administer inhibitors according to the invention. Furthermore, liposomes, microspheres or polymer matrices can also be used. Possible pharmaceutical agents include cold syrups, wound ointments or wound sprays. It is also possible to incorporate the substances into plasters or tablets, especially if these contain active ingredients that themselves have an unpleasant taste.

A further object of the present invention therefore comprises the coolants or coolant mixtures according to the invention as medicaments, in particular as medicaments for use in relieving pain and inflammatory conditions of the skin and mucous membranes. Due to their cooling properties, the coolants according to the invention are particularly suitable for preventing, combating or alleviating symptoms of coughs, colds, inflammation, sore throats or hoarseness.

The substances and preparations described herein are also suitable for treating inflammatory conditions of the skin, mucous membranes and joints due to their effective cooling effect.

Due to their properties to modulate the receptor TRPM8, whose gene expression, i.e. that of the TRPM8 gene, is upregulated in cancer diseases, for example in prostate carcinomas, the pharmaceutical preparations according to the invention are preferably also used in oncology, preferably in the treatment of prostate or bladder carcinomas, or for the treatment of bladder weakness. The corresponding proteins in the cell are encoded by corresponding genes in the cell nucleus. Reading the genes in the nucleus (transcription) leads to the generation of messenger RNA (mRNA), which is then “translated” into a protein in the cell on ribosomes (translation). The totality of both processes is often referred to as gene expression.

However, astringent, bitter and/or metallic tastes are not only found in flavors and sweeteners as described above, but also in connection with many active pharmaceutical ingredients, which makes them difficult to take, especially in children. Typical examples of such active pharmaceutical ingredients are the following: Aspirin, minoxidil, erythromycin, fenistil, betamethasone, ibuprofen, ketoprofen, dicyclofenac, metronidazole, acyclovir, imiquimod, terbafine, cyclopiroxolamine, paracetamol, and other pharmaceutical agents of the non-steroidal anti-inflammatory drug (NSAID) type and mixtures thereof.

The present invention therefore also comprises medicaments which contain one or more coolants according to the invention or a coolant mixture according to the invention or a flavoring preparation according to the invention in combination with at least one further active pharmaceutical ingredient selected from the group consisting of aspirin, minoxidil, erythromycin, fenistil, betamethasone, ibuprofen, ketoprofen, dicyclofenac, metronidazole, acyclovir, imiquimod, terbafine, cyclopiroxolamine, paracetamol and mixtures thereof.

It has also been shown in subject studies that the coolants according to the invention or the coolant mixtures according to the invention enhance the pain-reducing properties of non-steroidal anti-inflammatory substances (NSAIDs), in particular ibuprofen and ketoprofen, beyond the cooling effect, which was also not expected by the skilled person. Therefore, the present invention also relates in particular to the combination with pharmaceutical agents of the non-steroidal anti-inflammatory drug (NSAID) type.

Such pharmaceutical combinations are therefore particularly advantageous for use in the treatment of inflammatory conditions of the skin and mucous membranes as well as the joints.

The medicaments may contain the coolants according to the invention or the coolant mixtures according to the invention and the active pharmaceutical ingredients in a weight ratio of about 1:99 to about 10:90 and in particular 2:98 to about 5:95.

The physiological cooling effect is also used, for example, in the formulation of ointments for wounds and burns as well as preparations for insect bites.

In the preparation of the cosmetic or pharmaceutical compositions according to the invention, the coolant(s) or the coolant mixture according to the invention are usually mixed or diluted with an excipient. Excipients can be solid, semi-solid or liquid materials that serve as a vehicle, carrier or medium for the active ingredient. The active ingredient content (of one or more simultaneously contained coolants according to the invention) can vary over a wide range and is approximately, in each case based on the total weight of the preparation, from about 0.05 ppm to 10% by weight, preferably 0.1 ppm to 10% by weight.

Suitable excipients include, for example, lactose, dextrose, sucrose, sorbitol, mannitol, starches, acacia gum, calcium phosphate, alginates, tragacanth, gelatin, calcium silicate, microcrystalline cellulose, polyvinylpyrrolidone, cellulose, water, syrup and methylcellulose. Furthermore, the formulations may contain pharmaceutically acceptable carriers or conventional excipients, such as lubricants, for example tallow, magnesium stearate and mineral oil, wetting agents, emulsifying and suspending agents, preservatives, such as methyl and propyl hydroxybenzoates; antioxidants; anti-irritants; chelating agents; coating agents; emulsion stabilizers; film formers; gel formers; odour masking agents; taste corrigents; resins; hydrocolloids; solvents; solubilizers; neutralizing agents; permeation accelerators; pigments; quaternary ammonium compounds; refatting and superfatting agents; ointment, cream or oil bases; silicone derivatives; spreading aids; stabilizers; sterilants; suppository bases; tablet excipients, such as binders, fillers, lubricants, disintegrants or coatings; propellants; drying agents; opacifiers; thickeners; waxes; plasticizers; white oils. A relevant embodiment is based on expert knowledge and is adequately described in the relevant technical literature.

The preparations according to the invention may also contain cosmetically and/or dermatologically and/or pharmacologically active agents in addition to conventional additives or auxiliaries. Non-limiting examples of suitable further active ingredients are: cosmetically and/or dermatologically active ingredients; antimicrobial active ingredients; surfactants (anionic surfactants, nonionic surfactants, cationic surfactants, amphoteric or zwitterionic surfactants), oil bodies, emulsifiers, emulsifying agents, antimicrobial agents and other active ingredients. zwitterionic surfactants), oil bodies, emulsifiers, fats and waxes, pearlescent waxes, consistency agents and thickeners, superfatting agents and stabilizers, polymers, silicone compounds, UV light protection filters, pigments, especially light protection pigments, humectants, Biogenic active ingredients and antioxidants, deodorants and germ inhibitors, enzyme inhibitors, odour absorbers, antiperspirants, film-forming agents, anti-dandruff agents, swelling agents, insect repellents, hydrotropes, preservatives, perfume oils and aromas, dyes, etc.

Preferred preparations according to the invention are selected from the group of products for the treatment, protection, care and cleansing of the skin and/or hair or as a make-up product, either as leave-on or rinse-off products.

The formulations include, for example, dispersions, suspensions, creams, lotions or milks, depending on the manufacturing method and ingredients, gels (including hydrogels, e.g. hydrodispersion gels, oleogels), sprays (e.g. pump sprays or sprays with propellant), foams or impregnating solutions for cosmetic wipes, soaps, washing liquids, shower and bath preparations, bath products (capsules, oil, tablets). foams or impregnating solutions for cosmetic wipes, soaps, washing liquids, shower and bath preparations, bath products (capsules, oil, tablets, salts, bath salts, soaps, etc.), effervescent preparations, skin care products, such as emulsions, ointments, pastes, gels (as described above), oils, balms, serums, powders (e.g. face powder, body powder), masks, sticks, roll-on sticks, aerosols (foaming, non-foaming or after-foaming), deodorants and/or antiperspirants, mouthwashes and mouth rinses, insect repellents, sunscreens, after-sun products, shaving products, aftershave balms, pre- and aftershave lotions, depilatories, hair care products such as Shampoos (including 2-in-1 shampoos, anti-dandruff shampoos, baby shampoos, shampoos for dry scalps, concentrated shampoos), conditioners, hair tonics, hair lotions, hair rinses, styling creams, pomades, perming and setting lotions, hair sprays, e.g. styling aids (e.g. gel or wax), hair straightening agents (detangling agents, relaxers), hair dyes such as temporary hair dyes, semi-permanent hair dyes, permanent hair dyes, hair conditioners, hair foams, eye care products, make-up, make-up removers or baby products.

Particularly preferably, the formulations according to the invention are in the form of an emulsion, in particular in the form of a W/O, O/W, W/O/W, O/W/O emulsion, PIT emulsion, e.g. a Pickering emulsion, an emulsion with a low oil content, a micro- or nanoemulsion, a gel (including hydrogel, hydrodispersion gel, oleogel) or a solution.

The total proportion of excipients and additives can be 1 wt. % to 50 wt. %, preferably 5 wt. % to 40 wt. %, based on the final preparation. The preparation of the agents can be carried out by conventional cold or hot processes; preferably the phase inversion temperature method is used.

The present invention also includes oral care compositions comprising one or more coolants according to the invention or a coolant mixture according to the invention or a flavoring preparation according to the invention.

Oral hygiene products according to the invention can be formulated in a manner known per se, e.g. as toothpaste, toothpaste, toothpaste gel, tooth powder, toothbrushing liquid, toothbrushing foam, aqueous or aqueous-alcoholic oral care products (mouthwash), mouthwash as a 2-in-1 product, lozenge, mouth spray, dental floss and dental care chewing gum.

Toothpastes or toothpastes are generally understood to be gel-like or pasty preparations of water, thickening agents, humectants, abrasive or cleaning agents, surfactants, sweeteners, flavorings, deodorizing active ingredients and active ingredients against oral and dental diseases. All conventional cleaning agents, such as chalk, dicalcium phosphate, insoluble sodium metaphosphate, aluminum silicates, calcium pyrophosphate, finely divided synthetic resins, silicas, aluminum oxide and aluminum oxide trihydrate can be used in the toothpastes according to the invention.

Preferably suitable cleaning agents for the toothpastes according to the invention are above all finely divided xerogel silicas, hydrogel silicas, precipitated silicas, aluminum oxide trihydrate and finely divided alpha-aluminum oxide or mixtures of these cleaning agents in amounts of 15 to 40% by weight of the toothpaste. Low-molecular polyethylene glycols, glycerol, sorbitol or mixtures of these products in quantities of up to 50% by weight can be used as humectants. Among the known thickening agents are the thickening, finely divided gel silicas and hydrocolloids, e.g. carboxymethylcellulose, hydroxyethylcellulose, hydroxypropylguar, hydroxyethyl starch, polyvinylpyrrolidone, high molecular weight polyethylene glycol, plant gums such as tragacanth, agar-agar, carragheen moss, gum arabic, xantham gum and carboxyvinyl polymers (e.g. Carbopol® types) are suitable. In addition to the mixtures of menthofuran and menthol compounds, the oral and dental care compositions may in particular contain surface-active substances, preferably anionic and non-ionic high-foaming surfactants, such as the substances already mentioned above, but in particular alkyl ether sulphate salts, alkyl polyglucosides and mixtures thereof.

Other common toothpaste additives are:
Preservatives and antimicrobial substances such as p-hydroxybenzoic acid methyl, ethyl or propyl ester, sodium sorbate, sodium benzoate, bromochlorophene, phenyl salicylic acid ester, thymol and the like;
Anti-tartaric agents, e.g. organophosphates such as 1-hydroxyethane-1,1-diphosphonic acid, 1-phosphonopropane-1,2,3-tricarboxylic acid and others, which are known, for example, from U.S. Pat. No. 3,488,419, DE 2224430 A1 and DE 2343196 A1;
other caries-inhibiting substances such as sodium fluoride, sodium monofluorophosphate, stannous fluoride;
Sweeteners such as saccharin sodium, sodium cyclamate, sucrose, lactose, maltose, fructose or Apartam® (L-aspartyl-L-phenylalanine methyl ester), stivia extracts or their sweetening components, in particular ribeaudiosides;
Additional flavors such as eucalyptus oil, anise oil, fennel oil, caraway oil, methyl acetate, cinnamaldehyde, anethole, vanillin, thymol and mixtures of these and other natural and synthetic flavors;
Pigments such as titanium dioxide;
Colorants;
Buffer substances such as primary, secondary or tertiary alkali phosphates or citric acid/sodium citrate;
wound-healing and anti-inflammatory substances such as allantoin, urea, azulene, chamomile active ingredients and acetylsalicylic acid derivatives.

Hydrotropes such as ethanol, isopropyl alcohol or polyols can also be used to improve the flow behavior; these substances largely correspond to the carriers described at the beginning. Polyols that come into consideration here preferably have 2 to 15 carbon atoms and at least two hydroxyl groups. The polyols may also contain other functional groups, in particular amino groups, or be modified with nitrogen.

Suitable preservatives include phenoxyethanol, formaldehyde solution, parabens, pentanediol or sorbic acid as well as the silver complexes known as Surfacine® and other classes of substances known and suitable to the skilled person.

Perfume oils are those already defined above. In particular, peppermint oil, spearmint oil, anise oil, star anise oil, caraway oil, eucalyptus oil, fennel oil, lemon oil, wintergreen oil, clove oil, menthol and the like may be considered as aromas.

A preferred embodiment of the cosmetic preparations are toothpastes in the form of an aqueous, pasty dispersion containing polishing agents, humectants, viscosity regulators and optionally other customary components, as well as the mixture of menthofuran and menthol compounds in amounts of 0.5 to 2% by weight.

In mouthwashes, a combination with aqueous-alcoholic solutions of different grades of essential oils, emulsifiers, astringent and tonifying drug extracts, anti-tartaric, antibacterial additives and taste corrigents is readily possible. A further preferred embodiment of the invention is a mouthwash in the form of an aqueous or aqueous-alcoholic solution containing the mixture of menthofuran and menthol compounds in amounts of 0.5 to 2% by weight. In mouthwashes which are diluted before use, sufficient effects can be achieved with higher concentrations according to the intended dilution ratio.

Oral care compositions according to the invention contain, based on the total weight of the composition, preferably 0.1 ppm to 1% by weight, preferably 1 ppm to 0.2% by weight, of at least one active ingredient according to the invention, i.e. a coolant, or an active ingredient mixture, i.e. coolant mixture or flavoring preparation.

The total content of the active ingredient or of the several active ingredients according to the invention or of the coolant mixture or flavoring preparation according to the present invention in ready-to-use mouthwashes is preferably 0.01 to 1% by weight, preferably 0.05 to 0.5% by weight, particularly preferred is a content of 0.1 to 0.3% by weight, in each case based on the total mouthwash.

In mouthwash concentrates, the total content of the active ingredient or of the plurality of active ingredients according to the invention or of the coolant mixture or flavoring preparation according to the present invention is 0.1 to 15% by weight, preferably a content of 0.5 to 8% by weight, particularly preferably 1 to 5% by weight, in each case based on the total mouthwash concentrate.

In toothpastes, the total content of the active ingredient or of the plurality of active ingredients according to the invention or of the coolant mixture or flavoring preparation according to the present invention is 0.1 to 5% by weight, preferably 0.5 to 2% by weight, particularly preferably 0.8 to 1.5% by weight, in each case based on the total toothpaste.

The present invention also includes chewing gum comprising one or more coolants according to the invention or a coolant mixture according to the invention or a flavoring preparation according to the invention.

Chewing gum compositions typically contain a water-insoluble and a water-soluble component. The water-insoluble base, also known as the “gum base”, usually comprises natural or synthetic elastomers, resins, fats and oils, plasticizers, fillers, colorants and optionally waxes. The proportion of the base in the total composition is usually 5 to 95% by weight, preferably 10 to 50% by weight and in particular 20 to 35% by weight. In a typical embodiment of the invention, the base is composed of 20 to 60% by weight of synthetic elastomers, 0 to 30% by weight of natural elastomers, 5 to 55% by weight of plasticizers, 4 to 35% by weight of fillers and, in minor amounts, additives such as colorants, antioxidants and the like, with the proviso that they are water-soluble in small amounts at most.

Suitable synthetic elastomers include, for example, polyisobutylenes with average molecular weights (according to GPC) of 10,000 to 100,000 and preferably 50,000 to 80,000, isobutylene-isoprene copolymers (butyl elastomers), styrene-butadiene copolymers (styrene:butadiene ratio e.g. 1:3 to 3:1), polyvinyl acetates with average molecular weights (according to GPC) of 2,000 to 90,000 and preferably 10,000 to 65,000, polyisoprenes, polyethylene, vinyl acetate-vinyl laurate copolymers and mixtures thereof. Examples of suitable natural elastomers are rubbers such as smoked or liquid latex or guayule as well as natural rubbers such as jelutong, lechi caspi, perillo, sorva, massaranduba balata, massaranduba chocolate, nispero, rosindinba, chicle, gutta hang 1kang and mixtures thereof. The choice of synthetic and natural elastomers and their mixing ratios depends essentially on whether the chewing gums are intended to produce bubbles (“bubble gums”) or not. Elastomer mixtures containing Jelutong, Chicle, Sorva and Massaranduba are preferably used.

Possible fillers or texturizing agents include magnesium or calcium carbonate, ground pumice, silicates, especially magnesium or aluminium silicates, clays, aluminium oxides, talc, titanium dioxide, mono-, di- and tricalcium phosphate and cellulose polymers.

Suitable emulsifiers are tallow, hardened tallow, hardened or partially hardened vegetable oils, cocoa butter, partial glycerides, lecithin, triacetin and saturated or unsaturated fatty acids with 6 to 22 and preferably 12 to 18 carbon atoms and mixtures thereof.

Possible colorants and whitening agents include the FD and C types approved for food coloring, plant and fruit extracts and titanium dioxide.

The base compositions can contain waxes or be wax-free; examples of wax-free compositions can be found in patent specification U.S. Pat. No. 5,286,500, among others.

In addition to the water-insoluble gum base, chewing gum preparations regularly contain a water-soluble portion, which is formed, for example, by softeners, sweeteners, fillers, flavorings, flavor enhancers, emulsifiers, colorants, acidifiers, antioxidants and the like, with the proviso that the ingredients have at least sufficient water solubility. Depending on the water solubility of the specific representatives, individual components can therefore belong to both the water-insoluble and the water-soluble phase. However, it is also possible to use combinations of, for example, a water-soluble and a water-insoluble emulsifier, whereby the individual agents are then in different phases. Typically, the water-insoluble portion makes up 5 to 95% by weight and preferably 20 to 80% by weight of the preparation.

Water-soluble softeners or plasticizers are added to chewing gum compositions to improve chewability and chewing sensation and are typically present in the mixtures in amounts of 0.5 to 15% by weight. Typical examples are glycerol, lecithin and aqueous solutions of sorbitol, hardened starch hydrolysates or corn syrup.

Both sugar-containing and sugar-free compounds are suitable as sweeteners, which are used in amounts of 5 to 95% by weight, preferably 20 to 80% by weight and in particular 30 to 60% by weight based on the chewing gum composition. Typical saccharide sweeteners are sucrose, dextrose, maltose, dextrin, dried invert sugar, fructose, levulose, galactose, corn syrup and mixtures thereof. Sorbitol, mannitol, xylitol, hardened starch hydrolysates, maltitol and mixtures thereof can be used as sugar substitutes. Furthermore, so-called HIAS (“High Intensity Articifical Sweeteners”), such as sucralose, aspartame, acesulfame salts, alitame, saccharin and saccharin salts, cyclamic acid and its salts, glycyrrhizine, dihydrochalcones, thaumatin, monellin and the like alone or in mixtures, can also be considered as additives. The hydrophobic HIAS, which are the subject of the international patent application WO 2002 091849 A1 (Wrigleys), as well as stevia extracts and their active ingredients, in particular ribeaudioside A, are also particularly effective. The amount of these substances used depends primarily on their performance and is typically in the range of 0.02 to 8% by weight.

Fillers such as polydextrose, raftilose, rafitilin, fructooligosaccharides (NutraFlora), palatinose oligosaaccharides, guar gum hydrolysates (Sun Fiber) and dextrins are particularly suitable for the production of low-calorie chewing gum.

The choice of further flavorings is practically unlimited and is not critical to the essence of the invention. Typically, the total amount of all flavorings is 0.1 to 15% by weight and preferably 0.2 to 5% by weight, based on the chewing gum composition. Suitable further flavoring agents are, for example, essential oils, synthetic flavors and the like, such as anise oil, star anise oil, caraway oil, eucalyptus oil, fennel oil, lemon oil, wintergreen oil, clove oil, and the like, as are also used, for example, in oral and dental care compositions.

The chewing gums may also contain auxiliary substances and additives that are suitable, for example, for dental care, especially for combating plaque and gingivitis, such as chlorhexidine, CPC or trichlosan. They may also contain pH regulators (e.g. buffers or urea), anti-caries agents (e.g. phosphates or fluorides), biogenic agents (antibodies, enzymes, caffeine, plant extracts), as long as these substances are approved for use in food and do not interact with each other in an undesirable way.

The present invention also includes cooling patches. Patches according to the invention can be constructed in any desired manner, for example according to the matrix system, the membrane system or the non-woven system.

The plasters according to the invention are manufactured in the usual way.

The matrix system consists of 3 simple parts: the flexible support film, the adhesive matrix containing the active ingredient and a peel-off film. If a non-adhesive matrix is used, adhesive must be applied to one edge of the support film to ensure adhesion to the skin.

A membrane system, on the other hand, has at least 5 parts: a flexible support film, a reservoir with dissolved or suspended active ingredient, a membrane for controlling the release of the active ingredient, an adhesive layer applied to the membrane and a peel-off film.

In the nonwoven system, the layer containing the active ingredient consists of an absorbent nonwoven or porous polymer-impregnated with an active ingredient solution or -suspension. This layer, which is firmly attached to the support film, is covered by a peel-off film. The edge of the support film is provided with adhesive for application to the skin.

In principle, all active ingredients according to the invention can be formulated in this way. The excipients to be used are the usual ones for the production of plasters. In addition to the adhesive agent, usually a polymer with a glass transition temperature between −70 and −10° C., in particular −55 and −25° C., and a carrier film coated with this adhesive agent, and the active ingredient, emulsifiers, thickening agents and substances intended to influence the release of the active ingredient and other auxiliaries are often added.

The sticky polymers with the low glass temperatures mentioned above are known. The self-adhesive tapes and films are intended to adhere to the human skin on mere contact, but the cohesion of the adhesive layer and its adhesion to the carrier film should be greater than the adhesion to the skin, so that they can be removed again largely without leaving any residue. These are usually copolymers based on acrylic and methacrylic acid esters of alcohols with 2 to 12, in particular 4 to 8 carbon atoms, which can contain numerous other comonomers polymerized in, for example (meth)acrylic acid, (meth)acrylonitrile, (meth)acrylamide, N-tert.butyl (meth)acrylamide, vinyl esters such as vinyl acetate, vinyl propionate or vinyl butyrate, other vinyl compounds such as styrene, and butadiene. Butyl acrylate and 2-ethylhexyl acrylate are particularly noteworthy. The polymers can be crosslinked by adding small amounts of comonomers with 2 or more copolymerizable double bonds, for example diacrylates, such as butanediol diacrylate, or divinyl compounds, such as divinylbenzene, or by adding other crosslinking agents, e.g. melamine-formaldehyde resins. Polyisobutylenes and polyvinyl ethers of different molecular weights can also be used as tacky polymers.

The particle size of the dispersions should be between 50 and 500 nm, in particular between 50 and 200 nm. The particle size and the degree of crosslinking can be adjusted in a known manner depending on the polymerization conditions and the comonomers. Smaller particle sizes and a higher degree of crosslinking can increase the release of the active ingredient.

Matrix patches can be produced in the usual way by dissolving or finely dispersing the active ingredient in a suitable polymer solution and then drawing out this active ingredient-containing self-adhesive mass into a film using a roller or doctor blade application process. In some cases, it is advisable to dissolve or finely disperse the active ingredient in an organic solvent, e.g. ethanol or acetone, before adding it to the polymer solution. This can achieve a better distribution of the active ingredient in the polymer.

The patches can also be produced by incorporating the active ingredient in fine powder form (particle size below 200 μm, in particular below 50 μm) into the aqueous latex dispersion, or by dispersing or dissolving it in an aqueous emulsifier solution and mixing this mixture into the aqueous latex dispersion at a temperature of 10 to 80, in particular 30 to 70° C. In addition, the salt of an active ingredient in aqueous solution can also be mixed with the polymer dispersion at a pH value at which the active ingredient is predominantly present in the water-soluble ionized form. By shifting the pH, the active ingredient is then converted into the uncharged, water-insoluble form and simultaneously emulsified into the dispersion.

The active ingredient is conveniently prepared, the emulsifier and water are added and then mixed with the polymer dispersion. The dispersion containing the active ingredient obtained in this way may be provided with further additives and, as mentioned, is drawn out to form a film on a supporting film and dried in a manner known per se. The drying temperature can be between room temperature and 100° C., whereby an optimum between the desired rapid drying and the avoidance of bubble formation in the film as well as thermal stress on the active ingredient is generally 35 to 45° C. This process has the great advantage of avoiding organic solvents. However, in principle, all other conventional manufacturing processes for matrix patches can also be considered.

The resulting films have thicknesses of 10 to 800 μm, preferably 50 to 300 μm. The film can be produced continuously or discontinuously. The application process can be repeated several times until the film has reached the desired thickness. The sticky polymer layer contains the active ingredient in a concentration ranging from 1 to 40% by weight, in particular 5 to 25% by weight. The same concentration also applies to the reservoir liquid in the membrane system and to the active ingredient solution or dispersion with which the nonwoven or porous polymer is impregnated in the nonwoven system.

As emulsifiers for both the active ingredients according to the invention, i.e. the coolant according to the invention or the coolant mixture according to the invention or the flavoring preparation according to the invention as well as the polymers, the surfactants commonly used for this purpose are used, such as the sodium salt of longer-chain fatty acids and the sulfuric acid half ester of a (optionally oxethylated) fatty alcohol as examples of anionic surfactants as well as polyoxethylated alkylphenols and longer-chain fatty alcohols (e.g. hexadecane-(I)-ol and glycerol fatty acid partial esters as examples of nonionic surfactants and coemulsifiers.

The desired viscosity of the ready-to-extract mass can be adjusted using polyacrylic acids or cellulose derivatives, for example. Melamine-formaldehyde resins, for example, can be used as additional cross-linking agents to improve the cohesion and thus the adhesive properties of the films.

Swelling agents such as polyvinylpyrrolidone, cellulose derivatives or polyacrylates have the effect of improving the release of active ingredients, as the film can absorb more water, thereby reducing the diffusion resistance. The release of the active ingredients can also be improved by adding hydrophilic plasticizers such as glycerine, 1,2-propanediol of polyethylene glycols and lipophilic plasticizers such as triacetin, dibutyl phthalate or isopropyl myristate.

Matrix patches usually result in a 1st order release of the active ingredient. The use of fillers that adsorb the active ingredient, such as aerosil, microcrystalline cellulose or lactose, results in an approximate 0th order release.

The support film onto which the self-adhesive composition containing the active ingredient is dried is practically impermeable to both the active ingredient and water vapor. It can, for example, consist of an aluminium-plastic composite film, a metallized plastic film, a plastic film which is provided with a barrier layer of e.g. polyvinylidene chloride on the active ingredient side, or a simple plastic film, e.g. polyester film.

The patches according to the invention, which are constructed according to the membrane system, are also produced in the usual way. The plasters constructed according to the nonwoven system are produced by soaking nonwovens or porous polymers attached to the support film with a solution or dispersion of the active ingredient in a hydrophilic or lipophilic solvent or solvent mixture. The impermeable peel-off film is then applied.

In principle, the active ingredient content in the preparations according to the invention can vary over a wide range, such as 0.1 ppm to 10% by weight, preferably 1 ppm to 10% by weight.

The present invention also relates to textile products which are equipped with a coolant according to the invention or a coolant mixture according to the invention.

Finishing textiles with coolants with a cooling effect is used in particular where items of clothing can come into direct contact with the skin so that the active ingredient can exert its effects, e.g. locally or systemically, through transdermal transfer. Recently, there have been reports of textiles that are equipped with so-called wellness additives, i.e. substances that promote well-being.

An insecticidal finish, on the other hand, is of interest with regard to material protection, e.g. finishing the textile against moth damage, etc., but also in particular for repelling parasitic insects such as mosquitoes.

The fundamental problem when finishing textiles with active ingredients is the bonding of the active ingredient to the textile carrier, which on the one hand must ensure the permanence of the finish and on the other hand must be selected in such a way that the active ingredient does not lose its effect. Various approaches have been proposed in the state of the art.

Cyclodextrins, for example, have been proposed for binding active ingredients to textiles. Cyclodextrins are cyclic oligosaccharides that are formed by the enzymatic degradation of starch. The most common cyclodextrins are α-, β and γ-cyclodextrins, which consist of six, seven or eight α-1,4-linked glucose units. A characteristic property of cyclodextrin molecules is their ring structure with largely invariable dimensions. The inner diameter of the rings is about 570 pm for α-cyclodextrin, about 780 pm for β-cyclodextrin and about 950 pm for γ-cyclodextrin. Due to their structure, cyclodextrins are able to entrap guest molecules, especially hydrophobic guest molecules, in varying amounts until saturation.

The state of the art describes the finishing of textiles with fragrances and other low-molecular organic active ingredients which are bound to the textile via an amylose-containing substance with an amylose content of at least 30%. Due to the amylose content of the amylose-containing substance, the active ingredient is bound to the textile and released in a controlled manner so that the effect is maintained over a long period of time. It is assumed that, similar to cyclodextrins, the active ingredient is reversibly bound in the cavities formed by the helical conformation of the amylose in the sense of an inclusion compound, which on the one hand fixes the active ingredient to the surface of the textile carrier and on the other hand enables controlled release.

In addition to amylose, all substances, in particular amylose-containing starches, i.e. native starches, modified starches and starch derivatives, whose amylose content is at least 30% by weight and in particular at least 40% by weight, are suitable for finishing textiles in accordance with the invention. The starch can be native, e.g. maize starch, wheat starch, potato starch, sorghum starch, rice starch or maranta starch, obtained by partial digestion of native starch or chemically modified. Pure amylose as such is also suitable, e.g. enzymatically obtained amylose, e.g. amylose obtained from sucrose. Mixtures of amylose and starch are also suitable, provided that the total content of amylose is at least 30% by weight, based on the total weight of the mixture. It is understood that here and in the following all indications in % by weight which refer to amylose or amylose-containing substances always refer to the total weight of amylose+starch in the case of mixtures of amylose and starch, unless expressly indicated otherwise. Particularly suitable according to the invention are amylose-containing substances, in particular amylose and amylose-containing starches as well as amylose/starch mixtures, the amylose content of which is at least 40% by weight and in particular at least 45% by weight, based on the total weight of the substance. As a rule, the amylose content will not exceed 90% by weight and in particular 80% by weight. Such substances are known and commercially available. For example, amylose-containing starches are marketed by the companies Cerestar under the trade name Amylogel® and National Starch under the trade names HYLON® V and VII.

In order to achieve the binding of the active ingredient(s) to the textile, the textile can be finished with the amylose-containing substance generally in an amount of at least 0.5% by weight, preferably at least 1% by weight and in particular at least 2% by weight, in each case based on the weight of the textile. As a rule, the amylose-containing substance is used in an amount of not more than 25% by weight, often not more than 20% by weight and in particular not more than 15% by weight, based on the weight of the textile, so as not to adversely affect the tactile properties of the textile. First, the textile material is finished with the amylose-containing substance as such and then the textile thus finished is treated with a suitable preparation of the active substance. In this way, the amylose-containing substance on the textile material is loaded with the active ingredient. However, the amylose-containing substance can also be used together with an active ingredient to finish the textile. In this case, the active ingredient and the amylose-containing substance can be used both as a mixture of separate components and in the ready-made form of the amylose-active ingredient complex. As a rule, the active ingredient is used in an amount that is sufficient for the desired effect. The upper limit is determined by the maximum absorption capacity of the amylose units of the amylose-containing substance used and will generally not exceed 20% by weight and often 10% by weight, based on the amylose content of the substance. If desired, the active ingredient is generally used in an amount of 0.00001 to 15% by weight, 0.0001 to 10% by weight, 0.001 to 5% by weight, 0.005 to 1% by weight or 0.1 to 10% by weight or 0.5 to 5% by weight, based on the amylose content of the amylose-containing substance.

Combinations of active substances according to the invention with other active substances known per se and suitable for textile finishing can also be used for textile finishing.

In principle, all organic compounds and mixtures of organic compounds that are known as active substances and that induce a physiological effect in living organisms such as humans and animals, including microorganisms, are suitable as further active substances. These include active substances that are known to form inclusion compounds with cyclodextrins. Particularly suitable are active substances which have hydrocarbon groups and in particular aliphatic, cycloaliphatic and/or aromatic structures. The molecular weight of the active ingredients is typically below 1000 Daltons and often in the range of 100 to 600 Daltons. Inorganic compounds such as hydrogen peroxide, which are known to be able to be bound in cyclodextrins, are also suitable.

Other active ingredients include, in particular, pharmaceutical active ingredients and active ingredients that promote the well-being of living beings, especially humans, and which are also commonly referred to as “wellness additives”. Unlike pharmaceutical active ingredients, wellness additives do not necessarily have to have a therapeutic effect. Rather, the effect promoting well-being can be based on a variety of factors such as caring, stimulating, cosmetic or other effects. Equally suitable are organic active ingredients that act against parasitic organisms. These include, for example, active ingredients that act against fungi and/or microorganisms, e.g. fungicides and bactericides, or that act against animal pests such as snails, worms, mites, insects and/or rodents, e.g. nematicides, molluscicides, insecticides, acaricides, rodenticides and repellent active ingredients, as well as active ingredients against weeds, i.e. herbicides, or fragrances.

Preferred active pharmaceutical ingredients are those that are known to be absorbed through the skin. These include, for example, ibuprofen, flurbiprofen, acetylsalicylic acid, acetamidophen, apomorphine, butylated hydroxytoluene, chamzulene, gujazulene, chlorthalidone, cholecalciferol, dicumarol, digoxin, diphenylhydantoin, furosemide, hydroflumethiazide, indomethacin, iproniazid phosphate, nitroglycerin, nicotine, nicotinic acid amide, oubain, oxprenolol, papaverine alkaloids such as papaverine, laudanosine, ethaverine and narcotine as well as berberine, retionol, trans-retinoic acid, pretinol, spironolactone, sulpiride, theophylline, theobromine, corticosteroids and derivatives such as testosterone, 17-methyltestosterone, cortisone, corticosterone, dexamethasone, triamcinolone, methylprednisolone, fludrocortisone, fluocortolone, prednisone, prednisolone, progesterone, et al. a. estrogens and gestagens such as estradiol, estriol, ethinylestradiol-3-methylether, norethisterone and ethisterone, as well as phenethylamine and derivatives such as tyramine, adrenaline, noradrenaline and dopamine. Examples of active substances suitable according to the invention with an effect against parasitic organisms are, for example, nematicides, bactericides, fungicides, insecticides, insect repellents, acaricides and molluscicides. Examples of bactericidal and fungicidal substances include:
Antibiotioca, e.g. cycloheximide, griseofulvin, kasugamycin, natamycin, polyoxin, streptomycin, penicillin or gentamycin;
Organic compounds and complexes of biocidal metals, e.g. Complexes of silver, copper, tin and/or zinc such as bis-(tributyltin) oxide, copper, zinc and tin naphthenates, oxine-copper such as Cu-8, tris-N-(cyclohexyldiazeniumdioxy)-aluminum, N-(cyclohexyldiazeniumdioxy)-tributyltin, bis-N-(cyclohexyldiazeniumdioxy)-copper;
Quaternary ammonium salts, e.g. benzyl-Cs- to cis-alkyldimethylammonium halides, in particular chlorides (benzalkonium chlorides);
Aliphatic nitrogen fungicides and bactericides such as Cymoxanil, Dodin, Dodicin, Guazidine, Iminocta-din, Dodemorph, Fenpropimorph, Fenpropidin, Tridemorph;
Substances with peroxide groups such as hydrogen peroxide, and organic peroxides such as dibenzoyl perodide;
Organic chlorine compounds such as chlorhexidine;
Triazole fungicides such as azaconazole, cyproconazole, diclobutrazole, difenoconazole, diniconazole, epoxiconazole, fenbuconazole, fluquinconazole, flusilazole, flutriafol,
hexaconazole, metconazole, propiconazole, tetraconazole, tebuconazole and triticonazole; strobilurins such as dimoxystrobin, fluoxastrobin, kresoxim-methyl, metominostrobin, orysastrobin, picoxystrobin, pyraclostrobin and trifloxystrobin; Sulfonamides such as tolylfluanid and diclofluanid;
iodine compounds such as diiodomethyl-p-tolyl sulfone, napcocide 3-lod-2-propynyl alcohol, 4-chlorophenyl-3-iodopropyl formal, 3-bromo-2,3-diiodo-3-propenylethyl carbonate, 2,3,3-triiodoallyl alcohol, 3-lod-2-propynyl-n-hexyl carbamate, 3-bromo-2,3-diiodo-2-propenyl alcohol, 3-lod-2-propinyl phenyl carbamate, 3-lod-2-propinyl n-butyl carbamate, 0-1-(6-lod-3-oxohex-5-inyl)phenyl carbamate, 0-1-(6-lod-3-oxohex-5-inyl)butyl carbamate;
Isothiazolinones such as N-methylisothiazolin-3-one, 5-chloro-N-methylisothiazolin-3-one, 4,5-dichloro-N-octylisothiazolin-3-on, 1,2-benzisothiazol-3(2H)on, 4,5-trimethylisothiazol-3-one and N-octyl-isothiazolin-3-one.

Examples of insecticides and acaricides are: Organophosphates such as acephate, azamethiphos, azinphos-methyl, chlorpyrifos, chlorpyriphos-methyl, chlorfenvinphos, diazinon, dichlorvos, dicrotophos, dimethoate, disulfoton, ethion, fenitrothion, fenthion, isoxathion, malathion, methamidophos, Methidathion, Methyl-Parathion, Mevinphos, Monocrotophos, Oxydemeton-methyl, Paraoxon, Parathion, Phenthoate, Phosalone, Phosmet, Phosphamidon, Phorate, Phoxim, Pirimiphos-methyl, Profenofos, Prothiofos, Sulprophos, Triazophos, Trichlorfon; in particular pyrethroids such as acrinatrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, α-cypermethrin, β-cypermethrin, A-cypermethrin, zeta-cypermethrin, cyphenothrin, deltamethrin, dimefluthrin, dimethrin, empenthrin, fenfluthrin, fenprithrin, fenpropathrin, fenvalerate, esfenvalerate, flucythrinate, fluvinate, tau-fluvinate, furethrin, permethrin, biopermethrin, trans-permethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute and silafulfen; Pyrrole and pyrazole insecticides such as acetoprole, ethiprole, fipronil, tebufenpyrad, tolfenpyrad, chlorfenapyr and vaniliprole.

Examples of repellent active ingredients are in particular anthraquinone, acridine bases, copper naphthenate, butopyronoxyl, dibutyl phthalate, dimethyl phthalate, dimethyl carbate, ethohexadiol, hexamides, metho-quin-butyl, N-methylneodecanamide, camphor, bergamot oil, pyrethrum, clove oil, geranium oil, thyme oil and in particular diethyl-m-toluamide and 1-piperidinecarboxylic acid-2-(2-hydroxyethyl)-1-methylpropyl ester (picardin). Examples of wellness additives include in particular the substances and substance mixtures listed below, e.g. fats, preferably of vegetable origin, e.g. lecithins, vegetable oils such as jojoba oil, tea tree oil, clove oil, evening primrose oil, almond oil, coconut oil, avocado oil, soybean oil and the like, fatty acids, e.g. w-6 fatty acids, linolenic acid, linoleic acid, waxes of animal or vegetable origin such as beeswax, candelilla wax, shea butter, shorea butter, mango kernel butter, Japan wax and the like, vitamins, in particular fat-soluble vitamins, e.g. tocopherols, vitamins of animal or vegetable origin such as beeswax, candelilla wax, shea butter, shorea butter, mango kernel butter, Japan wax and the like, e.g. tocopherols, vitamin E, vitamin A and the like, cortico-steroids such as cortisone, corticosterone, dexamethasone, triamcinolone, methylprednisolone, fludrocortisone, fluocortolone, prednisone, prednisolone, progesterone, amino acids, e.g. arginine, methionine; plant extracts such as algae extract, horse chestnut extract, mango extract and the like.

To improve the wash permanence of the finish according to the invention, it has proved useful to fix the amylose-containing substance to the textile with a binder. Suitable binders include film-forming, water-insoluble polymers and low-molecular reactive substances that polymerize when heated. As a rule, the binder is used in an amount such that the weight ratio of amylose-containing substance to water-insoluble polymer is in the range from 1:1 to 100:1, preferably in the range from 1.5:1 to 50:1 and in particular in the range from 2:1 to 20:1.

As a rule, the film-forming polymers are used in the form of an aqueous dispersion of finely divided polymer particles. The particle size is of secondary importance for the success of the invention. However, it is generally below 5 pm (weight average) and is usually 50 nm to 2 μm.

In particular, the film-forming polymer can have a glass transition temperature TG in the range from −40 to 100° C., preferably −30 to +60° C., in particular −20 to +40° C. If the polymeric binder comprises several polymer components, at least the main component should have a glass transition temperature in this range. In particular, the glass transition temperature of the main component is in the range from −30° C. to +60° C. and especially preferably in the range from −20° C. to +40° C. Preferably, all polymer components have a glass transition temperature in these ranges. The specified glass transition temperatures refer to the “midpoint temperature” determined according to ASTM-D 3418-82 using DSC. In the case of crosslinkable binders, the glass transition temperature refers to the non-crosslinked state.

Examples of suitable film-forming polymers are based on the following polymer classes:
(1) Polyurethane resins;
(2) Acrylate resins (pure acrylates: copolymers of alkyl acrylates and alkyl methacrylates);
(3) Styrene acrylates (copolymers of styrene and alkyl acrylates); (4) Styrene/butadiene copolymers;
(5) Polyvinyl esters, in particular polyvinyl acetates and copolymers of vinyl acetate with vinyl propionate;
(6) Vinyl ester olefin copolymers, e.g. vinyl acetate/ethylene copolymers;
(7) Vinyl ester acrylate copolymers, e.g. vinyl acetate/alkyl acrylate copolymers and vinyl acetate/alkyl acrylate ethylene terpolymers.

Such polymers are known and commercially available, e.g. polymers of classes (2) to (7) in the form of aqueous dispersions under the names ACRONAL, STYROFAN, BUTOFAN (BASF-AG), MOWILITH, MOWIPLUS, APPRETAN (Clariant), VINNAPAS, VINNOL (WACKER). Aqueous polyurethane dispersions (1) suitable for the process according to the invention are in particular those used for coating textiles. Suitable substances are well known to the skilled person. Aqueous polyurethane dispersions are commercially available, for example, under the trade names Alberdingk® from Alberdingk, Impranil® from BAYER AG, Permutex® from Stahl, Waalwijk, Netherlands, from BASF SE or can be produced according to known processes, such as those described in the relevant technical literature. The film-forming polymers can be self-crosslinking, i.e. the polymers have functional groups (crosslinkable groups) which react with each other, with the functional groups of the amylose or with a low-molecular crosslinking agent to form bonds when the composition is dried or heated. Examples of crosslinkable functional groups include aliphatically bonded OH groups, NH—CH₂—OH groups, carboxylate groups, anhydride groups, capped isocyanate groups and amino groups. A polymer that still has free OH groups as reactive groups is often used. As a rule, the proportion of reactive functional groups is 0.1 to 3 mol/kg polymer. Crosslinking can be achieved within the polymer by reaction of complementary reactive functional groups. Preferably, crosslinking of the polymer is achieved by adding a crosslinker which has reactive groups which are complementary to the functional groups of the crosslinker in terms of their reactivity. Suitable pairs of functional groups which have a complementary reactivity are known to the skilled person. Examples of such pairs are OH/COOH, OH/NCO, NH₂/COOH, NH₂/NCO and M²⁺/COOH, where M²⁺ stands for a divalent metal ion such as Zn²⁺, Ca²⁺, or Mg²⁺. Examples of suitable crosslinkers are the di- or polyols mentioned below for the polyurethanes; primary or secondary diamines, preferably primary diamines, e.g. alkylenediamines such as hexamethylenediamine, diethylenetriamine, triethylenetetramine, tetraethylenepentamine, N,N-bis[(aminopropyl)amino]-ethane, 3,6-dioxaoctanediamine, 3,7-dioxanonanediamine, 3,6,9-trioxaundecanediamine or Jeffamine, (4,4-diaminodicyclohexyl)methane, (4,4′-diamino-3,3-dimethyldicyclohexyl)methane; Amino alcohols such as ethanolamine, hydroxypropylamine; ethoxylated di- and oligoamines; dihydrazides of aliphatic or aromatic dicarboxylic acids such as adipic acid dihydrazide; dialdehydes such as glyoxal; partially or completely O-methylated melamines, as well as compounds or oligomers which contain on average two or more, preferably three or more isocyanate groups or reversibly e.g. hydrogen sulfite blocked isocyanate groups. In this case, the quantitative ratio of crosslinkerto polymeric binder is calculated such that the molar ratio of the reactive groups in the polymeric binder (total amount of reactive groups in the polymers) to the reactive groups in the crosslinker is usually in the range from 1:10 to 10:1 and preferably in the range from 3:1 to 1:3. Typically, the weight ratio of polymeric binder (calculated as a solid) to crosslinker is in the range of 100:1 to 1:1 and in particular in the range of 50:1 to 5:1.

As an alternative to fixing the amylose-containing substance with water-insoluble polymers, the amylose or the amylose-containing substance can also be fixed to the textile material with reactive compounds which have at least one group which is reactive towards the OH groups of the amylose and at least one further functional group which is reactive towards the functional groups on the fibers of the textile material, e.g. OH groups, NH₂ groups or COOH groups. The reactive compounds include the crosslinkers mentioned above as well as the substances proposed in DE 40 35 378 A for fixing cyclodextrins, e.g. N-hydroxymethyl and N-alkoxymethyl derivatives of urea or urea-like compounds such as dimethylolurea (bis(hydroxymethyl)urea), di(methoxymethyl)urea, dimethylolalkanediol diurethanes such as N,N-dimethylolethylene urea (N,N-bis(hydroxymethyl)imidazolin-2-one), N,N-dimethylol-dihydroxyethylene urea (N,N-bis(hydroxymethyl)-4,5-dihydroxyimidazolin-2-one), dimethylolpropy-lene urea and the like. Such materials are commercially available in the form of aqueous formulations for finishing textiles, e.g. under the trade names Fixapret® and Fixapret®-eco from BASF SE. The reactive materials that can be used to fix the amylose-containing substance to the textile material include, in particular, compounds with 2, 3, 4 or more (possibly reversibly blocked) isocyanate groups, especially the polyisocyanate prepolymers based on polyether and polyester urethanes that are reversibly blocked with bisulphite or CH-acid compounds or oximes, e.g. butanone oxime, which are described in DE 2837851, DE 19919816 and the earlier patent application EP 03015121. Such products are also commercially available, for example under the trade names PROTOLAN® 367 and PROTOLAN® 357 from Rotta GmbH, Mannheim.

To fix the amylose-containing substance, the procedure known for the fixation of cyclodextrins can also be used in an analogous manner, in which the cyclodextrin or, in the present case, the amylose-containing substance is provided with reactive anchors, for example, by mixing it with dicarboxylic acids or dicarboxylic acid anhydrides such as maleic acid, fumaric acid, maleic anhydride, succinic acid, succinic anhydride or adipic acid, with diisocyanates, e.g. toluene diisocyanate, isophorone diisocyanate, tetramethylene diisocyanate or hexamethylene diisocyanate, or with aminocarboxylic acids such as toluene diisocyanate, isophorone diisocyanate, tetramethylene diisocyanate or hexamethylene diisocyanate. e.g. toluene diisocyanate, isophorone diisocyanate, tetramethylene diisocyanate or hexamethylene diisocyanate, or with aminocarboxylic acids in a manner known per se such that only one of the functionalities present in these compounds reacts with the OH groups of the amylose-containing substance and the other is retained for bonding to the reactive groups of the fiber material. Reactive anchors can also be produced on the amylose-containing substance by reaction with 1,3,5-trichlorotriazine, 2,3-dichloroquinoxaline-5,6-carboxylic acid chloride and chlorodifluoropyrimidine. Furthermore, alkoxysilanes such as diethoxydimethylsilane, dimethoxydimethylsilane, triethoxyphenylsilane, tetraethoxysilane and dimeric, trimeric and higher condensation products of these compounds can also be used to fix the amylose.

In principle, all textile materials can be finished in this way, i.e. non-manufactured goods as well as manufactured goods. Textile materials here and in the following include woven, knitted, knitted and non-woven fabrics. The textile materials can be composed of natural fiber yarns, synthetic fiber yarns and/or blended yarns. In principle, all fiber materials commonly used for the production of textiles can be considered as fiber materials. These include cotton, wool, hemp fibers, sisal fibers, flax, ramie, polyacrylonitrile fibers, polyester fibers, polyamide fibers, viscose fibers, silk, acetate fibers, triacetate fibers, aramid fibers and the like, as well as mixtures of these fiber materials.

The textile materials can be finished or treated with the amylose-containing substance in a manner known per se, e.g. by means of processes described for finishing textiles with cyclodextrins.

Examples include processes in which the amylose-containing substance, possibly as a complex with the active ingredient, is already spun into the fiber, filament and/or yarn from which the fabric is produced.

However, the textile material is often treated with the amylose-containing substance or a complex of amylose-containing substance and active ingredient before or after finishing. As a rule, the textile is treated with an aqueous liquor containing sufficient quantities of the amylose-containing substance and, if necessary, the active ingredient. Depending on the type of application and the desired quantity in which the amylose-containing substance is to be applied, the concentration of amylose-containing substance in the liquor is in the range from 1 to 40% by weight, in particular in the range from 2 to 20% by weight and especially in the range from 4 to 15% by weight.

The type of treatment is of secondary importance and can, for example, be applied as a minimum application, e.g. by spraying, as a normal application in a padder or as a high-moisture application. In this case, the textile material is soaked with the aqueous liquor. If necessary, excess liquor can then be removed, e.g. by squeezing to a liquor absorption of approx. 30 to 120%. Another possibility for treating the textile with an amylose-containing substance or a complex of amylose-containing substance and active ingredient is to prepare a liquor with water containing the desired amount of amylose-containing substance and, if necessary, active ingredient, e.g. 0.5 to 20% by weight (based on the mass of the textile to be finished). The textile material is soaked with the treatment liquor in suitable finishing units (e.g. reel skid; roller skid; paddle; etc.) for a certain period of time, e.g. 10 to 60 minutes, and then squeezed and/or spun off as described above. The liquor ratio is generally in the range of 1 2 to 1:50 and in particular in the range of 1:3 to 1:20.

Such processes are known to the skilled person from the relevant technical literature.

As a rule, the treatment with the liquor is followed by a drying process. The temperatures are usually in the range of 100 to 200° C. and preferably in the range of 120 to 180° C. Drying can be carried out in the usual devices for this purpose, in the case of finished goods, for example, by drying at the above-mentioned temperatures. In the case of non-manufactured goods, the textile material is usually passed over one or more stenter frames after application.

If the amylose-containing substance is used together with a film-forming polymer, drying leads to a fixation of the amylose-containing substance on the textile fibers. As a rule, the drying temperature will then not fall below 100° C. and is preferably in the range from 120 to 200° C. and in particular in the range from 140 to 180° C. Drying generally takes place over a period of 1 to 10 minutes, in particular 1 to 2 minutes, although longer drying times are also suitable. For treatment with an aqueous liquor, it has been found to be advantageous if the aqueous liquor contains, in addition to the amylose-containing substance and possibly the active substance, at least one surface-active substance (or surface-active substance) which is suitable for dispersing the amylose-containing substance and the active substance in the aqueous liquor. Preferably, the surfactant is an oligomeric or polymeric dispersant. In contrast to low molecular weight surface-active substances, the term oligomeric or polymeric dispersant comprises such dispersants whose number-average molecular weight is generally at least 2000 daltons, e.g. 2000 to about 100000 daltons and in particular is in the range of about 3000 to 70000 daltons. As a rule, the aqueous liquor contains the polymeric or oligomeric dispersant in an amount of from 0.5 to 20% by weight, preferably from 1 to 18% by weight and in particular from 5 to 15% by weight, based on the amylose-containing substance.

Suitable oligomeric or polymeric dispersants are soluble in water and include both neutral and amphoteric water-soluble polymers as well as cationic and anionic polymers, the latter being preferred. Examples of neutral polymeric dispersants are polyethylene oxide, ethylene oxide/propylene oxide copolymers, preferably block copolymers, polyvinylpyrrolidone and copolymers of vinyl acetate with vinylpyrrolidone.

The preferred anionic oligomeric or polymeric dispersants are characterized by the fact that they have carboxyl groups and/or sulfonic acid groups and are usually used as salts, e.g. as alkali metal salts or ammonium salts. Preferred anionic dispersing agents are, for example, carboxylated derivatives of cellulose such as carboxymethyl cellulose, homopolymers of ethylenically unsaturated C3 to C8 mono- and C4 to C8 dicarboxylic acids, e.g. acrylic acid, methacrylic acid, maleic acid, itaconic acid, copolymers of at least two different ethylenically unsaturated C3- to C8-mono- and C4- to C8-dicarboxylic acids as mentioned above, and copolymers of at least one of the aforementioned ethylenically unsaturated C3- to C8-mono- or C4- to C8-dicarboxylic acids with at least one neutral comonomer. Examples of neutral comonomers are N-vinyllactams such as N-vinylpyrrolidone, vinyl esters of aliphatic C2- to C16-carboxylic acids such as vinyl acetate, vinyl propionate, amides of the above-mentioned ethylenically unsaturated carboxylic acids, such as acrylamide, methacrylamide and the like, hydroxy-C1 to C4 alkyl (meth)acrylates such as hydroxyethyl acrylate and methacrylate, esters of ethylenically unsaturated C3 to C8 mono- or C4 to C8 dicarboxylic acids with polyethers, e.g. esters of acrylic acids with polyethers. e.g. esters of acrylic acid or methacrylic acid with polyethylene oxides or ethylene oxide/propylene oxide block copolymers, vinyl aromatics such as styrene and C2 to C16 olefins such as ethylene, propene, 1-hexene, 1-octene, 1-decene, 1-dodecene and the like. Homopolymers of ethylenically unsaturated sulfonic acids such as styrene sulfonic acid and acrylamidopropane sulfonic acid and their copolymers with the aforementioned comonomers are also preferred. In the copolymers, the proportion of ethylenically unsaturated acid will generally be at least 20% by weight and will not exceed a value of 90% by weight and in particular 80% by weight, in each case based on the total weight of all the monomers constituting the polymer. Copolymers of at least one of the above-mentioned acids and at least one comonomer are known for this purpose and are commercially available, for example the copolymers of acrylic acid and maleic acid as Sokalan brands of BASF SE.

Other preferred anionic dispersants are phenolsulfonic acid-formaldehyde condensates and naphthalenesulfonic acid-formaldehyde condensates (e.g. BASF's Tamol and Setamol brands) and lignosulfonates.

Suitable dispersing agents are also low-molecular anionic, non-ionic, cationic, ampholytic and zwitterionic surfactants. Suitable surfactants are, for example the alkali metal, ammonium or amine salts of C8 to C18 alkyl sulphates, such as sodium lauryl sulphate; C8 to C18 alkyl sulphonates, such as dodecyl sulphonate; C8 to C18 alkyl ether sulphates; and C8 to C18 alkyl ethoxylates; polyoxyethylene sorbitan esters; C8 to C18 alkyl glycinates; C8 to C18 alkyl dimethylamine oxides; betaines, etc. Preferred are the alkyl sulfates and alkyl sulfonates.

If the amylose-containing substance is not used together with a film-forming, water-insoluble polymer, the textile can be treated with the polymer in a separate step. In particular, the treatment is carried out together with the amylose-containing substance. Accordingly, a particular embodiment relates to a process in which the aqueous liquor additionally comprises a dispersed, film-forming, water-insoluble polymer of the type described above. The amount of film-forming polymer is selected such that the weight ratio of amylose-containing substance to water-insoluble polymer is in the range from 1:1 to 100:1, preferably in the range from 1.5:1 to 50:1 and in particular in the range from 2:1 to 20:1.

The finishing of the textile with the coolant according to the invention or the coolant mixture according to the invention can be carried out in a separate operation or in one operation together with the finishing with the amylose-containing substance.

If the textile is treated with the active ingredient in a separate work step, the textile is also treated with an aqueous liquor of the active ingredient. For this purpose, the active ingredient, which is usually not soluble in water, is usually emulsified or dispersed in water, if necessary using suitable surface-active substances. Suitable surface-active substances are in particular the low-molecular surfactants mentioned above and preferably the non-ionic surfactants, in particular polyoxyethylene sorbitan esters, esters of mono- or oligosaccharides with C6 to C18 fatty acids and particularly preferably C8 to C18 alkyl ethoxylates, especially those with a degree of ethoxylation in the range from 6 to 50.

As a rule, the aqueous liquor contains the active substance in an amount of 0.1 to 10% by weight and in particular in an amount of 0.2 to 5% by weight. The amount of surface-active substance is generally in the range of 0.5 to 50% by weight and in particular in the range of 3 to 30% by weight, based on the active substance. The active ingredient can be applied from an aqueous liquor using the usual methods, e.g. by means of a foulard. However, it is also possible to apply the active ingredient and the amylose-containing substance in a single operation. In this case, it is possible to proceed as described for finishing with the amylose-containing substance, whereby the aqueous liquor of the amylose-containing substance now also contains at least one active ingredient. The active ingredient can be added separately to the liquor or in the form of an inclusion compound, i.e. in the form of a host-guest complex with the amylose-containing substance.

The coolants or coolant mixtures according to the present invention can be used to finish any textiles, i.e. non-manufactured goods as well as manufactured goods. Textile materials here and hereinafter include woven fabrics, knitted fabrics, knitted fabrics and non-woven fabrics. The textile materials can be composed of natural fiber yarns, synthetic fiber yarns and/or blended yarns. In principle, all fiber materials commonly used for the production of textiles can be considered as fiber materials. These include cotton, wool, hemp fibers, sisal fibers, flax, ramie, polyacrylonitrile fibers, polyester fibers, polyamide fibers, viscose fibers, silk, acetate fibers, triacetate fibers, aramid fibers and the like, as well as mixtures of these fiber materials. Glass fibers and mixtures of the aforementioned fiber materials with glass fibers, e.g. glass fiber/Kevlar mixtures, are also suitable. The type of textile material depends primarily on the desired application. The textiles to be finished can be ready-made products such as clothing, including underwear and outerwear, e.g. shirts, pants, jackets, outdoor, trekking and military equipment, roofs, tents, nets, e.g. insect nets and curtains, hand and bath towels, bed linen and the like. In the same way, the finishing can be done on the raw material in bale or roll form.

With an amylose-based active ingredient finish, the active ingredients remain in the finished textiles even after several washes. In addition, the textiles finished in this way are characterized by a pleasant feel, which is particularly beneficial for the wearing comfort of clothing made from these textiles.

In addition to protecting humans, textiles containing active substances against parasitic organisms such as insects and acarids are also particularly suitable for protecting animals against ticks, mites, fleas and the like.

The present invention also relates to cooling tobacco products.

The active ingredients according to the invention, i.e. the coolant according to the invention or the coolant mixture according to the invention or the flavoring preparation according to the invention, can advantageously also be used in the manufacture of tobacco products. Examples of such tobacco products include, cigars, cigarettes, pipe tobacco, chewing tobacco, and snuff. The manufacture of tobacco products supplemented with cooling additives is known per se.

In principle, the active substance content, i.e. the content of the coolant or coolant mixture according to the invention can vary over a wide range, such as 0.05 ppm to 10% by weight, preferably 0.1 ppm to 10% by weight.

The active ingredients according to the invention are also advantageously suitable for the production of packaging materials.

Production is also carried out in a manner known per se. The active ingredients can be incorporated into the packaging material in free or e.g. encapsulated form or applied to the packaging material in free or encapsulated form. In this way, appropriately equipped plastic packaging materials can be produced in accordance with the information in the literature on the production of polymer films. The production of suitably coated papers is also known to the skilled person.

Finally, the present invention relates to a method for modulating, in particular for in vitro and/or in vivo modulation, the cold menthol receptor TRPM8, comprising the following steps:
(i) providing at least one physiological coolant according to the invention or a physiological coolant mixture according to the invention or providing a cosmetic or pharmaceutical preparation according to the invention; and
(ii) contacting the coolant or coolant mixture or cosmetic or pharmaceutical preparation of step (i) with the receptor; or
   for producing a physiological cooling effect on the skin or mucous membrane, comprising the following steps:
(iii) providing at least one physiological coolant according to the invention or a physiological coolant mixture according to the invention or providing a cosmetic or pharmaceutical preparation according to the invention; and
(iv) the coolant or coolant mixture or cosmetic or pharmaceutical preparation of step (iii) with human skin or mucous membrane; or for improving the taste properties of flavoring substances comprising the following steps:
(v) providing at least one physiological coolant according to the invention or a physiological coolant mixture according to the invention or providing a cosmetic or pharmaceutical preparation according to the invention and at least one flavoring agent;
(vi) mixing of the two components; and, where appropriate
(vii) incorporation of the mixture into an oral preparation.

Further aspects of the present invention are apparent from the following examples and the appended patent claims.

### EXAMPLES

The following examples serve to illustrate the invention without limiting it. Unless otherwise stated, all data refer to the weight.

Preparation of active ingredients: The active ingredients/coolants used according to the invention can be prepared by a person skilled in the art in the field of organic synthesis using known synthesis methods, as described in more detail below.

### Cloning of Human TRPM8

The starting point for cloning the human TRPM8 receptor is an LnCaP cDNA bank. This is commercially available (e.g. BioChain, Hayward, USA) or can be produced from the andro gene-sensitive human prostate adenocarcinoma cell line LnCaP (e.g. ATCC, CRL1740 or ECACC, 891 1021 1) using standard kits.

The coding TRPM8 sequence (see e.g. http://www. ncbi.nlm.nih.gov/entrez/viewer.fcgi?db=nuccore&id=109689694) can be PCR-amplified and cloned using standard methods. The human TRPM8 gene isolated in this way was used to produce the plasmid plnd_M8. Alternatively, the TRPM8 gene can also be produced synthetically.

### Generation of HEK293 Test Cells

A stably transfected HEK293 cell line was produced as a test cell system using human TRPM8 DNA. Preference was given to HEK293, which offers the possibility of inducing TRPM8 expression using tetracycline via the plasmid introduced.

Methods for producing suitable test cell systems are known to the specialist and can be found in the relevant specialist literature.

### Assay on TRPM8 Modulators

A test similar to the test already described in the literature by Behrendt H. J. et al, Br. J. Pharmacol. 141, 2004, 737-745, is performed. The agonization or antagonization of the receptor can be quantified using a Ca²⁺-sensitive dye (e.g. FURA, Fluo-4 etc.). Agonists alone cause an increase in the Ca²⁺ signal; antagonists cause a reduction in the Ca²⁺ signal in the presence of e.g. menthol (detected in each case using the Fluo-4 dye, which has different fluorescence properties due to Ca²⁺ ions).

First, a fresh culture of transformed HEK cells is prepared in cell culture flasks in the usual way. The HEK293-TRPM8 test cells are detached from the cell culture flasks using trypsin and 40,000 cells/well are seeded with 100 μl medium in 96-well plates (Greiner #655948 poly-D-lysine-coated). To induce the TRPM8 receptor, tetracycline is added to the growth medium (DMEM/HG, 10% FCS tetracycline-free, 4 mM L-glutamine, 15 μg/ml blasticidin, 100 μg/ml hygromycin B, 1 μg/ml tetracycline).

On the following day, the cells are loaded with Fluo-4AM dye and the test is performed. Proceed as follows: Addition of 100 μl/well staining solution Ca-4 kit (RB 141, Molecular Devices) to each 100 μl medium (DMEM/HG, 10% FCS tetracycline-free, 4 mM L-glutamine, 15 μg/ml blasticidin, 100 μg/ml hygromycin B, 1 μg/ml tetracycline).

Incubation in incubator, 30 minutes/37° C./5% CO2, 30 minutes/RT.

Preparation of the test substances (different concentrations in 200 μl HBSS buffer), as well as positive controls (different concentrations of menthol, icilin or lonomycin in 200 μl HBSS buffer) and negative controls (only 200 μl HBSS buffer) Addition of the test substances in quantities of 50 μl/well and measurement of the fluorescence change (e.g. in the assay device FLIPR, Molecular Devices or NovoStar, BMG) at 485 nm excitation, 520 nm emission, and evaluation of the potency of the different substances/concentrations and determination of the ECG. e.g. in the FLIPR assay device, Molecular Devices or NovoStar, BMG) at 485 nm excitation, 520 nm emission, and evaluation of the potency of the various substances/concentrations and determination of the EC50 values.

The test substances are used in triplicates in concentrations of 0.1-200 μM in the assay. Normally, the compounds are kept ready in DMSO solutions and diluted down to a maximum DMSO concentration of 2% for the assay. Surprisingly, our own evaluations when performing the described assay showed that the compounds to be used according to the invention (as described herein) are particularly suitable as agonists of TRPM8.

The activity of the active substances in relation to activation of the TRPM8 channel is determined using the assay described. This is done in a concentration-dependent manner. For each active substance, 6 to 10 concentrations are measured as standard. A mathematical method (4-parameter or 5-parameter logistic curve fitting) can be used to determine the EC50 value as the inflection point of the sigmoidal curve from the activity values determined. These are standard biochemical methods that are familiar to experts.

The EC50 values determined for exemplary selected modulators according to the invention are shown in the following Table 7 and Table 8. An EC50 value of 1.72 μM was determined for the substance WS-3, which serves as a reference.

TABLE 7
EC50 values of modulators according to the invention

| | TRPM8 | | |
|---|---|---|---|
| | EC50 | | |
| Compound | [μM] | Structure | IUPAC name |
| B-01 | 0.05 | 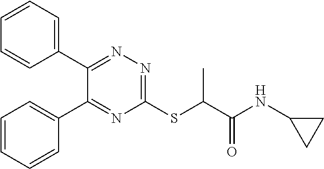 [SMILES: CC(Sc1nnc(-c2ccccc2)c(-c2ccccc2)n1)C(=O)NC1CC1] | N-Cyclopropyl-2-[(5,6- diphenyl-1,2,4-triazin-3- yl)sulfanyl]propanamide |
| B-02 | 0.08 | 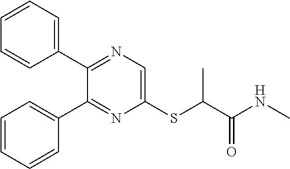 [SMILES: CNC(=O)C(C)Sc1cnc(-c2ccccc2)c(-c2ccccc2)n1] | 2-(5,6-diphenylpyrazin-2- yl)sulfanyl-N-methyl- propanamide |
| B-03 | 0.07 | 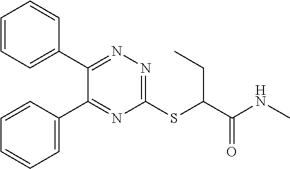 [SMILES: CCC(Sc1nnc(-c2ccccc2)c(-c2ccccc2)n1)C(=O)NC] | 2-[(5,6-diphenyl-1,2,4- triazin-3-yl)sulfanyl]-N- methyl-butanamide |
| B-04 | 0.37 | 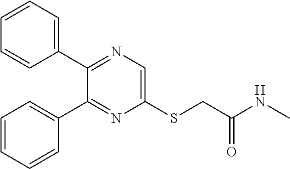 [SMILES: CNC(=O)CSc1cnc(-c2ccccc2)c(-c2ccccc2)n1] | 2-(5,6-diphenylpyrazin-2- yl)sulfanyl-N-methyl- acetamide |
| B-05 | 0.1 | 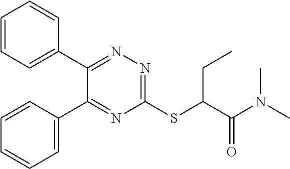 [SMILES: CCC(Sc1nnc(-c2ccccc2)c(-c2ccccc2)n1)C(=O)N(C)C] | 2-[(5,6-diphenyl-1,2,4- triazin-3-yl)sulfanyl]-N,N- dimethyl-butanamide |
| B-06 | 0.28 | 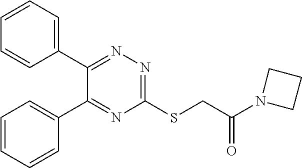 [SMILES: O=C(CSc1nnc(-c2ccccc2)c(-c2ccccc2)n1)N1CCC1] | 1-(azetidin-1-yl)-2-[(5,6- diphenyl-1,2,4-triazin-3- yl)sulfanyl]ethanone |
| B-07 | 0.92 | 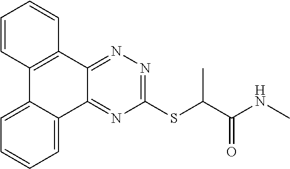 [SMILES: CNC(=O)C(C)Sc1nnc2c3ccccc3c3ccccc3c2n1] | N-methyl-2- phenanthro[9,10- e][1,2,4]triazine-3- ylsulfanyl-propanamide |
| B-11 | 1.43 | 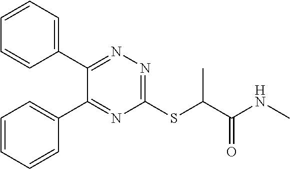 [SMILES: CNC(=O)C(C)Sc1nnc(-c2ccccc2)c(-c2ccccc2)n1] | 2-[(5,6-diphenyl-1,2,4- triazin-3-yl)sulfanyl]-N- methyl-propanamide |
| B-14 | | 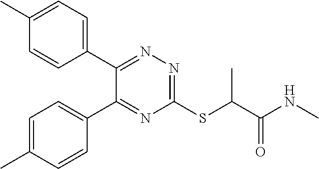 | 2-[[5,6-bis(p-tolyl)-1,2,4- triazin-3-yl]sulfanyl]-N- methyl-propanamide |
| B-15 | | 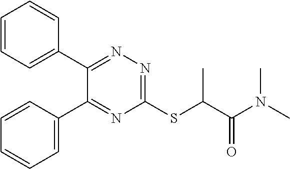 [SMILES: CC(Sc1nnc(-c2ccccc2)c(-c2ccccc2)n1)C(=O)N(C)C] | 2-[(5,6-diphenyl-1,2,4- triazin-3-yl)sulfanyl]-N,N- dimethyl-propanamide |
| B-17 | | 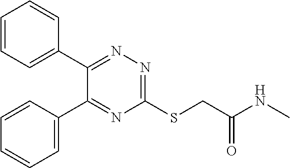 [SMILES: CNC(=O)CSc1nnc(-c2ccccc2)c(-c2ccccc2)n1] | 2-[(5,6-diphenyl-1,2,4- triazin-3-yl)sulfanyl-N- methyl-acetamide |
| B-18 | 5.36 | 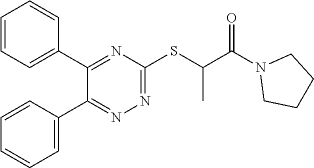 [SMILES: CC(Sc1nnc(-c2ccccc2)c(-c2ccccc2)n1)C(=O)N1CCCC1] | 2-[(5,6-diphenyl-1,2,4- triazin-3-yl)sulfanyl]-1- pyrrolidin-1-yl-propan-1- one |
| B-19 | | 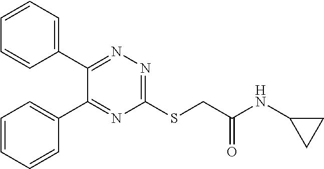 [SMILES: O=C(CSc1nnc(-c2ccccc2)c(-c2ccccc2)n1)NC1CC1] | N-cyclopropyl-2-[(5,6- diphenyl-1,2,4-triazin-3- yl)sulfanyl]acetamide |
| B-21 | | | 2-[[5,6-bis(p-tolyl)-1,2,4- |
| | | | triazin-3-yl]sulfanyl]-N-(2- |
| | | | methoxyethyl)butanamide |
| B-22 | | 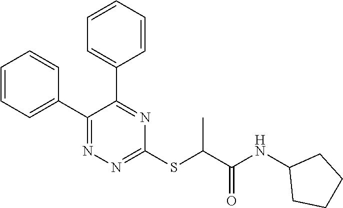 [SMILES: CC(Sc1nnc(-c2ccccc2)c(-c2ccccc2)n1)C(=O)NC1CCCC1] | N-cyclopentyl-2-[(5,6- diphenyl-1,2,4-triazin-3- yl)sulfanyl]propanamide |
| B-23 | | 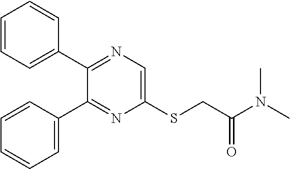 [SMILES: CN(C)C(=O)CSc1cnc(-c2ccccc2)c(-c2ccccc2)n1] | 2-(5,6-diphenylpyrazin-2- yl)sulfanyl-N,N-dimethyl- acetamide |
| B-24 | | 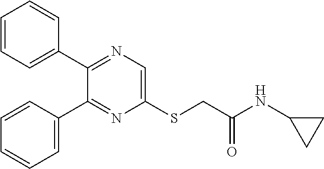 [SMILES: O=C(CSc1cnc(-c2ccccc2)c(-c2ccccc2)n1)NC1CC1] | N-cyclopropyl-2-(5,6- diphenylpyrazin-2- yl)sulfanyl-acetamide |
| B-25 | | 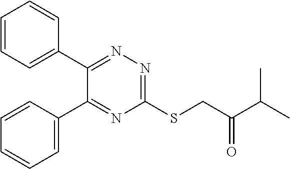 [SMILES: CC(C)C(=O)CSc1nnc(-c2ccccc2)c(-c2ccccc2)n1] | 1-[(5,6-diphenyl-1,2,4- triazin-3-yl)sulfanyl]-3- methyl-butan-2-one |
| B-26 | | 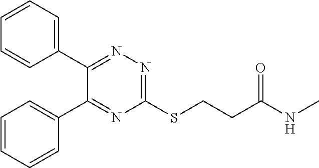 [SMILES: CNC(=O)CCSc1nnc(-c2ccccc2)c(-c2ccccc2)n1] | 3-[(5,6-diphenyl-1,2,4- triazin-3-yl)sulfanyl]-N- methyl-propanamide |
| B-27 | | 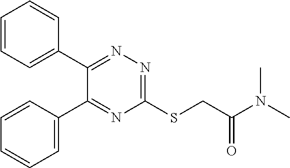 [SMILES: CN(C)C(=O)CSc1nnc(-c2ccccc2)c(-c2ccccc2)n1] | 2-[(5,6-diphenyl-1,2,4- triazin-3-yl)sulfanyl]-N,N- dimethyl-acetamide |
| B-28 | | 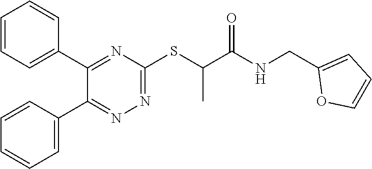 [SMILES: CC(Sc1nnc(-c2ccccc2)c(-c2ccccc2)n1)C(=O)NCc1ccco1] | 2-[(5,6-diphenyl-1,2,4- triazin-3-yl)sulfanyl]-N-(2- furylmethyl)propanamide |
| B-29 | | 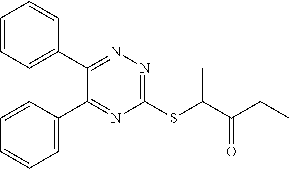 [SMILES: CCC(=O)C(C)Sc1nnc(-c2ccccc2)c(-c2ccccc2)n1] | 2-[(5,6-diphenyl-1,2,4- triazin-3- yl)sulfanyl]pentan-3-one |
| B-30 | | 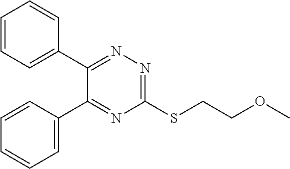 [SMILES: COCCSc1nnc(-c2ccccc2)c(-c2ccccc2)n1] | 3-(2- methoxyethylsulfanyl)- 5,6-diphenyl-1,2,4- triazine |
| B-31 | | 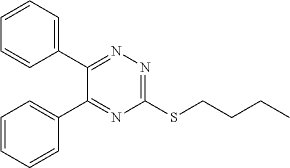 [SMILES: CCCCSc1nnc(-c2ccccc2)c(-c2ccccc2)n1] | 3-butylsulfanyl-5,6- diphenyl-1,2,4-triazine |
| B-32 | | 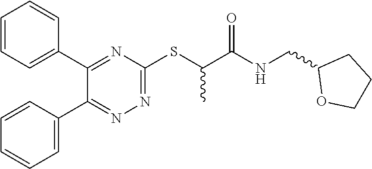 [SMILES: CC(Sc1nnc(-c2ccccc2)c(-c2ccccc2)n1)C(=O)NCC1CCCO1] | 2-[(5,6-diphenyl-1,2,4- triazin-3-yl)sulfanyl]-N- (tetrahydrofuran-2- ylmethyl)propanamide |
| B-33 | | 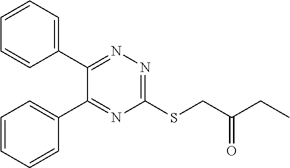 [SMILES: CCC(=O)CSc1nnc(-c2ccccc2)c(-c2ccccc2)n1] | 1-[(5,6-diphenyl-1,2,4- triazin-3- yl)sulfanyl]butan-2-one |
| B34 | | 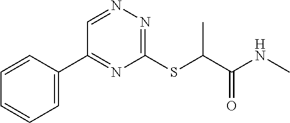 [SMILES: CNC(=O)C(C)Sc1nncc(-c2ccccc2)n1] | N-methyl-2-[(5-phenyl- 1,2,4-triazin-3- yl)sulfanyl]propanamide |
| B36 | | 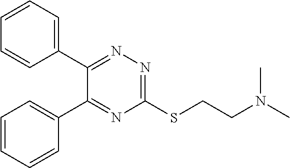 [SMILES: CN(C)CCSc1nnc(-c2ccccc2)c(-c2ccccc2)n1] | 2-[(5,6-diphenyl-1,2,4- triazin-3-yl)sulfanyl]-N,N- dimethyl-ethanamide |
| B-37 | | 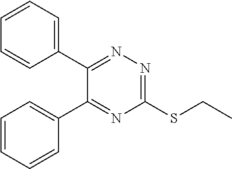 [SMILES: CCSc1nnc(-c2ccccc2)c(-c2ccccc2)n1] | 3-ethylsulfanyl-5,6- diphenyl-1,2,4-triazine |
| B-38 | | 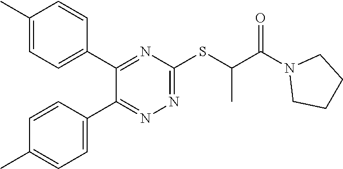 [SMILES: Cc1ccc(-c2nnc(SC(C)C(=O)N3CCCC3)nc2-c2ccc(C)cc2)cc1] | 2-[[5,6-bis(p-tolyl)-1,2,4- triazin-3-yl]sulfanyl]-1- pyrrolidin-1-yl-propan-1- one |
| B-39 | | 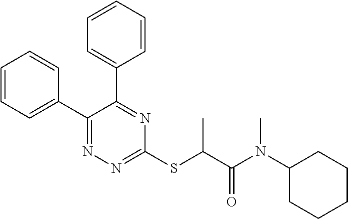 [SMILES: CC(Sc1nnc(-c2ccccc2)c(-c2ccccc2)n1)C(=O)N(C)C1CCCCC1] | N-cyclohexyl-2-[(5,6- diphenyl-1,2,4-triazin-3- yl)sulfanyl]-N-methyl- propanamide |
| B-40 | | 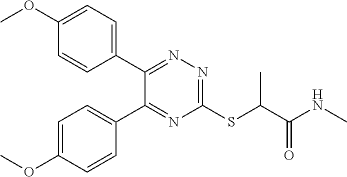 | 2-[[5,6-bis(4- methoxyphenyl)-1,2,4- triazin-3-yl]sulfanyl]-N- methyl-propanamide |
| B-41 | | 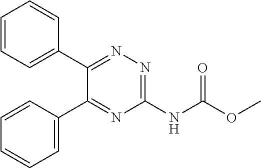 [SMILES: COC(=O)Nc1nnc(-c2ccccc2)c(-c2ccccc2)n1] | methyl N-(5,6-diphenyl- 1,2,4-triazin-3- yl)carbamate |
| B-42 | | 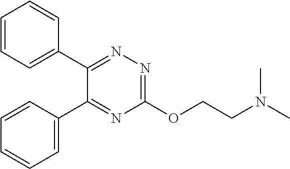 [SMILES: CN(C)CCOc1nnc(-c2ccccc2)c(-c2ccccc2)n1] | 2-[(5,6-diphenyl-1,2,4- triazin-3-yl)oxy]-N,N- dimethyl-ethanamine |
| B-43 | | 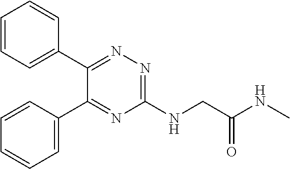 [SMILES: CNC(=O)CNc1nnc(-c2ccccc2)c(-c2ccccc2)n1] | 2-[(5,6-diphenyl-1,2,4- triazin-3-yl)amino]-N- methyl-acetamide |
| B-44 | | 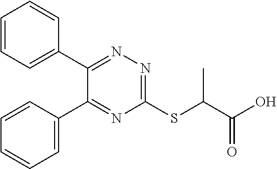 [SMILES: CC(Sc1nnc(-c2ccccc2)c(-c2ccccc2)n1)C(=O)O] | 2-[(5,6-diphenyl-1,2,4- triazin-3- yl)sulfanyl]propanoic acid |
| B-45 | | 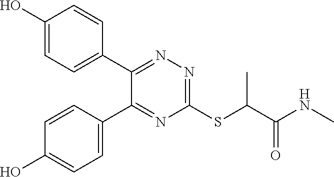 [SMILES: CNC(=O)C(C)Sc1nnc(-c2ccc(O)cc2)c(-c2ccc(O)cc2)n1] | 2-[[5,6-bis(4- hydroxyphenyl)-1,2,4- triazin-3-yl]sulfanyl]-N- methyl-propanamide |
| B-46 | | 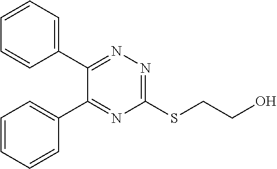 [SMILES: OCCSc1nnc(-c2ccccc2)c(-c2ccccc2)n1] | 2-[(5,6-diphenyl-1,2,4- triazin-3- yl)sulfanyl]ethanol |
| B-47 | | 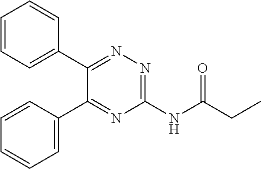 [SMILES: CCC(=O)Nc1nnc(-c2ccccc2)c(-c2ccccc2)n1] | N-(5,6-diphenyl-1,2,4- triazin-3-yl)propanamide |
| B-48 | | 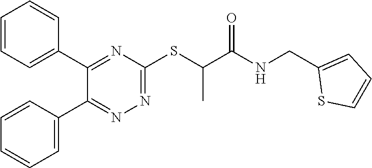 [SMILES: CC(Sc1nnc(-c2ccccc2)c(-c2ccccc2)n1)C(=O)NCc1cccs1] | 2-[(5,6-diphenyl-1,2,4- triazin-3-yl)sulfanyl]-N-(2- thienylmethyl)propanamide |
| B-49 | | 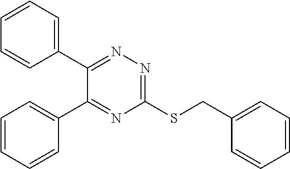 [SMILES: c1ccc(CSc2nnc(-c3ccccc3)c(-c3ccccc3)n2)cc1] | 3-benzylsulfanyl-5,6- diphenyl-1,2,4-triazine |
| B-50 | | 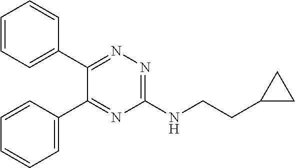 [SMILES: c1ccc(-c2nnc(NCCC3CC3)nc2-c2ccccc2)cc1] | N-(2-cyclopropylethyl)- 5,6-diphenyl-1,2,4- triazin-3-amine |
| B-51 | | 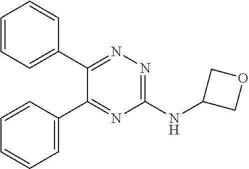 [SMILES: c1ccc(-c2nnc(NC3COC3)nc2-c2ccccc2)cc1] | N-(oxetan-3-yl)-5,6- diphenyl-1,2,4-triazin-3- amine |
| B-52 | | 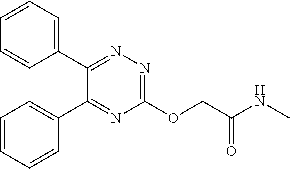 [SMILES: CNC(=O)COc1nnc(-c2ccccc2)c(-c2ccccc2)n1] | 2-[(5,6-diphenyl-1,2,4- triazin-3-yl)oxy]-N- methyl-acetamide |
| B-53 | | 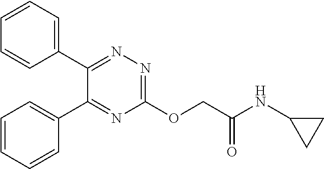 [SMILES: O=C(COc1nnc(-c2ccccc2)c(-c2ccccc2)n1)NC1CC1] | N-cyclopropyl-2-[(5,6- diphenyl-1,2,4-triazin-3- yl)oxy]acetamide |
| B-54 | | 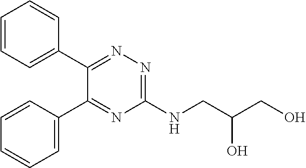 [SMILES: OCC(O)CNc1nnc(-c2ccccc2)c(-c2ccccc2)n1] | 3-[(5,6-diphenyl-1,2,4- triazin-3- yl)amino]propane-1,2- diol |
| B-55 | | 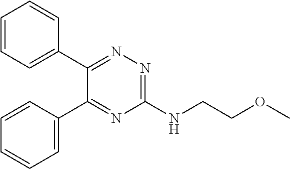 [SMILES: COCCNc1nnc(-c2ccccc2)c(-c2ccccc2)n1] | N-(2-methoxyethyl)-5,6- diphenyl-1,2,4-triazin-3- amine |
| B-56 | | 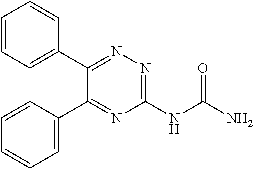 [SMILES: NC(=O)Nc1nnc(-c2ccccc2)c(-c2ccccc2)n1] | (5,6-diphenyl-1,2,4- triazin-3-yl)urea |
| B-57 | | 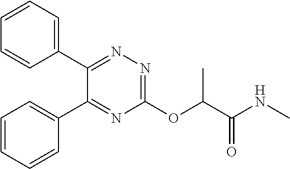 [SMILES: CNC(=O)C(C)Oc1nnc(-c2ccccc2)c(-c2ccccc2)n1] | 2-[(5,6-diphenyl-1,2,4- triazin-3-yl)oxy]-N- methyl-propanamide |
| B-58 | | 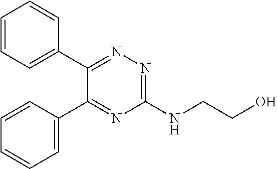 [SMILES: OCCNc1nnc(-c2ccccc2)c(-c2ccccc2)n1] | 2-[(5,6-diphenyl-1,2,4- triazin-3-yl)amino]ethanol |
| B-59 | | 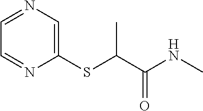 | N-methyl-2-pyrazine-2- ylsulfanyl-propanamide |
| B-60 | | 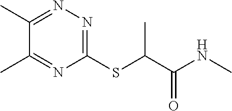 | 2-[(5,6-dimethyl-1,2,4- triazin-3-yl)sulfanyl]-N- methyl-propanamide |
| B-61 | | 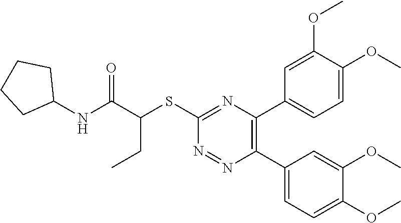 [SMILES: CCC(Sc1nnc(-c2ccc(OC)c(OC)c2)c(-c2ccc(OC)c(OC)c2)n1)C(=O)NC1CCCC1] | 2-[[5,6-bis(3,4- dimethoxyphenyl)-1,2,4- triazin-3-yl]sulfanyl]-N- cyclopentyl-butanamide |
| B-62 | | 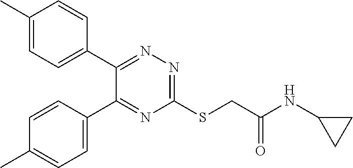 [SMILES: Cc1ccc(-c2nnc(SCC(=O)NC3CC3)nc2-c2ccc(C)cc2)cc1] | 2-[[5,6-bis(p-tolyl)-1,2,4- triazin-3-yl]sulfanyl]-N- cyclopropyl-acetamide |
| B-63 | | 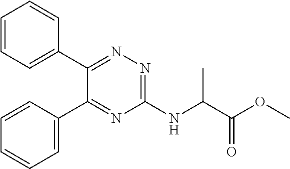 [SMILES: COC(=O)C(C)Nc1nnc(-c2ccccc2)c(-c2ccccc2)n1] | methyl 2-[(5,6-diphenyl- 1,2,4-triazin-3- yl)amino]propanoate |
| B-64 | | 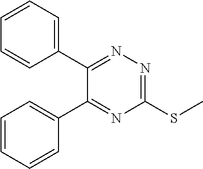 [SMILES: CSc1nnc(-c2ccccc2)c(-c2ccccc2)n1] | 3-methylsulfanyl-5,6- diphenyl-1,2,4-triazine |
| B-65 | | 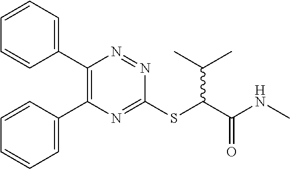 [SMILES: CNC(=O)C(Sc1nnc(-c2ccccc2)c(-c2ccccc2)n1)C(C)C] | 2-[(5,6-diphenyl-1,2,4- triazin-3-yl)sulfanyl]-N,3- dimethyl-butanamide |
| B-66 | | 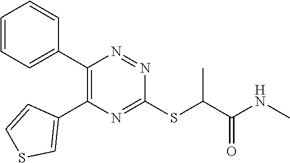 [SMILES: CNC(=O)C(C)Sc1nnc(-c2ccccc2)c(-c2ccsc2)n1] | N-methyl-2-[[6-phenyl-5- (3-thienyl)-1,2,4-triazin-3- yl]sulfanyl]propanamide |
| B-67 | | 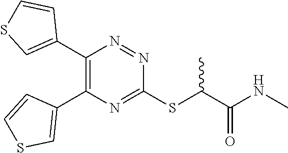 [SMILES: CNC(=O)C(C)Sc1nnc(-c2ccsc2)c(-c2ccsc2)n1] | 2-[[5,6-bis(3-thienyl)- 1,2,4-triazin-3- yl]sulfanyl]-N-methyl- propanamide |
| B-68 | | 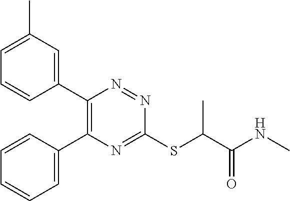 [SMILES: CNC(=O)C(C)Sc1nnc(-c2cccc(C)c2)c(-c2ccccc2)n1] | N-methyl-2-[[6-(m-tolyl)- 5-phenyl-1,2,4-triazin-3- yl]sulfanyl]propanamide |
| B-69 | | 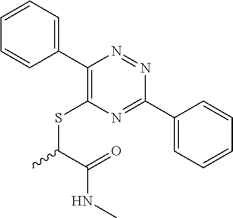 [SMILES: CNC(=O)C(C)Sc1nc(-c2ccccc2)nnc1-c1ccccc1] | 2-[(3,6-diphenyl-1,2,4- triazin-5-yl)sulfanyl]-N- methyl-propanamide |
| B-70 | | 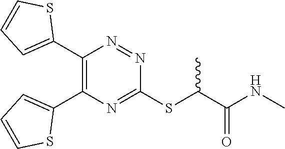 [SMILES: CNC(=O)C(C)Sc1nnc(-c2cccs2)c(-c2cccs2)n1] | 2-[[5,6-bis(2-thienyl)- 1,2,4-triazin-3- yl]sulfanyl]-N-methyl- propanamide |
| B-71 | | 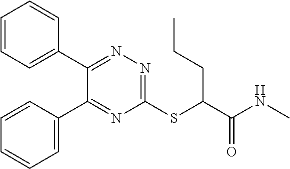 [SMILES: CCCC(Sc1nnc(-c2ccccc2)c(-c2ccccc2)n1)C(=O)NC] | 2-[(5,6-diphenyl-1,2,4- triazin-3-yl)sulfanyl]-N- methyl-pentanamide |
| B-72 | | 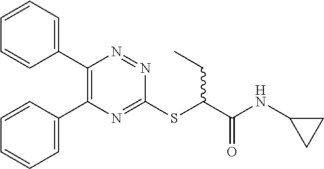 [SMILES: CCC(Sc1nnc(-c2ccccc2)c(-c2ccccc2)n1)C(=O)NC1CC1] | N-cyclopropyl-2-[(5,6- diphenyl-1,2,4-triazin-3- yl)sulfanyl]butanamide |
| B-73 | | 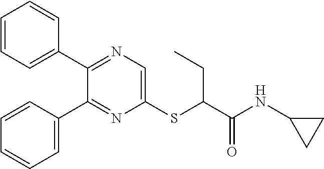 [SMILES: CCC(Sc1cnc(-c2ccccc2)c(-c2ccccc2)n1)C(=O)NC1CC1] | N-cyclopropyl-2-(5,6- diphenylpyrazin-2- yl)sulfanyl-butanamide |
| B-74 | | 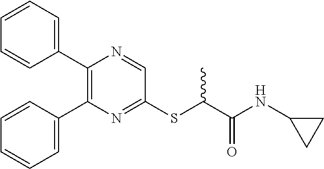 [SMILES: CC(Sc1cnc(-c2ccccc2)c(-c2ccccc2)n1)C(=O)NC1CC1] | N-cyclopropyl-2-(5,6- diphenylpyrazin-2- yl)sulfanyl-propanamide |
| B-75 | | 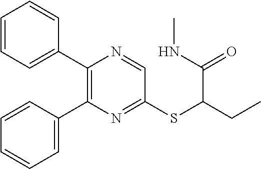 [SMILES: CCC(Sc1cnc(-c2ccccc2)c(-c2ccccc2)n1)C(=O)NC] | 2-[(5,6-Diphenylpyrazin- 2-yl)sulfanyl]-N- methylbutanamide |
| B-76 | | 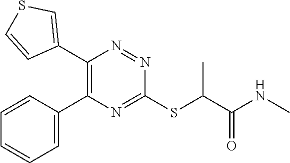 [SMILES: CNC(=O)C(C)Sc1nnc(-c2ccsc2)c(-c2ccccc2)n1] | N-methyl-2-[[5-phenyl-6- (3-thienyl)-1,2,4-triazin-3- yl]sulfanyl]propanamide |
| B-77 | | 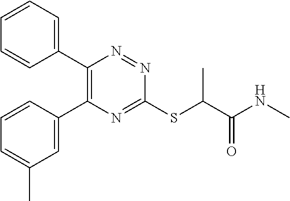 [SMILES: CNC(=O)C(C)Sc1nnc(-c2ccccc2)c(-c2cccc(C)c2)n1] | N-methyl-2-[[5-(m-tolyl)- 6-phenyl-1,2,4-triazin-3- yl]sulfanyl]propanamide |

TABLE 8
EC50 values of modulators according to the invention

| | TRPM8 | | |
|---|---|---|---|
| | EC50 | | |
| Compound | [μM] | Structure | IUPAC name |
| A-01 | 0.01 | 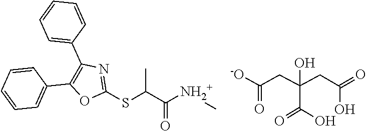 | 3-carboxy-3,5-dihydroxy- 5-oxo-pentanoate; 2-(4,5- diphenyloxazol-2-yl)sul- fanylpropanoyl-methyl- ammonium |
| A-02 | 0.01 | 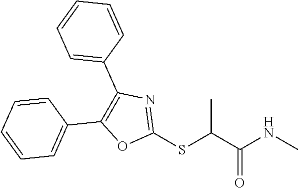 [SMILES: CNC(=O)C(C)Sc1nc(-c2ccccc2)c(-c2ccccc2)o1] | 2-(4,5-diphenyloxazol-2- yl)sulfanyl-N-methyl- propanamide |
| A-03 | 0.01 | 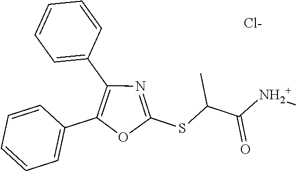 [SMILES: C[NH2+]C(=O)C(C)Sc1nc(-c2ccccc2)c(-c2ccccc2)o1 ; [Cl-]] | 2-(4,5-diphenyloxazol- 2-yl)sulfanylpropanoyl- methyl-ammonium; chloride |
| A-04 | 0.6 | 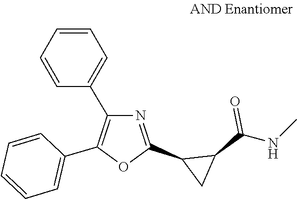 [SMILES: CNC(=O)[C@H]1C[C@H]1c1nc(-c2ccccc2)c(-c2ccccc2)o1] | rac-(1S,2R)-2-(4,5- diphenyloxazol-2-yl)-N- methyl-cyclopropane- carboxamides |
| A-05 | 2.31 | 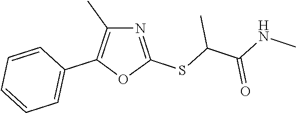 [SMILES: CNC(=O)C(C)Sc1nc(C)c(-c2ccccc2)o1] | N-methyl-2-(4-methyl-5- phenyl-oxazol-2-yl)sul- fanyl-propanamide |
| A-06 | 0.34 | 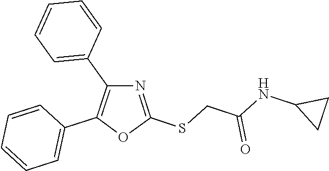 [SMILES: O=C(CSc1nc(-c2ccccc2)c(-c2ccccc2)o1)NC1CC1] | N-cyclopropyl-2-(4,5- diphenyloxazol-2-yl)sul- fanyl-acetamide |
| A-07 | 0.15 | 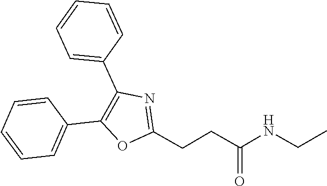 [SMILES: CCNC(=O)CCc1nc(-c2ccccc2)c(-c2ccccc2)o1] | 3-(4,5-diphenyloxazol-2- yl)-N-ethyl-propanamide |
| A-08 | 0.11 | 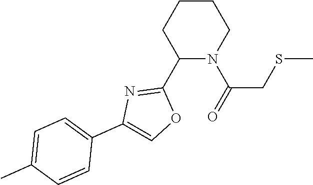 | 2-methylsulfanyl-1-[2-[4- (p-tolyl)oxazol-2-yl]-1- piperidyl]ethanone |
| A-09 | 1.54 | 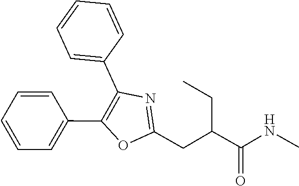 [SMILES: CCC(Cc1nc(-c2ccccc2)c(-c2ccccc2)o1)C(=O)NC] | 2-(4,5-diphenyloxazol-2- yl)sulfanyl-N-methyl- butanamide |
| A-10 | 2.13 | 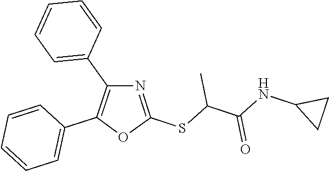 [SMILES: CC(Sc1nc(-c2ccccc2)c(-c2ccccc2)o1)C(=O)NC1CC1] | N-cyclopropyl-2-(4,5- diphenyloxazol-2-yl)sul- fanyl-propanamide |
| A-11 | | 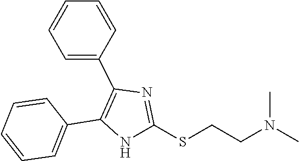 [SMILES: CN(C)CCSc1nc(-c2ccccc2)c(-c2ccccc2)[nH]1] | 2-[(4,5-diphenyl-1H- imidazol-2-yl)sulfanyl]- N,N-dimethyl-ethanamine |
| A-12 | 5.09 | 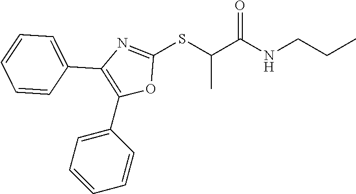 [SMILES: CCCNC(=O)C(C)Sc1nc(-c2ccccc2)c(-c2ccccc2)o1] | 2-(4,5-diphenyloxazol-2- yl)sulfanyl-N-propyl- propanamide |
| A-15 | 7.14 | 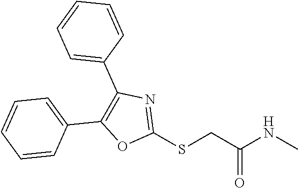 [SMILES: CNC(=O)CSc1nc(-c2ccccc2)c(-c2ccccc2)o1] | 2-(4,5-diphenyloxazol-2- yl)sulfanyl-N-methyl- acetamide |
| A-16 | 4.27 | 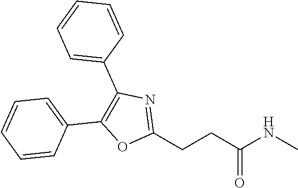 [SMILES: CNC(=O)CCc1nc(-c2ccccc2)c(-c2ccccc2)o1] | 3-(4,5-diphenyloxazol-2- yl)-N-methyl-propanamide |
| A-17 | 5.85 | 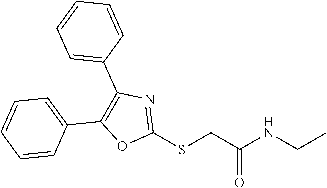 [SMILES: CCNC(=O)CSc1nc(-c2ccccc2)c(-c2ccccc2)o1] | 2-(4,5-diphenyloxazol-2- yl)sulfanyl-N-ethyl-acet- amide |
| A-18 | | 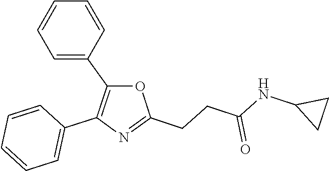 [SMILES: O=C(CCc1nc(-c2ccccc2)c(-c2ccccc2)o1)NC1CC1] | N-cyclopropyl-3-(4,5- diphenyloxazol-2-yl)- propanamide |
| A-19 | | 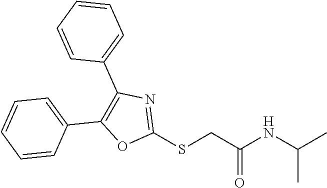 [SMILES: CC(C)NC(=O)CSc1nc(-c2ccccc2)c(-c2ccccc2)o1] | 2-(4,5-diphenyloxazol-2- yl)sulfanyl-N-isopropyl- acetamide |
| A-20 | | 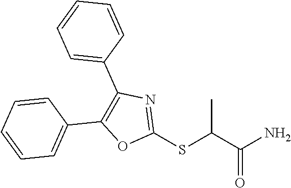 [SMILES: CC(Sc1nc(-c2ccccc2)c(-c2ccccc2)o1)C(N)=O] | 2-(4,5-diphenyloxazol-2- yl)sulfanylpropanamide |
| A-21 | | 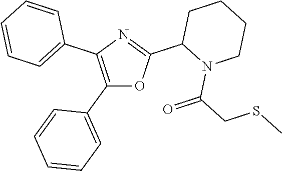 [SMILES: CSCC(=O)N1CCCCC1c1nc(-c2ccccc2)c(-c2ccccc2)o1] | 1-[2-(4,5-diphenyloxazol- 2-yl)-1-piperidyl]-2-meth- ylsulfanyl-ethanone |
| A-22 | | 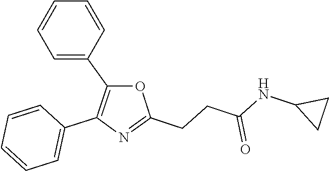 [SMILES: O=C(CCc1nc(-c2ccccc2)c(-c2ccccc2)o1)NC1CC1] | N-cyclopropyl-3-(4,5- diphenyloxazol-2-yl)- propanamide |
| A-23 | | 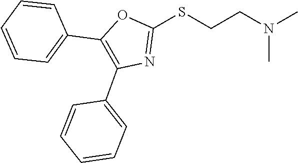 [SMILES: CN(C)CCSc1nc(-c2ccccc2)c(-c2ccccc2)o1] | 2-(4,5-diphenyloxazol-2- yl)sulfanyl-N,N-dimethyl- ethanamine |
| A-24 | 11.27 | 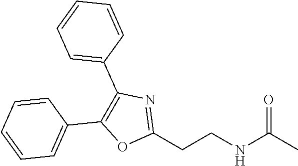 [SMILES: CC(=O)NCCc1nc(-c2ccccc2)c(-c2ccccc2)o1] | N-[2-(4,5-diphenyloxazol- 2-yl)ethyl]acetamide |
| A-25 | | 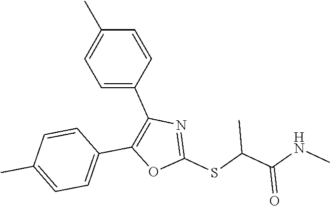 | 2-[4,5-bis(p-tolyl)oxazol- 2-yl]sulfanyl-N-methyl- propanamide |
| A-26 | | 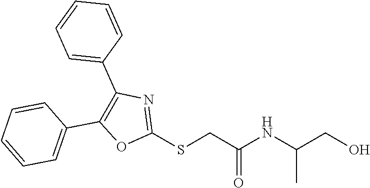 [SMILES: CC(CO)NC(=O)CSc1nc(-c2ccccc2)c(-c2ccccc2)o1] | 2-(4,5-diphenyloxazol-2- yl)sulfanyl-N-(2-hydroxy- 1-methyl-ethyl)acetamide |
| A-27 | 18.04 | 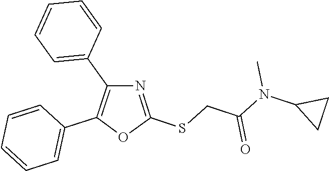 [SMILES: CN(C(=O)CSc1nc(-c2ccccc2)c(-c2ccccc2)o1)C1CC1] | N-cyclopropyl-2-(4,5- diphenyloxazol-2-yl)sul- fanyl-N-methyl-acetamide |
| A-28 | | 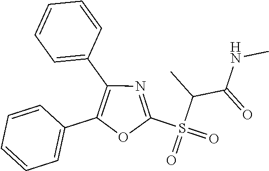 [SMILES: CNC(=O)C(C)S(=O)(=O)c1nc(-c2ccccc2)c(-c2ccccc2)o1] | 2-(4,5-diphenyloxazol-2- yl)sulfonyl-N-methyl- propanamide |
| A-29 | | 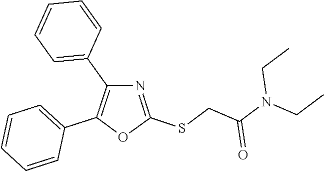 [SMILES: CCN(CC)C(=O)CSc1nc(-c2ccccc2)c(-c2ccccc2)o1] | 2-(4,5-diphenyloxazol-2- yl)sulfanyl-N,N-diethyl- acetamide |
| A-30 | | 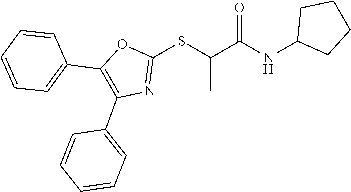 [SMILES: CC(Sc1nc(-c2ccccc2)c(-c2ccccc2)o1)C(=O)NC1CCCC1] | N-cyclopentyl-2-(4,5- diphenyloxazol-2-yl)sul- fanyl-propanamide |
| A-31 | | 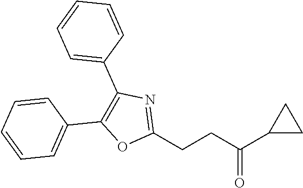 [SMILES: O=C(CCc1nc(-c2ccccc2)c(-c2ccccc2)o1)C1CC1] | 1-cyclopropyl-3-(4,5- diphenyloxazol-2-yl)- propan-1-one |
| A-32 | | 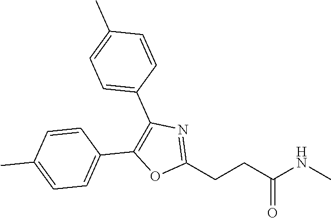 | 3-[4,5-bis(p-tolyl)oxazol- 2-yl]-N-methyl-propan- amide |
| A-33 | | 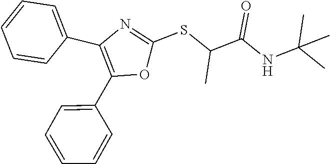 [SMILES: CC(Sc1nc(-c2ccccc2)c(-c2ccccc2)o1)C(=O)NC(C)(C)C] | N-tert-butyl-2-(4,5- diphenyloxazol-2-yl)sul- fanyl-propanamide |
| A-34 | | 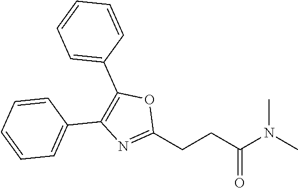 [SMILES: CN(C)C(=O)CCc1nc(-c2ccccc2)c(-c2ccccc2)o1] | 3-(4,5-diphenyloxazol-2- yl)-N,N-dimethyl-propan- amide |
| A-35 | | 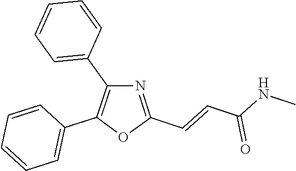 [SMILES: CNC(=O)/C=C/c1nc(-c2ccccc2)c(-c2ccccc2)o1] | (E)-3-(4,5-diphenyloxazol- 2-yl)-N-methyl-prop-2- enamide |
| A-36 | | 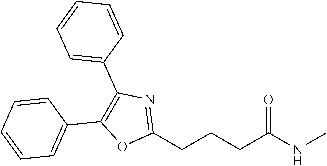 [SMILES: CNC(=O)CCCc1nc(-c2ccccc2)c(-c2ccccc2)o1] | 4-(4,5-diphenyloxazol-2- yl)-N-methyl-butanamide |
| A-37 | | 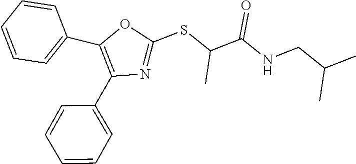 [SMILES: CC(C)CNC(=O)C(C)Sc1nc(-c2ccccc2)c(-c2ccccc2)o1] | 2-(4,5-diphenyloxazol-2- yl)sulfanyl-N-isobutyl- propanamide |
| A-38 | | 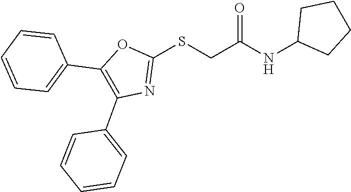 [SMILES: O=C(CSc1nc(-c2ccccc2)c(-c2ccccc2)o1)NC1CCCC1] | N-cyclopentyl-2-(4,5- diphenyloxazol-2-yl)sul- fanyl-acetamide |
| A-39 | | 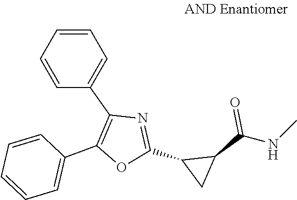 [SMILES: CNC(=O)[C@H]1C[C@@H]1c1nc(-c2ccccc2)c(-c2ccccc2)o1] | rac-(1S,2S)-2-(4,5- diphenyloxazol-2-yl)-N- methyl-cyclopropane- carboxamides |
| A-40 | | 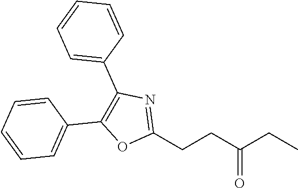 [SMILES: CCC(=O)CCc1nc(-c2ccccc2)c(-c2ccccc2)o1] | 1-(4,5-diphenyloxazol-2- yl)pentan-3-one |
| A-42 | | 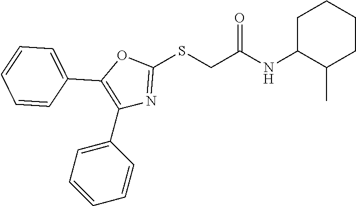 [SMILES: CC1CCCCC1NC(=O)CSc1nc(-c2ccccc2)c(-c2ccccc2)o1] | 2-(4,5-diphenyloxazol-2- yl)sulfanyl-N-(2-meth- ylcyclohexyl)acetamide |
| A-43 | | 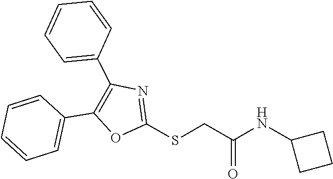 [SMILES: O=C(CSc1nc(-c2ccccc2)c(-c2ccccc2)o1)NC1CCC1] | N-cyclobutyl-2-(4,5- diphenyloxazol-2-yl)sul- fanyl-acetamide |
| A-44 | | 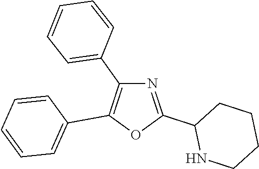 [SMILES: c1ccc(-c2nc(C3CCCCN3)oc2-c2ccccc2)cc1] | 4,5-diphenyl-2-(2-piperid- yl)oxazole |
| A-45 | | 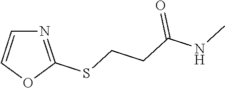 | N-methyl-3-oxazol-2-yl- sulfanyl-propanamide |
| A-46 | | 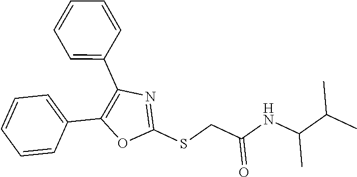 [SMILES: CC(C)C(C)NC(=O)CSc1nc(-c2ccccc2)c(-c2ccccc2)o1] | N-(1,2-dimethylpropyl)-2- (4,5-diphenyloxazol-2- yl)sulfanyl-acetamide |
| A-47 | | 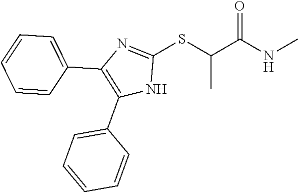 [SMILES: CNC(=O)C(C)Sc1nc(-c2ccccc2)c(-c2ccccc2)[nH]1] | 2-[(4,5-diphenyl-1H- imidazol-2-yl)sulfanyl]- N-methyl-propanamide |
| A-48 | | 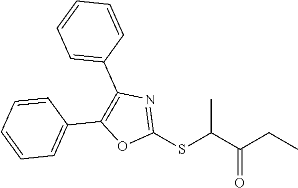 [SMILES: CCC(=O)C(C)Sc1nc(-c2ccccc2)c(-c2ccccc2)o1] | 2-(4,5-diphenyloxazol-2- yl)sulfanylpentan-3-one |
| A-49 | | 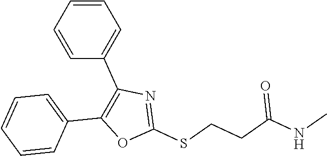 [SMILES: CNC(=O)CCSc1nc(-c2ccccc2)c(-c2ccccc2)o1] | 3-(4,5-diphenyloxazol-2- yl)sulfanyl-N-methyl- propanamide |
| A-50 | | 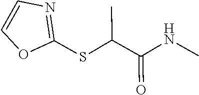 | N-methyl-2-oxazol-2-yl- sulfanyl-propanamide |
| A-51 | | 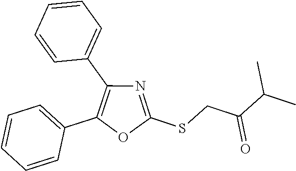 [SMILES: CC(C)C(=O)CSc1nc(-c2ccccc2)c(-c2ccccc2)o1] | 1-(4,5-diphenyloxazol- 2-yl)sulfanyl-3-methyl- butan-2-one |
| A-52 | | 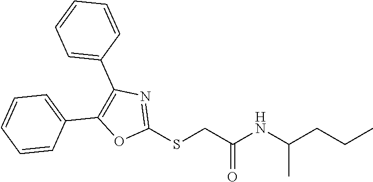 [SMILES: CCCC(C)NC(=O)CSc1nc(-c2ccccc2)c(-c2ccccc2)o1] | 2-(4,5-diphenyloxazol-2- yl)sulfanyl-N-(1-meth- ylbutyl)acetamide |
| A-53 | | 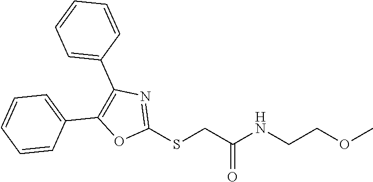 [SMILES: COCCNC(=O)CSc1nc(-c2ccccc2)c(-c2ccccc2)o1] | 2-(4,5-diphenyloxazol-2- yl)sulfanyl-N-(2-meth- oxyethyl)acetamide |
| A-54 | | 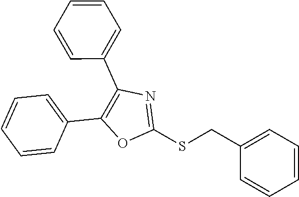 [SMILES: c1ccc(CSc2nc(-c3ccccc3)c(-c3ccccc3)o2)cc1] | 2-benzylsulfanyl-4,5- diphenyl-oxazole |
| A-55 | | 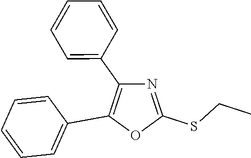 [SMILES: CCSc1nc(-c2ccccc2)c(-c2ccccc2)o1] | 2-ethylsulfanyl-4,5- diphenyl-oxazole |
| A-56 | | 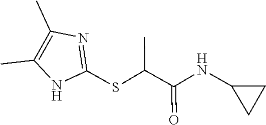 [SMILES: Cc1nc(SC(C)C(=O)NC2CC2)[nH]c1C] | N-cyclopropyl-2-[(4,5- dimethyl-1H-imidazol-2- yl)sulfanyl]propanamide |
| A-57 | | 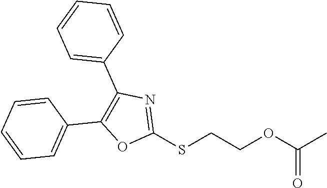 [SMILES: CC(=O)OCCSc1nc(-c2ccccc2)c(-c2ccccc2)o1] | 2-(4,5-diphenyloxazol-2- yl)sulfanylethyl acetate |
| A-58 | | 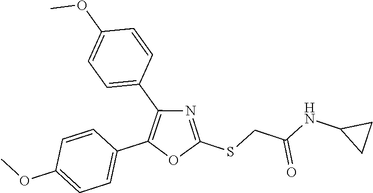 [SMILES: COc1ccc(-c2nc(SCC(=O)NC3CC3)oc2-c2ccc(OC)cc2)cc1] | 2-[4,5-bis(4-methoxy- phenyl)oxazol-2-yl]sul- fanyl-N-cyclopropyl- acetamide |
| A-59 | | 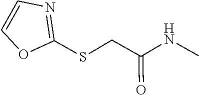 | N-methyl-2-oxazol-2- ylsulfanyl-acetamide |
| A-60 | | 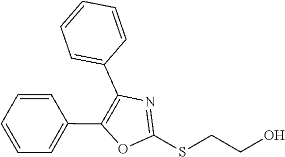 [SMILES: OCCSc1nc(-c2ccccc2)c(-c2ccccc2)o1] | 2-(4,5-diphenyloxazol-2- yl)sulfanylethanol |
| A-61 | | 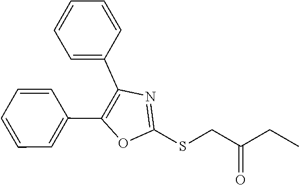 [SMILES: CCC(=O)CSc1nc(-c2ccccc2)c(-c2ccccc2)o1] | 1-(4,5-diphenyloxazol-2- yl)sulfanylbutan-2-one |
| A-62 | | 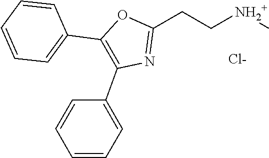 [SMILES: C[NH2+]CCc1nc(-c2ccccc2)c(-c2ccccc2)o1 ; [Cl-]] | 2-(4,5-diphenyloxazol-2- yl)ethyl-methyl-ammo- nium; chloride |
| A-63 | | 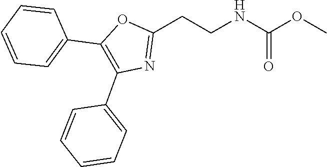 [SMILES: COC(=O)NCCc1nc(-c2ccccc2)c(-c2ccccc2)o1] | methyl N-[2-(4,5-diphen- yloxazol-2-yl)ethyl] carbamate |
| A-64 | | 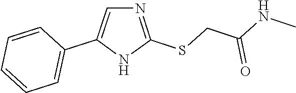 [SMILES: CNC(=O)CSc1ncc(-c2ccccc2)[nH]1] | N-methyl-2-[(5-phenyl- 1H-imidazol-2-yl)sul- fanyl]acetamide |
| A-65 | | 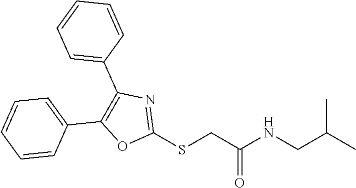 [SMILES: CC(C)CNC(=O)CSc1nc(-c2ccccc2)c(-c2ccccc2)o1] | 2-(4,5-diphenyloxazol-2- yl)sulfanyl-N-isobutyl- acetamide |
| A-66 | | 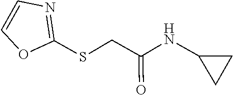 [SMILES: O=C(CSc1ncco1)NC1CC1] | N-cyclopropyl-2-oxazol- 2-ylsulfanyl-acetamide |
| A-67 | | 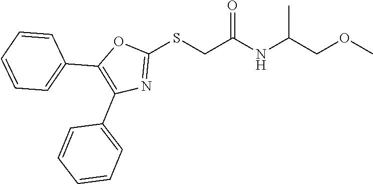 [SMILES: COCC(C)NC(=O)CSc1nc(-c2ccccc2)c(-c2ccccc2)o1] | 2-(4,5-diphenyloxazol-2- yl)sulfanyl-N-(2-methoxy- 1-methyl-ethyl)acetamide |
| A-68 | | 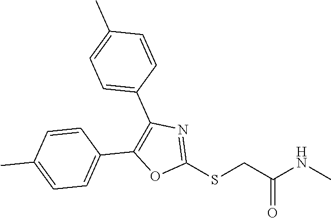 | 2-[4,5-bis(p-tolyl)oxazol- 2-yl]sulfanyl-N-methyl- acetamide |
| A-69 | | 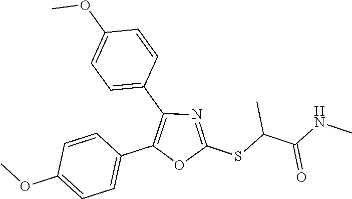 | 2-[4,5-bis(4-methoxy- phenyl)oxazol-2-yl]sul- fanyl-N-methyl-propan- amide |
| A-70 | | 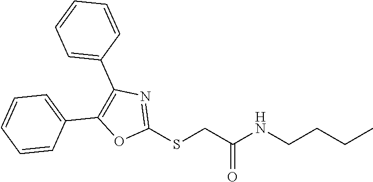 [SMILES: CCCCNC(=O)CSc1nc(-c2ccccc2)c(-c2ccccc2)o1] | N-butyl-2-(4,5-diphenyl- oxazol-2-yl)sulfanyl- acetamide |
| A-71 | | 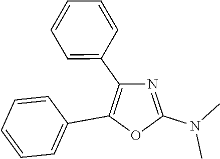 [SMILES: CN(C)c1nc(-c2ccccc2)c(-c2ccccc2)o1] | N,N-dimethyl-4,5-diphen- yl-oxazol-2-amine |
| A-72 | | 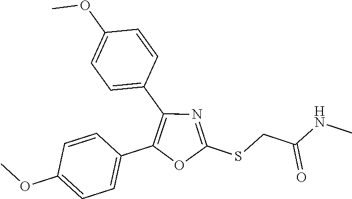 | 2-[4,5-bis(4-methoxyphen- yl)oxazol-2-yl]sulfanyl-N- methyl-acetamide |
| A-73 | | 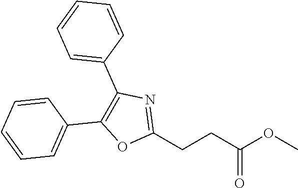 [SMILES: COC(=O)CCc1nc(-c2ccccc2)c(-c2ccccc2)o1] | methyl 3-(4,5-diphenyl- oxazol-2-yl)propanoate |

The EC50 value describes the concentration of cooling substance required for half-maximum effect and is therefore a measure of the potency of an agonistic pharmaceutical (potency of a pharmaceutical depending on the dose or concentration), whereby the potency corresponds to the reciprocal value of the EC50. Consequently, a low EC50 value corresponds to a high drug potency.

Thus, it can be seen from Tables 7 and 8 above that the compounds according to the invention described herein have excellent cooling properties and can cause intensive cooling effects even at low concentrations and are generally well below the EC50 reference value of 1.72 μM for the substance WS-3.

As Table 7 above shows, those structures of the general formulae (I), (II), (V) and (VI) in which R1 and R2 each represent a phenyl group, X represents an S atom, Y represents a methylene group or a methylene group substituted by a methyl or ethyl group, and Z represents —NH-cyclopropyl, —NH—CH₃, —N(CH₃)₂ or an azetidine have proved to be particularly advantageous. Furthermore, it could be observed that in the said structures m and n each stand for 1.

As Table 8 above shows, those structures of the general formulae (III), (IV), (VII) and (VIII) in which R1 and R2 each represent a phenyl group have proved to be particularly advantageous, X represents an S atom or a cyclopropyl group or a CH₂ group or piperidine, Y represents a methylene group or a methylene group which is substituted by a methyl group, and Z represents —NH—CH₃, —NH—CH₂—CH₃, —NH-cyclopropyl or —C—S—CH₃. Furthermore, it was observed that in the said structures m stands for 0 or 1 or n stands for 1.

Accordingly, compounds B-01, B-02, B-03, B-04, B-05, B-06 and B-07 with an EC50 value s 1.0 μM and compounds A-01, A-02, A-03, A-04, A-06, A-07 and A-08 with an EC50 value s 1.0 μM are particularly preferred with regard to the EC50 values.

Particularly efficient cooling effects with regard to TRPM8 activity and EC50 values can be observed for compounds B-01, B-02, B-03, B-04, B-05, B-06 and B-07 as well as for compounds A-01, A-02, A-03, A-04, A-06, A-07 and A-08 (TRPM8 activity 100% and EC value of s 1.0 μM).

In addition to the TRPM8 activity and active ingredient potency (EC50 value) described above, the compounds according to the invention also exhibit an intensive cooling effect.

In order to quantify the cooling effect, comparative tests are carried out using menthane-3-carboxylic acid-N-ethylamide as a reference. For these comparative tests, the skilled person replaces the compound or compounds to be used according to the invention with menthane-3-carboxylic acid-N-ethylamide (also referred to as WS-3). Then the intensities of the cooling effects of the respective compounds or active ingredients are sensory evaluated by trained panelists (n=10 to 11) as described below and compared with each other.

The cooling intensity was tested as follows: Test solutions with 5 ppm of the compounds according to the invention were each tasted in a 5% sugar solution as well as a corresponding solution with 30 ppm of the reference substance WS-3. This concentration for WS-3 was chosen because it has been shown that WS-3 exhibits good cooling effects at such concentrations. The panelists tasted the corresponding test solutions for exactly 40 seconds, rinsing the entire mouth with the corresponding test solution and then spitting out the sample or reference solution. Following the tasting, the test subjects rated the respective cooling intensity after one minute according to a scale of 1 (very weak) to 9 (very strong).

The results of the sensory tasting of exemplary selected compounds according to the invention are shown in the following Table 9 and Table 10.

TABLE 9
Results of the sensory tasting of exemplary coolants

| | Sensory | | |
|---|---|---|---|
| Compound | evaluation | Structure | IUPAC name |
| B-01 | 4.2 | 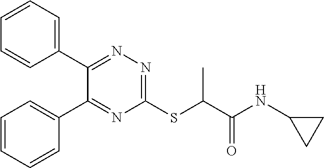 [SMILES: CC(Sc1nnc(-c2ccccc2)c(-c2ccccc2)n1)C(=O)NC1CC1] | N-Cyclopropyl-2-[(5,6- diphenyl-1,2,4-triazin-3- yl)sulfanyl]propenamide |
| B-02 | 4.1 | 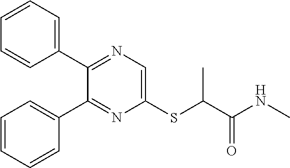 [SMILES: CNC(=O)C(C)Sc1cnc(-c2ccccc2)c(-c2ccccc2)n1] | 2-(5,6-diphenylpyrazin-2- yl)sulfanyl-N-methyl- propanamide |
| B-06 | 3.0 | 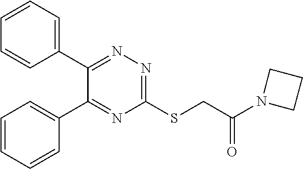 [SMILES: O=C(CSc1nnc(-c2ccccc2)c(-c2ccccc2)n1)N1CCC1] | 1-(azetidin-1-yl)-2-[(5,6- diphenyl-1,2,4-triazin-3- yl)sulfanyl]ethanone |
| B-07 | 0.75 | 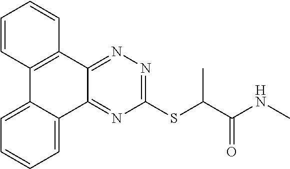 [SMILES: CNC(=O)C(C)Sc1nnc2c3ccccc3c3ccccc3c2n1] | N-methyl-2- phenanthro[9,10- e][1,2,4]triazine-3- ylsulfanyl-propanamide |
| B-11 | 5.3 | 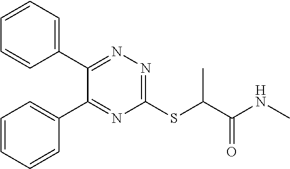 [SMILES: CNC(=O)C(C)Sc1nnc(-c2ccccc2)c(-c2ccccc2)n1] | 2-[(5,6-diphenyl-1,2,4- triazin-3-yl)sulfanyl]-N- methyl-propanamide |
| B-19 | 1.9 | 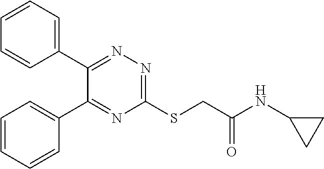 [SMILES: O=C(CSc1nnc(-c2ccccc2)c(-c2ccccc2)n1)NC1CC1] | N-cyclopropyl-2-[(5,6- diphenyl-1,2,4-triazin-3- yl)sulfanyl]acetamide |

TABLE 10
Results of the sensory tasting of exemplary coolants

| | Sensory | | |
|---|---|---|---|
| Compound | evaluation | Structure | IUPAC name |
| A-02 | 5.4 | 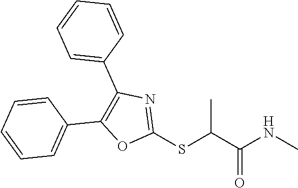 [SMILES: CNC(=O)C(C)Sc1nc(-c2ccccc2)c(-c2ccccc2)o1] | 2-(4,5-diphenyloxazol-2- yl)sulfanyl-N-methyl- propanamide |
| A-04 | 0.5 | 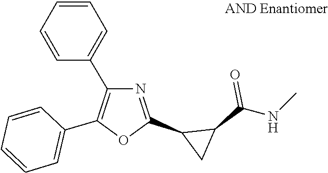 [SMILES: CNC(=O)[C@H]1C[C@H]1c1nc(-c2ccccc2)c(-c2ccccc2)o1] | rac-(1S,2R)-2-(4,5- diphenyloxazol-2-yl)-N- methyl- cyclopropanecarboxamides |
| A-05 | 1.1 | 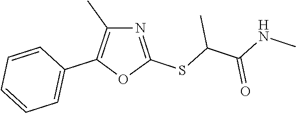 [SMILES: CNC(=O)C(C)Sc1nc(C)c(-c2ccccc2)o1] | N-methyl-2-(4-methyl-5- phenyl-oxazol-2- yl)sulfanyl-propanamide |
| A-07 | 2.67 | 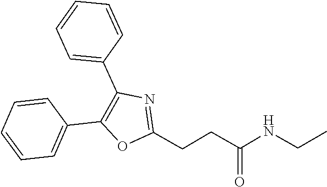 [SMILES: CCNC(=O)CCc1nc(-c2ccccc2)c(-c2ccccc2)o1] | 3-(4,5-diphenyloxazol-2- yl)-N-ethyl-propanamide |
| A-09 | 4.66 | 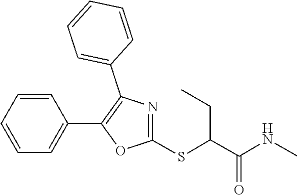 [SMILES: CCC(Sc1nc(-c2ccccc2)c(-c2ccccc2)o1)C(=O)NC] | 2-(4,5-diphenyloxazol-2- yl)sulfanyl-N-methyl- butanamide |
| A-10 | 5.38 | 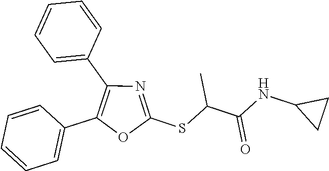 [SMILES: CC(Sc1nc(-c2ccccc2)c(-c2ccccc2)o1)C(=O)NC1CC1] | N-cyclopropyl-2-(4,5- diphenyloxazol-2- yl)sulfanyl-propanamide |
| A-12 | 1.26 | 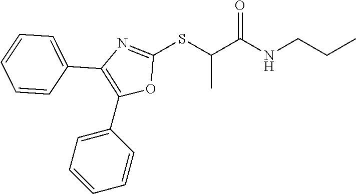 [SMILES: CCCNC(=O)C(C)Sc1nc(-c2ccccc2)c(-c2ccccc2)o1] | 2-(4,5-diphenyloxazol-2- yl)sulfanyl-N-propyl- propanamide |
| A-16 | 2.3 | 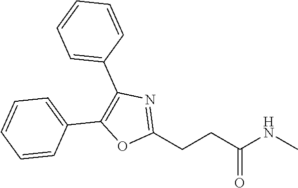 [SMILES: CNC(=O)CCc1nc(-c2ccccc2)c(-c2ccccc2)o1] | 3-(4,5-diphenyloxazol-2- yl)-N-methyl-propanamide |

Surprisingly, it has been shown that the compounds described herein produce a noticeably more intense or comparable cooling effect compared to the WS-3 reference sample. In particular, it has been shown that the reference sample containing WS-3 showed a cooling intensity of about 5.4 in the sensory evaluation, while the cooling intensity of the substances according to the invention, such as compound B-01 with 4.2, compound B-02 with 4.1, compound B-11 with 5.3, compound A-02 with 5.4, compound A-09 with 4.66, and compound A-10 with 5.38.

It should also be noted that WS-3 is capable of producing noticeably lower cooling intensities despite a six-fold higher concentration. Conversely, significantly lower concentrations of the compounds according to the invention are necessary in order to produce significantly more intensive cooling effects than conventional cooling substances (such as WS-3). This shows that the compounds according to the invention produce an intensive and thus highly effective cooling effect even when used in low concentrations and that only very small amounts need to be used in corresponding final formulations, such as product formulations containing these cooling substances, in order to produce cooling effects that are perceived as intensive.

In this context, it is preferred that in the corresponding comparisons the cooling effect of the samples with the compound(s) to be used according to the invention is preferably extended by at least 10 minutes, preferably by at least 15 minutes, more preferably by at least 20 minutes, even more preferably by at least 30 minutes, particularly preferably by at least 60 minutes, most preferably by at least 90 minutes compared to the reference samples containing WS-3.

### Examples of Synthesis

Preparation of the compounds according to the invention

### Method A:

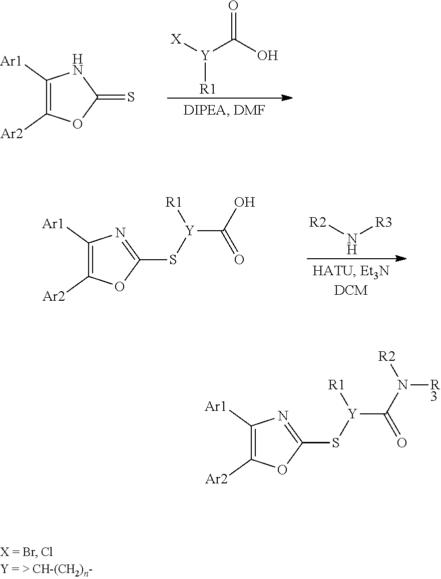

The 2-oxazolethiol derivative (1 eq.) and the bromocarboxylic acid or chlorocarboxylic acid (1.2 eq.) were dissolved in dry DMF and N,N-diisopropylethylamine (1.5 eq.) was added at room temperature. The reaction solution was stirred at room temperature until the uHPLC control indicated complete conversion. The reaction mixture was then diluted with water and acidified with 1M hydrochloric acid to approx. pH 3. It was extracted with DCM and the combined organic phases were washed with water and filtered through a phase separator. After concentration of the organic phase, the 2-oxazolecarboxylic acid derivative was obtained in a satisfactory purity to be used in the next reaction step.

The 2-oxazolecarboxylic acid derivative (1 eq.) obtained from method A and the required amine (if necessary as THF solution) (3 eq.) were dissolved in ethyl acetate and HATU (1.5 eq.) followed by N,N-diisopropylethylamine (5 eq.) were added at room temperature; more DIPEA was added dropwise until the pH was adjusted to approx. 9. The reaction solution was stirred at room temperature until the uHPLC control indicated complete conversion. It was then diluted with saturated NaHCO₃ solution and extracted with DCM. The combined organic phases were filtered through a phase separator. After concentration of the organic phase, the crude product was purified by column chromatography and the desired product was obtained as oil or solid.

### Method B:

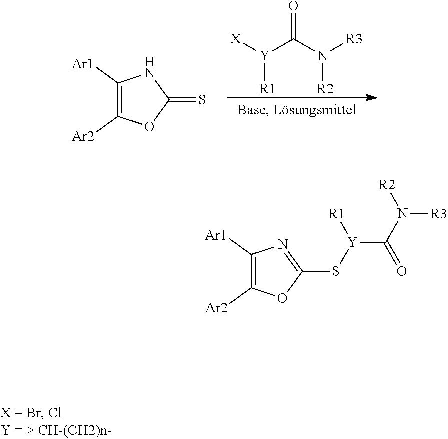

The 2-oxazolethiol derivative (1 eq.) and the bromamide or chloramide (1.2 eq.) were either dissolved in dry acetone and K₂ CO₃ (2 eq.) was added at room temperature or dissolved in dry DMF and diisopropylethylamine (1.5 eq.) was added at room temperature. The reaction solution was heated to 40° C. and stirred until the uHPLC control indicated complete conversion. It was then diluted with water and extracted with either EtOAc or DCM. The combined organic phases were washed with water and saturated NaCl solution and dried over MgSO₄. After concentration of the organic phase, the crude product was purified by column chromatography and the desired product was obtained as oil or solid.

### Method C:

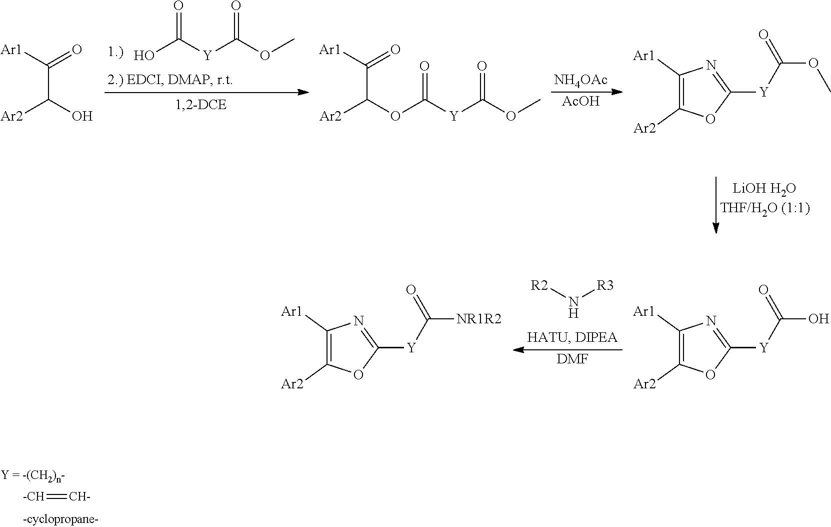

The carboxylic acid derivative (1 eq) was dissolved in dry DCE and cooled to 0-5° C. The hydrochloride of 1-ethyl-3-(3-dimethylaminopropyl)carbo-diimide (1.1 eq) was added and stirred for 30 min. Benzoin (1 eq) was then added, followed by DMAP (1.2 eq). The reaction mixture was warmed to room temperature and stirred for 24 hours. The mixture was then diluted with DCM and filtered first three times with 10 wt.-% citric acid solution, then with saturated NaHCO₃ solution and through a phase separator. After concentration of the organic phase, the ester was obtained in a satisfactory purity to be used in the next reaction step. If it was a coolant, the product was purified by column chromatography and the desired product was obtained as an oil or solid.

The ester (1 eq) and ammonium acetate (5 eq) were dissolved in acetic acid and stirred under reflux for 90 min. After cooling to room temperature, the reaction mixture was added to water and extracted with dichloromethane. The combined organic phase was filtered through a phase separator and concentrated. The product could be used in the next reaction step without further purification. If it was a coolant, the product was purified by column chromatography and the desired product was obtained as an oil or solid.

The oxazole derivative (1 eq) was dissolved in a mixture of THF and water (1:1) and cooled to 0° C. LiOH monohydrate (2 eq) was then added. LiOH monohydrate (2 eq) was then added and slowly warmed to room temperature and stirred until complete conversion was detected. The reaction solution was then diluted with water and acidified with 10 wt % citric acid to a pH of 3. The aqueous solution was extracted with DCM. The combined organic phases were dried over MgSO₄ and concentrated. The carboxylic acid derivative could be used in the next reaction step without further purification. If it was a coolant, the product was purified by column chromatography and the desired product was obtained as oil or solid.

The carboxylic acid derivative (1 eq) and the required amine (if necessary as a THF solution) (1 eq) were dissolved in DMF and cooled to 0° C. HATU (1 eq) was then added followed by N,N-diisopropylethylamine (3 eq). HATU (1 eq.) was then added, followed by N,N-diisopropylethylamine (3 eq.). The reaction solution was stirred at room temperature until the uHPLC control indicated complete conversion. It was then diluted with water and extracted with DCM. The combined organic phases were washed with water, three times with 10 wt % citric acid and finally with saturated NaHCO₃ and filtered through a phase separator. The solvent was removed under vacuum. The crude product was purified by column chromatography and the desired product was obtained as oil or solid.

### Method D:

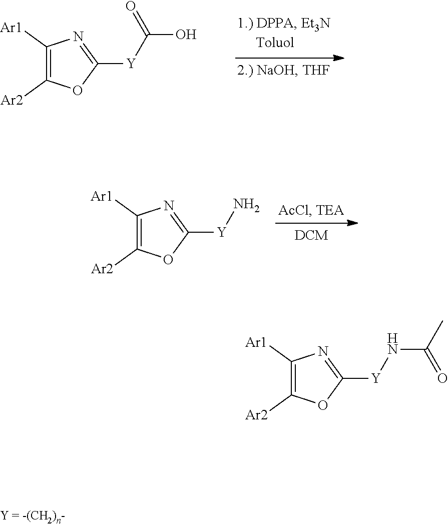

Diphenyl azidophosphate (1.1 eq) and triethylamine (1.1 eq) were added to a solution of oxaprozine derivative (1 eq) in dry toluene and stirred at room temperature. The course of the reaction was monitored by HPLC. After complete conversion, the reaction was diluted with toluene and washed with water. The organic phase was dried with MgSO₄ and concentrated in vacuo. The crude product was dissolved in MeOH and stirred at 75° C. for 24 hours, and then concentrated in vacuo. The crude product was purified by column chromatography (reversed phase) and the desired product was obtained as oil or solid. The carbamate (1 eq) was dissolved in THF/MeOH (2:1) and 2M NaOH solution (16 eq) was added. The reaction mixture was heated to 70° C. and stirred until the HPLC control showed complete conversion. The reaction mixture was then cooled to room temperature and diluted with water. It was extracted three times with a mixture of chloroform and isopropanol (7:3) and the combined organic phases were filtered through a phase separator and concentrated. The product could be used in the next reaction step without further purification. If it was a coolant, the product was purified by column chromatography and the desired product was obtained as an oil or solid.

The amine derivative (1 eq) was dissolved in dry DCM and triethylamine (2 eq) followed by acetyl chloride (1.2 eq) was added. The reaction mixture was stirred at room temperature until the HPLC control showed complete conversion. The reaction mixture was diluted with DCM and washed with saturated NaHCO₃ solution. The organic phase was filtered through a phase separator and concentrated in vacuo. The crude product was purified by column chromatography and the desired product was obtained as oil or solid.

### Method E:

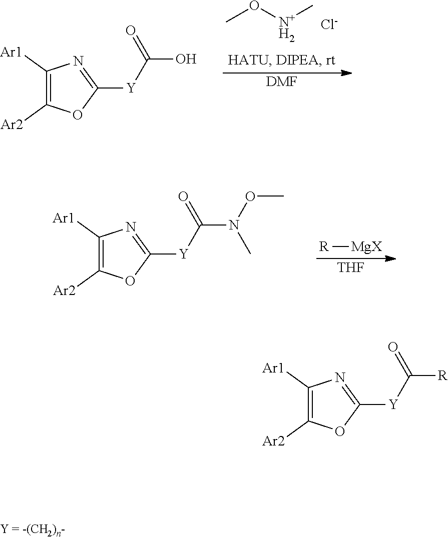

The oxazole carboxylic acid (1 eq) and the N,O-dimethylhydroxylamine hydrochloride (1 eq) were dissolved in DMF. HATU (1.5 eq) and diisopropylethylamine (5 eq) were added and the reaction mixture was stirred at room temperature until the HPLC control showed complete conversion. The reaction mixture was diluted with semi-saturated NaHCO₃ solution and extracted three times with DCM. The combined organic phases were washed with water and saturated NaCl solution, filtered through a phase separator and concentrated in vacuo. The crude product was purified by column chromatography and the desired product was obtained as oil or solid.

The Weinreb amide (1 eq) was dissolved in dry THF and cooled to 0° C. Grignard solution in THF was added slowly at 0° C. and after completion of the addition, the reaction mixture was stirred at room temperature until control by HPLC showed complete conversion. The reaction was quenched by addition of saturated NH4Cl solution and the aqueous phase was extracted three times with EtOAc. The combined organic phases were dried over MgSO₄ and concentrated in vacuo. The crude product was purified by column chromatography and the desired product was obtained as oil or solid.

### Method F:

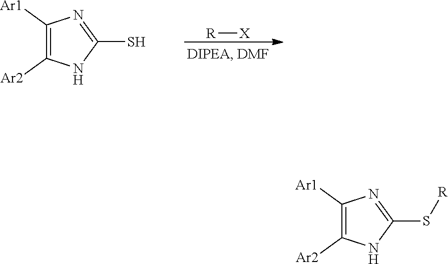

The 2-imidazolethiol derivative (1 eq) was dissolved in dry dimethylformamide and the corresponding organohalogen compound (1.2 eq) followed by diisopropylethylamine (1.5 eq) was added. The reaction mixture was stirred at room temperature until the HPLC control showed complete conversion. The reaction mixture was diluted with water and extracted with DCM. The combined organic phases were dried over MgSO₄ and concentrated in vacuo. The crude product was purified by column chromatography and the desired product was obtained as oil or solid.

### Method G:

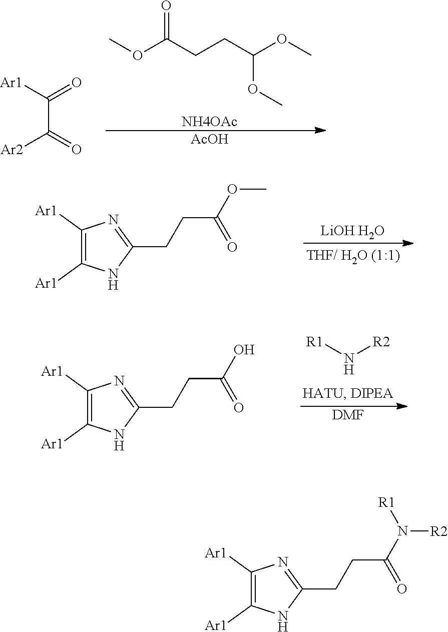

The benzil derivative (1 eq), dimethoxy ester (1.3 eq) and ammonium acetate (7 eq) were dissolved in acetic acid and stirred under reflux in a nitrogen atmosphere until the HPLC control showed complete conversion. The reaction mixture was then cooled to room temperature and added to water. It was made basic with 25 wt. % ammonia solution and extracted with DCM. The combined organic phases were dried over MgSO₄ and concentrated in vacuo. The product could be used in the next reaction step without further purification. The final product was purified by column chromatography and the desired product was obtained as oil or solid. The corresponding carboxylic acids and the corresponding amides were prepared as described in method D.

### Method H:

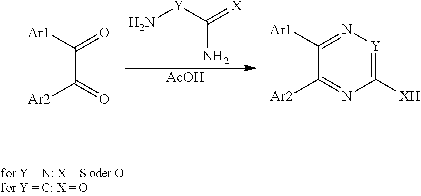

For pyrazines: The benzil derivative (1 eq) was dissolved in methanol and glycinamide hydrochloride (1.1 eq) followed by NaOH (2 eq) was added and stirred at 70° C. for 4 hours. The reaction mixture was then cooled to room temperature, 2M hydrochloric acid was added and stirred for 30 minutes. Saturated NaHCO3 solution was then added, the solid was filtered off and washed with methanol. The crude product was purified by column chromatography and the desired product was obtained as oil or solid.

For 1,2,4-triazines: The benzil derivative (1 eq) was dissolved in acetic acid and heated to 100° C. Thiosemicarbazide (2 eq) or corresponding semicarbazide hydrochloride (1 eq) was added. Thiosemicarbazide (2 eq) or correspondingly semicarbazide hydrochloride (1 eq) was added and stirred under reflux for 6 hours. The reaction mixture was then cooled to 5° C. and the resulting solid was filtered off and washed with water. After drying in a vacuum, it could be used in the next reaction step without further purification.

### Method I:

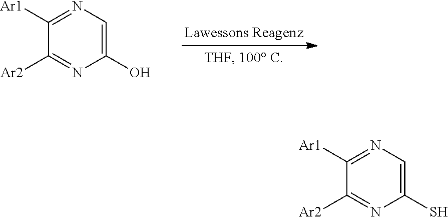

A suspension of Lawessons reagent (2 eq) and pyrazine alcohol (1 eq) in THF was stirred under reflux overnight. The reaction mixture was then cooled to room temperature and diluted with water. It was extracted with diethyl ether and the combined organic phases were dried over Na₂SO₄, filtered and concentrated in vacuo. The crude product was purified by column chromatography and the desired product was obtained as oil or solid.

### Method J:

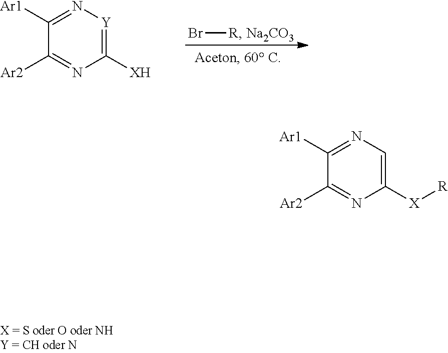

The thiol or alcohol or amine derivative (1 eq) was dissolved in acetone and Na₂CO₃ (1.5 to 6 eq) was added. The bromine compound (1.1 to 1.5 eq) was added to the suspension and stirred under reflux until reaction control via DC showed complete conversion. The solid was filtered off and washed with acetone. The filtrate was coevaporated with silica. After purification by column chromatography, the desired product was obtained as oil or solid.

### Method K:

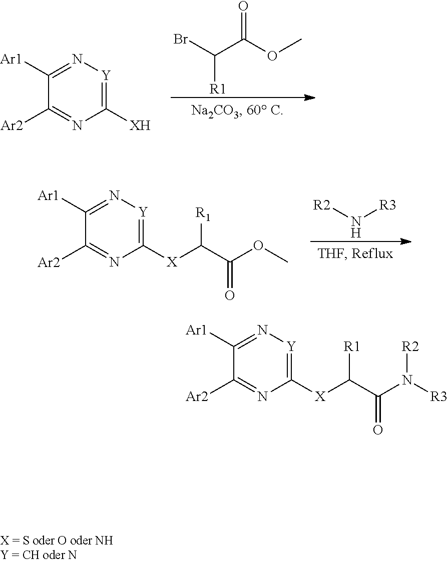

The thiol or alcohol or amine derivative (1 eq) was dissolved in acetone and Na₂ CO₃ (1.5 to 6 eq) was added. The 2-bromomethyl ester (1 eq) was added to the suspension and stirred at 60° C. until reaction control via DC showed complete conversion. The reaction mixture was then cooled to room temperature, filtered and washed with acetone. The filtrate was concentrated and the crude product obtained was purified by column chromatography to obtain the desired methyl ester as an oil or solid. The methyl ester (1 eq) was stirred in a 2M solution of the amine (10 eq) in THF at 80° C. overnight. The reaction solution was then concentrated in vacuo. The crude product was purified by column chromatography to obtain the desired amide as oil or solid.

### Method L and L′:

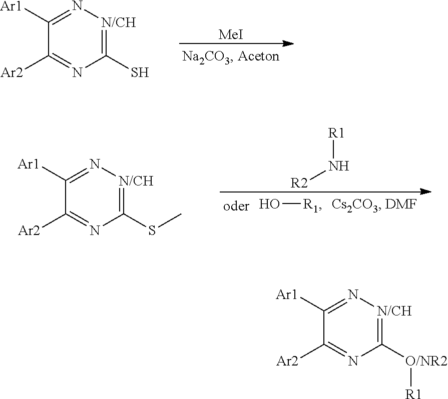

The thiol (1 eq) was dissolved in acetone and Na₂CO₃ (1.5 eq) was added, followed by methyl iodide (1 eq). The reaction mixture was stirred at 60° C. until control of DC showed complete conversion. The reaction mixture was then filtered and washed with acetone. The filtrate was concentrated. The product could be used in the next reaction step without further purification. The final product was purified by column chromatography and the desired product was obtained as an oil or solid. The methyl thioether (1 eq) and the required amine (10 eq) were stirred at elevated temperature (up to 120° C.) until a control of DC showed complete conversion. The reaction mixture was then concentrated in vacuo and the crude product obtained was purified by column chromatography. The methyl thioether (1 eq) and the required alcohol (1 eq) were dissolved in dry DMF and Cs₂CO₃ (1 eq) was added. The reaction mixture was stirred at 100° C. until a control of DC showed complete conversion. The reaction mixture was then filtered off and concentrated in vacuo. The crude product obtained was purified by column chromatography.

### Method M:

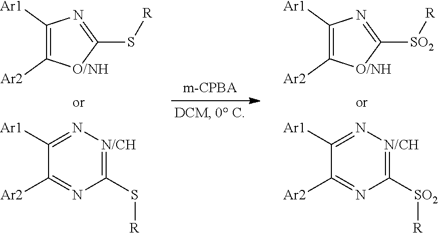

The thioether (1 eq) was dissolved in dry DCM and cooled to 0° C. 3-chloroperbenzoic acid (3 eq) was added and the reaction solution was warmed to room temperature and stirred overnight. The reaction was then quenched with 5 wt.-% aqueous sodium disulfite solution and stirred for one hour. The phases were separated and the organic phase was washed with water, filtered through a phase separator and concentrated in vacuo. The crude product was purified by column chromatography and the desired product was obtained as oil or solid.

Table 11 below lists the analytical data for identifying the synthesized compounds according to the present invention. In the values, the decimal point is represented by a dot.

TABLE 11
1H-NMR and 13C-NMR data of coolants according to the invention (decimal point)

| Compound | 1H-NMR | 13C-NMR |
|---|---|---|
| A-01 | 1H NMR (400 MHz, DMSO) δ 8.23 (q, J = | 13C NMR (101 MHz, DMSO) δ |
| | 4.7 Hz, 1H), 7.61-7.57 (m, 2H), 7.55-7.50 | 174.55, 171.17, 170.39, 157.65, |
| | (m, 2H), 7.40 (dtd, J = 9.8, 6.5, 2.7 Hz, 7H), | 146.61, 135.70, 131.40, 128.64, |
| | 4.45 (q, J = 7.0 Hz, 1H), 2.77 (d, J = 15.4 | 128.39, 128.18, 127.82, 127.34, |
| | Hz, 2H), 2.68 (d, J = 4.6 Hz, 3H), 2.67 (d, J = | 125.99, 72.33, 45.96, 42.67, 25.79, |
| | 15.4 Hz, 3H), 1.62 (d, J = 7.0 Hz, 3H) | 19.35 |
| A-02 | 1H NMR (400 MHz, CDCl3) δ 7.63-7.60 | 13C NMR (101 MHz, CDCl3) δ |
| | (m, 2H), 7.56-7.53 (m, 2H), 7.42-7.31 (m, | 171.82, 159.02, 147.65, 135.96, |
| | 6H), 4.30 (q, J = 7.35 Hz, 1H), 2.84 (d, 4.84 | 131.70, 128.85, 128.75, 128.72, |
| | Hz, 3H), 1.65 (d, J = 7.45 Hz), 3H) | 128.51, 128.21, 127.71, 126.43, |
| | | 43.87, 26.62, 17.01 |
| A-03 | 1H NMR (400 MHz, CDCl3) δ 7.64-7.59 | 13C NMR (101 MHz, CDCl3) δ |
| | (m, 2H), 7.58-7.52 (m, 2H), 7.43-7.32 (m, | 171.96, 159.20, 147.81, 136.05, |
| | 6H), 6.71-6.52 (m, 1H), 4.34 (q, J = 7.4 Hz, | 131.77, 129.01, 128.89, 128.86, |
| | 1H), 2.84 (s, 3H), 1.65 (d, J = 7.3 Hz, 3H) | 128.67, 128.32, 127.86, 126.57, |
| | | 44.01, 26.76, 17.15 |
| A-04 | 1H NMR (400 MHz, CDCl3) δ 7.63-7.59 | 13C NMR (101 MHz, CDCl3) δ |
| | (m, 2H), 7.59-7.56 (m, 2H), 7.38-7.27 (m, | 169.87, 160.51, 145.63, 135.22, |
| | 6H), 6.36-6.25 (m, 1H), 2.73 (d, J = 4.8 Hz, | 132.53, 129.01, 128.73, 128.66, |
| | 3H), 2.51 (td, J = 8.8, 6.8 Hz, 1H), 2.09 (td, J = | 128.58, 128.21, 128.02, 126.68, |
| | 8.8, 6.8 Hz, 1H), 1.92 (td, J = 6.8, 5.2 Hz, | 26.59, 24.26, 16.71, 12.04 |
| | 1H), 1.51 (td, J = 8.8, 5.3 Hz, 1H) | |
| A-05 | 1H NMR (400 MHz, CDCl3) δ 7.56-7.52 | 13C NMR (101 MHz, CDCl3) δ |
| | (m, 2H), 7.47-7.40 (m, 2H), 7.34-7.28 (m, | 171.98, 157.70, 147.84, 132.88, |
| | 2H), 4.22 (q, J = 7.4 Hz, 1H), 2.83 (d, J = | 128.97, 128.57, 127.96, 125.22, |
| | 4.8 Hz, 3H), 2.41 (s, 3H), 1.62 (d, J = 7.4 | 44.03, 26.70, 17.23, 13.46 |
| | Hz, 3H) | |
| A-06 | 1H NMR (400 MHz, CDCl3) δ 7.67 (s, 1H), | 13C NMR (101 MHz, CDCl3) δ |
| | 7.64-7.58 (m, 2H), 7.58-7.50 (m, 2H), | 169.55, 159.19, 148.12, 136.01, |
| | 7.44-7.31 (m, 6H), 3.78 (s, 2H), 2.80-2.70 | 131.55, 128.94, 128.78, 128.73, |
| | (m, 1H), 0.75 (td, J = 7.0, 5.3 Hz, 2H), 0.50- | 128.59, 128.14, 127.65, 126.44, |
| | 0.44 (m, 2H) | 35.24, 22.86, 6.43 |
| A-07 | 1H NMR (400 MHz, CDCl3) δ 7.65-7.58 | 13C NMR (101 MHz, CDCl3) δ |
| | (m, 2H), 7.59-7.51 (m, 2H), 7.41-7.28 (m, | 171.09, 162.46, 145.47, 134.96, |
| | 6H), 6.02 (s, 1H), 3.35-3.27 (m, 2H), 3.20 | 132.47, 128.90, 128.66, 128.59, |
| | (t, J = 7.3 Hz, 2H), 2.74 (t, J = 7.3 Hz, 2H), | 128.50, 128.11, 127.88, 126.47, |
| | 1.13 (t, J = 7.3 Hz, 3H) | 34.51, 33.17, 24.09, 14.82 |
| A-08 | 1H NMR (400 MHz, CDCl3) δ 7.83 (s, 1H), | 13C NMR (101 MHz, CDCl3) δ |
| | 7.81 (s, 1H), 7.61 (dd, J = 8.2, 2.0 Hz, 4H), | 168.60, 168.43, 162.39, 162.23, |
| | 7.19 (d, J = 7.8 Hz, 4H), 6.02 (d, J = 5.3 Hz, | 141.09, 140.80, 138.08, 137.81, |
| | 1H), 5.38-5.34 (m, 1H), 4.57 (dd, J = 13.7, | 133.30, 133.15, 129.43, 129.36, |
| | 3.9 Hz, 1H), 3.81 (dd, J = 14.0, 3.3 Hz, 1H), | 128.28, 127.93, 125.48, 52.12, |
| | 3.53 (d, J = 13.7 Hz, 1H), 3.46-3.39 (m, | 47.30, 44.14, 39.11, 35.62, 35.40, |
| | 3H), 3.37 (td, J = 13.7, 3.3 Hz, 1H), 2.75 (td, | 27.88, 27.36, 25.59, 24.91, 21.30, |
| | J = 13.3, 2.6 Hz, 1H), 2.45 (dt, J = 12.9, 2.4 | 19.98, 15.62, 15.51 |
| | Hz, 1H), 2.37 (s, 6H), 2.25 (s, 3H), 2.22 (s, | |
| | 3H), 2.04-1.94 (m, 1H), 1.90-1.78 (m, | |
| | 2H), 1.78-1.67 (m, 4H), 1.72-1.45 (m, 4H) | |
| A-09 | 1H NMR (400 MHz, DMSO) δ 8.33 (q, J = | 13C NMR (101 MHz, DMSO) δ |
| | 4.6 Hz, 1H), 7.61-7.55 (m, 2H), 7.55-7.50 | 169.51, 157.62, 146.67, 135.70, |
| | (m, 2H), 7.48-7.43 (m, 2H), 7.43 (dt, J = | 131.30, 128.96, 128.92, 128.64, |
| | 5.7, 1.0 Hz, 2H), 7.42-7.39 (m, 1H), 7.39 | 128.43, 127.73, 127.36, 126.13, |
| | (d, J = 7.2 Hz, 1H), 4.31 (dd, J = 7.7, 6.2 Hz, | 52.18, 26.33, 25.73, 11.19 |
| | 1H), 2.67-2.64 (m, 3H), 2.06-1.86 (m, | |
| | 2H), 0.97 (t, J = 7.4 Hz, 3H) | |
| A-10 | 1H NMR (400 MHz, chloroform-d) δ 7.71- | 13C NMR (101 MHz, CDCl3) δ |
| | 7.64 (m, 1H), 7.63-7.59 (m, 2H), 7.57- | 172.51, 159.16, 147.62, 135.84, |
| | 7.53 (m, 2H), 7.43-7.33 (m, 6H), 4.21 (q, J = | 131.62, 128.89, 128.76, 128.72, |
| | 7.3 Hz, 1H), 2.80-2.72 (m, 1H), 1.62 (d, J = | 128.55, 128.16, 127.65, 126.43, |
| | 7.4 Hz, 3H), 0.76-0.71 (m, 2H), 0.46- | 43.67, 22.84, 16.66, 6.58, 6.19 |
| | 0.41 (m, 2H) | |
| A-11 | 1H NMR (400 MHz, DMSO) δ 7.46-7.38 | 13C NMR (101 MHz, DMSO) δ |
| | (m, 4H), 7.38-7.29 (m, 4H), 7.29-7.20 (m, | 140.44, 128.34, 127.23, 58.67, |
| | 2H), 3.24 (t, J = 6.9 Hz, 2H), 2.59 (t, J = 6.9 | 44.78, 30.53 |
| | Hz, 2H), 2.19 (s, 6H) | |
| A-12 | 1H NMR (400 MHz, CDCl3) δ 7.64-7.59 | 13C NMR (101 MHz, CDCl3) δ |
| | (m, 2H), 7.57-7.53 (m, 2H), 7.42-7.32 (m, | 171.12, 159.12, 147.61, 135.95, |
| | 6H), 4.30 (q, J = 7.3 Hz, 1H), 3.26-3.19 (m, | 131.69, 128.85, 128.75, 128.67, |
| | 2H), 1.64 (d, J = 7.4 Hz, 3H), 1.51-1.41 (m, | 128.50, 128.21, 127.72, 126.42, |
| | 2H), 0.82 (t, J = 7.4 Hz, 3H) | 43.97, 41.52, 22.56, 16.89, 11.29 |
| A-15 | 1H NMR (400 MHz, chloroform-d) δ 7.63- | 13C NMR (101 MHz, CDCl3) δ |
| | 7.59 (m, 2H), 7.57-7.52 (m, 2H), 7.42- | 168.66, 158.92, 147.97, 136.09, |
| | 7.31 (m, 7H), 3.86 (s, 2H), 2.84 (d, J = 4.9 | 131.63, 128.90, 128.77, 128.73, |
| | Hz, 3H) | 128.55, 128.20, 127.72, 126.45, |
| | | 35.21, 26.69 |
| A-16 | 1H NMR (600 MHz, CDCl3) δ 7.62-7.60 | 13C NMR (151 MHz, CDCl3) δ |
| | (m, 2H), 7.58-7.55 (m, 2H), 7.40-7.30 (m, | 171.91, 162.42, 145.49, 134.94, |
| | 6H), 6.03 (s, 1H), 3.21 (t, J = 7.3 Hz, 2H), | 132.44, 128.87, 128.66, 128.60, |
| | 2.84 (d, J = 4.8 Hz, 3H), 2.76 (t, J = 7.3 Hz, | 128.50, 128.12, 127.90, 126.45, |
| | 2H) | 32.95, 26.44, 24.04 |
| A-17 | 1H NMR (400 MHz, CDCl3) δ 7.64-7.59 | 13C NMR (101 MHz, CDCl3) δ |
| | (m, 2H), 7.57-7.53 (m, 2H), 7.42-7.31 (m, | 167.85, 158.97, 147.92, 136.07, |
| | 6H), 3.83 (s, 2H), 3.31 (qd, J = 7.3, 5.7 Hz, | 131.63, 128.90, 128.77, 128.70, |
| | 2H), 1.11 (t, J = 7.3 Hz, 3H) | 128.54, 128.21, 127.69, 126.44, |
| | | 35.40, 34.84, 14.55 |
| A-18 | 1H NMR (600 MHz, CDCl3) δ 7.63-7.59 | 13C NMR (151 MHz, CDCl3) δ |
| | (m, 2H), 7.58-7.54 (m, 2H), 7.40-7.30 (m, | 172.73, 162.41, 145.49, 134.89, |
| | 6H), 6.28-6.21 (m, 1H), 3.19 (t, J = 7.2 Hz, | 132.39, 128.84, 128.67, 128.61, |
| | 2H), 2.77-2.71 (m, 1H), 2.71 (t, J = 7.2 Hz, | 128.53, 128.14, 127.85, 126.47, |
| | 2H), 0.79-0.72 (m, 2H), 0.51-0.42 (m, 2H) | 32.94, 24.00, 22.69, 6.57 |
| A-19 | 1H NMR (400 MHz, CDCl3) δ 7.67-7.60 | 13C NMR (101 MHz, CDCl3) δ |
| | (m, 2H), 7.58-7.52 (m, 2H), 7.42-7.31 (m, | 167.11, 159.03, 147.88, 136.04, |
| | 6H), 7.29-7.22 (m, 1H), 4.05 (dhept, J = | 131.62, 128.89, 128.76, 128.68, |
| | 7.9, 6.6 Hz, 1H), 3.79 (s, 2H), 1.11 (d, J = | 128.53, 128.20, 127.68, 126.43, |
| | 6.6 Hz, 6H) | 41.91, 35.58, 22.52 |
| A-20 | 1H NMR (400 MHz, DMSO) δ 7.78 (s, 1H), | 13C NMR (101 MHz, DMSO) δ |
| | 7.61-7.55 (m, 2H), 7.55-7.50 (m, 2H), | 171.91, 157.75, 146.60, 135.67, |
| | 7.49-7.36 (m, 6H), 7.33 (s, 1H), 4.46 (q, J = | 131.30, 128.95, 128.91, 128.62, |
| | 7.0 Hz, 1H), 1.61 (d, J = 7.0 Hz, 3H). | 128.41, 127.72, 127.36, 126.15, |
| | | 46.22, 19.45 |
| A-21 | 1H NMR (400 MHz, CDCl3) δ 7.68-7.61 | 13C NMR (101 MHz, CDCl3) δ |
| | (m, 4H), 7.60-7.52 (m, 4H), 7.40-7.28 (m, | 168.39, 160.90, 145.79, 135.27, |
| | 12H), 6.09 (d, J = 5.2 Hz, 1H), 5.43 (d, J = | 132.47, 128.95, 128.75, 128.60, |
| | 5.4 Hz, 1H), 4.61 (d, J = 13.8 Hz, 1H), 3.83 | 128.53, 128.07, 127.97, 126.64, |
| | (d, J = 13.6 Hz, 1H), 3.58 (d, J = 14.1 Hz, | 52.10, 47.34, 44.29, 39.20, 35.65, |
| | 1H), 3.47 (qd, J = 12.8, 2.6 Hz, 1H), 3.44- | 35.39, 27.94, 27.43, 25.63, 24.96, |
| | 3.40 (m, 2H), 3.38 (d, J = 13.7 Hz, 1H), 2.85 | 20.11, 15.60 |
| | (t, J = 13.3 Hz, 1H), 2.50 (dq, J = 13.2, 2.0 | |
| | Hz, 2H), 2.24 (s, 3H), 2.23 (s, 3H), 2.10- | |
| | 1.44 (m, 10H) | |
| A-22 | 1H NMR (600 MHz, CDCl3) δ 7.63-7.59 | 13C NMR (151 MHz, CDCl3) δ |
| | (m, 2H), 7.58-7.54 (m, 2H), 7.40-7.30 (m, | 172.73, 162.41, 145.49, 134.89, |
| | 6H), 6.28-6.21 (m, 1H), 3.19 (t, J = 7.2 Hz, | 132.39, 128.84, 128.67, 128.61, |
| | 2H), 2.77-2.71 (m, 1H), 2.71 (t, J = 7.2 Hz, | 128.53, 128.14, 127.85, 126.47, |
| | 2H), 0.79-0.72 (m, 2H), 0.51-0.42 (m, 2H) | 32.94, 24.00, 22.69, 6.57 |
| A-23 | 1H NMR (400 MHz, CDCl3) δ 7.67-7.63 | 13C NMR (101 MHz, CDCl3) δ |
| | (m, 2H), 7.58-7.53 (m, 2H), 7.40-7.30 (m, | 159.35, 147.05, 136.39, 132.20, |
| | 6H), 3.41 (t, J = 6.9 Hz, 2H), 2.77 (t, J = 7.0 | 128.74, 128.65, 128.56, 128.45, |
| | Hz, 2H), 2.34 (s, 6H) | 128.20, 127.87, 126.34, 58.39, |
| | | 45.22, 30.46 |
| A-24 | 1H NMR (400 MHz, CDCl3) δ 7.66-7.61 | 13C NMR (101 MHz, CDCl3) δ |
| | (m, 2H), 7.59-7.55 (m, 2H), 7.42-7.28 (m, | 170.19, 161.56, 145.68, 135.00, |
| | 6H), 6.56-6.30 (m, 1H), 3.77 (q, J = 6.1 Hz, | 132.35, 128.78, 128.71, 128.64, |
| | 2H), 3.04 (t, J = 6.1 Hz, 2H), 1.99 (s, 3H) | 128.63, 128.22, 127.90, 126.50, |
| | | 36.26, 28.27, 23.43 |
| A-25 | 1H NMR (400 MHz, CDCl3) δ 7.53-7.47 | 13C NMR (101 MHz, CDCl3) δ |
| | (m, 4H), 7.45-7.40 (m, 4H), 7.21-7.17 (m, | 171.90, 171.81, 158.49, 147.53, |
| | 4H), 7.17-7.13 (m, 4H), 4.28 (q, J = 7.3 Hz, | 138.81, 138.27, 135.43, 130.00, |
| | 2H), 2.83 (s, 3H), 2.82 (s, 3H), 2.38 (s, 6H), | 129.39, 129.35, 128.88, 127.51, |
| | 2.36 (s, 6H), 1.63 (d, J = 7.4 Hz, 6H) | 126.34, 125.67, 125.49, 43.81, |
| | | 43.76, 26.57, 26.43, 21.36, 21.33, |
| | | 16.98 |
| A-26 | 1H NMR (400 MHz, CDCl3) δ 7.65-7.59 | 13C NMR (101 MHz, CDCl3) δ |
| | (m, 2H), 7.59-7.51 (m, 2H), 7.42-7.33 (m, | 168.54, 158.88, 148.06, 136.13, |
| | 6H), 4.06 (hd, J = 6.9, 3.6 Hz, 1H), 3.85 (d, J = | 131.44, 128.97, 128.78, 128.72, |
| | 15.1 Hz, 1H), 3.79 (d, J = 15.1 Hz, 1H), | 128.64, 128.06, 127.75, 126.43, |
| | 3.63 (dd, J = 11.1, 3.6 Hz, 1H), 3.48 (dd, J = | 66.79, 48.32, 35.68, 16.75 |
| | 11.1, 5.8 Hz, 1H), 1.79-1.46 (m, 1H), 1.12 | |
| | (d, J = 6.9 Hz, 3H) | |
| A-27 | 1H NMR (400 MHz, CDCl3) δ 7.65-7.61 | 13C NMR (101 MHz, CDCl3) δ |
| | (m, 2H), 7.57-7.53 (m, 2H), 7.39-7.30 (m, | 169.90, 158.97, 147.41, 136.44, |
| | 6H), 4.44 (s, 2H), 2.99 (s, 3H), 2.87 (tt, J = | 132.31, 128.79, 128.78, 128.65, |
| | 6.8, 3.7 Hz, 1H), 1.02-0.94 (m, 2H), 0.91- | 128.32, 127.97, 126.53, 36.76, |
| | 0.82 (m, 2H) | 34.72, 31.58, 9.32 |
| A-28 | 1H NMR (400 MHz, CDCl3) δ 7.66-7.61 | 13C NMR (101 MHz, CDCl3) δ |
| | (m, 4H), 7.45-7.37 (m, 6H), 6.65 (q, J = | 163.49, 153.66, 150.26, 137.49, |
| | 6.0, 5.4 Hz, 1H), 4.24 (q, J = 7.2 Hz, 1H), | 130.48, 130.44, 129.35, 129.11, |
| | 2.90 (d, J = 4.9 Hz, 3H), 1.74 (d, J = 7.2 Hz, | 128.92, 128.27, 127.53, 127.00, |
| | 3H) | 65.57, 27.33, 11.71 |
| A-29 | 1H NMR (400 MHz, CDCl3) δ 7.67-7.60 | 13C NMR (101 MHz, CDCl3) δ |
| | (m, 2H), 7.57-7.52 (m, 2H), 7.40-7.28 (m, | 166.04, 158.94, 147.25, 136.30, |
| | 6H), 4.28 (s, 2H), 3.49-3.40 (m, 4H), 1.26 | 132.16, 128.65, 128.61, 128.57, |
| | (t, J = 7.1 Hz, 3H), 1.15 (t, J = 7.1 Hz, 3H) | 128.53, 128.26, 127.92, 126.37, |
| | | 42.63, 40.83, 36.51, 14.41, 12.94 |
| A-30 | 1H NMR (600 MHz, CDCl3) δ 7.64-7.60 | 13C NMR (151 MHz, CDCl3) δ |
| | (m, 2H), 7.58-7.55 (m, 2H), 7.44 (d, J = 6.4 | 170.50, 159.27, 147.55, 135.89, |
| | Hz, 1H), 7.41-7.32 (m, 6H), 4.25 (q, J = 7.4 | 131.67, 128.85, 128.75, 128.66, |
| | Hz, 1H), 4.18 (qt, J = 7.2, 5.4 Hz, 1H), 1.93- | 128.52, 128.20, 127.68, 126.41, |
| | 1.81 (m, 2H), 1.62 (d, J = 7.3 Hz, 3H), 1.53- | 51.42, 43.89, 32.97, 32.83, 23.65, |
| | 1.43 (m, 4H), 1.41-1.34 (m, 1H), 1.34- | 23.64, 16.67 |
| | 1.27 (m, 1H) | |
| A-31 | 1H NMR (400 MHz, CDCl3) δ 7.65-7.60 | 13C NMR (101 MHz, CDCl3) δ |
| | (m, 2H), 7.59-7.54 (m, 2H), 7.39-7.27 (m, | 208.50, 162.45, 145.32, 135.12, |
| | 6H), 3.22-3.09 (m, 4H), 2.01 (tt, J = 7.9, 4.6 | 132.58, 129.05, 128.63, 128.56, |
| | Hz, 1H), 1.12-1.05 (m, 2H), 0.95-0.88 (m, | 128.39, 128.03, 127.94, 126.45, |
| | 2H | 39.66, 22.31, 20.64, 10.87 |
| A-32 | 1H NMR (400 MHz, CDCl3) δ 7.52-7.47 | 13C NMR (101 MHz, CDCl3) δ |
| | (m, 2H), 7.47-7.41 (m, 2H), 7.19-7.15 (m, | 172.01, 162.07, 145.35, 138.41, |
| | 2H), 7.15-7.12 (m, 2H), 6.28-6.05 (m, | 137.81, 134.40, 129.63, 129.32, |
| | 1H), 3.18 (t, J = 7.3 Hz, 2H), 2.81 (d, J = 4.8 | 129.26, 127.71, 126.39, 126.18, |
| | Hz, 3H), 2.74 (t, J = 7.3 Hz, 2H), 2.37 (s, | 33.02, 26.40, 24.09, 21.36, 21.33 |
| | 3H), 2.36 (s, 3H) | |
| A-33 | 1H NMR (400 MHz, CDCl3) δ 7.65-7.61 | 13C NMR (101 MHz, CDCl3) δ |
| | (m, 2H), 7.59-7.54 (m, 2H), 7.41-7.30 (m, | 170.26, 159.41, 147.53, 135.92, |
| | 6H), 7.21 (s, 1H), 4.21 (q, J = 7.3 Hz, 1H), | 131.68, 128.82, 128.74, 128.63, |
| | 1.60 (d, J = 7.3 Hz, 3H), 1.27 (s, 9H) | 128.48, 128.25, 127.73, 126.43, |
| | | 51.28, 44.60, 28.53, 16.68 |
| A-34 | 1H NMR (600 MHz, CDCl3) δ 7.65-7.61 | 13C NMR (151 MHz, CDCl3) δ |
| | (m, 2H), 7.59-7.55 (m, 2H), 7.40-7.28 (m, | 171.05, 162.79, 145.27, 135.09, |
| | 6H), 3.24-3.20 (m, 2H), 3.07 (s, 3H), 2.99 | 132.62, 129.05, 128.61, 128.56, |
| | (s, 3H), 2.94-2.89 (m, 2H) | 128.36, 128.02, 127.98, 126.44, |
| | | 37.16, 35.57, 30.16, 23.79 |
| A-35 | 1H NMR (400 MHz, CDCl3) δ 7.68-7.63 | 13C NMR (101 MHz, CDCl3) δ |
| | (m, 2H), 7.63-7.59 (m, 2H), 7.48 (d, J = | 165.18, 158.30, 146.73, 137.60, |
| | 15.5 Hz, 1H), 7.42-7.31 (m, 6H), 6.87 (d, J = | 132.01, 129.25, 128.91, 128.83, |
| | 15.5 Hz, 1H), 5.83 (q, J = 4.1 Hz, 1H), | 128.69, 128.45, 128.20, 127.50, |
| | 2.97 (d, J = 4.9 Hz, 3H) | 126.86, 125.41, 26.84 |
| A-36 | 1H NMR (400 MHz, CDCl3) δ 7.67-7.61 | 13C NMR (101 MHz, CDCl3) δ |
| | (m, 2H), 7.61-7.53 (m, 2H), 7.42-7.28 (m, | 172.82, 162.79, 145.38, 134.98, |
| | 6H), 6.04-5.71 (m, 1H), 2.92 (t, J = 7.1 Hz, | 132.50, 128.96, 128.67, 128.60, |
| | 2H), 2.81 (d, J = 4.8 Hz, 3H), 2.35 (t, J = 7.3 | 128.48, 128.10, 127.89, 126.46, |
| | Hz, 2H), 2.24-2.15 (m, 2H) | 35.37, 27.29, 26.30, 23.08 |
| A-37 | 1H NMR (400 MHz, CDCl3) δ 7.63-7.59 | 13C NMR (101 MHz, CDCl3) δ |
| | (m, 2H), 7.57-7.52 (m, 2H), 7.44 (t, J = 5.5 | 171.17, 159.21, 147.62, 135.98, |
| | Hz, 1H), 7.41-7.32 (m, 6H), 4.32 (q, J = 7.4 | 131.66, 128.84, 128.74, 128.65, |
| | Hz, 1H), 3.16-3.02 (m, 2H), 1.76-1.66 (m, | 128.50, 128.20, 127.77, 126.40, |
| | 1H), 1.64 (d, J = 7.4 Hz, 3H), 0.80 (d, J = | 47.16, 43.91, 28.38, 20.00, 19.97, |
| | 6.7 Hz, 3H), 0.80 (d, J = 6.7 Hz, 3H) | 16.84 |
| A-38 | 1H NMR (600 MHz, CDCl3) δ 7.64-7.60 | 13C NMR (151 MHz, CDCl3) δ |
| | (m, 2H), 7.58-7.55 (m, 2H), 7.46 (d, J = 7.5 | 167.53, 159.25, 147.87, 135.99, |
| | Hz, 1H), 7.42-7.32 (m, 6H), 4.19 (qt, J = | 131.60, 128.89, 128.76, 128.67, |
| | 7.1, 5.5 Hz, 1H), 3.79 (s, 2H), 1.93-1.85 | 128.55, 128.16, 127.68, 126.40, |
| | (m, 2H), 1.53-1.47 (m, 4H), 1.40-1.33 (m, | 51.54, 35.46, 32.90, 23.60 |
| | 2H) | |
| A-39 | 1H NMR (400 MHz, CDCl3) δ 7.62-7.57 | 13C NMR (101 MHz, CDCl3) δ |
| | (m, 2H), 7.56-7.52 (m, 2H), 7.39-7.28 (m, | 171.03, 162.55, 145.20, 135.42, |
| | 6H), 5.77 (q, J = 4.8 Hz, 1H), 2.86 (d, J = | 132.53, 128.99, 128.80, 128.74, |
| | 4.8 Hz, 3H), 2.69 (ddd, J = 9.1, 6.0, 4.0 Hz, | |
| | 1H), 2.09 (ddd, J = 8.5, 5.6, 4.0 Hz, 1H), | 128.56, 128.29, 128.08, 126.46, |
| | 1.70 (ddd, J = 9.4, 5.6, 4.2 Hz, 1H), 1.57 | 26.83, 24.94, 17.57, 15.05 |
| | (ddd, J = 8.5, 6.0, 4.2 Hz, 1H) | |
| A-40 | 1H NMR (400 MHz, CDCl3) δ 7.64-7.59 | 13C NMR (101 MHz, CDCl3) δ |
| | (m, 2H), 7.59-7.53 (m, 2H), 7.39-7.28 (m, | 209.24, 162.42, 145.34, 135.09, |
| | 6H), 3.17-3.09 (m, 2H), 3.05-2.99 (m, | 132.55, 129.04, 128.64, 128.56, |
| | 2H), 2.54 (q, J = 7.3 Hz, 2H), 1.11 (t, J = 7.3 | 128.41, 128.03, 127.92, 126.46, |
| | Hz, 3H) | 38.61, 36.07, 22.23, 7.84 |
| A-42 | 1H NMR (400 MHz, CDCl3) δ 7.65-7.59 | 13C NMR (101 MHz, CDCl3) δ |
| | (m, 2H), 7.59-7.52 (m, 2H), 7.42-7.30 (m, | 167.28, 158.97, 148.11, 136.11, |
| | 6H), 7.19 (d, J = 8.5 Hz, 1H), 3.83 (s, 2H), | 131.62, 128.89, 128.76, 128.66, |
| | 3.45 (qd, J = 9.1, 4.1 Hz, 1H), 1.99-1.88 | 128.53, 127.69, 126.44, 54.56, |
| | (m, 1H), 1.73-1.55 (m, 3H), 1.36-1.23 (m, | 38.34, 35.62, 34.13, 33.32, 25.62, |
| | 1H), 1.23-1.13 (m, 1H), 1.14-1.08 (m, | 25.26, 18.90 |
| | 1H), 1.09-0.97 (m, 2H), 0.84 (d, J = 6.4 Hz, | |
| | 3H) | |
| A-43 | 1H NMR (400 MHz, DMSO) δ 8.57 (d, J = | 13C NMR (101 MHz, DMSO) δ |
| | 7.7 Hz, 1H), 7.60-7.54 (m, 2H), 7.54-7.49 | 165.13, 158.18, 146.50, 135.66, |
| | (m, 2H), 7.47-7.36 (m, 6H), 4.27-4.13 (m, | 131.32, 128.89, 128.87, 128.60, |
| | 1H), 4.01 (s, 2H), 2.21-2.09 (m, 2H), 1.95- | 128.40, 127.77, 127.30, 126.08, |
| | 1.82 (m, 2H), 1.69-1.53 (m, 2H) | 44.26, 35.52, 30.04, 14.58 |
| A-44 | 1H NMR (400 MHz, DMSO) δ 10.10-9.75 | 13C NMR (101 MHz, DMSO) δ |
| | (m, 2H), 7.63 (d, J = 8.6 Hz, 4H), 7.61 (d, J = | 158.37, 145.79, 134.61, 131.28, |
| | 7.8 Hz, 4H), 7.46 (dq, J = 12.4, 7.2 Hz, | 129.41, 129.05, 128.84, 128.64, |
| | 12H), 4.74 (dd, J = 11.3, 3.5 Hz, 1H), 3.77- | 127.68, 127.49, 126.58, 56.62, |
| | 3.63 (m, 1H), 3.38-3.30 (m, 1H), 3.18 (d, J = | 51.52, 43.87, 43.09, 26.81, 26.74, |
| | 12.4 Hz, 1H), 3.10 (q, J = 10.3 Hz, 1H), | 21.58, 21.25, 20.90 |
| | 2.87 (td, J = 12.5, 3.6 Hz, 1H), 2.30 (dd, J = | |
| | 14.1, 3.9 Hz, 1H), 2.13 (d, J = 12.4 Hz, OH), | |
| | 2.07-1.93 (m, 2H), 1.90-1.42 (m, 9H) | |
| A-45 | 1H NMR (400 MHz, DMSO) δ 8.15 (d, J = | 13C NMR (101 MHz, DMSO) δ |
| | 0.9 Hz, 1H), 7.90-7.84 (m, 1H), 7.26 (d, J = | 170.02, 169.93, 159.10, 141.37, |
| | 0.9 Hz, 1H), 3.33 (t, J = 6.9 Hz, 2H), 2.57 (d, | 128.25, 34.89, 34.84, 27.78, 25.36, |
| | J = 4.5 Hz, 3H), 2.55 (t, J = 6.9 Hz, 2H) | 25.23 |
| A-46 | 1H NMR (400 MHz, CDCl3) δ 7.64-7.60 | 13C NMR (101 MHz, CDCl3) δ |
| | (m, 2H), 7.58-7.53 (m, 2H), 7.41-7.31 (m, | 167.36, 159.12, 147.92, 136.10, |
| | 6H), 7.26 (d, J = 8.2 Hz, 1H), 3.85 (dp, J = | 131.60, 128.88, 128.76, 128.64, |
| | 9.0, 6.8 Hz, 1H), 3.83 (s, 2H), 1.70-1.57 | 128.53, 128.18, 127.76, 126.41, |
| | (m, J = 6.7, 5.7 Hz, 1H), 1.02 (d, J = 6.8 Hz, | |
| | 3H), 0.78 (d, J = 6.8 Hz, 3H), 0.77 (d, J = | 50.49, 35.59, 32.86, 18.39, 18.13, |
| | 6.8 Hz, 3H) | 17.36 |
| A-47 | 1H NMR (400 MHz, DMSO) δ 12.68 (s, 1H), | 13C NMR (101 MHz, DMSO) δ |
| | 8.21 (q, J = 4.7 Hz, 1H), 7.55-7.14 (m, | 171.20, 171.11, 138.58, 128.56, |
| | 10H), 4.15 (q, J = 7.0 Hz, 1H), 2.63-2.62 | 128.13, 127.64, 126.93, 126.55, |
| | (m, 3H), 1.47 (d, J = 7.0 Hz, 3H) | 45.97, 45.93, 25.73, 25.59, 19.09 |
| A-48 | 1H NMR (400 MHz, DMSO) δ 7.58-7.54 | 13C NMR (101 MHz, DMSO) δ |
| | (m, 2H), 7.53-7.49 (m, 2H), 7.45-7.41 (m, | 207.50, 156.91, 147.07, 135.81, |
| | 6H), 4.62 (q, J = 7.1 Hz, 1H), 2.84 (dq, J = | 131.25, 129.17, 129.04, 128.76, |
| | 18.2, 7.2 Hz, 1H), 2.75 (dq, J = 18.3, 7.2 Hz, | 128.55, 127.73, 127.34, 126.30, |
| | 1H), 1.55 (d, J = 7.1 Hz, 3H), 0.99 (t, J = 7.2 | 50.01, 32.51, 17.00, 7.82 |
| | Hz, 3H) | |
| A-49 | 1H NMR (400 MHz, DMSO) δ 7.91 (q, J = | 13C NMR (101 MHz, DMSO) δ |
| | 4.3 Hz, 2H), 7.61-7.55 (m, 2H), 7.55-7.50 | 170.05, 169.96, 158.65, 157.08, |
| | (m, 2H), 7.48-7.35 (m, 6H), 3.43 (t, J = 6.8 | 146.48, 135.65, 131.37, 128.90, |
| | Hz, 2H), 2.66 (t, J = 6.8 Hz, 2H), 2.59-2.58 | 128.86, 128.63, 128.38, 127.79, |
| | (m, 3H) | 127.36, 126.12, 34.87, 34.83, |
| | | 27.81, 25.37, 25.24 |
| A-50 | 1H NMR (400 MHz, CDCl3) δ 7.68 (d, J = | 13C NMR (101 MHz, CDCl3) δ |
| | 0.9 Hz, 2H), 7.33-7.19 (m, 2H), 7.12 (d, J = | 171.75, 171.66, 160.28, 140.53, |
| | 0.9 Hz, 2H), 4.20 (q, J = 7.3 Hz, 2H), 2.82 | 128.03, 43.96, 43.90, 26.53, 26.38, |
| | (s, 3H), 2.80 (s, 3H), 1.59 (d, J = 7.4 Hz, 6H) | 17.05 |
| A-51 | 1H NMR (400 MHz, DMSO) δ 7.56-7.52 | 13C NMR (101 MHz, DMSO) δ |
| | (m, 2H), 7.52-7.49 (m, 2H), 7.47-7.34 (m, | 206.91, 158.16, 146.52, 135.58, |
| | 6H), 4.51 (s, 2H), 2.91 (hept, J = 6.9 Hz, | 131.25, 128.91, 128.90, 128.62, |
| | 1H), 1.10 (d, J = 6.9 Hz, 6H) | 128.38, 127.74, 127.18, 126.15, |
| | | 40.42, 39.01, 17.97 |
| A-52 | 1H NMR (400 MHz, CDCl3) δ 7.65-7.58 | 13C NMR (101 MHz, CDCl3) δ |
| | (m, 2H), 7.58-7.51 (m, 2H), 7.42-7.30 (m, | 167.26, 159.01, 147.90, 136.06, |
| | 6H), 7.20 (d, J = 8.5 Hz, 1H), 4.01-3.89 (m, | 131.61, 128.90, 128.77, 128.67, |
| | 1H), 3.81 (s, 2H), 1.38-1.30 (m, 2H), 1.29- | 128.53, 128.20, 127.69, 126.44, |
| | 1.18 (m, 2H), 1.08 (d, J = 6.6 Hz, 3H), 0.78 | 45.62, 38.93, 35.57, 20.80, 19.07, |
| | (t, J = 7.2 Hz, 3H) | 13.87 |
| A-53 | 1H NMR (400 MHz, CDCl3) δ 7.68-7.64 | 13C NMR (101 MHz, CDCl3) δ |
| | (m, 2H), 7.57-7.53 (m, 2H), 7.41-7.30 (m, | 168.25, 159.03, 147.84, 136.14, |
| | 6H), 3.86 (s, 2H), 3.50-3.45 (m, 2H), 3.43- | 131.67, 128.84, 128.75, 128.64, |
| | 3.38 (m, 2H), 3.15 (s, 3H) | 128.45, 128.30, 127.80, 126.45, |
| | | 70.93, 58.62, 39.73, 35.25 |
| A-54 | 1H NMR (400 MHz, CDCl3) δ 7.69-7.61 | 13C NMR (101 MHz, CDCl3) δ |
| | (m, 2H), 7.56-7.51 (m, 2H), 7.48-7.43 (m, | 158.69, 147.22, 136.54, 136.47, |
| | 2H), 7.40-7.26 (m, 9H), 4.48 (s, 2H) | 132.17, 129.14, 128.70, 128.69, |
| | | 128.66, 128.60, 128.52, 128.26, |
| | | 127.92, 127.79, 126.39, 36.95 |
| A-55 | 1H NMR (400 MHz, DMSO) δ 7.60-7.55 | 13C NMR (101 MHz, DMSO) δ |
| | (m, 2H), 7.54-7.50 (m, 2H), 7.47-7.36 (m, | 158.67, 146.40, 135.68, 131.41, |
| | 6H), 3.28 (q, J = 7.3 Hz, 2H), 1.43 (t, J = 7.3 | 128.90, 128.85, 128.62, 128.36, |
| | Hz, 3H) | 127.82, 127.35, 126.11, 26.28, |
| | | 14.93 |
| A-56 | 1H NMR (400 MHz, DMSO) δ 11.84 (s, 1H), | 13C NMR (101 MHz, DMSO) δ |
| | 8.26 (d, J = 4.2 Hz, 1H), 3.74 (q, J = 7.0 Hz, | 171.99, 133.60, 133.24, 123.45, |
| | 1H), 2.64-2.55 (m, 1H), 2.05 (s, 3H), 1.99 | 46.05, 22.28, 18.42, 12.07, 9.00, |
| | (s, 3H), 1.29 (d, J = 7.0 Hz, 3H), 0.64-0.54 | 5.54, 5.46 |
| | (m, 2H), 0.38-0.30 (m, 2H) | |
| A-57 | 1H NMR (400 MHz, DMSO) δ 7.60-7.55 | 13C NMR (101 MHz, DMSO) δ |
| | (m, 2H), 7.55-7.51 (m, 2H), 7.48-7.36 (m, | 170.02, 158.02, 146.67, 135.68, |
| | 6H), 4.39 (t, J = 6.1 Hz, 2H), 3.54 (t, J = 6.1 | 131.27, 128.95, 128.92, 128.65, |
| | Hz, 2H), 1.97 (s, 3H) | 128.42, 127.72, 127.31, 126.16, |
| | | 62.18, 30.72, 20.43 |
| A-58 | 1H NMR (400 MHz, DMSO) δ 8.36 (d, J = | 13C NMR (101 MHz, DMSO) δ |
| | 4.2 Hz, 1H), 7.50-7.46 (m, 2H), 7.46-7.42 | 167.42, 159.46, 159.05, 157.08, |
| | (m, 2H), 7.03-6.98 (m, 2H), 6.99-6.95 (m, | 145.85, 134.28, 128.42, 127.76, |
| | 2H), 3.95 (s, 2H), 3.79 (s, 3H), 3.78 (s, 3H), | 123.72, 120.35, 114.34, 114.00, |
| | 2.69-2.61 (m, 1H), 0.66-0.60 (m, 2H), | 55.17, 55.06, 39.84, 35.44, 22.54, |
| | 0.42-0.37 (m, 2H) | 5.52 |
| A-59 | 1H NMR (400 MHz, DMSO) δ 8.18-8.12 | 13C NMR (101 MHz, DMSO) δ |
| | (m, 2H), 8.15 (d, J = 0.9 Hz, 2H), 7.24 (d, J = | 166.66, 166.57, 158.63, 141.43, |
| | 0.9 Hz, 2H), 3.92 (s, 4H), 2.61 (s, 3H), | 128.27, 35.39, 35.34, 25.84, 25.70. |
| | 2.60 (s, 3H) | |
| A-60 | 1H NMR (400 MHz, DMSO) δ 7.59-7.55 | 13C NMR (101 MHz, DMSO) δ |
| | (m, 2H), 7.55-7.50 (m, 2H), 7.47-7.36 (m, | 158.96, 146.49, 135.74, 131.49, |
| | 6H), 5.14 (t, J = 5.5 Hz, 1H), 3.76 (q, J = 6.1 | 129.00, 128.95, 128.72, 128.47, |
| | Hz, 2H), 3.36 (t, J = 6.3 Hz, 2H) | 127.91, 127.46, 126.20, 59.73, |
| | | 34.75 |
| A-61 | 1H NMR (400 MHz, DMSO) δ 7.57-7.53 | 13C NMR (101 MHz, DMSO) δ |
| | (m, 2H), 7.53-7.49 (m, 2H), 7.47-7.35 (m, | 204.19, 158.16, 146.55, 135.60, |
| | | 131.25, 128.91, 128.63, 128.40, |
| | 6H), 4.40 (s, 2H), 2.68 (q, J = 7.3 Hz, 2H), | 127.73, 127.23, 126.12, 41.37, |
| | 1.00 (t, J = 7.3 Hz, 3H) | 34.08, 7.57 |
| A-62 | 1H NMR (400 MHz, CDCl3) δ 10.10-9.94 | 13C NMR (101 MHz, CDCl3) δ |
| | (m, 1H), 7.62-7.57 (m, 2H), 7.56-7.51 (m, | 159.02, 146.35, 134.86, 131.68, |
| | 2H), 7.41-7.31 (m, 6H), 3.58-3.45 (m, | 128.91, 128.75, 128.70, 128.47, |
| | 4H), 2.82 (t, J = 5.4 Hz, 3H) | 128.21, 127.99, 126.60, 46.30, |
| | | 33.26, 24.45 |
| A-63 | 1H NMR (400 MHz, DMSO) δ 7.61-7.56 | 13C NMR (101 MHz, DMSO) δ |
| | (m, 2H), 7.56-7.52 (m, 2H), 7.48-7.32 (m, | 161.16, 156.60, 144.73, 134.25, |
| | 6H), 3.52 (s, 3H), 3.44 (q, J = 6.6 Hz, 2H), | 131.93, 128.83, 128.73, 128.55, |
| | 2.98 (t, J = 6.9 Hz, 2H) | 128.40, 128.04, 127.24, 126.33, |
| | | 69.68, 51.22, 37.93, 28.16 |
| A-64 | 1H NMR (400 MHz, DMSO) δ 12.44 (s, 2H), | 13C NMR (101 MHz, DMSO) δ |
| | 8.19 (s, 2H), 7.71 (d, J = 7.6 Hz, 4H), 7.65- | 168.10, 168.01, 139.77, 134.40, |
| | 7.56 (m, 2H), 7.35 (t, J = 7.7 Hz, 4H), 7.23- | 133.76, 132.41, 128.44, 126.23, |
| | 7.16 (m, 2H), 3.76 (s, 4H), 2.62 (s, 3H), 2.61 | 123.96, 36.52, 36.48, 25.79, 25.66 |
| | (s, 3H) | |
| A-65 | 1H NMR (400 MHz, CDCl3) δ 7.63-7.58 | 13C NMR (101 MHz, CDCl3) δ |
| | (m, 2H), 7.57-7.52 (m, 2H), 7.50-7.43 (m, | 168.11, 159.14, 147.94, 136.10, |
| | 1H), 7.42-7.30 (m, 6H), 3.85 (s, 2H), 3.10 | 131.59, 128.89, 128.77, 128.67, |
| | (t, J = 6.4 Hz, 2H), 1.81-1.65 (m, J = 6.7 | 128.54, 128.17, 127.77, 126.40, |
| | Hz, 1H), 0.81 (d, J = 6.7 Hz, 6H) | 47.24, 35.39, 28.34, 19.98 |
| A-66 | 1H NMR (400 MHz, DMSO) δ 8.29 (d, J = | 13C NMR (101 MHz, DMSO) δ |
| | 3.9 Hz, 1H), 8.15 (d, J = 0.9 Hz, 1H), 7.24 | 167.32, 167.24, 158.61, 141.43, |
| | (d, J = 0.9 Hz, 1H), 3.88 (s, 2H), 2.65-2.57 | 128.27, 35.47, 35.43, 22.50, 22.37, |
| | (m, 1H), 0.65-0.59 (m, 2H), 0.43-0.36 (m, | 5.53, 5.50 |
| | 2H) | |
| A-67 | 1H NMR (400 MHz, CDCl3) δ 7.68-7.64 | 13C NMR (101 MHz, CDCl3) δ |
| | (m, 2H), 7.59-7.54 (m, 2H), 7.45 (d, J = 8.1 | 167.54, 158.97, 147.80, 136.11, |
| | Hz, 1H), 7.42-7.31 (m, 6H), 4.19-4.10 (m, | 131.68, 128.83, 128.75, 128.64, |
| | 1H), 3.88-3.79 (m, 2H), 3.32-3.27 (m, | 128.46, 128.28, 127.77, 126.43, |
| | 2H), 3.18 (s, 3H), 1.15 (d, J = 6.8 Hz, 3H) | 75.29, 58.93, 45.42, 35.47, 17.41 |
| A-68 | 1H NMR (400 MHz, DMSO) δ 8.27 (q, J = | 13C NMR (101 MHz, DMSO) δ |
| | 5.0 Hz, 1H), 7.47-7.42 (m, 2H), 7.42-7.36 | 166.76, 157.64, 146.37, 138.46, |
| | (m, 2H), 7.28-7.23 (m, 2H), 7.24-7.19 (m, | 137.70, 135.15, 129.42, 129.15, |
| | 2H), 4.02 (s, 2H), 2.63 (d, J = 4.6 Hz, 3H), | 128.51, 127.13, 126.00, 125.06, |
| | 2.33 (s, 6H) | 35.39, 25.86, 20.79, 20.76 |
| A-69 | 1H NMR (400 MHz, DMSO) δ 8.28 (q, J = | 13C NMR (101 MHz, DMSO) δ |
| | 4.6 Hz, OH), 7.52-7.47 (m, 2H), 7.47-7.42 | 170.30, 170.21, 159.52, 159.07, |
| | (m, 2H), 7.05-7.00 (m, 2H), 7.00-6.95 (m, | 156.48, 146.04, 134.35, 128.49, |
| | 2H), 4.40 (q, J = 6.9 Hz, 1H), 3.79 (s, 3H), | 127.80, 123.69, 120.29, 114.37, |
| | 3.78 (s, 3H), 2.64-2.60 (m, 3H), 1.58 (d, J = | 114.03, 55.17, 55.07, 45.98, 45.93, |
| | 7.0 Hz, 3H) | 25.77, 25.64, 19.42 |
| A-70 | 1H NMR (400 MHz, CDCl3) δ 7.64-7.59 | 13C NMR (101 MHz, CDCl3) δ |
| | (m, 2H), 7.58-7.53 (m, 2H), 7.42-7.31 (m, | 167.98, 159.06, 147.92, 136.07, |
| | 6H), 3.83 (s, 2H), 3.27 (q, J = 5.7 Hz, 2H), | 131.60, 128.89, 128.76, 128.67, |
| | 1.47-1.39 (m, 2H), 1.29-1.18 (m, 2H), | 128.53, 128.18, 127.71, 126.42, |
| | 0.79 (t, J = 7.3 Hz, 3H) | 39.63, 35.35, 31.30, 19.98, 13.62 |
| A-71 | 1H NMR (400 MHz, DMSO) δ 7.58-7.53 | 13C NMR (101 MHz, DMSO) δ |
| | (m, 2H), 7.47-7.42 (m, 2H), 7.42-7.31 (m, | 160.42, 138.41, 135.16, 132.87, |
| | 5H), 7.29-7.23 (m, 1H), 3.07 (s, 6H) | 129.05, 128.64, 128.38, 127.87, |
| | | 127.43, 127.12, 124.82, 37.25 |
| A-72 | 1H NMR (400 MHz, DMSO) δ 8.22 (d, J = | 13C NMR (101 MHz, DMSO) δ |
| | 5.2 Hz, 1H), 7.50-7.46 (m, 2H), 7.45-7.41 | 166.78, 166.70, 159.46, 159.05, |
| | (m, 2H), 7.03-6.99 (m, 2H), 6.99-6.95 (m, | 157.10, 145.86, 134.29, 128.45, |
| | 2H), 4.00 (s, 2H), 3.79 (s, 3H), 3.78 (s, 3H), | 127.73, 123.71, 120.35, 114.36, |
| | 2.65-2.62 (m, 3H) | 114.02, 55.17, 55.06, 35.40, 35.36, |
| | | 25.87, 25.74 |
| A-73 | 1H NMR (400 MHz, CDCl3) δ 7.65-7.60 | 13C NMR (101 MHz, CDCl3) δ |
| | (m, 2H), 7.59-7.54 (m, 2H), 7.39-7.28 (m, | 172.49, 161.72, 145.45, 135.16, |
| | 6H), 3.73 (s, 3H), 3.22-3.15 (m, 2H), 2.95- | 132.48, 129.00, 128.65, 128.56, |
| | 2.89 (m, 2H) | 128.46, 128.07, 127.93, 126.48, |
| | | 51.95, 30.93, 23.55 |
| B-01 | 1H NMR (600 MHz, DMSO) δ 8.38 (d, J = | 13C NMR (151 MHz, DMSO) δ |
| | 4.3 Hz, 1H), 7.52-7.36 (m, 10H), 4.57 (q, J = | 171.98, 170.00, 156.06, 154.59, |
| | 7.0 Hz, 1H), 2.64 (ddt, J = 11.4, 7.3, 4.0 | 135.69, 135.41, 131.31, 130.20, |
| | Hz, 1H), 1.58 (d, J = 7.0 Hz, 3H), 0.64-0.57 | 129.80, 129.74, 128.95, 128.86, |
| | (m, 2H), 0.41-0.36 (m, 1H), 0.34-0.29 (m, | 44.28, 23.06, 19.24, 6.11, 6.07 |
| | 1H) | |
| B-02 | 1H NMR (600 MHz, CDCl3) δ 8.50 (s, 1H), | 13C NMR (151 MHz, CDCl3) δ |
| | 7.44-7.40 (m, 4H), 7.38-7.35 (m, 1H), | 172.49, 152.41, 151.35, 148.49, |
| | 7.35-7.27 (m, 5H), 7.08-7.05 (m, 1H), | 140.90, 138.12, 137.87, 129.55, |
| | 4.40 (q, J = 7.4 Hz, 1H), 2.68 (d, J = 4.9 Hz, | 129.38, 129.07, 128.63, 128.48, |
| | 3H), 1.64 (d, J = 7.4 Hz, 3H) | 128.35, 41.64, 26.38, 17.03 |
| B-03 | 1H NMR (600 MHz, CDCl3) δ 7.55-7.52 | 13C NMR (151 MHz, CDCl3) δ |
| | (m, 2H), 7.52-7.49 (m, 2H), 7.46-7.41 (m, | 171.69, 170.29, 156.01, 154.31, |
| | 2H), 7.38 (dd, J = 8.2, 6.7 Hz, 2H), 7.33 (t, J = | 135.03, 134.74, 131.22, 129.85, |
| | 7.7 Hz, 2H), 6.89 (q, J = 5.0 Hz, 1H), 4.42 | 129.68, 129.25, 128.73, 128.60, |
| | (t, J = 7.4 Hz, 1H), 2.81 (d, J = 4.9 Hz, 3H), | 49.20, 26.57, 24.40, 12.12 |
| | 2.22 (dp, J = 14.6, 7.3 Hz, 1H), 1.96 (dp, J = | |
| | 14.7, 7.4 Hz, 1H), 1.13 (t, J = 7.4 Hz, 3H) | |
| B-04 | 1H NMR (600 MHz, CDCl3) δ 8.54 (s, 1H), | 13C NMR (151 MHz, CDCl3) δ |
| | 7.44-7.41 (m, 4H), 7.38-7.34 (m, 1H), | 169.11, 151.53, 151.52, 148.71, |
| | 7.34-7.28 (m, 5H), 6.99-6.96 (m, 1H), | 140.71, 137.94, 137.86, 129.55, |
| | 3.88 (s, 2H), 2.72 (d, J = 4.9 Hz, 3H) | 129.47, 129.11, 128.66, 128.44, |
| | | 128.37, 33.32, 26.48 |
| B-05 | 1H NMR (400 MHz, CDCl3) δ 7.55-7.47 | 13C NMR (101 MHz, CDCl3) δ |
| | (m, 4H), 7.46-7.27 (m, 6H), 5.17 (dd, J = | 170.98, 170.27, 155.83, 154.14, |
| | 7.7, 6.6 Hz, 1H), 3.22 (s, 3H), 3.03 (s, 3H), | 135.20, 135.06, 130.96, 129.80, |
| | 2.20 (dp, J = 13.8, 7.3 Hz, 1H), 2.03 (dp, J = | 129.48, 129.30, 128.64, 128.49, |
| | 14.0, 7.5 Hz, 1H), 1.11 (t, J = 7.4 Hz, 3H) | 46.07, 37.88, 36.23, 26.34, 11.86 |
| B-06 | 1H NMR (400 MHz, CDCl3) δ 7.55-7.51 | 13C NMR (101 MHz, CDCl3) δ |
| | (m, 2H), 7.51-7.46 (m, 2H), 7.46-7.30 (m, | 169.77, 167.23, 155.79, 154.23, |
| | 6H), 4.38 (t, J = 7.7 Hz, 2H), 4.10 (t, J = 7.8 | 135.20, 135.14, 130.91, 129.83, |
| | Hz, 2H), 3.96 (s, 2H), 2.36-2.25 (m, 2H) | 129.46, 129.35, 128.62, 128.47, |
| | | 50.83, 48.61, 31.03, 15.39 |
| B-07 | 1H NMR (400 MHz, CDCl3) δ 9.34 (dd, J = | 13C NMR (101 MHz, CDCl3) δ |
| | 7.9, 1.5 Hz, 1H), 9.14 (dd, J = 8.1, 1.5 Hz, | 172.30, 169.73, 143.76, 143.40, |
| | 1H), 8.60-8.53 (m, 2H), 7.90 (ddd, J = 8.4, | 134.28, 132.98, 130.71, 128.77, |
| | 7.1, 1.5 Hz, 1H), 7.83 (ddd, J = 8.2, 7.1, 1.6 | 128.51, 127.32, 127.08, 127.05, |
| | Hz, 1H), 7.81-7.73 (m, 2H), 6.96 (q, J = 4.9 | 124.50, 123.11, 123.08, 42.99, |
| | Hz, 1H), 4.68 (q, J = 7.4 Hz, 1H), 2.81 (d, J = | 26.71, 17.10 |
| | 4.9 Hz, 3H), 1.77 (d, J = 7.5 Hz, 3H) | |
| B-11 | 1H NMR (600 MHz, CDCl3) δ 7.55-7.53 | 13C NMR (151 MHz, CDCl3) δ |
| | (m, 2H), 7.52-7.49 (m, 2H), 7.46-7.41 (m, | 172.21, 170.15, 156.05, 154.35, |
| | 2H), 7.40-7.36 (m, 2H), 7.35-7.31 (m, | 134.99, 134.69, 131.26, 129.84, |
| | 2H), 7.01 (q, J =4.8 Hz, 1H), 4.59 (q, J = | 129.70, 129.25, 128.74, 128.60, |
| | 7.37 Hz, 1H), 2.31 (d, J = 4.85 Hz, 3H), 1.68 | 41.98, 26.61, 16.83 |
| | (d, J = 7.4 Hz, 3H) | |
| B-14 | 1H NMR (600 MHz, CDCl3) δ 7.47-7.43 | 13C NMR (151 MHz, CDCl3) δ |
| | (m, 2H), 7.42-7.38 (m, 2H), 7.20-7.16 (m, | 172.31, 169.63, 155.85, 154.20, |
| | 2H), 7.15-7.10 (m, 3H), 4.57 (q, J = 7.4 Hz, | 141.85, 139.78, 132.27, 131.91, |
| | 1H), 2.79 (d, J = 4.9 Hz, 3H), 2.38 (s, 3H), | 129.80, 129.42, 129.29, 129.06, |
| | 2.36 (s, 3H), 1.66 (d, J = 7.4 Hz, 3H) | 41.94, 26.57, 21.51, 21.39, 16.86 |
| B-15 | 1H NMR (400 MHz, DMSO) δ 7.50-7.44 | 13C NMR (101 MHz, DMSO) δ |
| | (m, 5H), 7.45-7.36 (m, 5H), 5.13 (q, J = 6.9 | 170.28, 169.37, 155.78, 154.20, |
| | Hz, 1H), 3.13 (s, 3H), 2.88 (s, 3H), 1.58 (d, J = | 135.17, 134.99, 130.82, 129.63, |
| | 6.9 Hz, 3H) | 129.33, 129.27, 128.46, 128.40, |
| | | 40.58, 37.18, 35.59, 18.10 |
| B-17 | 1H NMR (400 MHz, DMSO) δ 8.18 (q, J = | 13C NMR (101 MHz, DMSO) δ |
| | 5.0 Hz, 1H), 7.52-7.43 (m, 5H), 7.46-7.35 | 169.61, 167.13, 155.45, 154.06, |
| | (m, 5H), 4.04 (s, 2H), 2.63 (d, J = 4.6 Hz, | 135.20, 134.90, 130.86, 129.71, |
| | 3H) | 129.34, 129.24, 128.49, 128.38, |
| | | 34.09, 25.99 |
| B-18 | 1H NMR (400 MHz, CDCl3) δ 7.53-7.47 | 13C NMR (101 MHz, CDCl3) δ |
| | (m, 4H), 7.45-7.38 (m, 2H), 7.38-7.29 (m, | 170.18, 169.64, 155.85, 154.12, |
| | 4H), 5.02 (q, J = 7.0 Hz, 1H), 3.81 (dt, J = | 135.21, 135.14, 130.90, 129.77, |
| | 10.0, 6.8 Hz, 1H), 3.58-3.48 (m, 3H), 2.02- | 129.46, 129.32, 128.62, 128.49, |
| | 1.92 (m, 2H), 1.91-1.82 (m, 2H), 1.72 (d, J = | 46.74, 46.40, 42.15, 26.16, 24.31, |
| | 7.0 Hz, 3H) | 18.03 |
| B-19 | 1H NMR (600 MHz, DMSO) δ 8.33 (d, J = | 13C NMR (151 MHz, DMSO) δ |
| | 4.1 Hz, 1H), 7.52-7.35 (m, 10H), 4.00 (s, | 170.21, 168.23, 155.85, 154.49, |
| | 2H), 2.66-2.60 (m, 1H), 0.61-0.57 (m, | 135.70, 135.38, 131.34, 130.23, |
| | 2H), 0.38-0.34 (m, 2H) | 129.80, 129.73, 128.97, 128.83, |
| | | 34.62, 23.16, 6.04 |
| B-21 | 1H NMR (400 MHz, CDCl3) δ 7.50-7.44 | 13C NMR (101 MHz, CDCl3) δ |
| | (m, 2H), 7.44-7.39 (m, 2H), 7.21-7.16 (m, | 171.25, 169.60, 155.62, 154.10, |
| | 2H), 7.17-7.12 (m, 2H), 7.12-7.09 (m, | 141.76, 139.71, 132.39, 132.10, |
| | 1H), 4.42 (t, J = 7.3 Hz, 1H), 3.47-3.39 (m, | 129.82, 129.40, 129.33, 129.11, |
| | 2H), 3.39-3.32 (m, 2H), 3.17 (s, 3H), 2.39 | 70.98, 58.65, 49.31, 39.48, 24.40, |
| | (s, 3H), 2.37 (s, 3H), 2.20 (dp, J = 14.6, 7.3 | 21.52, 21.40, 12.07 |
| | Hz, 1H), 1.96 (dp, J = 14.7, 7.4 Hz, 1H), | |
| | 1.13 (t, J = 7.4 Hz, 3H) | |
| B-22 | 1H NMR (400 MHz, CDCl3) δ 7.58-7.54 | 13C NMR (101 MHz, CDCl3) δ |
| | (m, 2H), 7.53-7.50 (m, 2H), 7.48-7.32 (m, | 170.90, 170.18, 155.73, 154.27, |
| | 6H), 6.92 (d, J = 7.5 Hz, 1H), 4.51 (q, J = | 134.96, 134.78, 131.27, 129.83, |
| | 7.3 Hz, 1H), 4.16 (h, J = 7.3 Hz, 1H), 1.93- | 129.69, 129.30, 128.73, 128.66, |
| | 1.78 (m, 2H), 1.67 (d, J = 7.4 Hz, 3H), 1.53- | 51.38, 42.19, 32.94, 32.77, 23.62, |
| | 1.41 (m, 4H), 1.32-1.15 (m, 2H) | 23.59, 16.71 |
| B-23 | 1H NMR (600 MHz, CDCl3) δ 8.53 (s, 1H), | 13C NMR (151 MHz, CDCl3) δ |
| | 7.42-7.38 (m, 4H), 7.34-7.31 (m, 1H), | 167.91, 152.19, 151.23, 147.96, |
| | | 140.55, 138.49, 138.22, 129.72, |
| | 7.30-7.26 (m, 5H), 4.16 (s, 2H), 3.09 (s, | 129.57, 128.69, 128.35, 128.26, |
| | 3H), 3.00 (s, 3H) | 128.08, 37.77, 36.11, 32.34 |
| B-24 | 1H NMR (400 MHz, CDCl3) δ 8.54 (s, 1H), | 13C NMR (101 MHz, CDCl3) δ |
| | 7.47-7.41 (m, 4H), 7.40-7.27 (m, 6H), | 170.15, 151.89, 151.38, 148.63, |
| | 3.81 (s, 2H), 2.62 (dh, J = 7.2, 3.6 Hz, 1H), | 140.81, 137.98, 137.70, 129.57, |
| | 0.58-0.51 (m, 2H), 0.06-0.00 (m, 2H) | 129.43, 129.20, 128.71, 128.55, |
| | | 128.38, 33.28, 22.64, 6.07 |
| B-25 | 1H NMR (400 MHz, DMSO) δ 7.49-7.34 | 13C NMR (101 MHz, DMSO) δ |
| | (m, 10H), 4.46 (s, 2H), 2.91 (hept, J = 6.9 | 206.88, 169.19, 155.48, 154.00, |
| | Hz, 1H), 1.06 (d, J = 6.9 Hz, 6H) | 135.01, 134.80, 130.69, 129.53, |
| | | 129.20, 129.18, 128.34, 128.22, |
| | | 39.20, 38.81, 17.99 |
| B-26 | 1H NMR (600 MHz, CDCl3) δ 7.54-7.49 | 13C NMR (151 MHz, CDCl3) δ |
| | (m, 2H), 7.49-7.45 (m, 2H), 7.43-7.36 (m, | 171.50, 170.44, 155.69, 153.90, |
| | 2H), 7.36-7.27 (m, 4H), 6.21 (q, J = 5.6 Hz, | 135.10, 134.94, 131.00, 129.78, |
| | 1H), 3.59 (t, J = 7.1 Hz, 2H), 2.78 (d, J = 4.9 | 129.49, 129.23, 128.61, 128.50, |
| | Hz, 3H), 2.76 (t, J = 7.0 Hz, 2H) | 36.04, 26.75, 26.32 |
| B-27 | 1H NMR (400 MHz, DMSO) δ 7.50-7.43 | 13C NMR (101 MHz, DMSO) δ |
| | (m, 5H), 7.44-7.36 (m, 5H), 4.38 (s, 2H), | 169.78, 166.61, 155.34, 153.93, |
| | 3.12 (s, 3H), 2.89 (s, 3H) | 135.19, 134.98, 130.83, 129.63, |
| | | 129.32, 129.23, 128.48, 128.40, |
| | | 37.10, 35.47, 33.9 |
| B-28 | 1H NMR (400 MHz, CDCl3) δ 7.54-7.48 | 13C NMR (101 MHz, CDCl3) δ |
| | (m, 4H), 7.46-7.29 (m, 7H), 7.18 (dd, J = | 171.43, 169.89, 155.88, 154.30, |
| | 1.8, 0.8 Hz, 1H), 6.19 (dd, J = 3.2, 1.8 Hz, | 151.08, 142.03, 134.98, 134.69, |
| | 1H), 6.10 (dq, J = 3.2, 0.7 Hz, 1H), 4.59 (q, J = | 131.24, 129.84, 129.69, 129.28, |
| | 7.4 Hz, 1H), 4.47 (ddd, J = 15.6, 5.8, 0.7 | 128.71, 128.61, 110.31, 107.18, |
| | Hz, 1H), 4.38 (ddd, J = 15.5, 5.5, 0.7 Hz, | 42.07, 36.81, 16.64 |
| | 1H), 1.69 (d, J = 7.4 Hz, 3H) | |
| B-29 | 1H NMR (400 MHz, DMSO) δ 7.50-7.35 | 13C NMR (101 MHz, DMSO) δ |
| | (m, 10H), 4.76 (q, J = 7.2 Hz, 1H), 2.77 (q, J = | 207.78, 168.89, 155.69, 154.21, |
| | 7.2 Hz, 2H), 1.55 (d, J = 7.2 Hz, 3H), 0.92 | 135.01, 134.74, 130.73, 129.55, |
| | (t, J = 7.2 Hz, 3H) | 129.24, 129.19, 128.34, 128.25, |
| | | 47.70, 32.59, 16.13, 7.62 |
| B-30 | 1H NMR (400 MHz, CDCl3) δ 7.56-7.50 | 13C NMR (101 MHz, CDCl3) δ |
| | (m, 4H), 7.44-7.29 (m, 6H), 3.80 (t, J = 6.4 | 170.39, 155.50, 153.88, 135.25, |
| | Hz, 2H), 3.56 (t, J = 6.4 Hz, 2H), 3.42 (s, | 135.15, 130.88, 129.78, 129.40, |
| | 3H) | 129.30, 128.57, 128.47, 70.82, |
| | | 58.81, 30.22 |
| B-31 | 1H NMR (600 MHz, DMSO) δ 7.51-7.35 | 13C NMR (151 MHz, DMSO) δ |
| | (m, 10H), 3.31 (t, J = 7.3 Hz, 2H), 1.80- | 170.59, 155.91, 154.33, 135.76, |
| | 1.72 (m, 2H), 1.47 (hd, J = 7.4, 1.4 Hz, 2H), | 135.56, 131.21, 130.09, 129.71, |
| | 0.93 (t, J = 7.4 Hz, 3H) | 128.90, 128.84, 31.29, 30.12, |
| | | 21.91, 13.97 |
| B-32 | 1H NMR (400 MHz, CDCl3) δ 7.57-7.49 | 13C NMR (101 MHz, CDCl3) δ |
| | (m, 8H), 7.47-7.31 (m, 12H), 7.15 (t, J = | 171.77, 171.73, 169.92, 169.85, |
| | 6.0 Hz, 1H), 7.13 (t, J = 6.0 Hz, 1H), 4.62 (q, | 155.80, 155.77, 154.24, 135.07, |
| | J = 7.3 Hz, 1H), 4.61 (q, J = 7.3 Hz, 1H), | 135.06, 134.83, 131.17, 129.87, |
| | 3.90-3.82 (m, 2H), 3.72-3.64 (m, 2H), | 129.84, 129.63, 129.30, 128.69, |
| | 3.64-3.56 (m, 2H), 3.56-3.44 (m, 2H), | 128.61, 68.08, 68.00, 43.27, 43.15, |
| | 3.29-3.18 (m, 2H), 1.90-1.68 (m, 6H), | 42.51, 42.26, 28.40, 28.36, 25.83, |
| | 1.69 (d, J = 7.3 Hz, 3H), 1.69 (d, J = 7.3 Hz, | 25.78, 16.98, 16.86 |
| | 3H), 1.50-1.38 (m, 2H) | |
| B-33 | 1H NMR (600 MHz, DMSO) δ 7.49-7.35 | 13C NMR (151 MHz, DMSO) δ |
| | (m, 10H), 4.33 (s, 2H), 2.68 (q, J = 7.3 Hz, | 204.17, 169.20, 155.44, 153.99, |
| | 2H), 0.96 (t, J = 7.3 Hz, 3H) | 135.01, 134.80, 130.72, 129.51, |
| | | 129.21, 129.16, 128.34, 128.26, |
| | | 39.75, 34.30, 7.52 |
| B-34 | 1H NMR (400 MHz, CDCl3) δ 9.41 (s, 1H), | 13C NMR (101 MHz, CDCl3) δ |
| | 8.21-8.12 (m, 2H), 7.65-7.54 (m, 3H), | 172.71, 172.14, 155.15, 142.49, |
| | 6.95-6.83 (m, 1H), 4.56 (q, J = 7.4 Hz, 1H), | 133.15, 132.60, 129.55, 127.86, |
| | 2.80 (d, J = 4.9 Hz, 3H), 1.68 (d, J = 7.4 Hz, | 42.19, 26.61, 16.82 |
| | 3H) | |
| B-36 | 1H NMR (400 MHz, DMSO) δ 7.53-7.32 | 13C NMR (101 MHz, DMSO) δ |
| | (m, 10H), 3.43 (t, J = 7.1 Hz, 2H), 2.68 (t, J = | 170.66, 155.89, 154.33, 135.74, |
| | 7.1 Hz, 2H), 2.23 (s, 6H) | 135.54, 131.22, 130.10, 129.72, |
| | | 129.70, 128.91, 128.83, 58.19, |
| | | 45.32, 28.50 |
| B-37 | 1H NMR (600 MHz, DMSO) δ 7.57-7.40 | 13C NMR (151 MHz, DMSO) δ |
| | (m, 10H), 3.37 (q, J = 7.5 Hz, 2H), 1.48 (t, J = | 170.50, 156.03, 154.39, 135.77, |
| | 7.3 Hz, 3H) | 135.58, 131.21, 130.10, 129.73, |
| | | 128.92, 128.87, 24.97, 14.94 |
| B-38 | 1H NMR (400 MHz, CDCl3) δ 7.45-7.41 | 13C NMR (101 MHz, CDCl3) δ |
| | (m, 2H), 7.41-7.38 (m, 2H), 7.19-7.15 (m, | 169.68, 169.63, 155.70, 154.02, |
| | 2H), 7.15-7.10 (m, 2H), 5.01 (q, J = 7.0 Hz, | 141.45, 139.53, 132.51, 132.37, |
| | 1H), 3.80 (dt, J = 10.0, 6.8 Hz, 1H), 3.58- | 129.75, 129.34, 129.21, 129.15, |
| | 3.46 (m, 3H), 2.38 (s, 3H), 2.36 (s, 3H), 2.01- | 46.73, 46.38, 42.02, 26.16, 24.32, |
| | 1.92 (m, 2H), 1.91-1.83 (m, 2H), 1.71 (d, | 21.50, 21.39, 18.14 |
| | J = 6.9 Hz, 3H) | |
| B-39 | 1H NMR (600 MHz, CDCl3) δ 7.55-7.48 | 13C NMR (151 MHz, CDCl3) δ |
| | (m, 8H), 7.45-7.30 (m, 12H), 5.30 (q, J = | 170.83, 170.78, 170.22, 170.03, |
| | 6.9 Hz, 1H), 5.18 (q, J = 6.9 Hz, 1H), 4.46 | 155.84, 155.84, 154.06, 154.03, |
| | (tt, J = 11.4, 3.8 Hz, 1H), 3.80 (tt, J = 11.8, | 135.22, 135.21, 135.14, 135.10, |
| | 3.7 Hz, 1H), 3.02 (s, 3H), 2.88 (s, 3H), 1.85- | 130.94, 130.91, 129.81, 129.79, |
| | 1.71 (m, 2H), 1.75 (d, J = 6.8 Hz, 3H), 1.72 | 129.49, 129.45, 129.33, 129.32, |
| | (d, J = 6.9 Hz, 3H), 1.69-1.02 (m, 18H) | 128.61, 128.51, 128.48, 57.21, |
| | | 53.23, 41.51, 41.21, 31.35, 30.86, |
| | | 29.92, 29.82, 29.60, 27.96, 25.71, |
| | | 25.65, 25.64, 25.59, 25.56, 25.26, |
| | | 19.55, 18.45 |
| B-40 | 1H NMR (600 MHz, CDCl3) δ 7.59-7.54 | 13C NMR (151 MHz, CDCl3) δ |
| | (m, 2H), 7.50-7.46 (m, 2H), 7.07 (q, J = 5.1 | 172.42, 169.22, 162.23, 160.78, |
| | Hz, 1H), 6.94-6.90 (m, 2H), 6.86-6.82 (m, | 155.21, 153.69, 131.72, 130.55, |
| | 2H), 4.56 (q, J = 7.3 Hz, 1H), 3.85 (s, 3H), | 127.66, 126.91, 114.27, 114.08, |
| | 3.83 (s, 3H), 2.80 (d, J = 4.9 Hz, 3H), 1.66 | 55.41, 55.36, 41.82, 26.59, 16.76 |
| | (d, J = 7.4 Hz, 3H) | |
| B-41 | 1H NMR (400 MHz, CDCl3) δ 8.19 (s, 1H), | 13C NMR (101 MHz, ) δ 156.76, |
| | 7.65-7.57 (m, 2H), 7.58-7.46 (m, 2H), | 156.56, 153.87, 152.00, 135.32, |
| | 7.47-7.27 (m, 6H), 3.90 (s, 3H) | 135.16, 131.05, 130.01, 129.35, |
| | | 129.33, 128.62, 128.54, 53.11 |
| B-42 | 1H NMR (400 MHz, CDCl3) δ 7.56-7.53 | 13C NMR (101 MHz, CDCl3) δ |
| | (m, 2H), 7.50-7.46 (m, 2H), 7.44-7.27 (m, | 164.00, 158.47, 153.53, 135.46, |
| | 6H), 4.77 (t, J = 5.9 Hz, 2H), 2.88 (t, J = 5.9 | 135.22, 130.85, 129.86, 129.35, |
| | Hz, 2H), 2.38 (s, 6H) | 129.09, 128.52, 128.38, 66.43, |
| | | 57.84, 45.89 |
| B-43 | 1H NMR (400 MHz, CDCl3) δ 7.69-7.65 | 13C NMR (101 MHz, CDCl3) δ |
| | (m, 2H), 7.64-7.48 (m, 8H), 6.70-6.60 (m, | 169.83, 160.04, 157.19, 150.83, |
| | 1H), 6.39-6.30 (m, 1H), 4.52 (d, J = 5.8 Hz, | 136.01, 135.79, 130.57, 129.65, |
| | 2H), 3.04 (d, J = 4.8 Hz, 3H) | 129.20, 128.65, 128.44, 128.37, |
| | | 45.31, 26.29 |
| B-44 | 1H NMR (600 MHz, CDCl3) δ 7.65-7.05 | 13C NMR (151 MHz, CDCl3) δ |
| | (m, 10H), 4.64-4.29 (m, 1H), 1.74-1.39 | 170.18, 159.57, 156.63, 154.07, |
| | (m, 3H) | 134.53, 131.27, 129.96, 129.71, |
| | | 129.35, 128.62, 128.50, 42.71, |
| | | 16.97 |
| B-45 | 1H NMR (400 MHz, CDCl3) δ 9.71-8.90 | 13C NMR (101 MHz, CDCl3) δ |
| | (m, 2H), 7.52-7.47 (m, 2H), 7.39-7.35 (m, | 172.43, 168.66, 160.69, 158.80, |
| | 2H), 7.30 (q, J = 5.0 Hz, 1H), 6.90-6.82 (m, | 155.39, 153.92, 131.75, 130.46, |
| | 2H), 6.80-6.75 (m, 2H), 4.56 (q, J = 7.3 Hz, | 126.39, 125.52, 115.86, 115.72, |
| | 1H), 2.78 (d, J = 4.7 Hz, 3H), 1.65 (d, J = | 41.96, 26.49, 17.07 |
| | 7.3 Hz, 3H) | |
| B-46 | 1H NMR (600 MHz, DMSO) δ 7.53-7.34 | 13C NMR (151 MHz, DMSO) δ |
| | (m, 10H), 5.10 (t, J = 5.5 Hz, 1H), 3.77 (q, J = | 170.46, 155.97, 154.43, 135.75, |
| | 6.3 Hz, 2H), 3.42 (t, J = 6.6 Hz, 2H) | 135.53, 131.23, 130.13, 129.74, |
| | | 129.72, 128.92, 128.87, 60.11, |
| | | 33.25 |
| B-47 | 1H NMR (600 MHz, CDCl3) δ 9.20-9.01 | 13C NMR (151 MHz, CDCl3) δ |
| | (m, 1H), 7.61-7.57 (m, 2H), 7.54-7.50 (m, | 174.28, 156.96, 156.82, 153.71, |
| | 2H), 7.46-7.30 (m, 6H), 2.96 (q, J = 7.4 Hz, | 135.37, 135.20, 131.22, 130.07, |
| | 2H), 1.30 (t, J = 7.4 Hz, 3H) | 129.50, 129.38, 128.76, 128.66, |
| | | 31.10, 9.06 |
| B-48 | 1H NMR (400 MHz, CDCl3) δ 7.52-7.47 | 13C NMR (101 MHz, CDCl3) δ |
| | (m, 4H), 7.46-7.29 (m, 6H), 7.07 (dd, J = | 171.29, 169.87, 155.92, 154.29, |
| | 5.0, 1.4 Hz, 1H), 6.85-6.83 (m, 1H), 6.81 | 140.80, 134.97, 134.68, 131.24, |
| | (dd, J = 5.0, 3.5 Hz, 1H), 4.66-4.55 (m, | 129.84, 129.69, 129.28, 128.70, |
| | 3H), 1.70 (d, J = 7.3 Hz, 3H) | 128.61, 126.74, 125.72, 124.95, |
| | | 42.03, 38.54, 16.54 |
| B-49 | 1H NMR (600 MHz, DMSO) δ 7.54-7.25 | 13C NMR (151 MHz, DMSO) δ |
| | (m, 15H), 4.61 (s, 2H) | 170.06, 156.09, 154.65, 137.72, |
| | | 135.69, 135.45, 131.27, 130.16, |
| | | 129.78, 129.75, 129.56, 128.96, |
| | | 128.92, 128.86, 127.81, 34.48 |
| B-50 | 1H NMR (600 MHz, CDCl3) δ 7.50-7.47 | 13C NMR (151 MHz, CDCl3) δ |
| | (m, 2H), 7.44-7.41 (m, 2H), 7.41-7.37 (m, | 160.46, 156.58, 149.54, 136.47, |
| | 1H), 7.33-7.29 (m, 5H), 5.69-5.52 (m, | 136.41, 130.16, 129.57, 129.18, |
| | 1H), 3.71 (q, J = 5.0 Hz, 2H), 1.62 (q, J = | 128.27, 128.21, 41.74, 34.46, 8.64, |
| | 7.0 Hz, 2H), 0.85-0.74 (m, 1H), 0.54-0.46 | 4.30 |
| | (m, 2H), 0.18-0.11 (m, 2H) | |
| B-51 | 1H NMR (400 MHz, CDCl3) δ 7.49-7.45 | 13C NMR (101 MHz, CDCl3) δ |
| | (m, 2H), 7.44-7.39 (m, 3H), 7.36-7.29 (m, | 159.36, 156.94, 150.67, 136.00, |
| | 5H), 5.33 (h, J = 6.7 Hz, 1H), 5.06 (t, J = 7.1 | 135.97, 130.51, 129.58, 129.19, |
| | Hz, 2H), 4.74-4.66 (m, 2H). | 128.57, 128.38, 78.76, 46.57 |
| B-52 | 1H NMR (400 MHz, CDCl3) δ 7.53-7.27 | 13C NMR (101 MHz, CDCl3) δ |
| | (m, 10H), 6.64 (q, J = 3.1 Hz, 1H), 4.89 (s, | 167.48, 166.50, 153.76, 143.55, |
| | 2H), 2.86 (d, J = 4.9 Hz, 3H) | 134.77, 133.56, 131.77, 129.96, |
| | | 129.55, 129.04, 128.50, 128.28, |
| | | 57.23, 26.49 |
| B-53 | 1H NMR (400 MHz, CDCl3) δ 7.51-7.27 | 13C NMR (101 MHz, CDCl3) δ |
| | (m, 10H), 6.84-6.72 (m, 1H), 4.85 (s, 2H), | 167.44, 167.16, 153.72, 143.49, |
| | 2.77 (tq, J = 7.2, 3.7 Hz, 1H), 0.81-0.73 (m, | 134.77, 133.59, 131.76, 129.97, |
| | 2H), 0.61-0.54 (m, 2H) | 129.53, 129.05, 128.50, 128.27, |
| | | 57.20, 22.90, 6.50 |
| B-54 | 1H NMR (400 MHz, CDCl3) δ 7.49-7.45 | 13C NMR (151 MHz, CDCl3) δ |
| | (m, 2H), 7.42-7.37 (m, 3H), 7.34-7.27 (m, | 160.75, 156.80, 149.51, 136.22, |
| | 5H), 3.97 (p, J = 5.2 Hz, 1H), 3.87-3.76 (m, | 136.13, 130.26, 129.53, 129.08, |
| | 1H), 3.76-3.60 (m, 3H) | 128.28, 128.24, 71.25, 71.05, |
| | | 64.92, 64.03, 44.26, 43.98 |
| B-55 | 1H NMR (600 MHz, CDCl3) δ 7.49-7.46 | 13C NMR (151 MHz, CDCl3) δ |
| | (m, 2H), 7.44-7.41 (m, 2H), 7.41-7.37 (m, | 160.32, 156.70, 149.82, 136.35, |
| | 1H), 7.33-7.28 (m, 5H), 5.87-5.77 (m, | 136.32, 130.21, 129.57, 129.19, |
| | 1H), 3.83 (q, J = 5.0 Hz, 2H), 3.66 (t, J = 5.2 | 128.27, 71.04, 58.86, 41.13 |
| | Hz, 2H), 3.42 (s, 3H) | |
| B-56 | 1H NMR (400 MHz, CDCl3) δ 8.99-8.55 | 13C NMR (101 MHz, CDCl3) δ |
| | (m, 1H), 7.89 (s, 1H), 7.53-7.32 (m, 10H), | 157.52, 156.52, 154.28, 153.23, |
| | 5.74-5.34 (m, 1H) | 135.01, 134.79, 131.28, 129.69, |
| | | 129.46, 129.27, 128.67, 128.65 |
| B-57 | 1H NMR (400 MHz, CDCl3) δ 7.53-7.49 | 13C NMR (101 MHz, CDCl3) δ |
| | (m, 2H), 7.49-7.42 (m, 1H), 7.41-7.35 (m, | 169.09, 166.26, 153.92, 143.13, |
| | 1H), 7.35-7.28 (m, 6H), 6.74 (q, J = 3.3 Hz, | 134.82, 133.76, 131.71, 129.96, |
| | 1H), 5.45 (q, J = 7.1 Hz, 1H), 2.83 (d, J = | 129.46, 129.05, 128.46, 128.29, |
| | 4.9 Hz, 3H), 1.75 (d, J = 7.1 Hz, 3H) | 58.83, 26.52, 14.66 |
| B-58 | 1H NMR (400 MHz, CDCl3) δ 7.51-7.45 | 13C NMR (101 MHz, CDCl3) δ |
| | (m, 2H), 7.43-7.36 (m, 3H), 7.35-7.27 (m, | 161.18, 157.73, 149.15, 136.29, |
| | 5H), 3.93 (t, J = 4.5 Hz, 2H), 3.75 (q, J = 5.0 | 136.06, 130.54, 129.76, 129.19, |
| | Hz, 2H) | 128.55, 128.43, 61.85, 44.68. |
| B-59 | 1H NMR (400 MHz, CDCl3) δ 8.49 (d, J = | 13C NMR (101 MHz, CDCl3) δ |
| | 1.6 Hz, 1H), 8.37 (dd, J = 2.8, 1.6 Hz, 1H), | 172.37, 155.94, 144.07, 143.62, |
| | 8.28 (d, J = 2.7 Hz, 1H), 7.05-6.67 (m, 1H), | 140.19, 41.23, 26.55, 17.00 |
| | 4.36 (q, J = 7.4 Hz, 1H), 2.79 (d, J = 4.9 Hz, | |
| | 3H), 1.61 (d, J = 7.4 Hz, 3H) | |
| B-60 | 1H NMR (400 MHz, CDCl3) δ 7.14-6.91 | 13C NMR (101 MHz, CDCl3) δ |
| | (m, 1H), 4.48 (q, J = 7.4 Hz, 1H), 2.79 (d, J = | 172.35, 170.18, 159.43, 154.12, |
| | 4.8 Hz, 3H), 2.63 (s, 3H), 2.50 (s, 3H), | 41.58, 26.51, 21.77, 19.15, 16.68 |
| | 1.61 (d, J = 7.4 Hz, 3H) | |
| B-61 | 1H NMR (400 MHz, CDCl3) δ 7.25-7.20 | 13C NMR (101 MHz, CDCl3) δ |
| | (m, 2H), 7.18 (d, J = 1.9 Hz, 1H), 7.06 (dd, J = | 170.48, 169.26, 154.77, 153.78, |
| | 8.3, 2.0 Hz, 1H), 6.86 (d, J = 8.4 Hz, 1H), | 151.86, 150.26, 149.21, 148.90, |
| | 6.84-6.77 (m, 2H), 4.32 (t, J = 7.3 Hz, 1H), | 127.93, 126.99, 123.86, 122.34, |
| | 4.18 (h, J = 6.7 Hz, 1H), 3.92 (s, 3H), 3.90 | 112.51, 112.01, 111.09, 110.68, |
| | (s, 3H), 3.81 (s, 3H), 3.76 (s, 3H), 2.20 (dp, | 56.02, 55.99, 55.96, 51.37, 49.75, |
| | J = 14.5, 7.2 Hz, 1H), 1.96 (dp, J = 14.6, 7.4 | 33.00, 32.79, 24.45, 23.65, 23.62, |
| | Hz, 1H), 1.91-1.82 (m, 2H), 1.54-1.45 (m, | 12.14 |
| | 4H), 1.34-1.21 (m, 2H), 1.13 (t, J = 7.4 Hz, | |
| | 3H) | |
| B-62 | 1H NMR (400 MHz, CDCl3) δ 7.50-7.44 | 13C NMR (101 MHz, CDCl3) δ |
| | (m, 2H), 7.44-7.40 (m, 2H), 7.21-7.17 (m, | 169.77, 169.01, 155.85, 154.47, |
| | 2H), 7.17-7.12 (m, 2H), 7.10-7.05 (m, | 142.00, 139.89, 132.20, 131.90, |
| | 1H), 3.90 (s, 2H), 2.70 (tq, J = 7.2, 3.7 Hz, | 129.82, 129.46, 129.38, 129.14, |
| | 1H), 2.39 (s, 3H), 2.37 (s, 3H), 0.75-0.65 | 34.08, 22.84, 21.54, 21.41, 6.51 |
| | (m, 2H), 0.47-0.35 (m, 2H) | |
| B-63 | 1H NMR (600 MHz, CDCl3) δ 7.48-7.45 | 13C NMR (151 MHz, CDCl3) δ |
| | (m, 2H), 7.44-7.41 (m, 2H), 7.41-7.38 (m, | 173.87, 159.26, 156.88, 150.42, |
| | 1H), 7.34-7.28 (m, 5H), 6.04-5.89 (m, | 136.16, 136.08, 130.35, 129.61, |
| | 1H), 4.94-4.84 (m, 1H), 3.79 (s, 3H), 1.62 | 129.22, 128.44, 128.32, 128.28, |
| | (d, J = 7.2 Hz, 3H) | 52.49, 49.91, 18.43 |
| B-64 | 1H NMR (600 MHz, DMSO) δ 7.52-7.35 | 13C NMR (151 MHz, DMSO) δ |
| | (m, 10H), 2.71 (s, 3H) | 170.85, 155.91, 154.33, 135.76, |
| | | 135.56, 131.23, 130.13, 129.74, |
| | | 129.73, 128.93, 128.86, 13.76 |
| B-65 | 1H NMR (600 MHz, CDCl3) δ 7.56-7.52 | 13C NMR (151 MHz, CDCl3) δ |
| | (m, 2H), 7.52-7.49 (m, 2H), 7.47-7.40 (m, | 171.45, 170.14, 155.93, 154.30, |
| | 2H), 7.40-7.36 (m, 2H), 7.36-7.31 (m, | 135.06, 134.78, 131.22, 129.88, |
| | 2H), 6.77 (q, J = 4.6 Hz, 1H), 4.41 (d, J = | 129.66, 129.28, 128.73, 128.61, |
| | 7.1 Hz, 1H), 2.81 (d, J = 4.8 Hz, 3H), 2.60- | 55.54, 29.77, 26.58, 21.13, 19.92 |
| | 2.50 (m, J = 6.8 Hz, 1H), 1.17 (d, J = 6.7 Hz, | |
| | 3H), 1.16 (d, J = 6.8 Hz, 3H) | |
| B-66 | 1H NMR (600 MHz, CDCl3) δ 7.69 (dd, J = | 13C NMR (151 MHz, CDCl3) δ |
| | 2.9, 1.4 Hz, 1H), 7.60-7.55 (m, 3H), 7.55- | 172.33, 172.19, 170.11, 169.61, |
| | 7.46 (m, 6H), 7.44-7.39 (m, 2H), 7.31 (dd, | 155.83, 153.84, 150.44, 150.06, |
| | J = 5.1, 3.0 Hz, 1H), 7.24 (dd, J = 5.1, 2.9 | 136.32, 135.67, 135.43, 134.94, |
| | Hz, 1H), 7.23 (dd, J = 5.1, 1.4 Hz, 1H), 7.14 | 132.19, 131.33, 129.95, 129.42, |
| | (dd, J = 5.1, 1.3 Hz, 1H), 7.01 (q, J = 5.0 Hz, | 129.09, 128.99, 128.75, 128.20, |
| | 1H), 6.97 (q, J = 5.0 Hz, 1H), 4.57 (d, J = | 127.69, 127.28, 126.27, 126.24, |
| | 7.4 Hz, 1H), 4.54 (q, J = 7.4 Hz, 1H), 2.80 | 42.22, 42.02, 26.64, 26.62, 16.86, |
| | (d, J = 4.9 Hz, 6H), 1.68 (d, J = 7.4 Hz, 3H), | 16.82 |
| | 1.67 (d, J = 7.4 Hz, 3H) | |
| B-67 | 1H NMR (600 MHz, CDCl3) δ 7.79 (dd, J = | 13C NMR (151 MHz, CDCl3) δ |
| | 2.8, 1.5 Hz, 1H), 7.69 (dd, J = 3.0, 1.3 Hz, | 172.27, 169.75, 150.47, 149.70, |
| | 1H), 7.42 (dd, J = 5.0, 3.0 Hz, 1H), 7.31 (dd, | 136.28, 135.89, 131.71, 128.00, |
| | J = 5.1, 2.8 Hz, 1H), 7.29 (dd, J = 5.1, 1.5 | 127.73, 127.01, 126.70, 126.42, |
| | Hz, 1H), 7.22 (dd, J = 5.0, 1.3 Hz, 1H), 6.98 | 42.25, 26.64, 16.84 |
| | (q, J = 4.2 Hz, 1H), 4.52 (q, J = 7.4 Hz, 1H), | |
| | 2.80 (d, J = 4.8 Hz, 3H), 1.67 (d, J = 7.4 Hz, | |
| | 3H) | |
| B-68 | 1H NMR (600 MHz, CDCl3) δ 7.57-7.53 | 13C NMR (151 MHz, CDCl3) δ |
| | (m, 2H), 7.52-7.48 (m, 2H), 7.46-7.41 (m, | 172.27, 170.14, 170.06, 156.30, |
| | 4H), 7.40-7.36 (m, 2H), 7.36-7.31 (m, | 156.08, 154.49, 154.43, 138.72, |
| | 2H), 7.26-7.20 (m, 4H), 7.19-7.15 (m, | 138.59, 135.08, 134.89, 134.75, |
| | 2H), 7.07-7.00 (m, 2H), 4.59 (q, J = 7.4 Hz, | 134.60, 132.05, 131.24, 130.47, |
| | 1H), 4.59 (q, J = 7.4 Hz, 1H), 2.81 (d, J = | 130.40, 129.85, 129.74, 129.66, |
| | 4.9 Hz, 3H), 2.81 (d, J = 4.8 Hz, 3H), 2.35 | 129.25, 128.67, 128.55, 128.48, |
| | (s, 3H), 2.31 (s, 3H), 1.68 (d, J = 7.4 Hz, 6H) | 128.34, 127.01, 126.47, 45.53, |
| | | 41.92, 41.91, 26.61, 21.41, 21.36, |
| | | 16.80 |
| B-69 | 1H NMR (600 MHz, CDCl3) δ 8.55-8.50 | 13C NMR (151 MHz, CDCl3) δ |
| | (m, 2H), 7.86-7.81 (m, 2H), 7.60-7.52 (m, | 171.46, 162.37, 160.24, 155.65, |
| | 6H), 6.55 (q, J = 4.0 Hz, 1H), 4.58 (q, J = | 134.37, 133.57, 131.92, 130.65, |
| | 7.4 Hz, 1H), 2.77 (d, J = 4.8 Hz, 3H), 1.66 | 129.08, 128.90, 128.80, 128.22, |
| | (d, J = 7.4 Hz, 3H) | 42.40, 26.75, 16.90 |
| B-70 | 1H NMR (600 MHz, CDCl3) δ 7.61 (dd, J = | 13C NMR (151 MHz, CDCl3) δ |
| | 5.0, 1.1 Hz, 1H), 7.58 (dd, J = 5.1, 1.1 Hz, | 172.02, 169.56, 149.35, 147.24, |
| | 1H), 7.49 (dd, J = 3.9, 1.1 Hz, 1H), 7.41 (dd, | 138.32, 136.18, 133.60, 132.83, |
| | J = 3.6, 1.1 Hz, 1H), 7.15 (dd, J = 5.1, 3.6 | 129.39, 129.26, 128.59, 127.78, |
| | Hz, 1H), 7.03 (dd, J = 5.0, 3.9 Hz, 1H), 6.95 | 42.08, 26.64, 16.86 |
| | (q, J = 5.3 Hz, 1H), 4.51 (q, J = 7.4 Hz, 1H), | |
| | 2.81 (d, J = 4.8 Hz, 3H), 1.67 (d, J = 7.4 Hz, | |
| | 3H) | |
| B-71 | 1H NMR (600 MHz, CDCl3) δ 7.55-7.52 | 13C NMR (151 MHz, CDCl3) δ |
| | (m, 2H), 7.52-7.50 (m, 2H), 7.46-7.41 (m, | 171.85, 170.30, 156.03, 154.30, |
| | 2H), 7.39-7.36 (m, 2H), 7.35-7.32 (m, | 135.03, 134.73, 131.23, 129.85, |
| | 2H), 6.90 (q, J = 5.5 Hz, 1H), 4.50 (t, J = 7.5 | 129.68, 129.25, 128.73, 128.60, |
| | Hz, 1H), 2.81 (d, J = 4.9 Hz, 3H), 2.16 | 47.32, 32.91, 26.58, 20.71, 13.76 |
| | (dddd, J = 13.6, 9.8, 7.4, 5.9 Hz, 1H), 1.89 | |
| | (dddd, J = 13.9, 9.9, 7.6, 5.5 Hz, 1H), 1.63- | |
| | 1.49 (m, 2H), 0.98 (t, J = 7.4 Hz, 3H) | |
| B-72 | 1H NMR (600 MHz, CDCl3) δ 7.57-7.53 | 13C NMR (151 MHz, CDCl3) δ |
| | (m, 2H), 7.53-7.49 (m, 2H), 7.47-7.41 (m, | 172.45, 170.24, 155.91, 154.33, |
| | 2H), 7.41-7.36 (m, 2H), 7.36-7.32 (m, | 134.99, 134.78, 131.26, 129.84, |
| | 2H), 6.99 (d, J = 3.3 Hz, 1H), 4.32 (t, J = 7.4 | 129.70, 129.30, 128.74, 128.65, |
| | Hz, 1H), 2.71 (tq, J = 7.2, 3.6 Hz, 1H), 2.20 | 49.13, 24.35, 22.81, 12.09, 6.80, |
| | (dp, J = 14.1, 7.4 Hz, 1H), 1.95 (dp, J = | 6.37 |
| | 14.1, 7.3 Hz, 1H), 1.12 (t, J = 7.3 Hz, 3H), | |
| | 0.76-0.64 (m, 2H), 0.41-0.35 (m, 2H) | |
| B-73 | 1H NMR (600 MHz, CDCl3) δ 8.51 (s, 1H), | 13C NMR (151 MHz, CDCl3) δ |
| | 7.45-7.29 (m, 10H), 7.18-7.14 (m, 1H), | 172.71, 152.78, 151.20, 148.29, |
| | 4.12 (t, J = 7.4 Hz, 1H), 2.63 (tq, J = 7.4, 3.8 | 141.04, 138.19, 137.78, 129.57, |
| | Hz, 1H), 2.17 (dp, J = 14.6, 7.4 Hz, 1H), | 129.40, 129.11, 128.63, 128.56, |
| | 1.90 (dp, J = 14.5, 7.3 Hz, 1H), 1.10 (t, J = | 128.35, 48.32, 24.12, 22.57, 12.13, |
| | 7.4 Hz, 3H), 0.56 (dtd, J = 9.7, 6.6, 5.0 Hz, | 6.40, 5.72 |
| | 1H), 0.49 (dddd, J = 9.8, 7.3, 6.4, 5.1 Hz, | |
| | 1H), 0.05 (dddd, J = 10.2, 6.4, 5.0, 3.8 Hz, | |
| | 1H),-0.06 (dddd, J = 10.4, 6.6, 5.1, 3.9 Hz, | |
| | 1H) | |
| B-74 | 1H NMR (600 MHz, CDCl3) δ 8.50 (s, 1H), | 13C NMR (151 MHz, CDCl3) δ |
| | 7.46-7.41 (m, 4H), 7.41-7.28 (m, 7H), | 173.18, 152.72, 151.19, 148.37, |
| | 4.33 (q, J = 7.4 Hz, 1H), 2.62 (tq, J = 7.5, | 140.92, 138.12, 137.73, 129.58, |
| | 3.8 Hz, 1H), 1.62 (d, J = 7.4 Hz, 3H), 0.54 | 129.39, 129.17, 128.68, 128.59, |
| | (dddd, J = 9.7, 7.1, 6.7, 5.1 Hz, 1H), 0.46 | 128.38, 40.93, 22.57, 16.51, 6.38, |
| | (dddd, J = 9.7, 7.2, 6.6, 5.2 Hz, 1H), 0.03 | 5.58 |
| | (dddd, J = 10.3, 6.5, 5.1, 3.8 Hz, 1H),-0.13 | |
| | (dddd, J = 10.4, 6.6, 5.2, 3.9 Hz, 1H) | |
| B-75 | 1H NMR (300 MHz, DMSO-d6) δ 8.62 (s, | |
| | 1H), 8.22 (d, J = 4.9 Hz, 1H), 7.47-7.28 (m, | |
| | 10H), 4.38 (dd, J = 7.9, 6.1 Hz, 1H), 2.60 (d, | |
| | J = 4.6 Hz, 3H), 2.01-1.81 (m, 2H), 0.95 (t, | |
| | J = 7.3 Hz, 3H) | |
| B-76 | 1H NMR (600 MHz, CDCl3) δ 7.69 (dd, J = | 13C NMR (151 MHz, CDCl3) δ |
| | 2.9, 1.4 Hz, 1H), 7.60-7.55 (m, 3H), 7.55- | 172.33, 172.19, 170.11, 169.61, |
| | 7.46 (m, 6H), 7.44-7.39 (m, 2H), 7.31 (dd, | 155.83, 153.84, 150.44, 150.06, |
| | J = 5.1, 3.0 Hz, 1H), 7.24 (dd, J = 5.1, 2.9 | 136.32, 135.67, 135.43, 134.94, |
| | Hz, 1H), 7.23 (dd, J = 5.1, 1.4 Hz, 1H), 7.14 | 132.19, 131.33, 129.95, 129.42, |
| | (dd, J = 5.1, 1.3 Hz, 1H), 7.01 (q, J = 5.0 Hz, | 129.09, 128.99, 128.75, 128.20, |
| | 1H), 6.97 (q, J = 5.0 Hz, 1H), 4.57 (d, J = | 127.69, 127.28, 126.27, 126.24, |
| | 7.4 Hz, 1H), 4.54 (q, J = 7.4 Hz, 1H), 2.80 | 42.22, 42.02, 26.64, 26.62, 16.86, |
| | (d, J = 4.9 Hz, 6H), 1.68 (d, J = 7.4 Hz, 3H), | 16.82 |
| | 1.67 (d, J = 7.4 Hz, 3H) | |
| B-77 | 1H NMR (600 MHz, CDCl3) δ 7.57-7.53 | 13C NMR (151 MHz, CDCl3) δ |
| | (m, 2H), 7.52-7.48 (m, 2H), 7.46-7.41 (m, | 172.27, 170.14, 170.06, 156.30, |
| | 4H), 7.40-7.36 (m, 2H), 7.36-7.31 (m, | 156.08, 154.49, 154.43, 138.72, |
| | 2H), 7.26-7.20 (m, 4H), 7.19-7.15 (m, | 138.59, 135.08, 134.89, 134.75, |
| | 2H), 7.07-7.00 (m, 2H), 4.59 (q, J = 7.4 Hz, | 134.60, 132.05, 131.24, 130.47, |
| | 1H), 4.59 (q, J = 7.4 Hz, 1H), 2.81 (d, J = | 130.40, 129.85, 129.74, 129.66, |
| | 4.9 Hz, 3H), 2.81 (d, J = 4.8 Hz, 3H), 2.35 | 129.25, 128.67, 128.55, 128.48, |
| | (s, 3H), 2.31 (s, 3H), 1.68 (d, J = 7.4 Hz, 6H) | 128.34, 127.01, 126.47, 45.53, |
| | | 41.92, 41.91, 26.61, 21.41, 21.36, |
| | | 16.80 |

### Formulation Examples

### Formulation Examples for Cosmetic Preparations

The following formulation examples F1 to F54 show a wide variety of formulations for cosmetic and pharmaceutical preparations. Coolant 1 here means compound B-11 according to the invention, coolant 2 means compound A-02, coolant 3 means compound A-09, coolant 4 means compound A-10. The coolants were used in pure form.

In the following tables, the decimal point is shown as a dot.

TABLE F1
Transparent liquid soap (quantities in % by weight)

| Components | INCI | Quantity |
|---|---|---|
| Tagat O 2 | PEG-20 Glyceryl Oleate | 2.5 |
| Coconut oil diethanolamine | Cocamide DEA | 5.0 |
| condensate | | |
| Abil B 8842 | Cyclomethicone | 0.5 |
| Sodium lauryl ether sulfate, 28% | Sodium Laureth Sulfate | 35.0 |
| Tego-Betaine L7 | Cocamidopropyl Betaine | 5.0 |
| Soap, 25% | Coconut acid, Potassium salt, | 20.0 |
| | Potassium oleate | |
| Hydrolite ® 5 | 1,2-Propanediol | 0.4 |
| Coolant 1 | | 1.0 |
| Water | Water | ad 100 |

TABLE F2
Liquid detergent soap (quantities in % by weight)

| Components | INCI | Quantity |
|---|---|---|
| Elfan OS 46 | Sodium Olefin C14-C16 Sulfonate | 35.5 |
| Armoteric LB | Lauryl Betaine | 8.0 |
| Elfan SG | | 10.0 |
| Elfacos GT 282 L | Talloweth-60 Myristyl Glycol | 3.0 |
| PCL-Liquid 100 | Cetearyl ethylhexanoate | 4.0 |
| Hydrolite ® 5 Green | Hydrolite® 5 Green | 0.4 |
| Preservative | Methylchloroisothiazolinone | 0.1 |
| Coolant 2 | | 1.5 |
| Water | Water | ad 100 |

TABLE F3
Body care lotion (quantities in % by weight)

| Components | INCI | Quantity |
|---|---|---|
| Lumerol K 28 | Disodium Laureth Sulfosuccinate, | 33.0 |
| | Cocamidopropyl Betaine, | |
| | Magnesium Lauryl Sulfate | |
| Amphoteric surfactant B 4 | Cocamidopropyl Betaine | 10.0 |
| Pearlescent agent GM 4055 | MIPA-Pareth-25 Sulfate, Glycol | 4.0 |
| | Stearate | |
| Sodium Chloride | Sodium Chloride | 2.0 |
| Avocado oil | Persea Gratissima (Avocado) Oil | 3.0 |
| Water | Water | ad 100 |
| Hydrolite ® 5 | Hydrolite ® 5 Green | 0.5 |
| Euxyl® K727 | Phenoxyethanol, Methyldibromo | 0.3 |
| | Glutaronitrile, Isothiazolinones | |
| Coolant 3 | | 1.0 |

TABLE F4
Body care lotion with triclosan (amounts in % by weight)

| Components | INCI | Quantity |
|---|---|---|
| Texapon N 25 | Sodium Laureth Sulfate | 37.5 |
| Lamepon S | Potassium Cocoyl Hydrolyzed | 28.0 |
| | Collagen | |
| Lamesoft LMG | Hydrogenated Tallow Glycerides, | 5.0 |
| | TEA-Cocoyl Hydrolyzed Collagen | |
| Lamesoft 156 | Glyceryl Laurate, TEA-Cocoyl | 5.0 |
| | Hydrolyzed Collagen | |
| Sodium Chloride | Sodium Chloride | 1.7 |
| Irgasan DP 300 | Triclosan | 0.5 |
| Water | Water | ad 100 |
| Hydrolite ® 5 | Hydrolite ® 5 Green | 0.3 |
| Euxyl ® K703 | Phenoxyethanol, Benzoic Acid, | 0.4 |
| | Dehydroacetic Acid | |
| Coolant 4 | | 1.0 |

TABLE F5
Intimate wash (quantities in % by weight)

| Components | INCI | Quantity |
|---|---|---|
| Tegobetaine HS | Cocamidopropyl Betaine, Glyceryl | 15.0 |
| | Laurate | |
| Tagat L 2 | PEG-20 Glyceryl Laurate | 2.0 |
| Arlacide G | Chlorhexidine Digluconate | 0.1 |
| Rewoquat B 50 | Benzalkonium Chloride | 0.1 |
| Lactic Acid, 80% | Lactic Acid | 0.1 |
| Water | Water | ad 100 |
| Hydrolite ® 5 | Hydrolite ® 5 Green | 0.2 |
| Euxyl ® K700 | Potassium Sorbate, Benzyl Alcohol, | 0.3 |
| | Phenoxyethanol | |
| Coolant 1 | | 1.3 |

TABLE F6
Liquid soap (quantities in % by weight)

| Components | INCI | Quantity |
|---|---|---|
| Deionized water | Water | 2.0 |
| Soap bases mix | Sodium tallowates/palmitates | 94.3 |
| Titanium dioxide | Titanium dioxide | 1.0 |
| Hydrolite ® 5 | Hydrolite ® 5 Green | 1.2 |
| PreservativePreservatives | Phenoxyethanol | 0.5 |
| Coolant 2 | | 1.0 |

TABLE F7
Shampoo (quantities in % by weight)

| Ingredients (INCI) | Quantity |
|---|---|
| Sodium lauryl ether sulfate (e.g. Texapon NSO) | 12 |
| Cocamidopropyl betaine (e.g. Dehyton K) | 2 |
| Sodium chloride | 1.4 |
| Citric acid | 1.3 |
| 1,2-Pentanediol (Hydrolite ® 5) | 0.3 |
| Phenoxyethanol, methyl-, ethyl-, butyl- and propylparaben | 0.5 |
| Coolant 2 | 1.0 |
| Water | ad 100 |

TABLE F8
2-in-1 shampoo (quantities in % by weight)

| Components | INCI | Quantity |
|---|---|---|
| Deionized water | Water | ad 100 |
| Plantacare PS 10 | Sodium Laureth Sulfate, Lauryl | 20.0 |
| | Glucoside | |
| Euperlan PK 771 | Glycol Distearate, Sodium Lauryl | 6.0 |
| | Sulfate, Cocamide | |
| | MEA, Laureth-10 | |
| Sodium chloride | Sodium Chloride | 1.4 |
| Citric acid monohydrate | Citric acid | 0.1 |
| crystalline | | |
| Hydrolite ® 5 | Hydrolite ® 5 Green | 0.5 |
| Dragocid Liquid | Phenoxyethanol, Parabens | 0.5 |
| Coolant 3 | | 1.0 |

TABLE F9
Anti-dandruff shampoo (quantities in % by weight)

| Ingredients (INCI) | Quantity |
|---|---|
| Climbazole | 0.50 |
| Sodium Laureth Sulfate | 37.00 |
| Cocamidopropyl Betaine | 8.00 |
| PEG-6 Caprylic/Capric Glycerides | 2.50 |
| Laureth-2 | 2.00 |
| Water (Aqua), Glycerol, Thymus Vulgaris | 0.50 |
| (Thyme), Flower/Leaf Extract | |
| Rosmarinus Officinalis (Rosemary) Leaf | 0.50 |
| Water, Water (Aqua), Butylene | |
| Glycol, Pentylene Glycol | |
| Bisabolol | 0.10 |
| Panthenol | 0.50 |
| Polyquaternium-10 | 0.40 |
| 1,2-Pentanediol (Hydrolite ® 5) | 0.50 |
| Phenoxyethanol, methylparaben, | 0.70 |
| ethylparaben, butylparaben, | |
| propylparaben, isobutylparaben | |
| Coolant 4 | 1.50 |
| Water (Aqua) | ad 100 |

TABLE F10
Hair conditioner with Crinipan, rinse-off (amounts in % by weight)

| Components | INCI | Quantity |
|---|---|---|
| Lanette ® O | Cetearyl Alcohol | 4.00 |
| Dragoxat 89 | Ethylhexyl Isononanoate | 2.00 |
| Genamin ® KDM-P | Behentrimonium Chloride | 1.00 |
| SF 1550 | Phenyl Trimethicone | 0.10 |
| Neo Heliopan ® BB | Benzophenone-3 | 0.10 |
| Crinipan ® AD | Climbazole | 0.80 |
| Glycerol 99.5 P. | Glycerol | 6.00 |
| Water | Water (Aqua) | ad 100 |
| Actipone ® | Water (Aqua), Butylene | 0.50 |
| Alpha Pulp | Glycol, Malic Acid, | |
| | Actinidia Chinensis (Kiwi) | |
| | Fruit Juice, Citrus, | |
| | Aurantium Dulcis | |
| | (Orange), Juice, Citrus | |
| | Paradisi (Grapefruit) | |
| | Juice, Pyrus Malus | |
| | (Apple) Juice, Trideceth-9, Prunus | |
| | Amygdalus Dulcis | |
| | (Sweet Almond) Seed | |
| | Extract | |
| Extrapone ® | Propylene Glycol, | 0.50 |
| Bamboo P | Water (Aqua), Butylene | |
| | Glycol, Bambusa Vulgaris | |
| | Shoot Extract | |
| Sodium | Sodium hydroxide | 0.40 |
| hydroxide 10% | | |
| solution | | |
| Color I | Color | 0.60 |
| Color II | Color | 0.30 |
| Hydrolite ® 5 | Hydrolite ® 5 Green | 0.40 |
| Preservative | Methylparaben | 0.20 |
| Coolant 1 | | 1.2 |

TABLE F11
Sprayable hair conditioner with zinc pyrithrione-leave-on
(amounts in % by weight)

| Components | INCI | Quantity |
|---|---|---|
| Monomuls 60-35 C | Hydrogenated Palm Glycerides | 1.70 |
| Cetiol OE | Dicaprylyl ether | 7.20 |
| Abil 100 | Dimethicone | 3.60 |
| Dehyquart F 75 | Distearoylethyl | 4.00 |
| | Hydroxyethylmonium. | |
| | Methosulfate, Cetearyl Alcohol | |
| Eumulgin B1 | Ceteareth-12 | 3.50 |
| Cetiol S | Diethylhexylcyclohexane | 7.20 |
| D-Panthenol | Panthenol | 0.10 |
| Glycerol 99.5 P. | Glycerol | 1.50 |
| Water | Water (Aqua) | ad 100 |
| Actipone ® | Water (Aqua), Propylene, | 0.10 |
| Rosemary | Glycol, Rosmarinus | |
| | Officinalis, (Rosemary) | |
| | Leaf Extract | |
| Frescolat ® ML Cryst. | Menthyl Lactate | 0.50 |
| Dragosantol 100 | Bisabolol | 0.10 |
| Zinc Omadine | Zinc pyrithione | 0.10 |
| Hydrolite ® 5 | Hydrolite ® 5 | 0.40 |
| Phenonip ® | Phenoxyethanol, methylparaben, | 0.30 |
| | ethylparaben, | |
| | butylparaben, propylparaben, | |
| | isobutylparaben | |
| Coolant 1 | | 1.50 |

TABLE F12
Hair conditioner with UV protection (quantities in % by weight)

| Components | INCI | Quantity |
|---|---|---|
| Renex PEG 6000 | PEG-150 | 2.50 |
| Hair Conditioner Base | Cetyl alcohol, | 3.00 |
| | Behentrimonium chloride, | |
| | Triticum Vulgare (Wheat) | |
| | bran extract, Linoleic | |
| | acid | |
| PCL-Solid | Stearyl heptanoate, Stearyl caprylate | 0.50 |
| Dow Corning 5200 | Laurylmethicone copolyol | 0.50 |
| Natrosol 250 HR | Hydroxyethyl cellulose | 0.50 |
| Benzophenone-4 | Benzophenone-4 | 1.00 |
| Neo Heliopan AP | Disodiumphenyldibenz-imidazole | 1.00 |
| | tetrasulphonate | |
| Amino methyl propanol | Amino methyl propanol | 2.00 |
| Dow Corning 949 | Amodimethicone, | 2.00 |
| cationic emulsion | Cetrimonium chloride, | |
| | Trideceth-12 | |
| Hydrolite ® 5 | Hydrolite ® 5 | 0.80 |
| 1,2-Hexanediol | 1,2-Hexanediol | 0.50 |
| Coolant 2 | | 1.50 |
| Water | Water (Aqua) | ad 100 |

TABLE F13
Shower gel (quantities in % by weight)

| Components | INCI | Quantity |
|---|---|---|
| Deionized water | Water | ad 100 |
| Plantacare PS 10 | Sodium Laureth Sulfate, | 20.0 |
| | Lauryl Glucoside | |
| Sodium chloride | Sodium Chloride | 1.4 |
| Citric acid monohydrate | Citric Acid | 1.3 |
| crystalline | | |
| Hydrolite ® 5 | Hydrolite ® 5 | 0.6 |
| SymDiol ® 68 | 1,2-hexanediol, caprylyl glycol | 0.4 |
| Coolant 3 | | 0.9 |

TABLE F14
Shaving foam (quantities in % by weight)

| Ingredients (INCI) | Quantity |
|---|---|
| Deionized water | ad 100 |
| Triethanolamines | 4.0 |
| Edenor L2 SM (Stearinic acid, Palmitinic acid) (Cognis) | 5.3 |
| Laureth-23 | 3.0 |
| Stearyl alcohol | 0.5 |
| Sodium lauryl sulfate | 3.0 |
| Extrapone Seaweed (Water, Propylene | 1.0 |
| glycol, Potassium iodide, Fucus | |
| Vesiculosus Extract) | |
| Dragosantol (bisabolol, farnesol) | 0.1 |
| 1,2-Pentanediol (Hydrolite ® 5) | 1.0 |
| Euxyl ® K220 (methylisothiazolinone, ethylhexylglyerol) | 0.6 |
| Coolant 4 | 0.6 |
| Propane, Butane 4.2 Bar | 4.0 |

TABLE F15
Depilatory cream (quantities in % by weight)

| Ingredients (INCI) | Quantity |
|---|---|
| Cetearyl alcohol | 10.0 |
| Ceteareth-12 | 2.0 |
| PCL-Liquid (Cetearylethylhexanoate, Isopropylmyristate) | 3.0 |
| Dragosantol (bisabolol, farnesol) | 0.1 |
| Edenor L2 SM (Stearinic acid, Palmitinic acid) | 1.0 |
| Dem. Water | 52.2 |
| Urea | 5.0 |
| Deionized water | ad 100 |
| Calcium thioglycolate | 6.0 |
| Sodium hydroxide solution, 10 % | 10.0 |
| 1,2-Pentanediol (Hydrolite ® 5) | 0.5 |
| Neo Dragocid Powder (Methyl parabene, | 0.2 |
| Sorbinic acid, Dehydro acetic acid, | |
| Propyl parabene) | |
| Coolant 4 | 2.0 |

TABLE F16
After Shave Tonic (quantities in % by weight)

| Components | INCI | Quantity |
|---|---|---|
| SymSol ® PF-3 | Water (Aqua), Pentylene | 3.00 |
| | Glycol, Sodium Lauryl | |
| | Sulfoacetate, Sodium Oleoyl Sarcosinate, | |
| | Sodium Chloride, Disodium Sulfoacetate, | |
| | Sodium Oleate, Sodium Sulfate | |
| SymSitive ® 1609 | Pentylene glycol, 4-t-butylcyclohexanol | 1.00 |
| Frescolat ® ML | Menthyl Lactate | 0.30 |
| Glycerol 99.5 P. | Glycerol | 5.00 |
| Water | Water (Aqua) | ad 100 |
| Extrapone ® Glacier | Glycerol, water (aqua) | 1.00 |
| Water GW | | |
| SymCalmin ® | Butylene Glycol, Pentylene Glycol, | 0.50 |
| | Hydroxyphenyl Propamidobenzoic Acid | |
| Dragosine ® | Carnosine | 0.10 |
| Hydrolite ® 5 | Pentylene Glycol | 5.00 |
| Ethanol 96 % | Alcohol Denat. | 5.00 |
| Color Pigment | Color Pigment | 0.05 |
| Hydrolite ® 5 | 1,2-Pentanediol | 0.15 |
| Coolant 2 | | 1.00 |

TABLE F17
Deodorant formulation as roll-on gel (amounts in % by weight)

| Ingredients (INCI) | Quantity |
|---|---|
| 1,3-Butylene glycol | 2.00 |
| PEG-40-hydrogenated castor oil | 2.00 |
| Hydroxyethyl cellulose | 0.50 |
| 1,2-Pentanediol (Hydrolite ® 5) | 0.30 |
| 1,3-Propanediol | 0.50 |
| 3-Phenylpropanol | 0.40 |
| Ethylhexyl glycerin | 0.10 |
| Coolant 1 | 0.50 |
| Water | ad 100 |

TABLE F18
Clear roll-on deodorant (quantities in % by weight)

| Components | INCI | Quantity |
|---|---|---|
| Methocel E4M Premium | Hydroxypropyl methylcellulose | 0.50 |
| Water | Water (Aqua) | ad 100 |
| Neo-PCL Water | Trideceth-9, PEG-5 | 1.00 |
| Soluble N | ethylhexanoate, water (aqua) | |
| Solubilizer | PEG-40 Hydrogenated | 3.00 |
| | Castor Oil, | |
| | Trideceth-9, Propylene | |
| | Glycol, Water (Aqua) | |
| Deolite | Dimethyl Phenylpropanol, | 0.50 |
| | Pentylene Glycol | |
| Locron LW | Aluminum Chlorohydrate | 25.00 |
| Aloe Vera Gel | Aloe Barbadensis Leaf Juice | 1.00 |
| Concentrate 10/1 | | |
| Propylene glycol | Propylene Glycol | 4.00 |
| −1.2 99 P GC | | |
| Ethanol 96 % | Alcohol Denat. | 30.00 |
| Hydrolite ® 5 | 1,2-Pentanediol | 1.00 |
| Coolant 4 | | 1.25 |

TABLE F19
Deodorant stick (quantities in % by weight)

| Ingredients (INCI) | Quantity |
|---|---|
| Sodium stearate | 8.00 |
| PPG-3 Myristyl ether | 70.00 |
| 1,2-Propylene glycol | 10.00 |
| 1,1-Dimethyl-3-phenylpropanol | 0.20 |
| 2-Butyloctanoic acid | 0.20 |
| 1,2-Pentanediol (Hydrolite ® 5) | 0.60 |
| Heptoxy-1,2-propanediol | 0.20 |
| Phenoxyethanol | 0.30 |
| Coolant 2 | 1.50 |
| Water | ad 100 |

TABLE F20
Antiperspirant stick (quantities in % by weight)

| Components | INCI | Quantity |
|---|---|---|
| PCL Liquid 100 | Cetearyl ethylhexanonate | ad 100 |
| Silicone Fluid 345 | Cyclomethicone | 10.00 |
| CRODACOL C90 | Cetyl Alcohol | 8.00 |
| SYNCROWAX HGLC | C18-36 triglycerides | 8.00 |
| CRODAMOL PTC | Pentaerythritol Tetracaprylate/Caprate | 5.00 |
| SYNCROWAX HRC | Tribehenin | 4.00 |
| VOLPO N5 | Oleth-5 | 1.00 |
| Titanium Dioxide | | 1.00 |
| Rezal 36GP | Aluminum Tetrachlorohydrex GLY | 20.00 |
| Dry Flo C | Aluminum Starch Octenyl Succinate | 22.50 |
| Hydrolite ® 5 | 1,2-Pentanediol | 0.60 |
| Preservative | Phenoxyethanol | 0.40 |
| Hexoxy-1,2-propanediol | | 0.10 |
| Coolant 1 | | 1.40 |

TABLE F21
Pump spray (quantities in % by weight)

| Components | INCI | Quantity |
|---|---|---|
| SymClariol ® | Decylene Glycol | 0.50 |
| Solubilizer | PEG-40 Hydrogenated Castor Oil, | 4.00 |
| | Trideceth-9, Propylene Glycol, Water | |
| | (Aqua) | |
| Neo-PCL Water | Trideceth-9, PEG-5 Ethylhexanoate, | 1.50 |
| Soluble N | Aqua | |
| SymRelief ® | Bisabolol. Zingiber Officinale | 0.10 |
| | (Ginger) Root Extract | |
| Water | Water (Aqua) | ad 100 |
| 1,2-Propylene Glycol | Propylene Glycol | 6.00 |
| Hydrolite ® 5 | 1,2-Pentanediol | 0.40 |
| SymDiol ® 68 | 1,2-hexanediol, caprylyl glycol | 0.20 |
| Coolant 1 | | 1.40 |

TABLE F22
Antiperspirant (quantities in % by weight)

| Ingredients (INCI) | Quantities |
|---|---|
| Reach AZP-908 SUF | 24.00 |
| Cyclomethicone (pentamer) | ad 100 |
| Polydecene (Silkflo 364 NF) | 17.50 |
| Neo Heliopan OS (ethylhexyl salicylate) | 2.50 |
| L-Menthyl lactate (Frescolate ML) | 0.25 |
| Polyethylene | 3.00 |
| Hydrogenated castor oil | 2.00 |
| Promyristyl PM-3 | 7.00 |
| PEG-8 distearate | 3.00 |
| Silicon dioxide (Cab-O-Sil M-5) | 1.00 |
| Stearyl alcohol | 15.00 |
| Octyldodecanol | 0.10 |
| 1,2-Pentanediol (Hydrolite ® 5) | 0.80 |
| 3-Phenylpropanol | 0.40 |
| Coolant 2 | 1.60 |

TABLE F23
Spray deodorant with triclosan (amounts in % by weight)

| Ingredients (INCI) | Quantity |
|---|---|
| PEG-40-hydrogenated castor oil | 3.00 |
| Ethylhexylglycerol (octoxyglycerol) | 0.80 |
| Ethanol | 40.00 |
| Citrate buffer | 0.50 |
| Triclosan ® (5-chloro-2-(2,4- | 0.25 |
| dichlorophenoxy)phenol) | |
| 1,2-Pentanediol (Hydrolite ® 5) | 0.75 |
| Phenoxyethanol | 0.40 |
| Coolant 3 | 1.40 |
| Water | ad 100 |

TABLE F24
O/W lotion (quantities in % by weight)

| Ingredients (INCI) | Quantity |
|---|---|
| Kerosene oil | 5.00 |
| Isopropyl palmitate | 5.00 |
| Cetyl alcohol | 2.00 |
| Beeswax | 2.00 |
| Ceteareth-20 | 2.00 |
| PEG-20-glyceryl stearate | 1.50 |
| Glycerol | 3.00 |
| 1,2-Pentanediol (Hydrolite ® 5) | 0.30 |
| Methylparaben | 0.30 |
| Coolant 4 | 1.00 |
| Water | ad 100.00 |

TABLE F25
Body lotion (quantities in % by weight)

| Ingredients (INCI) | Quantity |
|---|---|
| Cetearyl Alcohol | 2.00 |
| Ethylhexyl Isononanoate | 5.00 |
| Cetearyl Ethylhexanoate, Isopropyl Myristate | 3.00 |
| Glyceryl Oleate Citrate, Caprylic/Capric Triglyceride | 4.00 |
| Water (Aqua) | ad 100 |
| Carbomer | 0.30 |
| Sodium Benzoate | 0.100 |
| Propylene Glycol | 5.00 |
| Sodium hydroxide 30% solution | 0.30 |
| 1,2-Pentanediol (Hydrolite ® 5) | 0.30 |
| Triethylene Glycol, Imidazolidinyl Urea, | 0.30 |
| Methylparaben, Propylparaben, | |
| Dehydroacetic Acid | |
| Coolant 3 | 1.30 |

TABLE F26
Cream (quantities in % by weight)

| Ingredients (INCI) | Quantity |
|---|---|
| Kerosene oil | 10.00 |
| Ozokerite | 4.00 |
| Vaseline | 4.00 |
| Vegetable oil | 10.00 |
| Wool wax alcohol | 2.00 |
| Aluminum stearate | 0.40 |
| 1,2-Pentanediol (Hydrolite ® 5) | 2.00 |
| Phenoxyethanol | 0.50 |
| Coolant 2 | 1.50 |
| Water | ad 100 |

TABLE F27
Cream (quantities in % by weight)

| Components | INCI | Quantity |
|---|---|---|
| Dracorin ® CE | Glyceryl Stearate Citrate | 1.00 |
| Lanette ® O | Cetearyl Alcohol | 2.00 |
| Cutina ® GMS-V | Glyceryl stearate | 1.00 |
| Tegosoft ® MM | Myristyl myristate | 1.00 |
| Xiameter ® PMX-0246 | Cyclohexasiloxane (and) | 0.50 |
| Cyclosiloxane | Cyclopentasiloxane | |
| Dragoxat ® 89 | Ethylhexyl Isononanoate | 2.00 |
| PCL-Liquid 100 | Cetearyl ethylhexanoate | 4.00 |
| Neutral Oil | Caprylic/Capric Triglyceride | 4.00 |
| Carbopol ® Ultrez 21 | Acrylates/C10-30 Alkyl Acrylate | 0.20 |
| | Crosspolymer | |
| Keltrol ® CG-T | Xanthan Gum | 0.10 |
| Water | Water (Aqua) | ad 100 |
| Glycerol 99.5 P. | Glycerol | 3.00 |
| Propylene glycol | Propylene Glycol | 2.00 |
| −1.2 99 P GC | | |
| Sodium Benzoate | Sodium Benzoate | 0.10 |
| Sodium hydroxide | Sodium hydroxide | 0.50 |
| 10% solution | | |
| Hydrolite ® 5 | 1,2-Pentanediol | 0.30 |
| Euxyl ® K702 | Dehydroacetic Acid, Benzoic Acid, | 0.30 |
| | Phenoxyethanol, Polyaminopropyl | |
| | Biguanide, Ethylhexylglycerin | |
| Coolant 1 | | 2.00 |

TABLE F28
Hand and body cream (amounts in % by weight)

| Components | INCI | Quantity |
|---|---|---|
| Dracorin ® GOC | Glyceryl Oleate Citrate, | 2.00 |
| | Caprylic/Capric | |
| | Triglyceride | |
| PCL-Solid | Stearyl Heptanoate, | 2.50 |
| | Stearyl Caprylate | |
| Lanette ® O | Cetearyl Alcohol | 1.50 |
| Cutina ® GMS-V | Glyceryl stearate | 1.00 |
| Dragoxat ® 89 | Ethylhexyl Isononanoate | 3.00 |
| PCL-Liquid 100 | Cetearyl ethylhexanoate | 7.00 |
| Isodragol ® | Triisononanoin | 4.00 |
| Xiameter ® PMX-0345 | Cyclopentasiloxane (and) | 0.50 |
| Cyclosiloxane | Cyclohexasiloxane | |
| Water | Water (Aqua) | ad 100 |
| Carbopol ® Ultrez 21 | Acrylates/C10-30 Alkyl Acrylate | 0.20 |
| | Crosspolymer | |
| Keltrol ® CG-RD | Xanthan Gum | 0.10 |
| Glycerol 85 P. | Glycerol | 3.00 |
| DragoBetaGlucan | Water (Aqua), Butylene | 1.50 |
| | Glycol, Glycerol, | |
| | Avena Sativa (Oat) Kernel Extract | |
| Potassium sorbate | Potassium Sorbate | 0.10 |
| Sodium hydroxide | Sodium hydroxide | 0.50 |
| 10% solution | | |
| Hydrolite ® 5 | 1,2-Pentanediol | 0.20 |
| Euxyl ® K300 | Methyl-, butyl-, ethyl-, propyl-, | 0.30 |
| | isobutylparaben, phenoxyethanol | |
| Coolant 4 | | 1.00 |

TABLE F29
Face cream (amounts in % by weight)

| Components | INCI | Quantity |
|---|---|---|
| Emulsiphos ® | Potassium Cetyl Phosphate, | 1.50 |
| | Hydrogenated Palm Glycerides | |
| Cutina ® GMS-V | Glyceryl stearate | 1.70 |
| Lanette ® O | Cetearyl Alcohol | 3.00 |
| Tegosoft ® MM | Myristyl myristate | 1.00 |
| PCL-Liquid 100 | Cetearyl ethylhexanoate | 1.00 |
| Isodragol ® | Triisononanoin | 3.00 |
| Dragoxat ® 89 | Ethylhexyl Isononanoate | 4.00 |
| Avocado Oil | Persea Gratissima (Avocado) Oil | 3.00 |
| Abil ® 350 | Dimethicone | 0.50 |
| Covi-ox ® T-70 | Tocopherol | 0.10 |
| Edeta ® BD | Disodium EDTA | 0.10 |
| Carbopol ® Ultrez 21 | Acrylates/C10-30 Alkyl Acrylate | 0.30 |
| | Crosspolymer | |
| Keltrol ® CG-RD | Xanthan Gum | 0.150 |
| Water | Water (Aqua) | ad 100 |
| Glycerol 99.5 P. | Glycerol | 4.00 |
| Propylene glycol | Propylene Glycol | 3.00 |
| −1.2 99 P GC | | |
| SymMatrix ® | Maltodextrin. Rubus Fruticosus | 0.50 |
| | (Blackberry) Leaf Extract | |
| Sodium hydroxide | Sodium hydroxide | 0.50 |
| 10% solution | | |
| Hydrolite ® 5 | 1,2-Pentanediol | 0.30 |
| Euxyl ® K712 | Sodium Benzoate, | 0.20 |
| | Potassium Sorbate | |
| Coolant 3 | | 1.00 |

TABLE F30
Moisturizing cream (quantities in % by weight)

| Ingredients (INCI) | Quantity |
|---|---|
| PEG-7 hydrogenated castor oil | 6.00 |
| Cetearyl ethyl hexanoate | 10.00 |
| Isopropyl myristate | 5.00 |
| Mineral oil | 7.00 |
| Shea butter (Butyrospermum parkii) | 0.50 |
| Aluminum stearate | 0.50 |
| Magnesium stearate | 0.50 |
| Bisabolol | 0.20 |
| Quaternium-18-hectorite | 0.70 |
| Dipropylene glycol | 5.0 |
| Magnesium sulfate | 0.70 |
| 1,2-Pentanediol (Hydrolite ® 5) | 4.00 |
| Preservative (phenoxyethanol) | 0.20 |
| Coolant 1 | 1.00 |
| Aqua dem. | ad 100 |

TABLE F31
Anti-wrinkle cream (quantities in % by weight)

| Ingredients (INCI) | Quantity |
|---|---|
| Glyceryl Stearate Citrate | 1.00 |
| Glyceryl laurates | 1.00 |
| Cetearyl Alcohol (and) Myristyl Myristate | 3.00 |
| Cetearyl ethylhexanoate | 4.00 |
| Mineral oil | 4.00 |
| Cyclohexasiloxanes | 0.50 |
| Acrylates/C10-30 Alkyl Acrylate Crosspolymer | 0.20 |
| Water | ad 100 |
| 1,2-Hexanediol | 2.00 |
| Sodium hydroxide 10 % solution | 0.10 |
| Narcissus Tazetta Bulb Extract | 1.00 |
| 1,2-Pentanediol (Hydrolite ® 5) | 0.30 |
| Preservative (phenoxyethanol) | 0.50 |
| Coolant 1 | 1.50 |

TABLE F32
Disinfectant skin care oil (quantities in % by weight)

| Components | INCI | Quantity |
|---|---|---|
| Neutral Oil | Caprylic/Capric Triglyceride | ad 100 |
| Sweet Almond Oil | Prunus Dulcis | 20.00 |
| Dragoxat ® 89 | Ethylhexyl Isononanoate | 4.00 |
| Isopropyl Palmitate | Isopropyl Palmitate | 6.00 |
| PCL-Liquid 100 | Cetearyl ethylhexanoate | 15.00 |
| Dragosantol ® 100 | Bisabolol | 0.20 |
| Retinyl Acetate In | Retinyl Acetate | 0.50 |
| Oil (1 Mio le/G) | | |
| Vitamin E Acetate | Tocopheryl Acetate | 0.50 |
| Covi-ox ® T-70 | Tocopherol | 0.10 |
| Hydrolite ® 5 | 1,2-Pentanediol | 0.30 |
| Preservative | Methyl-, butyl-, ethyl-, propylparaben | 0.30 |
| Coolant 2 | | 1.00 |

TABLE F33
Antiseptic wound cream (quantities in % by weight)

| Ingredients (INCI) | Quantity |
|---|---|
| Sorbitan Isostearate, Hydrogenated | 6.00 |
| Castor Oil, Ceresin, Beeswax | |
| (Cera Alba) | |
| Petrolatum | 21.00 |
| Cera Alba | 5.00 |
| Cetearyl Alcohol | 7.00 |
| Prunus Dulcis | 7.00 |
| Lanolin | 5.00 |
| Paraffinum liquidum | 12.00 |
| Water (Aqua) | ad 100 |
| Panthenol | 7.00 |
| Magnesium sulfates | 0.70 |
| 1,2-Pentanediol (Hydrolite ® 5) | 1.00 |
| Tocopheryl Acetate | 1.00 |
| Octenidine dihydrochloride | 0.10 |
| Phenoxyethanol | 0.50 |
| Coolant 3 | 1.50 |

TABLE F34
Moisture mask (quantities in % by weight)

| Components | INCI | Quantity |
|---|---|---|
| Water | Water (Aqua) | ad 100 |
| Stabileze QM | PVM/MA Decadiene Cross- | 0.50 |
| | polymer | |
| Biotive ® L-Arginine | Arginine | 0.75 |
| Actipone ® Laminaria | Glycerol, water (aqua), Laminaria | 1.00 |
| Saccharina GW | Saccharina Extract | |
| Extrapone ® Cucumber | Water (Aqua), Propylene Glycol, | 1.00 |
| | Cucumis Sativus (Cucumber) Juice | |
| Glycerol 99.5 P. | Glycerol | 7.00 |
| Neo Actipone ® Soap | Sapindus Mukurossi Peel Extract | 0.50 |
| Nutshell | | |
| Color I | Color | 0.01 |
| Hydrolite ® 5 | Pentylene Glycol | 5.00 |
| Solubilizer | PEG-40 Hydrogenated Castor Oil, | 0.60 |
| | Trideceth-9, Water (Aqua) | |
| Hydrolite ® 5 | 1,2-Pentanediol | 0.08 |
| Preservative | Phenoxyethanol | 0.40 |
| Coolant 4 | | 1.00 |

TABLE F35
Sprayable disinfectant gel (quantities in % by weight)

| Components | INCI | Quantity |
|---|---|---|
| Water | Water (Aqua) | ad 100 |
| Stabileze QM | PVM/MA Decadiene Cross- | 0.25 |
| | polymer | |
| Sodium hydroxide 10% | Sodium hydroxide | 0.40 |
| solution | | |
| Caffeine pure | Caffeine | 0.50 |
| Extrapone ® Horse | Propylene Glycol, Water (Aqua), | 1.00 |
| Chestnut | Glucose, Aesculus Hippocastanum | |
| | (Horse Chestnut) Seed Extract, | |
| | Lactic Acid | |
| Hydrolite ® 5 | 1,2-Pentanediol | 3.00 |
| 1,3-Butylene Glycol | Butylene Glycol | 5.00 |
| Biotive ® Esculin | Esculin | 0.30 |
| Sesquihydrate | | |
| Ethanol 96% | Alcohol Denat. | 10.00 |
| Solubilizer | PEG-40 Hydrogenated Castor Oil, | 0.50 |
| | Trideceth-9, Water (Aqua) | |
| Octenidine | | 0.10 |
| dihydrochloride | | |
| Preservative | Phenoxyethanol | 0.70 |
| Coolant 2 | | 1.00 |

TABLE F36
Mineral washing and cleaning gel (quantities in % by weight)

| Components | INCI | Quantity |
|---|---|---|
| Water | Water (Aqua) | ad 100 |
| Pionier ® NP 37 G | Sodium carbomer | 1.50 |
| SymSol ® PF-3 | Water (Aqua), Pentylene Glycol, | 5.00 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Hydroviton ® 24 | Water (Aqua), Pentylene Glycol, | 1.00 |
| | Glycerol, Sodium Lactate, Lactic | |
| | Acid, Serine, Urea, Sorbitol, | |
| | Sodium Chloride, Allantoin | |
| Extrapone ® Silk GW | Water (Aqua), Glycerol, | 1.00 |
| | Hydrolyzed Silk | |
| Hydrolite ® 5 Green | Hydrolite ® 5 Green | 4.00 |
| Actipearls Red Star | Water (Aqua), Propylene Glycol, | 1.00 |
| # DH10402/6 | Algin, Gellan Gum, Xanthan Gum, | |
| | Calcium Chloride, Cl 12490 | |
| | (Pigment Red 5), Mica (CI 77019), | |
| | Titanium Dioxide (CI 77891) | |
| 1,2-Pentanediol | 1,2-Pentanediol | 0.50 |
| (Hydrolite ® 5) | | |
| 3-Phenylpropanol | | 0.70 |
| Coolant 4 | | 0.30 |

TABLE F37
Anti-acne wash (quantities in % by weight)

| Ingredients (INCI) | Quantity |
|---|---|
| Water (Aqua) | ad 100 |
| Polyquaternium-7 | 0.50 |
| Cocamidopropyl betaine 9,000 | 9.00 |
| Coco Glucoside 2,000 | 2.00 |
| Polysorbate 80, Glycerol, | 1.00 |
| Gossypium Herbaceum, (Cotton) Seed Oil, | |
| Water (Aqua) | |
| Trideceth-9, PEG-5 ethylhexanoate, water (aqua) | 1.00 |
| Glycereth-90 Isostearate, Laureth-2 | 0.50 |
| Sodium Laureth Sulfate 37,000 | 37.00 |
| Glycerol, Triticum Vulgare (Wheat) Gluten, | 1.00 |
| Water (Aqua) | |
| Sodium Chloride | 0.30 |
| 1,2-Pentanediol (Hydrolite ® 5) | 1.00 |
| Phenoxyethanol, methylparaben, ethylparaben, | 0.30 |
| butylparaben, propylparaben, isobutylparaben | |
| Coolant 5 | 1.50 |

TABLE F38
Sunscreen formulation (quantities in % by weight)

| Ingredients (INCI) | Quantity |
|---|---|
| Ethylhexyl cinnamic acid | 7.50 |
| Benzophenone-3 | 2.00 |
| Polyglyceryl dimer soyate | 0.80 |
| Sorbitane stearate | 1.00 |
| Tocopheryl acetate | 0.50 |
| Glyceryl stearate, PEG-100 stearate | 3.00 |
| PEG-40, hydrogenated castor oil | 1.00 |
| Titanium dioxide, Aluminum oxide hydrate, | 3.00 |
| Dimethicone/Methicone Copolymer | |
| Butyrospermum parkii (shea butter) | 1.00 |
| C12-15 alkyl benzoate | 6.50 |
| Butylene glycol | 5.00 |
| Xanthan gum | 0.30 |
| Disodium EDTA | 0.10 |
| Allantoin | 0.10 |
| Polyacryl amide, C13-14 isoparaffin, Laureth-7 | 1.00 |
| 1,2-Pentanediol (Hydrolite ® 5) | 5.00 |
| 4-t butylcyclohexanol | 1.00 |
| PreservativePreservatives (methyl-, butyl-, | 0.30 |
| ethyl-, propylparaben, phenoxyethanol) | |
| Coolant 2 | 2.00 |
| Aqua dem. | ad 100 |

TABLE F39
Sunscreen spray (quantities in % by weight)

| Components | INCI | Quantity |
|---|---|---|
| Water, demineralized. | Water (aqua) | ad 100 |
| Glycerol | Glycerol | 4.00 |
| 1,3-Butylene glycol | Butylene glycol | 5.00 |
| D-Panthenol | Panthenol | 0.50 |
| Lara Care A-200 | Galactoarabinan | 0.25 |
| Baysilone oil M 10 | Dimethicone | 1.00 |
| Edeta BD | Disodium EDTA | 0.10 |
| Copherol 1250 | Tocopheryl acetate | 0.50 |
| Cetiol OE | Dicaprylyl ether | 3.00 |
| Neo Heliopan ® HMS | Homosalads | 5.00 |
| Neo Heliopan ® AV | Ethylhexyl methoxycinnamate | 6.00 |
| Neo Heliopan ® 357 | Butyl methoxydibenzoylmethane | 1.00 |
| Corapan TQ | Diethylhexylnaphthalates | 2.00 |
| Alpha bisabolol | Bisabolol | 0.10 |
| Pemulen TR-2 | Acrylates/C10-30 alkyl acrylate | 0.25 |
| | crosspolymer | |
| NaOH, 10% | Sodium hydroxide | 0.60 |
| Hydrolite ® 5 | 1,2-Pentanediol | 0.20 |
| Phenoxyethanol | Phenoxyethanol | 0.40 |
| Solbrol M | Methylparaben | 0.10 |
| Solbrol P | Propylparaben | 0.10 |
| Coolant 3 | | 1.00 |

TABLE F40
Sun protection spray O/W, SPE 15-20 (quantities in % by weight)

| Components | INCI | Quantity |
|---|---|---|
| Dracorin ® GOC | Glyceryl Oleate Citrate, | 2.00 |
| | Caprylic/Capric Triglyceride | |
| Corapan ® TQ | Diethylhexyl 2,6-naphthalates | 3.00 |
| Neo Heliopan ® HMS | Homosalads | 7.00 |
| Neo Heliopan ® OS | Ethylhexyl salicylate | 5.00 |
| Neo Heliopan ® 357 | Butyl methoxydibenzoylmethane | 3.00 |
| Isoadipate | Diisopropyl adipate | 6.00 |
| Baysilone ® Oil M10 | Dimethicone | 1.00 |
| Edeta ® BD | Disodium EDTA | 0.10 |
| Vitamin E Acetate | Tocopheryl Acetate | 0.50 |
| Dragosantol ® 100 | Bisabolol | 0.10 |
| Pemulen ® TR-2 | Acrylates/C10-30 Alkyl Acrylate | 0.25 |
| | Crosspolymer | |
| Water | Water (Aqua) | ad 100 |
| Glycerol 99.5 P. | Glycerol | 4.00 |
| Butylene Glycol | Butylene Glycol | 5.00 |
| Neo Heliopan ® Hydro | Phenylbenzimidazole Sulfonic | 8.00 |
| (103089), used as | Acid | |
| 25% aq. solution | | |
| Biotive ® L-Arginine | Arginine | 0.55 |
| Hydrolite ® 5 | 1,2-Pentanediol | 0.40 |
| Sobrol M | Methylparaben | 0.30 |
| Coolant 1 | | 1.00 |

TABLE F41
Sunscreen soft cream (W/O), SPF 40 (quantities in % by weight)

| Components | INCI | Quantity |
|---|---|---|
| Dehymuls PGPH | Polyglyceryl-2- | 5.00 |
| | dipolyhydroxystearate | |
| Copherol 1250 | Tocopheryl acetate | 0.50 |
| Permulgin 3220 | Ozocerite | 0.50 |
| Zinc stearate | Zinc stearate | 0.50 |
| Tegosoft TN | C12-15 alkyl benzoate | 10.00 |
| Neo Heliopan ® E1000 | Isoamyl-p-methoxycinnamate | 2.00 |
| Neo Heliopan ® 303 | Octocrylene | 5.00 |
| Neo Heliopan ® MBC | 4-Methylbenzylidene camphor | 3.00 |
| Zinc oxide, neutral | Zinc oxide | 5.00 |
| Distilled water | Water (aqua) | ad 100 |
| EDETA BD | Disodium EDTA | 0.10 |
| Glycerol | Glycerol | 4.00 |
| Magnesium sulfate | Magnesium sulfate | 0.50 |
| Hydrolite ® 5 | 1,2-pentanediol | 0.30 |
| Symdiol ® 68 | 1,2-hexanediol, | 0.30 |
| | caprylyl glycol | |
| Coolant 1 | | 1.00 |

TABLE F42
Sunscreen milk (W/O) (quantities in % by weight)

| Components | INCI | Quantity |
|---|---|---|
| Dehymuls PGPH | Polyglyceryl-2 | 3.00 |
| | dipolyhydroxystearate | |
| Beeswax 8100 | Beeswax | 1.00 |
| Monomuls 90-0-18 | Glyceryl oleate | 1.00 |
| Zinc stearate | Zinc stearate | 1.00 |
| Cetiol SN | Cetearyl isononanoate | 5.00 |
| Cetiol OE | Dicaprylyl ether | 5.00 |
| Tegosoft TN | C12-15 alkyl benzoate | 4.00 |
| Vitamin E | Tocopherol | 0.50 |
| Neo Heliopan ® OS | Ethylhexyl salicylate | 5.00 |
| Neo Heliopan ® AV | Ethylhexyl methoxycinnamate | 7.50 |
| Uvinul ® T150 | Ethylhexyl triazone | 1.50 |
| Water, distilled | Water (Aqua) | ad 100 |
| Trilon BD | Disodium EDTA | 0.10 |
| Glycerol | Glycerol | 5.00 |
| Neo Heliopan ® AP | Disodium phenyl | 15.00 |
| 10% solution. | dibenzimidazole | |
| neutralized with NaOH | tetrasulfonate | |
| Hydrolite ® 5 | 1,2-Pentanediol | 0.25 |
| Alpha bisabolol | Bisabolol | 0.10 |
| SymOcide ® PT | Phenoxyethanol, Tropolone | 0.25 |
| Coolant 2 | | 1.00 |

TABLE F43
After sun gel (quantities in % by weight)

| Components | INCI | Quantity |
|---|---|---|
| SymSol ® PF-3 | Water (Aqua), Pentylene Glycol, | 3.000 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Glycerol 99.5 P. | Glycerol | 5.000 |
| SymHelios ® 1031 | Benzylidene | 0.100 |
| | dimethoxydimethyline danone | |
| Water | Water (Aqua) | ad 100 |
| Pemulen ® TR-2 | Acrylates/C10-30 Alkyl | 1.000 |
| | Acrylate Crosspolymer | |
| D-Panthenol 75 W | Panthenol | 0.500 |
| SymFinity ® 1298 | Echinacea Purpurea Extract | 0.100 |
| Extrapone ® Pearl | Water (aqua), glycerol, | 1.000 |
| GW | hydrolyzed pearl, xanthan | |
| | gum | |
| Sodium hydroxide | Sodium hydroxide | 2.500 |
| 10% solution | | |
| Ethanol 96% | Alcohol Denat. | 15.000 |
| Hydrolite ® 5 | 1,2-Pentanediol | 0.20 |
| SymOcide ® PS | Phenoxyethanol, 1,2-hexanediol, | 0.50 |
| | decyleneglycol | |
| Coolant 3 | | 1.00 |

TABLE F44
After Sun Lotion (quantities in % by weight)

| Ingredients (INCI) | Quantity |
|---|---|
| Acrylates/C10-30 alkyl acrylate | 0.4 |
| crosspolymer | |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| 1,2-Hexanediol | 0.60 |
| Coolant 4 | 1.50 |
| 1,2-Pentanediol (Hydrolite ® 5) | 4.0 |
| Aqua dem. | ad 100 |
| Triethanolamines | 0.2 |

TABLE F45
Hair styling gel (quantities in % by weight)

| Ingredients (INCI) | Quantity |
|---|---|
| Water | ad 100 |
| PVM/MA Decadiene Crosspolymer | 0.60 |
| PVP | 3.00 |
| Isocetyl stearate | 4.00 |
| Ethylhexyl methoxycinnamate | 0.50 |
| Aminomethyl propanol | 0.40 |
| 1,2-Pentanediol (Hydrolite ® 5) | 0.60 |
| SymDiol ® 68T (1,2-hexanediol, | 0.30 |
| 1,2-octanediol, Tropolone) | |
| Phenoxyethanol | 0.20 |
| Coolant 5 | 1.50 |

TABLE F46
Silicone emulsion (quantities in % by weight)

| Components | Quantity |
|---|---|
| Potassium Cetyl Phosphate, | 1.00 |
| Hydrogenated Palm Glycerides | |
| Cyclohexasiloxanes | 4.00 |
| Cetearyl Alcohol | 1.50 |
| Phenyl Trimethicone | 3.00 |
| Stearyl Heptanoate, Stearyl Caprylate | 3.00 |
| Dimethicone | 1.00 |
| Xanthan Gum | 0.20 |
| Isoamyl p-methoxycinnamate | 5.00 |
| Water | ad 100 |
| Methylpropanediol | 3.00 |
| 1,2-Pentanediol (Hydrolite ® 5) | 0.30 |
| Diazolidinyl urea | 0.10 |
| Coolant 2 | 1.50 |

TABLE F47
Microemulsion gel (quantities in % by weight)

| Ingredients (INCI) | Quantity |
|---|---|
| Glycerol isostearate | 1.80 |
| Octoxyglycerol | 1.00 |
| Ceteareth-15 | 5.20 |
| PEG-150 Distearate | 1.00 |
| Aluminum chlorohydrate | 5.00 |
| Isotridecyl isononanoate | 3.30 |
| Cyclomethicone | 6.60 |
| 1,2-Pentanediol (Hydrolite ® 5) | 0.70 |
| Euxyl ® K145 (Methylchloroisothiazolinone, | 0.10 |
| Methylisothiazlinone, Bronopol) | |
| Coolant 3 | 1.00 |
| Water | ad 100 |

TABLE F48
Air freshener in gel form (quantities in % by weight)

| Ingredients (INCI) | Quantity |
|---|---|
| Demineralized water | ad 100 |
| Genugel ® X-6424 (carrageenan) | 2.00 |
| Arkopal ® N 100 or Tergitol ® NP 10 (emulsifier) | 3.50 |
| 1,2-Pentanediol (Hydrolite ® 5) | 0.60 |
| Preventol ® D 7 (5-chloro-2-methyl-4- | 0.10 |
| isothiazolin-3-one, 2-methyl-2H-isothiazol-3-one) | |
| Coolant 2 | 1.00 |

TABLE F49
Textile cleaners (quantities in % by weight)

| Ingredients (INCI) | Quantity |
|---|---|
| Aqua | ad 100 |
| Dialkyl ester ammomium methosulfate | 16.60 |
| Polydimethylsiloxanes | 0.30 |
| Magnesium chlorides | 10.00 |
| 1,2-Pentanediol (Hydrolite ® 5) | 0.60 |
| Mixture of 5-Chloro-2-methyl-2H- | 0.10 |
| isothiazol-3-one and 2-Methyl-2H- | |
| isothiazol-3-one | |
| Coolant 3 (encapsulated) | 0.40 |

TABLE F50
Toothpaste (quantities in % by weight)

| Ingredients (INCI) | Quantity |
|---|---|
| Deionized water | ad 100 |
| Sorbitol 70% | 45.00 |
| Solbrol ® M Na salt (p-hydroxybenzoic | 0.15 |
| acid alkyl ester) | |
| Trisodium phosphate | 0.10 |
| Saccharin, 450-fold | 0.20 |
| Sodium monofluorophosphate | 1.12 |
| Polyethylene glycol PEG 1500 | 5.00 |
| Sident 9 (abrasive silicon dioxide) | 10.00 |
| Sident 22 S (thickening silicon dioxide) | 8.00 |
| Sodium carboxymethylcellulose | 0.90 |
| Titanium dioxide | 0.50 |
| Sodium lauryl sulfate (SLS) | 1.50 |
| Coolant 4 | 0.01 |

TABLE F51
Mouthwash (quantities in % by weight)

| Ingredients (INCI) | Quantity |
|---|---|
| Ethanol | 10.00 |
| Cremophor ® CO 40 (BASF, detergent) | 1.00 |
| Benzoic acid | 0.12 |
| Peppermint-lemon balm flavor, containing | 0.25 |
| 0.4% by weight pellitorin and 10% by | |
| weight coolant 4 | |
| Demineralized water | ad 100 |
| Sorbitol, 70% | 5.00 |
| Sodium saccharin 450 | 0.07 |
| L-Blue 5000 e.c., 1% in water (colorant) | 0.10 |

TABLE F52
Toothpaste against plaque with efficacy against
bad breath (amounts in % by weight)

| Ingredients (INCI) | Quantity |
|---|---|
| Na-carboxymethylcellulose | 1.00 |
| Glycerine | 12.50 |
| Sorbitol 70%, in water | 29.00 |
| Na-saccharinate | 0.20 |
| Na-fluoride | 0.22 |
| Azacycloheptane-2,2-diphosphoic | 1.00 |
| acid, di-sodium salt | |
| Bromochlorophene | 0.10 |
| Peppermint flavor | 0.90 |
| Coolant 1 | 0.10 |
| Parsley seed oil | 0.15 |
| Abrasive silica | 15.00 |
| Thickening silica | 5.00 |
| Sodium dodecyl sulfate (SDS) | 1.50 |
| Water dist. | ad 100.00 |

TABLE F53
Toothpaste and mouthwash as a 2-in-1
product (quantities in % by weight)

| Ingredients (INCI) | Quantity |
|---|---|
| Sorbitol | 40.00 |
| Glycerine | 20.00 |
| Ethanol | 5.00 |
| Water | ad 100.00 |
| Solbrol M, Na salt (methylparaben, Na salt) | 0.15 |
| Na monofluorophosphate | 0.75 |
| Saccharin | 0.20 |
| Sident 9 (abrasive silicon dioxide) | 20.00 |
| Sident 22 S (thickening silicon dioxide) | 2.00 |
| Sodium carboxymethylcellulose | 0.30 |
| Sodium lauryl sulfate (SDS) | 1.20 |
| Color (1% in water) | 0.50 |
| Coolant 1 | 1.00 |

TABLE F54
Other toothpaste formulation (amounts in % by weight)

| Ingredient | Quantity |
|---|---|
| Demineralized water | ad 100 |
| Sorbitol (70%) | 45.00 |
| Solbrol ® M, sodium salt | 0.15 |
| (Bayer AG, p-hydroxybenzoic acid alkyl ester) | |
| Trisodium phosphate | 0.10 |
| Saccharin, 450 times | 0.20 |
| Sodium monofluorophosphate | 1.12 |
| Polyethylene glycol 1500 | 5.00 |
| Sident 9 (abrasive silicon dioxide) | 10.00 |
| Sident 22 S (thickening silicon dioxide) | 8.00 |
| Sodium carboxymethylcellulose | 0.90 |
| Titanium dioxide | 0.50 |
| Demineralized water | 4.53 |
| Sodium lauryl sulfate | 1.50 |
| Coolant 1 | 1.00 |

### Formulation Examples for Food Preparations

The following formulation examples F55 to F63 show a wide variety of formulations for food preparations. Coolant 1 here means compound B-11 according to the invention, coolant 2 means compound A-02, coolant 3 means compound A-09, coolant 4 means compound A-10. The coolants were used in pure form.

TABLE F55
Chewing gum (quantities in % by weight)

| Ingredients (INCI) | Quantity |
|---|---|
| Chewing gum base (“gum-base”) | 15-25 |
| Glucose syrup | 20-30 |
| Powdered sugar | 50-60 |
| Coolant 1 | 0.001-10 |
| Plasticizer (e.g. glycerine) | 1-2 |
| Water | 3-6 |

TABLE F56
Other chewing gum formulations (amounts in % by weight)

| Ingredients (INCI) | Quantity I | Quantity II | Quantity III |
|---|---|---|---|
| Chewing gum base K2 (28.5% terpene resin, | 25.30 | 27.30 | 26.30 |
| 33.9% polyvinyl acetate (MW = 14000), | | | |
| 16.25% hydrogenated vegetable oil, | | | |
| 5.5% mono- and diglycerides, | | | |
| 0.5% polyisobutene (MW 75000), | | | |
| 2.0% butyl rubber (isobutene-isoprene copolymer), | | | |
| 4.6% amorphous silicon dioxide | | | |
| (water content approx. 2.5%), | | | |
| 0.05% antioxidant tert.butylhydroxytoluene (BHT), | | | |
| 0.2% lecithin, and 8.5% calcium carbonate) | | | |
| Sorbitol | 61.48 | 59.48 | 61.48 |
| Glycerine | 2.40 | 2.40 | 2.40 |
| Lecithin | 7.00 | 7.00 | 7.00 |
| Aspartame | 0.14 | 0.14 | 0.14 |
| Encapsulated aspartame | 0.68 | 0.68 | 0.68 |
| Menthol, spray-dried | 0.50 | — | |
| Cherry flavor, spray-dried | — | 1.20 | |
| Coolant 1 | 2.50 | 1.80 | 1.68 |

TABLE F57
Sugar-free chewing gum (quantities in % by weight)

| Ingredients (INCI) | Quantity I | Quantity II |
|---|---|---|
| Chewing gum base, Company “Jagum T” | 30.00 | 30.00 |
| Sorbitol, powdered | 39.00 | 39.00 |
| Isomalt ® (Palatinit GmbH) | 9.50 | 9.50 |
| Xylitol | 2.00 | 2.00 |
| Mannitol | 3.00 | 3.00 |
| Aspartame ® | 0.10 | 0.10 |
| Acesulfame ® K | 0.10 | 0.10 |
| Emulgum ® (Colloides Naturels, Inc.) | 0.30 | 0.30 |
| Sorbitol, 70% | 14.00 | 14.00 |
| Glycerine | 1.00 | 1.00 |
| Spearmint-peppermint-eucalyptus flavor, | 1.00 | — |
| containing 5% by weight of coolant 2 | | |
| Coolant 2 | — | 1.20 |

TABLE F58
Pudding, recipe (for 100 ml) (quantities in grams)

| Ingredients (INCI) | Quantity |
|---|---|
| Fat-free dry milk | 10.715 |
| Sucrose | 5.000 |
| Novelose Starch, National Starch | 7.000 |
| Vegetable oil mixture | 2.200 |
| Carrageenan | 0.016 |
| Vanilla flavor | 0.500 |
| Sodium stearoyl-2-lactylate | 0.095 |
| Yellow dye | 0.189 |
| Magnesium phosphate | 0.165 |
| Vitamin premix | 1.840 |
| Trace element premix | 0.015 |
| Coolant 3 | 0.500 |
| Water | ad 100 |

TABLE 59
Chewy sweets (quantities in % by weight)

| Ingredients (INCI) | Quantity |
|---|---|
| Water | ad 100 |
| Sugar (refined sugar C4) | 42.10 |
| Glucose syrup (dextrose 40) | 37.30 |
| Hardened vegetable fat, | 6.60 |
| melting point 32-36° C. | |
| Lecithin (emulsifier, soy lecithin) | 0.30 |
| Gelatine (pork gelatine) | 0.80 |
| Fondant type-S30 | 4.80 |
| Raspberry flavor | 0.22 |
| Coolant 4 | 0.08 |

TABLE 60
Fruit gums (quantities in % by weight)

| Ingredients (INCI) | Quantity |
|---|---|
| Water | ad 100 |
| Sucrose | 34.50 |
| Glucose syrup, DE 40 | 31.89 |
| Iso Syrup C* Tru Sweet 0 1750 | 1.50 |
| (Cerestar GmbH) | |
| Gelatine 240 Bloom | 8.20 |
| Yellow and red dye | 0.01 |
| Citric acid | 0.20 |
| Coolant 1 | 0.10 |

TABLE 61
Gelatine capsule for direct consumption (quantities in % by weight)

| Ingredients (INCI) | Quantity |
|---|---|
| Gelatine shell: | |
| Glycerine | 2.014 |
| Gelatine 240 Bloom | 7.91 |
| Sucralose | 0.065 |
| Allura red | 0.006 |
| Brilliant blue | 0.005 |
| Core composition: | |
| Vegetable oil triglyceride | 79.39 |
| (coconut oil fraction) | |
| Cinnamon aniseed flavor | 10.00 |
| Neotame and aspartame | 0.01 |
| Sucralose | 0.22 |
| Coolant 2 | 0.33 |
| Vanillin | 0.05 |

TABLE 62
Non-stick chewing gum (quantities in % by weight)

| Ingredients (INCI) | Quantity I | Quantity II | Quantity III |
|---|---|---|---|
| Chewing gum base K1 (2.0% butyl rubber | 26.00 | 26.00 | 26.00 |
| (isobutene-isoprene copolymer, MW 400000), | | | |
| 6.0% polyisobutene (MW = 43800), | | | |
| 43.5% polyvinyl acetate (MW = 12000), | | | |
| 31.5% polyvinyl acetate (MW = 47000), | | | |
| 6.75% triacetin and 10.25% calcium carbonate) | | | |
| Triacetin | 0.25 | 0.25 | 0.25 |
| Lecithin | 0.50 | 0.50 | 0.50 |
| Sorbitol, crystalline | ad 100 | ad 100 | ad 100 |
| Mannitol | 15.30 | 15.20 | 15.10 |
| Glycerine | 12.10 | 12.00 | 11.80 |
| Aspartame | 0.17 | 0.17 | 0.17 |
| Encapsulated aspartame | 1.08 | 1.08 | 1.08 |
| Amorphous silica | 1.00 | 1.00 | 1.00 |
| Cottonseed oil | 0.05 | 0.05 | 0.05 |
| Polyoxyethylene sorbitan | 1.00 | 1.00 | 1.00 |
| monolaurate (E-432) | | | |
| Encapsulated spearmint flavor | 0.20 | 0.10 | 0.20 |
| (contains I-carvone) | | | |
| Encapsulated wintergreen flavor | — | 0.20 | — |
| (contains methyl salicylate) | | | |
| Coolant 3 | 1.00 | 1.40 | 1.70 |

TABLE F63
Centre-filled hard candy (quantities in % by weight)

| Ingredients (INCI) | Quantity I | Quantity II |
|---|---|---|
| Mixture A (wrapper) (80% of the sweets) | | |
| Sugar (sucrose) | 58.12 | 49.39 |
| Glucose syrup (solids content 80%) | 41.51 | 49.37 |
| Coolant 1 | 0.17 | 0.25 |
| I-menthol | 0.10 | — |
| Lemon oil | 0.10 | 0.10 |
| Citric acid | — | 0.91 |
| Total: | 100 | 100 |
| Mixture B (core) (20% of the sweets) | | |
| High fructose corn syrup (solid sugar | 84.38 | 84.36 |
| content 85%, just under 15% water) | | |
| Glycerine | 15.00 | 15.00 |
| Lecithin | 0.02 | 0.02 |
| Cinnamon oil | — | 0.32 |
| Spearmint oil | 0.28 | — |
| Capsaicin | 0.05 | — |
| Vanillyl alcohol n-butyl ether | — | 0.10 |
| Red dye, as 5% aqueous solution | 0.20 | 0.20 |
| Vanillin | 0.07 | — |
| Total: | 100 | 100 |

1. Physiological coolant of the general formula (Va) 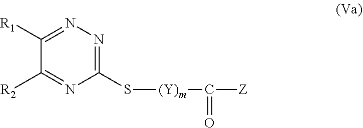
or the general formula (VIa) 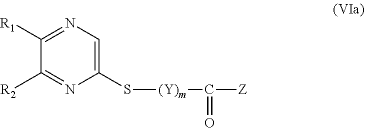
where in formulae (Va) and (VIa) in each case
the radicals R1 and R2 may be identical or different and have the following meanings independently of one another:
R1 H; or
a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group —R(C═O)—; or
an optionally substituted cycloalkyl group; or
an optionally substituted aryl group; or
an optionally substituted heterocycloalkyl group; or
is an optionally substituted heteroaryl group;
R2 H; or
a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is an optionally substituted heteroaryl group;
or R1 and R2 together with the C atoms to which they are bonded form a conjugated or non-conjugated ring system;
Y is an optionally substituted branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—; or
an optionally substituted alkylaryl group; or
is an optionally substituted alkylheteroaryl group;
Z in the general formula (Va) NH2; or
an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
Z in the general formula (VIa) NH₂; or
an NHRa group or an NRaRb group, with the exception of formula —NH-phenyl and —N(CH₃)-phenyl; or
an optionally substituted linear or branched alkyl group; or
an optionally substituted linear or branched alkenyl group; or
an optionally substituted linear or branched alkylthio group; or
an optionally substituted linear or branched alkoxy group, with the exception of —OC2H5 and —OC(CH₃)₃; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
m is 1;
whereby
the group Q is a radical selected from the group consisting of: halogen, —OA, —SA, —NBB, —CF₃, —CN, —OCN, —SCN, —NO, —NO₂, —S(O)₂^{a}, —S(O)₂OA, —OS(O)₂^{a}, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁-C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is in particular an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group,
is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
is in particular an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
Ra and/or Rb an optionally substituted linear or branched alkyl group, preferably a C₁- to C₃-alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
wherein the substituents are selected independently of one another;
and their salts, in particular acid addition salts, with inorganic or organic acids, whereby the coolants may be present in stereoisomerically pure form or as mixtures of different stereoisomers;
or the general formula (VIIa) 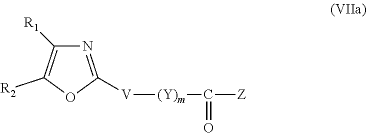
or the general formula (VIIIa) 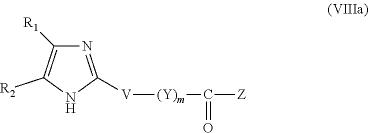
where in each of the formulae (VIIa) and (VIIIa)
the radicals R1 and R2 may be identical or different and have the following meanings independently of one another:
R1 H; or
a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group —R(C═O)—; or
an optionally substituted cycloalkyl group; or
an optionally substituted aryl group; or
an optionally substituted heterocycloalkyl group; or
is an optionally substituted heteroaryl group;
R2 H; or
a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is an optionally substituted heteroaryl group;
or R1 and R2 together with the C atoms to which they are bonded form a conjugated or non-conjugated ring system;
V S; or
SO₂; or
an optionally substituted linear or branched alkyl group; or
an optionally substituted cycloalkyl group; or
is piperidinyl;
Y is an optionally substituted branched alkyl group; or
an optionally substituted linear or branched alkenyl group; or
an optionally substituted linear or branched alkynyl group; or
an optionally substituted linear or branched alkoxy group; or
is an optionally substituted acyl group R—(C═O)—;
Z NH₂; or
an NHRa group; or
an NRaRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
is NH and together with the X forms a heterocyclic ring;
m is 1;
whereby
the group Q is a radical selected from the group consisting of: halogen, —OA, —SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁-C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is in particular an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group,
is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
is in particular an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
Ra and/or Rb an optionally substituted linear or branched alkyl group; preferably a C₁- to C₃-alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
wherein the substituents are selected independently of one another;
and their salts, in particular acid addition salts, with inorganic or organic acids, whereby the coolants may be present in stereoisomerically pure form or as mixtures of different stereoisomers.

2. Physiological coolant according to claim 1, wherein in the general formulae (Va) to (VIIIa) Y stands for a substituted methylene group, preferably for a methylene group which is substituted by a methyl group, an ethyl group, a linear or branched butyl group or a linear or branched propyl group.

3. Physiological coolant according to claim 1, wherein the physiological coolant of the general formulae (Va) or (Via) is selected from the group consisting of the compounds according to Table A below:
TABLE A

| Compound | Structure | IUPAC name |
|---|---|---|
| B-01 | 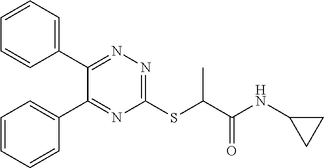 [SMILES: CC(Sc1nnc(-c2ccccc2)c(-c2ccccc2)n1)C(=O)NC1CC1] | N-Cyclopropyl-2-[(5,6-diphenyl- 1,2,4-triazin-3- yl)sulfanyl]propanamide |
| B-02 | 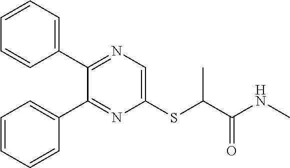 [SMILES: CNC(=O)C(C)Sc1cnc(-c2ccccc2)c(-c2ccccc2)n1] | 2-(5,6-diphenylpyrazin-2- yl)sulfanyl-N-methyl-propanamide |
| B-03 | 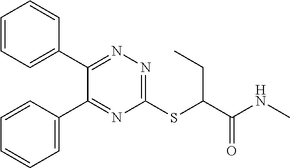 [SMILES: CCC(Sc1nnc(-c2ccccc2)c(-c2ccccc2)n1)C(=O)NC] | 2-[(5,6-diphenyl-1,2,4-triazin-3- yl)sulfanyl]-N-methyl-butanamide |
| B-05 | 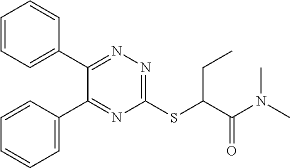 [SMILES: CCC(Sc1nnc(-c2ccccc2)c(-c2ccccc2)n1)C(=O)N(C)C] | 2-[(5,6-diphenyl-1,2,4-triazin-3- yl)sulfanyl]-N,N-dimethyl- butanamide |
| B-07 | 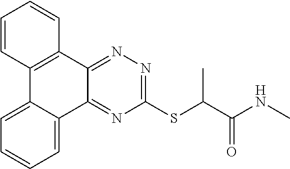 [SMILES: CNC(=O)C(C)Sc1nnc2c3ccccc3c3ccccc3c2n1] | N-methyl-2-phenanthro[9,10- e][1,2,4]triazine-3-ylsulfanyl- propanamide |
| B-11 | 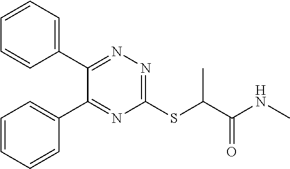 [SMILES: CNC(=O)C(C)Sc1nnc(-c2ccccc2)c(-c2ccccc2)n1] | 2-[(5,6-diphenyl-1,2,4-triazin-3- yl)sulfanyl]-N-methyl-propanamide |
| B-14 | 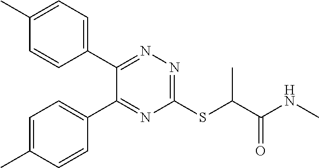 | 2-[5,6-bis(p-tolyl)-1,2,4-triazin-3- yl]sulfanyl]-N-methyl-propanamide |
| B-15 | 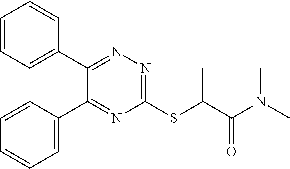 [SMILES: CC(Sc1nnc(-c2ccccc2)c(-c2ccccc2)n1)C(=O)N(C)C] | 2-[(5,6-diphenyl-1,2,4-triazin-3- yl)sulfanyl]-N,N-dimethyl- propanamide |
| B-18 | 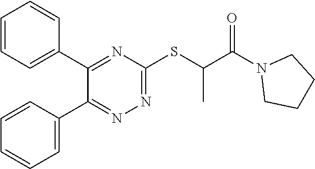 [SMILES: CC(Sc1nnc(-c2ccccc2)c(-c2ccccc2)n1)C(=O)N1CCCC1] | 2-[(5,6-diphenyl-1,2,4-triazin-3- yl)sulfanyl]-1-pyrrolidin-1-yl- propan-1-one |
| B-21 | 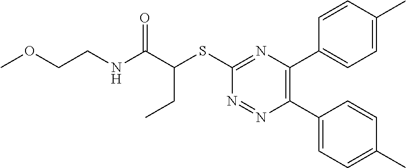 | 2-[[5,6-bis(p-tolyl)-1,2,4-triazin-3- yl]sulfanyl]-N-(2- methoxyethyl)butanamide |
| B-22 | 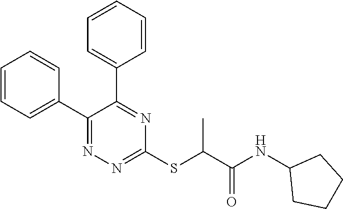 [SMILES: CC(Sc1nnc(-c2ccccc2)c(-c2ccccc2)n1)C(=O)NC1CCCC1] | N-cyclopentyl-2-[(5,6-diphenyl- 1,2,4-triazin-3- yl)sulfanyl]propanamide |
| B-28 | 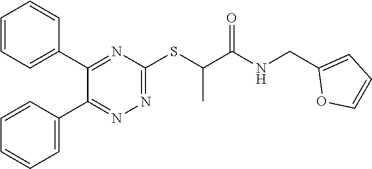 [SMILES: CC(Sc1nnc(-c2ccccc2)c(-c2ccccc2)n1)C(=O)NCc1ccco1] | 2-[(5,6-diphenyl-1,2,4-triazin-3- yl)sulfanyl]-N-(2- furylmethyl)propanamide |
| B-29 | 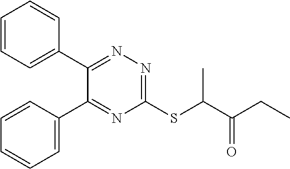 [SMILES: CCC(=O)C(C)Sc1nnc(-c2ccccc2)c(-c2ccccc2)n1] | 2-[(5,6-diphenyl-1,2,4-triazin-3- yl)sulfanyl]pentan-3-one |
| B-32 | 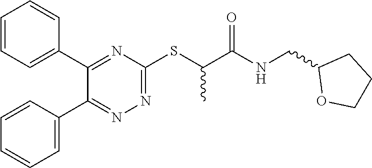 [SMILES: CC(Sc1nnc(-c2ccccc2)c(-c2ccccc2)n1)C(=O)NCC1CCCO1] | 2-[(5,6-diphenyl-1,2,4-triazin-3- yl)sulfanyl]-N-(tetrahydrofuran-2- ylmethyl)propanamide |
| B-34 | 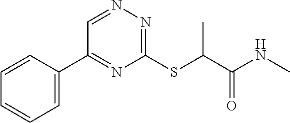 [SMILES: CNC(=O)C(C)Sc1nncc(-c2ccccc2)n1] | N-methyl-2-[(5-phenyl-1,2,4- triazin-3-yl)sulfanyl]propanamide |
| B-38 | 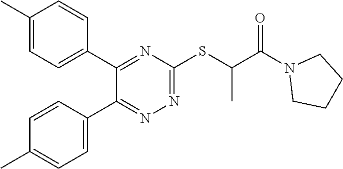 [SMILES: Cc1ccc(-c2nnc(SC(C)C(=O)N3CCCC3)nc2-c2ccc(C)cc2)cc1] | 2-[[5,6-bis(p-tolyl)-1,2,4-triazin-3- yl]sulfanyl]-1-pyrrolidin-1-yl- propan-1-one |
| B-39 | 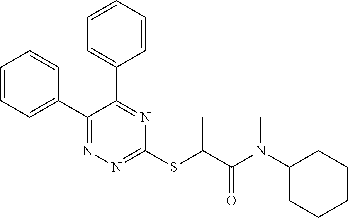 [SMILES: CC(Sc1nnc(-c2ccccc2)c(-c2ccccc2)n1)C(=O)N(C)C1CCCCC1] | N-cyclohexyl-2-[(5,6-diphenyl- 1,2,4-triazin-3-yl)sulfanyl]-N- methyl-propanamide |
| B-40 | 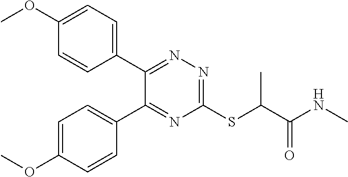 | 2-[[5,6-bis(4-methoxyphenyl)- 1,2,4-triazin-3-yl]sulfanyl]-N- methyl-propanamide |
| B-44 | 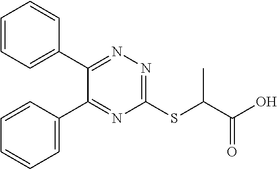 [SMILES: CC(Sc1nnc(-c2ccccc2)c(-c2ccccc2)n1)C(=O)O] | 2-[(5,6-diphenyl-1,2,4-triazin-3- yl)sulfanyl]propanoic acid |
| B-45 | 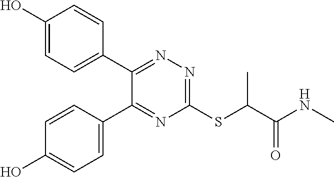 [SMILES: CNC(=O)C(C)Sc1nnc(-c2ccc(O)cc2)c(-c2ccc(O)cc2)n1] | 2-[[5,6-bis(4-hydroxyphenyl)- 1,2,4-triazin-3-yl]sulfanyl]-N- methyl-propanamide |
| B-48 | 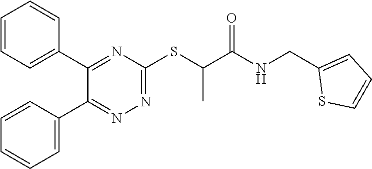 [SMILES: CC(Sc1nnc(-c2ccccc2)c(-c2ccccc2)n1)C(=O)NCc1cccs1] | 2-[(5,6-diphenyl-1,2,4-triazin-3- yl)sulfanyl]-N-(2- thienylmethyl)propanamide |
| B-59 | 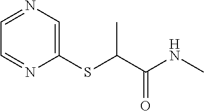 | N-methyl-2-pyrazine-2-ylsulfanyl- propanamide |
| B-60 | 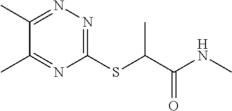 | 2-[(5,6-dimethyl-1,2,4-triazin-3- yl)sulfanyl]-N-methyl-propanamide |
| B-61 | 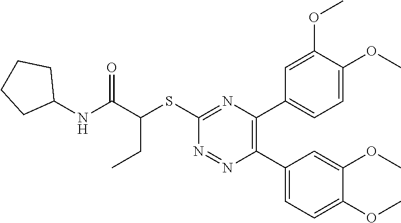 [SMILES: CCC(Sc1nnc(-c2ccc(OC)c(OC)c2)c(-c2ccc(OC)c(OC)c2)n1)C(=O)NC1CCCC1] | 2-[[5,6-bis(3,4-dimethoxyphenyl)- 1,2,4-triazin-3-yl]sulfanyl]-N- cyclopentyl-butanamide |
| B-65 | 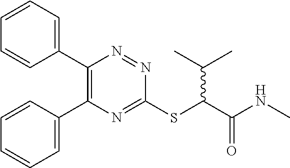 [SMILES: CNC(=O)C(Sc1nnc(-c2ccccc2)c(-c2ccccc2)n1)C(C)C] | 2-[(5,6-diphenyl-1,2,4-triazin-3- yl)sulfanyl]-N,3-dimethyl- butanamide |
| B-66 | 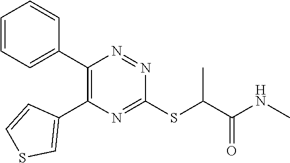 [SMILES: CNC(=O)C(C)Sc1nnc(-c2ccccc2)c(-c2ccsc2)n1] | N-methyl-2-[[6-phenyl-5-(3- thienyl)-1,2,4-triazin-3- yl]sulfanyl]propanamide |
| B-67 | 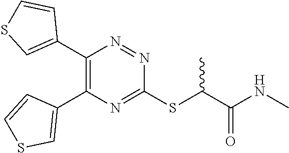 [SMILES: CNC(=O)C(C)Sc1nnc(-c2ccsc2)c(-c2ccsc2)n1] | 2-[[5,6-bis(3-thienyl)-1,2,4-triazin- 3-yl]sulfanyl]-N-methyl- propanamide |
| B-68 | 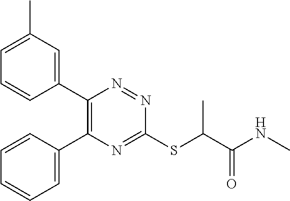 [SMILES: CNC(=O)C(C)Sc1nnc(-c2cccc(C)c2)c(-c2ccccc2)n1] | N-methyl-2-[6-(m-tolyl)-5-phenyl- 1,2,4-triazin-3- yl]sulfanyl]propanamide |
| B-69 | 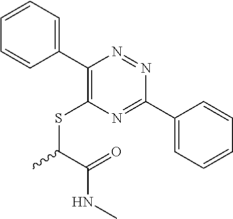 [SMILES: CNC(=O)C(C)Sc1nc(-c2ccccc2)nnc1-c1ccccc1] | 2-[(3,6-diphenyl-1,2,4-triazin-5- yl)sulfanyl]-N-methyl-propanamide |
| B-70 | 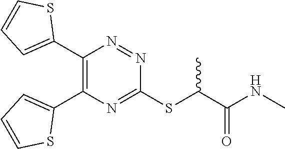 [SMILES: CNC(=O)C(C)Sc1nnc(-c2cccs2)c(-c2cccs2)n1] | 2-[[5,6-bis(2-thienyl)-1,2,4-triazin- 3-yl]sulfanyl]-N-methyl- propanamide |
| B-71 | 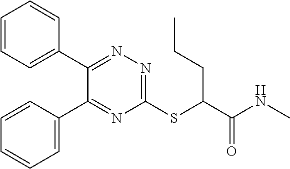 [SMILES: CCCC(Sc1nnc(-c2ccccc2)c(-c2ccccc2)n1)C(=O)NC] | 2-[(5,6-diphenyl-1,2,4-triazin-3- yl)sulfanyl]-N-methyl-pentanamide |
| B-72 | 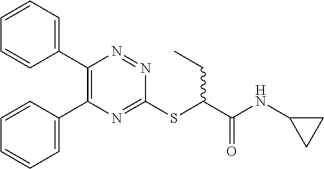 [SMILES: CCC(Sc1nnc(-c2ccccc2)c(-c2ccccc2)n1)C(=O)NC1CC1] | N-cyclopropyl-2-[(5,6-diphenyl- 1,2,4-triazin-3- yl)sulfanyl]butanamide |
| B-73 | 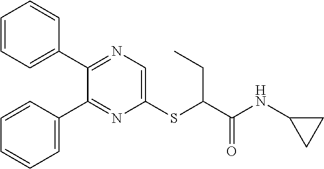 [SMILES: CCC(Sc1cnc(-c2ccccc2)c(-c2ccccc2)n1)C(=O)NC1CC1] | N-cyclopropyl-2-(5,6- diphenylpyrazin-2-yl)sulfanyl- butanamide |
| B-74 | 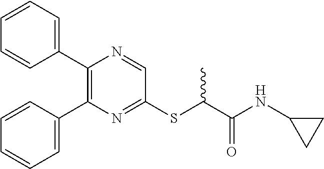 [SMILES: CC(Sc1cnc(-c2ccccc2)c(-c2ccccc2)n1)C(=O)NC1CC1] | N-cyclopropyl-2-(5,6- diphenylpyrazin-2-yl)sulfanyl- propanamide |
| B-75 | 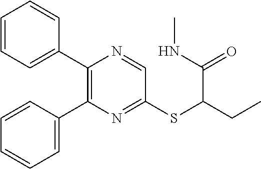 [SMILES: CCC(Sc1cnc(-c2ccccc2)c(-c2ccccc2)n1)C(=O)NC] | 2-[(5,6-Diphenylpyrazin-2- yl)sulfanyl]-N-methylbutanamide |
| B-76 | 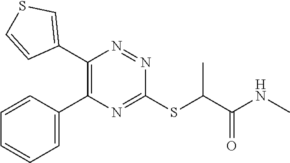 [SMILES: CNC(=O)C(C)Sc1nnc(-c2ccsc2)c(-c2ccccc2)n1] | N-methyl-2-[5-phenyl-6-(3- thienyl)-1,2,4-triazin-3- yl]sulfanyl]propanamide |
| B-77 | 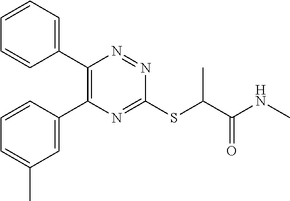 [SMILES: CNC(=O)C(C)Sc1nnc(-c2ccccc2)c(-c2cccc(C)c2)n1] | N-methyl-2-[[5-(m-tolyl)-6-phenyl- 1,2,4-triazin-3- yl]sulfanyl]propanamide |

and salts thereof, in particular acid addition salts, with inorganic or organic acids, wherein the coolants may be present in stereoisomerically pure form or as mixtures of different stereoisomers, preferably wherein the physiological coolant is selected from the group consisting of the compounds B-01, B-02, B-03, B-05, B-07, B-11 and B-18;
or
wherein the physiological coolant of the general formulae (VIIa) or (VIIIa) is selected from the group consisting of the compounds according to the following Table B:
TABLE B

| Compound | Structure | IUPAC name |
|---|---|---|
| A-01 | 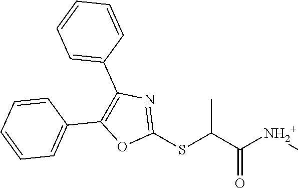 [SMILES: C[NH2+]C(=O)C(C)Sc1nc(-c2ccccc2)c(-c2ccccc2)o1] | 3-carboxy-3,5-dihydroxy-5- oxo-pentanoate;2-(4,5- diphenyloxazol-2- yl)sulfanylpropanoyl-methyl- ammonium |
| | 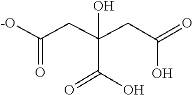 | |
| A-02 | 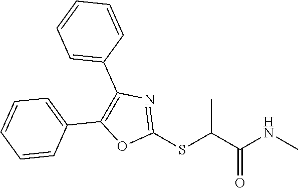 [SMILES: CNC(=O)C(C)Sc1nc(-c2ccccc2)c(-c2ccccc2)o1] | 2-(4,5-diphenyloxazol-2- yl)sulfanyl-N-methyl- propanamide |
| A-03 | 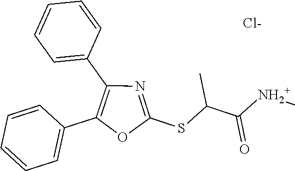 [SMILES: C[NH2+]C(=O)C(C)Sc1nc(-c2ccccc2)c(-c2ccccc2)o1 ; [Cl-]] | 2-(4,5-diphenyloxazol-2- y1)sulfanylpropanoyl-methyl- ammonium;chloride |
| A-05 | 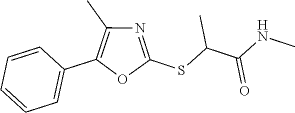 [SMILES: CNC(=O)C(C)Sc1nc(C)c(-c2ccccc2)o1] | N-methyl-2-(4-methyl-5- phenyl-oxazol-2-yl)sulfanyl- propanamide |
| A-09 | 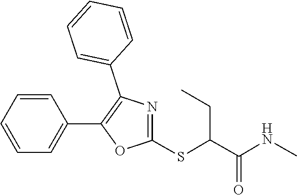 [SMILES: CCC(Sc1nc(-c2ccccc2)c(-c2ccccc2)o1)C(=O)NC] | 2-(4,5-diphenyloxazol-2- yl)sulfanyl-N-methyl- butanamide |
| A-10 | 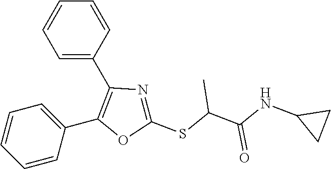 [SMILES: CC(Sc1nc(-c2ccccc2)c(-c2ccccc2)o1)C(=O)NC1CC1] | N-cyclopropyl-2-(4,5- diphenyloxazol-2-yl)sulfanyl- propanamide |
| A-12 | 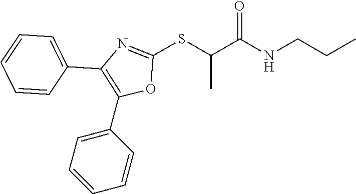 [SMILES: CCCNC(=O)C(C)Sc1nc(-c2ccccc2)c(-c2ccccc2)o1] | 2-(4,5-diphenyloxazol-2- yl)sulfanyl-N-propyl- propanamide |
| A-20 | 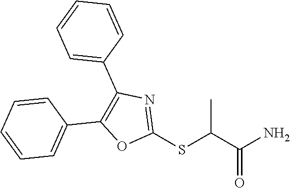 [SMILES: CC(Sc1nc(-c2ccccc2)c(-c2ccccc2)o1)C(N)=O] | 2-(4,5-diphenyloxazol-2- yl)sulfanylpropanamide |
| A-25 | 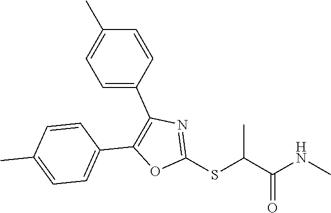 | 2-[4,5-bis(p-tolyl)oxazol-2- yl]sulfanyl-N-methyl- propanamide |
| A-28 | 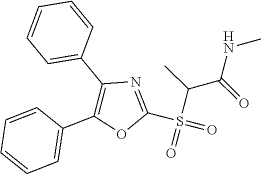 [SMILES: CNC(=O)C(C)S(=O)(=O)c1nc(-c2ccccc2)c(-c2ccccc2)o1] | 2-(4,5-diphenyloxazol-2- yl)sulfonyl-N-methyl- propanamide |
| A-30 | 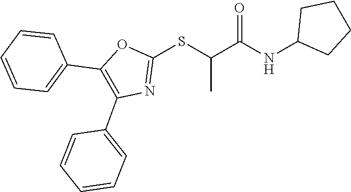 [SMILES: CC(Sc1nc(-c2ccccc2)c(-c2ccccc2)o1)C(=O)NC1CCCC1] | N-cyclopentyl-2-(4,5- diphenyloxazol-2-yl)sulfanyl- propanamide |
| A-33 | 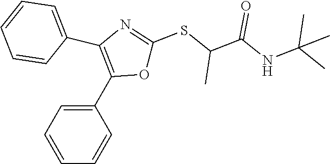 [SMILES: CC(Sc1nc(-c2ccccc2)c(-c2ccccc2)o1)C(=O)NC(C)(C)C] | N-tert-butyl-2-(4,5- diphenyloxazol-2-yl)sulfanyl- propanamide |
| A-37 | 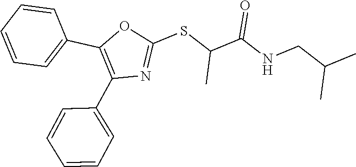 [SMILES: CC(C)CNC(=O)C(C)Sc1nc(-c2ccccc2)c(-c2ccccc2)o1] | 2-(4,5-diphenyloxazol-2- yl)sulfanyl-N-isobutyl- propanamide |
| A-47 | 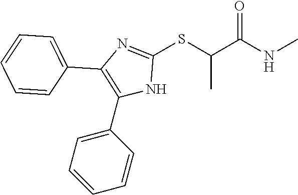 [SMILES: CNC(=O)C(C)Sc1nc(-c2ccccc2)c(-c2ccccc2)[nH]1] | 2-[(4,5-diphenyl-1H- imidazol-2-yl)sulfanyl]-N- methyl-propanamide |
| A-48 | 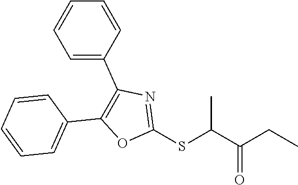 [SMILES: CCC(=O)C(C)Sc1nc(-c2ccccc2)c(-c2ccccc2)o1] | 2-(4,5-diphenyloxazol-2- yl)sulfanylpentan-3-one |
| A-50 | 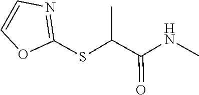 | N-methyl-2-oxazol-2- ylsulfanyl-propanamide |
| A-56 | 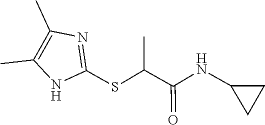 [SMILES: Cc1nc(SC(C)C(=O)NC2CC2)[nH]c1C] | N-cyclopropyl-2-[(4,5- dimethyl-1H-imidazol-2- yl)sulfanyl]propanamide |
| A-69 | 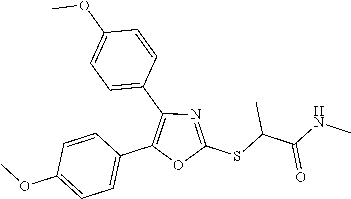 | 2-[4,5-bis(4- methoxyphenyl)oxazol-2- yl]sulfanyl-N-methyl- propanamide |

and salts thereof, in particular acid addition salts, with inorganic or organic acids, wherein the coolants may be present in stereoisomerically pure form or as mixtures of different stereoisomers, preferably wherein the physiological coolant is selected from the group consisting of the compounds A-02, A-05, A-09, A-10, and A-12.

4. Physiological coolant of the general formula (I) 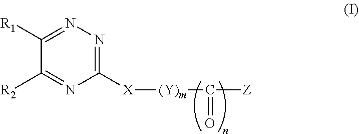
or the general formula (II) 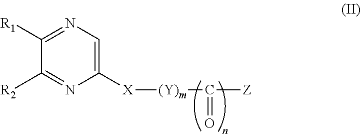
or the general formula (III) 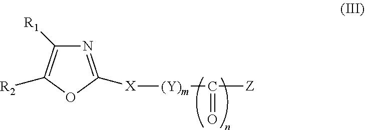
or the general formula (IV) 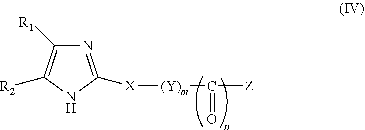
where in each of the formulae (I) to (IV)
the radicals R1 and R2 may be identical or different and have the following meanings independently of one another:
R1 H; or
a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group —R(C═O)—; or
an optionally substituted cycloalkyl group; or
an optionally substituted aryl group; or
an optionally substituted heterocycloalkyl group; or
is an optionally substituted heteroaryl group;
R2 H; or
a group Q; or
an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is an optionally substituted heteroaryl group;
or R1 and R2 together with the C atoms to which they are bonded form a conjugated or non-conjugated ring system;
X S; or
SO; or
SO₂; or
NH; or
O; or
an optionally substituted linear or branched alkyl group
an optionally substituted linear or branched alkenyl group
an optionally substituted linear or branched alkylthio group; or
an optionally substituted cycloalkyl group; or
an optionally substituted heterocycloalkyl group; or
an optionally substituted aryl group; or
is an optionally substituted heteroaryl group;
Y is an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—; or
an optionally substituted alkylaryl group; or
is an optionally substituted alkylheteroaryl group;
Z NH₂; or
an NHRa group; or
an NraRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
m is 0 or 1; and/or
n is 0 or 1;
with the proviso that
if m=0, Y is omitted; or
if m=1, Y occurs once in each case; or
if n=0, —(C═O)— is omitted; or
if n=1, —(C═O)— occurs once; or
if m=0 and n=0, Y is omitted and —(C═O)— is omitted; or
if m=1 and n=1, Y occurs once and —(C═O)— occurs once; or
if m=0 and n=1, Y is omitted and —(C═O) occurs once; or
if m=1 and n=0, Y occurs once and —(C═O)— is omitted;
wherein
the group Q is a radical selected from the group consisting of: halogen, —OA, —SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
wherein
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁-C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group,
is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
is in particular an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
Ra and/or Rb an optionally substituted linear or branched alkyl group; preferably a C1 to C3 alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
wherein the substituents are selected independently of one another;
and their salts, in particular acid addition salts, with inorganic or organic acids, whereby the coolants may be present in stereoisomerically pure form or as mixtures of different stereoisomers.

5. Physiological coolant according to claim 4, wherein in the general formulae (I) and (II)
X stands for S; and/or
Y represents an optionally substituted linear or branched alkyl group; and/or
m stands for 0 or 1; and/or
or
wherein in the general formulae (III) and (IV)
X is S or SO₂ or an optionally substituted linear or branched alkyl group or an optionally substituted cycloalkyl group or piperidinyl; and/or
Y represents an optionally substituted linear or branched alkyl group; and/or
m stands for 0 or 1.

6. Physiological coolant according to claim 4, represented by the general formula (V) 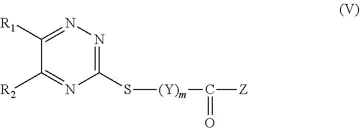
or by the general formula (VI) 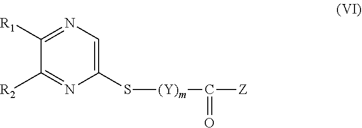
where in formulae (V) and (VI) in each case
the radicals R1 and R2 may be identical or different and have the following meanings independently of one another:
R1 as defined in claim 4;
R2 as defined in claim 4;
Y is an optionally substituted linear or branched alkyl group; or
an optionally substituted linear or branched alkenyl group; or
an optionally substituted linear or branched alkynyl group; or
an optionally substituted linear or branched alkoxy group; or
an optionally substituted acyl group R—(C═O)—; or
an optionally substituted alkylaryl group; or
is an optionally substituted alkylheteroaryl group;
Z NH₂; or
an NHRa group; or
an NraRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
m is 0 or 1;
with the proviso that
if m=0, Y is omitted; or
if m=1, Y occurs once in each case;
wherein
the group Q is a radical selected from the group consisting of: halogen, —OA, —SA, —NBB, —CF₃, —CN, —OCN, —SCN, —NO, —NO₂, —S(O)₂^{a}, —S(O)₂OA, —OS(O)₂^{a}, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁-C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group,
is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
Ra and/or Rb an optionally substituted linear or branched alkyl group, preferably a C₁ to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together and form a saturated or unsaturated ring;
wherein the substituents are selected independently of one another;
and their salts, in particular acid addition salts, with inorganic or organic acids, wherein the coolants may be present in stereoisomerically pure form or as mixtures of different stereoisomers;
or
given by the general formula (VII) 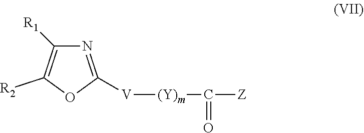
or by the general formula (VIII) 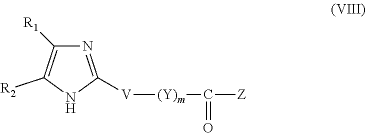
where in each of the formulae (VII) and (VIII)
the radicals R1 and R2 may be identical or different and have the following meanings independently of one another:
R1 as defined in claim 4;
R2 as defined in claim 4;
V S; or
SO₂; or
an optionally substituted linear or branched alkyl group; or
an optionally substituted cycloalkyl group; or
is piperidinyl;
Y is an optionally substituted linear or branched alkyl group; or
an optionally substituted linear or branched alkenyl group; or
an optionally substituted linear or branched alkynyl group; or
an optionally substituted linear or branched alkoxy group; or
is an optionally substituted acyl group R—(C═O)—;
Z NH₂; or
an NHRa group; or
an NraRb group; or
an optionally substituted linear or branched alkyl group; or
an optionally substituted linear or branched alkenyl group; or
an optionally substituted linear or branched alkylthio group; or
an optionally substituted linear or branched alkoxy group; or
OH; or
an optionally substituted cycloalkyl group; or
an optionally substituted heterocycloalkyl group; or
an optionally substituted aryl group; or
is an optionally substituted heteroaryl group; or
NH and together with the X forms a heterocyclic ring;
m is 0 or 1;
with the proviso that
if m=0, Y is omitted; or
if m=1, Y occurs once in each case;
wherein
the group Q is a radical selected from the group consisting of: halogen, —OA, —SA, —NBB, —CF₃, —CN, —OCN, —SCN, —NO, —NO₂, —S(O)₂A, —S(O)₂OA, —OS(O)₂A, —OS(O)₂OA, —P(O)(OA)₂, —P(O)(OA)(OA), —C(O)A, —C(S)A, —C(NA)A, —C(O)OA, —C(S)OA, —C(O)NBB, —C(NA)NBB, —OC(O)A, —OC(S)A, —OC(O)OA, —OC(S)OA, —NAC(O)A, —NAC(S)A, —NAC(O)OA, —NAC(S)OA, —NAC(O)NBB, —NAC(NA)A or —NAC(NA)NBB;
whereby
A is selected from the group consisting of:
Hydrogen,
optionally substituted linear or branched alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted linear or branched alkoxy group, in particular optionally substituted C₁- to C₆-alkoxy group, in particular optionally substituted C₁-, C₂-, C₃- or C₄-alkoxy group,
optionally substituted linear or branched alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
B represents A or alternatively, two B's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur;
the optionally substituted linear or branched alkyl group is an optionally substituted C₁- to C₁₀-alkyl group, in particular an optionally substituted C₁- to C₆-alkyl group, is in particular an optionally substituted C₁-, C₂-, C₃- or C₄-alkyl group;
the optionally substituted linear or branched alkenyl group is an optionally substituted C₁- to C₁₀-alkenyl group, in particular an optionally substituted C₁- to C₆-alkenyl group, is an optionally substituted C₁-, C₂-, C₃- or C₄-alkenyl group;
the optionally substituted linear or branched alkynyl group is an optionally substituted C₁- to C₁₀-alkynyl group, in particular an optionally substituted C₁- to C₆-alkynyl group, is in particular an optionally substituted C₁-, C₂-, C₃- or C₄-alkynyl group;
the optionally substituted linear or branched alkoxy group is an optionally substituted C₁- to C₁₀-alkoxy group, in particular an optionally substituted C₁- to C₆-alkoxy group, is in particular an optionally substituted C₁-, C₂-, C₃- or C₄-alkoxy group;
the optionally substituted linear or branched alkylthio group is an optionally substituted C₁- to C₁₀-alkylthio group, in particular an optionally substituted C₁- to C₆-alkylthio group,
is in particular an optionally substituted C₁-, C₂-, C₃- or C₄-alkylthio group;
in the acyl group R—(C═O)— the radical R represents hydrogen or an optionally substituted linear or branched C₁- to C₁₀-alkyl group, in particular an optionally substituted C₁- to C₆-alkyl group,
in particular represents an optionally substituted C₁-, C₂-, C₃- or C₄-acyl group;
the optionally substituted cycloalkyl group is an optionally substituted three- to ten-membered, in particular three-, four-, five-, six- or seven-membered, monocyclic cycloalkyl group or an optionally substituted nine- to twelve-membered polycyclic, in particular bicyclic, cycloalkyl group;
the optionally substituted aryl group is an optionally substituted three- to ten-membered, in particular three-, four-, five-, six- or seven-membered, monocyclic aryl group or an optionally substituted nine- to twelve-membered polycyclic, in particular bicyclic aryl group;
the optionally substituted heterocycloalkyl group is an optionally substituted three- to ten-membered, in particular three-, four-, five-, six- or seven-membered monocyclic heterocycloalkyl group or an optionally substituted nine- to twelve-membered polycyclic, in particular bicyclic, heterocycloalkyl group, wherein the heterocycloalkyl group may comprise one, two, three or four identical or different heteroatoms selected from the group consisting of nitrogen, oxygen and sulfur;
the optionally substituted heteroaryl group is an optionally substituted three- to ten-membered, in particular three-, four-, five-, six- or seven-membered monocyclic heteroaryl group or an optionally substituted nine- to twelve-membered polycyclic, in particular bicyclic, heteroaryl group, wherein the heteroaryl group may comprise one, two, three or four identical or different heteroatoms selected from the group consisting of nitrogen, oxygen and sulfur;
Ra and/or Rb an optionally substituted linear or branched alkyl group; preferably a C₁ to C₃ alkyl group, preferably a methyl group; or
an optionally substituted linear or branched alkenyl group; or
an optionally substituted linear or branched alkynyl group; or
an optionally substituted linear or branched alkoxy group; or
an optionally substituted linear or branched alkylthio group; or
an optionally substituted acyl group R—(C═O)—; or
an optionally substituted cycloalkyl group; or
an optionally substituted aryl group; or
an optionally substituted heterocycloalkyl group; or
is/are an optionally substituted heteroaryl group; or
Ra and Rb, as defined above, are linked together, and form a saturated or unsaturated ring;
wherein the substituents are selected independently of one another;
and their salts, in particular acid addition salts, with inorganic or organic acids, wherein the coolants may be present in stereoisomerically pure form or as mixtures of different stereoisomers.

7. Physiological coolant according to claim 1, wherein furthermore the optionally substituted groups mentioned above have one or more substituents, such that:
(i) when in the aforementioned substituted groups the substitution occurs on a saturated carbon, the substituent on the saturated carbon is selected from the group consisting of: —X, halogen, ═O, —OY, —SiR₃, —SY, ═S, —NZZ, ═NY, ═N—OY, —CF₃, —CN, —OCN, —SCN, —NO, —NO₂, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ;
(ii) when in the aforementioned substituted groups the substitution occurs on an unsaturated carbon, the substituent on the unsaturated carbon is selected from the group consisting of: —X, halogen, —OY, —SiR₃, —SY, —NZZ, —CF₃, —CN, —OCN, —SCN, —NO, —NO₂, —S(O)₂Y, —S(O)₂OY, —OS(O)₂Y, —OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ;
(iii) when in the above substituted groups the substitution occurs on a nitrogen atom, the substituent on the nitrogen atom is selected from the group consisting of: —X, —OY, —SY, —NZZ, —CF₃, —CN, —OCN, —SCN, —NO, —NO₂, —S(O)₂Y, —S(O)₂OY, —OS(O)₂Y, -OS(O)₂OY, —P(O)(OY)₂, —P(O)(OY)(OY), —C(O)Y, —C(S)Y, —C(NY)Y, —C(O)OY, —C(S)OY, —C(O)NZZ, —C(NY)NZZ, —OC(O)Y, —OC(S)Y, —OC(O)OY, —OC(S)OY, —NYC(O)Y, —NYC(S)Y, —NYC(O)OY, —NYC(S)OY, —NYC(O)NZZ, —NYC(NY)Y and —NYC(NY)NZZ;
where in cases (i), (ii) and (iii)
X is selected from the group consisting of: optionally substituted alkyl group, in particular optionally substituted C₁- to C₁₀-alkyl group, in particular optionally substituted C₁- to C₆-alkyl group, in particular optionally substituted C₁-, C₂-, C₃- or C₄-alkyl group,
optionally substituted alkoxy group, in particular optionally substituted C₁-C₆-alkoxy group, in particular optionally substituted C₁-, C₂-, C₃- or C₄-alkoxy group, optionally substituted alkylthio group, in particular optionally substituted C₁- to C₆-alkylthio group, in particular optionally substituted C₁-, C₂-, C₃- or C₄-alkylthio group,
optionally substituted cycloalkyl group,
optionally substituted aryl group,
optionally substituted heterocycloalkyl group,
optionally substituted heteroaryl group; and/or
Y is selected from the group consisting of: hydrogen or X; and/or
Z represents Y or alternatively two Z's together with the nitrogen atom to which they are attached represent a four, five, six or seven membered heterocycloalkyl ring or heteroaryl ring, wherein the heterocycloalkyl ring or heteroaryl ring may comprise one, two, three or four identical or different heteroatoms selected from the group consisting of nitrogen, oxygen and sulfur.

8. Physiological coolant according to claim 1, wherein in the general formulae (I) to (VIII) and (Va) to (VIIIa) the radicals R1 and R2 may be identical or different and independently of one another have the following meanings:
R1 is H or an optionally substituted C1- to C3-alkyl group or an optionally substituted phenyl group, preferably an optionally substituted phenyl group; and/or
R2 represents H or an optionally substituted C1 to C3 alkyl group or an optionally substituted phenyl group, preferably an optionally substituted phenyl group.

9. Physiological coolant according to claim 4, wherein in the general formulae (I) to (VIII) Y stands for a methyl group or a methyl group substituted with a methyl group, an ethyl group, a butyl group or a propyl group.

10. Physiological coolant according to claim 1, wherein in the general formulae (I) to (VIII) and (Va), (VIIa) and (VIIIa) Z is selected from the group consisting of: —NH₂, —NH—CH₃, —NH—CH₂—CH₃, —NH—CH₂—CH₂—CH₃, —NH—CH₂-CH₂—CH₂—CH₃, —NH—CH(CH₃)—CH(CH₃)₂, NH—CH(CH₃)—CH₂—CH₂—CH₃, —NH—CH₂—CH(CH₃)₂, —NH—CH₂—CH₂—O—CH₃, —NH—CH(CH₃)—CH₂—O—CH₃, —NH—C(═O)—CH₃, —NH— C(═O)—O—CH₃, —NH—CH(CH₃)—CH₂—OH, —NH—CH₂-furanyl, —NH—CH₂-tetrahydrofuranyl, —NH—CH₂-thiophenyl, —NH-toluolyl, —NH—CH—(CH₃)₂, —NH—C(CH₃)₃, —NH-cyclopropyl, —NH— cyclobutyl, —NH-cyclopentyl, —N(CH₃)₂, —N(CH₃)-cyclohexyl, —N(CH₂—CH₃)₂, azetidinyl, pyrrolidinyl, piperidinyl, azacyclobutadienyl, pyrrolyl, pyridinyl, —O, —OH, —O—CH₃, —O—C(═O)—CH₃, oxetanyl, —CH₃, —CH₂—CH₃, —CH(CH₃)₂, —C(OH)—CH₂—OH, cyclopropyl, phenyl, and —CH₂—S—CH₃; and wherein in the general formula (VIa) Z is selected from the group consisting of —NH₂, —NH—CH₃, —NH—CH₂—CH₃, —NH—CH₂—CH₂—CH₃, —NH—CH₂—CH₂—CH₂—CH₃, —NH—CH(CH₃)—CH(CH₃)₂, —NH—CH(CH₃)—CH₂—CH₂—CH₃, —NH—CH₂—CH(CH₃)₂, —NH— CH₂—CH₂—O—CH₃, —NH—CH(CH₃)—CH₂—O—CH₃, —NH—C(═O)—CH₃, —NH—C(═O)—O—CH₃, —NH— CH(CH₃)—CH₂—OH, —NH—CH₂-furanyl, —NH—CH₂-tetrahydrofuranyl, —NH—CH₂-thiophenyl, —NH-toluolyl, —NH—CH—(CH₃)₂, —NH—C(CH₃)₃, —NH-cyclopropyl, —NH-cyclobutyl, —NH— cyclopentyl, —N(CH₃)₂, —N(CH₃)-cyclohexyl, —N(CH₂—CH₃)₂, acetidinyl, pyrrolidinyl, piperidinyl, azacyclobutadienyl, pyrrolyl, pyridinyl, —O, —O—CH₃, —O—C(═O)—CH₃, oxetanyl, —CH₃, —CH₂—CH₃, —CH(CH₃)₂, —C(OH)—CH₂—OH, cyclopropyl, phenyl, and-CH₂—S—CH₃.

11. Physiological coolant according to claim 4, wherein the physiological coolant of general formulae (I), (II), (V) or (VI) is selected from the group consisting of the compounds according to Table 1 below:
TABLE 1

| Compound | Structure | IUPAC name |
|---|---|---|
| B-01 | 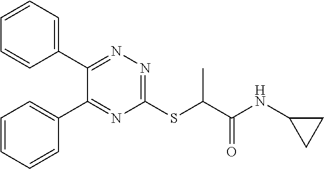 [SMILES: CC(Sc1nnc(-c2ccccc2)c(-c2ccccc2)n1)C(=O)NC1CC1] | N-Cyclopropyl-2-[(5,6-diphenyl- 1,2,4-triazin-3- yl)sulfanyl]propanamide |
| B-02 | 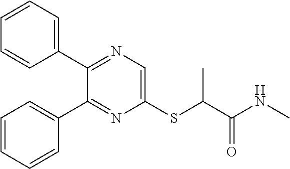 [SMILES: CNC(=O)C(C)Sc1cnc(-c2ccccc2)c(-c2ccccc2)n1] | 2-(5,6-diphenylpyrazin-2- yl)sulfanyl-N-methyl-propanamide |
| B-03 | 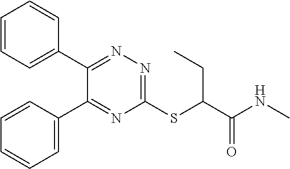 [SMILES: CCC(Sc1nnc(-c2ccccc2)c(-c2ccccc2)n1)C(=O)NC] | 2-[(5,6-diphenyl-1,2,4-triazin-3- yl)sulfany1]-N-methyl-butanamide |
| B-04 | 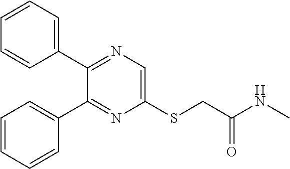 [SMILES: CNC(=O)CSc1cnc(-c2ccccc2)c(-c2ccccc2)n1] | 2-(5,6-diphenylpyrazin-2- yl)sulfanyl-N-methyl-acetamide |
| B-05 | 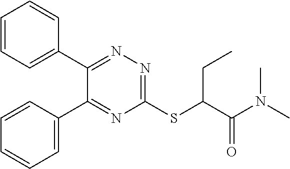 [SMILES: CCC(Sc1nnc(-c2ccccc2)c(-c2ccccc2)n1)C(=O)N(C)C] | 2-[(5,6-diphenyl-1,2,4-triazin-3- yl)sulfanyl]-N,N-dimethyl- butanamide |
| B-06 | 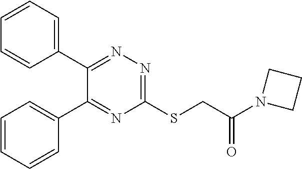 [SMILES: O=C(CSc1nnc(-c2ccccc2)c(-c2ccccc2)n1)N1CCC1] | 1-(azetidin-1-y1)-2-[(5,6-diphenyl- 1,2,4-triazin-3-yl)sulfanyllethanone |
| B-07 | 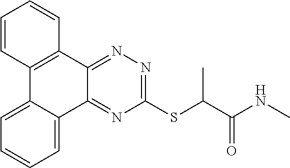 [SMILES: CNC(=O)C(C)Sc1nnc2c3ccccc3c3ccccc3c2n1] | N-methyl-2-phenanthro[9,10- e][1,2,4]triazine-3-ylsulfanyl- propanamide |
| B-11 | 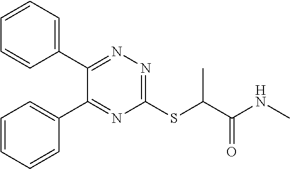 [SMILES: CNC(=O)C(C)Sc1nnc(-c2ccccc2)c(-c2ccccc2)n1] | 2-[(5,6-diphenyl-1,2,4-triazin-3- yl)sulfany1]-N-methyl-propanamide |
| B-14 | 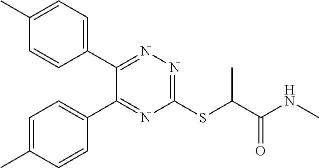 | 2-[[5,6-bis(p-tolyl)-1,2,4-triazin-3- yl]sulfanyl]-N-methyl-propanamide |
| B-15 | 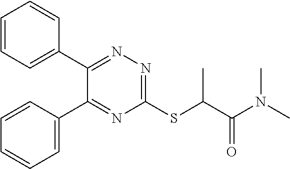 [SMILES: CC(Sc1nnc(-c2ccccc2)c(-c2ccccc2)n1)C(=O)N(C)C] | 2-[(5,6-diphenyl-1,2,4-triazin-3- yl)sulfanyl]-N,N-dimethyl- propanamide |
| B-17 | 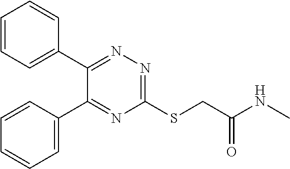 [SMILES: CNC(=O)CSc1nnc(-c2ccccc2)c(-c2ccccc2)n1] | 2-[(5,6-diphenyl-1,2,4-triazin-3- yl)sulfany1]-N-methyl-acetamide |
| B-18 | 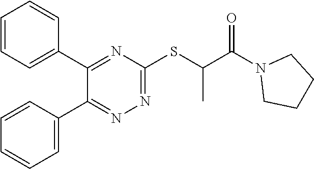 [SMILES: CC(Sc1nnc(-c2ccccc2)c(-c2ccccc2)n1)C(=O)N1CCCC1] | 2-[(5,6-diphenyl-1,2,4-triazin-3- yl)sulfany1]-1-pyrrolidin-1-yl-propan-1-one |
| B-19 | 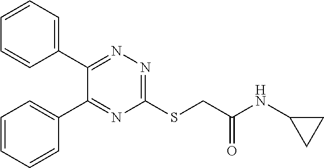 [SMILES: O=C(CSc1nnc(-c2ccccc2)c(-c2ccccc2)n1)NC1CC1] | N-cyclopropyl-2-[(5,6-diphenyl- 1,2,4-triazin-3- yl)sulfanyl]acetamide |
| B-21 | 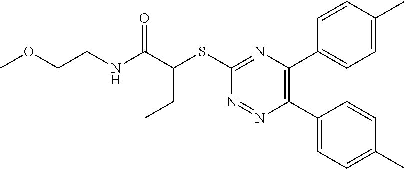 | 2-[[5,6-bis(p-tolyl)-1,2,4-triazin-3- yl]sulfany1]-N-(2- methoxyethyl)butanamide |
| B-22 | 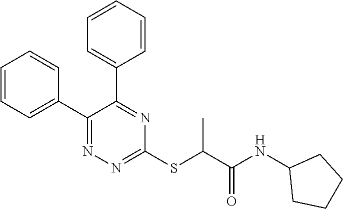 [SMILES: CC(Sc1nnc(-c2ccccc2)c(-c2ccccc2)n1)C(=O)NC1CCCC1] | N-cyclopentyl-2-[(5,6-diphenyl- 1,2,4-triazin-3- yl)sulfanyl]propanamide |
| B-23 | 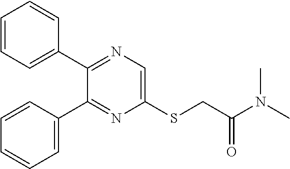 [SMILES: CN(C)C(=O)CSc1cnc(-c2ccccc2)c(-c2ccccc2)n1] | 2-(5,6-diphenylpyrazin-2- yl)sulfanyl-N,N-dimethyl- acetamide |
| B-24 | 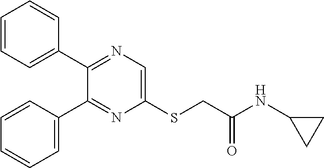 [SMILES: O=C(CSc1cnc(-c2ccccc2)c(-c2ccccc2)n1)NC1CC1] | N-cyclopropyl-2-(5,6- diphenylpyrazin-2-yl)sulfanyl- acetamide |
| B-25 | 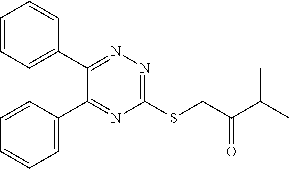 [SMILES: CC(C)C(=O)CSc1nnc(-c2ccccc2)c(-c2ccccc2)n1] | 1-[(5,6-diphenyl-1,2,4-triazin-3- yl)sulfanyl]-3-methyl-butan-2-one |
| B-26 | 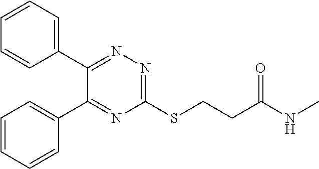 [SMILES: CNC(=O)CCSc1nnc(-c2ccccc2)c(-c2ccccc2)n1] | 3-[(5,6-diphenyl-1,2,4-triazin-3- yl)sulfanyl]-N-methyl-propanamide |
| B-27 | 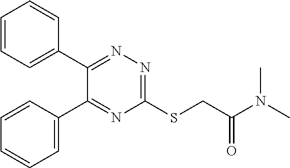 [SMILES: CN(C)C(=O)CSc1nnc(-c2ccccc2)c(-c2ccccc2)n1] | 2-[(5,6-diphenyl-1,2,4-triazin-3- yl)sulfany1]-N,N-dimethyl- acetamide |
| B-28 | 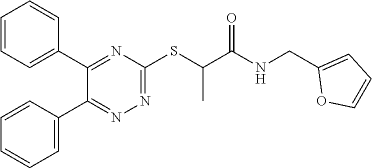 [SMILES: CC(Sc1nnc(-c2ccccc2)c(-c2ccccc2)n1)C(=O)NCc1ccco1] | 2-[(5,6-diphenyl-1,2,4-triazin-3- yl)sulfany1]-N-(2- furylmethyl)propanamide |
| B-29 | 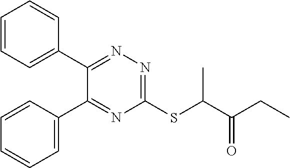 [SMILES: CCC(=O)C(C)Sc1nnc(-c2ccccc2)c(-c2ccccc2)n1] | 2-[(5,6-diphenyl-1,2,4-triazin-3- yl)sulfanyl]pentan-3-one |
| B-30 | 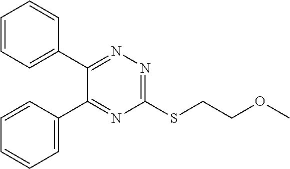 [SMILES: COCCSc1nnc(-c2ccccc2)c(-c2ccccc2)n1] | 3-(2-methoxyethylsulfanyl)-5,6- diphenyl-1,2,4-triazine |
| B-31 | 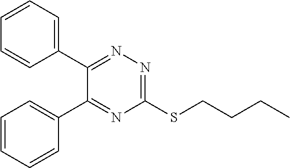 [SMILES: CCCCSc1nnc(-c2ccccc2)c(-c2ccccc2)n1] | 3-butylsulfanyl-5,6-diphenyl-1,2,4- triazine |
| B-32 | 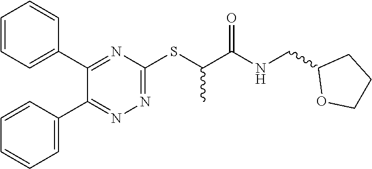 [SMILES: CC(Sc1nnc(-c2ccccc2)c(-c2ccccc2)n1)C(=O)NCC1CCCO1] | 2-[(5,6-diphenyl-1,2,4-triazin-3- yl)sulfany1]-N-(tetrahydrofuran-2- ylmethyl)propanamide |
| B-33 | 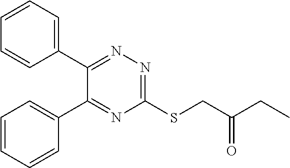 [SMILES: CCC(=O)CSc1nnc(-c2ccccc2)c(-c2ccccc2)n1] | 1-[(5,6-diphenyl-1,2,4-triazin-3- yl)sulfanyl]butan-2-one |
| B-34 | 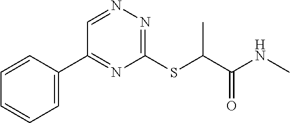 [SMILES: CNC(=O)C(C)Sc1nncc(-c2ccccc2)n1] | N-methyl-2-[(5-phenyl-1,2,4- triazin-3-yl)sulfanyl]propanamide |
| B-36 | 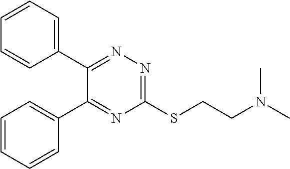 [SMILES: CN(C)CCSc1nnc(-c2ccccc2)c(-c2ccccc2)n1] | 2-[(5,6-diphenyl-1,2,4-triazin-3- yl)sulfany1]-N,N-dimethyl- ethanamine |
| B-37 | 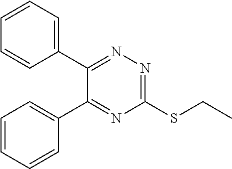 [SMILES: CCSc1nnc(-c2ccccc2)c(-c2ccccc2)n1] | 3-ethylsulfany1-5,6-diphenyl-1,2,4- triazine |
| B-38 | 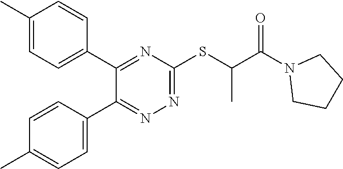 [SMILES: Cc1ccc(-c2nnc(SC(C)C(=O)N3CCCC3)nc2-c2ccc(C)cc2)cc1] | 2-[[5,6-bis(p-tolyl)-1,2,4-triazin-3- yl]sulfanyl]-1-pyrrolidin-1-yl- propan-1-one |
| B-39 | 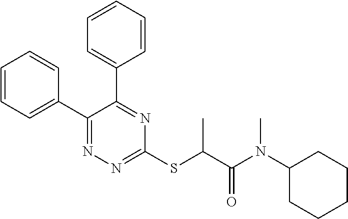 [SMILES: CC(Sc1nnc(-c2ccccc2)c(-c2ccccc2)n1)C(=O)N(C)C1CCCCC1] | N-cyclohexyl-2-[(5,6-diphenyl- 1,2,4-triazin-3-yl)sulfanyl]-N- methyl-propanamide |
| B-40 | 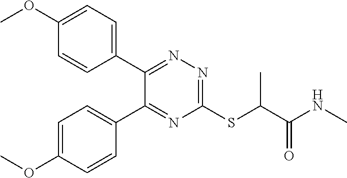 | 2-[[5,6-bis(4-methoxyphenyl)- 1,2,4-triazin-3-yl]sulfany1]-N- methyl-propanamide |
| B-41 | 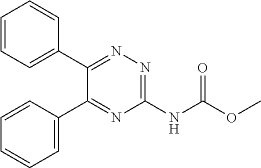 [SMILES: COC(=O)Nc1nnc(-c2ccccc2)c(-c2ccccc2)n1] | methyl N-(5,6-diphenyl-1,2,4- triazin-3-y1)carbamate |
| B-42 | 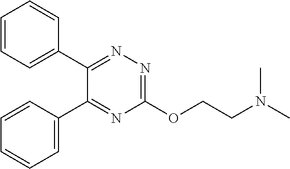 [SMILES: CN(C)CCOc1nnc(-c2ccccc2)c(-c2ccccc2)n1] | 2-[(5,6-diphenyl-1,2,4-triazin-3- yl)oxy]-N,N-dimethyl-ethanamine |
| B-43 | 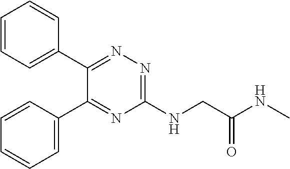 [SMILES: CNC(=O)CNc1nnc(-c2ccccc2)c(-c2ccccc2)n1] | 2-[(5,6-diphenyl-1,2,4-triazin-3- yl)amino]-N-methyl-acetamide |
| B-44 | 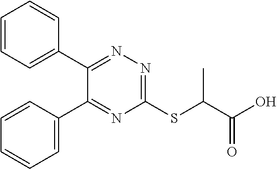 [SMILES: CC(Sc1nnc(-c2ccccc2)c(-c2ccccc2)n1)C(=O)O] | 2-[(5,6-diphenyl-1,2,4-triazin-3- yl)sulfanyl]propanoic acid |
| B-45 | 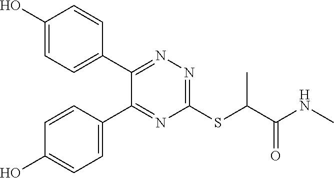 [SMILES: CNC(=O)C(C)Sc1nnc(-c2ccc(O)cc2)c(-c2ccc(O)cc2)n1] | 2-[5,6-bis(4-hydroxyphenyl)- 1,2,4-triazin-3-y1]sulfanyl]-N- methyl-propanamide |
| B-46 | 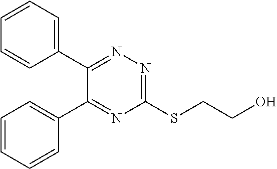 [SMILES: OCCSc1nnc(-c2ccccc2)c(-c2ccccc2)n1] | 2-[(5,6-diphenyl-1,2,4-triazin-3- yl)sulfanyl]ethanol |
| B-47 | 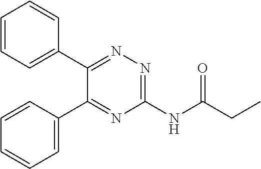 [SMILES: CCC(=O)Nc1nnc(-c2ccccc2)c(-c2ccccc2)n1] | N-(5,6-diphenyl-1,2,4-triazin-3- yl)propanamide |
| B-48 | 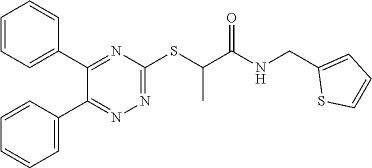 [SMILES: CC(Sc1nnc(-c2ccccc2)c(-c2ccccc2)n1)C(=O)NCc1cccs1] | 2-[(5,6-diphenyl-1,2,4-triazin-3- yl)sulfany1]-N-(2- thienylmethyl)propanamide |
| B-49 | 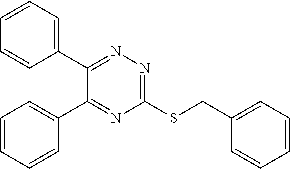 [SMILES: c1ccc(CSc2nnc(-c3ccccc3)c(-c3ccccc3)n2)cc1] | 3-benzylsulfany1-5,6-diphenyl- 1,2,4-triazine |
| B-50 | 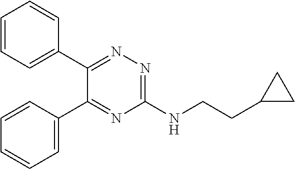 [SMILES: c1ccc(-c2nnc(NCCC3CC3)nc2-c2ccccc2)cc1] | N-(2-cyclopropylethyl)-5,6- diphenyl-1,2,4-triazin-3-amine |
| B-51 | 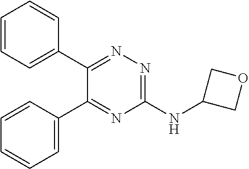 [SMILES: c1ccc(-c2nnc(NC3COC3)nc2-c2ccccc2)cc1] | N-(oxetan-3-y1)-5,6-diphenyl-1,2,4- triazin-3-amine |
| B-52 | 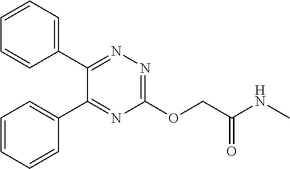 [SMILES: CNC(=O)COc1nnc(-c2ccccc2)c(-c2ccccc2)n1] | 2-[(5,6-diphenyl-1,2,4-triazin-3- yl)oxy]-N-methyl-acetamide |
| B-53 | 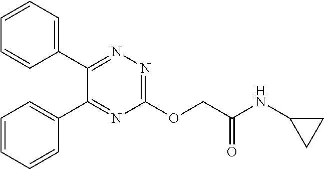 [SMILES: O=C(COc1nnc(-c2ccccc2)c(-c2ccccc2)n1)NC1CC1] | N-cyclopropyl-2-[(5,6-diphenyl- 1,2,4-triazin-3-yl)oxy ]acetamide |
| B-54 | 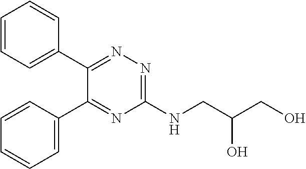 [SMILES: OCC(O)CNc1nnc(-c2ccccc2)c(-c2ccccc2)n1] | 3-[(5,6-diphenyl-1,2,4-triazin-3- yl)amino]propane-1,2-diol |
| B-55 | 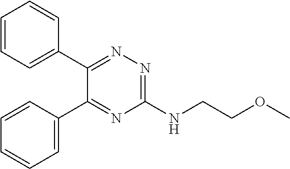 [SMILES: COCCNc1nnc(-c2ccccc2)c(-c2ccccc2)n1] | N-(2-methoxyethyl)-5,6-diphenyl- 1,2,4-triazin-3-amine |
| B-56 | 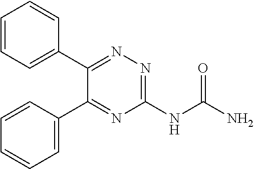 [SMILES: NC(=O)Nc1nnc(-c2ccccc2)c(-c2ccccc2)n1] | (5,6-diphenyl-1,2,4-triazin-3- yl)urea |
| B-57 | 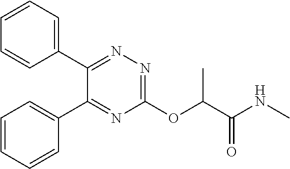 [SMILES: CNC(=O)C(C)Oc1nnc(-c2ccccc2)c(-c2ccccc2)n1] | 2-[(5,6-diphenyl-1,2,4-triazin-3- yl)oxy]-N-methyl-propanamide |
| B-58 | 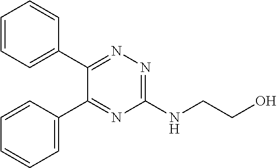 [SMILES: OCCNc1nnc(-c2ccccc2)c(-c2ccccc2)n1] | 2-[(5,6-diphenyl-1,2,4-triazin-3- yl)amino]ethanol |
| B-59 | 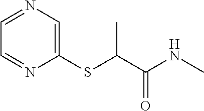 | N-methyl-2-pyrazine-2-ylsulfanyl- propanamide |
| B-60 | 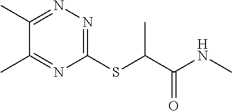 | 2-[(5,6-dimethyl-1,2,4-triazin-3- y1)sulfanyl]-N-methyl-propanamide |
| B-61 | 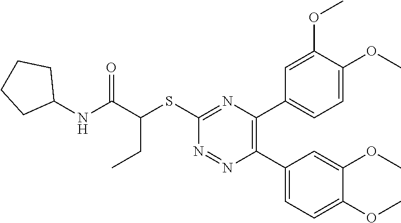 [SMILES: CCC(Sc1nnc(-c2ccc(OC)c(OC)c2)c(-c2ccc(OC)c(OC)c2)n1)C(=O)NC1CCCC1] | 2-[[5,6-bis(3,4-dimethoxyphenyl)- 1,2,4-triazin-3-yl]sulfany1]-N- cyclopentyl-butanamide |
| B-62 | 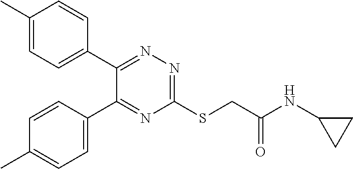 [SMILES: Cc1ccc(-c2nnc(SCC(=O)NC3CC3)nc2-c2ccc(C)cc2)cc1] | 2-[[5,6-bis(p-tolyl)-1,2,4-triazin-3- yl]sulfanyl]-N-cyclopropyl- acetamide |
| B-63 | 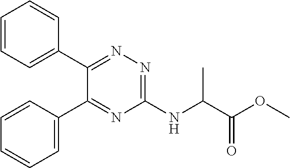 [SMILES: COC(=O)C(C)Nc1nnc(-c2ccccc2)c(-c2ccccc2)n1] | methyl 2-[(5,6-diphenyl-1,2,4- triazin-3-yl)amino]propanoate |
| B-64 | 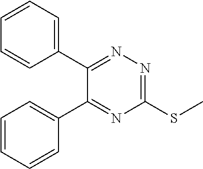 [SMILES: CSc1nnc(-c2ccccc2)c(-c2ccccc2)n1] | 3-methylsulfanyl-5,6-diphenyl- 1,2,4-triazine |
| B-65 | 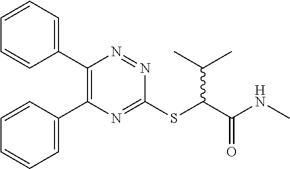 [SMILES: CNC(=O)C(Sc1nnc(-c2ccccc2)c(-c2ccccc2)n1)C(C)C] | 2-[(5,6-diphenyl-1,2,4-triazin-3- yl)sulfanyl]-N,3-dimethyl- butanamide |
| B-66 | 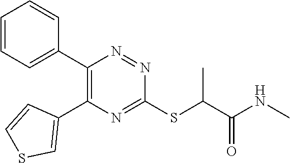 [SMILES: CNC(=O)C(C)Sc1nnc(-c2ccccc2)c(-c2ccsc2)n1] | N-methyl-2-[6-phenyl-5-(3- thienyl)-1,2,4-triazin-3- yl]sulfanyl]propanamide |
| B-67 | 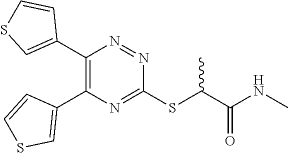 [SMILES: CNC(=O)C(C)Sc1nnc(-c2ccsc2)c(-c2ccsc2)n1] | 2-[[5,6-bis(3-thienyl)-1,2,4-triazin- 3-yl]sulfany1]-N-methyl- propanamide |
| B-68 | 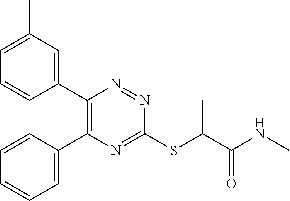 [SMILES: CNC(=O)C(C)Sc1nnc(-c2cccc(C)c2)c(-c2ccccc2)n1] | N-methyl-2-[[6-(m-toly1)-5-phenyl- 1,2,4-triazin-3- yl]sulfanyl]propanamide |
| B-69 | 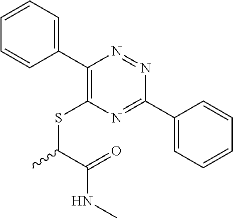 [SMILES: CNC(=O)C(C)Sc1nc(-c2ccccc2)nnc1-c1ccccc1] | 2-[(3,6-diphenyl-1,2,4-triazin-5- yl)sulfanyl]-N-methyl-propanamide |
| B-70 | 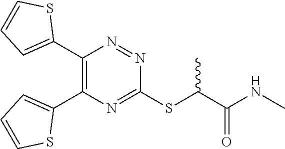 [SMILES: CNC(=O)C(C)Sc1nnc(-c2cccs2)c(-c2cccs2)n1] | 2-[[5,6-bis(2-thienyl)-1,2,4-triazin- 3-y1]sulfany1]-N-methyl- propanamide |
| B-71 | 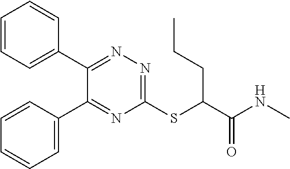 [SMILES: CCCC(Sc1nnc(-c2ccccc2)c(-c2ccccc2)n1)C(=O)NC] | 2-[(5,6-diphenyl-1,2,4-triazin-3- y1)sulfany1]-N-methyl-pentanamide |
| B-72 | 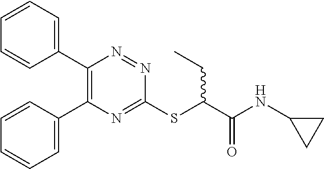 [SMILES: CCC(Sc1nnc(-c2ccccc2)c(-c2ccccc2)n1)C(=O)NC1CC1] | N-cyclopropyl-2-[(5,6-diphenyl- 1,2,4-triazin-3- y1)sulfanyl]butanamide |
| B-73 | 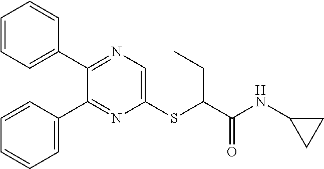 [SMILES: CCC(Sc1cnc(-c2ccccc2)c(-c2ccccc2)n1)C(=O)NC1CC1] | N-cyclopropyl-2-(5,6- diphenylpyrazin-2-yl)sulfanyl- butanamide |
| B-74 | 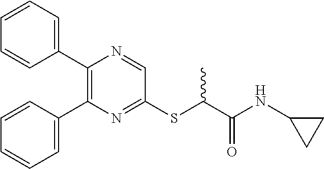 [SMILES: CC(Sc1cnc(-c2ccccc2)c(-c2ccccc2)n1)C(=O)NC1CC1] | N-cyclopropyl-2-(5,6- diphenylpyrazin-2-yl)sulfanyl- propanamide |
| B-75 | 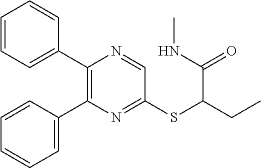 [SMILES: CCC(Sc1cnc(-c2ccccc2)c(-c2ccccc2)n1)C(=O)NC] | 2-[(5,6-Diphenylpyrazin-2- yl)sulfany1]-N-methylbutanamide |
| B-76 | 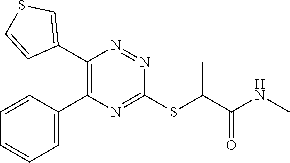 [SMILES: CNC(=O)C(C)Sc1nnc(-c2ccsc2)c(-c2ccccc2)n1] | N-methyl-2-[5-phenyl-6-(3- thieny1)-1,2,4-triazin-3- yl]sulfanyl]propanamide |
| B-77 | 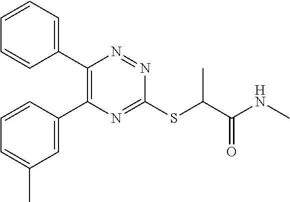 [SMILES: CNC(=O)C(C)Sc1nnc(-c2ccccc2)c(-c2cccc(C)c2)n1] | N-methyl-2-[5-(m-toly1)-6-phenyl- 1,2,4-triazin-3- yl]sulfanyl]propanamide |

and salts thereof, in particular acid addition salts, with inorganic or organic acids, wherein the coolants may be present in stereoisomerically pure form or as mixtures of different stereoisomers, preferably wherein the physiological coolant is selected from the group consisting of the compounds B-01, B-02, B-03, B-04, B-05, B-06, B-07, B-11, B-18 and B-19;
or
wherein the physiological coolant of the general formulae (III), (IV), (VII) or (VIII) is selected from the group consisting of the compounds according to Table 2 below:
TABLE 2

| Compound | Structure | IUPAC name |
|---|---|---|
| A-01 | 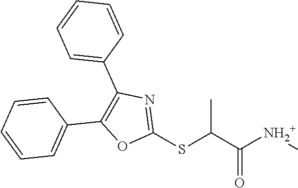 [SMILES: C[NH2+]C(=O)C(C)Sc1nc(-c2ccccc2)c(-c2ccccc2)o1] 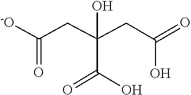 | 3-carboxy-3,5-dihydroxy-5- oxo-pentanoate;2-(4,5- diphenyloxazol-2- yl)sulfanylpropanoyl-methyl- ammonium |
| A-02 | 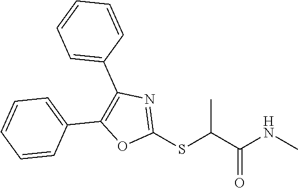 [SMILES: CNC(=O)C(C)Sc1nc(-c2ccccc2)c(-c2ccccc2)o1] | 2-(4,5-diphenyloxazol-2- yl)sulfanyl-N-methyl- propanamide |
| A-03 | 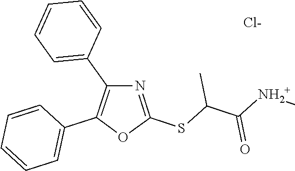 [SMILES: C[NH2+]C(=O)C(C)Sc1nc(-c2ccccc2)c(-c2ccccc2)o1 ; [Cl-]] | 2-(4,5-diphenyloxazol-2- yl)sulfanylpropanoyl-methyl- ammonium;chloride |
| A-04 | 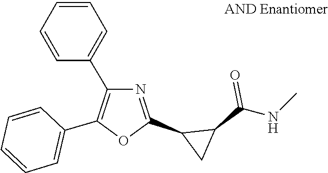 [SMILES: CNC(=O)[C@H]1C[C@H]1c1nc(-c2ccccc2)c(-c2ccccc2)o1] | rac-(1S,2R)-2-(4,5- diphenyloxazol-2-yl)-N- methyl- cyclopropanecarboxamides |
| A-05 | 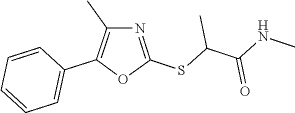 [SMILES: CNC(=O)C(C)Sc1nc(C)c(-c2ccccc2)o1] | N-methyl-2-(4-methyl-5- phenyl-oxazol-2-yl)sulfanyl- propanamide |
| A-06 | 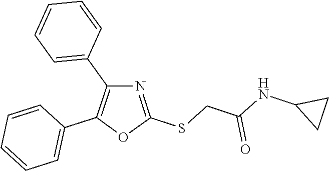 [SMILES: O=C(CSc1nc(-c2ccccc2)c(-c2ccccc2)o1)NC1CC1] | N-cyclopropyl-2-(4,5- diphenyloxazol-2-yl)sulfanyl- acetamide |
| A-07 | 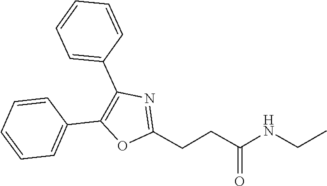 [SMILES: CCNC(=O)CCc1nc(-c2ccccc2)c(-c2ccccc2)o1] | 3-(4,5-diphenyloxazol-2-yl)- N-ethyl-propanamide |
| A-08 | 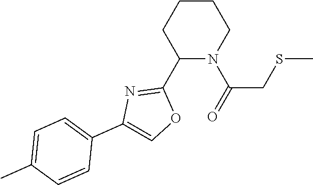 | 2-methylsulfanyl-1-[2-[4-(p- tolyl)oxazol-2-yl]-1- piperidyllethanone |
| A-09 | 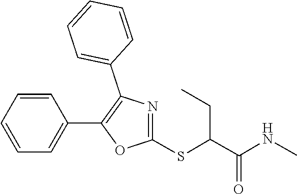 [SMILES: CCC(Sc1nc(-c2ccccc2)c(-c2ccccc2)o1)C(=O)NC] | 2-(4,5-diphenyloxazol-2- yl)sulfanyl-N-methyl- butanamide |
| A-10 | 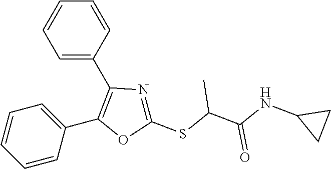 [SMILES: CC(Sc1nc(-c2ccccc2)c(-c2ccccc2)o1)C(=O)NC1CC1] | N-cyclopropyl-2-(4,5- diphenyloxazol-2-yl)sulfanyl- propanamide |
| A-11 | 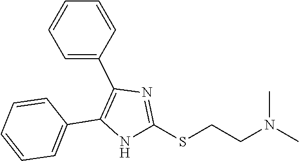 [SMILES: CN(C)CCSc1nc(-c2ccccc2)c(-c2ccccc2)[nH]1] | 2-[(4,5-diphenyl-1H- imidazol-2-yl)sulfanyl]-N,N- dimethyl-ethanamine |
| A-12 | 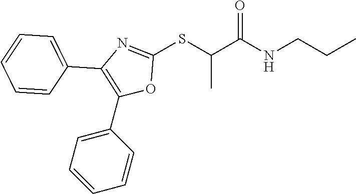 [SMILES: CCCNC(=O)C(C)Sc1nc(-c2ccccc2)c(-c2ccccc2)o1] | 2-(4,5-diphenyloxazol-2- yl)sulfanyl-N-propyl- propanamide |
| A-15 | 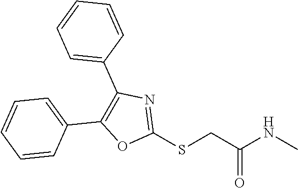 [SMILES: CNC(=O)CSc1nc(-c2ccccc2)c(-c2ccccc2)o1] | 2-(4,5-diphenyloxazol-2- yl)sulfanyl-N-methyl- acetamide |
| A-16 | 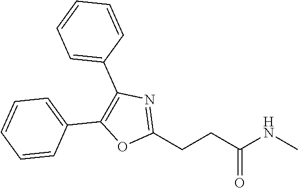 [SMILES: CNC(=O)CCc1nc(-c2ccccc2)c(-c2ccccc2)o1] | 3-(4,5-diphenyloxazol-2-yl)- N-methyl-propanamide |
| A-17 | 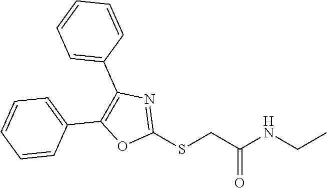 [SMILES: CCNC(=O)CSc1nc(-c2ccccc2)c(-c2ccccc2)o1] | 2-(4,5-diphenyloxazol-2- yl)sulfanyl-N-ethyl- acetamide |
| A-18 | 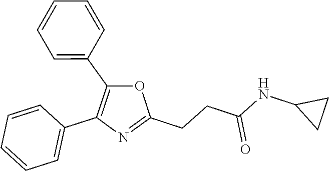 [SMILES: O=C(CCc1nc(-c2ccccc2)c(-c2ccccc2)o1)NC1CC1] | N-cyclopropyl-3-(4,5- diphenyloxazol-2- yl)propanamide |
| A-19 | 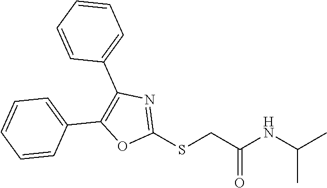 [SMILES: CC(C)NC(=O)CSc1nc(-c2ccccc2)c(-c2ccccc2)o1] | 2-(4,5-diphenyloxazol-2- yl)sulfanyl-N-isopropyl- acetamide |
| A-20 | 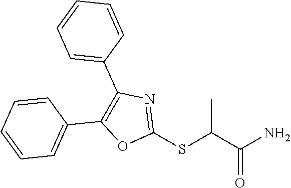 [SMILES: CC(Sc1nc(-c2ccccc2)c(-c2ccccc2)o1)C(N)=O] | 2-(4,5-diphenyloxazol-2- yl)sulfanylpropanamide |
| A-21 | 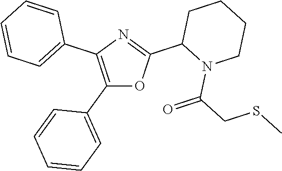 [SMILES: CSCC(=O)N1CCCCC1c1nc(-c2ccccc2)c(-c2ccccc2)o1] | 1-[2-(4,5-diphenyloxazol-2- yl)-1-piperidyl]-2- methylsulfanyl-ethanone |
| A-22 | 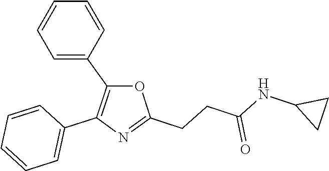 [SMILES: O=C(CCc1nc(-c2ccccc2)c(-c2ccccc2)o1)NC1CC1] | N-cyclopropyl-3-(4,5- diphenyloxazol-2- yl)propanamide |
| A-23 | 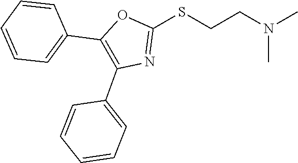 [SMILES: CN(C)CCSc1nc(-c2ccccc2)c(-c2ccccc2)o1] | 2-(4,5-diphenyloxazol-2- yl)sulfanyl-N,N-dimethyl- ethanamine |
| A-24 | 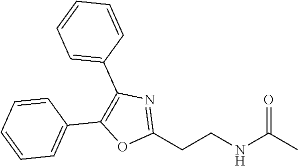 [SMILES: CC(=O)NCCc1nc(-c2ccccc2)c(-c2ccccc2)o1] | N-[2-(4,5-diphenyloxazol-2- yl)ethyl]acetamide |
| A-25 | 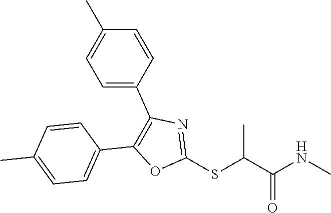 | 2-[4,5-bis(p-tolyl)oxazol-2- yl]sulfanyl-N-methyl- propanamide |
| A-26 | 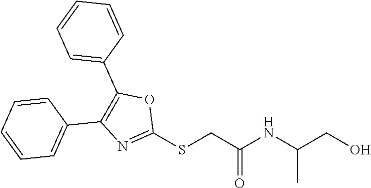 [SMILES: CC(CO)NC(=O)CSc1nc(-c2ccccc2)c(-c2ccccc2)o1] | 2-(4,5-diphenyloxazol-2- yl)sulfanyl-N-(2-hydroxy-1- methyl-ethyl)acetamide |
| A-27 | 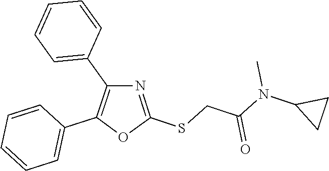 [SMILES: CN(C(=O)CSc1nc(-c2ccccc2)c(-c2ccccc2)o1)C1CC1] | N-cyclopropyl-2-(4,5- diphenyloxazol-2-yl)sulfanyl- N-methyl-acetamide |
| A-28 | 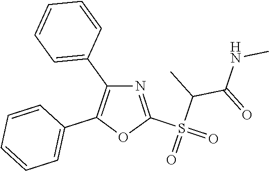 [SMILES: CNC(=O)C(C)S(=O)(=O)c1nc(-c2ccccc2)c(-c2ccccc2)o1] | 2-(4,5-diphenyloxazol-2- yl)sulfonyl-N-methyl- propanamide |
| A-29 | 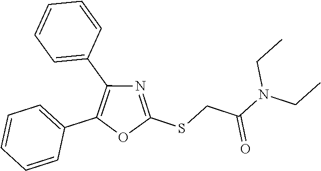 [SMILES: CCN(CC)C(=O)CSc1nc(-c2ccccc2)c(-c2ccccc2)o1] | 2-(4,5-diphenyloxazol-2- yl)sulfanyl-N,N-diethyl- acetamide |
| A-30 | 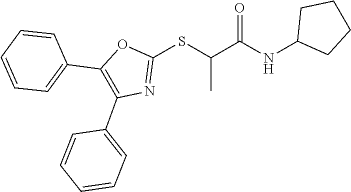 [SMILES: CC(Sc1nc(-c2ccccc2)c(-c2ccccc2)o1)C(=O)NC1CCCC1] | N-cyclopentyl-2-(4,5- diphenyloxazol-2-yl)sulfanyl- propanamide |
| A-31 | 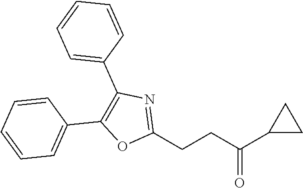 [SMILES: O=C(CCc1nc(-c2ccccc2)c(-c2ccccc2)o1)C1CC1] | 1-cyclopropyl-3-(4,5- diphenyloxazol-2-yl)propan- 1-one |
| A-32 | 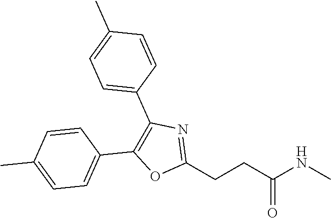 | 3-[4,5-bis(p-tolyl)oxazol-2- yl]-N-methyl-propanamide |
| A-33 | 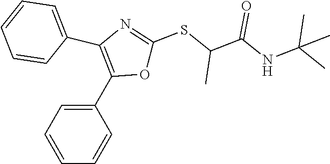 [SMILES: CC(Sc1nc(-c2ccccc2)c(-c2ccccc2)o1)C(=O)NC(C)(C)C] | N-tert-butyl-2-(4,5- diphenyloxazol-2-yl)sulfanyl- propanamide |
| A-34 | 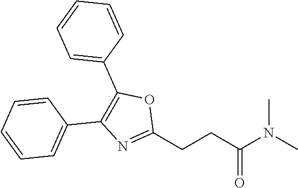 [SMILES: CN(C)C(=O)CCc1nc(-c2ccccc2)c(-c2ccccc2)o1] | 3-(4,5-diphenyloxazol-2-yl)- N,N-dimethyl-propanamide |
| A-35 | 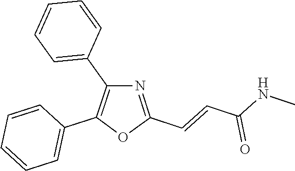 [SMILES: CNC(=O)/C=C/c1nc(-c2ccccc2)c(-c2ccccc2)o1] | (E)-3-(4,5-diphenyloxazol-2- yl)-N-methyl-prop-2-enamide |
| A-36 | 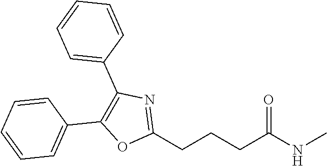 [SMILES: CNC(=O)CCCc1nc(-c2ccccc2)c(-c2ccccc2)o1] | 4-(4,5-diphenyloxazol-2-yl)- N-methyl-butanamide |
| A-37 | 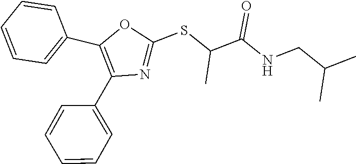 [SMILES: CC(C)CNC(=O)C(C)Sc1nc(-c2ccccc2)c(-c2ccccc2)o1] | 2-(4,5-diphenyloxazol-2- yl)sulfanyl-N-isobutyl- propanamide |
| A-38 | 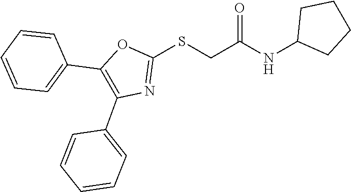 [SMILES: O=C(CSc1nc(-c2ccccc2)c(-c2ccccc2)o1)NC1CCCC1] | N-cyclopentyl-2-(4,5- diphenyloxazol-2-yl)sulfanyl- acetamide |
| A-39 | 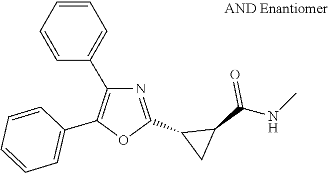 [SMILES: CNC(=O)[C@H]1C[C@@H]1c1nc(-c2ccccc2)c(-c2ccccc2)o1] | rac-(1S,2S)-2-(4,5- diphenyloxazol-2-yl)-N- methyl- cyclopropanecarboxamides |
| A-40 | 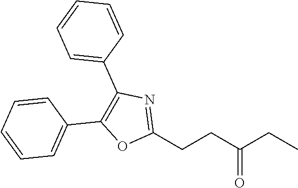 [SMILES: CCC(=O)CCc1nc(-c2ccccc2)c(-c2ccccc2)o1] | 1-(4,5-diphenyloxazol-2- yl)pentan-3-one |
| A-42 | 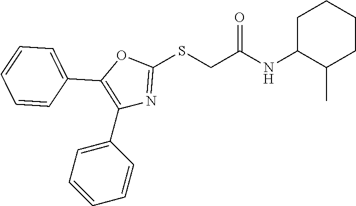 [SMILES: CC1CCCCC1NC(=O)CSc1nc(-c2ccccc2)c(-c2ccccc2)o1] | 2-(4,5-diphenyloxazol-2- yl)sulfanyl-N-(2- methylcyclohexyl)acetamide |
| A-43 | 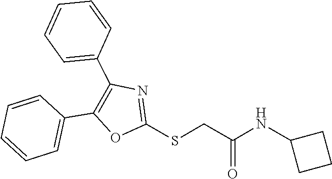 [SMILES: O=C(CSc1nc(-c2ccccc2)c(-c2ccccc2)o1)NC1CCC1] | N-cyclobutyl-2-(4,5- diphenyloxazol-2-yl)sulfanyl- acetamide |
| A-44 | 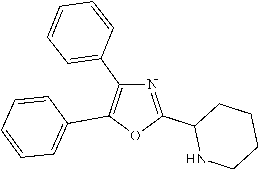 [SMILES: c1ccc(-c2nc(C3CCCCN3)oc2-c2ccccc2)cc1] | 4,5-diphenyl-2-(2- piperidyl)oxazole |
| A-45 | 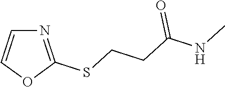 | N-methyl-3-oxazol-2- ylsulfanyl-propanamide |
| A-46 | 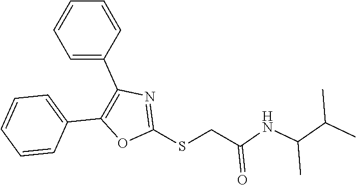 [SMILES: CC(C)C(C)NC(=O)CSc1nc(-c2ccccc2)c(-c2ccccc2)o1] | N-(1,2-dimethylpropyl)-2- (4,5-diphenyloxazol-2- yl)sulfanyl-acetamide |
| A-47 | 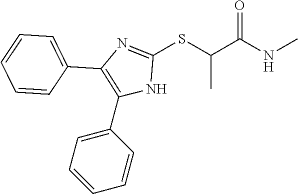 [SMILES: CNC(=O)C(C)Sc1nc(-c2ccccc2)c(-c2ccccc2)[nH]1] | 2-[(4,5-diphenyl-1H- imidazol-2-yl)sulfanyl]-N- methyl-propanamide |
| A-48 | 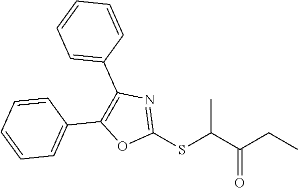 [SMILES: CCC(=O)C(C)Sc1nc(-c2ccccc2)c(-c2ccccc2)o1] | 2-(4,5-diphenyloxazol-2- yl)sulfanylpentan-3-one |
| A-49 | 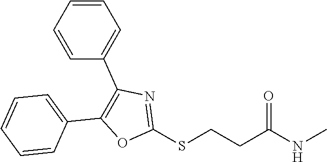 [SMILES: CNC(=O)CCSc1nc(-c2ccccc2)c(-c2ccccc2)o1] | 3-(4,5-diphenyloxazol-2- yl)sulfanyl-N-methyl- propanamide |
| A-50 | 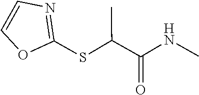 | N-methyl-2-oxazol-2- ylsulfanyl-propanamide |
| A-51 | 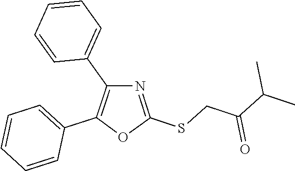 [SMILES: CC(C)C(=O)CSc1nc(-c2ccccc2)c(-c2ccccc2)o1] | 1-(4,5-diphenyloxazol-2- yl)sulfanyl-3-methyl-butan-2- one |
| A-52 | 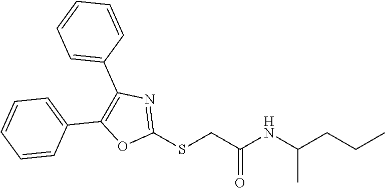 [SMILES: CCCC(C)NC(=O)CSc1nc(-c2ccccc2)c(-c2ccccc2)o1] | 2-(4,5-diphenyloxazol-2- yl)sulfanyl-N-(1- methylbutyl)acetamide |
| A-53 | 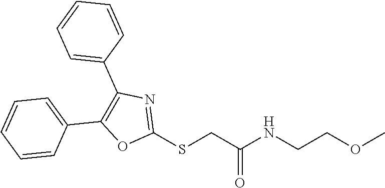 [SMILES: COCCNC(=O)CSc1nc(-c2ccccc2)c(-c2ccccc2)o1] | 2-(4,5-diphenyloxazol-2- yl)sulfanyl-N-(2- methoxyethyl)acetamide |
| A-54 | 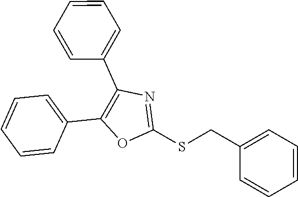 [SMILES: c1ccc(CSc2nc(-c3ccccc3)c(-c3ccccc3)o2)cc1] | 2-benzylsulfanyl-4,5- diphenyl-oxazole |
| A-55 | 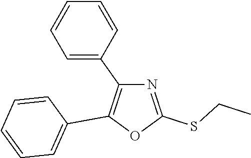 [SMILES: CCSc1nc(-c2ccccc2)c(-c2ccccc2)o1] | 2-ethylsulfanyl-4,5-diphenyl- oxazole |
| A-56 | 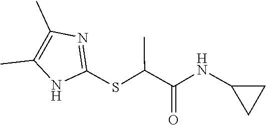 [SMILES: Cc1nc(SC(C)C(=O)NC2CC2)[nH]c1C] | N-cyclopropyl-2-[(4,5- dimethyl-1H-imidazol-2- yl)sulfanyl]propanamide |
| A-57 | 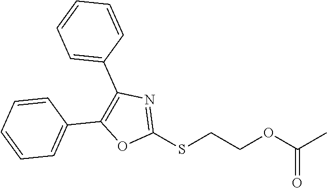 [SMILES: CC(=O)OCCSc1nc(-c2ccccc2)c(-c2ccccc2)o1] | 2-(4,5-diphenyloxazol-2- yl)sulfanylethyl acetate |
| A-58 | 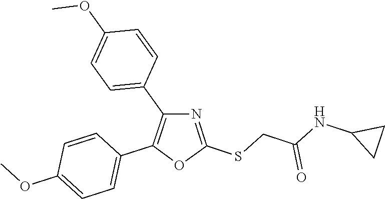 [SMILES: COc1ccc(-c2nc(SCC(=O)NC3CC3)oc2-c2ccc(OC)cc2)cc1] | 2-[4,5-bis(4- methoxyphenyl)oxazol-2- yl]sulfanyl-N-cyclopropyl- acetamide |
| A-59 | 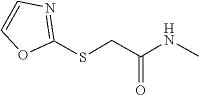 | N-methyl-2-oxazol-2- ylsulfanyl-acetamide |
| A-60 | 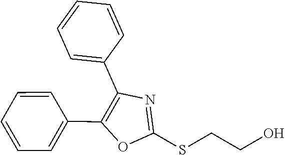 [SMILES: OCCSc1nc(-c2ccccc2)c(-c2ccccc2)o1] | 2-(4,5-diphenyloxazol-2- yl)sulfanylethanol |
| A-61 | 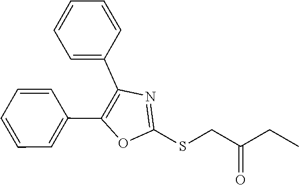 [SMILES: CCC(=O)CSc1nc(-c2ccccc2)c(-c2ccccc2)o1] | 1-(4,5-diphenyloxazol-2- yl)sulfanylbutan-2-one |
| A-62 | 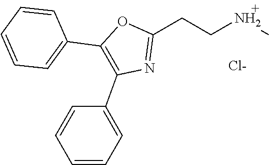 [SMILES: CNCCc1nc(-c2ccccc2)c(-c2ccccc2)o1 ; [Cl-]] | 2-(4,5-diphenyloxazol-2- yl)ethyl-methyl- ammonium;chloride |
| A-63 | 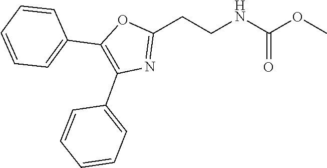 [SMILES: COC(=O)NCCc1nc(-c2ccccc2)c(-c2ccccc2)o1] | methyl N-[2-(4,5- diphenyloxazol-2- yl)ethyl]carbamate |
| A-64 | 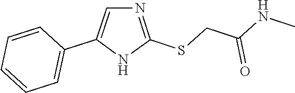 [SMILES: CNC(=O)CSc1ncc(-c2ccccc2)[nH]1] | N-methyl-2-[(5-phenyl-1H- imidazol-2- yl)sulfanyl]acetamide |
| A-65 | 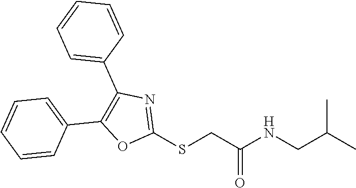 [SMILES: CC(C)CNC(=O)CSc1nc(-c2ccccc2)c(-c2ccccc2)o1] | 2-(4,5-diphenyloxazol-2- yl)sulfanyl-N-isobutyl- acetamide |
| A-66 | 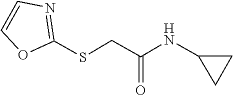 [SMILES: O=C(CSc1ncco1)NC1CC1] | N-cyclopropyl-2-oxazol-2- ylsulfanyl-acetamide |
| A-67 | 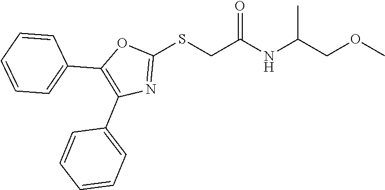 [SMILES: COCC(C)NC(=O)CSc1nc(-c2ccccc2)c(-c2ccccc2)o1] | 2-(4,5-diphenyloxazol-2- yl)sulfanyl-N-(2-methoxy-1- methyl-ethyl)acetamide |
| A-68 | 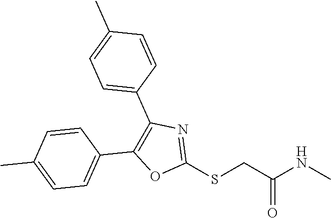 | 2-[4,5-bis(p-tolyl)oxazol-2- yl]sulfanyl-N-methyl- acetamide |
| A-69 | 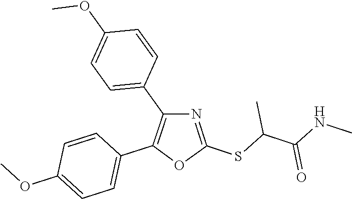 | 2-[4,5-bis(4- methoxyphenyl)oxazol-2- yl]sulfanyl-N-methyl- propanamide |
| A-70 | 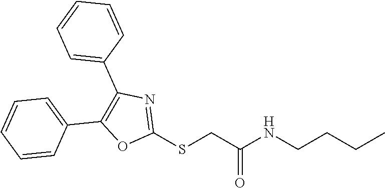 [SMILES: CCCCNC(=O)CSc1nc(-c2ccccc2)c(-c2ccccc2)o1] | N-butyl-2-(4,5- diphenyloxazol-2-yl)sulfanyl- acetamide |
| A-71 | 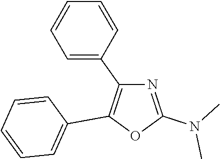 [SMILES: CN(C)c1nc(-c2ccccc2)c(-c2ccccc2)o1] | N,N-dimethyl-4,5-diphenyl- oxazol-2-amine |
| A-72 | 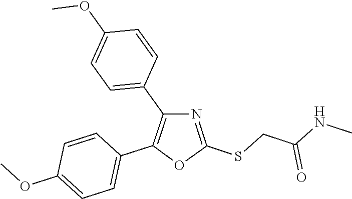 | 2-[4,5-bis(4- methoxyphenyl)oxazol-2- yl]sulfanyl-N-methyl- acetamide |
| A-73 | 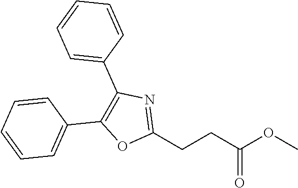 [SMILES: COC(=O)CCc1nc(-c2ccccc2)c(-c2ccccc2)o1] | methyl 3-(4,5- diphenyloxazol-2- yl)propanoate |

and salts thereof, in particular acid addition salts, with inorganic or organic acids, wherein the coolants may be present in stereoisomerically pure form or as mixtures of different stereoisomers, preferably wherein the physiological coolant is selected from the group consisting of the compounds A-02, A-04, A-05, A-06, A-07, A-08, A-09, A-10, A-12, A-16, A-17 and A-18.

12. Physiological coolant according to claim 1, wherein the salt of the coolant is selected from the group consisting of
(1) acid addition salts formed with inorganic acids or formed with organic acids, preferably mono- or polyvalent carboxylic acids; or
(2) salts formed when an acidic proton present in the starting compound is replaced by a metal ion, in particular an alkali metal ion, an alkaline earth ion or an aluminum ion; or coordinated with an organic base.

13. Physiological coolant mixture comprising or consisting of:
(a) one, two, three or more coolant(s) according to claim 1; and optionally
(b) at least one other physiological coolant; and/or optionally
(c) at least one solvent.

14. The physiological coolant mixture according to claim 13, wherein the physiological coolant forming component (b) is selected from the group consisting of: Menthol, Menthol Methyl Ether (FEMA GRAS 4054), Monomenthyl Glutamate (FEMA GRAS 4006), Menthoxy-1,2-propanediol (FEMA GRAS 3784), Dimenthyl Glutarate (FEMA GRAS 4604), hydroxymethylcyclohexylethanone (FEMA GRAS 4742), 2-(4-ethylphenoxy)-N-(1H-pyrazol-3-yl)-N-(thiophen-2-ylmethyl)acetamide (FEMA GRAS 4880), WS-23 (2-isopropyl-N,2,3-trimethylbutyramide, FEMA GRAS 3804), N-(4-(cyanomethyl)phenyl)-2-isopropyl-5,5-dimethylcyclohexane carboxamide (FEMA GRAS 4882), N-(3-hydroxy-4-methoxyphenyl)-2-isopropyl-5,5-dimethylcyclohexane carboxamide (FEMA GRAS 4881), N-(2-hydroxy-2-phenylethyl)-2-isopropyl-5,5-dimethylcyclohexane-1-carboxamide (FEMA GRAS 4896), 3,4-methylenedioxy cinnamic acid, (E)-3-benzo[1,3]dioxol-5-yl-N,N-diphenyl-2-propenamide (FEMA GRAS 4788), menthol propylene glycol carbonate (FEMA GRAS 3806), menthyl N-ethyloxamate, monomethyl succinate (FEMA GRAS 3810), WS-3 (N-ethyl-p-menthane-3-carboxamide, FEMA GRAS 3455), menthol ethylene glycol carbonate (FEMA GRAS 3805), WS-5 (ethyl 3-(p-menthane-3-carboxamido)acetate, FEMA GRAS 4309), WS-12 (1R,2S,5R)—N-(4-methoxyphenyl)-p-menthanecarboxamide (FEMA GRAS 4681), WS-27 (N-ethyl-2,2-diisopropylbutanamide, FEMA GRAS 4557), N-cyclopropyl-5-methyl-2-isopropylcyclohexanecarboxamide (FEMA GRAS 4693), WS-116 (N-(1,1-dimethyl-2-hydroxyethyl)-2,2-diethylbutanamide, FEMA GRAS 4603), menthoxyethanol (FEMA GRAS 4154), N-(4-cyanomethylphenyl)-p-menthanecarboxamide (FEMA GRAS 4496), N-(2-(pyridin-2-yl)ethyl)-3-p-menthanecarboxamide (FEMA GRAS 4549), N-(2-hydroxyethyl)-2-isopropy-1-2,3-dimethylbutanamide (FEMA GRAS 4602), (2S,5R)—N-[4-(2-amino-2-oxoethyl)phenyl]-p-menthanecarboxamide (FEMA GRAS 4684), N-cyclopropyl-5-methyl-2-isopropylcyclohexanecarboxamide (FEMA GRAS 4693), 2-[(2-p-menthoxy)ethoxy]-ethanol (FEMA GRAS 4718), (2,6-diethyl-5-isopropyl-2-methyltetrahydropyran (FEMA GRAS 4680), trans-4-tert-butylcyclohexanol (FEMA GRAS 4724), 2-(p-Tolyloxy)-N-(1H-pyrazol-5-yl)-N-((thiophen-2-yl)methyl)acetamide (FEMA GRAS 4809), menthone glycerol ketal (FEMA GRAS 3807 and 3808), (−)-menthoxypropane-1,2-diol, 3-(1-menthoxy)-2-methylpropane-1,2-diol (FEMA GRAS 3849), isopulegol, (+)-cis and (−)-trans-p-menthane-3,8-diol (62:38, FEMA GRAS 4053), 2,3-dihydroxy-p-menthane, 3,3,5-trimethylcyclohexanone glycerol ketal, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl-3,6,9-trioxadecanoate, (1R,2S,5R)-3-menthyl-3,6,9-trioxadecanoate, (1R,2S,5R)-3-menthyl-(2-hydroxyethoxy)acetate, (1R,2S,5R)-menthyl-11-hydroxy-3,6,9-trioxaundecanoate, cubebol (FEMA GRAS 4497), 2-isopropyl-5-methylcyclohexyl-4-(dimethylamino)-4-oxobutanoate (FEMA GRAS 4230), menthyl lactate (FEMA GRAS 3748), 6-isopropyl-3,9-dimethyl-1,4-dioxaspiro[4.5]decan-2-one (FEMA GRAS 4285), N-benzo[1,3]-dioxol-5-yl-3-p-menthanecarboxamide, N-(1-isopropyl-1,2-dimethylpropyl)-1,3-benzodioxole-5-carboxamide, N—(R)-2-oxotetrahydrofuran-3-yl-(1R,2S,5R)-p-menthane-3-carboxamide, mixture of 2,2,5,6,6-pentamethyl-2,3,6,6a-tetrahydropentalene-3a(1H)-ol and 5-(2-hydroxy-2-methylpropyl)-3,4,4-trimethylcyclopent-2-en-1-one; (2S,5R)-2-isopropyl-5-methyl-N-(2-(pyridin-4-yl)ethyl)cyclohexanecarboxamides; (1S,2S,5R)—N-(4-(cyanomethyl)phenyl)-2-isopropyl-5-methylcyclohexanecarboxamide, 1,7-isopropyl-4,5-methyl-bicyclo[2.2.2]oct-5-ene derivatives, 4-methoxy-N-phenyl-N-[2-(pyridin-2-yl)ethyl]benzamide, 4-methoxy-N-phenyl-N-[2-(pyridin-2-yl)ethyl]benzenesulfonamide, 4-chloro-N-phenyl-N-[2-(pyridin-2-yl)ethyl]benzenesulfonamide, 4-cyano-N-phenyl-N-[2-(pyridin-2-yl)ethyl]-benzenesulfonamide, 4-((benzhydrylamino)methyl)-2-methoxyphenol, 4-((bis(4-methoxyphenyl)methylamino)methyl)-2-methoxyphenol, 4-((1,2-diphenylethylamino)methyl)-2-methoxyphenol, 4-((benzhydryloxy)methyl)-2-methoxyphenol, 4-((9H-fluoren-9-ylamino)methyl)-2-methoxyphenol, 4-((benzhydrylamino)methyl)-2-ethoxyphenol, 1-(4-methoxyphenyl)-2-(1-methyl-1H-benzo[d]imidazol-2-yl)vinyl-4-methoxybenzoate, 2-(1-isopropyl-6-methyl-1H-benzo[d]imidazol-2-yl)-1-(4-methoxyphenyl)vinyl-4-methoxybenzoate, (Z)-2-(1-isopropyl-5-methyl-1H-benzo[d]imidazol-2-yl)-1-(4-methoxyphenyl)vinyl-4-methoxybenzoate, 3-alkyl-p-methan-3-ol derivatives, derivatives of fenchyl, D-bornyl, L-bornyl, exo-norbornyl, 2-methylisobomyl, 2-ethylfenchyl, 2-methylbornyl, cis-pinan-2-yl, verbanyl and isobornyl, menthyloxamate derivatives, menthyl 3-oxocarboxylic acid esters, N-alpha-(menthanecarbonyl)amino acid amides, p-menthane carboxamide and WS-23 analogs, (−)-(1R,2R,4S)-dihydroumbellulol, p-menthane alkyloxyamide, cyclohexane derivatives, butanone derivatives, mixture of 3-menthoxy-1-propanol and 1-menthoxy-2-propanol, 1-[2-ydroxyphenyl]-4-[2-nitrophenyl]-1,2,3,6-tetrahydropyrimidin-2-one, 4-methyl-3-(1-pyrrolidinyl)-2-[5H]-furanone, as well as the coolants according to the following table:

| Structure | Name |
|---|---|
| 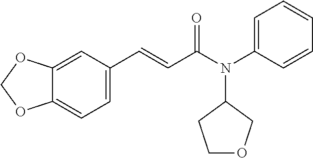 [SMILES: O=C(/C=C/c1ccc2c(c1)OCO2)N(c1ccccc1)C1CCOC1] | (E)-3-(1,3-benzodioxol-5-yl)-N-phenyl-N- tetrahydrofuran-3-yl-prop-2-enamide |
| 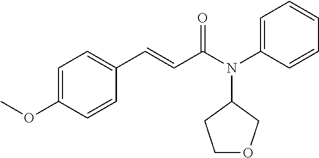 [SMILES: COc1ccc(/C=C/C(=O)N(c2ccccc2)C2CCOC2)cc1] | (E)-3-(4-methoxyphenyl)-N-phenyl-N- tetrahydrofuran-3-yl-prop-2-enamide |
| 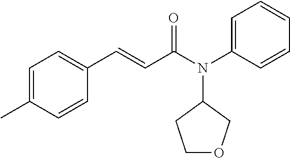 [SMILES: Cc1ccc(/C=C/C(=O)N(c2ccccc2)C2CCOC2)cc1] | (E)-N-phenyl-3-(p-tolyl)-N-tetrahydrofuran-3- yl-prop-2-enamide |
| 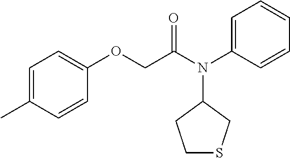 [SMILES: Cc1ccc(OCC(=O)N(c2ccccc2)C2CCSC2)cc1] | 2-(4-Methylphenoxy)-N-phenyl-N- tetrahydrothiophen-3-yl-acetamide |
| 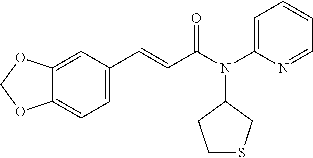 [SMILES: O=C(/C=C/c1ccc2c(c1)OCO2)N(c1ccccn1)C1CCSC1] | (E)-3-(1,3-benzodioxol-5-yl)-N-(2-pyridyl)-N- tetrahydrothiophen-3-yl-prop-2-enamide |
| 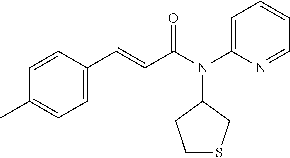 [SMILES: Cc1ccc(/C=C/C(=O)N(c2ccccn2)C2CCSC2)cc1] | (E)-3-(p-tolyl)-N-(2-pyridyl)-N- tetrahydrothiophen-3-yl-prop-2-enamide |
| 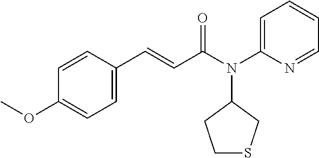 [SMILES: COc1ccc(/C=C/C(=O)N(c2ccccn2)C2CCSC2)cc1] | (E)-3-(4-methoxyphenyl)-N-(2-pyridyl)-N- tetrahydrothiophen-3-yl-prop-2-enamide |
| 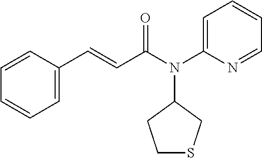 [SMILES: O=C(/C=C/c1ccccc1)N(c1ccccn1)C1CCSC1] | (E)-3-phenyl-N-(2-pyridyl)-N- tetrahydrothiophen-3-yl-prop-2-enamide |
| 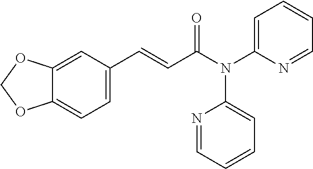 [SMILES: O=C(/C=C/c1ccc2c(c1)OCO2)N(c1ccccn1)c1ccccn1] | (E)-3-(1,3-benzodioxol-5-yl)-N,N-bis(2- pyridyl)prop-2-enamide |
| 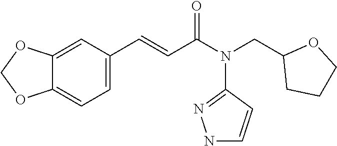 [SMILES: O=C(/C=C/c1ccc2c(c1)OCO2)N(CC1CCCO1)c1cc[nH]n1] | (E)-3-(1,3-benzodioxol-5-yl)-N-(1H-pyrazol- 3-yl)-N-(tetrahydrofuran-2-ylmethyl)prop-2- enamide |
| 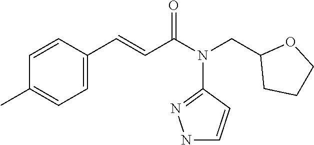 [SMILES: Cc1ccc(/C=C/C(=O)N(CC2CCCO2)c2cc[nH]n2)cc1] | (E)-3-(p-tolyl)-N-(1H-pyrazol-3-yl)-N- (tetrahydrofuran-2-ylmethyl)prop-2-enamide |
| 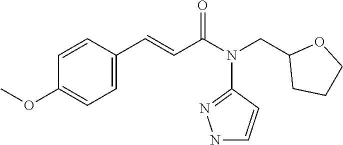 [SMILES: COc1ccc(/C=C/C(=O)N(CC2CCCO2)c2cc[nH]n2)cc1] | (E)-3-(4-methoxyphenyl)-N-(1H-pyrazol-3- yl)-N-(tetrahydrofuran-2-ylmethyl)prop-2- enamide |
| 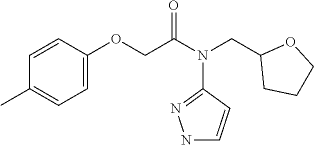 [SMILES: Cc1ccc(OCC(=O)N(CC2CCCO2)c2cc[nH]n2)cc1] | 2-(4-Methylphenoxy)-N-(1H-pyrazol-3-yl)-N- (tetrahydrofuran-2-ylmethyl)acetamide |
| 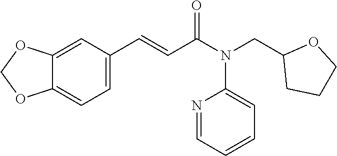 [SMILES: O=C(/C=C/c1ccc2c(c1)OCO2)N(CC1CCCO1)c1ccccn1] | (E)-3-(1,3-benzodioxol-5-yl)-N-(2-pyridyl)-N- (tetrahydrofuran-2-ylmethyl)prop-2-enamide |
| 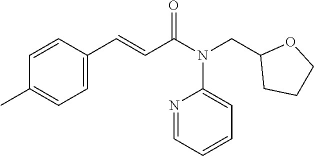 [SMILES: Cc1ccc(/C=C/C(=O)N(CC2CCCO2)c2ccccn2)cc1] | (E)-3-(p-tolyl)-N-(2-pyridyl)-N- (tetrahydrofuran-2-ylmethyl)prop-2-enamide |
| 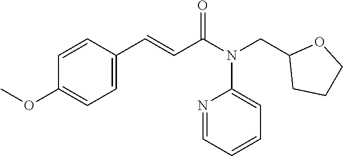 [SMILES: COc1ccc(/C=C/C(=O)N(CC2CCCO2)c2ccccn2)cc1] | (E)-3-(4-methoxyphenyl)-N-(2-pyridyl)-N- (tetrahydrofuran-2-ylmethyl)prop-2-enamide |
| 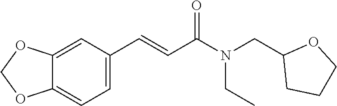 [SMILES: CCN(CC1CCCO1)C(=O)/C=C/c1ccc2c(c1)OCO2] | (E)-3-(1,3-benzodioxol-5-yl)-N-ethyl-N- (tetrahydrofuran-2-ylmethyl)prop-2-enamide |
| 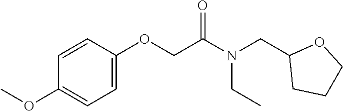 [SMILES: CCN(CC1CCCO1)C(=O)COc1ccc(OC)cc1] | N-ethyl-2-(4-methoxyphenoxy)-N- (tetrahydrofuran-2-ylmethyl)acetamide |
| 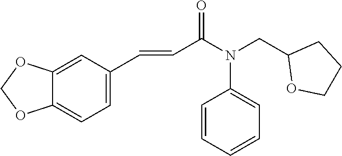 [SMILES: O=C(/C=C/c1ccc2c(c1)OCO2)N(CC1CCCO1)c1ccccc1] | (E)-3-(1,3-benzodioxol-5-yl)-N-phenyl-N- (tetrahydrofuran-2-ylmethyl)prop-2-enamide |
| 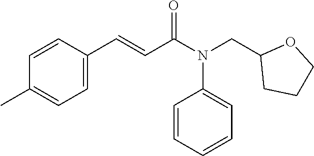 [SMILES: Cc1ccc(/C=C/C(=O)N(CC2CCCO2)c2ccccc2)cc1] | (E)-N-phenyl-3-(p-tolyl)-N-(tetrahydrofuran- 2-ylmethyl)prop-2-enamide |
| 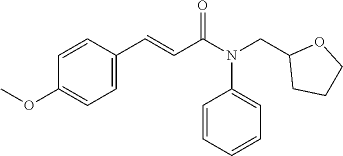 [SMILES: COc1ccc(/C=C/C(=O)N(CC2CCCO2)c2ccccc2)cc1] | (E)-3-(4-methoxyphenyl)-N-phenyl-N- (tetrahydrofuran-2-ylmethyl)prop-2-enamide |
| 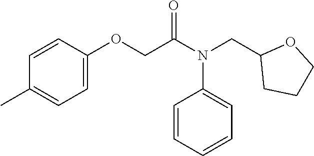 [SMILES: Cc1ccc(OCC(=O)N(CC2CCCO2)c2ccccc2)cc1] | 2-(4-methylphenoxy)-N-phenyl-N- (tetrahydrofuran-2-ylmethyl)acetamide |
| 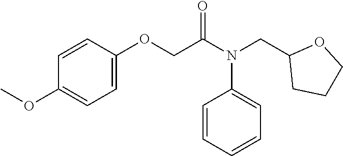 [SMILES: COc1ccc(OCC(=O)N(CC2CCCO2)c2ccccc2)cc1] | 2-(4-methoxyphenoxy)-N-phenyl-N- (tetrahydrofuran-2-ylmethyl)acetamide |
| 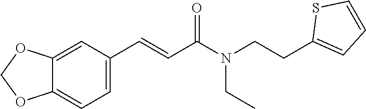 [SMILES: CCN(CCc1cccs1)C(=O)/C=C/c1ccc2c(c1)OCO2] | (E)-3-(1,3-Benzodioxol-5-yl)-N-ethyl-N-[2-(2- thienyl)ethyl]prop-2-enamid |
| 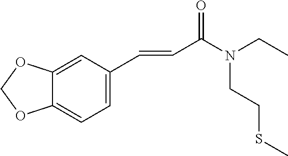 [SMILES: CCN(CCSC)C(=O)/C=C/c1ccc2c(c1)OCO2] | (E)-3-(1,3-benzodioxol-5-yl)-N-ethyl-N-(2- methylsulfanylethyl)prop-2-enamide |
| 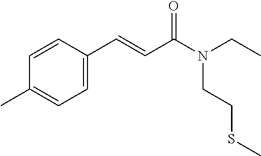 | (E)-N-Ethyl-N-(2-methylsulfanylethyl)-3-(p- tolyl)prop-2-enamide |
| 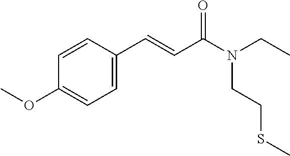 | (E)-N-Ethyl-3-(4-methoxyphenyl)-N-(2- methylsulfanylethyl)prop-2-enamide |
| 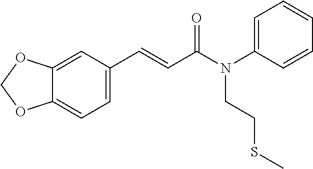 [SMILES: CSCCN(C(=O)/C=C/c1ccc2c(c1)OCO2)c1ccccc1] | (E)-3-(1,3-benzodioxol-5-yl)-N-(2- methylsulfanylethyl)-N-phenyl-prop-2- enamide |
| 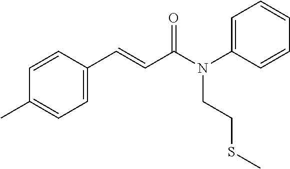 [SMILES: CSCCN(C(=O)/C=C/c1ccc(C)cc1)c1ccccc1] | (E)-N-(2-methylsulfanylethyl)-N-phenyl-3-(p- tolyl)prop-2-enamide |
| 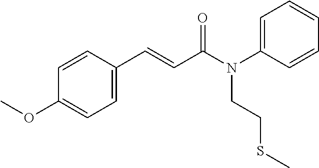 [SMILES: COc1ccc(/C=C/C(=O)N(CCSC)c2ccccc2)cc1] | (E)-3-(4-methoxyphenyl)-N-(2- methylsulfanylethyl)-N-phenyl-prop-2- enamide |
| 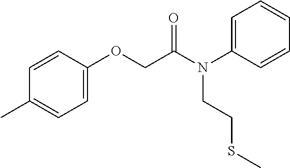 [SMILES: CSCCN(C(=O)COc1ccc(C)cc1)c1ccccc1] | 2-(4-methylphenoxy)-N-(2- methylsulfanylethyl)-N-phenyl-acetamide |
| 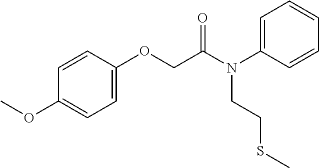 [SMILES: COc1ccc(OCC(=O)N(CCSC)c2ccccc2)cc1] | 2-(4-methoxyphenoxy)-N-(2- methylsulfanylethyl)-N-phenyl-acetamide |
| 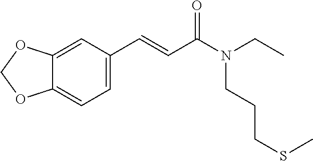 [SMILES: CCN(CCCSC)C(=O)/C=C/c1ccc2c(c1)OCO2] | (E)-3-(1,3-benzodioxol-5-yl)-N-ethyl-N-(3- methylsulfanylpropyl)prop-2-enamide |
| 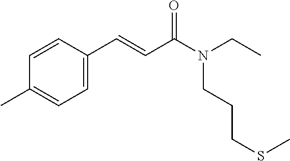 | (E)-N-Ethyl-N-(3-methylsulfanylpropyl)-3-(p- tolyl)prop-2-enamide |
| 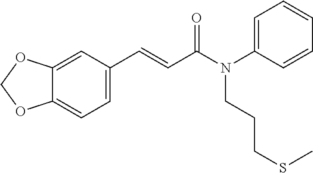 [SMILES: CSCCCN(C(=O)/C=C/c1ccc2c(c1)OCO2)c1ccccc1] | (E)-3-(1,3-benzodioxol-5-yl)-N-(3- methylsulfanylpropyl)-N-phenyl-prop-2- enamide |
| 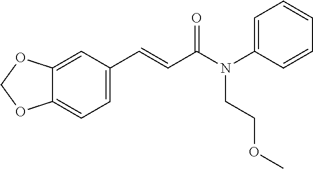 [SMILES: COCCN(C(=O)/C=C/c1ccc2c(c1)OCO2)c1ccccc1] | (E)-3-(1,3-benzodioxol-5-yl)-N-(2- methoxyethyl)-N-phenyl-prop-2-enamide |
| 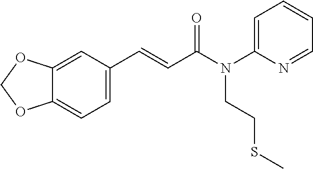 [SMILES: CSCCN(C(=O)/C=C/c1ccc2c(c1)OCO2)c1ccccn1] | (E)-3-(1,3-benzodioxol-5-yl)-N-(2- methylsulfanylethyl)-N-(2-pyridyl)prop-2- enamide |
| 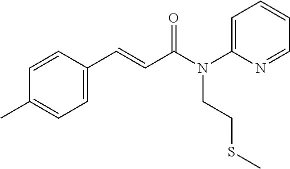 [SMILES: CSCCN(C(=O)/C=C/c1ccc(C)cc1)c1ccccn1] | (E)-N-(2-methylsulfanylethyl)-3-(p-tolyl)-N- (2-pyridyl)prop-2-enamide |
| 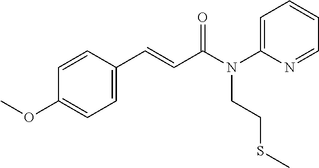 [SMILES: COc1ccc(/C=C/C(=O)N(CCSC)c2ccccn2)cc1] | (E)-3-(4-methoxyphenyl)-N-(2- methylsulfanylethyl)-N-(2-pyridyl)prop-2- enamide |
| 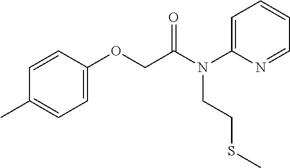 [SMILES: CSCCN(C(=O)COc1ccc(C)cc1)c1ccccn1] | 2-(4-methylphenoxy)-N-(2- methylsulfanylethyl)-N-(2-pyridyl)acetamide |
| 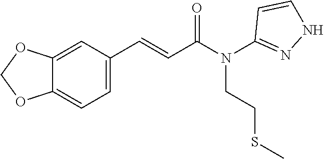 [SMILES: CSCCN(C(=O)/C=C/c1ccc2c(c1)OCO2)c1cc[nH]n1] | (E)-3-(1,3-benzodioxol-5-yl)-N-(2- methylsulfanylethyl)-N-(1H-pyrazol-3- yl)prop-2-enamide |
| 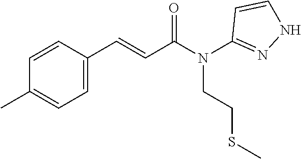 [SMILES: CSCCN(C(=O)/C=C/c1ccc(C)cc1)c1cc[nH]n1] | (E)-N-(2-methylsulfanylethyl)-3-(p-tolyl)-N- (1H-pyrazol-3-yl)prop-2-enamide |
| 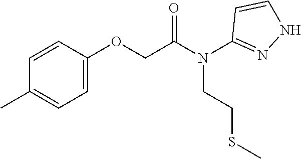 [SMILES: CSCCN(C(=O)COc1ccc(C)cc1)c1cc[nH]n1] | 2-(4-methylphenoxy)-N-(2- methylsulfanylethyl)-N-(1H-pyrazol-3- yl)acetamide |
| 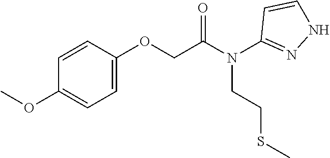 [SMILES: COc1ccc(OCC(=O)N(CCSC)c2cc[nH]n2)cc1] | 2-(4-methoxyphenoxy)-N-(2- methylsulfanylethyl)-N-(1H-pyrazol-3- yl)acetamide |

and the coolants according to the following table:

| Structure | Compound |
|---|---|
| 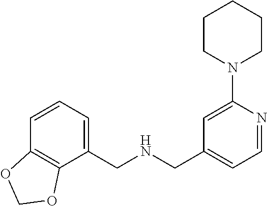 [SMILES: c1cc(CNCc2ccnc(N3CCCCC3)c2)c2c(c1)OCO2] | Compound KS1 |
| 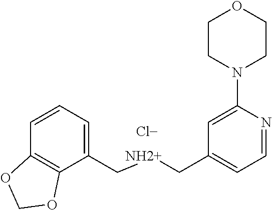 [SMILES: c1cc(C[NH2+]Cc2ccnc(N3CCOCC3)c2)c2c(c1)OCO2 ; [Cl-]] | Compound KS2 |
| 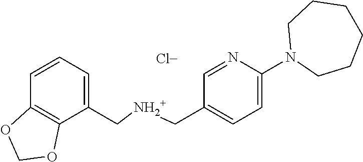 [SMILES: c1cc(C[NH2+]Cc2ccc(N3CCCCCC3)nc2)c2c(c1)OCO2 ; [Cl-]] | Compound KS3 |
| 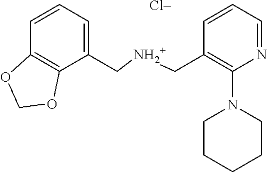 [SMILES: c1cnc(N2CCCCC2)c(C[NH2+]Cc2cccc3c2OCO3)c1 ; [Cl-]] | Compound KS4 |
| 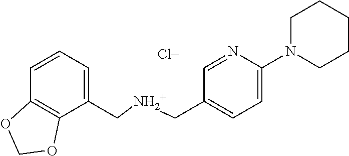 [SMILES: c1cc(C[NH2+]Cc2ccc(N3CCCCC3)nc2)c2c(c1)OCO2 ; [Cl-]] | Compound KS5 |
| 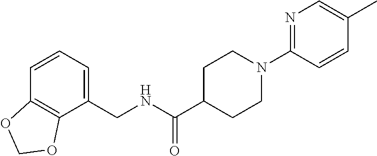 [SMILES: Cc1ccc(N2CCC(C(=O)NCc3cccc4c3OCO4)CC2)nc1] | Compound KS5 a |
| 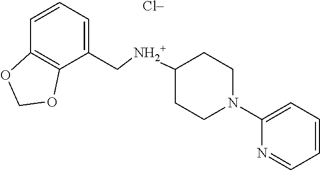 [SMILES: c1ccc(N2CCC([NH2+]Cc3cccc4c3OCO4)CC2)nc1 ; [Cl-]] | Compound KS6 |
| 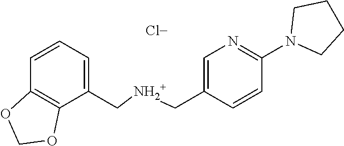 [SMILES: c1cc(C[NH2+]Cc2ccc(N3CCCC3)nc2)c2c(c1)OCO2 ; [Cl-]] | Compound KS7 |
| 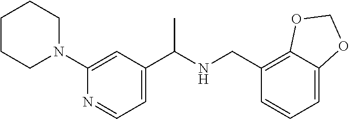 [SMILES: CC(NCc1cccc2c1OCO2)c1ccnc(N2CCCCC2)c1] | Compound KS8 |
| 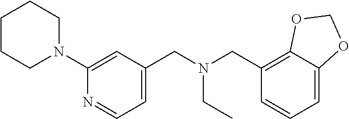 [SMILES: CCN(Cc1ccnc(N2CCCCC2)c1)Cc1cccc2c1OCO2] | Compound KS9 |
| 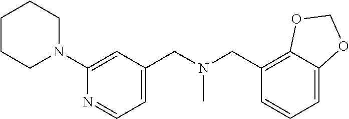 [SMILES: CN(Cc1ccnc(N2CCCCC2)c1)Cc1cccc2c1OCO2] | Compound KS10 |
| 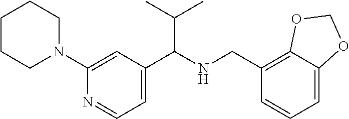 [SMILES: CC(C)C(NCc1cccc2c1OCO2)c1ccnc(N2CCCCC2)c1] | Compound KS11 |
| 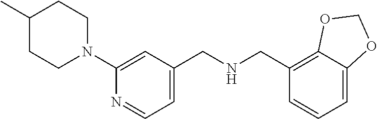 [SMILES: CC1CCN(c2cc(CNCc3cccc4c3OCO4)ccn2)CC1] | Compound KS12 |
| 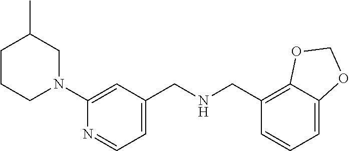 [SMILES: CC1CCCN(c2cc(CNCc3cccc4c3OCO4)ccn2)C1] | Compound KS13 |
| 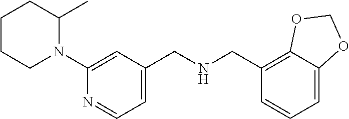 [SMILES: CC1CCCCN1c1cc(CNCc2cccc3c2OCO3)ccn1] | Compound KS14 |
| 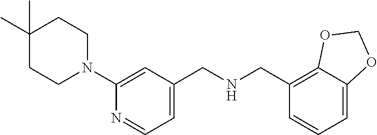 [SMILES: CC1(C)CCN(c2cc(CNCc3cccc4c3OCO4)ccn2)CC1] | Compound KS15 |
| 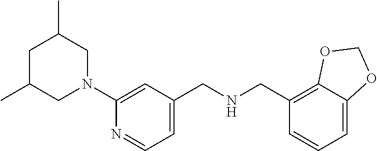 [SMILES: CC1CC(C)CN(c2cc(CNCc3cccc4c3OCO4)ccn2)C1] | Compound KS16 |
| 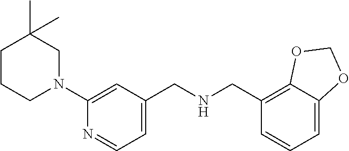 [SMILES: CC1(C)CCCN(c2cc(CNCc3cccc4c3OCO4)ccn2)C1] | Compound KS17 |
| 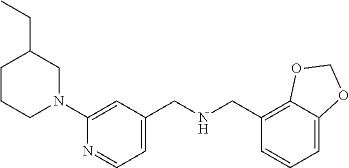 [SMILES: CCC1CCCN(c2cc(CNCc3cccc4c3OCO4)ccn2)C1] | Compound KS18 |
| 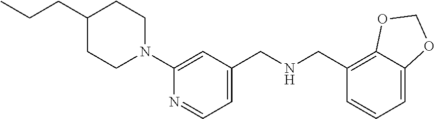 [SMILES: CCCC1CCN(c2cc(CNCc3cccc4c3OCO4)ccn2)CC1] | Compound KS18 a |
| 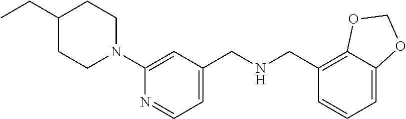 [SMILES: CCC1CCN(c2cc(CNCc3cccc4c3OCO4)ccn2)CC1] | Compound KS19 |
| 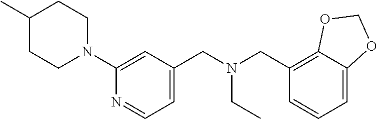 [SMILES: CCN(Cc1ccnc(N2CCC(C)CC2)c1)Cc1cccc2c1OCO2] | Compound KS19 a |
| 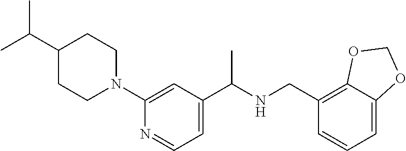 [SMILES: CC(NCc1cccc2c1OCO2)c1ccnc(N2CCC(C(C)C)CC2)c1] | Compound KS20 |
| 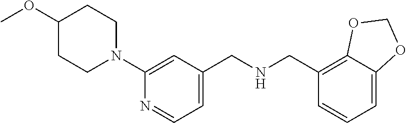 [SMILES: COC1CCN(c2cc(CNCc3cccc4c3OCO4)ccn2)CC1] | Compound KS21 |
| 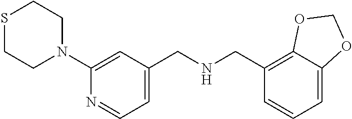 [SMILES: c1cc(CNCc2ccnc(N3CCSCC3)c2)c2c(c1)OCO2] | Compound KS22 |
| 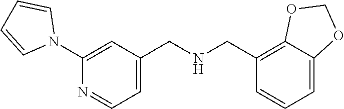 [SMILES: c1cc(CNCc2ccnc(-n3cccc3)c2)c2c(c1)OCO2] | Compound KS22 a |
| 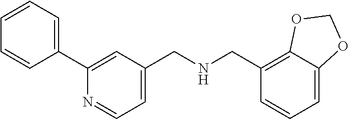 [SMILES: c1ccc(-c2cc(CNCc3cccc4c3OCO4)ccn2)cc1] | Compound KS22 b |
| 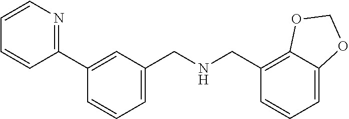 [SMILES: c1ccc(-c2cccc(CNCc3cccc4c3OCO4)c2)nc1] | Compound KS22 c |
| 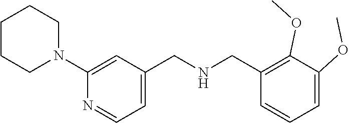 [SMILES: COc1cccc(CNCc2ccnc(N3CCCCC3)c2)c1OC] | Compound KS23 |
| 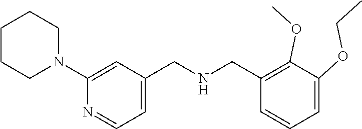 [SMILES: CCOc1cccc(CNCc2ccnc(N3CCCCC3)c2)c1OC] | Compound KS24 |
| 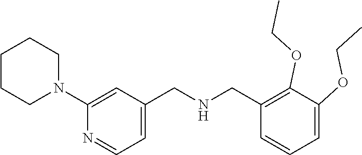 [SMILES: CCOc1cccc(CNCc2ccnc(N3CCCCC3)c2)c1OCC] | Compound KS25 |
| 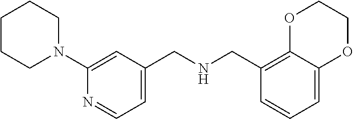 [SMILES: c1cc(CNCc2ccnc(N3CCCCC3)c2)c2c(c1)OCCO2] | Compound KS26 |
| 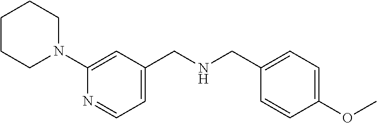 [SMILES: COc1ccc(CNCc2ccnc(N3CCCCC3)c2)cc1] | Compound KS27 |
| 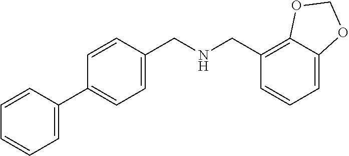 [SMILES: c1ccc(-c2ccc(CNCc3cccc4c3OCO4)cc2)cc1] | Compound KS28 |
| 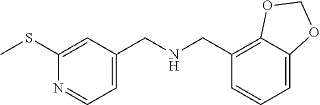 [SMILES: CSc1cc(CNCc2cccc3c2OCO3)ccn1] | Compound KS29 |
| 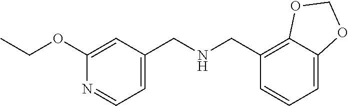 [SMILES: CCOc1cc(CNCc2cccc3c2OCO3)ccn1] | Compound KS30 |
| 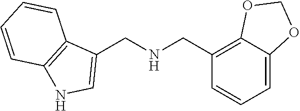 [SMILES: c1cc(CNCc2c[nH]c3ccccc23)c2c(c1)OCO2] | Compound KS31 |
| 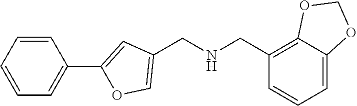 [SMILES: c1ccc(-c2cc(CNCc3cccc4c3OCO4)co2)cc1] | Compound KS31 a |
| 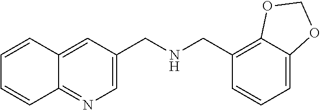 [SMILES: c1cc(CNCc2cnc3ccccc3c2)c2c(c1)OCO2] | Compound KS32 |
| 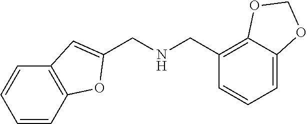 [SMILES: c1cc(CNCc2cc3ccccc3o2)c2c(c1)OCO2] | Compound KS32 a |
| 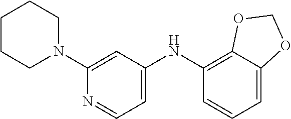 [SMILES: c1cc(Nc2ccnc(N3CCCCC3)c2)c2c(c1)OCO2] | Compound KS33 |
| 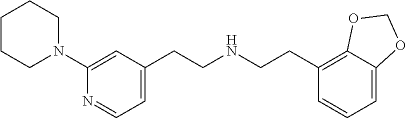 [SMILES: c1cc(CCNCCc2ccnc(N3CCCCC3)c2)c2c(c1)OCO2] | Compound KS34 |
| 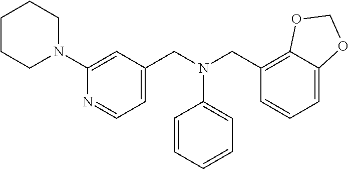 [SMILES: c1ccc(N(Cc2ccnc(N3CCCCC3)c2)Cc2cccc3c2OCO3)cc1] | Compound KS35 |
| 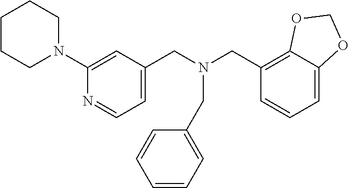 [SMILES: c1ccc(CN(Cc2ccnc(N3CCCCC3)c2)Cc2cccc3c2OCO3)cc1] | Compound KS36 |
| 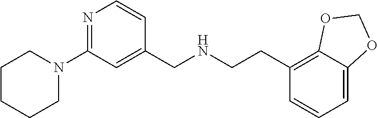 [SMILES: c1cc(CCNCc2ccnc(N3CCCCC3)c2)c2c(c1)OCO2] | Compound KS37 |
| 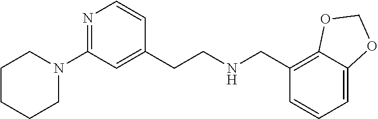 [SMILES: c1cc(CNCCc2ccnc(N3CCCCC3)c2)c2c(c1)OCO2] | Compound KS38 |
| 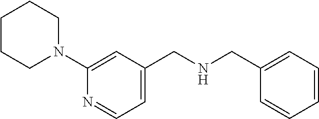 [SMILES: c1ccc(CNCc2ccnc(N3CCCCC3)c2)cc1] | Compound KS39 |
| 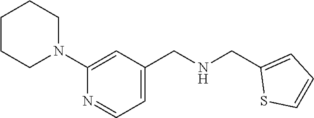 [SMILES: c1csc(CNCc2ccnc(N3CCCCC3)c2)c1] | Compound KS40 |
| 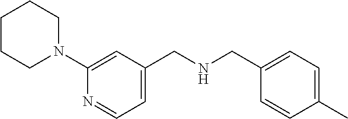 [SMILES: Cc1ccc(CNCc2ccnc(N3CCCCC3)c2)cc1] | Compound KS41 |
| 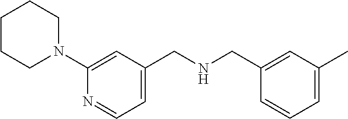 [SMILES: Cc1cccc(CNCc2ccnc(N3CCCCC3)c2)c1] | Compound KS42 |
| 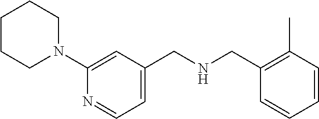 [SMILES: Cc1ccccc1CNCc1ccnc(N2CCCCC2)c1] | Compound KS43 |
| 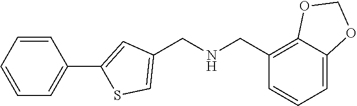 [SMILES: c1ccc(-c2cc(CNCc3cccc4c3OCO4)cs2)cc1] | Compound KS43 a |
| 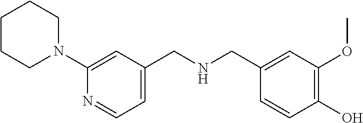 [SMILES: COc1cc(CNCc2ccnc(N3CCCCC3)c2)ccc1O] | Compound KS44 |
| 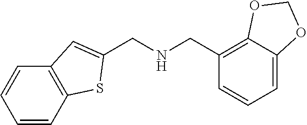 [SMILES: c1cc(CNCc2cc3ccccc3s2)c2c(c1)OCO2] | Compound KS45 |
| 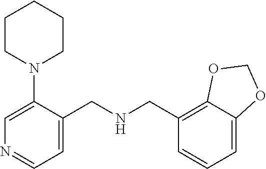 [SMILES: c1cc(CNCc2ccncc2N2CCCCC2)c2c(c1)OCO2] | Compound KS46 |
| 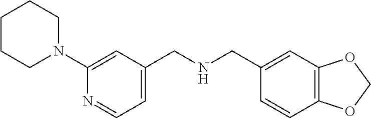 [SMILES: c1cc(CNCc2ccc3c(c2)OCO3)cc(N2CCCCC2)n1] | Compound KS47 |
| 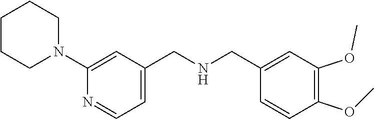 [SMILES: COc1ccc(CNCc2ccnc(N3CCCCC3)c2)cc1OC] | Compound KS48 |
| 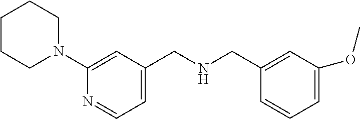 [SMILES: COc1cccc(CNCc2ccnc(N3CCCCC3)c2)c1] | Compound KS49 |
| 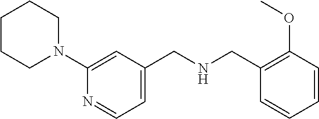 [SMILES: COc1ccccc1CNCc1ccnc(N2CCCCC2)c1] | Compound KS50 |
| 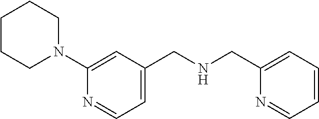 [SMILES: c1ccc(CNCc2ccnc(N3CCCCC3)c2)nc1] | Compound KS51 |
| 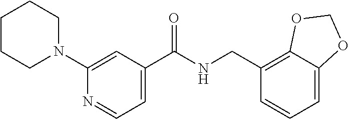 [SMILES: O=C(NCc1cccc2c1OCO2)c1ccnc(N2CCCCC2)c1] | Compound KS51 a |
| 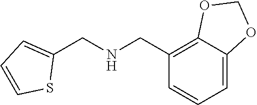 [SMILES: c1csc(CNCc2cccc3c2OCO3)c1] | Compound KS52 |
| 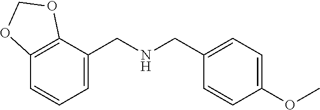 [SMILES: COc1ccc(CNCc2cccc3c2OCO3)cc1] | Compound KS53 |
| 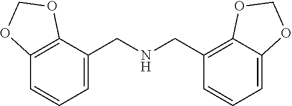 [SMILES: c1cc(CNCc2cccc3c2OCO3)c2c(c1)OCO2] | Compound KS54 |
| 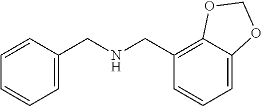 [SMILES: c1ccc(CNCc2cccc3c2OCO3)cc1] | Compound KS55 |
| 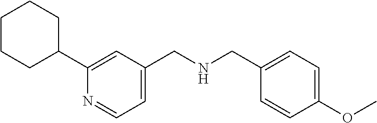 [SMILES: COc1ccc(CNCc2ccnc(C3CCCCC3)c2)cc1] | Compound KS56 |
| 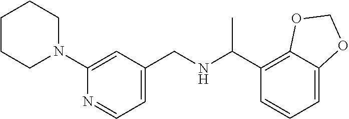 [SMILES: CC(NCc1ccnc(N2CCCCC2)c1)c1cccc2c1OCO2] | Compound KS57 |
| 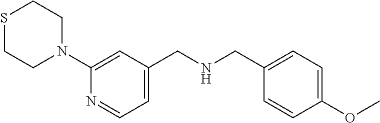 [SMILES: COc1ccc(CNCc2ccnc(N3CCSCC3)c2)cc1] | Compound KS58 |
| 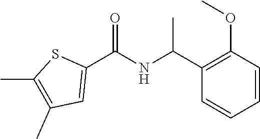 [SMILES: COc1ccccc1C(C)NC(=O)c1cc(C)c(C)s1] | Compound KS59 |
| 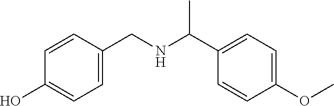 [SMILES: COc1ccc(C(C)NCc2ccc(O)cc2)cc1] | Compound KS60 |
| 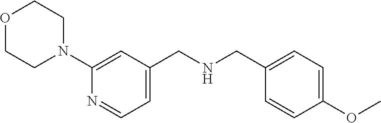 [SMILES: COc1ccc(CNCc2ccnc(N3CCOCC3)c2)cc1] | Compound KS61 |
| 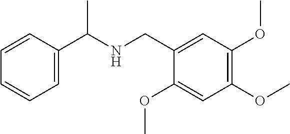 [SMILES: COc1cc(OC)c(OC)cc1CNC(C)c1ccccc1] | Compound KS62 |
| 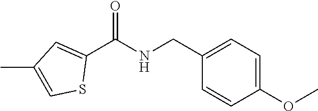 | Compound KS63 |
| 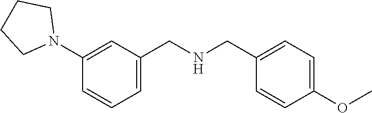 [SMILES: COc1ccc(CNCc2cccc(N3CCCC3)c2)cc1] | Compound KS64 |
| 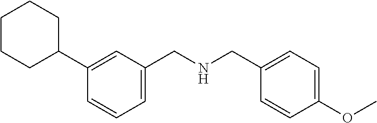 [SMILES: COc1ccc(CNCc2cccc(C3CCCCC3)c2)cc1] | Compound KS65 |
| 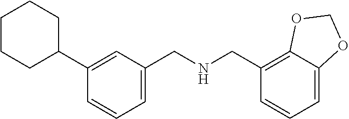 [SMILES: c1cc(CNCc2cccc3c2OCO3)cc(C2CCCCC2)c1] | Compound KS66 |
| 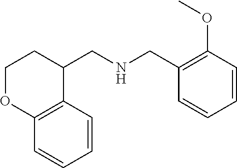 [SMILES: COc1ccccc1CNCC1CCOc2ccccc21] | Compound KS67 |
| 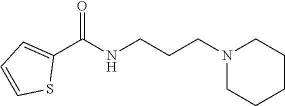 [SMILES: O=C(NCCCN1CCCCC1)c1cccs1] | Compound KS68 |
| 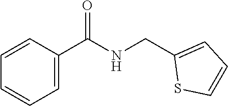 [SMILES: O=C(NCc1cccs1)c1ccccc1] | Compound KS69 |
| 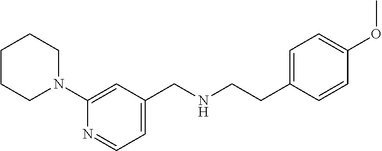 [SMILES: COc1ccc(CCNCc2ccnc(N3CCCCC3)c2)cc1] | Compound KS70 |
| 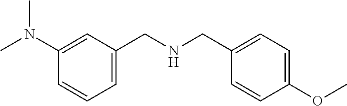 | Compound KS71 |
| 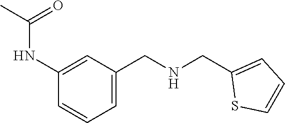 [SMILES: CC(=O)Nc1cccc(CNCc2cccs2)c1] | Compound KS72 |
| 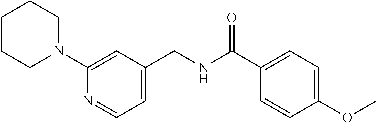 [SMILES: COc1ccc(C(=O)NCc2ccnc(N3CCCCC3)c2)cc1] | Compound KS73 |
| 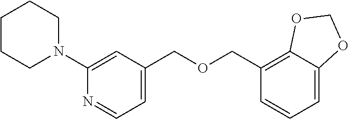 [SMILES: c1cc(COCc2ccnc(N3CCCCC3)c2)c2c(c1)OCO2] | Compound KS74 |
| 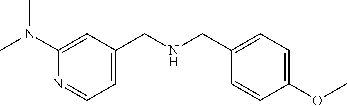 | Compound KS75 |
| 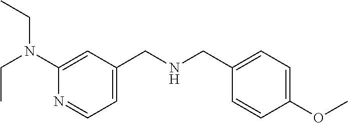 [SMILES: CCN(CC)c1cc(CNCc2ccc(OC)cc2)ccn1] | Compound KS76 |
| 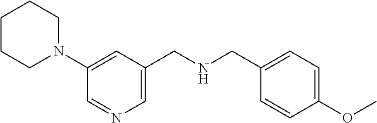 [SMILES: COc1ccc(CNCc2cncc(N3CCCCC3)c2)cc1] | Compound KS77 |
| 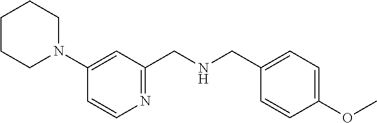 [SMILES: COc1ccc(CNCc2cc(N3CCCCC3)ccn2)cc1] | Compound KS79 |
| 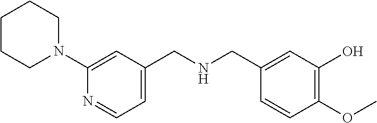 [SMILES: COc1ccc(CNCc2ccnc(N3CCCCC3)c2)cc1O] | Compound KS80 |
| 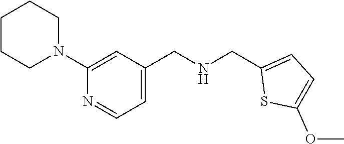 [SMILES: COc1ccc(CNCc2ccnc(N3CCCCC3)c2)s1] | Compound KS81 |
| 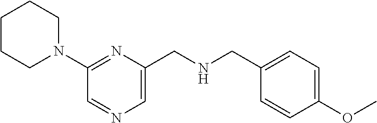 [SMILES: COc1ccc(CNCc2cncc(N3CCCCC3)n2)cc1] | Compound KS82 |
| 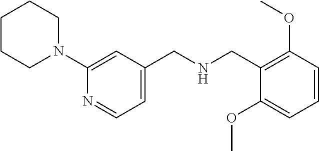 [SMILES: COc1cccc(OC)c1CNCc1ccnc(N2CCCCC2)c1] | Compound KS83 |
| 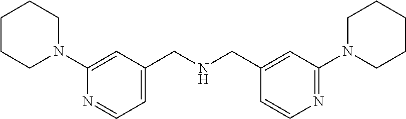 [SMILES: c1cc(CNCc2ccnc(N3CCCCC3)c2)cc(N2CCCCC2)n1] | Compound 84 |
| 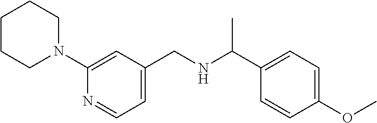 [SMILES: COc1ccc(C(C)NCc2ccnc(N3CCCCC3)c2)cc1] | Compound KS85 |
| 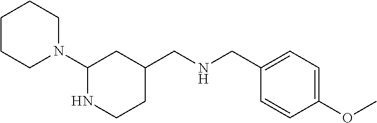 [SMILES: COc1ccc(CNCC2CCNC(N3CCCCC3)C2)cc1] | Compound KS86 |
| 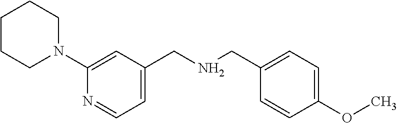 [SMILES: COc1ccc(CNCc2ccnc(N3CCCCC3)c2)cc1] | Compound KS87 |
| 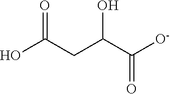 | |
| 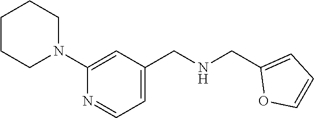 [SMILES: c1coc(CNCc2ccnc(N3CCCCC3)c2)c1] | Compound KS88 |
| 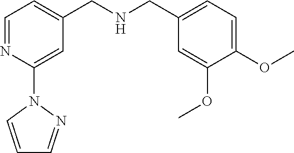 [SMILES: COc1ccc(CNCc2ccnc(-n3cccn3)c2)cc1OC] | Compound KS89 |
| 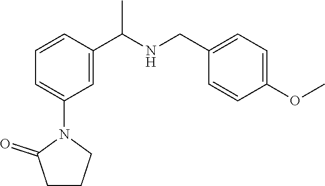 [SMILES: COc1ccc(CNC(C)c2cccc(N3CCCC3=O)c2)cc1] | Compound KS90 |

and their salts, in particular acid addition salts, with inorganic or organic acids and mixtures of the above-mentioned coolants/compounds.

15. Physiological coolant mixture according to claim 13, wherein component (a) and component (b) are present in a weight ratio of from about 0.1:99 to about 99:0.1.

16. The physiological coolant mixture according to claim 13, wherein the solvent forming component (c) is selected from the group consisting of: Benzyl alcohol, 2-phenylethanol, benzyl benzoate, diethyl succinate, triethyl citrate, triacetin, ethanol, peppermint oil, anethole, optamint, propylene glycol, phenoxyethanol and mixtures thereof.

17. Flavoring preparation comprising or consisting of
(d) one, two, three or more coolant(s) according to claim 1 or a physiological coolant mixture comprising one, two, three or more coolant(s) according to claim 1 and optionally at least one other physiological coolant and/or optionally at least one solvent; and
(e) at least one flavoring substance.

18. Flavoring preparation according to claim 17, wherein the flavoring agent forming component (e) is selected from the group consisting of: Acetophenone, allyl capronate, alpha-ionone, beta-ionone, anisaldehyde, anisyl acetate, anisyl formate, anethole, benzaldehyde, benzothiazole, benzyl acetate, benzyl alcohol, benzyl benzoate, beta-ionone, butyl butyrate, butyl capronate, butylidene phthalide, carvone, camphene, caryophyllene, cineole, cinnamyl acetate, citral, citronellol, citronellal, citronellyl acetate, cyclohexyl acetate, cymene, damascone, decalactone, dihydrocoumarin, dimethyl anthranilate, dimethyl anthranilate, dodecalactone, ethoxyethyl acetate, ethyl butyric acid, ethyl butyrate, ethyl caprinate, ethyl capronate, ethyl crotonate, ethyl furaneol, ethyl guaiacol, ethyl isobutyrate, ethyl isovalerate, ethyl lactate, ethyl methyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruit aldehyde, methyldihydrojasmonate (e. g. B. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerate, isobutyl butyrate, isobutyraldehyde, isoeugenol methyl ether, isopropyl methyl thiazole, lauric acid, leavulinic acid, linalool, linalool oxide, linalyl acetate, menthol, menthofuran, methyl anthranilate, methyl butanol, methyl butyric acid, 2-methyl butyl acetate, methyl capronate, methyl cinnamate, 5-methyl furfural, 3,2,2-methyl cyclopentenolone, 6,5,2-methyl heptenone, methyl dihydrojasmonate, methyl jasmonate, 2-methyl methyl butyrate, 2-methyl-2-pentenolic acid, methyl thiobutyrate, 3,1-methyl thiohexanol, 3-methyl thiohexyl acetate, nerol, neryl acetate, trans,trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, piperonal, propionaldehyde, propyl butyrate, pulegone, pulegol, sinensal, sulfurol, terpinene, terpineol, terpinolene, 8,3-thiomenthanone, 4,4,2-thiomethylpentanone, thymol, delta-undecalactone, gamma-undecalactone, valencene, valeric acid, vanillic acid, vanillin, acetoin, ethyl vanillin, ethyl vanillin isobutyrate (=3-ethoxy-4-isobutyryloxybenzaldehyde), 2,5-dimethyl-4-hydroxy-3(2H)-furanone and its derivatives (preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (preferably ethylmaltol), coumarin and coumarin derivatives, gamma-lactones (preferably gamma-undecalactone, gamma-nonalactone, gamma-decalactone), delta-lactones (preferably 4-methyldeltadecalactone, massoilactone, deltadecalactone, tuberolactone), methyl sorbate, divanillin, 4-hydroxy-2(or 5)-ethyl-5(or 2)-methyl-3(2H)-furanone, 2-hydroxy-3-methyl-2-cyclopentenone, 3-hydroxy-4,5-dimethyl-2(5H)-furanone, acetic acid isoamyl ester, butyric acid ethyl ester, butyric acid n-butyl ester, butyric acid isoamyl ester, 3-methyl-butyric acid ethyl ester, n-hexanoic acid ethyl ester, n-hexanoic acid allyl ester, n-hexanoic acid n-butyl ester, n-octanoic acid ethyl ester, ethyl 3-methyl-3-phenylglycidate, ethyl 2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furan, 2-methyl-3-furanethiol, bis(2-methyl-3-furyl)disulfide, furfuryl mercaptan, methional, 2-acetyl-2-thiazoline, 3-mercapto-2-pentanone, 2,5-dimethyl-3-furanethiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guaiacol, 3-hydroxy-4,5-dimethyl-2(5H)-furanone, 3-hydroxy-4-methyl-5-ethyl-2(5H)-furanone, cinnamaldehyde, cinnamyl alcohol, methyl salicylate, isopulegol and stereoisomers, enantiomers, positional isomers, diastereomers, cis/trans isomers or epimers (not explicitly mentioned here) of these substances; and/or epimers of these substances; and/or
wherein the flavoring agent forming component (e) is selected from the group consisting of: Erythritol, Threitol, Arabitol, Ribotol, Xylitol, Sorbitol, Mannitol, Dulcitol, Lactitol, Miraculin, Monellin, Thaumatin, Curculin, Brazzein, Magap, Sodium cyclamate, acesulfame K, neohesperidin dihydrochalcone, saccharin sodium salt, aspartame, superaspartame, neotame, alitame, sucralose, stevioside, rebaudioside, Lugduname, Carrelame, Sucrononate, Sucrooctate, Monatine, Phenylodulcin, Glycine, D-Leucine, D-Threonine, D-Asparagine, D-Phenylalanine, D-tryptophn, L-Proline, Hernandulcin, Dihydrochalconglycosides, Glycyrrhizin, Glycerrhetinic acid, its derivatives and salts, extracts of licorice (Glycyrrhizza glabra ssp.), Lippia dulcis extracts, Momordica ssp. extracts, mogrosides, Hydrangea dulcis and steviosides and mixtures thereof.

19. Flavoring preparation according to claim 17, wherein component (d) and component (e) are present in a weight ratio of from 1:99 to 99:1.

20. Physiological coolant according to claim 1 or a physiological coolant mixture comprising one, two, three or more coolant(s) according to claim 1 and optionally at least one other physiological coolant and/or optionally at least one solvent or a flavoring preparation comprising the physiological coolant or the physiological coolant mixture and at least one flavoring substance in encapsulated form.

21-28. (canceled)
